(19) United States
(12) Patent Application Publication
(10) Pub. No.: US 2023/0085318 A1
(43) Pub. Date: Mar. 16, 2023

(54) POLYNUCLEOTIDES FOR DISRUPTING IMMUNE CELL ACTIVITY AND METHODS OF USE THEREOF

(71) Applicant: ModernaTX, Inc., Cambridge, MA (US)

(72) Inventors: Ying FU, (US); Laurie KENNEY, (US)

(21) Appl. No.: 17/608,340

(22) PCT Filed: May 7, 2020
(86) PCT No.: PCT/US2020/031847

§ 371 Date: Nov. 2, 2021

Related U.S. Application Data
(60) Provisional application No. 62/844,588, filed on May 7, 2019.

(51) Int. Cl. A61K 9/127; C12N 15/62; C12N 5/078; C12N 15/88; C07K 14/705; C07K 14/71; C07K 14/435; A61K 47/28; C12N 9/12; C12N 9/14; C12N 9/16; A61P 37/06

(52) U.S. Cl. CPC A61K 9/127; A61K 38/00; C12N 5/0634; C12N 15/88; C07K 14/705; C07K 14/71; C07K 14/435; C07K 14/70503; C07K 14/70521; C07K 14/70596; A61K 47/28; C12N 9/12; C12Y 207/10002; C12N 9/14; C12Y 306/05002; C12N 9/16; C12Y 301/04011; C12Y 301/03048; C12Y 301/03086; A61P 37/06; C07K 2319/00; C12N 15/625

(57) ABSTRACT

The disclosure features isolated polynucleotides, such as mRNAs, encoding a polypeptide that disrupts immune cell activity, such as T cell or B cell activity, including mRNAs comprising one or more modified nucleobase. The immune cell disruptor polynucleotides encode a polypeptide that comprises a first domain that mediates association of the polypeptide with an immune cell component and a second domain that mediates inhibition of immune cell activity when the polypeptide is expressed in the immune cell. The disclosure also features methods of using the same, for example, for inhibiting immune responses when administered to a subject, such as to inhibit autoimmune reactions.

### CROSS REFERENCE TO RELATED APPLICATIONS

This application claims the benefit of U.S. Provisional Patent Application No. 62/844,588, filed May 7, 2019, the contents of which is incorporated by reference in its entirety.

### BACKGROUND OF THE DISCLOSURE

The ability to downmodulate an immune response is beneficial in a variety of clinical situations, including the treatment of autoimmune diseases, allergies and inflammatory reactions, in prevention of organ transplant rejection and in inhibiting graft-versus-host disease. A number of therapeutic tools exist for downmodulating the function of biological pathways and/or molecules that are involved in aberrant immune responses. These tools include, for example, small molecule inhibitors, cytokines, steroids and therapeutic antibodies. Typically, these tools function through suppressing immune and/or inflammatory responses in a subject, such as small molecule inhibitors (e.g., ciclosporin, azathioprine) that modulate the activity of cells within the immune system, cytokines (e.g., IFN-β) that downmodulate immune responses, or antibodies, such as anti-TNFα and anti-IL2R, that downmodulate immune and/or inflammatory responses. It can be difficult to control the immunosuppressive effects of such agents, however, particularly during long-term, systemic administration. Thus, a common side effect of many immunosuppressive drugs is immunodeficiency, since the majority of these drugs act non-selectively, resulting in increase susceptibility to infections and decreased cancer immunosurveillance. Immune cell depletion can also be an unwanted side effect of certain immunosuppressive agents.

There exists a need in the art for additional effective agents that downmodulate immune responses.

### SUMMARY OF THE DISCLOSURE

This disclosure provides polynucleotides, including messenger RNAs (mRNAs), encoding a polypeptide that inhibits immune cell activity by disrupting normal signaling activity in the cell, referred to herein as immune cell disruptor constructs. In some embodiments, the polypeptide encoded by the polynucleotide (e.g., mRNA) is a chimeric polypeptide that comprises a first portion (i.e., domain or motif) that mediates intracellular association of the polypeptide with an immune cell component. In some embodiments, the immune cell component is a membrane receptor, a membrane-associated protein, a transmembrane associated protein or an intracellular protein, for example intracellular proteins that associate with a membrane protein in the immune cell. In some embodiments, the chimeric polypeptide comprises a second portion (i.e., domain or motif) that mediates inhibition of immune cell activity, such as by disrupting (e.g., altering or inhibiting) normal signaling activity in the immune cell.

In one embodiment, the disclosure provides polynucleotides (e.g., mRNAs) encoding chimeric polypeptides that disrupt, alter or inhibit an activity of a T cell, referred to herein as a T cell disruptor (TCD) construct. In some embodiments, TCD constructs of the disclosure inhibit one or more T cell activities, for example T cell proliferation and/or T cell cytokine production. In other embodiments, the disclosure provides polynucleotides (e.g., mRNAs) encoding chimeric polypeptides that disrupt activity, alter or inhibit an activity of a B cell, referred to herein as a B cell disruptor (BCD) construct. In some embodiments, BCD constructs of the disclosure inhibit one or more B cell activities, for example immunoglobulin production and/or B cell cytokine production. In yet other embodiments, the disclosure provides polynucleotides (e.g., mRNAs) encoding chimeric polypeptides that disrupts, alter or inhibit an activity of an NK cell, for example a dendritic cell or a macrophage. In some embodiments, immune cell activity is inhibited by the immune cell disruptor chimeric polypeptide without substantial or significant depletion of the immune cell.

In one embodiment, the immune cell is a T cell and the disclosure provides polynucleotides (e.g., mRNAs) encoding a T cell disruptor (TCD) construct that inhibits an activity of the T cell. In one embodiment, the polynucleotide (e.g., mRNA) encoding the TCD inhibits T cell proliferation when expressed in the T cell. In one embodiment, the polynucleotide (e.g., mRNA) encoding the TCD inhibits T cell cytokine production when expressed in the T cell.

In one embodiment, the disclosure provides polynucleotides (e.g., mRNAs) encoding a first domain (association domain) of a TCD of a membrane-associated protein expressed in T cells, such as Fyn, Src or KRAS. In some embodiments, the first domain (association domain) of a TCD is an N-terminal membrane-binding portion of human Fyn. In some embodiments, the first domain (association domain) of a TCD is an N-terminal membrane-binding portion of human Src. In some embodiments, the first domain (association domain) of a TCD is or a C-terminal membrane-binding portion of human KRAS.

In other embodiments, the disclosure provides a polynucleotide (e.g., mRNA) encoding a first domain of a transmembrane-associated protein expressed in T cells. In some embodiments, the first domain is PAG, e.g., an N-terminal membrane-binding portion of human PAG. In some embodiments, the disclosure provides a polynucleotide (e.g., mRNA) encoding a first domain of a protein expressed in T cells that associates with a membrane receptor. In some embodiments, the first domain is Lck e.g., a human Lck polypeptide comprising SH2 and SH3 domains. In some embodiments, the first domain is a human ZAP-70 polypeptide comprising at least one SH2 domain. In some embodiments, the disclosure provides polynucleotides (e.g., mRNAs) encoding a first domain of an intracellular protein expressed in T cells, such as LAT, Grb2, Grap, PI3K.p85α, PLCγ1, GADS, ADAP, NCK, VAV, SOS, ITK and SLP76. In some embodiments, the first domain is a human LAT polypeptide selected from a full-length human LAT protein, an N-terminal portion of human LAT and a ZAP-70-binding portion of human LAT. In other embodiments, the first domain is a Grb2 polypeptide comprising an SH2 domain, a Grap polypeptide comprising an SH2 domain, a PI3K.p85α polypeptide in which an internal region containing an iSH2 domain has been deleted or a PLCγ1 polypeptide comprising SH2 and SH3 domains. In one embodiment, the disclosure provides an mRNAs encoding a first domain having an amino acid sequence selected from the group consisting of SEQ ID NOs: 1-20.

In one embodiment, the disclosure provides a polynucleotide (e.g., mRNA) encoding a first domain and at least one second domain of a TCD, wherein the second domain is an inhibitory domain comprising an ITIM motif. In one embodiment, the second domain is a human LAIR1 ITIM1 motif, a human LAIR1 ITIM2 motif or a human CTLA4 ITIM-like motif. In one embodiment, the second domain comprises an inhibitory kinase domain, such as a constitutively active Csk polypeptide, e.g., a constitutively active human Csk polypeptide comprising W47A, R107K and E14A mutations. In one embodiment, the second domain comprises a phosphatase domain, such as a SHP1 polypeptide having phosphatase activity, a SHIP1 polypeptide having phosphatase activity, a PTPN22 polypeptide having phosphatase activity or a PTPN1 polypeptide having phosphatase activity. In one embodiment, the second domain inhibits PI3K activity in the T cell, e.g., the second domain can be from a human PTEN protein. In one embodiment, the disclosure provides an mRNA encoding a second domain having an amino acid sequence selected from the group consisting of SEQ ID NOs: 21-34.

In various embodiments of the polynucleotides (e.g., mRNAs) encoding TCDs of the disclosure, the chimeric polypeptide comprises a first domain from a human LAT protein and a second domain comprising a LAIR1 or CTLA4 ITIM motif. In some embodiments, the polynucleotides (e.g., mRNAs) encoding a TCD of the disclosure comprises a first domain of a human protein selected the group consisting of LAT, PAG, Lck, Fyn and Src and a second domain comprising a constitutively active human CSK protein. In some embodiments, the polynucleotides (e.g., mRNAs) encoding a TCD of the disclosure comprises a first domain from a human protein selected the group consisting of LAT, Src, PI3K.p85 and PLCγ1 and a second domain from a human protein selected from the group consisting of SHP1, SHIP1 and PTPN22. In some embodiments, the polynucleotides (e.g., mRNAs) encoding a TCD of the disclosure comprises a first domain from a human PLCγ1 protein and a second domain from a human PTEN protein.

In one embodiment, an mRNA encoding a TCD of the disclosure comprises a nucleotide sequence shown in any one of SEQ ID NOs: 35-80. In one embodiment, an mRNA encoding TCD of the disclosure encodes a chimeric polypeptide comprising an amino acid sequence shown in any one of SEQ ID NOs: 81-126.

In one embodiment, the immune cell is a B cell and the disclosure provides polynucleotides (e.g., mRNAs) encoding a B cell disruptor (BCD) construct that inhibits an activity of a B cell. In one embodiment, the BCD inhibits B cell immunoglobulin production when expressed in the B cell. In one embodiment, the BCD inhibits B cell cytokine production when expressed in the B cell.

In one embodiment, the disclosure provides polynucleotides (e.g., mRNAs) encoding a BCD construct comprising a first domain of a membrane associated protein expressed in B cells, such as CD79a or CD79b. In one embodiment, the disclosure provides polynucleotides (e.g., mRNAs) encoding a BCD construct comprising a first domain of a human CD79a polypeptide that lacks ITAMs or has inactivated ITAMs or the first domain is a human CD79b polypeptide that lacks ITAMs or has inactivated ITAMs. In one embodiment, the disclosure provides polynucleotides (e.g., mRNAs) encoding a BCD construct comprising a first domain of a membrane receptor expressed in B cells, such as CD19 or CD64. In one embodiment, the disclosure provides polynucleotides (e.g., mRNAs) encoding a BCD construct comprising a first domain of a human CD19 polypeptide that lacks ITAMs or has inactivated ITAMs or the first domain is an N-terminal portion of human CD64. In one embodiment, the disclosure provides polynucleotides (e.g., mRNAs) encoding a BCD construct comprising a first domain of a protein expressed in B cells that associates with a membrane receptor, such as Syk. In one embodiment, the disclosure provides an mRNA encoding a BCD construct comprising a first domain having an amino acid sequence selected from the group consisting of SEQ ID NOs: 127-143 and 229-231.

In one embodiment, the disclosure provides polynucleotides (e.g., mRNAs) encoding a BCD construct comprising a second domain that alters CD19/CD22 balance in the B cell. In one embodiment, the second domain is from CD22 or SHP1, e.g., the second domain comprises a human CD22 ITIM motif or a human SHP1phosphatase domain. In one embodiment, the second domain inhibits B Cell Receptor (BCR) activity in the B cell, e.g., the second domain comprises a CD22 ITIM motif. In one embodiment, the second domain alters FcR activity in the B cell, e.g., the second domain is from CD32b, such as comprising a human CD32b ITIM motif. In one embodiment, the second domain comprises an inhibitory kinase domain, such as a constitutively active Csk polypeptide, e.g., a constitutively active human Csk polypeptide comprising W47A, R107K and E14A mutations. In one embodiment, the disclosure provides an mRNA encoding a BCD construct comprising a second domain having an amino acid sequence selected from the group consisting of SEQ ID NOs: 25, 26 and 144-149.

In various embodiments the disclosure provides polynucleotides (e.g., mRNAs) encoding a BCD construct comprising a chimeric polypeptide comprising a first domain of a human protein selected from the group consisting of CD79a, CD79b, CD19 and Syk and a second domain of a human CD22, human SHP1 or human Csk. In some embodiments, the disclosure provides polynucleotides (e.g., mRNAs) encoding a BCD construct comprising a chimeric polypeptide comprising a first domain from human CD64 and a second domain from human CD32b.

In one embodiment, the disclosure provides an mRNA encoding a BCD of the disclosure comprising a nucleotide sequence shown in any one of SEQ ID NOs: 150-167 and 232-237. In one embodiment, the disclosure provides an mRNA encoding a BCD comprising a chimeric polypeptide comprising an amino acid sequence shown in any one of SEQ ID NOs: 168-185 and 238-243.

In some embodiments, the polynucleotide is a messenger RNA (mRNA). In some embodiments, the mRNA is chemically modified, referred to herein as a modified mRNA, wherein the mRNA comprises one or more modified nucleobases. Alternatively, the mRNA can entirely comprise unmodified nucleobases. In one embodiment, an mRNA or modified mRNA construct of the disclosure comprises, for example, a 5′ UTR, a codon optimized open reading frame encoding the polypeptide, a 3′ UTR and a 3′ tailing region of linked nucleosides. In one embodiment, the mRNA further comprises one or more microRNA (miRNA) binding sites.

In one embodiment, a modified mRNA construct of the disclosure is fully modified. For example, in one embodiment, the mRNA comprises pseudouridine (ψ), pseudouridine (ψ) and 5-methyl-cytidine (m⁵C), 1-methyl-pseudouridine (m¹ψ), 1-methyl-pseudouridine (m¹ψ) and 5-methyl-cytidine (m⁵C), 2-thiouridine (s²U), 2-thiouridine and 5-methyl-cytidine (m⁵C), 5-methoxy-uridine (mo⁵U), 5-methoxy-uridine (mo⁵U) and 5-methyl-cytidine (m⁵C), 2′-O-methyl uridine, 2′-O-methyl uridine and 5-methyl-cytidine (m⁵C), N6-methyl-adenosine (m⁶A) or N6-methyl-adenosine (m⁶A) and 5-methyl-cytidine (m⁵C). In another embodiment, the mRNA comprises pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine, or combinations thereof. In yet another embodiment, the mRNA comprises 1-methyl-pseudouridine (m¹ψ), 5-methoxy-uridine (mo⁵U), 5-methyl-cytidine (m⁵C), pseudouridine (ψ), α-thio-guanosine, or α-thio-adenosine, or combinations thereof.

In another aspect, the disclosure pertains to a lipid nanoparticle comprising a polynucleotide, such as an mRNA (e.g., modified mRNA), of the disclosure. In one embodiment, the lipid nanoparticle is a liposome. In another embodiment, the lipid nanoparticle comprises a cationic and/or ionizable lipid. In one embodiment, the lipid nanoparticle comprises an immune cell delivery potentiating lipid, which promotes delivery of the mRNA into immune cells. In one embodiment, the LNP comprises a phytosterol or a combination of a phytosterol and cholesterol. In one embodiment, the phytosterol is selected from the group consisting of (3-sitosterol, stigmasterol, β-sitostanol, campesterol, brassicasterol, and combinations thereof. In one embodiment, the phytosterol is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

In one embodiment, a lipid nanoparticle is coformulated with two or more mRNA constructs of the disclosure. For example an LNP can be coformulated with at least one T cell disruptor construct (TCD) and at least one B cell disruptor construct (BCD). In one embodiment, the LNP is coformulated with one TCD and three BCDs.

In another aspect, the disclosure pertains to a pharmaceutical composition comprising an mRNA (e.g., modified mRNA) of the disclosure or a lipid nanoparticle of the disclosure, and a pharmaceutically acceptable carrier, diluent or excipient.

In any of the foregoing or related aspects, the disclosure provides a kit comprising a container comprising a lipid nanoparticle, and an optional pharmaceutically acceptable carrier, or a pharmaceutical composition, and a package insert comprising instructions for administration of the lipid nanoparticle or pharmaceutical composition for inhibiting an immune response in an individual. In some aspects, the package insert further comprises instructions for administration of the lipid nanoparticle or pharmaceutical composition alone, or in combination with a composition comprising another immunomodulatory agent, and an optional pharmaceutically acceptable carrier for inhibiting an immune response in an individual.

In any of the foregoing or related aspects, the disclosure provides use of a lipid nanoparticle of the disclosure, and an optional pharmaceutically acceptable carrier, in the manufacture of a medicament for inhibiting an immune response in an individual, wherein the medicament comprises the lipid nanoparticle and an optional pharmaceutically acceptable carrier and wherein the treatment comprises administration of the medicament, and an optional pharmaceutically acceptable carrier.

In another aspect, the disclosure pertains to a method for inhibiting an immune response in a subject, the method comprising administering to a subject in need thereof a polynucleotide composition of disclosure (e.g., mRNA or modified RNA) that inhibits activity of an immune cell, or lipid nanoparticle thereof, or pharmaceutical composition thereof, such that an immune response is inhibited in the subject. In one aspect, inhibiting an immune response in a subject comprises inhibiting cytokine production. In another aspect, inhibiting an immune response in a subject comprises inhibiting immune cell (e.g., T cell or B cell) proliferation. In another aspect, inhibiting an immune response in a subject comprises inhibiting immunoglobulin production (e.g., antigen-specific antibody production).

In any of the foregoing or related aspects, the disclosure provides a method for treating a subject, for example a subject having a disease or condition that would benefit from inhibiting an immune response in the subject. The treatment method comprises administering to a subject in need thereof any of the foregoing or related immunoinhibitory therapeutic compositions or any of the foregoing or related lipid nanoparticle carriers. In some aspects, the immunomodulatory therapeutic composition or lipid nanoparticle carrier is administered in combination with another therapeutic agent (e.g., an autoimmune therapeutic agent, immunosuppressive agent or the like).

In one embodiment, the subject has an autoimmune disease, such as rheumatoid arthritis, systemic lupus erythematosus, inflammatory bowel disease (including ulcerative colitis and Crohn's disease), Type 1 diabetes, multiple sclerosis, psoriasis, Graves' disease, Hashimoto's thyroiditis, chronic inflammatory demyelinating polyneuropathy, Guillain-Barre syndrome, myasthenia gravis, glomerulonephritis or vasculitis. In one embodiment, the subject has an allergic disorder. In one embodiment, the subject has an inflammatory reaction. In one embodiment, the subject is a transplant recipient (e.g., the recipient of a solid organ transplant or a bone marrow transplant, including a subject suffering from GVHD). In one embodiment, the subject is undergoing immunotherapy (e.g., adoptive T cell therapy) and the method is used to downmodulate the immune response that is being stimulated in the subject by the immunotherapy.

In other embodiments, the disclosure provides an immune cell delivery LNP comprising:

(i) an ionizable lipid;

(ii) a sterol or other structural lipid;

(iii) a polynucleotide of the disclosure;

(iv) optionally, a non-cationic helper lipid or phospholipid; and

(v) optionally, a PEG-lipid;

wherein one or more of (i) the ionizable lipid or (ii) the sterol or other structural lipid comprises an immune cell delivery potentiating lipid in an amount effective to enhance delivery of the LNP to a target immune cell, wherein the target immune cell is a T cell or a B cell.

In some aspects, the immune cell delivery LNP comprises a phytosterol or a combination of a phytosterol and cholesterol.

In some aspects, the immune cell delivery LNP comprises a phytosterol, wherein the phytosterol is selected from the group consisting of β-sitosterol, stigmasterol, β-sitostanol, campesterol, brassicasterol, and combinations thereof.

In some aspects, the immune cell delivery LNP comprises a phytosterol, wherein the phytosterol comprises a sitosterol or a salt or an ester thereof.

In some aspects, the immune cell delivery LNP comprises a phytosterol, wherein the phytosterol comprises a stigmasterol or a salt or an ester thereof.

In some aspects, the immune cell delivery LNP comprises a phytosterol, wherein the phytosterol is beta-sitosterol

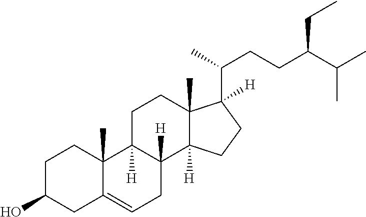

or a salt or an ester thereof.

In some aspects, the immune cell delivery LNP comprises a phytosterol, wherein the phytosterol or a salt or ester thereof is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

In some aspects, the immune cell delivery LNP comprises a phytosterol, wherein the phytosterol is β-sitosterol.

In some aspects, the immune cell delivery LNP comprises a phytosterol, wherein the phytosterol is β-sitostanol.

In some aspects, the immune cell delivery LNP comprises a phytosterol, wherein the phytosterol is campesterol.

In some aspects, the immune cell delivery LNP comprises a phytosterol, wherein the phytosterol is brassicasterol.

In some aspects, the immune cell delivery LNP comprises an ionizable lipid, wherein the ionizable lipid comprises a compound of any of Formulae (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8).

In some aspects, the immune cell delivery LNP comprises an ionizable lipid, wherein the ionizable lipid comprises a compound selected from the group consisting of Compound X, Compound Y, Compound I-48, Compound I-50, Compound I-109, Compound I-111, Compound I-113, Compound I-181, Compound I-182, Compound I-244, Compound I-292, Compound I-301, Compound I-309, Compound I-317, Compound I-321, Compound I-322, Compound I-326, Compound I-328, Compound I-330, Compound I-331, Compound I-332, Compound I-347, Compound I-348, Compound I-349, Compound I-350, Compound I-352 and Compound I-M.

In some aspects, the immune cell delivery LNP comprises an ionizable lipid, wherein the ionizable lipid comprises a compound selected from the group consisting of Compound X, Compound Y, Compound I-321, Compound I-292, Compound I-326, Compound I-182, Compound I-301, Compound I-48, Compound I-50, Compound I-328, Compound I-330, Compound I-109, Compound I-111 and Compound I-181.

In some aspects, the immune cell delivery LNP comprises a phospholipid, wherein the phospholipid comprises a compound selected from the group consisting of DSPC, DMPE, and Compound H-409.

In some aspects, the immune cell delivery LNP comprises a PEG-lipid.

In some aspects, the immune cell delivery LNP comprises a PEG-lipid, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some aspects, the immune cell delivery LNP comprises a PEG lipid, wherein the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25.

In some aspects, the immune cell delivery LNP comprises a PED lipid, wherein the PEG lipid comprises a compound selected from the group consisting of Compound P-428, Compound PL-16, Compound PL-17, Compound PL-18, Compound PL-19, Compound PL-1, and Compound PL-2.

In some aspects, the immune cell delivery LNP comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid.

In some aspects, the immune cell delivery LNP comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid.

In some aspects, the immune cell delivery LNP comprises about 50 mol % ionizable lipid, about 10 mol % non-cationic helper lipid or phospholipid, about 38.5 mol % sterol or other structural lipid, and about 1.5 mol % PEG lipid.

In some aspects, the immune cell delivery LNP comprises 18.5% phytosterol and the total mol % structural lipid is 38.5%.

In some aspects, the immune cell delivery LNP comprises 28.5% phytosterol and the total mol % structural lipid is 38.5%.

In some aspects, the immune cell delivery LNP comprises:

(i) about 50 mol % ionizable lipid, wherein the ionizable lipid is a compound selected from the group consisting of Compound I-301, Compound I-321, and Compound I-326;

(ii) about 10 mol % phospholipid, wherein the phospholipid is DSPC;

(iii) about 38.5 mol % structural lipid, wherein the structural lipid is selected from β-sitosterol and cholesterol; and

(iv) about 1.5 mol % PEG lipid, wherein the PEG lipid is Compound P-428.

In any of the foregoing or related aspects, the disclosure provides use of the immune cell delivery LNP of the disclosure, and an optional pharmaceutically acceptable carrier, in the manufacture of a medicament for inhibiting an immune response in an individual, wherein the medicament comprises the LNP and an optional pharmaceutically acceptable carrier and wherein the treatment comprises administration of the medicament, and an optional pharmaceutically acceptable carrier.

In another aspect, the disclosure pertains to a method for inhibiting an immune response in a subject, the method comprising administering to a subject in need thereof an immune cell delivery LNP of the disclosure, or pharmaceutical composition thereof, such that an immune response is inhibited in the subject. In one aspect, inhibiting an immune response in a subject comprises inhibiting cytokine production. In another aspect, inhibiting an immune response in a subject comprises inhibiting immune cell (e.g., T cell or B cell) proliferation. In another aspect, inhibiting an immune response in a subject comprises inhibiting immunoglobulin production (e.g., antigen-specific antibody production).

In any of the foregoing or related aspects, the disclosure provides a method for treating a subject, for example a subject having a disease or condition that would benefit from inhibiting an immune response in the subject. The treatment method comprises administering to a subject in need thereof any of the foregoing or related immune cell delivery LNPs. In some aspects, the immune cell delivery LNP is administered in combination with another therapeutic agent (e.g., an autoimmune therapeutic agent, immunosuppressive agent or the like).

### BRIEF DESCRIPTION OF THE DRAWINGS

FIGS. 1A-1F are graphs showing inhibition of T cell proliferation by mRNA constructs encoding T cell disruptors (TCDs). FIG. 1A-1C show results for CD4+ T cells treated with either 0.3 μl (FIG. 1A), 1.0 μl (FIG. 1B) or 3.0 μl (FIG. 1C) of T cell activation beads and the TCD constructs shown on the X axis. FIG. 1D-1F show results for CD8+ T cells treated with either 0.3 μl (FIG. 1D), 1.0 μl (FIG. 1E) or 3.0 μl (FIG. 1F) of T cell activation beads and the TCD constructs shown on the X axis. The upper dotted line in each graph represents the level of proliferation observed for cells treated with a negative control mRNA construct (set as 100% proliferation) and the lower dotted line in each graph represents 50% of that (i.e., 50% inhibition of proliferation).

FIGS. 2A-2D are graphs showing inhibition of proliferation of pre-activated T cells by mRNA constructs encoding T cell disruptors (TCDs). FIG. 2A-2B show results for CD4+ T cells treated with the indicated TCD constructs at either 0 hours (FIG. 2A) or 24 hours (FIG. 2B) post T cell activation. FIG. 2C-2D show results for CD8+ T cells treated the indicated TCD constructs at either 0 hours (FIG. 2C) or 24 hours (FIG. 2D) post T cell activation. The upper dotted line in each graph represents the level of proliferation observed for cells treated with a negative control mRNA construct (set as 100% proliferation) and the lower dotted line in each graph represents 50% of that (i.e., 50% inhibition of proliferation).

FIGS. 3A-3B are graphs showing inhibition of TNFα production in T cells by mRNA constructs encoding T cell disruptors (TCDs). FIG. 3A show results for CD4+ T cells treated with the indicated TCD constructs. FIG. 3B show results for CD8+ T cells treated with the indicated TCD constructs. The upper dotted line in each graph represents the level of TNFα production in T cells treated with a negative control mRNA construct (set as 100% production). The middle and lower dotted lines in FIG. 3A represent 50% and 25%, respectively, of that (i.e., 50% or 75% inhibition of TNFα production). The lower dotted line in FIG. 3B represents 50% of maximum (i.e., 50% inhibition of TNFα production).

FIG. 4 is a graph showing that T cell disruptor mRNA constructs delay mortality in a xeno-GVHD animal model. Percent survival (Y axis) over time (X axis) is shown for mice treated with the indicated TCD mRNA constructs or controls.

FIG. 5 is a graph showing that T cell disruptor mRNA constructs delay mortality in a xeno-GVHD animal model. Percent survival (Y axis) over time (X axis) is shown for mice treated with the indicated TCD mRNA constructs or controls.

FIGS. 6A-6B are graphs showing that pre-activation of B cells with CpG increases the level of expression of mRNA-encoded B cell disruptors on CD20+ B cells in vitro. FIG. 6A shows results for hPBMCs preactivated for 24 hours with either IL-21, CpG or anti-CD40. FIG. 6B shows the results for hPBMCs preactivated for 24 hours or 72 hours with CpG.

FIG. 7 is a graph showing that B cell disruptor mRNAs expressed in human B cells show a dose-dependent effect in vitro. Results are shown for human PBMCs preactivated with medium or CpG for 72 hours and treated with either 5 μM or 1 μM LNP-encapsulated BCD mRNA for 24 hours.

FIGS. 8A-8I are graphs showing that B cell disruptor mRNAs inhibit secretion of hIgM, IL-6 and IL-10 by B cells in vitro. FIGS. 8A-8C show the results for treatment of cells with 5 μM mRNA. FIG. 8D-8F show the results for treatment of cells with 1 μM mRNA. FIGS. 8G-8I show the results for treatment of cells with 200 nM mRNA. FIGS. 8A, 8D and 8G show the results for secretion of hIgM. FIGS. 8B, 8E and 8H show the results for secretion of IL-6.

FIGS. 8C, 8F and 8I show the results for secretion of IL-10.

FIGS. 9A-9B are graphs showing that B cell disruptor mRNAs reduce phosphorylation on Syk on human PBMCs or B cells. FIG. 9A shows the results for resting human PBMCs.

FIG. 9B shows the results for active B cells.

FIGS. 10A-10B are graphs showing that B cell disruptor mRNAs reduce hIgM and hIgG secretion in vivo in an NSG mouse model. FIG. 10A shows the results for hIgM at day 2 and day 7 post cell administration. FIG. 10B shows shows the results for hIgG at day 2 and day 7 post cell administration. Dots shown represent the mean from duplicate samples. The p values are shown for paired Student t test; error bars represent SEM.

FIGS. 11A-11B are graphs showing that B cell disruptor mRNAs reduce hIgM and hIgG secretion in vivo in an NSG mouse model. FIG. 11A shows the results for hIgM on days 2-15 post cell administration. FIG. 11B shows the results for hIgG on days 2-15 post cell administration. Dots shown represent the mean from 8 mice per group; error bars represent SEM.

FIGS. 12A-12B are graphs showing that B cell disruptor mRNAs reduce hIgM and hIgG secretion in vivo in an NSG mouse model. FIG. 12A shows the results for hIgM levels measured on days 2, 4, 7, 9 and 15 post cell administration. FIG. 12B shows the results for hIgG levels on days 2, 4, 7, 9 and 15 post cell administration.

FIGS. 13A-13B are graphs showing that B cell disruptor mRNAs suppress anti-TTd hIgG accumulation in vivo in an NSG mouse model following antigen challenge. FIG. 13A shows the results for anti-TTd hIgG on days 2-15 post cell administration. FIG. 13B shows the results for total serum hIgG on days 2-15 post cell administration. Dots shown represent the mean from 8 mice per group; error bars represent SEM.

FIG. 14 provides graphs showing that B cell disruptor mRNAs suppress anti-TTd hIgG accumulation in vivo in an NSG mouse model following antigenic challenge, the results for anti-TTd hIgG levels measured on days 2, 4, 7, 9 and 15 post cell administration.

FIGS. 15A-15B are graphs showing that murine B cell disruptor mRNAs reduce IgG secretion in vitro in activated rat B cells. FIG. 15A shows the results for IgG secretion on activated rat B cells. FIG. 15B shows shows the results for IgG secretion on resting rat B cells.

FIGS. 16A-16B are graphs showing that murine B cell disruptor mRNAs reduce IgM secretion in vitro in activated rat B cells. FIG. 16A shows the results for IgM secretion on activated rat B cells. FIG. 16B shows shows the results for IgM secretion on resting rat B cells.

FIGS. 17A-17B are graphs showing that murine B cell disruptor mRNAs reduce IL-10 secretion in vitro in activated rat B cells. FIG. 17A shows the results for IL-10 secretion on activated rat B cells. FIG. 17B shows shows the results for IL-10 secretion on resting rat B cells.

FIG. 18 is a graph showing that immune cell disruptor mRNA constructs inhibit collagen-induced arthritis (CIA) in an in vivo animal model. Results show aggregate CIA scores over time for rats treated with the indicated treatments.

FIG. 19 is a bar graph showing that immune cell disruptor mRNA constructs inhibit anti-Collagen Type II serum antibodies in a collagen-induced arthritis (CIA) animal model. Results show serum antibody levels as determined by ELISA.

FIG. 20 is a bar graph showing inhibition of reporter gene (SEAP) expression by transfection of Ramos-blue cells with the indicated immune cell disruptor mRNA constructs.

FIG. 21 is a bar graph showing that immune cell disruptor mRNA constructs suppress IgM secretion by human peripheral blood mononuclear cells (PBMCs).

FIG. 22 is a bar graph showing that immune cell disruptor mRNA constructs suppress IL-6 secretion by human peripheral blood mononuclear cells (PBMCs).

FIG. 23 is a bar graph showing that immune cell disruptor mRNA constructs suppress IL-10 secretion by human peripheral blood mononuclear cells (PBMCs).

FIG. 24 is a bar graph showing that immune cell disruptor mRNA constructs suppress IgG secretion in human class-switched B cells.

### DETAILED DESCRIPTION

The disclosure provides polynucleotide constructs, including mRNAs and modified mRNAs, that encode a polypeptide that inhibits immune cell activity when expressed intracellularly in the immune cell. In some embodiments, the encoded polypeptide is a chimeric polypeptide that interacts with at least one cellular component of the immune cell and disrupts (i.e., alters or inhibits) the normal signal transduction pathways within the cell that lead to activation of the cell, thereby inhibiting activity of the immune cell, for example in response to antigenic stimulation. In some embodiments, the encoded chimeric polypeptide comprises at least two portions (i.e., domains or motifs), a first portion that mediates interaction (e.g., binding or association) of the chimeric polypeptide with at least one cellular component of the immune cell, and a second portion that mediates disruption of normal signal transduction in the immune cell. Accordingly, these constructs are referred to herein as immune cell disruptor constructs.

In some embodiments, the immune cell disruptor constructs of the disclosure are advantageous in that they mediate inhibition of immune cell activity, thereby inhibiting immune responses in a subject, without causing substantial immune cell depletion. Moreover, the level of expression of a polynucleotide (e.g., mRNA) encoding an immune cell disruptor can be controlled in the target cells as they exhibit dose-dependent inhibition, thereby allowing for control of the level of inhibition desired. Still further, since the immune cell disruptors can be expressed in immune cells in a transient and controllable manner, they may avoid negative side effects observed with long-term systemic immunosuppression using non-specific agents.

### Immune Cell Disruptor Polynucleotides

One aspect of the disclosure pertains to polynucleotides that encode a polypeptide that inhibits immune cell activity when expressed in the immune cell through disruption of the normal signaling transduction pathways of the immune cell. Such polynucleotides, and the encoded polypeptides, are referred to herein as immune cell disruptor (ICD) constructs. In one embodiment, the immune cell is a T cell. In another embodiment, the immune cell is a B cell. In another embodiment, the immune cell is an NK cell. In another embodiment, the immune cell is a dendritic cell. In another embodiment, the immune cell is a macrophage.

The polynucleotides of the disclosure are typically messenger RNAs (mRNAs), although polynucleotides that are DNA molecules are also encompassed. mRNA constructs can comprise one or modified nucleotides, referred to herein as modified mRNAs (mmRNAs). In addition to the coding region encoding the chimeric polypeptide, the ICD constructs can include non-coding elements for regulating expression of the encoded polypeptide. For example, mRNA constructs typically include at least a 5′UTR, a 3′ UTR and a polyA tail in addition to the coding region. DNA constructs typically include promoter and enhancer elements in addition to the coding region.

The chimeric polypeptide encoded by the ICD construct comprises at least two portions (i.e., domains or motifs), a first portion that mediates association of the chimeric polypeptide with at least one membrane or signaling complex component of an immune cell (also referred to herein as the “association domain”, or AD) and a second portion that mediates the inhibitory effect of the immune cell disruptor construct, through disrupting normal signal transduction in the immune cell (also referred to herein as the “inhibitory domain” or ID). In one embodiment, the AD is at the N-terminal end of the chimeric polypeptide and the ID is at the C-terminal end. In another embodiment, the ID is at the N-terminal end of the chimeric polypeptide and the AD is at the C-terminal end of the chimeric polypeptide. In certain embodiments, the AD and the ID are separated by a linker polypeptide. Suitable linker polypeptides for increasing the distance between two protein domains are known in the art. In one embodiment, the linker has the sequence (GGGGS)ₙ, wherein n=1-4 (SEQ ID NO: 188). In another embodiment, there is no linker separating the AD and the ID. In certain embodiments, the AD or the ID comprises a signal sequence. In one embodiment, the signal sequence is the native signal sequence from the protein from which the AD or ID is derived. In another embodiment, the signal sequence is a heterologous signal sequence derived from a different protein than the protein from which the AD or ID is derived.

T Cell Disruptor Constructs

In one embodiment, an immune cell disruptor polynucleotide of the disclosure is a T cell disruptor (TCD) construct that inhibits the activity of a T cell when expressed intracellularly in the T cell. Inhibiting T cell activity can result in, for example, decreased T cell proliferation (e.g., decreased proliferation in response to antigenic stimulation), decreased T cell cytokine production (e.g., decreased production of TNFα and/or IFNγ) and/or inhibition of other effector functions of T cells (e.g., T helper cell activity, cytotoxic T cell activity).

A TCD polynucleotide construct encodes a chimeric polypeptide that associates with at least one component of a T cell and disrupts normal signal transduction activity in the T cell. By interfering with (i.e., disrupting, altering, inhibiting) the normal signal transduction activity in the T cell, a TCD polypeptide can increase the T cell activation threshold such that greater stimulation is necessary for the T cell to respond, thereby resulting in inhibition of T cell activity in the presence of the TCD as compared to the level of activity in the absence of the TCD.

A TCD polypeptide is a chimeric polypeptide comprising at least two portions (i.e., domains or motifs), a first portion that mediates association of the chimeric polypeptide with at least one membrane or signaling complex component of the T cell (the “association domain” or AD) and a second portion that mediates the inhibitory effect of the TCD, through disrupting normal signal transduction in the T cell (the “inhibitory domain” or ID).

Antigen-specific T cell activation is mediated through the T cell receptor (TCR) complex. The TCR complex is composed of TCR α and β chains complexed with CD3δ/ε, CD3γ/ε and ζ/ζ signaling molecules. The co-receptors CD4 (on helper T cells) and CD8 (on cytotoxic T cells) also assist signaling from the TCR complex. When the TCR is engaged by antigen presented by MHC, the tyrosine kinase Lck, which is associated with the cytoplasmic tails of CD4 and CD8, phosphorylates the intracellular chains of CD3 and chains of the TCR complex, thereby allowing another cytoplasmic tyrosine kinase, ZAP-70, to bind to them. Lck then phosphorylates and activates ZAP-70, which in turn phosphorylates another molecule in the signaling cascade, LAT (also known as Linker of Activated T cells). LAT serves as a docking site for a number of other proteins involved in the TCR signaling cascade, including PLCγ, SOS, GADS, GRB2, SLP76, ITK, VAV, NCK, ADAP and PI3K.

Furthermore, upon T cell activation, a fraction of kinase-active Lck translocates from outside lipid rafts in the cell membrane to inside lipid rafts, where it interacts with and activates the kinase Fyn residing in the lipid rafts. Fyn is then involved in further downstream signaling activation.

In addition to receptor-associated signaling subunits, T cells also contain transmembrane adaptor proteins (TRAPs), which are not directly associated with a receptor but still are involved directly or indirectly in the regulation of receptor signaling. One example of such a TRAP is PAG (phosphoprotein associated with glycosphingolipid microdomains), also known as Csk-binding protein (Cbp). Additionally, T cells contain other membrane-associated proteins that interact with T cell signaling components, such as membrane-associated Src.

Important components in the regulation of the TCR-mediated signaling cascade are kinases and phosphatases that inhibit activator components of the signaling cascade. For example, the cytosolic kinase Csk (C-terminal Src kinase) is a negative regulator of Lck through phosphorylation on the inhibitory tyrosine 505. Lck is also inhibited by the phosphatase SHP-1 (also known as Src homology region 2 domain-containing phosphatase-1 and tyrosine-protein phosphatase non-receptor type 6, or PTPN6), whose phosphatase activity dephosphorylates Lck on the activating tyrosine 394. The phosphatase PTPN22 also dephosphorylates Lck on the activating tyrosine 394, as well as ZAP-70 on the activating tyrosine 493. The phosphatases PTPN1 and PTEN are also involved in inhibiting TCR-mediated signaling, for example through dephosphorylating the intracellular signaling molecules Grb2 and PIP3, respectively. Moreover, the SHIP1 phosphatase is also an inhibitor of intracellular signaling through negatively regulating the PI3K signaling pathway.

Furthermore, the GTPase KRAS plays a role in T cell signaling. KRAS is typically tethered to cell membranes because of the presence of an isoprene group in its C-terminus.

Other important components in the regulation of the TCR-mediated signaling cascade are inhibitory receptors, examples of which include CTLA4 and LAIR1. These are both surface receptors that are members of the immunoglobulin superfamily that delivery inhibitory signals to T cells. LAIR1 contains two ITIMs in its cytoplasmic tail, whereas CTLA4 contains an ITIM-like motif in its cytoplasmic tail.

TCD Association Domains

The association domain (AD) of a T cell disruptor construct of the disclosure can be derived from any of a number of different types of T cell components that interact with other components within the T cell, including membrane receptor-associated components, membrane receptor components, transmembrane-associated components or intracellular-associated components.

Non-limiting examples of membrane receptor-associated T cell components from which the association domain can be derived include Lck (which associates with the CD4 and CD8 receptors) and ZAP-70 (which associates with CD3).

Accordingly, in one embodiment, the AD is derived from a Lck protein, such as a CD4-binding or CD8-binding portion of a Lck protein. In one embodiment, the AD is an N-terminal portion of a Lck protein (e.g., human Lck), such as amino acid residues 1-50 of human Lck (e.g., having the amino acid sequence shown in SEQ ID NO: 13) or amino acid residues 1-72 of human Lck (e.g., having the amino acid sequence shown in SEQ ID NO: 20). In another embodiment, the AD is derived from a Lck protein and comprises SH2 and SH3 domains of Lck, such as human Lck SH2-SH3 domains (e.g., having the amino acid sequence shown in SEQ ID NO: 7).

In another embodiment, the AD is derived from a ZAP-70 protein (e.g., human ZAP-70 protein), such as a CD3-binding portion of ZAP-70. In one embodiment, the AD comprises a portion of ZAP-70 that contains at least one SH2 domain. In one embodiment, the AD comprises a portion of ZAP-70 (e.g., human ZAP-70) that contains the N-terminal SH2 domain, interdomain A (I-A), the C-terminal SH2 domain and interdomain B (I-B) (e.g., having the amino acid sequence shown in SEQ ID NO: 1). In one embodiment, the AD comprises a portion of ZAP-70 (e.g., human ZAP-70) that contains the N-terminal SH2 domain, interdomain A (I-A), the C-terminal SH2 domain and interdomain B (I-B), further comprising the following mutations in the I-B domain: Y292A/Y315A/Y319A (e.g., having the amino acid sequence shown in SEQ ID NO: 2). In one embodiment, the AD comprises a portion of ZAP-70 (e.g., human ZAP-70) that contains the N-terminal SH2 domain, interdomain A (I-A), the C-terminal SH2 domain (e.g., having the amino acid sequence shown in SEQ ID NO: 3). In one embodiment, the AD comprises a portion of ZAP-70 (e.g., human ZAP-70) that contains the N-terminal SH2 domain and the C-terminal SH2 domain, optionally separated by a linker polypeptide (e.g, a G45 linker polypeptide) (e.g., having the amino acid sequence shown in SEQ ID NO: 4).

Non-limiting examples of membrane-associated T cell components from which the association domain can be derived include the Fyn, Src and KRAS proteins.

Accordingly, in one embodiment, the AD is derived from a Fyn protein (e.g., human Fyn), such as a membrane-binding portion thereof. In one embodiment, the AD comprises an N-terminal portion of Fyn, such as amino acid residues 1-50 of human Fyn (e.g., having the amino acid sequence shown in SEQ ID NO: 14).

In another embodiment, the AD is derived from a Src protein (e.g., human Src), such as a membrane-binding portion thereof. In one embodiment, the AD comprises an N-terminal portion of Src, such as amino acid residues 1-10 of human Src (e.g., having the amino acid sequence shown in SEQ ID NO: 15).

In another embodiment, the AD is derived from a KRAS protein (e.g., human KRAS), such as a membrane-binding portion thereof. In one embodiment, the AD comprises a C-terminal portion of KRAS, such as amino acid residues 166-186 of human KRAS (e.g., having the amino acid sequence shown in SEQ ID NO: 19).

A non-limiting example of a transmembrane-associated T cell component from which the association domain can be derived is the PAG protein. Accordingly, in one embodiment, the AD is derived from a PAG protein (e.g., human PAG), such as a membrane-binding portion thereof. In one embodiment, the AD comprises an N-terminal portion of PAG, such as amino acid residues 1-47 of human PAG (e.g., having the amino acid sequence shown in SEQ ID NO: 12).

Non-limiting examples of intracellular-associated T cell components from which the association domain can be derived include the LAT, Grb2, Grap, PI3K, PLCγ1, GADS, ADAP, NCK, VAV, SOS, ITK and SLP76 proteins.

Accordingly, in one embodiment, the AD is derived from a LAT protein (e.g., human LAT), such as the full-length LAT protein or a ZAP-70-binding portion thereof. In one embodiment, the AD comprises a full-length LAT protein, such as full-length human LAT (e.g., having the amino acid sequence shown in SEQ ID NO: 8). In one embodiment, the AD comprises an N-terminal portion of LAT, such as amino acid residues 1-160 of human LAT (e.g., having the amino acid sequence shown in SEQ ID NO: 9) or amino acid residues 1-38 of human LAT (e.g., having the amino acid sequence shown in SEQ ID NO: 10) or amino acid residues 1-33 of human LAT (e.g., having the amino acid sequence shown in SEQ ID NO: 11) or amino acid residues 1-38 of mouse LAT (e.g., having the amino acid sequence shown in SEQ ID NO: 16).

In another embodiment, the AD is derived from a Grb2 protein (e.g., human Grb2), such as a LAT-binding portion thereof. In one embodiment, the AD comprises a portion of Grb2 containing an SH2 domain, such as amino acid residues 59-152 of human Grb2 (e.g., having the amino acid sequence shown in SEQ ID NO: 5).

In another embodiment, the AD is derived from a Grap protein (e.g., human Grap), such as a LAT-binding portion thereof. In one embodiment, the AD comprises a portion of Grap containing an SH2 domain, such as amino acid residues 60-154 of human Grap (e.g., having the amino acid sequence shown in SEQ ID NO: 6).

In another embodiment, the AD is derived from a PI3K protein, such as a PI3K.p85a protein (also known as phosphatidylinositol 3-kinase regulatory subunit alpha) (e.g., human PI3K.p85a). In one embodiment, the AD comprises a portion of PI3K.p85α in which an internal region containing an iSH2 domain has been deleted, such as amino acid residues 1-111, 303-724 of human PI3K.p85α, wherein residues 112-302 have been deleted (e.g., a portion having the amino acid sequence shown in SEQ ID NO: 17).

In another embodiment, the AD is derived from a PLCγ1 protein, (e.g., human PLCγ1), such as a LAT-binding portion thereof. In one embodiment, the AD comprises a portion of PLCγ1 containing SH2 and SH3 domains, such as amino acid residues 550-850 of human PLCγ1 (e.g., having the amino acid sequence shown in SEQ ID NO: 18).

In one embodiment, the AD of the T cell disruptor has an amino acid sequence selected from the group consisting of the sequences shown in SEQ ID NOs: 1-20.

TCD Inhibitory Domains

The inhibitory domain of a T cell disruptor construct of the disclosure can be derived from any of a number of different T cell components involved in signal transduction and subsequent T cell activation. For example, in one embodiment, the inhibitory domain functions to reverse ITIM/ITAM polarity, to thereby favor inhibitory signaling. In another embodiment, the inhibitory domain functions to recruit regulatory Csk to thereby promote inhibitory signaling. In another embodiment, the inhibitory domain functions to recruit a regulatory phosphatase to thereby promote inhibitory signaling. In yet another embodiment, the inhibitory domain alters (e.g., inhibits, downregulates) PI3K signaling to thereby inhibit T cell activity. To mediate its inhibitory function, in one embodiment the inhibitory domain comprises one or more phosphatase domains. In another embodiment, the inhibitory domain comprises one or more kinase domains. In another embodiment, the inhibitory domain comprises one or more ITIMs.

Accordingly, in one embodiment, the inhibitory domain (ID) of the T cell disruptor is derived from a SHP1 protein (also known as SH2-containing phosphatase-1 and tyrosine-protein phosphatase non-receptor type 6). (e.g., a human SHP1 protein) and comprises a SHP1 phosphatase domain. For example, in one embodiment, the ID comprises amino acids 244-515 of human SHP1 (e.g., having the amino acid sequence shown in SEQ ID NO: 21). In another embodiment, the ID comprises amino acids 2-515 of human SHP1 (e.g., having the amino acid sequence shown in SEQ ID NO: 27).

In another embodiment, the inhibitory domain (ID) of the T cell disruptor is derived from a SHIP1 protein (also known as SH2-containing inositol phosphatase-1) (e.g., a human SHIP1 protein) and comprises a SHIP1 phosphatase domain. For example, in one embodiment, the ID comprises amino acids 111-910 of human SHIP1 (e.g., having the amino acid sequence shown in SEQ ID NO: 31).

In another embodiment, the inhibitory domain (ID) of the T cell disruptor is derived from a PTPN22 protein (also known as protein tyrosine phosphatase, non-receptor type 22) (e.g., a human PTPN22 protein) and comprises a PTPN22 phosphatase domain. In one embodiment, the ID comprises an N-terminal portion of PTPN22, such as amino acid residues 1-290 of human PTPN22 (e.g., having the amino acid sequence shown in SEQ ID NO: 32). In another embodiment, the ID comprises an N-terminal portion of PTPN22 and further comprises a mutation at a serine residue within the catalytic domain that is involved in regulating PTPN22 activity, such as amino acid residues 1-290 of human PTPN22 with a S35A mutation (e.g., having the amino acid sequence shown in SEQ ID NO: 33) or amino acid residues 24-289 of human PTPN22 with a S35A mutation (e.g., having the amino acid sequence shown in SEQ ID NO: 34).

In another embodiment, the inhibitory domain (ID) of the T cell disruptor is derived from a PTPN1 protein (also known as protein tyrosine phosphatase, non-receptor type 1) (e.g., a human PTPN1 protein) and comprises a PTPN1 phosphatase domain. In one embodiment, the ID comprises an N-terminal portion of PTPN1, such as amino acid residues 3-277 of human PTPN1 (e.g., having the amino acid sequence shown in SEQ ID NO: 29).

In another embodiment, the inhibitory domain (ID) of the T cell disruptor is derived from a PTEN protein (e.g., a human PTEN protein) and comprises a PTEN phosphatase domain. In one embodiment, the ID comprises a mutated PTEN polypeptide. In one embodiment, the ID comprises a PTEN polypeptide comprising one or more lysine to glutamic acid mutations, such as amino acid residues 1-350 of human PTEN having K13E and K289E mutations (e.g., having the amino acid sequence shown in SEQ ID NO: 30).

In another embodiment, the inhibitory domain (ID) of the T cell disruptor is derived from a Csk protein (e.g., a human Csk protein) and comprises a Csk kinase domain. For example, in one embodiment, the ID comprises amino acid residues 195-449 of human Csk (e.g., having the amino acid sequence shown in SEQ ID NO: 26). In another embodiment, the ID comprises a constitutively active form of Csk, such as the full-length human Csk protein having the following mutations: W47A/R107K/E154A (e.g., having the amino acid sequence shown in SEQ ID NO: 25).

In another embodiment, the inhibitory domain (ID) of the T cell disruptor is derived from a LAIR1 protein (also known as leukocyte-associated immunoglobulin-like receptor 1)(e.g., a human LAIR1 protein) and comprises at least one ITIM motif. In one embodiment, the ID comprises ITIM1 of LAIR1 (located at amino acid residues 249-254 of human LAIR1). In another embodiment, the ID comprises ITIM2 of LAIR1 (located at amino acid residues 279-284 of human LAIR1). In another embodiment, the ID comprises both ITIM1 and ITIM2 of LAIR. For example, in one embodiment, the ID comprises amino acid residues 187-287 of human LAIR1 (e.g., having the amino acid sequence shown in SEQ ID NO: 24). In another embodiment, the ID comprises a polypeptide into which the LAIR1 ITIM1 and/or ITIM2 sequences have been inserted. For example, in one embodiment, the ID comprises a LAT polypeptide in which the LAIR1 ITIM1 motif replaces one or more alanine-containing regions (e.g., three regions) within the C-terminal region of LAT (e.g., having the amino acid sequence shown in SEQ ID NO: 22). In another embodiment, the ID comprises a LAT polypeptide in which the LAIR1 ITIM2 motif replaces one or more alanine-containing regions (e.g., three regions) within the C-terminal region of LAT (e.g., having the amino acid sequence shown in SEQ ID NO: 23).

In another embodiment, the inhibitory domain (ID) of the T cell disruptor is derived from a CTLA4 protein (e.g., a human CTLA4 protein) and comprises the ITIM-like motif of CTLA4. In one embodiment, the ID comprises a C-terminal portion of CTLA4. For example, in one embodiment, the ID comprise amino acid residues 182-223 of human CTLA4 (e.g., having the amino acid sequence shown in SEQ ID NO: 28).

In one embodiment, the ID of the T cell disruptor has an amino acid sequence selected from the group consisting of the sequences shown in SEQ ID NOs: 21-34.

The preparation of representative examples of T cell disruptor constructs are described in detail in Example 1. The ability of the constructs to inhibit T cell activity in vitro, including inhibiting T cell proliferation and cytokine secretion are described in Examples 2 and 3, respectively. The ability of the constructs to inhibit T cell activity in vivo, including delaying mortality in a GVHD model, is described in Example 4.

In one embodiment, the disclosure provides a TCD construct comprising an association domain derived from ZAP-70 and an inhibitory domain derived from SHP1. Representative nucleotide sequences such constructs are shown in SEQ ID NOs: 35-38. Representative amino acid sequences for such constructs are shown in SEQ ID NOs: 81-84.

In one embodiment, the disclosure provides a TCD construct comprising an association domain derived from Grb2 and an inhibitory domain derived from SHP1. A representative nucleotide sequence for such a construct is shown in SEQ ID NO: 39. A representative amino acid sequence for such a construct is shown in SEQ ID NO: 85.

In one embodiment, the disclosure provides a TCD construct comprising an association domain derived from Grap and an inhibitory domain derived from SHP1. A representative nucleotide sequence for such a construct is shown in SEQ ID NO: 40. A representative amino acid sequence for such a construct is shown in SEQ ID NO: 86.

In one embodiment, the disclosure provides a TCD construct comprising an association domain derived from Lck and an inhibitory domain derived from SHP1. Representative nucleotide sequences for such constructs are shown in SEQ ID NOs: 41, 60 and 65. Representative amino acid sequences for such constructs are shown in SEQ ID NOs: 87, 106 and 111.

In one embodiment, the disclosure provides a TCD construct comprising an association domain derived from Lck and an inhibitory domain derived from Csk. Representative nucleotide sequences for such constructs are shown in SEQ ID NOs: 50 and 55. Representative amino acid sequences for such constructs are shown in SEQ ID NOs: 96 and 101.

In one embodiment, the disclosure provides a TCD construct comprising an association domain derived from Lck and an inhibitory domain derived from PTPTN22. A representative nucleotide sequence for such a construct is shown in SEQ ID NO: 80. A representative amino acid sequence for such a construct is shown in SEQ ID NO: 126.

In one embodiment, the disclosure provides a TCD construct comprising an association domain derived from LAT and an inhibitory domain derived from LAIR1. Representative nucleotide sequences for such constructs are shown in SEQ ID NOs: 42-44 and 47. Representative amino acid sequences for such constructs are shown in SEQ ID NOs: 88-90 and 93.

In one embodiment, the disclosure provides a TCD construct comprising an association domain derived from LAT and an inhibitory domain derived from SHP1. Representative nucleotide sequences for such constructs are shown in SEQ ID NOs: 45, 46, 58 and 63. Representative amino acid sequences for such constructs are shown in SEQ ID NOs: 0.91, 92, 104 and 109.

In one embodiment, the disclosure provides a TCD construct comprising an association domain derived from LAT and an inhibitory domain derived from Csk. Representative nucleotide sequences for such constructs are shown in SEQ ID NOs: 48 and 53. Representative amino acid sequences for such constructs are shown in SEQ ID NOs: 94 and 99.

In one embodiment, the disclosure provides a TCD construct comprising an association domain derived from LAT and an inhibitory domain derived from CTLA4. Representative nucleotide sequences for such constructs are shown in SEQ ID NOs: 68 and 69. Representative amino acid sequences for such constructs are shown in SEQ ID NOs: 114 and 115.

In one embodiment, the disclosure provides a TCD construct comprising an association domain derived from LAT and an inhibitory domain derived from PTPN1. A representative nucleotide sequence for such a construct is shown in SEQ ID NO: 70. A representative amino acid sequence for such a construct is shown in SEQ ID NO: 116.

In one embodiment, the disclosure provides a TCD construct comprising an association domain derived from PAG and an inhibitory domain derived from SHP1. Representative nucleotide sequences for such constructs are shown in SEQ ID NOs: 59 and 64. Representative amino acid sequences for such constructs are shown in SEQ ID NOs: 105 and 110.

In one embodiment, the disclosure provides a TCD construct comprising an association domain derived from PAG and an inhibitory domain derived from Csk. Representative nucleotide sequences for such constructs are shown in SEQ ID NOs: 49 and 54. Representative amino acid sequences for such constructs are shown in SEQ ID NOs: 95 and 100.

In one embodiment, the disclosure provides a TCD construct comprising an association domain derived from Fyn and an inhibitory domain derived from SHP1. Representative nucleotide sequences for such constructs are shown in SEQ ID NOs: 61 and 66. Representative amino acid sequences for such constructs are shown in SEQ ID NOs: 107 and 112.

In one embodiment, the disclosure provides a TCD construct comprising an association domain derived from Fyn and an inhibitory domain derived from Csk. Representative nucleotide sequences for such constructs are shown in SEQ ID NOs: 52 and 57. Representative amino acid sequences for such constructs are shown in SEQ ID NOs: 98 and 103.

In one embodiment, the disclosure provides a TCD construct comprising an association domain derived from Src and an inhibitory domain derived from SHP1. Representative nucleotide sequences for such constructs are shown in SEQ ID NOs: 62 and 67. Representative amino acid sequences for such constructs are shown in SEQ ID NOs: 108 and 113.

In one embodiment, the disclosure provides a TCD construct comprising an association domain derived from Src and an inhibitory domain derived from Csk. Representative nucleotide sequences for such constructs are shown in SEQ ID NOs: 51 and 56. Representative amino acid sequences for such constructs are shown in SEQ ID NOs: 97 and 102.

In one embodiment, the disclosure provides a TCD construct comprising an association domain derived from PI3K.p85α and an inhibitory domain derived from PTEN. A representative nucleotide sequence for such a construct is shown in SEQ ID NO: 71. A representative amino acid sequence for such a construct is shown in SEQ ID NO: 117.

In one embodiment, the disclosure provides a TCD construct comprising an association domain derived from PI3K.p85α and an inhibitory domain derived from SHIP1. A representative nucleotide sequence for such a construct is shown in SEQ ID NO: 72. A representative amino acid sequence for such a construct is shown in SEQ ID NO: 118.

In one embodiment, the disclosure provides a TCD construct comprising an association domain derived from PLCγ1 and an inhibitory domain derived from SHIP1. Representative nucleotide sequences for such constructs are shown in SEQ ID NOs: 73 and 74. Representative amino acid sequences for such constructs are shown in SEQ ID NOs: 119 and 120.

In one embodiment, the disclosure provides a TCD construct comprising an association domain derived from PLCγ1 and an inhibitory domain derived from PTEN. Representative nucleotide sequences for such constructs are shown in SEQ ID NOs: 75 and 76. Representative amino acid sequences for such constructs are shown in SEQ ID NOs: 121 and 122.

In one embodiment, the disclosure provides a TCD construct comprising an association domain derived from KRAS and an inhibitory domain derived from PTEN. A representative nucleotide sequence for such a construct is shown in SEQ ID NO: 77. A representative amino acid sequence for such a construct is shown in SEQ ID NO: 123.

In one embodiment, the disclosure provides a TCD construct comprising an association domain derived from KRAS and an inhibitory domain derived from PTPN22. Representative nucleotide sequences for such constructs are shown in SEQ ID NOs: 78 and 79. Representative amino acid sequences for such constructs are shown in SEQ ID NOs: 124 and 125.

In one embodiment, the disclosure provides a TCD construct comprising an inhibitory domain derived from SHP1 and an association domain derived from a protein selected from the group consisting of ZAP-70, Grb2, Grap, Lck, LAT, PAG, Fyn, Src, PI3K.p85α and PLCγ1. Representative nucleotide sequences for such constructs are shown in SEQ ID NOs: 35-41, 45, 46, 58-67 and 72-74. Representative amino acid sequences for such constructs are shown in SEQ ID NOs: 81-87, 91, 92, 104-113 and 118-120.

In one embodiment, the disclosure provides a TCD construct comprising an inhibitory domain derived from Csk and an association domain derived from a protein selected from the group consisting of LAT, PAG, Lck, Fyn, Src and PLCγ1. Representative nucleotide sequences for such constructs are shown in SEQ ID NOs: 48-57. Representative amino acid sequences for such constructs are shown in SEQ ID NOs: 94-103

In one embodiment, the disclosure provides a TCD construct comprising an inhibitory domain derived from PTEN and an association domain derived from a protein selected from the group consisting of PI3K.p85α and PLCγ1. Representative nucleotide sequences for such constructs are shown in SEQ ID NOs: 71, 75 and 76. Representative amino acid sequences for such constructs are shown in SEQ ID NOs: 117, 121 and 122.

In one embodiment, the disclosure provides a TCD construct comprising an inhibitory domain derived from PTPN22 and an association domain derived from a protein selected from the group consisting of KRAS and Lck. Representative nucleotide sequences for such constructs are shown in SEQ ID NOs: 78-80. Representative amino acid sequences for such constructs are shown in SEQ ID NOs: 124-126.

B Cell Disruptor Constructs

In one embodiment, an immune cell disruptor polynucleotide of the disclosure is a B cell disruptor (BCD) construct that inhibits the activity of a B cell when expressed intracellularly in the B cell. Inhibiting B cell activity can result in, for example, decreased B cell proliferation (e.g., decreased proliferation in response to antigenic stimulation), decreased B cell cytokine production (e.g., decreased production of IL-6 and/or and IL-10) and/or decreased immunoglobulin production (e.g., decreased IgM and/or IgG production).

A BCD polynucleotide construct encodes a chimeric polypeptide that associates with at least one component of a B cell and disrupts normal signal transduction activity in the B cell. By interfering with (i.e., disrupting, altering, inhibiting) the normal signal transduction activity in the B cell, a BCD polypeptide can increase the B cell activation threshold such that greater stimulation is necessary for the B cell to respond, thereby resulting in inhibition of B cell activity in the presence of the BCD as compared to the level of activity in the absence of the BCD.

A BCD polypeptide is a chimeric polypeptide comprising at least two portions (i.e., domains or motifs), a first portion that mediates association of the chimeric polypeptide with at least one membrane or signaling complex component of the B cell (the “association domain”) and a second portion that mediates the inhibitory effect of the BCD, through disrupting normal signal transduction in the B cell (the “inhibitory domain”).

Antigen-specific B cell activation is mediated through the B cell receptor (BCR) complex. The BCR complex is composed of surface membrane-bound immunoglobulin light and heavy chains and the signal-transducing CD79a/CD79b heterodimer. The cytoplasmic tails of CD79a and CD79b each contain an immunoreceptor tyrosine-based activation motif (ITAM) with two conserved tyrosines. For normal signaling through the BCR, following antigen ligation of the cell surface BCR, the two tyrosine residues in the ITAMs are phosphorylated by the src-family kinase Lyn, which attracts and activates spleen tyrosine kinase (Syk). The resulting ITAM/Syk complex amplifies the BCR signal and connects the BCR to several downstream signaling pathways, leading to the activation, proliferation, and differentiation of B cells.

Another important signaling hub in B cells is the CD19 co-receptor, which associates with CD81 and CD21 on the cell surface, and serves as an amplifier or propagator of BCR signaling. CD19 has a long cytoplasmic tail with 9 tyrosine sites. Most of them are phosphorylated by Lyn. Once phosphorylated, these tyrosines serve as binding partners for the adaptor proteins PI3K and PLCy, leading to PI3K signaling and cytoskeleton rearrangements. On resting B cells, mature B cells co-express BCR and CD19 but the proteins reside in different protein islands on the cell membrane. Upon activation of the B cells, the CD19 complex moves to the open BCR island and sequentially engages Syk and gains access to BCR-ITAM signaling, thereby amplifying or propagating BCR-mediated signaling.

CD22 is another regulator of BCR signaling on conventional B cells (B-2 cells) and has an inhibitory function. CD22 is a sugar binding transmembrane protein, with its N-terminus binding to sialic acid and its C-terminal cytoplasmic domain containing three immunoreceptor tyrosine-based inhibitory motifs (ITIMs). Normally, CD22 and the BCR are separated from each other on the B cell surface. Following antigen binding to the BCR, CD22 molecules are recruited to the BCR island, leading to phosphorylation of the ITIMs by Lyn. The phosphorylated ITIMs then recruit the phosphatase SHP-1 to the BCR, which strongly blunts BCR signaling. Thus, CD19 and CD22 recruite different downstream proteins and provide a stimulatory/inhibitory balance to regulate BCR activation.

BCD Association Domains

The association domain of a B cell disruptor construct of the disclosure can be derived from any of a number of different types of B cell components that interact with other components within the B cell, including membrane receptor-associated components, membrane receptor components, transmembrane-associated components or intracellular-associated components.

Non-limiting examples of membrane receptor-associated B cell components from which the association domain can be derived include the CD79a and CD79b proteins. These proteins associate with the cytoplasmic region of the BCR in B cells. In one embodiment, an N-terminal portion of CD79a or CD79b is used as the AD that is capable of interacting with the BCR but which lacks the downstream activatory ITAMs. In another embodiment, the full-length CD79a or CD79b protein is used as the AD but the ITAMs are mutated, such that the AD is still capable of interacting with the BCR but is not capable of being phosphorylated by Lyn.

Accordingly, in one embodiment, the AD of the B cell disruptor is derived from a CD79a protein. In one embodiment, an N-terminal portion of CD79a (e.g., human CD79a) is used, such as amino acid residues 1-176 of human CD79a (e.g., having the amino acid sequence shown in SEQ ID NO: 128), or amino acid residues 1-170 of mouse CD79a (e.g., having the amino acid sequence shown in SEQ ID NO: 139) or amino acid residues 1-171 of rat CD79a (e.g., having the amino acid sequence shown in SEQ ID NO: 142). In another embodiment, the full-length CD79a protein is used as the AD, wherein the ITAMs have been mutated (e.g., tyrosine residues within the ITAM have been mutated, for example, to alanine). For example, in one embodiment, full-length human CD79a is used having mutations Y188A/Y199A (e.g., having the amino acid sequence shown in SEQ ID NO: 127). In one embodiment, full-length mouse CD79a is used having the mutations Y182A/Y193A (e.g., having the amino acid sequence shown in SEQ ID NO: 135).

In another embodiment, the AD of the B cell disruptor is derived from a CD79b protein. In one embodiment, an N-terminal portion of CD79b (e.g., human CD79b) is used, such as amino acid residues 1-184 of human CD79b (e.g., having the amino acid sequence shown in SEQ ID NO: 130), or amino acid residues 1-183 of mouse CD79b (e.g., having the amino acid sequence shown in SEQ ID NO: 140) or amino acid residues 1-183 of rat CD79b (e.g., having the amino acid sequence shown in SEQ ID NO: 143). In another embodiment, the full-length CD79b protein is used as the AD, wherein the ITAMs have been mutated (e.g., tyrosine residues within the ITAM have been mutated, for example, to alanine). For example, in one embodiment, full-length human CD79b is used having mutations Y196A/Y207A (e.g., having the amino acid sequence shown in SEQ ID NO: 129). In another embodiment, full-length mouse CD79b is used having the mutations Y195A/Y206A (e.g., having the amino acid sequence shown in SEQ ID NO: 136).

A non-limiting example of a membrane receptor B cell component from which the association domain can be derived is the CD19 protein. CD19 associates with CD21 and CD81 in B cells. In one embodiment, an N-terminal portion of CD19 is used as the AD that is capable of interacting with CD21 and/or CD81 but which lacks the downstream activatory ITAMs. In another embodiment, the full-length CD19 protein is used as the AD but the ITAMs are mutated, such that the AD is still capable of interacting with the CD21 and/or CD81 but is not capable of being phosphorylated by Lyn.

Accordingly, in one embodiment, the AD of the B cell disruptor is derived from a CD19 protein. In one embodiment, an N-terminal portion of CD19 (e.g., human CD19) is used, such as amino acid residues 1-313 of human CD19 (e.g., having the amino acid sequence shown in SEQ ID NO: 131), or amino acid residues 1-311 of mouse CD19 (e.g., having the amino acid sequence shown in SEQ ID NO: 137) or amino acid residues 1-311 of rat CD19 (e.g., having the amino acid sequence shown in SEQ ID NO: 141). In another embodiment, the full-length CD19 protein is used as the AD, wherein the ITAMs have been mutated (e.g., tyrosine residues within the ITAM have been mutated, for example, to alanine). For example, in one embodiment, full-length human CD19 is used having mutations Y378A/Y409A/Y439A/Y500A (e.g., having the amino acid sequence shown in SEQ ID NO: 132). In one embodiment, full-length mouse CD19 is used having the mutations Y376A/Y402A/Y432A/Y493A (e.g., having the amino acid sequence shown in SEQ ID NO: 138).

Another non-limiting example of a membrane receptor B cell component from which the association domain can be derived is the CD64 protein. CD64, also known as Fc-gamma receptor 1 (FcγR1), is a B cell surface receptor that binds IgG. Following IgG binding, CD64 interacts with an accessory chain known as the common γ chain (γ chain), which possesses an ITAM motif that is necessary for triggering cellular activation. Thus, in one embodiment, an N-terminal portion of CD64 is used as the AD that is capable of interacting with the B cell surface and binding IgG but which lacks the ability to interact with the γ chain. For example, in one embodiment, an N-terminal portion of human CD64 is used, such as amino acid residues 1-313 (e.g., having the amino acid sequence shown in SEQ ID NO: 133). In another embodiment, an N-terminal portion of mouse CD64 is used, such as amino acid residues 1-320 (e.g., having the amino acid sequence shown in SEQ ID NO: 134).

Another non-limiting example of a membrane receptor-associated B cell components from which the association domain can be derived is the Syk protein. For normal signaling through the BCR, following antigen ligation of the cell surface BCR, the two tyrosine residues in the ITAMs are phosphorylated by the src-family kinase Lyn, which attracts and activates spleen tyrosine kinase (Syk). The resulting ITAM/Syk complex amplifies the BCR signal and connects the BCR to several downstream signaling pathways, leading to the activation, proliferation, and differentiation of B cells. Thus, in one embodiment, Syk, or a portion thereof, is used as the AD in a BCD construct. For example, in various embodiment, a Syk polypeptide having the amino acid sequence shown in SEQ ID NO: 229, 230 or 231 can be used as the AD.

In one embodiment, the AD of the B cell disruptor is from a protein selected from the group consisting of CD79a, CD79b, CD19, CD64 and Syk. In one embodiment, the AD of the B cell disruptor is selected from the group consisting of an N-terminal portion of CD79a lacking ITAMs, an N-terminal portion of CD79b lacking ITAMs, a CD79a polypeptide having non-functional (e.g., mutated) ITAMs, a CD79b polypeptide having non-functional (e.g., mutated) ITAMs, an N-terminal portion of CD19 lacking ITAMs, a CD19 polypeptide having non-functional (e.g., mutated) ITAMs and an N-terminal portion of CD64.

In one embodiment, the AD of the B cell disruptor has an amino acid sequence selected from the group consisting of the sequences shown in SEQ ID NOs: 127-143 and 229-231.

BCD Inhibitory Domains

The inhibitory domain of a B cell disruptor construct of the disclosure can be derived from any of a number of different B cell components involved in signal transduction and subsequent B cell activation. For example, in one embodiment, the inhibitory domain functions to alter the CD19/CD22 balance in the B cells, thereby altering the balance of activatory versus inhibitory signals from those molecules to increase (e.g., promote, upregulate, stimulate) B cell inhibition. In another embodiment, the inhibitory domain functions to inhibit signaling through the BCR complex, in particular signaling mediated through CD79a/CD79b, to thereby inhibit B cell activity. In yet another embodiment, the inhibitory domain functions to alter Fc receptor activity/signaling to thereby inhibit B cell activation. In yet another embodiment, the inhibitory domain alters (e.g., inhibits, downregulates) PI3K signaling to thereby inhibit B cell activity. To mediate its inhibitory function, in one embodiment the inhibitory domain comprises one or more ITIMs. In another embodiment, the inhibitory domain comprises one or more phosphatase domains.

Accordingly, in one embodiment, the inhibitory domain of the B cell disruptor is derived from a CD22 protein (e.g., a human CD22 protein) and comprises one or more ITIMs. For example, the ID can be a C-terminal portion of a CD22 protein, which comprises three ITIMs, such as amino acid residues 580-675 of human CD22 (e.g., having the amino acid sequence shown in SEQ ID NO: 144) or amino acid residues 773-868 of mouse CD22 (e.g., having the amino acid sequence shown in SEQ ID NO: 148) or amino acid residues 757-852 of rat CD22 (e.g., having the amino acid sequence shown in SEQ ID NO: 149).

In another embodiment, the inhibitory domain of the BCD is derived from a SHP1 protein (also known as Src homology region 2 domain-containing phosphatase-1 and tyrosine-protein phosphatase non-receptor type 6). For example, the phosphatase domain of SHP1 can be used as the ID, such as amino acid residues 244-515 of human SHP1 (e.g., having the amino acid sequence shown in SEQ ID NO: 145).

In yet another embodiment, the inhibitory domain of the BCD is derived from a CD32b protein, also known as Fc-gamma receptor IIB (FcγRIIB), which carries an ITIM. For example, a C-terminal portion of CD32b that contains the ITIM can be used, such as amino acid residues 241-310 of human CD32b (e.g., having the amino acid sequence shown in SEQ ID NO: 146) or amino acid residues 241-340 of mouse CD32b (e.g., having the amino acid sequence shown in SEQ ID NO: 147).

In another embodiment, the inhibitory domain (ID) of the B cell disruptor is derived from a Csk protein (e.g., a human Csk protein) and comprises a Csk kinase domain. For example, in one embodiment, the ID comprises amino acid residues 195-449 of human Csk (e.g., having the amino acid sequence shown in SEQ ID NO: 26). In another embodiment, the ID comprises a constitutively active form of Csk, such as the full-length human Csk protein having the following mutations: W47A/R107K/E154A (e.g., having the amino acid sequence shown in SEQ ID NO: 25).

In one embodiment, the ID of the B cell disruptor is from a protein selected from the group consisting of CD22, SHP1, CD32b and Csk. In one embodiment, the ID of the B cell disruptor is selected from the group consisting of an C-terminal portion of CD22 comprising at least one ITIM, a C-terminal portion of CD32b comprising at least one ITIM and a portion of SHP1 comprising a phosphatase domain.

In one embodiment, the ID of the B cell disruptor has an amino acid sequence selected from the group consisting of the sequences shown in SEQ ID NOs: 25, 26 and 144-149.

The preparation of representative examples of B cell disruptor constructs are described in detail in Examples 5 and 11. The ability of the constructs to inhibit B cell activity in vitro, including immunoglobulin production and cytokine secretion are described in Examples 7, 9 and 12. The ability of the constructs to inhibit B cell activity in vivo, including IgM and IgG production, as well as antigen-specific antibody accumulation, is described in Examples 8 and 10.

In one embodiment, the disclosure provides a BCD construct comprising an association domain derived from CD79a and an inhibitory domain derived from CD22. Representative nucleotide sequences such constructs are shown in SEQ ID NOs: 150-151, 159, 163 and 166. Representative amino acid sequences for such constructs are shown in SEQ ID NOs: 168-169, 177, 181 and 184.

In one embodiment, the disclosure provides a BCD construct comprising an association domain derived from CD79b and an inhibitory domain derived from CD22. Representative nucleotide sequences such constructs are shown in SEQ ID NOs: 152-153, 160, 164 and 167. Representative amino acid sequences for such constructs are shown in SEQ ID NOs: 170-171, 178, 182 and 185.

In one embodiment, the disclosure provides a BCD construct comprising an association domain derived from CD19 and an inhibitory domain derived from CD22. Representative nucleotide sequences such constructs are shown in SEQ ID NOs: 154, 156, 161, 162 and 165. Representative amino acid sequences for such constructs are shown in SEQ ID NOs: 172, 174, 179, 180 and 183.

In one embodiment, the disclosure provides a BCD construct comprising an association domain derived from CD19 and an inhibitory domain derived from SHP1. A representative nucleotide sequence such a construct is shown in SEQ ID NOs: 155. A representative amino acid sequence for such a construct is shown in SEQ ID NO: 173.

In one embodiment, the disclosure provides a BCD construct comprising an association domain derived from CD64 and an inhibitory domain derived from CD32b. Representative nucleotide sequences such constructs are shown in SEQ ID NOs: 157 and 158. Representative amino acid sequences for such constructs are shown in SEQ ID NOs: 175 and 176.

In one embodiment, the disclosure provides a BCD construct comprising an association domain derived from Syk and an inhibitory domain derived from SHP1. Representative nucleotide sequences such constructs are shown in SEQ ID NOs: 232-234. Representative amino acid sequences for such constructs are shown in SEQ ID NOs: 238-240.

In one embodiment, the disclosure provides a BCD construct comprising an association domain derived from CD19, CD79a or CD79b and an inhibitory domain derived from Csk (e.g., a constitutively active Csk) Representative nucleotide sequences such constructs are shown in SEQ ID NOs: 235-237. Representative amino acid sequences for such constructs are shown in SEQ ID NOs: 241-243.

NK Cell Disruptor Constructs

In one embodiment, an immune cell disruptor polynucleotide of the disclosure is an NK cell disruptor (NKCD) construct that inhibits the activity of an NK cell when expressed intracellularly in the NK cell. Inhibiting NK cell activity can result in, for example, decreased NK cell proliferation, decreased NK cell cytokine production and/or decreased NK cell cytolytic activity.

An NKCD polynucleotide construct encodes a chimeric polypeptide that associates with at least one component of an NK cell and disrupts normal signal transduction activity in the NK cell. By interfering with (i.e., disrupting, altering, inhibiting) the normal signal transduction activity in the NK cell, a NKCD polypeptide can increase the NK cell activation threshold such that greater stimulation is necessary for the NK cell to respond, thereby resulting in inhibition of NK cell activity in the presence of the NKCD as compared to the level of activity in the absence of the NKCD.

An NKCD polypeptide is a chimeric polypeptide comprising at least two portions (i.e., domains or motifs), a first portion that mediates association of the chimeric polypeptide with at least one membrane or signaling complex component of the NK cell (the “association domain” or AD) and a second portion that mediates the inhibitory effect of the NKCD, through disrupting normal signal transduction in the NK cell (the “inhibitory domain” or ID).

The association domain of an NKCD can be derived from any of a number of different types of NK cell components that interact with other components within the NK cell, including membrane receptor-associated components, membrane receptor components, transmembrane-associated components or intracellular-associated components. The inhibitory domain of the NKCD can be derived from any of a number of different types of NK cell components that are involved in regulating signaling pathway activity in the NK cells, including phosphatases, inhibitory kinases and ITIM-containing proteins.

NK cell activation is controlled by a dynamic balance between complementary and antagonistic pathways that are initiated upon interaction with potential target cells. NK cells express an array of activating cell surface receptors that can trigger cytolytic programs, as well as cytokine or chemokine secretion, such as 2B4. Some of these activating cell surface receptors initiate protein tyrosine kinase (PTK)-dependent pathways through noncovalent associations with transmembrane signaling adaptors that harbor intracytoplasmic ITAMs (immunoreceptor tyrosine-based activation motifs). Additional cell surface receptors that are not directly coupled to ITAMs also participate in NK cell activation. These include NKG2D, which is noncovalently associated to the DAP10 transmembrane signaling adaptor, as well as integrins and cytokine receptors. NK cells also express cell surface inhibitory receptors that antagonize activating pathways through protein tyrosine phosphatases (PTPs). These inhibitory cell surface receptors are characterized by intracytoplasmic ITIMs (immunoreceptor tyrosine-based inhibition motifs).

NK proteins involved in activation of signaling pathways from which an association domain for an NKCD can be derived include 2B4, NKG2D, DAP10, Src family kinases (including Lck, Fyn, Src, Lyn, Yes and Fgr), PLCγ2 and Vay.

NK proteins involved in inhibition of signaling pathways from which an inhibitory domain for an NKCD can be derived include CD158, CD94-NKG2A, LILR, SHP1 SHP2 and LAIR1.

Dendritic Cell Disruptor Constructs

In one embodiment, an immune cell disruptor polynucleotide of the disclosure is a dendritic cell disruptor (DCD) construct that inhibits the activity of a dendritic cell when expressed intracellularly in the dendritic cell. Inhibiting dendritic cell activity can result in, for example, decreased dendritic cell proliferation, decreased dendritic cell cytokine production and/or decreased dendritic cell effector function (e.g., antigen presentation).

A DCD polynucleotide construct encodes a chimeric polypeptide that associates with at least one component of a DC and disrupts normal signal transduction activity in the DC. By interfering with (i.e., disrupting, altering, inhibiting) the normal signal transduction activity in the DC, a DCD polypeptide can increase the DC activation threshold such that greater stimulation is necessary for the DC to respond, thereby resulting in inhibition of DC activity in the presence of the DCD as compared to the level of activity in the absence of the DCD.

A DCD polypeptide is a chimeric polypeptide comprising at least two portions (i.e., domains or motifs), a first portion that mediates association of the chimeric polypeptide with at least one membrane or signaling complex component of the dendritic cell (the “association domain” or AD) and a second portion that mediates the inhibitory effect of the DCD, through disrupting normal signal transduction in the dendritic cell (the “inhibitory domain” or ID).

The association domain of a DCD can be derived from any of a number of different types of DC components that interact with other components within the DC, including membrane receptor-associated components, membrane receptor components, transmembrane-associated components or intracellular-associated components. The inhibitory domain of the DCD can be derived from any of a number of different types of DC components that are involved in regulating signaling pathway activity in the DC, including phosphatases, inhibitory kinases and ITIM-containing proteins.

DCs detect pathogens via pattern recognition receptors (PRRs), which recognize various molecular structures referred to as pathogen-associated molecular patterns (PAMPs), e.g. lipopolysaccharides, lipoteichoic acids, flagellin and nucleic acids. Membrane-associated PRRs, like the Toll-like receptors (TLRs) and C-type lectin receptors (CLRs) respond to extracellular pathogens, while cytosolic PRRs, including RIG-I-like receptors (RLRs) and NOD-like receptors (NLRs) sense intracellular pathogens. These receptors also interact with intracellular adaptor proteins and stimulate activation of activatory kinases. DC activation is inhibited by various negative regulators of signaling activity.

DC proteins involved in activation of signaling pathways from which an association domain for a DCD can be derived include TLR3, TLR4, RIG-1, MDA-5, adaptor proteins MyD88, TRIF, TRAM and TIRAP, and JAK and STAT molecules involved in the JAK/STAT signaling pathway.

DC proteins involved in inhibition of signaling pathways from which an inhibitory domain for a DCD can be derived include A20, SIKE, PINI, RNF125, NLRX1 and SOCS1.

Macrophage Cell Disruptor Constructs

In one embodiment, an immune cell disruptor polynucleotide of the disclosure is a macrophage disruptor (MPD) construct that inhibits the activity of a macrophage when expressed intracellularly in the macrophage. Inhibiting macrophage activity can result in, for example, decreased macrophage proliferation, decreased macrophage cytokine production and/or decreased macrophage effector function (e.g., antigen presentation).

An MPD polynucleotide construct encodes a chimeric polypeptide that associates with at least one component of a macrophage and disrupts normal signal transduction activity in the macrophage. By interfering with (i.e., disrupting, altering, inhibiting) the normal signal transduction activity in the macrophage, a MPD polypeptide can increase the macrophage activation threshold such that greater stimulation is necessary for the macrophage to respond, thereby resulting in inhibition of macrophage activity in the presence of the MPD as compared to the level of activity in the absence of the MPD.

An MPD polypeptide is a chimeric polypeptide comprising at least two portions (i.e., domains or motifs), a first portion that mediates association of the chimeric polypeptide with at least one membrane or signaling complex component of the macrophage (the “association domain” or AD) and a second portion that mediates the inhibitory effect of the MPD, through disrupting normal signal transduction in the macrophage (the “inhibitory domain” or ID).

The association domain of a MPD can be derived from any of a number of different types of macrophage components that interact with other components within the macrophage, including membrane receptor-associated components, membrane receptor components, transmembrane-associated components or intracellular-associated components. The inhibitory domain of the MPD can be derived from any of a number of different types of macrophage components that are involved in regulating signaling pathway activity in the macrophage, including phosphatases, inhibitory kinases and ITIM-containing proteins.

Classical activation of macrophages typically involves Toll-like receptors (TLRs) and TLR ligands acting in a MyD88-dependent manner. In addition to MyD88, some TLR ligands can also activate TIR-domain-containing adaptor protein inducing IFNβ (TRIF)-dependent pathways, which signal through IFN-regulatory factor 3 (IRF3). Gene activation is inducted by a combination of transcription factors, including signal transducer and activator of transcription (STAT) molecules, which are activated following IFNγ receptor ligation, and nuclear factor-κB (NFκB) and mitogen-activated protein kinases (MAPKs), which are activated in response to TLR or TNF receptor ligation. Downregulation of macrophage activation is mediated by phosphatases including SHP1 and PTP-1B.

Macrophage proteins involved in activation of signaling pathways from which an association domain for a MPD can be derived include TLRs, MyD88, TRIF, IRF3, STATs, JAKs, MAPK and ERKs.

Macrophage proteins involved in inhibition of signaling pathways from which an inhibitory domain for a MPD can be derived include SHP-1 and PTP-1B.

Messenger RNA (mRNA)

In some embodiments, the disclosure provides an mRNA for use in the constructs, formulations and methods described herein. An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An exemplary 5′ UTR for use in the constructs is shown in SEQ ID NO: 186. An exemplary 3′ UTR for use in the constructs is shown in SEQ ID NO: 187. Exemplary 3′ UTR comprising miR binding sites for use in the constructs are shown in SEQ ID NOs: 212-221. In one embodiment, hepatocyte expression is reduced by including miR122 binding sites. An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,03′GpppG, m27,03′GppppG, m27,02′GppppG, m7Gpppm7G, m73′dGpppG, m27,03′GpppG, m27,03′GppppG, and m27,02′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In one embodiment, the polynucleotides of the present disclosure may include a sequence encoding a self-cleaving peptide. The self-cleaving peptide may be, but is not limited to, a 2A peptide. A variety of 2A peptides are known and available in the art and may be used, including e.g., the foot and mouth disease virus (FMDV) 2A peptide, the equine rhinitis A virus 2A peptide, the Thosea asigna virus 2A peptide, and the porcine teschovirus-1 2A peptide. 2A peptides are used by several viruses to generate two proteins from one transcript by ribosome-skipping, such that a normal peptide bond is impaired at the 2A peptide sequence, resulting in two discontinuous proteins being produced from one translation event. As a non-limiting example, the 2A peptide may have the protein sequence: GSGATNFSLLKQAGDVEENPGP (SEQ ID NO: 226), fragments or variants thereof. In one embodiment, the 2A peptide cleaves between the last glycine and last proline. As another non-limiting example, the polynucleotides of the present disclosure may include a polynucleotide sequence encoding the 2A peptide having the protein sequence GSGATNFSLLKQAGDVEENPGP (SEQ ID NO: 226) fragments or variants thereof. One example of a polynucleotide sequence encoding the 2A peptide is: GGAAGCGGAGCTACTAACTTCAGCCTGCTGAAGCAGGCTGGAGACGTGGAGGAGAA CCCTGGACCT (SEQ ID NO: 227). In one illustrative embodiment, a 2A peptide is encoded by the following sequence: 5′-TCCGGACTCAGATCCGGGGATCTCAAAATTGTCGCTCCTGTCAAACAAACTCTTAAC TTTGATTTACTCAAACTGGCTGGGGATGTAGAAAGCAATCCAGGTCCACTC-3′(SEQ ID NO: 228). The polynucleotide sequence of the 2A peptide may be modified or codon optimized by the methods described herein and/or are known in the art.

In one embodiment, this sequence may be used to separate the coding regions of two or more polypeptides of interest. As a non-limiting example, the sequence encoding the F2A peptide may be between a first coding region A and a second coding region B (A-F2Apep-B). The presence of the F2A peptide results in the cleavage of the one long protein between the glycine and the proline at the end of the F2A peptide sequence (NPGP is cleaved to result in NPG and P) thus creating separate protein A (with 21 amino acids of the F2A peptide attached, ending with NPG) and separate protein B (with 1 amino acid, P, of the F2A peptide attached). Likewise, for other 2A peptides (P2A, T2A and E2A), the presence of the peptide in a long protein results in cleavage between the glycine and proline at the end of the 2A peptide sequence (NPGP is cleaved to result in NPG and P). Protein A and protein B may be the same or different peptides or polypeptides of interest. In particular embodiments, protein A is a polypeptide that induces immunogenic cell death and protein B is another polypeptide that stimulates an inflammatory and/or immune response and/or regulates immune responsiveness (as described further below).

Untranslated Regions (UTRs)

Translation of a polynucleotide comprising an open reading frame encoding a polypeptide can be controlled and regulated by a variety of mechanisms that are provided by various cis-acting nucleic acid structures. For example, naturally-occurring, cis-acting RNA elements that form hairpins or other higher-order (e.g., pseudoknot) intramolecular mRNA secondary structures can provide a translational regulatory activity to a polynucleotide, wherein the RNA element influences or modulates the initiation of polynucleotide translation, particularly when the RNA element is positioned in the 5′ UTR close to the 5′-cap structure (Pelletier and Sonenberg (1985) Cell 40(3):515-526; Kozak (1986) Proc Natl Acad Sci 83:2850-2854). Untranslated regions (UTRs) are nucleic acid sections of a polynucleotide before a start codon (5′ UTR) and after a stop codon (3′ UTR) that are not translated. In some embodiments, a polynucleotide (e.g., a ribonucleic acid (RNA), e.g., a messenger RNA (mRNA)) of the disclosure comprising an open reading frame (ORF) encoding an ARG1 polypeptide further comprises UTR (e.g., a 5′ UTR or functional fragment thereof, a 3′ UTR or functional fragment thereof, or a combination thereof).

Cis-acting RNA elements can also affect translation elongation, being involved in numerous frameshifting events (Namy et al., (2004) Mol Cell 13(2):157-168). Internal ribosome entry sequences (IRES) represent another type of cis-acting RNA element that are typically located in 5′ UTRs, but have also been reported to be found within the coding region of naturally-occurring mRNAs (Holcik et al. (2000) Trends Genet 16(10):469-473). In cellular mRNAs, IRES often coexist with the 5′-cap structure and provide mRNAs with the functional capacity to be translated under conditions in which cap-dependent translation is compromised (Gebauer et al., (2012) Cold Spring Harb Perspect Biol 4(7):a012245). Another type of naturally-occurring cis-acting RNA element comprises upstream open reading frames (uORFs). Naturally-occurring uORFs occur singularly or multiply within the 5′ UTRs of numerous mRNAs and influence the translation of the downstream major ORF, usually negatively (with the notable exception of GCN4 mRNA in yeast and ATF4 mRNA in mammals, where uORFs serve to promote the translation of the downstream major ORF under conditions of increased eIF2 phosphorylation (Hinnebusch (2005) Annu Rev Microbiol 59:407-450)). Additional exemplary translational regulatory activities provided by components, structures, elements, motifs, and/or specific sequences comprising polynucleotides (e.g., mRNA) include, but are not limited to, mRNA stabilization or destabilization (Baker & Parker (2004) Curr Opin Cell Biol 16(3):293-299), translational activation (Villalba et al., (2011) Curr Opin Genet Dev 21(4):452-457), and translational repression (Blumer et al., (2002) Mech Dev 110(1-2):97-112). Studies have shown that naturally-occurring, cis-acting RNA elements can confer their respective functions when used to modify, by incorporation into, heterologous polynucleotides (Goldberg-Cohen et al., (2002) J Biol Chem 277(16):13635-13640).

Modified mRNAs Comprising Functional RNA Elements

The present disclosure provides synthetic polynucleotides comprising a modification (e.g., an RNA element), wherein the modification provides a desired translational regulatory activity. In some embodiments, the disclosure provides a polynucleotide comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In some embodiments, the desired translational regulatory activity is a cis-acting regulatory activity. In some embodiments, the desired translational regulatory activity is an increase in the residence time of the 43S pre-initiation complex (PIC) or ribosome at, or proximal to, the initiation codon. In some embodiments, the desired translational regulatory activity is an increase in the initiation of polypeptide synthesis at or from the initiation codon. In some embodiments, the desired translational regulatory activity is an increase in the amount of polypeptide translated from the full open reading frame. In some embodiments, the desired translational regulatory activity is an increase in the fidelity of initiation codon decoding by the PIC or ribosome. In some embodiments, the desired translational regulatory activity is inhibition or reduction of leaky scanning by the PIC or ribosome. In some embodiments, the desired translational regulatory activity is a decrease in the rate of decoding the initiation codon by the PIC or ribosome. In some embodiments, the desired translational regulatory activity is inhibition or reduction in the initiation of polypeptide synthesis at any codon within the mRNA other than the initiation codon. In some embodiments, the desired translational regulatory activity is inhibition or reduction of the amount of polypeptide translated from any open reading frame within the mRNA other than the full open reading frame. In some embodiments, the desired translational regulatory activity is inhibition or reduction in the production of aberrant translation products. In some embodiments, the desired translational regulatory activity is a combination of one or more of the foregoing translational regulatory activities.

Accordingly, the present disclosure provides a polynucleotide, e.g., an mRNA, comprising an RNA element that comprises a sequence and/or an RNA secondary structure(s) that provides a desired translational regulatory activity as described herein. In some aspects, the mRNA comprises an RNA element that comprises a sequence and/or an RNA secondary structure(s) that promotes and/or enhances the translational fidelity of mRNA translation. In some aspects, the mRNA comprises an RNA element that comprises a sequence and/or an RNA secondary structure(s) that provides a desired translational regulatory activity, such as inhibiting and/or reducing leaky scanning. In some aspects, the disclosure provides an mRNA that comprises an RNA element that comprises a sequence and/or an RNA secondary structure(s) that inhibits and/or reduces leaky scanning thereby promoting the translational fidelity of the mRNA. In some embodiments, the RNA element comprises natural and/or modified nucleotides. In some embodiments, the RNA element comprises of a sequence of linked nucleotides, or derivatives or analogs thereof, that provides a desired translational regulatory activity as described herein. In some embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

In some aspects, the disclosure provides an mRNA having one or more structural modifications that inhibits leaky scanning and/or promotes the translational fidelity of mRNA translation, wherein at least one of the structural modifications is a GC-rich RNA element. In some aspects, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA. In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is 70-80% cytosine, 60-70% cytosine, 50%-60% cytosine, 40-50% cytosine, 30-40% cytosine bases. In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is about 80% cytosine, about 70% cytosine, about 60% cytosine, about 50% cytosine, about 40% cytosine, or about 30% cytosine.

In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is 70-80% cytosine, 60-70% cytosine, 50%-60% cytosine, 40-50% cytosine, or 30-40% cytosine. In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of 3, 4, 5, 6, 7, 8, 9, 10, 11, 12, 13, 14, 15, 16, 17, 18, 19, or 20 nucleotides, or derivatives or analogs thereof, linked in any order, wherein the sequence composition is >50% cytosine. In some embodiments, the sequence composition is >55% cytosine, >60% cytosine, >65% cytosine, >70% cytosine, >75% cytosine, >80% cytosine, >85% cytosine, or >90% cytosine.

In other aspects, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n, wherein n=1 to 10, n=2 to 8, n=3 to 6, or n=4 to 5. In some embodiments, the sequence comprises a repeating GC-motif [CCG]n, wherein n=1, 2, 3, 4 or 5. In some embodiments, the sequence comprises a repeating GC-motif [CCG]n, wherein n=1, 2, or 3. In some embodiments, the sequence comprises a repeating GC-motif [CCG]n, wherein n=1. In some embodiments, the sequence comprises a repeating GC-motif [CCG]n, wherein n=2. In some embodiments, the sequence comprises a repeating GC-motif [CCG]n, wherein n=3. In some embodiments, the sequence comprises a repeating GC-motif [CCG]n, wherein n=4. In some embodiments, the sequence comprises a repeating GC-motif [CCG]n, wherein n=5.

In another aspect, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element comprises any one of the sequences set forth in Table 1. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In other aspects, the disclosure provides a modified mRNA comprising at least one modification, wherein at least one modification is a GC-rich RNA element comprising the sequence V1 [CCCCGGCGCC (SEQ ID NO:194)] as set forth in Table 1, or derivatives or analogs thereof, preceding a Kozak consensus sequence in the 5′ UTR of the mRNA. In some embodiments, the GC-rich element comprises the sequence V1 as set forth in Table 1 located immediately adjacent to and upstream of the Kozak consensus sequence in the 5′ UTR of the mRNA. In some embodiments, the GC-rich element comprises the sequence V1 as set forth in Table 1 located 1, 2, 3, 4, 5, 6, 7, 8, 9 or 10 bases upstream of the Kozak consensus sequence in the 5′ UTR of the mRNA. In other embodiments, the GC-rich element comprises the sequence V1 as set forth in Table 1 located 1-3, 3-5, 5-7, 7-9, 9-12, or 12-15 bases upstream of the Kozak consensus sequence in the 5′ UTR of the mRNA.

In other aspects, the disclosure provides a modified mRNA comprising at least one modification, wherein at least one modification is a GC-rich RNA element comprising the sequence V2 [CCCCGGC (SEQ ID NO:195)] as set forth in Table 1, or derivatives or analogs thereof, preceding a Kozak consensus sequence in the 5′ UTR of the mRNA. In some embodiments, the GC-rich element comprises the sequence V2 as set forth in Table 1 located immediately adjacent to and upstream of the Kozak consensus sequence in the 5′ UTR of the mRNA. In some embodiments, the GC-rich element comprises the sequence V2 as set forth in Table 1 located 1, 2, 3, 4, 5, 6, 7, 8, 9 or 10 bases upstream of the Kozak consensus sequence in the 5′ UTR of the mRNA. In other embodiments, the GC-rich element comprises the sequence V2 as set forth in Table 1 located 1-3, 3-5, 5-7, 7-9, 9-12, or 12-15 bases upstream of the Kozak consensus sequence in the 5′ UTR of the mRNA.

In other aspects, the disclosure provides a modified mRNA comprising at least one modification, wherein at least one modification is a GC-rich RNA element comprising the sequence EK [GCCGCC (SEQ ID NO:193)] as set forth in Table 1, or derivatives or analogs thereof, preceding a Kozak consensus sequence in the 5′ UTR of the mRNA. In some embodiments, the GC-rich element comprises the sequence EK as set forth in Table 1 located immediately adjacent to and upstream of the Kozak consensus sequence in the 5′ UTR of the mRNA. In some embodiments, the GC-rich element comprises the sequence EK as set forth in Table 1 located 1, 2, 3, 4, 5, 6, 7, 8, 9 or 10 bases upstream of the Kozak consensus sequence in the 5′ UTR of the mRNA. In other embodiments, the GC-rich element comprises the sequence EK as set forth in Table 1 located 1-3, 3-5, 5-7, 7-9, 9-12, or 12-15 bases upstream of the Kozak consensus sequence in the 5′ UTR of the mRNA.

In yet other aspects, the disclosure provides a modified mRNA comprising at least one modification, wherein at least one modification is a GC-rich RNA element comprising the sequence V1 [CCCCGGCGCC (SEQ ID NO:194)] as set forth in Table 1, or derivatives or analogs thereof, preceding a Kozak consensus sequence in the 5′ UTR of the mRNA, wherein the 5′ UTR comprises the following sequence shown in Table 1:

(SEQ ID NO: 189)
GGGAAAUAAGAGAGAAAAGAAGAGUAAGAAGAAAUAUAAGA.

The skilled artisan will of course recognize that all Us in the RNA sequences described herein will be Ts in a corresponding template DNA sequence, for example, in DNA templates or constructs from which mRNAs of the disclosure are transcribed, e.g., via IVT.

In some embodiments, the GC-rich element comprises the sequence V1 as set forth in Table 1 located immediately adjacent to and upstream of the Kozak consensus sequence in the 5′ UTR sequence shown in Table 1. In some embodiments, the GC-rich element comprises the sequence V1 as set forth in Table 1 located 1, 2, 3, 4, 5, 6, 7, 8, 9 or 10 bases upstream of the Kozak consensus sequence in the 5′ UTR of the mRNA, wherein the 5′ UTR comprises the following sequence shown in Table 1:

(SEQ ID NO: 189)
GGGAAAUAAGAGAGAAAAGAAGAGUAAGAAGAAAUAUAAGA.

In other embodiments, the GC-rich element comprises the sequence V1 as set forth in Table 1 located 1-3, 3-5, 5-7, 7-9, 9-12, or 12-15 bases upstream of the Kozak consensus sequence in the 5′ UTR of the mRNA, wherein the 5′ UTR comprises the following sequence shown in Table 1:

(SEQ ID NO: 189)
GGGAAAUAAGAGAGAAAAGAAGAGUAAGAAGAAAUAUAAGA.

In some embodiments, the 5′ UTR comprises the following sequence set forth in Table 1:

(SEQ ID NO: 186)
GGGAAAUAAGAGAGAAAAGAAGAGUAAGAAGAAAUAUAAGACCCCGGCG
CCGCCACC

TABLE 1

| 5′ UTRs | 5′ UTR Sequence |
|---|---|
| Standard | GGGAAAUAAGAGAGAAAAGAAGAGUAAGAAGAA |
| | AUAUAAGA (SEQ: 189) |
| Standard | GGGAAAUAAGAGAGAAAAGAAGAGUAAGAAGAA |
| | AUAUAAGAGCCACC (SEQ ID NO: 190) |
| V1-UTR | GGGAAAUAAGAGAGAAAAGAAGAGUAAGAAGAA |
| | AUAUAAGACCCCGGCGCCGCCACC (SEQ ID |
| | NO: 186) |
| V2-UTR | GGGAAAUAAGAGAGAAAAGAAGAGUAAGAAGAA |
| | AUAUAAGACCCCGGCGCCACC (SEQ ID NO: 191) |
| GC-Rich RNA Elements | Sequence |
| K0 (Traditional Kozak consensus) | [GCCA/GCC] (SEQ ID NO: 192) |
| EK | [GCCGCC] (SEQ ID NO: 193) |
| V1 | [CCCCGGCGCC] (SEQ ID NO: 194) |
| V2 | [CCCCGGC] (SEQ ID NO: 195) |
| (CCG)ₙ, where n = 1 − 10 | [CCG]ₙ |
| (GCC)ₙ, where n = 1 − 10 | [GCC]ₙ |

In another aspect, the disclosure provides a modified mRNA comprising at least one modification, wherein at least one modification is a GC-rich RNA element comprising a stable RNA secondary structure comprising a sequence of nucleotides, or derivatives or analogs thereof, linked in an order which forms a hairpin or a stem-loop. In one embodiment, the stable RNA secondary structure is upstream of the Kozak consensus sequence. In another embodiment, the stable RNA secondary structure is located about 30, about 25, about 20, about 15, about 10, or about 5 nucleotides upstream of the Kozak consensus sequence. In another embodiment, the stable RNA secondary structure is located about 20, about 15, about 10 or about 5 nucleotides upstream of the Kozak consensus sequence. In another embodiment, the stable RNA secondary structure is located about 5, about 4, about 3, about 2, about 1 nucleotides upstream of the Kozak consensus sequence. In another embodiment, the stable RNA secondary structure is located about 15-30, about 15-20, about 15-25, about 10-15, or about 5-10 nucleotides upstream of the Kozak consensus sequence. In another embodiment, the stable RNA secondary structure is located 12-15 nucleotides upstream of the Kozak consensus sequence. In another embodiment, the stable RNA secondary structure has a deltaG of about −30 kcal/mol, about −20 to −30 kcal/mol, about −20 kcal/mol, about −10 to −20 kcal/mol, about −10 kcal/mol, about −5 to ˜10 kcal/mol.

In another embodiment, the modification is operably linked to an open reading frame encoding a polypeptide and wherein the modification and the open reading frame are heterologous.

In another embodiment, the sequence of the GC-rich RNA element is comprised exclusively of guanine (G) and cytosine (C) nucleobases.

RNA elements that provide a desired translational regulatory activity as described herein can be identified and characterized using known techniques, such as ribosome profiling. Ribosome profiling is a technique that allows the determination of the positions of PICs and/or ribosomes bound to mRNAs (see e.g., Ingolia et al., (2009) Science 324(5924):218-23, incorporated herein by reference). The technique is based on protecting a region or segment of mRNA, by the PIC and/or ribosome, from nuclease digestion. Protection results in the generation of a 30-bp fragment of RNA termed a ‘footprint’. The sequence and frequency of RNA footprints can be analyzed by methods known in the art (e.g., RNA-seq). The footprint is roughly centered on the A-site of the ribosome. If the PIC or ribosome dwells at a particular position or location along an mRNA, footprints generated at these position would be relatively common. Studies have shown that more footprints are generated at positions where the PIC and/or ribosome exhibits decreased processivity and fewer footprints where the PIC and/or ribosome exhibits increased processivity (Gardin et al., (2014) eLife 3:e03735). In some embodiments, residence time or the time of occupancy of the PIC or ribosome at a discrete position or location along a polynucleotide comprising any one or more of the RNA elements described herein is determined by ribosome profiling.

A UTR can be homologous or heterologous to the coding region in a polynucleotide. In some embodiments, the UTR is homologous to the ORF encoding the ARG1 polypeptide. In some embodiments, the UTR is heterologous to the ORF encoding the ARG1 polypeptide. In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively. Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO:196), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR. Co-owned International Patent Application No. PCT/US2014/021522 (Publ. No. WO/2014/164253, incorporated herein by reference in its entirety) provides a listing of exemplary UTRs that can be utilized in the polynucleotide of the present disclosure as flanking regions to an ORF.

Exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (ColIA2), collagen type I, alpha 1 (ColIA1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1). In some embodiments, the 5′ UTR is selected from the group consisting of a β-globin 5′ UTR; a 5′UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 al (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the disclosure. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs. In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the disclosure comprise a 5′ UTR and/or a 3′ UTR selected from any of the UTRs disclosed herein. In some embodiments, the 5′ UTR comprises:

5′ UTR-001 (Upstream UTR)
(SEQ ID NO: 190)
(GGGAAAUAAGAGAGAAAAGAAGAGUAAGAAGAAAUAUAAGAGCCACC);
5′ UTR-002 (Upstream UTR)
(SEQ ID NO: 197)
(GGGAGAUCAGAGAGAAAAGAAGAGUAAGAAGAAAUAUAAGAGCCACC);
5′ UTR-003 (Upstream UTR) (See W02016/100812);
5′ UTR-004 (Upstream UTR)
(SEQ ID NO: 198)
(GGGAGACAAGCUUGGCAUUCCGGUACUGUUGGUAAAGCCACC);
5′ UTR-005 (Upstream UTR)
(SEQ ID NO: 199)
(GGGAGAUCAGAGAGAAAAGAAGAGUAAGAAGAAAUAUAAGAGCCACC);
5′ UTR-006 (Upstream UTR) (See W02016/100812);
5′ UTR-007 (Upstream UTR)
(SEQ ID NO: 200)
(GGGAGACAAGCUUGGCAUUCCGGUACUGUUGGUAAAGCCACC);
5′ UTR-008 (Upstream UTR)
(SEQ ID NO: 201)
(GGGAAUUAACAGAGAAAAGAAGAGUAAGAAGAAAUAUAAGAGCCACC);
5′ UTR-009 (Upstream UTR)
(SEQ ID NO: 202)
(GGGAAAUUAGACAGAAAAGAAGAGUAAGAAGAAAUAUAAGAGCCACC);
5′ UTR-010, Upstream
(SEQ ID NO: 203)
(GGGAAAUAAGAGAGUAAAGAACAGUAAGAAGAAAUAUAAGAGCCACC);
5′ UTR-011 (Upstream UTR)
(SEQ ID NO: 204)
(GGGAAAAAAGAGAGAAAAGAAGACUAAGAAGAAAUAUAAGAGCCACC);
5′ UTR-012 (Upstream UTR)
(SEQ ID NO: 205)
(GGGAAAUAAGAGAGAAAAGAAGAGUAAGAAGAUAUAUAAGAGCCACC);
5′ UTR-013 (Upstream UTR)
(SEQ ID NO: 206)
(GGGAAAUAAGAGACAAAACAAGAGUAAGAAGAAAUAUAAGAGCCACC);
5′ UTR-014 (Upstream UTR)
(SEQ ID NO: 207)
(GGGAAAUUAGAGAGUAAAGAACAGUAAGUAGAAUUAAAAGAGCCACC);
5′ UTR-015 (Upstream UTR)
(SEQ ID NO: 208)
(GGGAAAUAAGAGAGAAUAGAAGAGUAAGAAGAAAUAUAAGAGCCACC);
5′ UTR-016 (Upstream UTR)
(SEQ ID NO: 209)
(GGGAAAUAAGAGAGAAAAGAAGAGUAAGAAGAAAAUUAAGAGCCACC);
5′ UTR-017 (Upstream UTR);
(SEQ ID NO: 210)
(GGGAAAUAAGAGAGAAAAGAAGAGUAAGAAGAAAUUUAAGAGCCACC);
or
5′ UTR-018 (Upstream UTR) 5′ UTR
(SEQ ID NO: 211)
(UCAAGCUUUUGGACCCUCGUACAGAAGCUAAUACGACUCACUAUAGGGA
AAUAAGAGAGAAAAGAAGAGUAAGAAGAAAUAUAAGAGCCACC).

In some embodiments, the 3′ UTR comprises:

142-3p 3′ UTR
(UTR including miR142-3p binding site)
(SEQ ID NO: 212)
(UGAUAAUAGUCCAUAAAGUAGGAAACACUACAGCUGGAGCCUCGGUGGC
CAUGCUUCUUGCCCCUUGGGCCUCCCCCCAGCCCCUCCUCCCCUUCCUGC
ACCCGUACCCCCGUGGUCUUUGAAUAAAGUCUGAGUGGGCGGC);
142-3p 3′ UTR
(UTR including miR142-3p binding site)
(SEQ ID NO: 213)
(UGAUAAUAGGCUGGAGCCUCGGUGGCUCCAUAAAGUAGGAAACACUACA
CAUGCUUCUUGCCCCUUGGGCCUCCCCCCAGCCCCUCCUCCCCUUCCUGC
ACCCGUACCCCCGUGGUCUUUGAAUAAAGUCUGAGUGGGCGGC);
or
142-3p 3′ UTR
(UTR including miR142-3p binding site)
(SEQ ID NO: 214)
(UGAUAAUAGGCUGGAGCCUCGGUGGCCAUGCUUCUUGCCCCUUCCAUAA
AGUAGGAAACACUACAUGGGCCUCCCCCCAGCCCCUCCUCCCCUUCCUGC
ACCCGUACCCCCGUGGUCUUUGAAUAAAGUCUGAGUGGGCGGC);
142-3p 3′ UTR
(UTR including miR142-3p binding site)
(SEQ ID NO: 215)
(UGAUAAUAGGCUGGAGCCUCGGUGGCCAUGCUUCUUGCCCCUUGGGCCU
CCCCCCAGUCCAUAAAGUAGGAAACACUACACCCCUCCUCCCCUUCCUGC
ACCCGUACCCCCGUGGUCUUUGAAUAAAGUCUGAGUGGGCGGC);
142-3p 3′ UTR
(UTR including miR142-3p binding site)
(SEQ ID NO: 216)
(UGAUAAUAGGCUGGAGCCUCGGUGGCCAUGCUUCUUGCCCCUUGGGCCU
CCCCCCAGCCCCUCCUCCCCUUCUCCAUAAAGUAGGAAACACUACACUGC
ACCCGUACCCCCGUGGUCUUUGAAUAAAGUCUGAGUGGGCGGC);
142-3p 3′ UTR
(UTR including miR142-3p binding site)
(SEQ ID NO: 217)
(UGAUAAUAGGCUGGAGCCUCGGUGGCCAUGCUUCUUGCCCCUUGGGCCU
CCCCCCAGCCCCUCCUCCCCUUCCUGCACCCGUACCCCCUCCAUAAAGUA
GGAAACACUACAGUGGUCUUUGAAUAAAGUCUGAGUGGGCGGC).
142-3p 3′ UTR
(UTR including miR142-3p binding site)
(SEQ ID NO: 218)
(UGAUAAUAGGCUGGAGCCUCGGUGGCCAUGCUUCUUGCCCCUUGGGCCU
CCCCCCAGCCCCUCCUCCCCUUCCUGCACCCGUACCCCCGUGGUCUUUGA
AUAAAGUUCCAUAAAGUAGGAAACACUACACUGAGUGGGCGGC);
3′ UTR
(miR142 and miR126 binding sites variant 1)
(SEQ ID NO: 219)
(UGAUAAUAGUCCAUAAAGUAGGAAACACUACAGCUGGAGCCUCGGUGGC
CAUGCUUCUUGCCCCUUGGGCCUCCCCCCAGCCCCUCCUCCCCUUCCUGC
ACCCGUACCCCCCGCAUUAUUACUCACGGUACGAGUGGUCUUUGAAUAAA
GUCUGAGUGGGCGGC)
3′ UTR
(miR142 and miR126 binding sites variant 2)
(SEQ ID NO: 220)
(UGAUAAUAGUCCAUAAAGUAGGAAACACUACAGCUGGAGCCUCGGUGGC
CUAGCUUCUUGCCCCUUGGGCCUCCCCCCAGCCCCUCCUCCCCUUCCUGC
ACCCGUACCCCCCGCAUUAUUACUCACGGUACGAGUGGUCUUUGAAUAAA
GUCUGAGUGGGCGGC);
or
3′UTR (miR142-3p binding site variant 3)
(SEQ ID NO: 221)
UGAUAAUAGGCUGGAGCCUCGGUGGCCUAGCUUCUUGCCCCUUGGGCCUC
CCCCCAGCCCCUCCUCCCCUUCCUGCACCCGUACCCCCUCCAUAAAGUAG
GAAACACUACAGUGGUCUUUGAAUAAAGUCUGAGUGGGCGGC.

In certain embodiments, the 5′ UTR and/or 3′ UTR sequence of the disclosure comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of SEQ ID NOs:186, 189-191 and 197-211 and/or 3′ UTR sequences comprises any of SEQ ID NOs:187 and 212-221, and any combination thereof.

In certain embodiments, the 5′ UTR and/or 3′ UTR sequence of the disclosure comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of SEQ ID NOs:186, 189-191 and 197-211 and/or 3′ UTR sequences comprises any of SEQ ID NOs:187 and 212-221, and any combination thereof.

The polynucleotides of the disclosure can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the disclosure. For example, introns or portions of intron sequences can be incorporated into the polynucleotides of the disclosure. Incorporation of intronic sequences can increase protein production as well as polynucleotide expression levels. In some embodiments, the polynucleotide of the disclosure comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation.

5′ Capping

It is desirable to manufacture therapeutic RNAs enzymatically using in vitro transcription (IVT). In general, a DNA-dependent RNA polymerase transcribes a DNA template containing an appropriate promoter into an RNA transcript. The poly(A) tail can be generated co-transcriptionally by incorporating a poly(T) tract in the template DNA or separately by using a poly(A) polymerase. Eukaryotic mRNAs start with a 5′ cap (e.g., a 5′ m7GpppX cap). Typically, the 5′ cap begins with an inverted G with N⁷Me (required for eIF4E binding). A preferred cap, Cap1 contains 2′OMe at the +1 position) followed by any nucleoside at +2 position. This cap can be installed post-transcriptionally, e.g., enzymatically (after transcription) or co-transcriptionally (during transcription).

Post-transcriptional capping can be carried out using the vaccinia capping enzyme and allows for complete capping of the RNA, generating a cap 0 structure on RNA carrying a 5′ terminal triphosphate or diphosphate group, the cap 0 structure being required for efficient translation of the mRNA in vivo. The cap 0 structure can then be further modified into cap 1 using a cap-specific 2′O methyltransferase. Vaccinia capping enzyme and 2′O methyltransferase have been used to generate cap 0 and cap 1 structures on in vitro transcripts, for example, for use in transfecting eukaryotic cells or in mRNA therapeutic applications to drive protein synthesis. While post-transcriptional capping by vaccinia capping enzymes can yield either Cap 0 or Cap 1 structures, it is an expensive process when utilized for large-scale mRNA production, for example, vaccinia is costly and in limited supply and there can be difficulties in purifying an IVT mRNA (e.g., removing S-adenosylmethionine (SAM) and 2′O-methyltransferase). Moreover, capping can be incomplete due to inaccessibility of structured 5′ ends.

Co-transcriptional capping using a cap analog has certain advantages over vaccinia capping, for example, the process requires a simpler workflow (e.g., no need for a purification step between transcription and capping). Traditional co-transcriptional capping methods utilize the dinucleotide ARCA (anti-reverse cap analog) and yield Cap 0 structures. ARCA capping has drawbacks, however, for example, the resulting Cap 0 structures can be immunogenic and the process often results in low yields and/or poorly capped material. Another potential drawback of this approach is a theoretical capping efficiency of <100%, due to competition from the GTP for the starting nucleotide. For example, co-transcriptonal capping using ARCA typically requires a 10:1 ratio of ARCA:GTP to achieve >90% capping (needed to outcompete GTP for initiation).

In some embodiments, mRNAs of the disclosure are comprised of trinucleotide mRNA cap analogs, prepared using co-transcriptional capping methods (e.g., featuring T7 RNA polymerase) for the in vitro synthesis of mRNA. Use of a trinucleotide cap analog may provide a solution to several of the above-described problems associated with vaccinia or ARCA capping. In addition, the methods of co-transcriptional capping described provide flexibility in modifying the penultimate nucleobase which may alter binding behavior, or affect the affinity of these caps towards decapping enzymes, or both, thus potentially improving stability of the respective mRNA. An exemplary trinucleotide for use in the herein-described co-transcriptional capping methods is the m7GpppAG (GAG) trinucleotide. Use of this trinucleotide results in the nucleotide at the +1 position being A instead of G. Both +1G and +1A are caps that can be found in naturally-occurring mRNAs.

T7 RNA polymerase prefers to initiate with 5′ GTP. Accordingly, Most conventional mRNA transcripts start with 5′- [symbol] (based on transcription from a T7 promoter sequence such as 5′TAATACGACTCACTATA [symbol] NNNNNNNNN . . . 3′ (SEQ ID NO: 222) (TATA being referred to as the “TATA box”). T7 RNA polymerase typically transcribes DNA downstream of a T7 promoter (5′ TAATACGACTCACTATAG 3′, (SEQ ID NO: 223) referencing the coding strand). T7 polymerase starts transcription at the underlined G in the promoter sequence. The polymerase then transcribes using the opposite strand as a template from 5′->3′. The first base in the transcript will be a G.

The herein-described processes capitalize on the fact that the T7 enzyme has limited initiation activity with the single nucleotide ATP, driving T7 to initiate with the trinucleotide rather than ATP. The process thus generates an mRNA product with >90% functional cap post-transcription. The process is an efficient “one-pot” mRNA production method that includes, for example, the GAG trinucleotide (GpppAG; mGpppAₘG) in equimolar concentration with the NTPs, GTP, ATP, CTP and UTP. The process features an “A-start” DNA template that initiates transcription with 5′ adenosine (A). As defined herein, “A-start” and “G-start” DNA templates are double-stranded DNA having requisite nucleosides in the template strand, such that the coding strand (and corresponding mRNA) begin with A or G, respectively. For example, a G-start DNA template features a template strand having the nucleobases CC complementary to GG immediately downstream of the TATA box in the T7 promoter (referencing the coding strand), and an A-start DNA template features a template strand having the nucleobases TC complementary to the AG immediately downstream of the TATA box in the T7 promoter (referencing the coding strand).

An exemplary T7 promoter sequence featured in an A-start DNA template of the present disclosure is depicted here:

(SEQ ID NO: 224)
5′TAATACGACTCACTATA [symbol] NNNNNNNNNN... 3′
(SEQ ID NO: 225)
3′ATTATGCTGAGTGATAT [symbol] NNNNNNNNNN... 3′

The trinucleotide-based capping methods described herein provide flexibility in dictating the penultimate nucleobase. The trinucleotide capping methods of the present disclosure provide efficient production of capped mRNA, for example, 95-98% capped mRNA with a natural cap 1 structure.

Poly-A Tails

In some embodiments, a polynucleotide comprising an mRNA encoding a polypeptide of the present disclosure further comprises a poly A tail. In further embodiments, terminal groups on the poly-A tail can be incorporated for stabilization. In other embodiments, a poly-A tail comprises des-3′ hydroxyl tails. The useful poly-A tails can also include structural moieties or 2′-Omethyl modifications as taught by Li et al. (2005) Current Biology 15:1501-1507.

In one embodiment, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present disclosure are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

Start Codon Region

In some embodiments, an mRNA of the present disclosure further comprises regions that are analogous to or function like a start codon region.

In some embodiments, the translation of a polynucleotide initiates on a codon which is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG. See Touriol et al. (2003) Biology of the Cell 95:169-178 and Matsuda and Mauro (2010) PLoS ONE 5:11. As a non-limiting example, the translation of a polynucleotide begins on the alternative start codon ACG. As another non-limiting example, polynucleotide translation begins on the alternative start codon CUG. As yet another non-limiting example, the translation of a polynucleotide begins on the alternative start codon GUG.

Nucleotides flanking a codon that initiates translation such as, but not limited to, a start codon or an alternative start codon, are known to affect the translation efficiency, the length and/or the structure of the polynucleotide. See, e.g., Matsuda and Mauro (2010) PLoS ONE 5:11. Masking any of the nucleotides flanking a codon that initiates translation can be used to alter the position of translation initiation, translation efficiency, length and/or structure of a polynucleotide.

In some embodiments, a masking agent is used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs). See, e.g., Matsuda and Mauro (2010) PLoS ONE 5:11, describing masking agents LNA polynucleotides and EJCs.

In another embodiment, a masking agent is used to mask a start codon of a polynucleotide in order to increase the likelihood that translation will initiate on an alternative start codon. In some embodiments, a masking agent is used to mask a first start codon or alternative start codon in order to increase the chance that translation will initiate on a start codon or alternative start codon downstream to the masked start codon or alternative start codon.

In some embodiments, a start codon or alternative start codon is located within a perfect complement for a miR binding site. The perfect complement of a miR binding site can help control the translation, length and/or structure of the polynucleotide similar to a masking agent. As a non-limiting example, the start codon or alternative start codon is located in the middle of a perfect complement for a miR-122 binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide is removed from the polynucleotide sequence in order to have the translation of the polynucleotide begin on a codon which is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed can further comprise at least one masking agent for the downstream start codon and/or alternative start codons in order to control or attempt to control the initiation of translation, the length of the polynucleotide and/or the structure of the polynucleotide.

Stop Codon Region

In some embodiments, mRNA of the present disclosure can further comprise at least one stop codon or at least two stop codons before the 3′ untranslated region (UTR). The stop codon can be selected from UGA, UAA, and UAG. In some embodiments, the polynucleotides of the present disclosure include the stop codon UGA and one additional stop codon. In a further embodiment the addition stop codon can be UAA. In another embodiment, the polynucleotides of the present disclosure include three stop codons, four stop codons, or more.

Adjusted Uracil Content

In some embodiments of the disclosure, an mRNA may have adjusted uracil content. In some embodiments, the uracil content of the open reading frame (ORF) of the polynucleotide encoding a therapeutic polypeptide relative to the theoretical minimum uracil content of a nucleotide sequence encoding the therapeutic polypeptide (% U_{TM}), is between about 100% and about 150. In some embodiments, the uracil content of the ORF is between about 105% and about 145%, about 105% and about 140%, about 110% and about 140%, about 110% and about 145%, about 115% and about 135%, about 105% and about 135%, about 110% and about 135%, about 115% and about 145%, or about 115% and about 140% of the theoretical minimum uracil content in the corresponding wild-type ORF (% U_{TM}). In other embodiments, the uracil content of the ORF is between about 117% and about 134% or between 118% and 132% of the % U_{TM}. In some embodiments, the uracil content of the ORF encoding a polypeptide is about 115%, about 120%, about 125%, about 130%, about 135%, about 140%, about 145%, or about 150% of the % U_{TM}. In this context, the term “uracil” can refer to an alternative uracil and/or naturally occurring uracil.

In some embodiments, the uracil content of the ORF of the polynucleotide relative to the uracil content of the corresponding wild-type ORF (% Uw_{T}) is less than 100%. In some embodiments, the % Uw_{T} of the polynucleotide is less than about 95%, less than about 90%, less than about 85%, less than 80%, less than 79%, less than 78%, less than 77%, less than 76%, less than 75%, less than 74%, or less than 73%. In some embodiments, the % Uw_{T} of the polynucleotide is between 65% and 73%.

In some embodiments, the uracil content in the ORF of the mRNA encoding a is less than about 50%, about 40%, about 30%, or about 20% of the total nucleobase content in the ORF. In some embodiments, the uracil content in the ORF is between about 15% and about 25% of the total nucleobase content in the ORF. In other embodiments, the uracil content in the ORF is between about 20% and about 30% of the total nucleobase content in the ORF. In one embodiment, the uracil content in the ORF of the mRNA encoding a polypeptide is less than about 20% of the total nucleobase content in the open reading frame. In this context, the term “uracil” can refer to an alternative uracil and/or naturally occurring uracil.

In further embodiments, the ORF of the mRNA encoding a polypeptide having adjusted uracil content has increased cytosine (C), guanine (G), or guanine/cytosine (G/C) content (absolute or relative). In some embodiments, the overall increase in C, G, or G/C content (absolute or relative) of the ORF is at least about 2%, at least about 3%, at least about 4%, at least about 5%, at least about 6%, at least about 7%, at least about 10%, at least about 15%, at least about 20%, at least about 40%, at least about 50%, at least about 60%, at least about 70%, at least about 80%, at least about 90%, at least about 95%, or at least about 100% relative to the G/C content (absolute or relative) of the wild-type ORF. In some embodiments, the G, the C, or the G/C content in the ORF is less than about 100%, less than about 90%, less than about 85%, or less than about 80% of the theoretical maximum G, C, or G/C content of the nucleotide sequence encoding the PBDG polypeptide (% G_{TMX}; % C_{TMX}, or % G/C_{TMX}). In other embodiments, the G, the C, or the G/C content in the ORF is between about 70% and about 80%, between about 71% and about 79%, between about 71% and about 78%, or between about 71% and about 77% of the % G_{TMX}, % C_{TMX}, or % G/Cₜₘₓ. In some embodiments, the guanine content of the ORF of the polynucleotide with respect to the theoretical maximum guanine content of a nucleotide sequence encoding the polypeptide (% G_{TMX}) is at least 69%, at least 70%, at least 75%, at least about 80%, at least about 85%, at least about 90%, at least about 95%, or about 100%. In some embodiments, the % G_{TMx} of the polynucleotide is between about 70% and about 80%, between about 71% and about 79%, between about 71% and about 78%, or between about 71% and about 77%. In some embodiments, the cytosine content of the ORF of the polynucleotide relative to the theoretical maximum cytosine content of a nucleotide sequence encoding the polypeptide (% C_{TMX}) is at least 59%, at least 60%, at least about 65%, at least about 70%, at least about 75%, at least about 80%, at least about 85%, at least about 90%, at least about 95%, or about 100%. In some embodiments, the % C_{TMX} of the ORF of the polynucleotide is between about 60% and about 80%, between about 62% and about 80%, between about 63% and about 79%, or between about 68% and about 76%. In some embodiments, the guanine and cytosine content (G/C) of the ORF of the polynucleotide relative to the theoretical maximum G/C content in a nucleotide sequence encoding the polypeptide (% G/C_{TMX}) is at least about 81%, at least about 85%, at least about 90%, at least about 95%, or about 100%. In some embodiments, the % G/C_{TMx} in the ORF of the polynucleotide is between about 80% and about 100%, between about 85% and about 99%, between about 90% and about 97%, or between about 91% and about 96%. In some embodiments, the G/C content in the ORF of the polynucleotide relative to the G/C content in the corresponding wild-type ORF (% G/C_{WT}) is at least 102%, at least 103%, at least 104%, at least 105%, at least 106%, at least 107%, at least 110%, at least 115%, or at least 120%. In some embodiments, the average G/C content in the 3rd codon position in the ORF of the polynucleotide is at least 20%, at least 21%, at least 22%, at least 23%, at least 24%, at least 25%, at least 26%, at least 27%, at least 28%, at least 29%, or at least 30% higher than the average G/C content in the 3rd codon position in the corresponding wild-type ORF. In some embodiments, the increases in G and/or C content (absolute or relative) described herein can be conducted by replacing synonymous codons with low G, C, or G/C content with synonymous codons having higher G, C, or G/C content. In other embodiments, the increase in G and/or C content (absolute or relative) is conducted by replacing a codon ending with U with a synonymous codon ending with G or C.

In further embodiments, the ORF of the mRNA encoding a polypeptide includes less uracil pairs (UU) and/or uracil triplets (UUU) and/or uracil quadruplets (UUUU) than the corresponding wild-type nucleotide sequence encoding the polypeptide. In some embodiments, the ORF of the mRNA encoding a polypeptide of the disclosure includes no uracil pairs and/or uracil triplets and/or uracil quadruplets. In some embodiments, uracil pairs and/or uracil triplets and/or uracil quadruplets are reduced below a certain threshold, e.g., no more than 1, 2, 3, 4, 5, 6, 7, 8, 9, 10, 11, 12, 13, 14, 15, 16, 17, 18, 19, or 20 occurrences in the ORF of the mRNA encoding the polypeptide. In a particular embodiment, the ORF of the mRNA encoding the polypeptide of the disclosure contains less than 20, 19, 18, 17, 16, 15, 14, 13, 12, 11, 10, 9, 8, 7, 6, 5, 4, 3, 2, or 1 non-phenylalanine uracil pairs and/or triplets. In another embodiment, the ORF of the mRNA encoding the polypeptide contains no non-phenylalanine uracil pairs and/or triplets.

In further embodiments, the ORF of the mRNA encoding a polypeptide of the disclosure includes less uracil-rich clusters than the corresponding wild-type nucleotide sequence encoding the polypeptide. In some embodiments, the ORF of the mRNA encoding the polypeptide of the disclosure contains uracil-rich clusters that are shorter in length than corresponding uracil-rich clusters in the corresponding wild-type nucleotide sequence encoding the polypeptide.

In further embodiments, alternative lower frequency codons are employed. In some embodiment, the ORF of the polynucleotide further comprises at least one low-frequency codon. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the codons in the polypeptide-encoding ORF of the mRNA are substituted with alternative codons, each alternative codon having a codon frequency lower than the codon frequency of the substituted codon in the synonymous codon set. The ORF may also have adjusted uracil content, as described above. In some embodiments, at least one codon in the ORF of the mRNA encoding the polypeptide is substituted with an alternative codon having a codon frequency lower than the codon frequency of the substituted codon in the synonymous codon set.

In some embodiments, the polynucleotide is an mRNA that comprises an ORF that encodes a polypeptide, wherein the uracil content of the ORF is between about 115% and about 135% of the theoretical minimum uracil content in the corresponding wild-type ORF, and wherein the uracil content in the ORF encoding the polypeptide is less than about 30% of the total nucleobase content in the ORF. In some embodiments, the ORF that encodes the polypeptide is further modified to increase G/C content of the ORF (absolute or relative) by at least about 40%, as compared to the corresponding wild-type ORF. In yet other embodiments, the ORF encoding the polypeptide contains less than 20 non-phenylalanine uracil pairs and/or triplets. In some embodiments, at least one codon in the ORF of the mRNA encoding the polypeptide is further substituted with an alternative codon having a codon frequency lower than the codon frequency of the substituted codon in the synonymous codon set.

In some embodiments, the expression of the polypeptide encoded by an mRNA comprising an ORF, wherein the uracil content of the ORF has been adjusted (e.g., the uracil content is between about 115% and about 135% of the theoretical minimum uracil content in the corresponding wild-type ORF) is increased by at least about 10-fold when compared to expression of the polypeptide from the corresponding wild-type mRNA. In some embodiments, the innate immune response induced by the mRNA including an open ORF wherein the uracil content has been adjusted (e.g., the uracil content of the ORF is between about 115% and about 135% of the theoretical minimum uracil content in the corresponding wild-type ORF) is reduced by at least about 10-fold when compared to expression of the polypeptide from the corresponding wild-type mRNA. In some embodiments, the mRNA with adjusted uracil content does not substantially induce an innate immune response of a mammalian cell into which the mRNA is introduced.

In some embodiments, the uracil content of the mRNA is adjusted as described herein, and a modified nucleoside is partially or completely substituted for the uracil remaining in the mRNA following adjustment. As a non-limiting example, the natural nucleotide uridine may be substituted with a modified nucleoside as described herein. In some embodiments, the modified nucleoside comprises pseudouridine (ψ). In some embodiments, the modified nucleoside comprises 1-methyl-pseudouridine (m1ψ). In some embodiments, the modified nucleoside comprises 1-methyl-pseudouridine (m1ψ) and 5-methyl-cytidine (m5C). In some embodiments, the modified nucleoside comprises 2-thiouridine (s2U). In some embodiments, the modified nucleoside comprises 2-thiouridine and 5-methyl-cytidine (m5C). In some embodiments, the modified nucleoside comprises 5-methoxy-uridine (mo5U). In some embodiments, the modified nucleoside comprises 5-methoxy-uridine (mo5U) and 5-methyl-cytidine (m5C). In some embodiments, the modified nucleoside comprises 2′-O-methyl uridine. In some embodiments, the modified nucleoside comprises 2′-O-methyl uridine and 5-methyl-cytidine (m5C). In some embodiments, the modified nucleoside comprises N6-methyl-adenosine (m6A). In some embodiments, the modified nucleoside comprises N6-methyl-adenosine (m6A) and 5-methyl-cytidine (m5C).

Chemical Modification of mRNA

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (Tm5U), 1-taurinomethyl-pseudouridine, 5-taurinomethyl-2-thio-uridine (Tm5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (wm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-0H-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-0-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (mlAm), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m1I), wyo sine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (mlIm), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.) In one embodiment, the modified nucleobase is N1-methylpseudouridine (m1ψ) and the mRNA of the disclosure is fully modified with N1-methylpseudouridine (m1ψ). In some embodiments, N1-methylpseudouridine (m1ψ) represents from 75-100% of the uracils in the mRNA. In some embodiments, N1-methylpseudouridine (m1ψ) represents 100% of the uracils in the mRNA.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (mlw), 5-methoxy-uridine (mo5U), 5-methyl-cytidine (m5C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (ψ). In some embodiments, the mRNA comprises pseudouridine (ψ) and 5-methyl-cytidine (m5C). In some embodiments, the mRNA comprises 1-methyl-pseudouridine (m1ψ). In some embodiments, the mRNA comprises 1-methyl-pseudouridine (m1ψ) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises comprises N6-methyl-adenosine (m6A). In some embodiments, the mRNA comprises N6-methyl-adenosine (m6A) and 5-methyl-cytidine (m5C).

In certain embodiments, an mRNA of the disclosure is uniformly modified (i.e., fully modified, modified through-out the entire sequence) for a particular modification. For example, an mRNA can be uniformly modified with N1-methylpseudouridine (m1ψ) or 5-methyl-cytidine (m5C), meaning that all uridines or all cytosine nucleosides in the mRNA sequence are replaced with N1-methylpseudouridine (m1ψ) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, Calif.) and/or proprietary methods. In one embodiment, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

MicroRNA (miRNA) Binding Sites

Nucleic acid molecules (e.g., RNA, e.g., mRNA) of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof.

In some embodiments, a nucleic acid molecule (e.g., RNA, e.g., mRNA) of the disclosure comprises an open reading frame (ORF) encoding a polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a nucleic acid molecule, e.g., within a DNA or within an RNA transcript, including in the 5′UTR and/or 3′UTR, that has sufficient complementarity to all or a region of a miRNA to interact with, associate with or bind to the miRNA. In some embodiments, a nucleic acid molecule (e.g., RNA, e.g., mRNA) of the disclosure comprising an ORF encoding a polypeptide of interest and further comprises one or more miRNA binding site(s). In exemplary embodiments, a 5′UTR and/or 3′UTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the nucleic acid molecule (e.g., RNA, e.g., mRNA).

For example, one of skill in the art would understand that one or more miR binding sites can be included in a nucleic acid molecule (e.g., an RNA, e.g., mRNA) to minimize expression in cell types other than lymphoid cells. In one embodiment, a miR122 binding site can be used. In another embodiment, a miR126 binding site can be used. In still another embodiment, multiple copies of these miR binding sites or combinations may be used.

Conversely, miRNA binding sites can be removed from nucleic acid molecule (e.g., RNA, e.g., mRNA) sequences in which they naturally occur in order to increase protein expression in specific tissues. For example, a binding site for a specific miRNA can be removed from a nucleic acid molecule (e.g., RNA, e.g., mRNA) to improve protein expression in tissues or cells containing the miRNA.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/1eu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety. Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). Specifically, miRNAs are known to be differentially expressed in immune cells (also called hematopoietic cells), such as antigen presenting cells (APCs) (e.g., dendritic cells and monocytes), monocytes, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a nucleic acid molecule (e.g., RNA, e.g., mRNA) can be shut-off by adding miR-142 binding sites to the 3′-UTR of the polynucleotide, enabling more stable gene transfer in tissues and cells. miR-142 efficiently degrades exogenous nucleic acid molecules (e.g., RNA, e.g., mRNA) in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a nucleic acid molecule of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the nucleic acid molecule (e.g., RNA, e.g., mRNA). The nucleic acid molecule (e.g., RNA, e.g., mRNA) is then stably expressed in target tissues or cells without triggering cytotoxic elimination.

In one embodiment, binding sites for miRNAs that are known to be expressed in immune cells, in particular, antigen presenting cells, can be engineered into a nucleic acid molecule (e.g., RNA, e.g., mRNA) of the disclosure to suppress the expression of the nucleic acid molecule (e.g., RNA, e.g., mRNA) in antigen presenting cells through miRNA mediated RNA degradation, subduing the antigen-mediated immune response. Expression of the nucleic acid molecule (e.g., RNA, e.g., mRNA) is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a nucleic acid molecule of the disclosure.

To further drive the selective degradation and suppression in APCs and macrophage, a nucleic acid molecule (e.g., RNA, e.g., mRNA) of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2--5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p-miR-30e-3p, miR-30e-5p, miR-331-5p, miR-339-3p, miR-339-5p, miR-345-3p, miR-345-5p, miR-346, miR-34a-3p, miR-34a-5p-miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.)

In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′UTR in order to enhance microRNA mediated gene regulation.

At least one miRNA binding site can be engineered into the 3′UTR of a polynucleotide of the disclosure. In this context, at least two, at least three, at least four, at least five, at least six, at least seven, at least eight, at least nine, at least ten, or more miRNA binding sites can be engineered into a 3′UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA) of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a nucleic acid molecule (e.g., RNA, e.g., mRNA) of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition.

In some embodiments, a nucleic acid molecule (e.g., RNA, e.g., mRNA) of the disclosure can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a nucleic acid molecule (e.g., RNA, e.g., mRNA) of the disclosure more unstable in antigen presenting cells. Non-limiting examples of these miRNAs include mir-142-5p, mir-142-3p, mir-146a-5p, and mir-146-3p.

Lipid Nanoparticles

A polynucleotide of the disclosure can be encapsulated in a lipid nanoparticle to facilitate delivery of the polynucleotide sequence into immune cells. Accordingly, in one set of embodiments, lipid nanoparticles (LNPs) are provided. Each of the LNPs described herein may be used as a formulation for mRNA described herein. In one embodiment, a lipid nanoparticle comprises lipids including an ionizable lipid, a sterol or other structural lipid, a non-cationic helper lipid or phospholipid, optionally a PEG lipid, and one or more polynucleotides, e.g., mRNAs.

In certain embodiments, the LNP includes an immune cell delivery potentiating lipid, which promotes delivery of the mRNA into immune cells. In one embodiment, the LNP comprises a phytosterol or a combination of a phytosterol and cholesterol. In one embodiment, the phytosterol is selected from the group consisting of β-sitosterol, stigmasterol, β-sitostanol, campesterol, brassicasterol, and combinations thereof. In one embodiment, the phytosterol is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

Immune Cell Delivery LNPs

Immune cell delivery LNPs can be characterized in that they result in increased delivery of agents to immune cells as compared to a control LNP (e.g., an LNP lacking the immune cell delivery potentiating lipid). In particular, in one embodiment, immune cell delivery LNPs result in an increase (e.g., a 2-fold or more increase) in the percentage of LNPs associated with immune cells as compared to a control LNP or an increase (e.g., a 2-fold or more increase) in the percentage of immune cells expressing the agent carried by the LNP (e.g., expressing the protein encoded by the mRNA associated with/encapsulated by the LNP) as compared to a control LNP. In another embodiment, immune cell delivery LNPs result in increased binding to C1q and/or increased uptake of C1q-bound LNP into the immune cells (e.g., via opsonization) as compared to a control LNP (e.g., an LNP lacking the immune cell delivery potentiating lipid).

In another embodiment, immune cell delivery LNPs result in an increase in the delivery of an agent (e.g., a nucleic acid molecule) to immune cells as compared to a control LNP. In one embodiment, immune cell delivery LNPs result in an increase in the delivery of a nucleic acid molecule agent to T cells as compared to a control LNP. In one embodiment, immune cell delivery LNPs result in an increase in the delivery of a nucleic acid molecule agent to B cells as compared to a control LNP. In one embodiment, immune cell delivery LNPs result in an increase in the delivery of a nucleic acid molecule agent to B cells as compared to a control LNP. In one embodiment, immune cell delivery LNPs result in an increase in the delivery of a nucleic acid molecule agent to myeloid cells as compared to a control LNP.

In one embodiment, when the nucleic acid molecule is an mRNA, an increase in the delivery of a nucleic acid agent to immune cells can be measured by the ability of an LNP to effect at least about 2-fold greater expression of a protein molecule encoded by the mRNA in immune cells, (e.g., T cells) as compared to a control LNP.

Immune cell delivery LNPs comprise an (i) ionizable lipid; (ii) sterol or other structural lipid; (iii) a non-cationic helper lipid or phospholipid; a (iv) PEG lipid and (v) an agent (e.g., a nucleic acid molecule) encapsulated in and/or associated with the LNP, wherein one or more of (i) the ionizable lipid or (ii) the structural lipid or sterol in an immune cell delivery LNPs comprises an effective amount of an immune cell delivery potentiating lipid.

In another embodiment, an immune cell delivery lipid nanoparticle of the disclosure comprises:

(i) an ionizable lipid;

(ii) a sterol or other structural lipid;

(iii) a non-cationic helper lipid or phospholipid;

(iv) an agent for delivery to an immune cell, and

(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to an immune cell. In one embodiment, enhanced delivery is relative to a lipid nanoparticle lacking the immune cell delivery potentiating lipid. In another embodiment, the enhanced delivery is relative to a suitable control.

In another embodiment, an immune cell delivery lipid nanoparticle of the disclosure comprises:

(i) an ionizable lipid;

(ii) a sterol or other structural lipid;

(iii) a non-cationic helper lipid or phospholipid;

(iv) an agent for delivery to an immune cell, and

(v) optionally, a PEG-lipid

wherein one or more of (i) the ionizable lipid or (ii) the sterol or other structural lipid or (iii) the non-cationic helper lipid or phospholipid or (v) the PEG lipid is a C1q binding lipid that binds to C1q or promotes (e.g., increases, stimulates, enhances) the binding of the LNP to C1q, as compared to a control LNP lacking the C1q binding lipid.

In another embodiment, an immune cell delivery lipid nanoparticle of the disclosure comprises:

(i) an ionizable lipid;

(ii) a sterol or other structural lipid;

(iii) a non-cationic helper lipid or phospholipid;

(iv) an agent for delivery to an immune cell, and

(v) optionally, a PEG-lipid

wherein one or more of (i) the ionizable lipid or (ii) the sterol or other structural lipid binds to C1q or promotes (e.g., increases, stimulates, enhances) the binding of the LNP to C1q, as compared to a control LNP (e.g., an LNP lacking (i) the ionizable lipid or (ii) the sterol or other structural lipid).

In another aspect, the disclosure provides a method of screening for an immune cell delivery lipid, the method comprising contacting a test LNP comprising a test immune cell delivery lipid with C1q, and measuring binding to C1q, wherein a test immune cell delivery lipid is selected as an immune cell delivery lipid when it binds to C1q or promotes (e.g., increases, stimulates, enhances) the binding of the LNP comprising it to C1q.

### Lipid Content of LNPs

As set forth above, with respect to lipids, immune cell delivery LNPs comprise an (i) ionizable lipid; (ii) sterol or other structural lipid; (iii) a non-cationic helper lipid or phospholipid; a (iv) PEG lipid, wherein one or more of (i) the ionizable lipid or (ii) the structural lipid or sterol in an immune cell delivery LNPs comprises an effective amount of an immune cell delivery potentiating lipid. These categories of lipids are set forth in more detail below.

(i) Ionizable Lipids

The lipid nanoparticles of the present disclosure include one or more ionizable lipids. In certain embodiments, the ionizable lipids of the disclosure comprise a central amine moiety and at least one biodegradable group. The ionizable lipids described herein may be advantageously used in lipid nanoparticles of the disclosure for the delivery of nucleic acid molecules to mammalian cells or organs. The structures of ionizable lipids set forth below include the prefix I to distinguish them from other lipids of the disclosure.

In a first aspect of the disclosure, the compounds described herein are of Formula (I I):

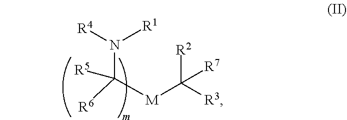

or their N-oxides, or salts or isomers thereof, wherein:

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═N R⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected

from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl;

each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13; and wherein when R⁴ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

Another aspect the disclosure relates to compounds of Formula (III):

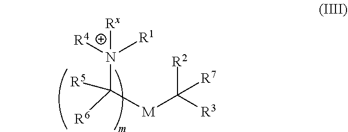

or its N-oxide, or a salt or isomer thereof, wherein

or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)OC(R¹⁰)₂(CH₂)ₙ₋ₒQ, —CHQR, —CQ(R)₂, and unsubstituted C₁-6 alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

R^{x} is selected from the group consisting of C₁₋₆ alkyl, C₂₋₆ alkenyl, —(CH₂)ᵥO H, and —(CH₂)ᵥN(R)₂,

wherein v is selected from 1, 2, 3, 4, 5, and 6;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl;

each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

In certain embodiments, a subset of compounds of Formula (I) includes those of Formula (IA):

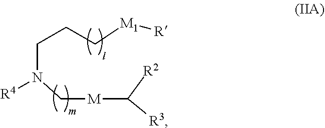

or its N-oxide, or a salt or isomer thereof, wherein 1 is selected from 1, 2, 3, 4, and 5; m is selected from 5, 6, 7, 8, and 9; M₁ is a bond or M′; R⁴ is hydrogen, unsubstituted C₁₋₃ alkyl, —(CH₂)OC(R¹⁰)₂(CH₂)ₙ₋ₒQ, or —(CH₂)ₙQ, in which Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group, and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

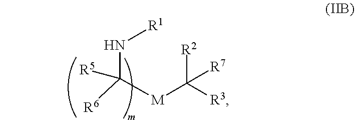

or its N-oxide, or a salt or isomer thereof in which all variables are as defined herein. For example, m is selected from 5, 6, 7, 8, and 9; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. In certain embodiments, a subset of compounds of Formula (I) includes those of Formula (II):

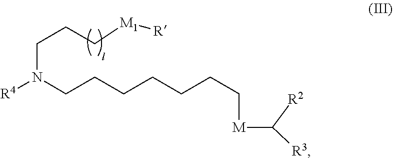

or its N-oxide, or a salt or isomer thereof, wherein 1 is selected from 1, 2, 3, 4, and 5; M₁ is a bond or M′; R⁴ is hydrogen, unsubstituted C₁-3 alkyl, —(CH₂)OC(R¹⁰)₂(CH₂)ₙ₋ₒQ, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected

from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl.

Another aspect of the disclosure relates to compounds of Formula (I VI):

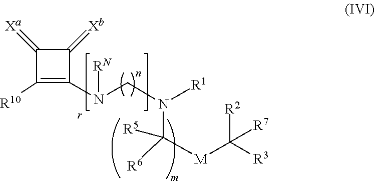

or its N-oxide, or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

each R is independently selected from the group consisting of H, C₁₋₃ alkyl, and C₂₋₃ alkenyl;

R^{N} is H, or C₁₋₃ alkyl;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I;

X^{a} and X^{b} are each independently O or S;

R¹⁰ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₜ₁N(R)₂, —NH(CH₂)ₚ₁O (CH₂)_{q1}N(R)₂, —NH(CH₂)ₛ₁OR, —N((CH₂)ₛ₁OR)₂, a carbocycle, a heterocycle, aryl and heteroaryl;

m is selected from 5, 6, 7, 8, 9, 10, 11, 12, and 13;

n is selected from 1, 2, 3, 4, 5, 6, 7, 8, 9, and 10;

r is 0 or 1;

t¹ is selected from 1, 2, 3, 4, and 5;

p¹ is selected from 1, 2, 3, 4, and 5;

q¹ is selected from 1, 2, 3, 4, and 5; and

s¹ is selected from 1, 2, 3, 4, and 5.

In one embodiment, a subset of compounds of Formula (VI) includes those of Formula (VI-a):

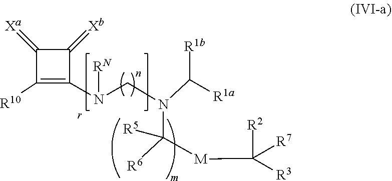

or its N-oxide, or a salt or isomer thereof, wherein

R^{1a} and R^{1b} are independently selected from the group consisting of C₁₋₁₄ alkyl and C₂₋₁₄ alkenyl; and

R² and R³ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle.

In another embodiment, a subset of compounds of Formula (VI) includes those of Formula (VII):

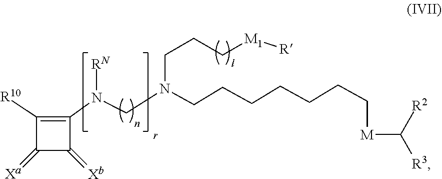

or its N-oxide, or a salt or isomer thereof, wherein

1 is selected from 1, 2, 3, 4, and 5;

M₁ is a bond or M′; and

R² and R³ are independently selected from the group consisting of H, C₁₋₁₄ alkyl, and C₂₋₁₄ alkenyl.

In another embodiment, a subset of compounds of Formula (I VI) includes those of Formula (I VIII):

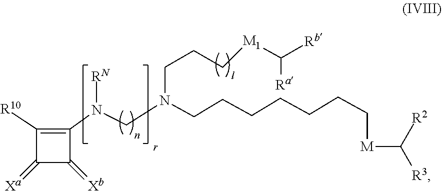

or its N-oxide, or a salt or isomer thereof, wherein

1 is selected from 1, 2, 3, 4, and 5;

M₁ is a bond or M′; and

R^{a′} and R^{b′} are independently selected from the group consisting of C₁₋₁₄ alkyl and C₂₋₁₄ alkenyl; and

R² and R³ are independently selected from the group consisting of C₁₋₁₄ alkyl, and C₂₋₁₄ alkenyl.

The compounds of any one of formula (I I), (I IA), (I VI), (I VI-a), (I VII) or (I VIII) include one or more of the following features when applicable.

In some embodiments, M₁ is M′.

In some embodiments, M and M′ are independently —C(O)O— or —OC(O)—.

In some embodiments, at least one of M and M′ is —C(O)O— or —OC(O)—.

In certain embodiments, at least one of M and M′ is —OC(O)—.

In certain embodiments, M is —OC(O)— and M′ is —C(O)O—. In some embodiments, M is —C(O)O— and M′ is —OC(O)—. In certain embodiments, M and M′ are each —OC(O)—. In some embodiments, M and M′ are each —C(O)O—.

In certain embodiments, at least one of M and M′ is —OC(O)-M″-C(O)O—.

In some embodiments, M and M′ are independently —S—S—.

In some embodiments, at least one of M and M′ is —S—S.

In some embodiments, one of M and M′ is —C(O)O— or —OC(O)— and the other is —S—S—. For example, M is —C(O)O— or —OC(O)— and M′ is —S—S— or M′ is —C(O)O—, or —OC(O)— and M is —S—S—.

In some embodiments, one of M and M′ is —OC(O)-M″-C(O)O—, in which M″ is a bond, C₁₋₁₃ alkyl or C₂₋₁₃ alkenyl. In other embodiments, M″ is C₁₋₆ alkyl or C₂₋₆ alkenyl. In certain embodiments, M″ is C₁₋₄ alkyl or C₂₋₄ alkenyl. For example, in some embodiments, M″ is C₁ alkyl. For example, in some embodiments, M″ is C₂ alkyl. For example, in some embodiments, M″ is C₃ alkyl. For example, in some embodiments, M″ is C₄ alkyl. For example, in some embodiments, M″ is C₂ alkenyl. For example, in some embodiments, M″ is C₃ alkenyl. For example, in some embodiments, M″ is C₄ alkenyl.

In some embodiments, 1 is 1, 3, or 5.

In some embodiments, R⁴ is hydrogen.

In some embodiments, R⁴ is not hydrogen.

In some embodiments, R⁴ is unsubstituted methyl or —(CH₂)ₙQ, in which Q is OH, —NHC(S)N(R)₂, —NHC(O)N(R)₂, —N(R)C(O)R, or —N(R)S(O)₂R.

In some embodiments, Q is OH.

In some embodiments, Q is —NHC(S)N(R)₂.

In some embodiments, Q is —NHC(O)N(R)₂.

In some embodiments, Q is —N(R)C(O)R.

In some embodiments, Q is —N(R)S(O)₂R.

In some embodiments, Q is —O(CH₂)ₙN(R)₂.

In some embodiments, Q is —O(CH₂)ₙOR.

In some embodiments, Q is —N(R)R⁸.

In some embodiments, Q is —NHC(═NR⁹)N(R)₂.

In some embodiments, Q is —NHC(═CHR⁹)N(R)₂.

In some embodiments, Q is —OC(O)N(R)₂.

In some embodiments, Q is —N(R)C(O)OR.

In some embodiments, n is 2.

In some embodiments, n is 3.

In some embodiments, n is 4.

In some embodiments, M₁ is absent.

In some embodiments, at least one R⁵ is hydroxyl. For example, one R⁵ is hydroxyl.

In some embodiments, at least one R⁶ is hydroxyl. For example, one R⁶ is hydroxyl.

In some embodiments one of R⁵ and R⁶ is hydroxyl. For example, one R⁵ is hydroxyl and each R⁶ is hydrogen. For example, one R⁶ is hydroxyl and each R⁵ is hydrogen.

In some embodiments, R^{x} is C₁₋₆ alkyl. In some embodiments, R^{x} is C₁₋₃ alkyl. For example, R^{x} is methyl. For example, R^{x} is ethyl. For example, R^{x} is propyl.

In some embodiments, R^{x} is —(CH₂)ᵥOH and, v is 1, 2 or 3. For example, R^{x} is methanoyl. For example, R^{x} is ethanoyl. For example, R^{x} is propanoyl.

In some embodiments, R′ is —(CH₂)ᵥN(R)₂, v is 1, 2 or 3 and each R is H or methyl. For example, R′ is methanamino, methylmethanamino, or dimethylmethanamino. For example, R′ is aminomethanyl, methylaminomethanyl, or dimethylaminomethanyl. For example, R′ is aminoethanyl, methylaminoethanyl, or dimethylaminoethanyl. For example, R′ is aminopropanyl, methylaminopropanyl, or dimethylaminopropanyl.

In some embodiments, R′ is C₁₋₁₈ alkyl, C₂₋₁₈ alkenyl, —R*YR″, or —YR″.

In some embodiments, R² and R³ are independently C₃₋₁₄ alkyl or C₃₋₁₄ alkenyl.

In some embodiments, R^{1b} is C₁₋₁₄ alkyl. In some embodiments, R^{1b} is C₂₋₁₄ alkyl. In some embodiments, R^{1b} is C₃₋₁₄ alkyl. In some embodiments, R^{1b} is C₁₋₈ alkyl. In some embodiments, R^{1b} is C₁₋₅ alkyl. In some embodiments, R^{1b} is C₁₋₃ alkyl. In some embodiments, R^{1b} is selected from C₁ alkyl, C₂ alkyl, C₃ alkyl, C₄ alkyl, and C₅ alkyl. For example, in some embodiments, R^{1b} is C₁ alkyl. For example, in some embodiments, R^{1b} is C₂ alkyl. For example, in some embodiments, R^{1b} is C₃ alkyl. For example, in some embodiments, R^{1b} is C₄ alkyl. For example, in some embodiments, R^{1b} is C₅ alkyl.

In some embodiments, R¹ is different from —(CHR⁵R⁶)ₘ-M-CR²R³R⁷.

In some embodiments, —CHR^{1a}R^{1b} is different from —(CHR⁵R⁶)ₘ-M-CR²R³R⁷.

In some embodiments, R⁷ is H. In some embodiments, R⁷ is selected from C₁₋₃ alkyl. For example, in some embodiments, R⁷ is C₁ alkyl. For example, in some embodiments, R⁷ is C₂ alkyl. For example, in some embodiments, R⁷ is C₃ alkyl. In some embodiments, R⁷ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and Cis alkenyl.

In some embodiments, R^{b′} is C₁₋₁₄ alkyl. In some embodiments, R^{b′} is C₂₋₁₄ alkyl. In some embodiments, R^{b′} is C₃₋₁₄ alkyl. In some embodiments, R^{b′} is C₁₋₈ alkyl. In some embodiments, R^{b′} is C₁₋₅ alkyl. In some embodiments, R^{b′} is C₁-3 alkyl. In some embodiments, R^{b′} is selected from C₁ alkyl, C₂ alkyl, C₃ alkyl, C₄ alkyl and C₅ alkyl. For example, in some embodiments, R^{b′} is C₁ alkyl. For example, in some embodiments, R^{b′} is C₂ alkyl. For example, some embodiments, R^{b′} is C₃ alkyl. For example, some embodiments, R^{b′} is C₄ alkyl.

In one embodiment, the compounds of Formula (I) are of Formula (IIa):

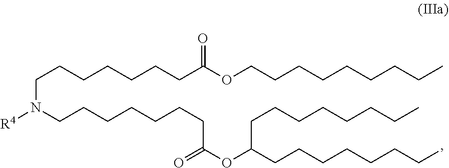

or their N-oxides, or salts or isomers thereof, wherein R⁴ is as described herein. In another embodiment, the compounds of Formula (I) are of Formula (IIb):

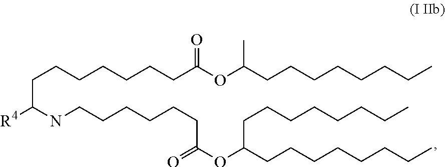

or their N-oxides, or salts or isomers thereof, wherein R⁴ is as described herein. In another embodiment, the compounds of Formula (I) are of Formula (IIc) or (IIe):

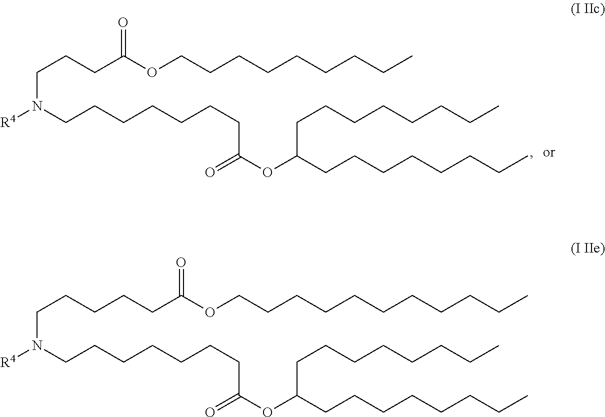

or their N-oxides, or salts or isomers thereof, wherein R⁴ is as described herein. In another embodiment, the compounds of Formula (I I) are of Formula (I IIf):

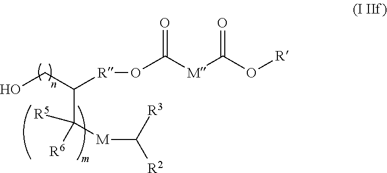

or their N-oxides, or salts or isomers thereof,

wherein M is —C(O)O— or —OC(O)—, M″ is C₁₋₆ alkyl or C₂₋₆ alkenyl, R² and R³ are independently selected from the group consisting of C₅₋₁₄ alkyl and C₅₋₁₄ alkenyl, and n is selected from 2, 3, and 4.

In a further embodiment, the compounds of Formula (I I) are of Formula (IId):

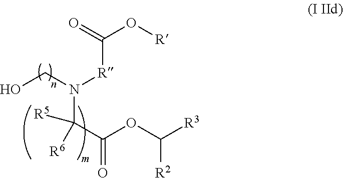

or their N-oxides, or salts or isomers thereof, wherein n is 2, 3, or 4; and m, R′, R″, and R² through R6 are as described herein. For example, each of R² and R³ may be independently selected from the group consisting of C₅₋₁₄ alkyl and C₅₋₁₄ alkenyl.

In a further embodiment, the compounds of Formula (I) are of Formula (IIg):

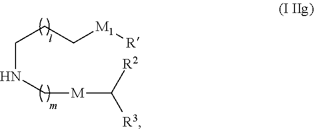

or their N-oxides, or salts or isomers thereof, wherein 1 is selected from 1, 2, 3, 4, and 5; m is selected from 5, 6, 7, 8, and 9; M₁ is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R² and R³ are independently selected from the group consisting of C₅₋₁₄ alkyl and C₅₋₁₄ alkenyl.

In another embodiment, a subset of compounds of Formula (I VI) includes those of Formula (I VIIa):

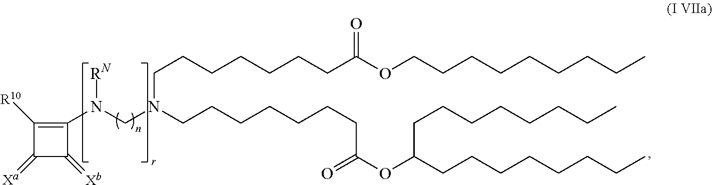

or its N-oxide, or a salt or isomer thereof.

In another embodiment, a subset of compounds of Formula (I VI) includes those of Formula (I VIIIa):

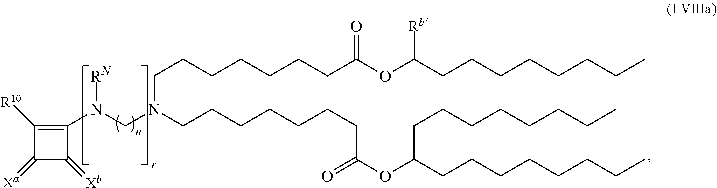

or its N-oxide, or a salt or isomer thereof.

In another embodiment, a subset of compounds of Formula (I VI) includes those of Formula (I VIIIb):

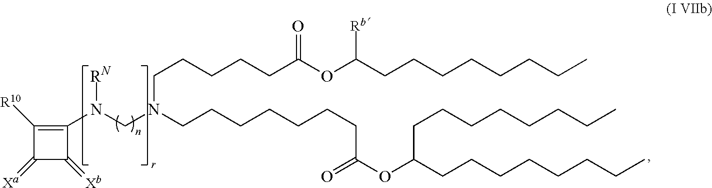

or its N-oxide, or a salt or isomer thereof.

In another embodiment, a subset of compounds of Formula (I VI) includes those of Formula (I VIIb-1):

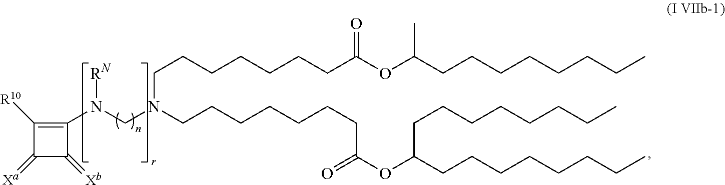

or its N-oxide, or a salt or isomer thereof.

In another embodiment, a subset of compounds of Formula (I VI) includes those of Formula (I VIIb-2):

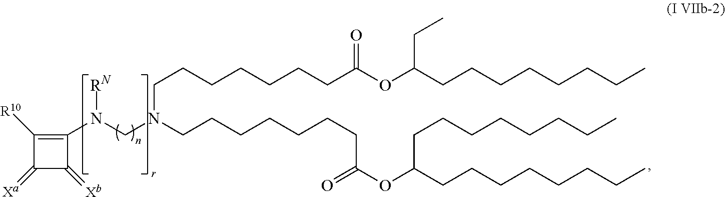

or its N-oxide, or a salt or isomer thereof.

In another embodiment, a subset of compounds of Formula (I VI) includes those of Formula (I VIIb-3):

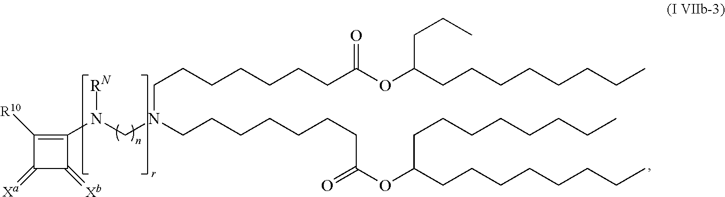

or its N-oxide, or a salt or isomer thereof. In another embodiment, a subset of compounds of Formula (VI) includes those of Formula (VIIc):

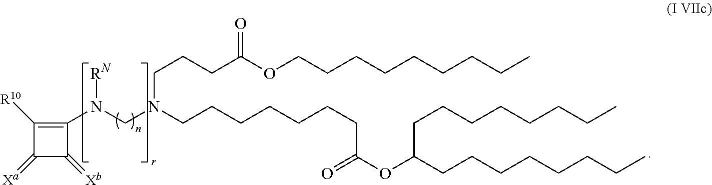

In another embodiment, a subset of compounds of Formula (I VI) includes those of Formula (VIId):

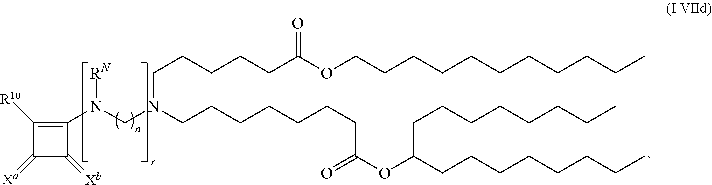

or its N-oxide, or a salt or isomer thereof.

In another embodiment, a subset of compounds of Formula (I VI) includes those of Formula (I VIIIc):

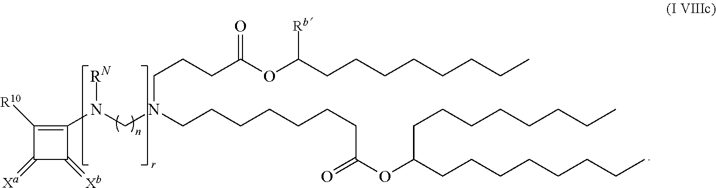

In another embodiment, a subset of compounds of Formula I VI) includes those of Formula (I VIIId):

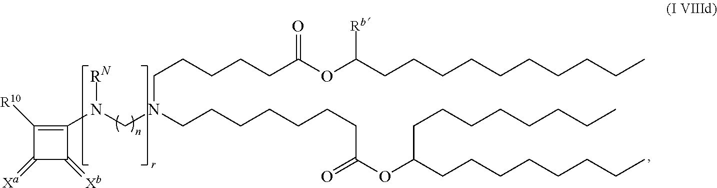

or its N-oxide, or a salt or isomer thereof.

The compounds of any one of formulae (I I), (I IA), (I IB), (I II), (I IIa), (I IIb), (I IIc), (I IId), (I IIe), (I IIf), (I IIg), I (III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), or (I VIIId) include one or more of the following features when applicable.

In some embodiments, R⁴ is selected from the group consisting of a C₃-6 carbocycle, —(CH₂)ₙQ, —(CH₂)ₙCHQR, —(CH₂)OC(R¹⁰)₂(CH₂)ₙ₋ₒQ, —CHQR, and —CQ(R)₂, where Q is selected from a C₃₋₆ carbocycle, 5- to 14-membered aromatic or non-aromatic heterocycle having one or more heteroatoms selected from N, O, S, and P, —OR, —O(CH₂)ₙN(R)₂, —C(O)OR, —OC(O)R, —CX₃, —CX₂H, —CXH₂, —CN, —N(R)₂, —N(R)S(O)₂R⁸, —C(O)N(R)₂, —N(R)C(O)R, —N(R)S(O)₂R, —N(R)C(O)N(R)₂, —N(R)C(S)N(R)₂, and —C(R)N(R)₂C(O)OR, each o is independently selected from 1, 2, 3, and 4, and each n is independently selected from 1, 2, 3, 4, and 5.

In another embodiment, R⁴ is selected from the group consisting of a C₃-6 carbocycle, —(CH₂)ₙQ, —(CH₂)ₙCHQR, —(CH₂)OC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR, —(CH₂)OC(R¹⁰)₂(CH₂)ₙ₋ₒQ, —CHQR, and —CQ(R)₂, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5; and when Q is a 5- to 14-membered heterocycle and (i) R⁴ is —(CH₂)ₙQ in which n is 1 or 2, or (ii) R⁴ is —(CH₂)ₙCHQR in which n is 1, or (iii) R⁴ is —CHQR, and —CQ(R)₂, then Q is either a 5- to 14-membered heteroaryl or 8- to 14-membered heterocycloalkyl.

In another embodiment, R⁴ is selected from the group consisting of a C₃-6 carbocycle, —(CH₂)ₙQ, —(CH₂)ₙCHQR, —(CH₂)OC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5.

In another embodiment, R⁴ is —(CH₂)ₙQ, where Q is —N(R)S(O)₂R⁸ and n is selected from 1, 2, 3, 4, and 5. In a further embodiment, R⁴ is —(CH₂)ₙQ, where Q is —N(R)S(O)₂R⁸, in which R⁸ is a C₃₋₆ carbocycle such as C₃₋₆ cycloalkyl, and n is selected from 1, 2, 3, 4, and 5. For example, R⁴ is —(CH₂)₃NHS(O)₂R⁸ and R⁸ is cyclopropyl.

In another embodiment, R⁴ is —(CH₂)OC(R¹⁰)₂(CH₂)ₙ₋ₒQ, where Q is —N(R)C(O)R, n is selected from 1, 2, 3, 4, and 5, and o is selected from 1, 2, 3, and 4. In a further embodiment, R⁴ is —(CH₂)OC(R¹⁰)₂(CH₂)ₙ₋ₒQ, where Q is —N(R)C(O)R, wherein R is C₁-C₃ alkyl and n is selected from 1, 2, 3, 4, and 5, and o is selected from 1, 2, 3, and 4. In a another embodiment, R⁴ is is —(CH₂)OC(R¹⁰)₂(CH₂)ₙ₋ₒQ, where Q is —N(R)C(O)R, wherein R is C₁-C₃ alkyl, n is 3, and o is 1. In some embodiments, R¹⁰ is H, OH, C₁₋₃ alkyl, or C₂₋₃ alkenyl. For example, R⁴ is 3-acetamido-2,2-dimethylpropyl.

In some embodiments, one R¹⁰ is H and one R¹⁰ is C₁₋₃ alkyl or C₂₋₃ alkenyl. In another embodiment, each R¹⁰ is is C₁₋₃ alkyl or C₂₋₃ alkenyl. In another embodiment, each R¹⁰ is is C₁₋₃ alkyl (e.g. methyl, ethyl or propyl). For example, one R¹⁰ is methyl and one R¹⁰ is ethyl or propyl. For example, one R¹⁰ is ethyl and one R¹⁰ is methyl or propyl. For example, one R¹⁰ is propyl and one R¹⁰ is methyl or ethyl. For example, each R¹⁰ is methyl. For example, each R¹⁰ is ethyl. For example, each R¹⁰ is propyl.

In some embodiments, one R¹⁰ is H and one R¹⁰ is OH. In another embodiment, each R¹⁰ is is OH.

In another embodiment, R⁴ is unsubstituted C₁₋₄ alkyl, e.g., unsubstituted methyl.

In another embodiment, R⁴ is hydrogen.

In certain embodiments, the disclosure provides a compound having the Formula (I), wherein R⁴ is —(CH₂)ₙQ or —(CH₂)ₙCHQR, where Q is —N(R)₂, and n is selected from 3, 4, and 5.

In certain embodiments, the disclosure provides a compound having the Formula (I), wherein R⁴ is selected from the group consisting of —(CH₂)ₙQ, —(CH₂)ₙCHQR, —CHQR, and —CQ(R)₂, where Q is —N(R)₂, and n is selected from 1, 2, 3, 4, and 5.

In certain embodiments, the disclosure provides a compound having the Formula (I), wherein R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle, and R⁴ is —(CH₂)ₙQ or —(CH₂)ₙCHQR, where Q is —N(R)₂, and n is selected from 3, 4, and 5.

In certain embodiments, R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle. In some embodiments, R² and R³ are independently selected from the group consisting of C₂₋₁₄ alkyl, and C₂₋₁₄ alkenyl. In some embodiments, R² and R³ are independently selected from the group consisting of —R*YR″, —YR″, and —R*OR″. In some embodiments, R² and R³ together with the atom to which they are attached, form a heterocycle or carbocycle.

In some embodiments, R¹ is selected from the group consisting of C₅₋₂₀ alkyl and C₅₋₂₀ alkenyl. In some embodiments, R¹ is C₅₋₂₀ alkyl substituted with hydroxyl.

In other embodiments, R¹ is selected from the group consisting of —R*YR″, —YR″, and —R″M′R′.

In certain embodiments, R¹ is selected from —R*YR″ and —YR″. In some embodiments, Y is a cyclopropyl group. In some embodiments, R* is C₈ alkyl or C₈ alkenyl. In certain embodiments, R″ is C₃₋₁₂ alkyl. For example, R″ may be C₃ alkyl. For example, R″ may be C₄₋₈ alkyl (e.g., C₄, C₅, C₆, C₇, or C₈ alkyl).

In some embodiments, R is (CH₂)_{q}OR*, q is selected from 1, 2, and 3, and R* is C₁₋₁₂ alkyl substituted with one or more substituents selected from the group consisting of amino, C₁-C₆ alkylamino, and C₁-C₆ dialkylamino. For example, R is (CH₂)_{q}OR*, q is selected from 1, 2, and 3 and R* is C₁₋₁₂ alkyl substituted with C₁-C₆ dialkylamino. For example, R is (CH₂)_{q}OR*, q is selected from 1, 2, and 3 and R* is C₁₋₃ alkyl substituted with C₁-C₆ dialkylamino. For example, R is (CH₂)_{q}OR*, q is selected from 1, 2, and 3 and R* is C₁₋₃ alkyl substituted with dimethylamino (e.g., dimethylaminoethanyl).

In some embodiments, R¹ is C₅₋₂₀ alkyl. In some embodiments, R¹ is C₆ alkyl. In some embodiments, R¹ is C₈ alkyl. In other embodiments, R¹ is C₉ alkyl. In certain embodiments, R¹ is C₁₄ alkyl. In other embodiments, R¹ is C₁₈ alkyl.

In some embodiments, R¹ is C₂₁₋₃₀ alkyl. In some embodiments, R¹ is C₂₆ alkyl. In some embodiments, R¹ is C₂₈ alkyl. In certain embodiments, R¹ is

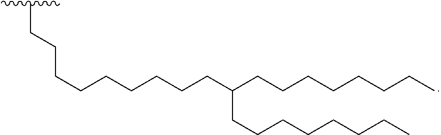 [SMILES: *CCCCCCCCCC(CCCCCCCC)CCCCCCCC]

In some embodiments, R¹ is C₅₋₂₀ alkenyl. In certain embodiments, R¹ is C₁₈ alkenyl. In some embodiments, R¹ is linoleyl.

In certain embodiments, R¹ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl, or heptadeca-9-yl). In certain embodiments, R¹ is

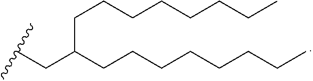 [SMILES: *CC(CCCCCCCC)CCCCCCCC]

In certain embodiments, R¹ is unsubstituted C₅₋₂₀ alkyl or C₅₋₂₀ alkenyl. In certain embodiments, R′ is substituted C₅₋₂₀ alkyl or C₅₋₂₀ alkenyl (e.g., substituted with a C₃₋₆ carbocycle such as 1-cyclopropylnonyl or substituted with OH or alkoxy). For example, R¹ is

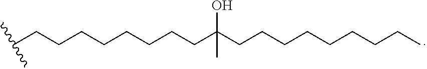 [SMILES: *CCCCCCCCC(C)(O)CCCCCCCCC]

In other embodiments, R¹ is —R″M′R′. In certain embodiments, M′ is —OC(O)-M″-C(O)O—. For example, R¹ is

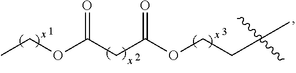

wherein x¹ is an integer between 1 and 13 (e.g., selected from 3, 4, 5, and 6), x² is an integer between 1 and 13 (e.g., selected from 1, 2, and 3), and x³ is an integer between 2 and 14 (e.g., selected from 4, 5, and 6). For example, x¹ is selected from 3, 4, 5, and 6, x² is selected from 1, 2, and 3, and x³ is selected from 4, 5, and 6.

In other embodiments, R¹ is different from —(CHR⁵R⁶)ₘ-M-CR²R³R⁷.

In some embodiments, R′ is selected from —R*YR″ and —YR″. In some embodiments, Y is C₃₋₈ cycloalkyl. In some embodiments, Y is C₆₋₁₀ aryl. In some embodiments, Y is a cyclopropyl group. In some embodiments, Y is a cyclohexyl group. In certain embodiments, R* is C₁ alkyl.

In some embodiments, R″ is selected from the group consisting of C₃₋₁₂ alkyl and C₃₋₁₂ alkenyl. In some embodiments, R″ is C₈ alkyl. In some embodiments, R″ adjacent to Y is C₁ alkyl. In some embodiments, R″ adjacent to Y is C₄₋₉ alkyl (e.g., C₄, C₅, C₆, C₇ or C₈ or C₉ alkyl).

In some embodiments, R″ is substituted C₃₋₁₂ (e.g., C₃₋₁₂ alkyl substituted with, e.g., an hydroxyl). For example, R″ is

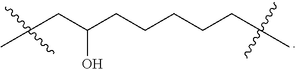 [SMILES: *CCCCCC(O)C*]

In some embodiments, R′ is selected from C₄ alkyl and C₄ alkenyl. In certain embodiments, R′ is selected from C₅ alkyl and C₅ alkenyl. In some embodiments, R′ is selected from C₆ alkyl and C₆ alkenyl. In some embodiments, R′ is selected from C₇ alkyl and C₇ alkenyl. In some embodiments, R′ is selected from C₉ alkyl and C₉ alkenyl.

In some embodiments, R′ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl, each of which is either linear or branched.

In some embodiments, R′ is linear. In some embodiments, R′ is branched.

In some embodiments, R′ is

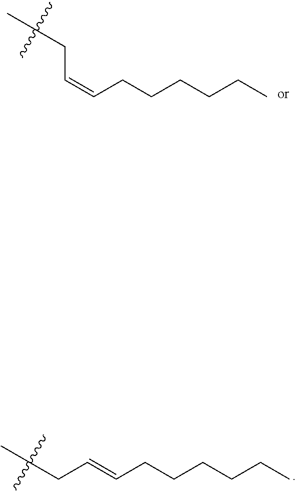

In some embodiments, R′ is

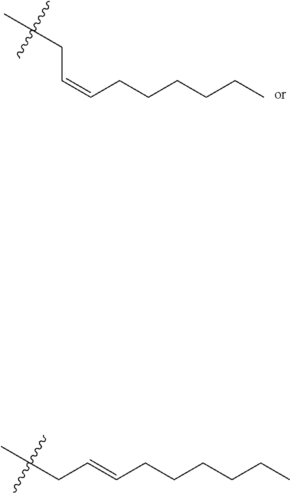

and M′ is —OC(O)—. In other embodiments, R′ is

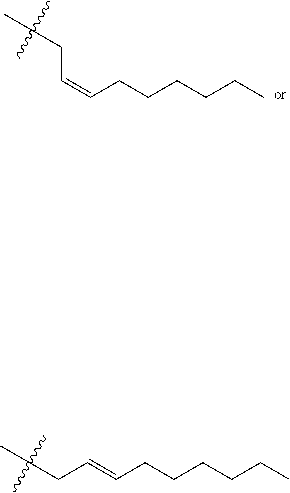

### and M′ is —C(O)O—.

In other embodiments, R′ is selected from C₁₁ alkyl and C₁₁ alkenyl. In other embodiments, R′ is selected from C₁₁ alkyl, C₁₁ alkenyl, C₁₃ alkyl, C₁₃ alkenyl, C₁₄ alkyl, C₁₄ alkenyl, C₁₅ alkyl, Cis alkenyl, C₁₆ alkyl, C₁₆ alkenyl, C₁₇ alkyl, C₁₇ alkenyl, C₁₈ alkyl, and C₁₈ alkenyl. In certain embodiments, R′ is linear C₄₋₁₈ alkyl or C₄₋₁₈ alkenyl. In certain embodiments, R′ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl or heptadeca-9-yl). In certain embodiments, R′ is

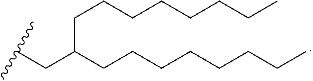 [SMILES: *CC(CCCCCCCC)CCCCCCCC]

In certain embodiments, R′ is unsubstituted C₁-18 alkyl. In certain embodiments, R′ is substituted C₁-18 alkyl (e.g., C₁₋₁₅ alkyl substituted with, e.g., an alkoxy such as methoxy, or a C₃-6 carbocycle such as 1-cyclopropylnonyl, or C(O)O-alkyl or OC(O)-alkyl such as C(O)OCH₃ or OC(O)CH₃). For example, R′ is

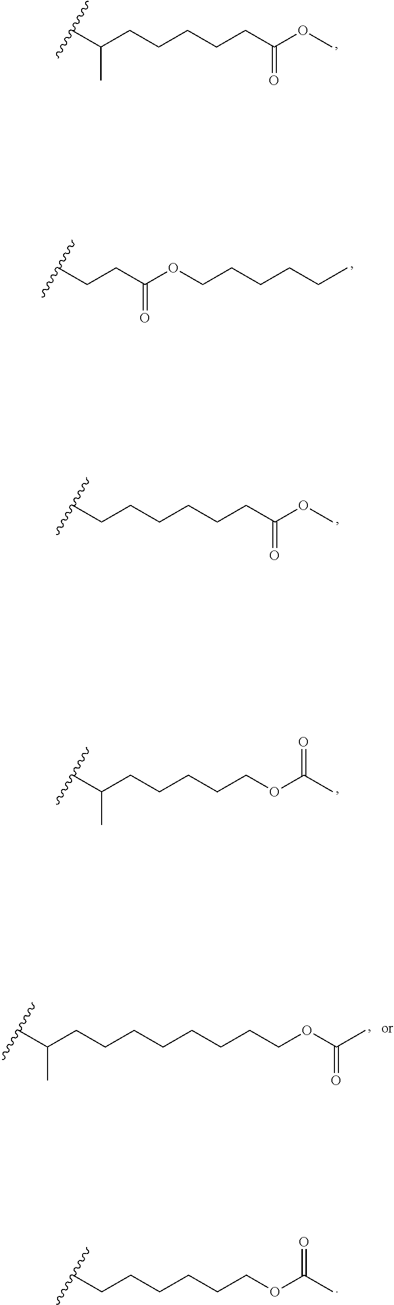 [SMILES: *C(C)CCCCCC(=O)OC ; *CCC(=O)OCCCCCC ; *CCCCCCC(=O)OC ; *C(C)CCCCCOC(C)=O ; *C(C)CCCCCCCCOC(C)=O ; *CCCCCCOC(C)=O]

In certain embodiments, R′ is branched C₁₋₁₈ alkyl. For example, R′ is

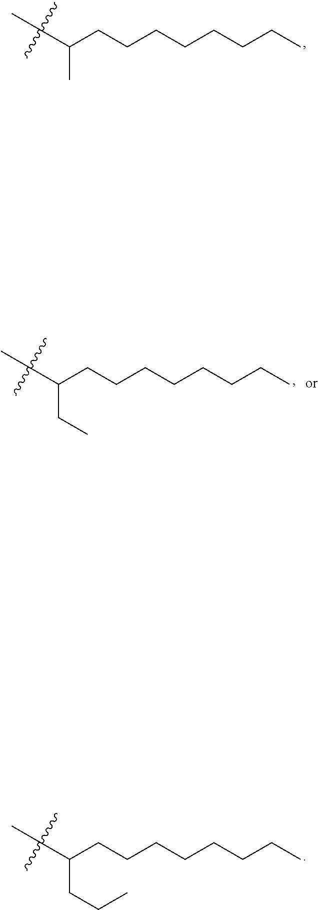 [SMILES: *C(C)CCCCCCCC ; *C(CC)CCCCCCCC ; *C(CCC)CCCCCCCC]

In some embodiments, R″ is selected from the group consisting of C₃₋₁₅ alkyl and C₃₋₁₅ alkenyl. In some embodiments, R″ is C₃ alkyl, C₄ alkyl, C₅ alkyl, C₆ alkyl, C₇ alkyl, or C₈ alkyl. In some embodiments, R″ is C₉ alkyl, Cm alkyl, C₁₁ alkyl, C₁₂ alkyl, C₁₃ alkyl, C₁₄ alkyl, or Cis alkyl.

In some embodiments, M′ is —C(O)O—. In some embodiments, M′ is —OC(O)—. In some embodiments, M′ is —OC(O)-M″-C(O)O—.

In some embodiments, M′ is —C(O)O—, —OC(O)—, or —OC(O)-M″-C(O)O—. In some embodiments wherein M′ is —OC(O)-M″-C(O)O—, M″ is C₁₋₄ alkyl or C₂₋₄ alkenyl.

In other embodiments, M′ is an aryl group or heteroaryl group. For example, M′ may be selected from the group consisting of phenyl, oxazole, and thiazole.

In some embodiments, M is —C(O)O—. In some embodiments, M is —OC(O)—. In some embodiments, M is —C(O)N(R′)—. In some embodiments, M is —P(O)(OR′)O—. In some embodiments, M is —OC(O)-M″-C(O)O—.

In some embodiments, M is —C(O). In some embodiments, M is —OC(O)— and M′ is —C(O)O—. In some embodiments, M is —C(O)O— and M′ is —OC(O)—. In some embodiments, M and M′ are each —OC(O)—. In some embodiments, M and M′ are each —C(O)O—.

In other embodiments, M is an aryl group or heteroaryl group. For example, M may be selected from the group consisting of phenyl, oxazole, and thiazole.

In some embodiments, M is the same as M′. In other embodiments, M is different from M′.

In some embodiments, M″ is a bond. In some embodiments, M″ is C₁₋₁₃ alkyl or C₂₋₁₃ alkenyl. In some embodiments, M″ is C₁₋₆ alkyl or C₂₋₆ alkenyl. In certain embodiments, M″ is linear alkyl or alkenyl. In certain embodiments, M″ is branched, e.g., —CH(CH₃)CH₂—.

In some embodiments, each R⁵ is H. In some embodiments, each R⁶ is H. In certain such embodiments, each R⁵ and each R⁶ is H.

In some embodiments, R⁷ is H. In other embodiments, R⁷ is C₁-3 alkyl (e.g., methyl, ethyl, propyl, or i-propyl).

In some embodiments, R² and R³ are independently C₅₋₁₄ alkyl or C₅₋₁₄ alkenyl.

In some embodiments, R² and R³ are the same. In some embodiments, R² and R³ are C₈ alkyl. In certain embodiments, R² and R³ are C₂ alkyl. In other embodiments, R² and R³ are C₃ alkyl. In some embodiments, R² and R³ are C₄ alkyl. In certain embodiments, R² and R³ are C₅ alkyl. In other embodiments, R² and R³ are C₆ alkyl. In some embodiments, R² and R³ are C₇ alkyl.

In other embodiments, R² and R³ are different. In certain embodiments, R² is C₈ alkyl. In some embodiments, R³ is C₁₋₇ (e.g., C₁, C₂, C₃, C₄, C₅, C₆, or C₇ alkyl) or C₉ alkyl.

In some embodiments, R³ is C₁ alkyl. In some embodiments, R³ is C₂ alkyl. In some embodiments, R³ is C₃ alkyl. In some embodiments, R³ is C₄ alkyl. In some embodiments, R³ is C₅ alkyl. In some embodiments, R³ is C₆ alkyl. In some embodiments, R³ is C₇ alkyl. In some embodiments, R³ is C₉ alkyl.

In some embodiments, R⁷ and R³ are H.

In certain embodiments, R² is H.

In some embodiments, m is 5, 6, 7, 8, or 9. In some embodiments, m is 5, 7, or 9. For example, in some embodiments, m is 5. For example, in some embodiments, m is 7. For example, in some embodiments, m is 9.

In some embodiments, R⁴ is selected from —(CH₂)ₙQ and —(CH₂)ₙCHQR.

In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R,

—N(H)S(O)₂R, —N(R)C(O)N(R)₂, —N(H)C(O)N(R)₂, —N(H)C(O)N(H)(R), —N(R)C(S)N(R)₂,

—N(H)C(S)N(R)₂, —N(H)C(S)N(H)(R), —C(R)N(R)₂C(O)OR, —N(R)S(O)₂R⁸, a carbocycle, and a heterocycle.

In certain embodiments, Q is —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, or —N(R)C(O)OR.

In certain embodiments, Q is —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, or —N(OR)C(═CHR⁹)N(R)₂.

In certain embodiments, Q is thiourea or an isostere thereof, e.g.,

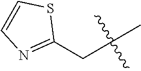 [SMILES: *Cc1nccs1]

or —NHC(═NR⁹)N(R)₂.

In certain embodiments, Q is —C(═NR⁹)N(R)₂. For example, when Q is —C(═NR⁹)N(R)₂, n is 4 or 5. For example, R⁹ is —S(O)₂N(R)₂.

In certain embodiments, Q is —C(═NR⁹)R or —C(O)N(R)OR, e.g., —CH(═N—OCH₃), —C(O)NH—OH, —C(O)NH—OCH₃, —C(O)N(CH₃)—OH, or —C(O)N(CH₃)—OCH₃.

In certain embodiments, Q is —OH.

In certain embodiments, Q is a substituted or unsubstituted 5- to 10-membered heteroaryl, e.g., Q is a triazole, an imidazole, a pyrimidine, a purine, 2-amino-1,9-dihydro-6H-purin-6-one-9-yl (or guanin-9-yl), adenin-9-yl, cytosin-1-yl, or uracil-1-yl, each of which is optionally substituted with one or more substituents selected from alkyl, OH, alkoxy, -alkyl-OH, -alkyl-O-alkyl, and the substituent can be further substituted. In certain embodiments, Q is a substituted 5- to 14-membered heterocycloalkyl, e.g., substituted with one or more substituents selected from oxo (═O), OH, amino, mono- or di-alkylamino, and C₁₋₃ alkyl. For example, Q is 4-methylpiperazinyl, 4-(4-methoxybenzyl)piperazinyl, isoindolin-2-yl-1,3-dione, pyrrolidin-1-yl-2,5-dione, or imidazolidin-3-yl-2,4-dione.

In certain embodiments, Q is —NHR⁸, in which R⁸ is a C₃₋₆ cycloalkyl optionally substituted with one or more substituents selected from oxo (═O), amino (NH₂), mono- or di-alkylamino, C₁₋₃ alkyl and halo. For example, R⁸ is cyclobutenyl, e.g., 3-(dimethylamino)-cyclobut-3-ene-4-yl-1,2-dione. In further embodiments, R⁸ is a C₃₋₆ cycloalkyl optionally substituted with one or more substituents selected from oxo (═O), thio (═S), amino (NH₂), mono- or di-alkylamino, C₁₋₃ alkyl, heterocycloalkyl, and halo, wherein the mono- or di-alkylamino, C₁₋₃ alkyl, and heterocycloalkyl are further substituted. For example R⁸ is cyclobutenyl substituted with one or more of oxo, amino, and alkylamino, wherein the alkylamino is further substituted, e.g., with one or more of C₁₋₃ alkoxy, amino, mono- or di-alkylamino, and halo. For example, R⁸ is 3-(((dimethylamino)ethyl)amino)cyclobut-3-enyl-1,2-dione. For example R⁸ is cyclobutenyl substituted with one or more of oxo, and alkylamino. For example, R⁸ is 3-(ethylamino)cyclobut-3-ene-1,2-dione. For example R⁸ is cyclobutenyl substituted with one or more of oxo, thio, and alkylamino. For example R⁸ is 3-(ethylamino)-4-thioxocyclobut-2-en-1-one or 2-(ethylamino)-4-thioxocyclobut-2-en-1-one. For example R⁸ is cyclobutenyl substituted with one or more of thio, and alkylamino. For example R⁸ is 3-(ethylamino)cyclobut-3-ene-1,2-dithione. For example R⁸ is cyclobutenyl substituted with one or more of oxo and dialkylamino. For example R⁸ is 3-(diethylamino)cyclobut-3-ene-1,2-dione. For example, R⁸ is cyclobutenyl substituted with one or more of oxo, thio, and dialkylamino. For example, R⁸ is 2-(diethylamino)-4-thioxocyclobut-2-en-1-one or 3-(diethylamino)-4-thioxocyclobut-2-en-1-one. For example, R⁸ is cyclobutenyl substituted with one or more of thio, and dialkylamino. For example, R⁸ is 3-(diethylamino)cyclobut-3-ene-1,2-dithione. For example, R⁸ is cyclobutenyl substituted with one or more of oxo and alkylamino or dialkylamino, wherein alkylamino or dialkylamino is further substituted, e.g. with one or more alkoxy. For example, R⁸ is 3-(bis(2-methoxyethyl)amino)cyclobut-3-ene-1,2-dione. For example, R⁸ is cyclobutenyl substituted with one or more of oxo, and heterocycloalkyl. For example, R⁸ is cyclobutenyl substituted with one or more of oxo, and piperidinyl, piperazinyl, or morpholinyl. For example, R⁸ is cyclobutenyl substituted with one or more of oxo, and heterocycloalkyl, wherein heterocycloalkyl is further substituted, e.g., with one or more C₁₋₃ alkyl. For example, R⁸ is cyclobutenyl substituted with one or more of oxo, and heterocycloalkyl, wherein heterocycloalkyl (e.g., piperidinyl, piperazinyl, or morpholinyl) is further substituted with methyl.

In certain embodiments, Q is —NHR⁸, in which R⁸ is a heteroaryl optionally substituted with one or more substituents selected from amino (NH₂), mono- or di-alkylamino, C₁₋₃ alkyl and halo. For example, R⁸ is thiazole or imidazole.

In certain embodiments, Q is —NHC(═NR⁹)N(R)₂ in which R⁹ is CN, C₁₋₆ alkyl, NO₂, —S(O)₂N(R)₂, —OR, —S(O)₂R, or H. For example, Q is —NHC(═NR⁹)N(CH₃)₂, —NHC(═NR⁹)NHCH₃, —NHC(═NR⁹)NH₂. In some embodiments, Q is —NHC(═NR⁹)N(R)₂ in which R⁹ is CN and R is C₁₋₃ alkyl substituted with mono- or di-alkylamino, e.g., R is ((dimethylamino)ethyl)amino. In some embodiments, Q is —NHC(═NR⁹)N(R)₂ in which R⁹ is C₁₋₆ alkyl, NO₂, —S(O)₂N(R)₂, —OR, —S(O)₂R, or H and R is C₁₋₃ alkyl substituted with mono- or di-alkylamino, e.g., R is ((dimethylamino)ethyl)amino.

In certain embodiments, Q is —NHC(═CHR⁹)N(R)₂, in which R⁹ is NO₂, CN, C₁₋₆ alkyl, —S(O)₂N(R)₂, —OR, —S(O)₂R, or H. For example, Q is —NHC(═CHR⁹)N(CH₃)₂, —NHC(═CHR⁹)NHCH₃, or —NHC(═CHR⁹)NH₂.

In certain embodiments, Q is —OC(O)N(R)₂, —N(R)C(O)OR, —N(OR)C(O)OR, such as —OC(O)NHCH₃, —N(OH)C(O)OCH₃, —N(OH)C(O)CH₃, —N(OCH₃)C(O)OCH₃, —N(OCH₃)C(O)CH₃, —N(OH)S(O)₂CH₃, or —NHC(O)OCH₃.

In certain embodiments, Q is —N(R)C(O)R, in which R is alkyl optionally substituted with C₁₋₃ alkoxyl or S(O)_{z}C₁₋₃ alkyl, in which z is 0, 1, or 2.

In certain embodiments, Q is an unsubstituted or substituted C₆₋₁₀ aryl (such as phenyl) or C₃₋₆ cycloalkyl.

In some embodiments, n is 1. In other embodiments, n is 2. In further embodiments, n is 3. In certain other embodiments, n is 4. For example, R⁴ may be —(CH₂)₂OH. For example, R⁴ may be —(CH₂)₃OH. For example, R⁴ may be —(CH₂)₄OH. For example, R⁴ may be benzyl. For example, R⁴ may be 4-methoxybenzyl.

In some embodiments, R⁴ is a C₃₋₆ carbocycle. In some embodiments, R⁴ is a C₃₋₆ cycloalkyl. For example, R⁴ may be cyclohexyl optionally substituted with e.g., OH, halo, C₁₋₆ alkyl, etc. For example, R⁴ may be 2-hydroxycyclohexyl.

In some embodiments, R is H.

In some embodiments, R is C₁₋₃ alkyl substituted with mono- or di-alkylamino, e.g., R is ((dimethylamino)ethyl)amino.

In some embodiments, R is C₁₋₆ alkyl substituted with one or more substituents selected from the group consisting of C₁₋₃ alkoxyl, amino, and C₁-C₃ dialkylamino.

In some embodiments, R is unsubstituted C₁₋₃ alkyl or unsubstituted C₂₋₃ alkenyl. For example, R⁴ may be —CH₂CH(OH)CH₃, —CH(CH₃)CH₂OH, or —CH₂CH(OH)CH₂CH₃.

In some embodiments, R is substituted C₁₋₃ alkyl, e.g., CH₂OH. For example, R⁴ may be —CH₂CH(OH)CH₂OH, —(CH₂)₃NHC(O)CH₂OH, —(CH₂)₃NHC(O)CH₂OBn, —(CH₂)₂O (CH₂)₂OH, —(CH₂)₃NHCH₂OCH₃, —(CH₂)₃NHCH₂OCH₂CH₃, CH₂SCH₃, CH₂S(O)CH₃, CH₂S(O)₂CH₃, or —CH(CH₂OH)₂.

In some embodiments, R⁴ is selected from any of the following groups:

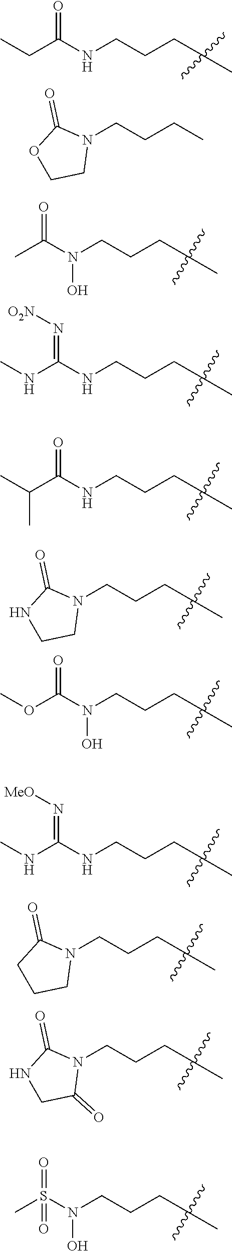

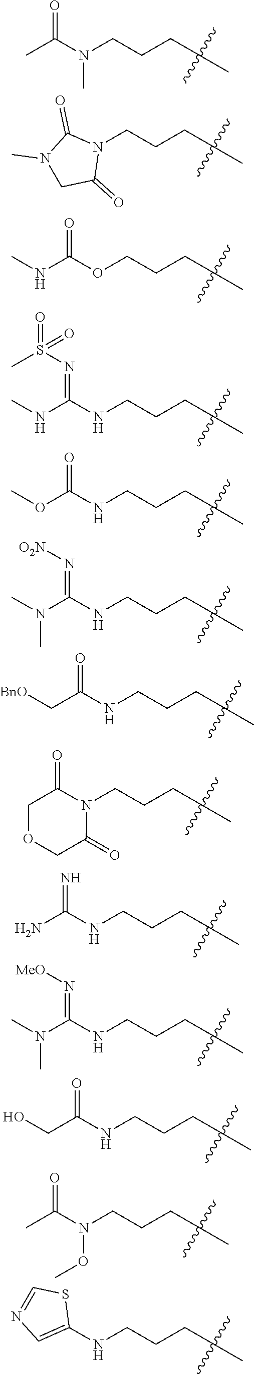

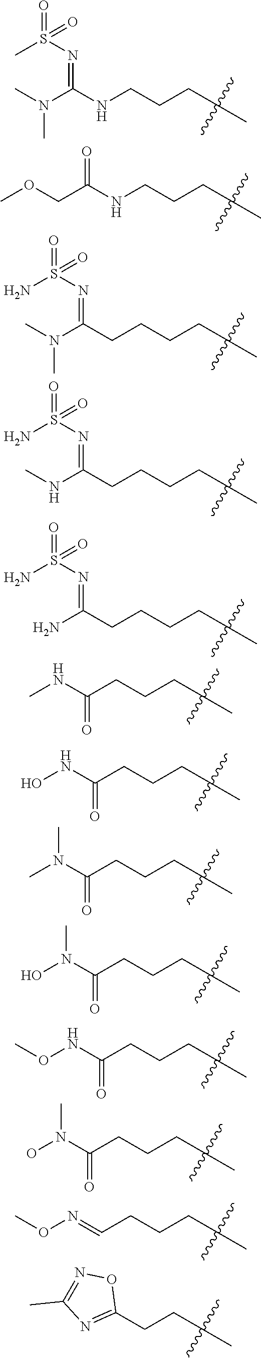

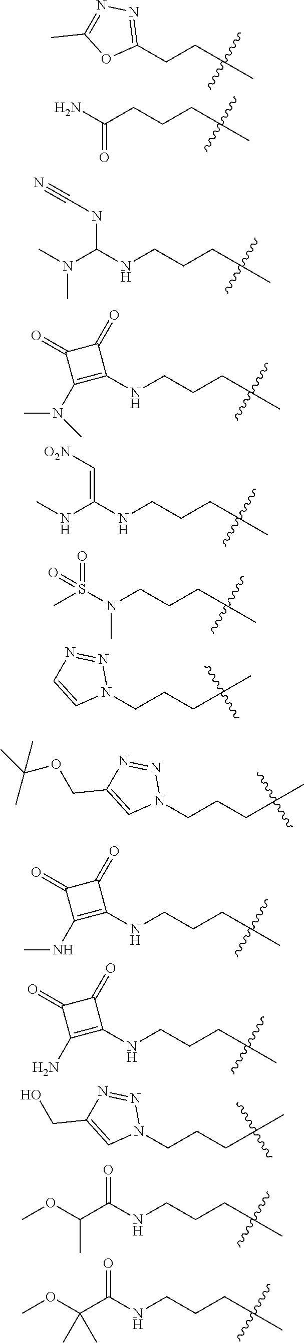

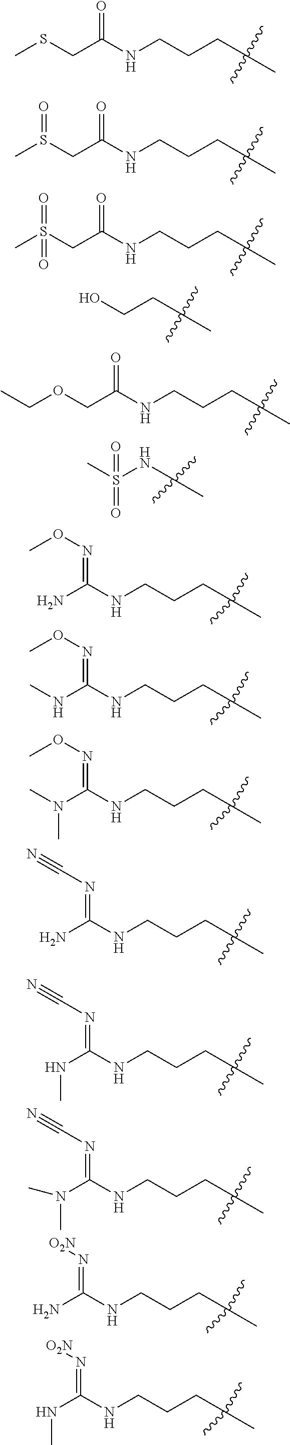

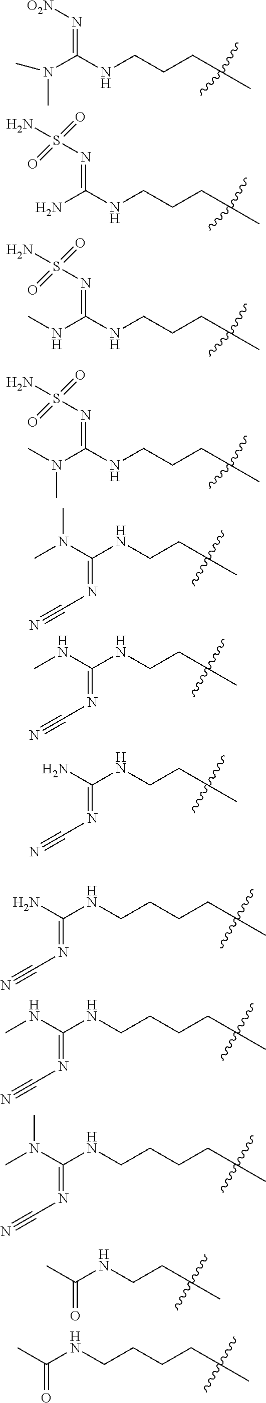

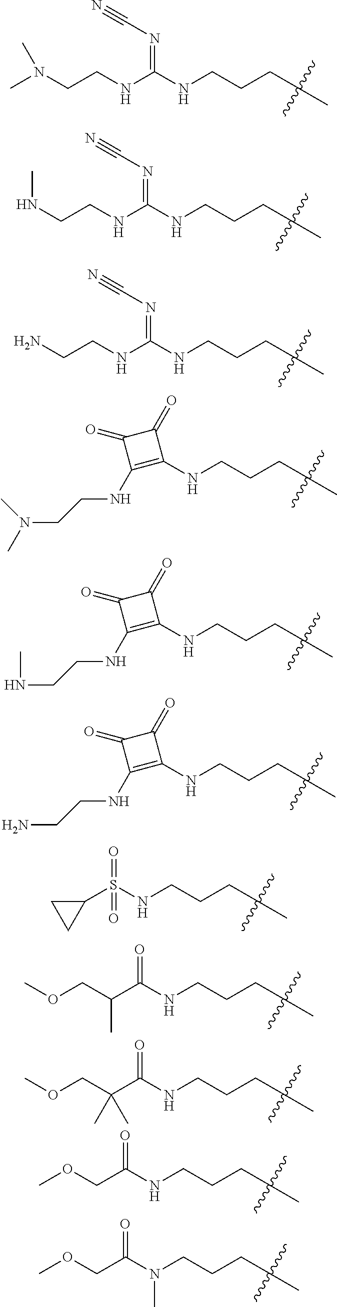

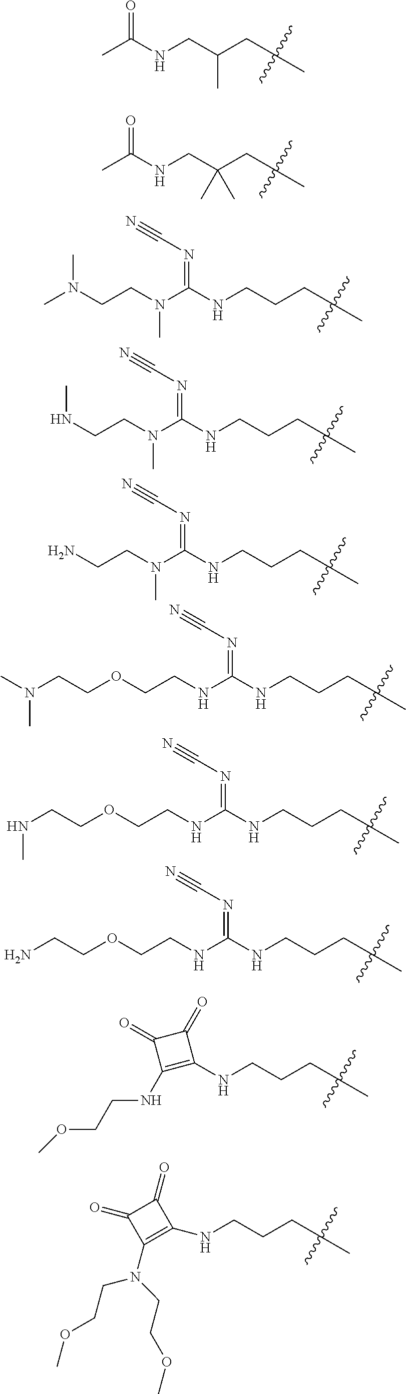

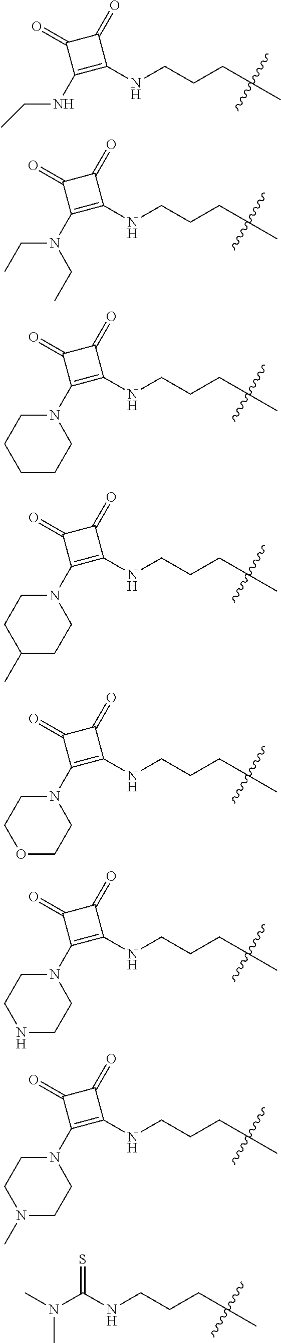

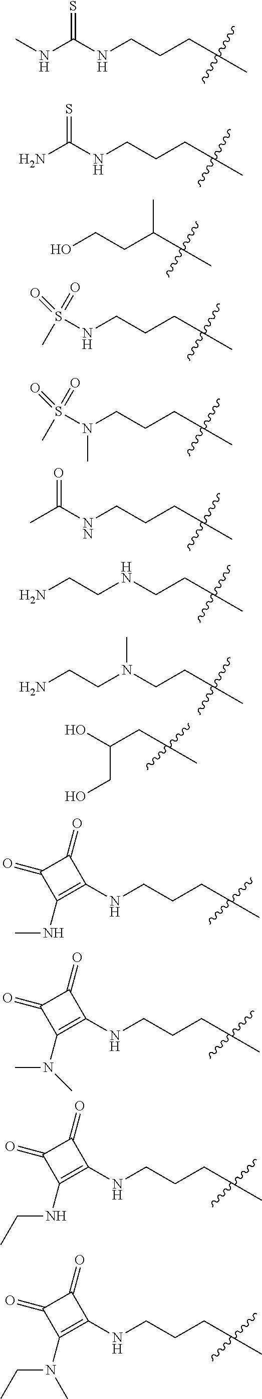

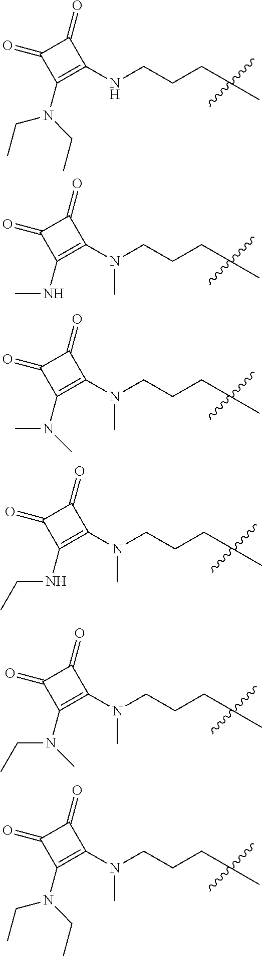

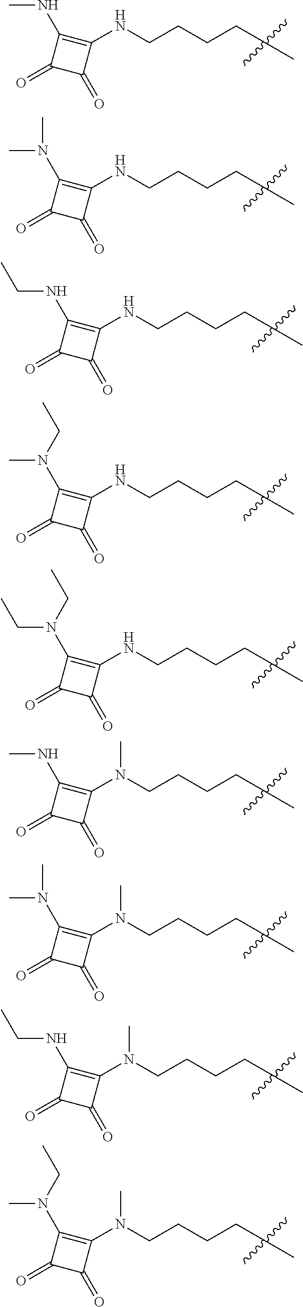

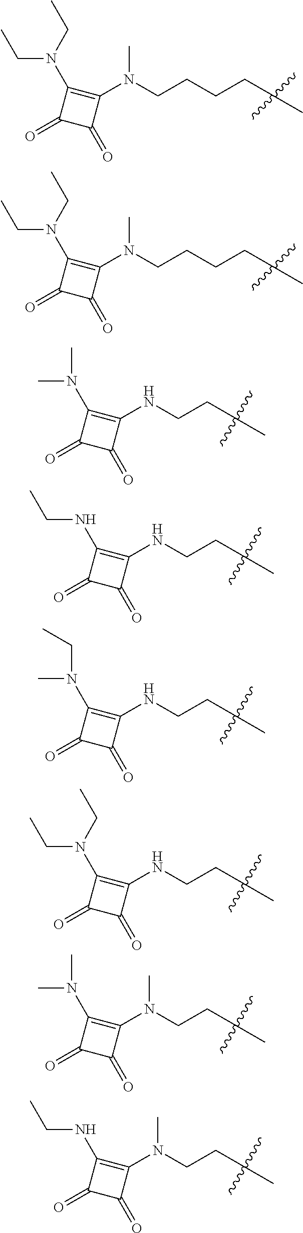

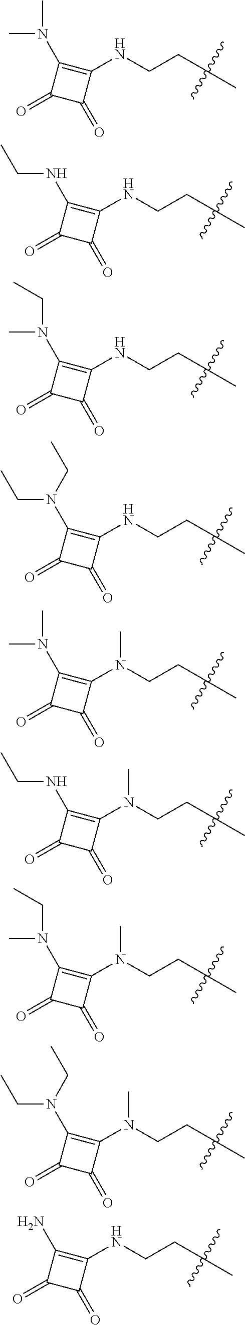

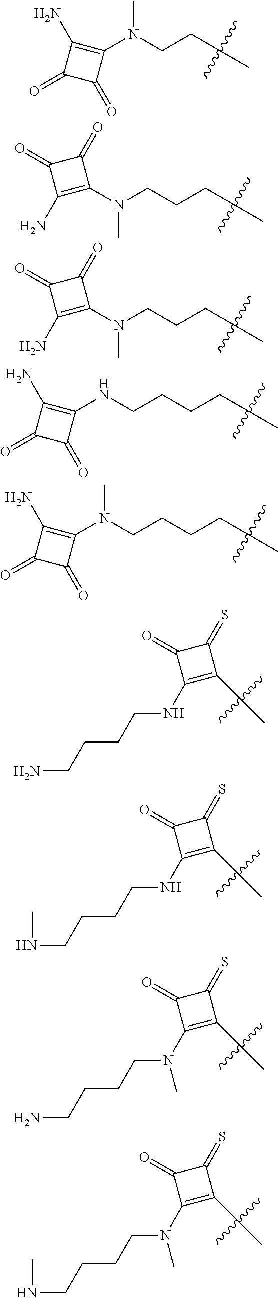

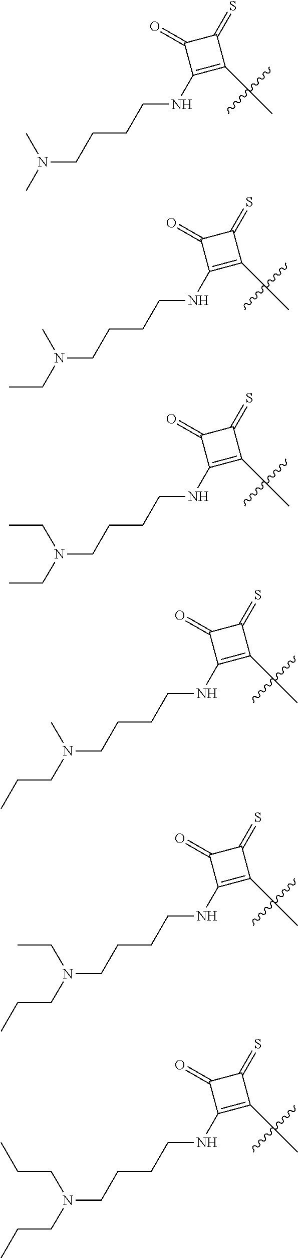 [SMILES: *c1c(NCCCCN(C)C)c(=O)c1=S ; *c1c(NCCCCN(C)CC)c(=O)c1=S ; *c1c(NCCCCN(CC)CC)c(=O)c1=S ; *c1c(NCCCCN(C)CCC)c(=O)c1=S ; *c1c(NCCCCN(CC)CCC)c(=O)c1=S ; *c1c(NCCCCN(CCC)CCC)c(=O)c1=S]

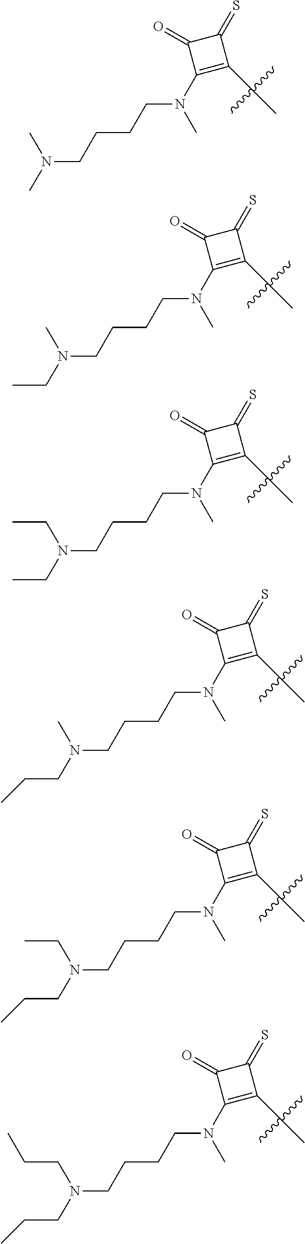 [SMILES: *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S ; *c1c(N(C)CCCCN(CC)CCC)c(=O)c1=S ; *c1c(N(C)CCCCN(CCC)CCC)c(=O)c1=S]

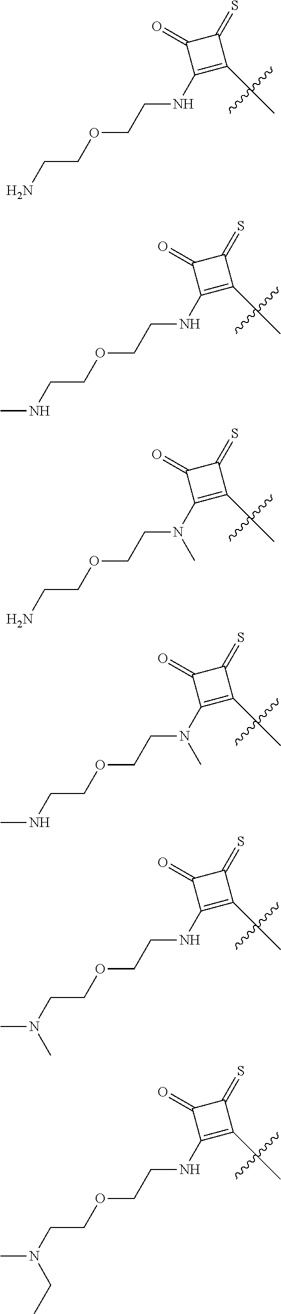 [SMILES: *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S ; *c1c(NCCOCCN(C)C)c(=O)c1=S ; *c1c(NCCOCCN(C)CC)c(=O)c1=S]

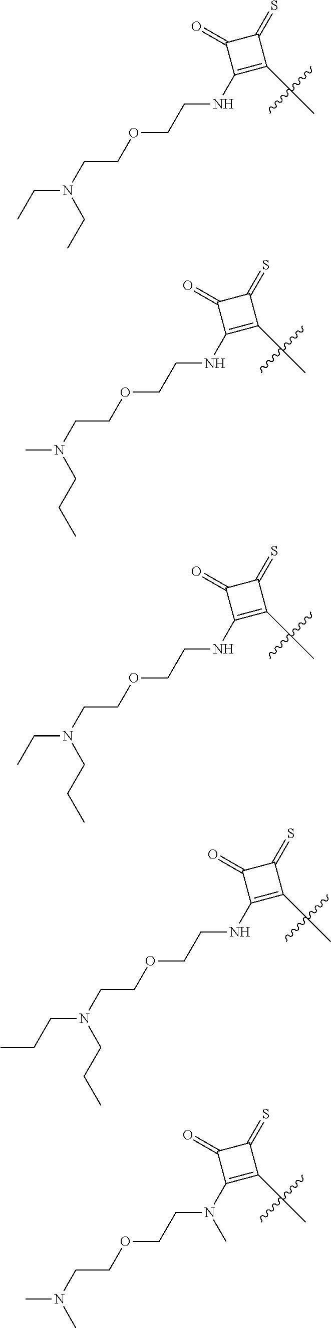 [SMILES: *c1c(NCCOCCN(CC)CC)c(=O)c1=S ; *c1c(NCCOCCN(C)CCC)c(=O)c1=S ; *c1c(NCCOCCN(CC)CCC)c(=O)c1=S ; *c1c(NCCOCCN(CCC)CCC)c(=O)c1=S ; *c1c(N(C)CCOCCN(C)C)c(=O)c1=S]

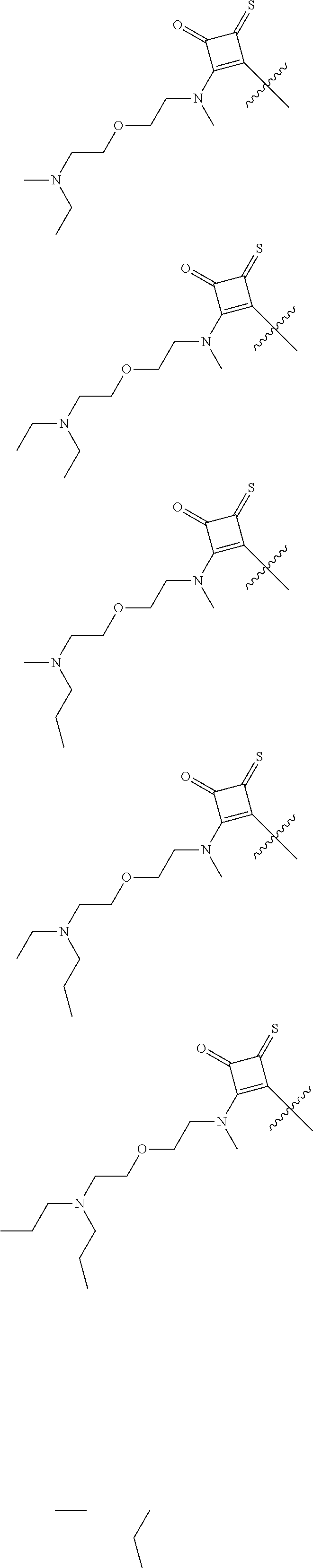

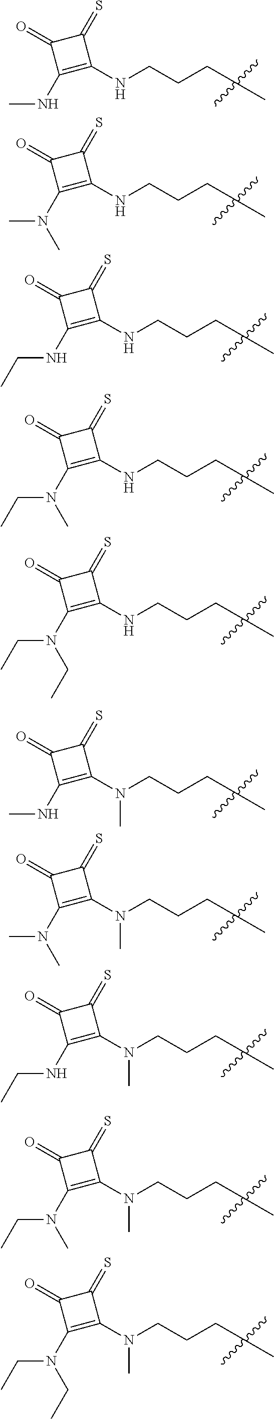

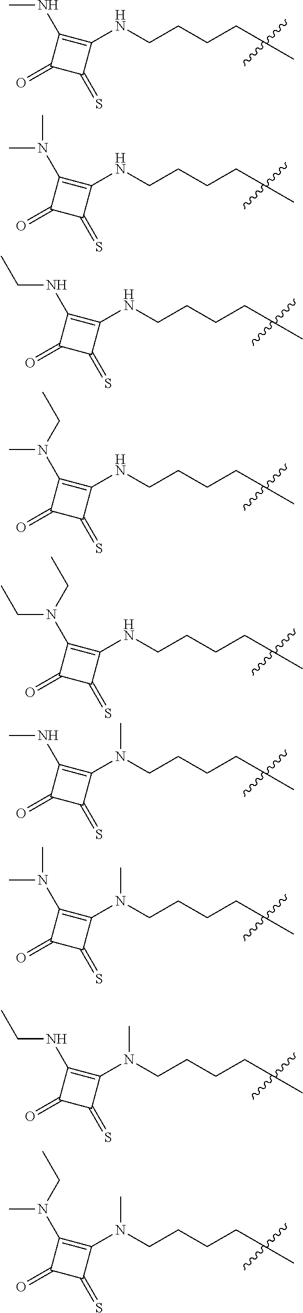

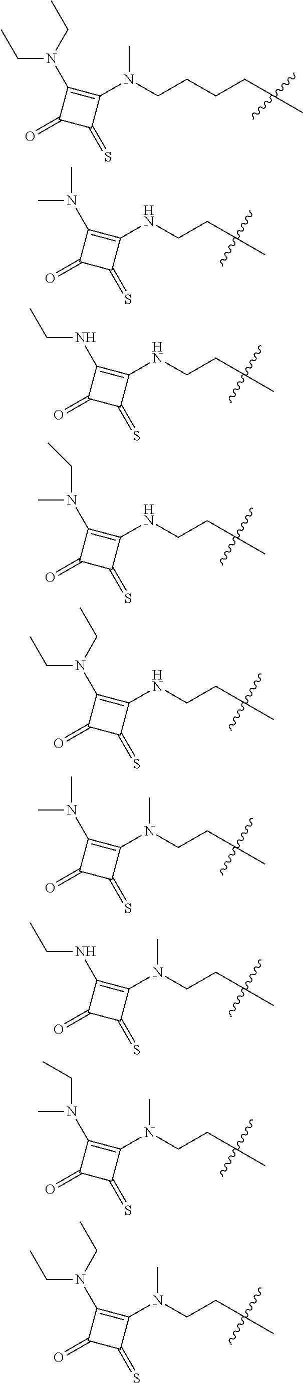

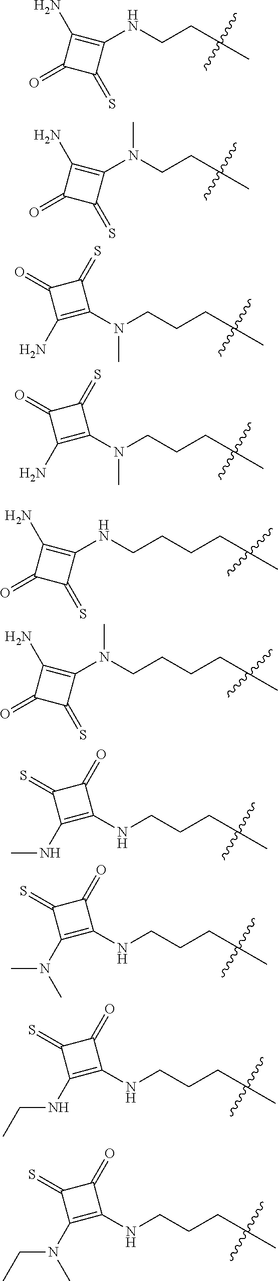

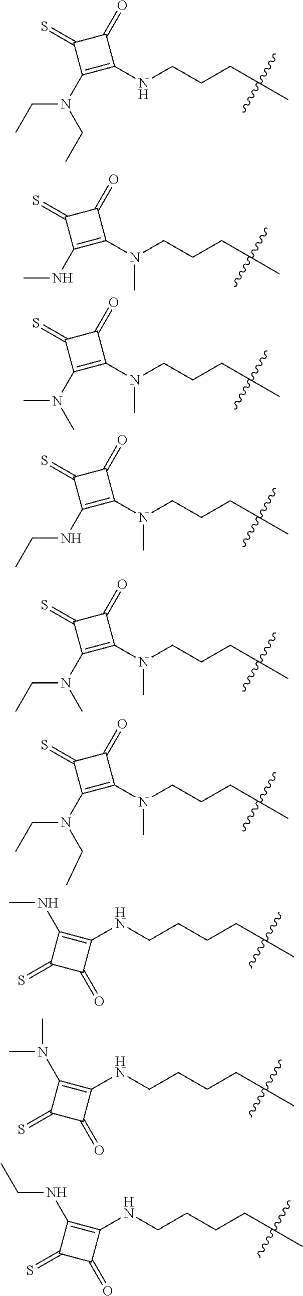

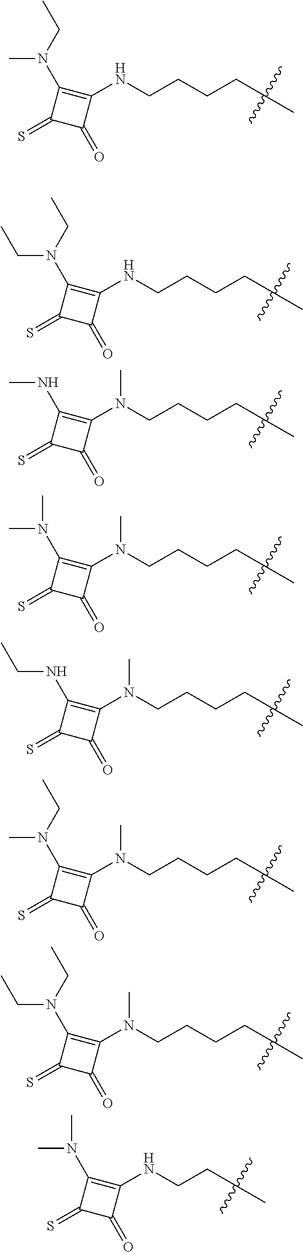

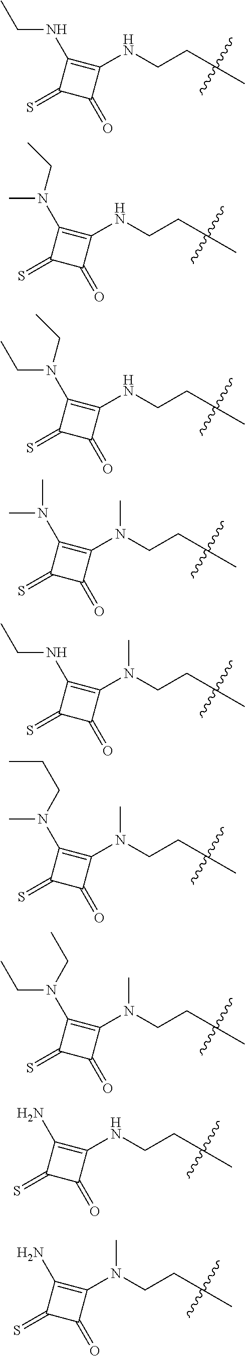

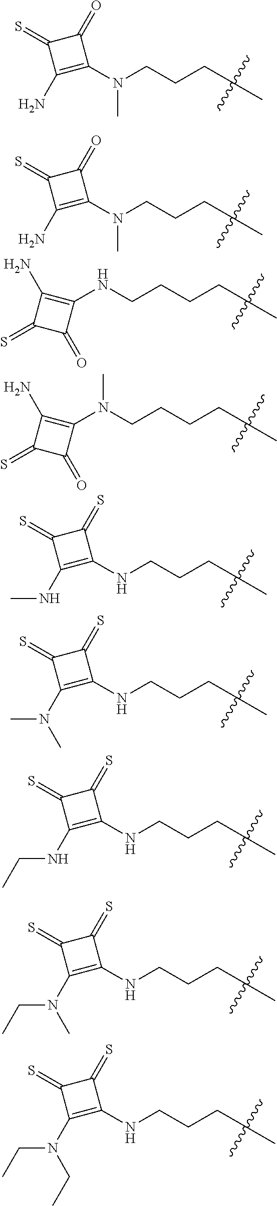

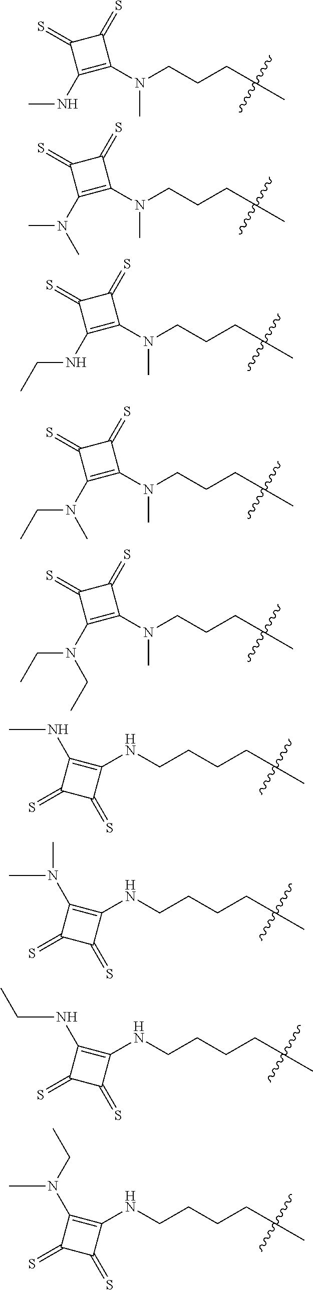

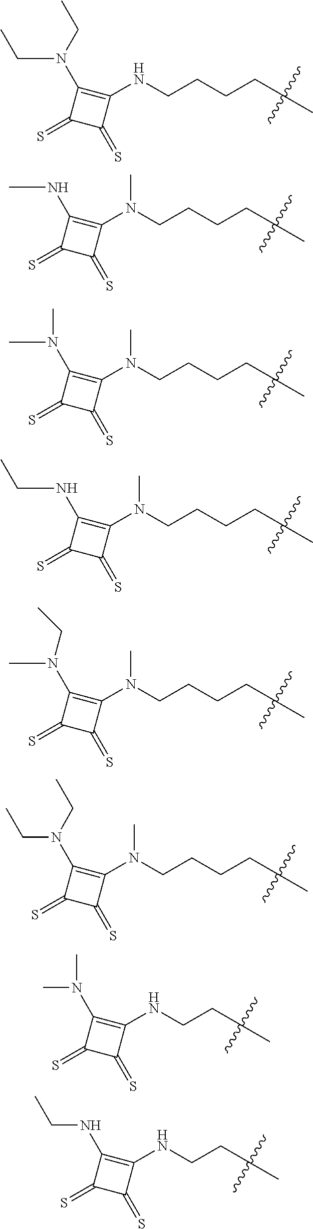

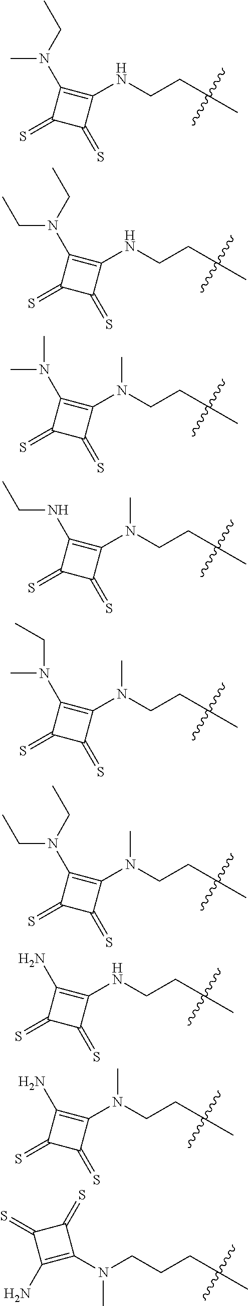

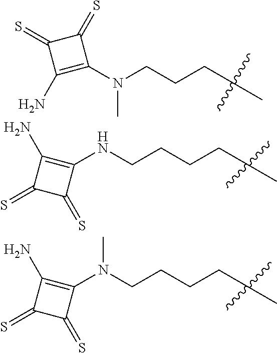

In some embodiments,

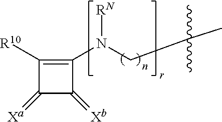

is selected from any of the following groups:

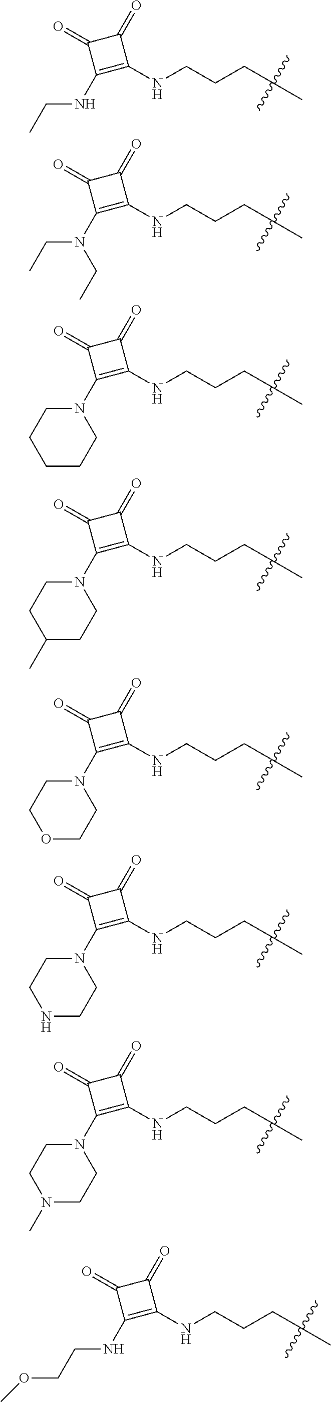

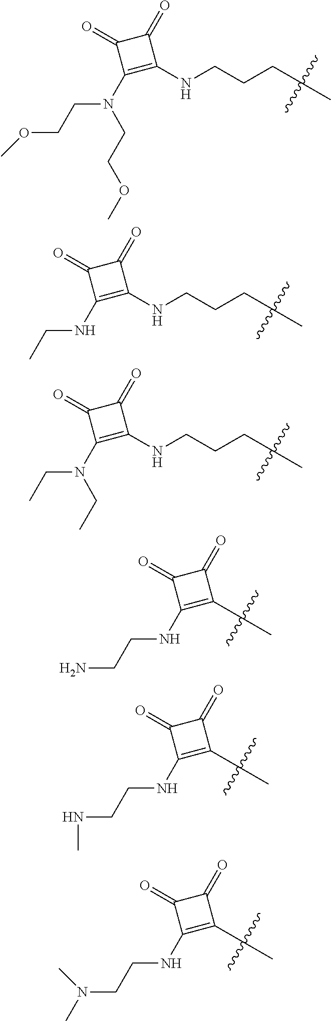

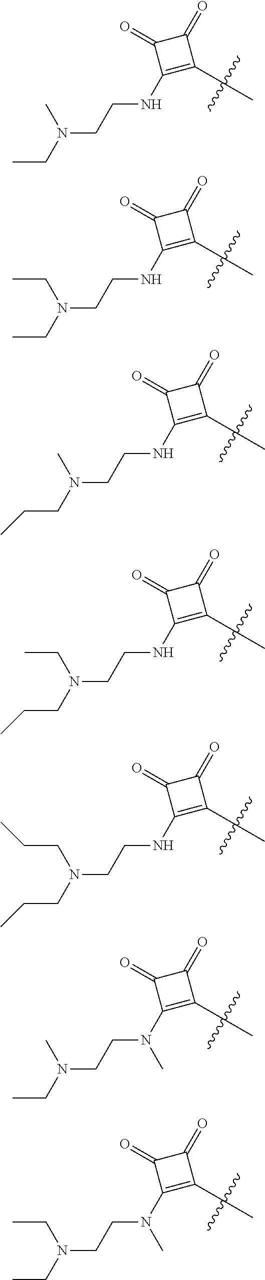

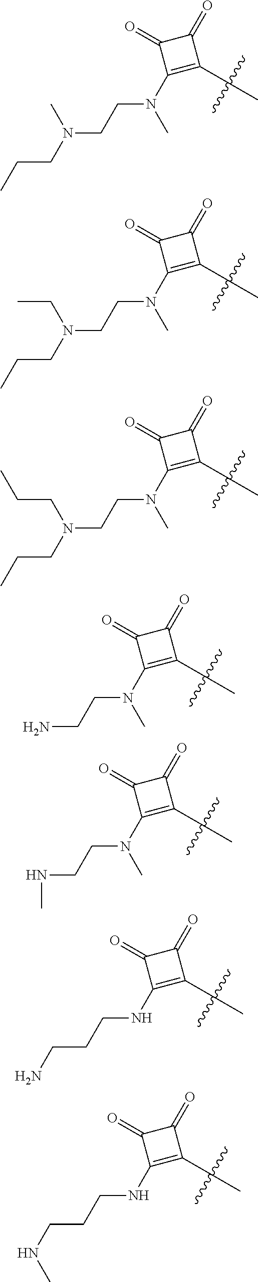

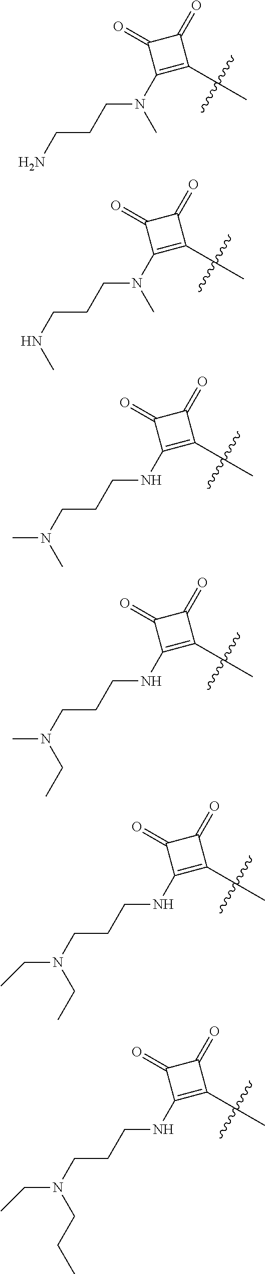 [SMILES: *c1c(N(C)CCCN)c(=O)c1=O ; *c1c(N(C)CCCNC)c(=O)c1=O ; *c1c(NCCCN(C)C)c(=O)c1=O ; *c1c(NCCCN(C)CC)c(=O)c1=O ; *c1c(NCCCN(CC)CC)c(=O)c1=O ; *c1c(NCCCN(CC)CCC)c(=O)c1=O]

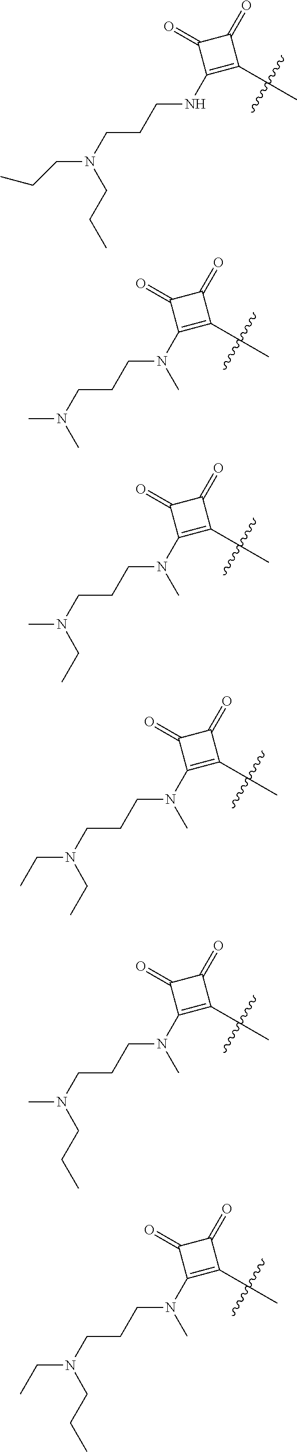 [SMILES: *c1c(NCCCN(CCC)CCC)c(=O)c1=O ; *c1c(N(C)CCCN(C)C)c(=O)c1=O ; *c1c(N(C)CCCN(C)CC)c(=O)c1=O ; *c1c(N(C)CCCN(CC)CC)c(=O)c1=O ; *c1c(N(C)CCCN(C)CCC)c(=O)c1=O ; *c1c(N(C)CCCN(CC)CCC)c(=O)c1=O]

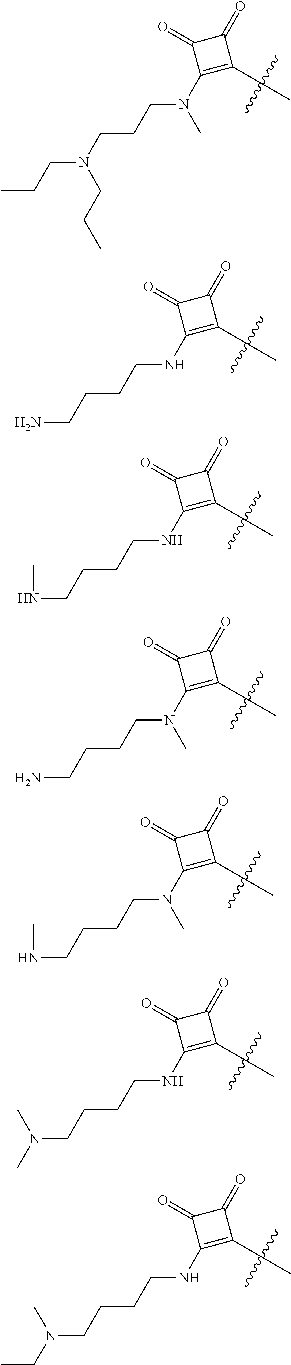

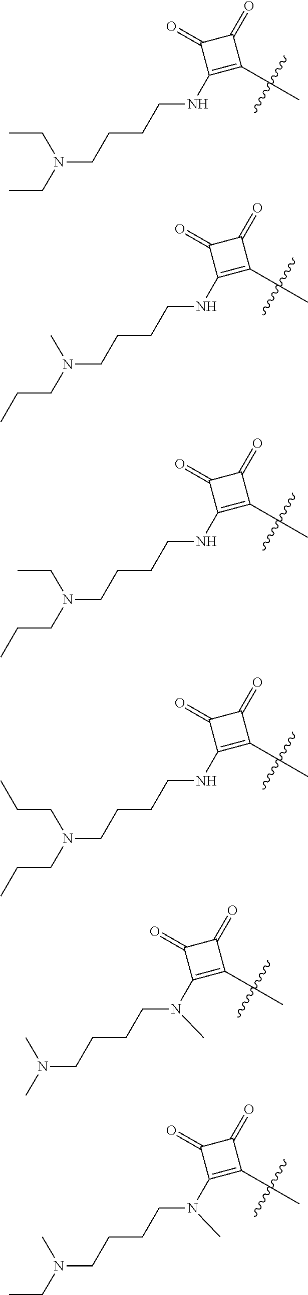 [SMILES: *c1c(NCCCCN(CC)CC)c(=O)c1=O ; *c1c(NCCCCN(C)CCC)c(=O)c1=O ; *c1c(NCCCCN(CC)CCC)c(=O)c1=O ; *c1c(NCCCCN(CCC)CCC)c(=O)c1=O ; *c1c(N(C)CCCCN(C)C)c(=O)c1=O ; *c1c(N(C)CCCCN(C)CC)c(=O)c1=O]

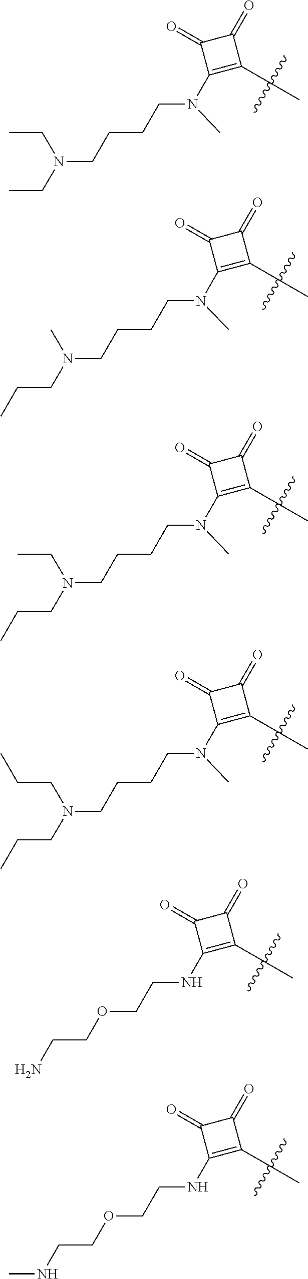 [SMILES: *c1c(N(C)CCCCN(CC)CC)c(=O)c1=O ; *c1c(N(C)CCCCN(C)CCC)c(=O)c1=O ; *c1c(N(C)CCCCN(CC)CCC)c(=O)c1=O ; *c1c(N(C)CCCCN(CCC)CCC)c(=O)c1=O ; *c1c(NCCOCCN)c(=O)c1=O ; *c1c(NCCOCCNC)c(=O)c1=O]

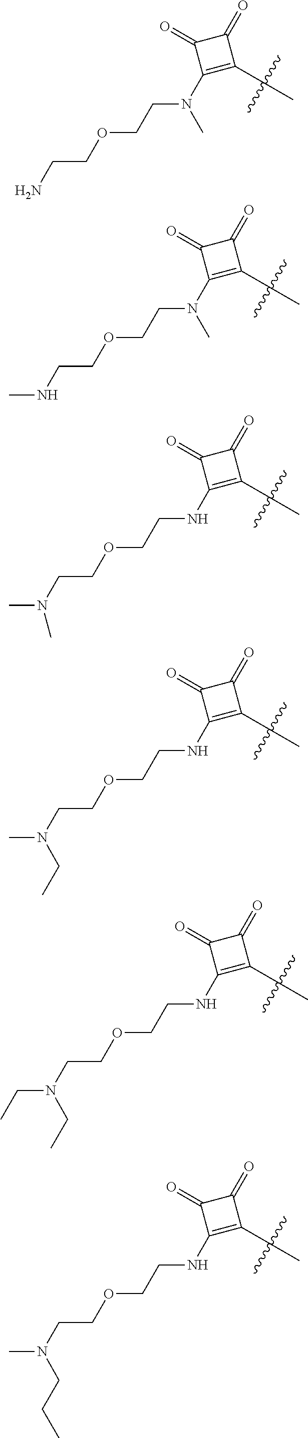 [SMILES: *c1c(N(C)CCOCCN)c(=O)c1=O ; *c1c(N(C)CCOCCNC)c(=O)c1=O ; *c1c(NCCOCCN(C)C)c(=O)c1=O ; *c1c(NCCOCCN(C)CC)c(=O)c1=O ; *c1c(NCCOCCN(CC)CC)c(=O)c1=O ; *c1c(NCCOCCN(C)CCC)c(=O)c1=O]

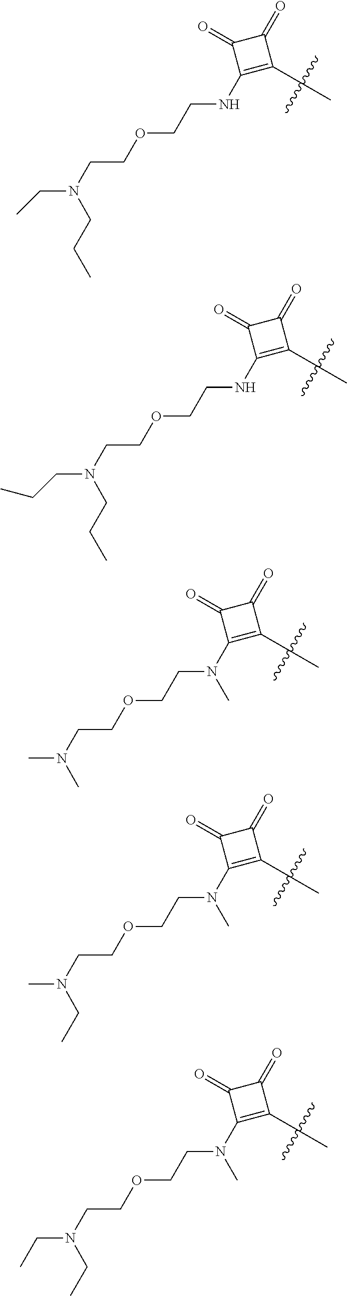 [SMILES: *c1c(NCCOCCN(CC)CCC)c(=O)c1=O ; *c1c(NCCOCCN(CCC)CCC)c(=O)c1=O ; *c1c(N(C)CCOCCN(C)C)c(=O)c1=O ; *c1c(N(C)CCOCCN(C)CC)c(=O)c1=O ; *c1c(N(C)CCOCCN(CC)CC)c(=O)c1=O]

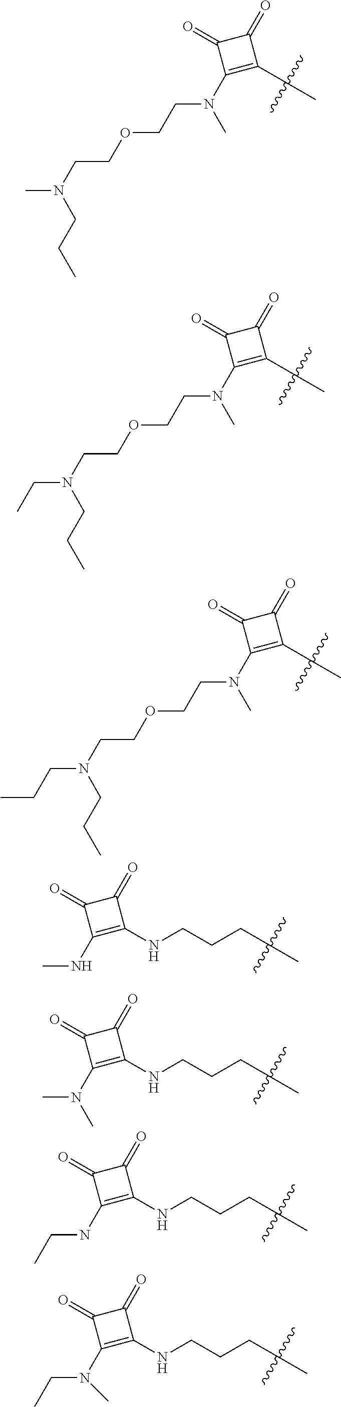

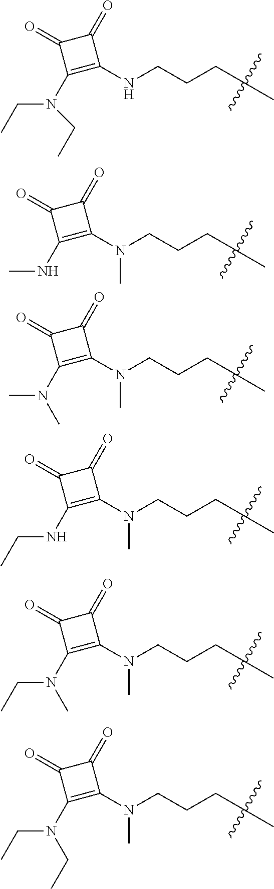

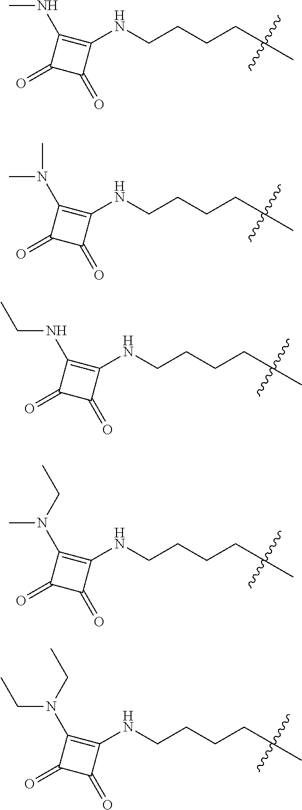 [SMILES: *CCCCNc1c(NC)c(=O)c1=O ; *CCCCNc1c(N(C)C)c(=O)c1=O ; *CCCCNc1c(NCC)c(=O)c1=O ; *CCCCNc1c(N(C)CC)c(=O)c1=O ; *CCCCNc1c(N(CC)CC)c(=O)c1=O]

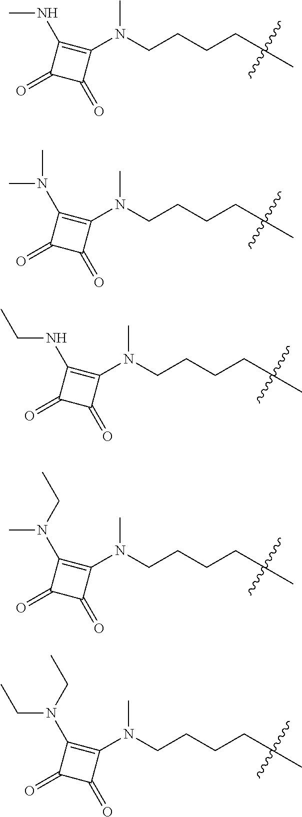 [SMILES: *CCCCN(C)c1c(NC)c(=O)c1=O ; *CCCCN(C)c1c(N(C)C)c(=O)c1=O ; *CCCCN(C)c1c(NCC)c(=O)c1=O ; *CCCCN(C)c1c(N(C)CC)c(=O)c1=O ; *CCCCN(C)c1c(N(CC)CC)c(=O)c1=O]

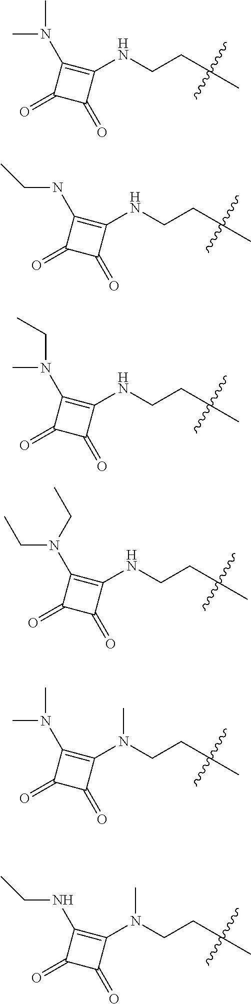 [SMILES: *CCNc1c(N(C)C)c(=O)c1=O ; *CCNc1c(NCC)c(=O)c1=O ; *CCNc1c(N(C)CC)c(=O)c1=O ; *CCNc1c(N(CC)CC)c(=O)c1=O ; *CCN(C)c1c(N(C)C)c(=O)c1=O ; *CCN(C)c1c(NCC)c(=O)c1=O]

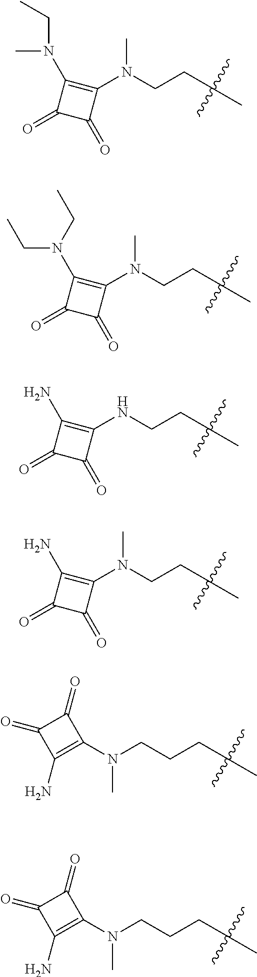

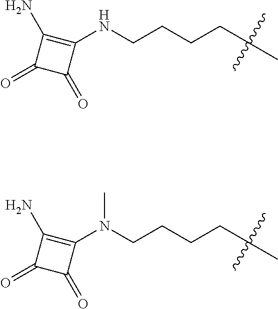 [SMILES: *CCCCNc1c(N)c(=O)c1=O ; *CCCCN(C)c1c(N)c(=O)c1=O]

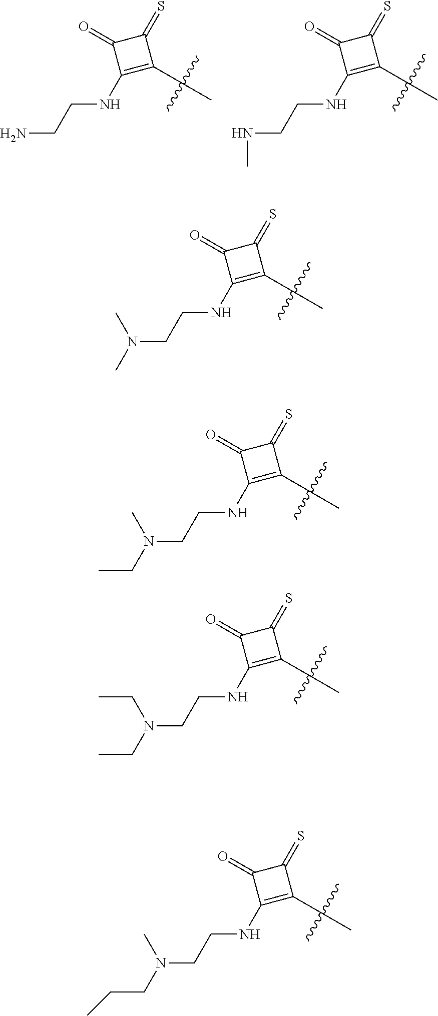 [SMILES: *c1c(NCCN)c(=O)c1=S ; *c1c(NCCNC)c(=O)c1=S ; *c1c(NCCN(C)C)c(=O)c1=S ; *c1c(NCCN(C)CC)c(=O)c1=S ; *c1c(NCCN(CC)CC)c(=O)c1=S ; *c1c(NCCN(C)CCC)c(=O)c1=S]

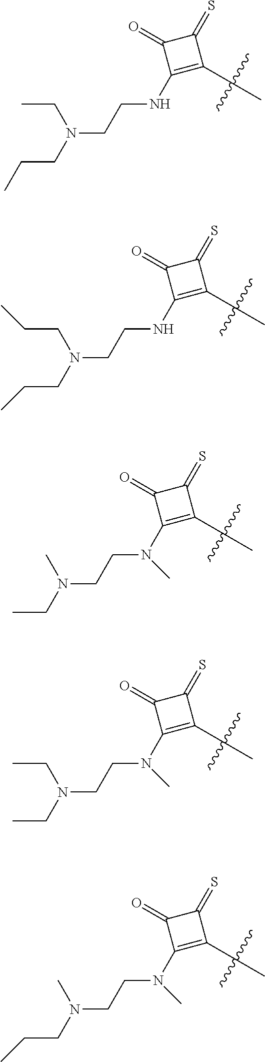 [SMILES: *c1c(NCCN(CC)CCC)c(=O)c1=S ; *c1c(NCCN(CCC)CCC)c(=O)c1=S ; *c1c(N(C)CCN(C)CC)c(=O)c1=S ; *c1c(N(C)CCN(CC)CC)c(=O)c1=S ; *c1c(N(C)CCN(C)CCC)c(=O)c1=S]

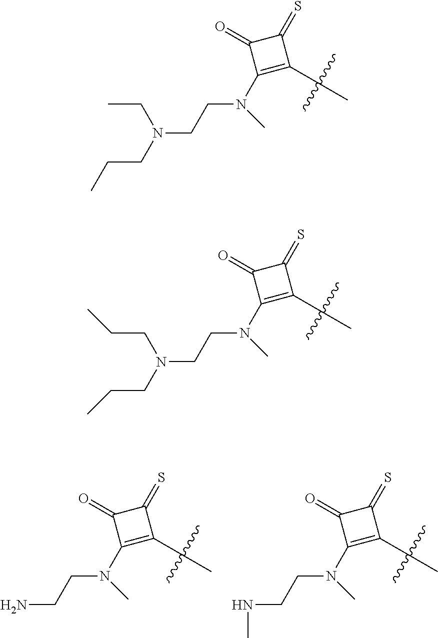 [SMILES: *c1c(N(C)CCN(CC)CCC)c(=O)c1=S ; *c1c(N(C)CCN(CCC)CCC)c(=O)c1=S ; *c1c(N(C)CCN)c(=O)c1=S ; *c1c(N(C)CCNC)c(=O)c1=S]

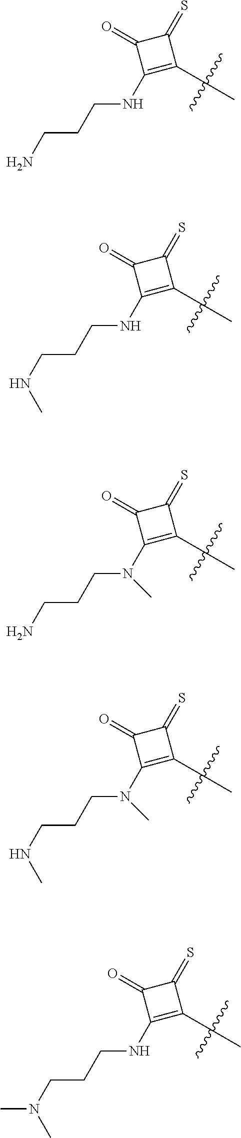 [SMILES: *c1c(NCCCN)c(=O)c1=S ; *c1c(NCCCNC)c(=O)c1=S ; *c1c(N(C)CCCN)c(=O)c1=S ; *c1c(N(C)CCCNC)c(=O)c1=S ; *c1c(NCCCN(C)C)c(=O)c1=S]

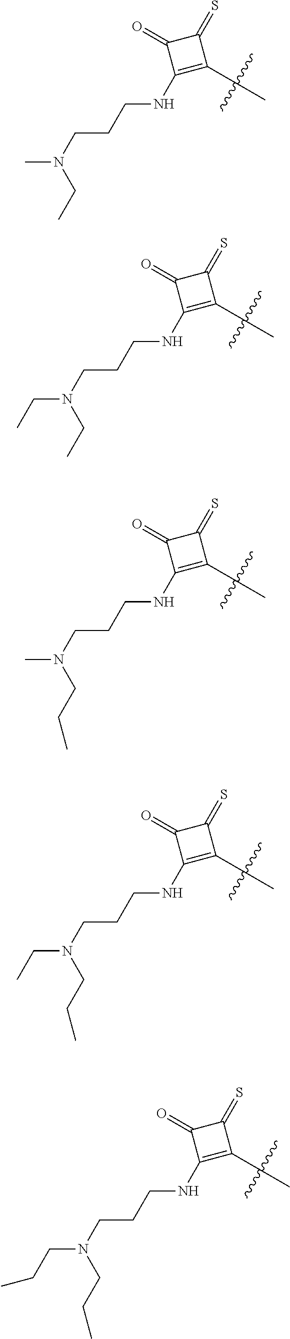 [SMILES: *c1c(NCCCN(C)CC)c(=O)c1=S ; *c1c(NCCCN(CC)CC)c(=O)c1=S ; *c1c(NCCCN(C)CCC)c(=O)c1=S ; *c1c(NCCCN(CC)CCC)c(=O)c1=S ; *c1c(NCCCN(CCC)CCC)c(=O)c1=S]

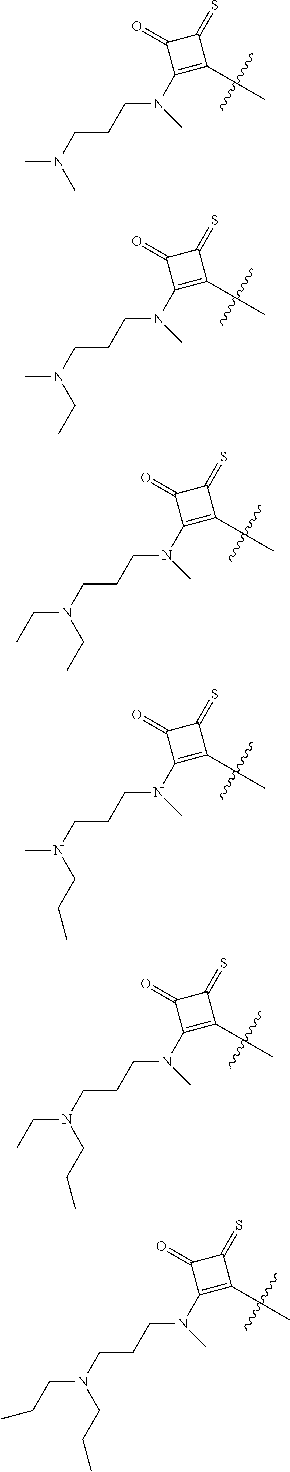 [SMILES: *c1c(N(C)CCCN(C)C)c(=O)c1=S ; *c1c(N(C)CCCN(C)CC)c(=O)c1=S ; *c1c(N(C)CCCN(CC)CC)c(=O)c1=S ; *c1c(N(C)CCCN(C)CCC)c(=O)c1=S ; *c1c(N(C)CCCN(CC)CCC)c(=O)c1=S ; *c1c(N(C)CCCN(CCC)CCC)c(=O)c1=S]

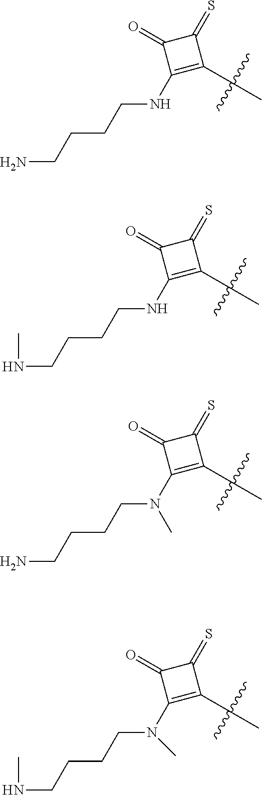 [SMILES: *c1c(NCCCCN)c(=O)c1=S ; *c1c(NCCCCNC)c(=O)c1=S ; *c1c(N(C)CCCCN)c(=O)c1=S ; *c1c(N(C)CCCCNC)c(=O)c1=S]

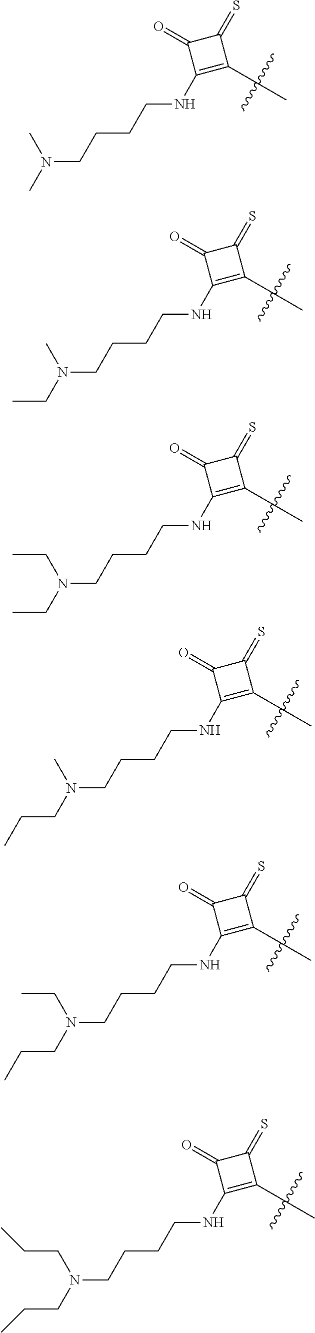 [SMILES: *c1c(NCCCCN(C)C)c(=O)c1=S ; *c1c(NCCCCN(C)CC)c(=O)c1=S ; *c1c(NCCCCN(CC)CC)c(=O)c1=S ; *c1c(NCCCCN(C)CCC)c(=O)c1=S ; *c1c(NCCCCN(CC)CCC)c(=O)c1=S ; *c1c(NCCCCN(CCC)CCC)c(=O)c1=S]

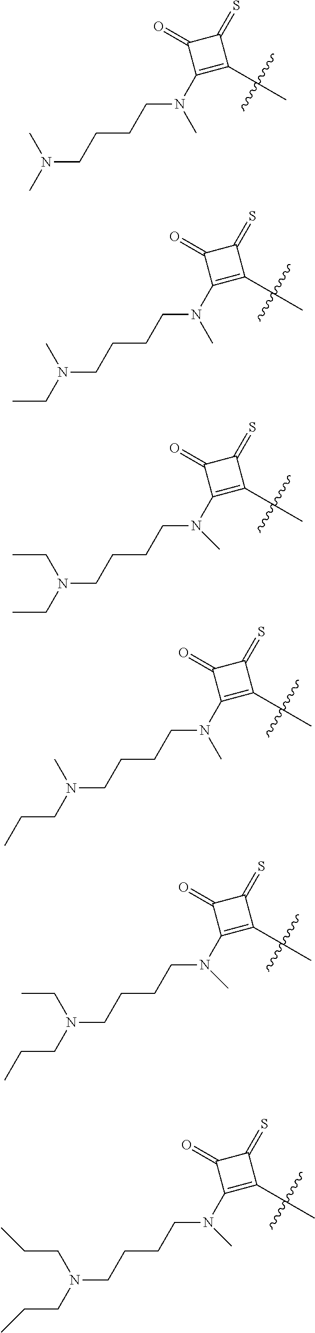 [SMILES: *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S ; *c1c(N(C)CCCCN(CC)CCC)c(=O)c1=S ; *c1c(N(C)CCCCN(CCC)CCC)c(=O)c1=S]

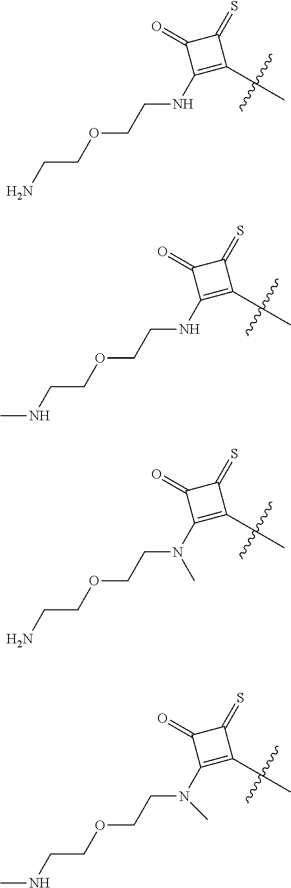 [SMILES: *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S]

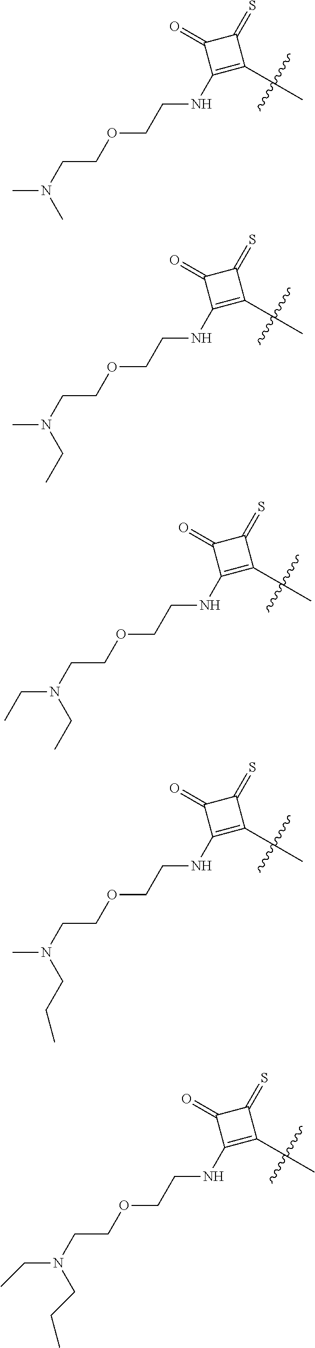 [SMILES: *c1c(NCCOCCN(C)C)c(=O)c1=S ; *c1c(NCCOCCN(C)CC)c(=O)c1=S ; *c1c(NCCOCCN(CC)CC)c(=O)c1=S ; *c1c(NCCOCCN(C)CCC)c(=O)c1=S ; *c1c(NCCOCCN(CC)CCC)c(=O)c1=S]

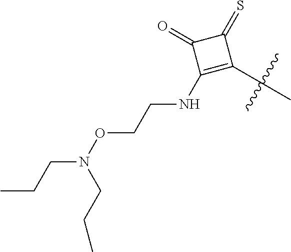 [SMILES: *c1c(NCCON(CCC)CCC)c(=O)c1=S]

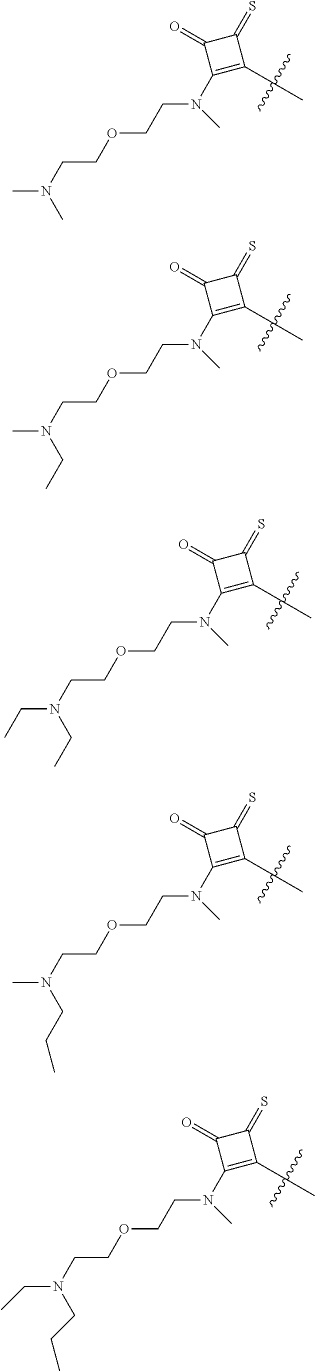 [SMILES: *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S ; *c1c(N(C)CCOCCN(CC)CCC)c(=O)c1=S]

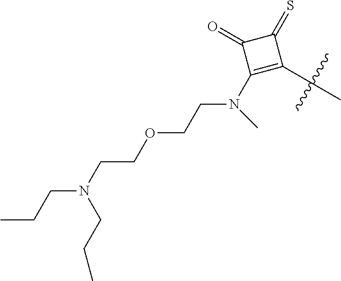 [SMILES: *c1c(N(C)CCOCCN(CCC)CCC)c(=O)c1=S]

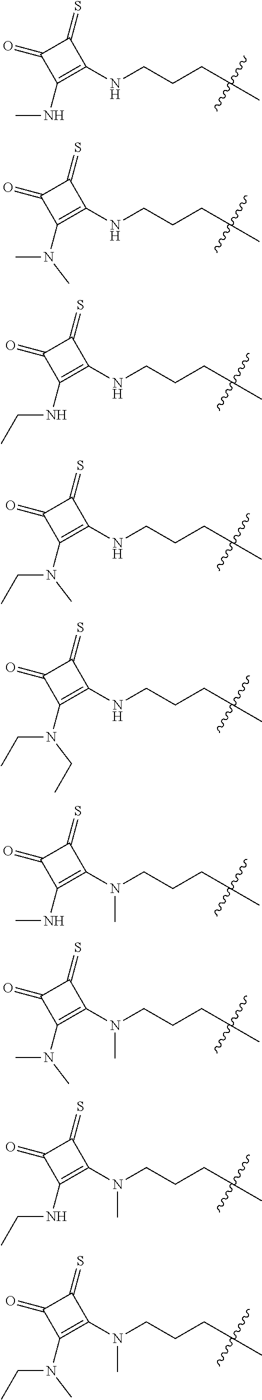

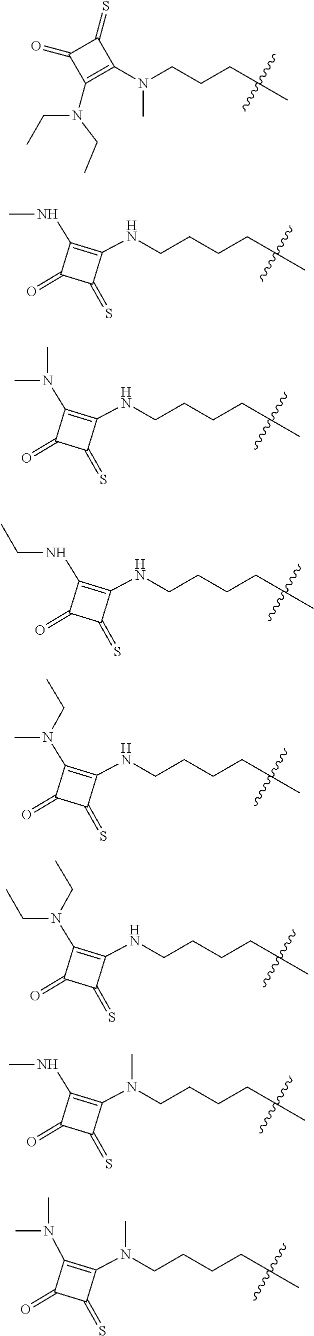

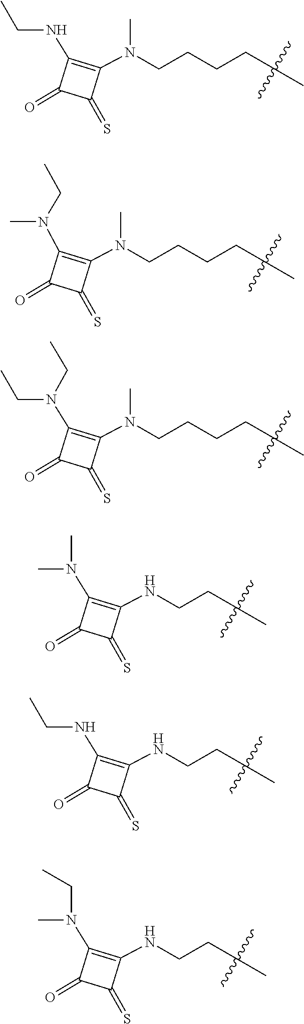 [SMILES: *CCCCN(C)c1c(NCC)c(=O)c1=S ; *CCCCN(C)c1c(N(C)CC)c(=O)c1=S ; *CCCCN(C)c1c(N(CC)CC)c(=O)c1=S ; *CCNc1c(N(C)C)c(=O)c1=S ; *CCNc1c(NCC)c(=O)c1=S ; *CCNc1c(N(C)CC)c(=O)c1=S]

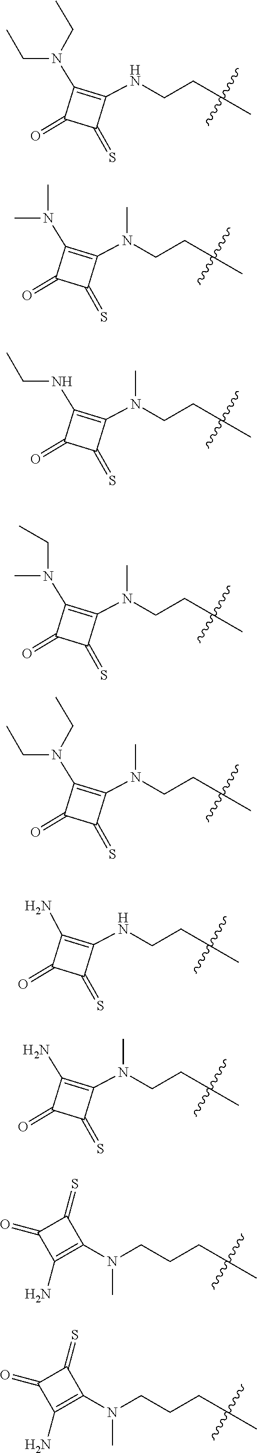

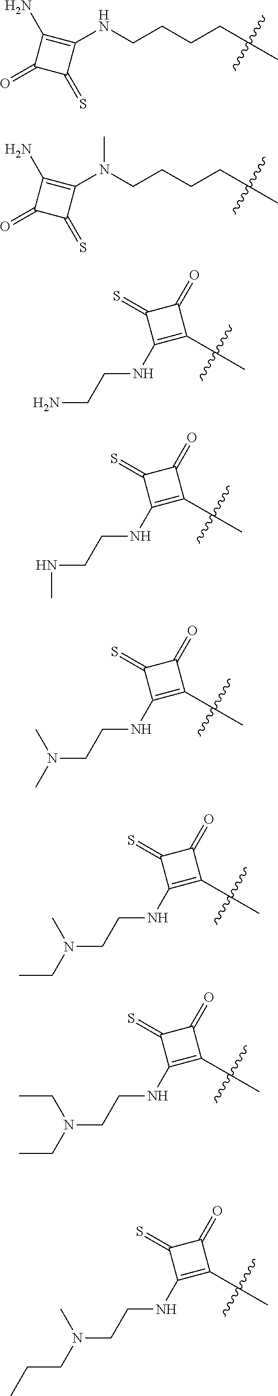

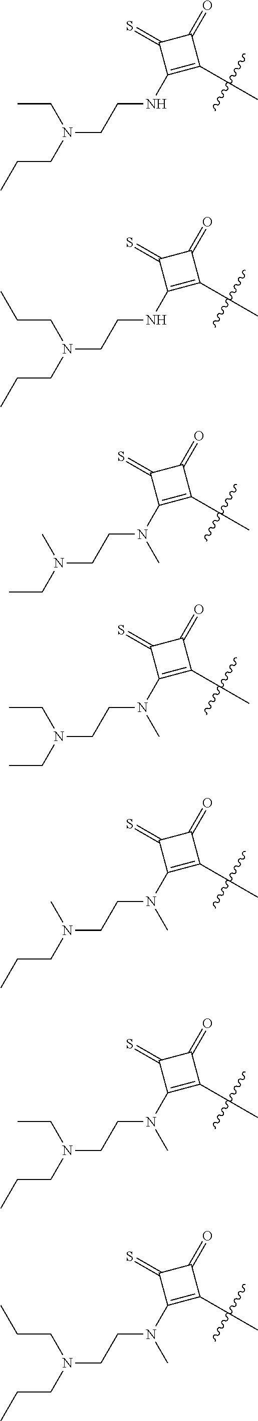

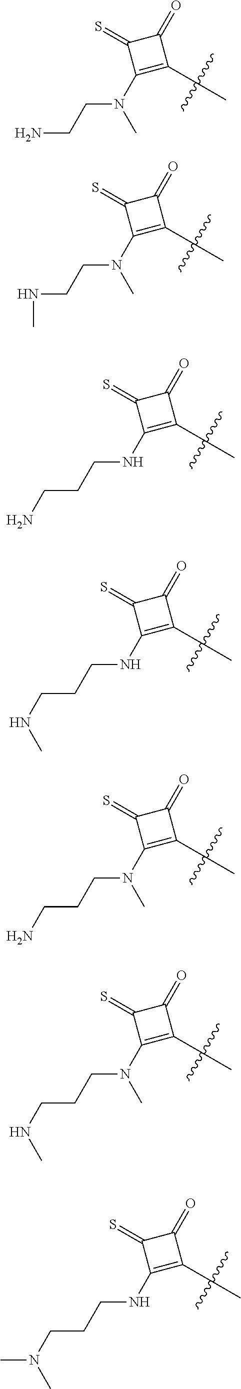

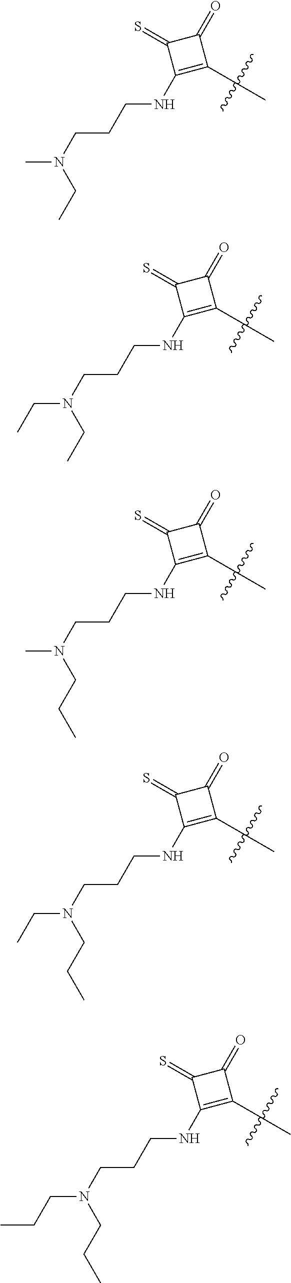 [SMILES: *c1c(NCCCN(C)CC)c(=S)c1=O ; *c1c(NCCCN(CC)CC)c(=S)c1=O ; *c1c(NCCCN(C)CCC)c(=S)c1=O ; *c1c(NCCCN(CC)CCC)c(=S)c1=O ; *c1c(NCCCN(CCC)CCC)c(=S)c1=O]

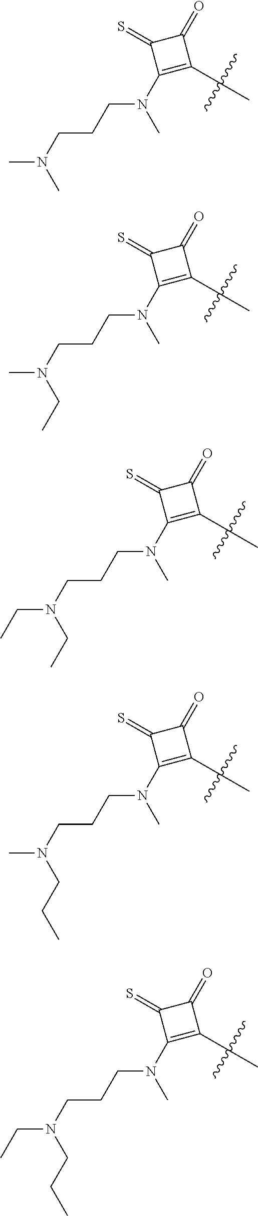 [SMILES: *c1c(N(C)CCCN(C)C)c(=S)c1=O ; *c1c(N(C)CCCN(C)CC)c(=S)c1=O ; *c1c(N(C)CCCN(CC)CC)c(=S)c1=O ; *c1c(N(C)CCCN(C)CCC)c(=S)c1=O ; *c1c(N(C)CCCN(CC)CCC)c(=S)c1=O]

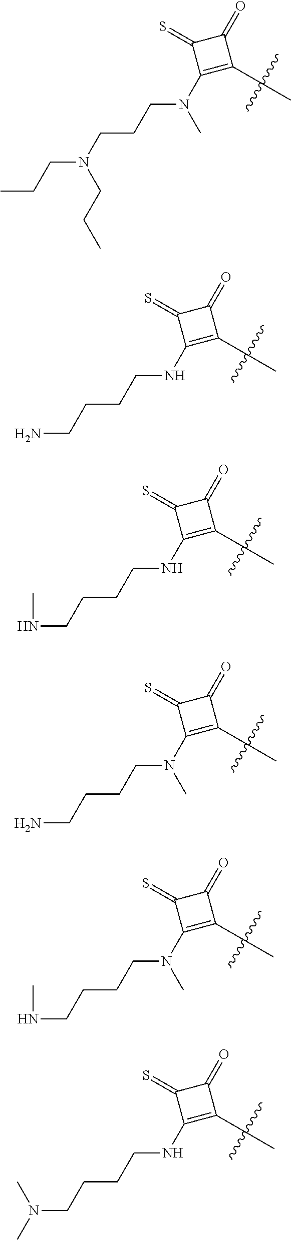 [SMILES: *c1c(N(C)CCCN(CCC)CCC)c(=S)c1=O ; *c1c(NCCCCN)c(=S)c1=O ; *c1c(NCCCCNC)c(=S)c1=O ; *c1c(N(C)CCCCN)c(=S)c1=O ; *c1c(N(C)CCCCNC)c(=S)c1=O ; *c1c(NCCCCN(C)C)c(=S)c1=O]

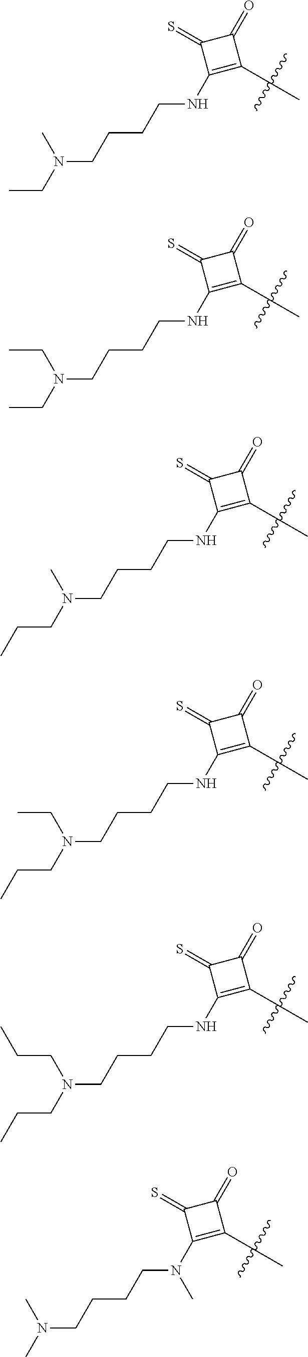 [SMILES: *c1c(NCCCCN(C)CC)c(=S)c1=O ; *c1c(NCCCCN(CC)CC)c(=S)c1=O ; *c1c(NCCCCN(C)CCC)c(=S)c1=O ; *c1c(NCCCCN(CC)CCC)c(=S)c1=O ; *c1c(NCCCCN(CCC)CCC)c(=S)c1=O ; *c1c(N(C)CCCCN(C)C)c(=S)c1=O]

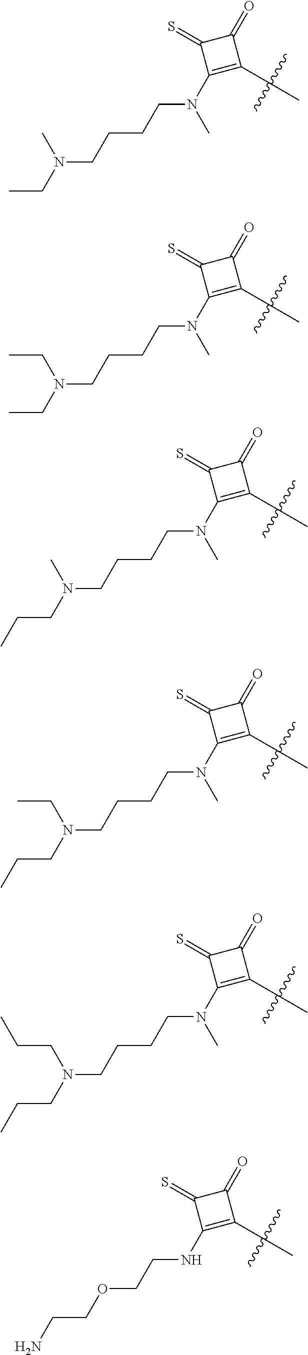 [SMILES: *c1c(N(C)CCCCN(C)CC)c(=S)c1=O ; *c1c(N(C)CCCCN(CC)CC)c(=S)c1=O ; *c1c(N(C)CCCCN(C)CCC)c(=S)c1=O ; *c1c(N(C)CCCCN(CC)CCC)c(=S)c1=O ; *c1c(N(C)CCCCN(CCC)CCC)c(=S)c1=O ; *c1c(NCCOCCN)c(=S)c1=O]

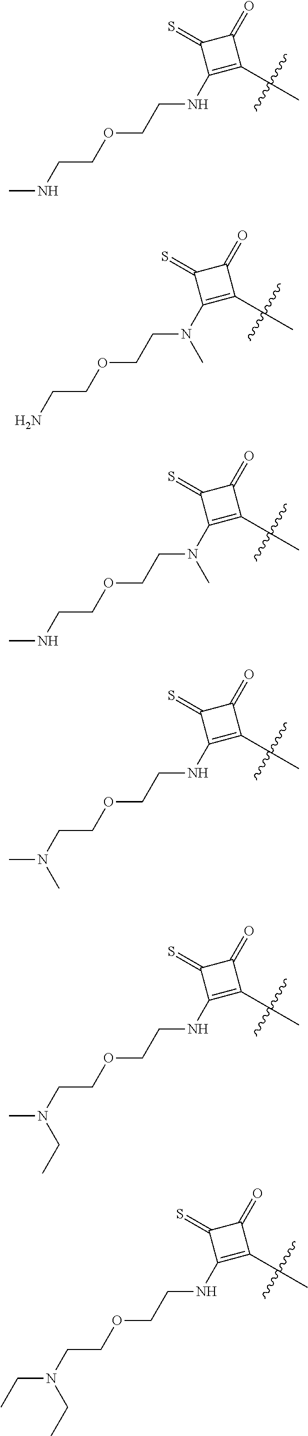 [SMILES: *c1c(NCCOCCNC)c(=S)c1=O ; *c1c(N(C)CCOCCN)c(=S)c1=O ; *c1c(N(C)CCOCCNC)c(=S)c1=O ; *c1c(NCCOCCN(C)C)c(=S)c1=O ; *c1c(NCCOCCN(C)CC)c(=S)c1=O ; *c1c(NCCOCCN(CC)CC)c(=S)c1=O]

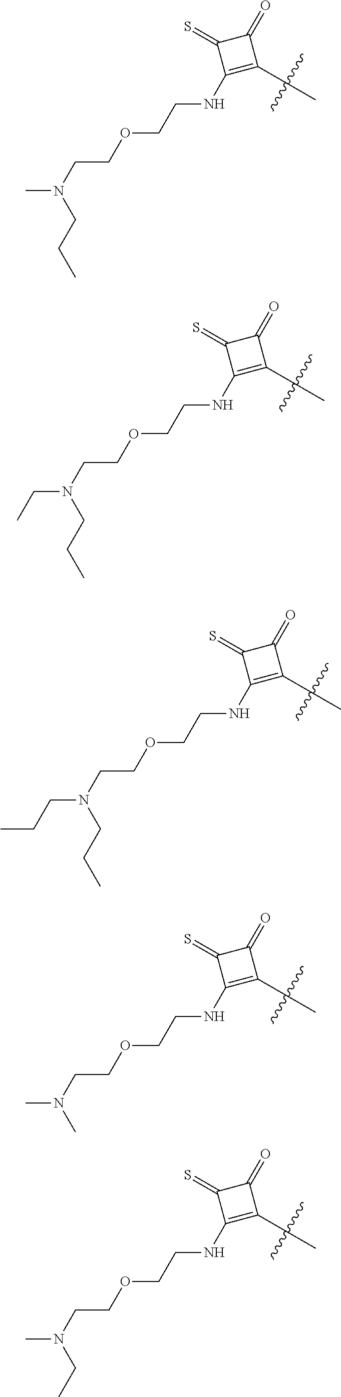 [SMILES: *c1c(NCCOCCN(C)CCC)c(=S)c1=O ; *c1c(NCCOCCN(CC)CCC)c(=S)c1=O ; *c1c(NCCOCCN(CCC)CCC)c(=S)c1=O ; *c1c(NCCOCCN(C)C)c(=S)c1=O ; *c1c(NCCOCCN(C)CC)c(=S)c1=O]

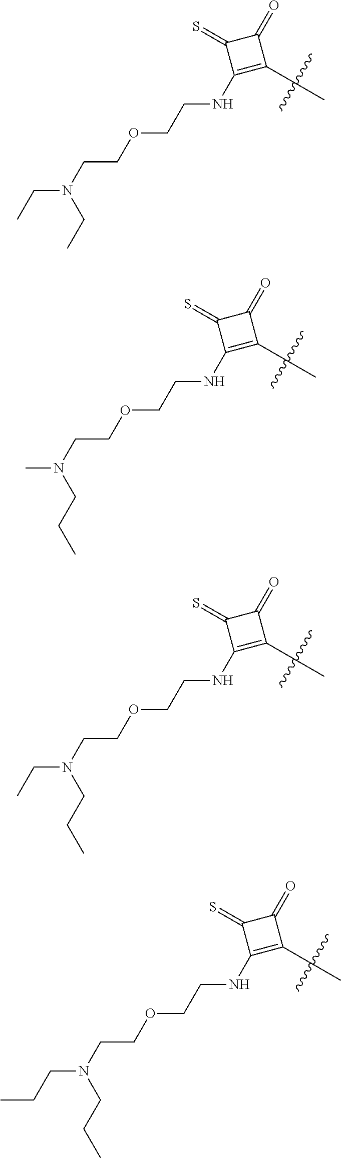 [SMILES: *c1c(NCCOCCN(CC)CC)c(=S)c1=O ; *c1c(NCCOCCN(C)CCC)c(=S)c1=O ; *c1c(NCCOCCN(CC)CCC)c(=S)c1=O ; *c1c(NCCOCCN(CCC)CCC)c(=S)c1=O]

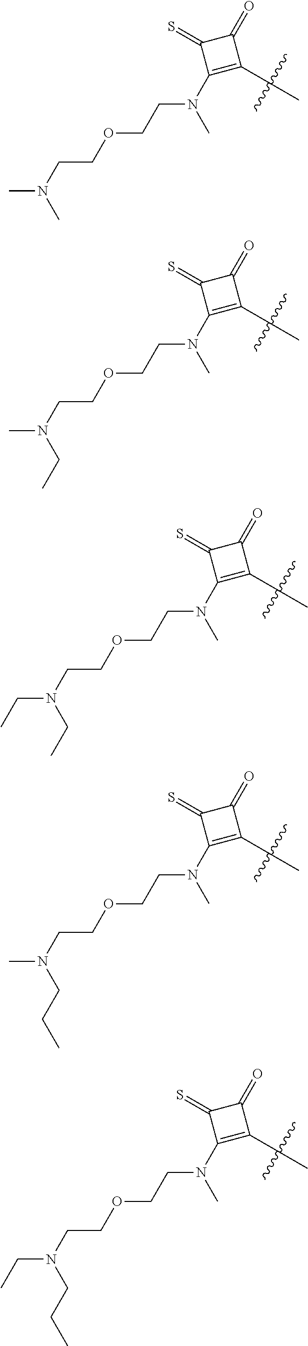 [SMILES: *c1c(N(C)CCOCCN(C)C)c(=S)c1=O ; *c1c(N(C)CCOCCN(C)CC)c(=S)c1=O ; *c1c(N(C)CCOCCN(CC)CC)c(=S)c1=O ; *c1c(N(C)CCOCCN(C)CCC)c(=S)c1=O ; *c1c(N(C)CCOCCN(CC)CCC)c(=S)c1=O]

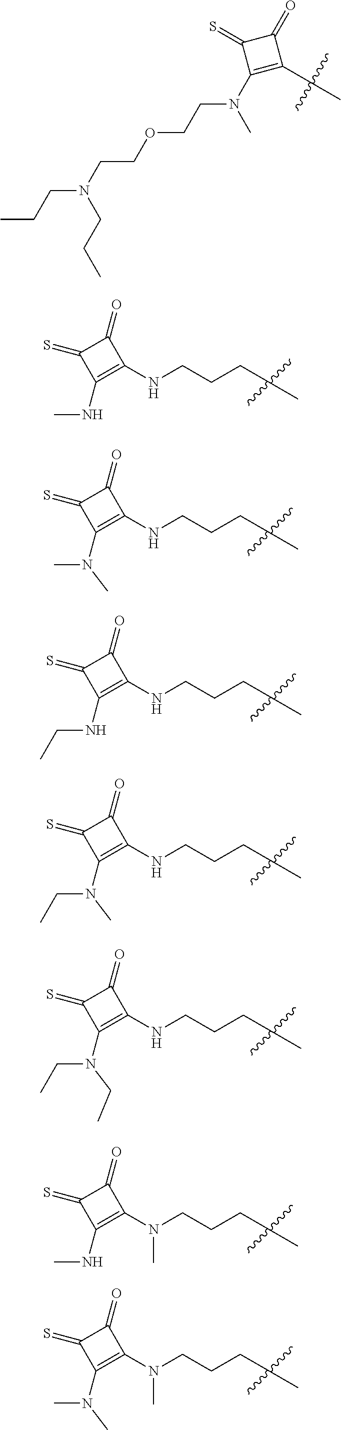

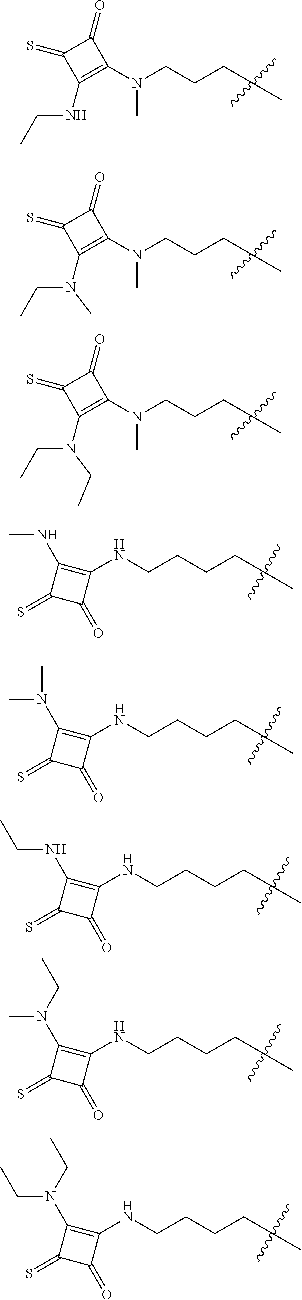

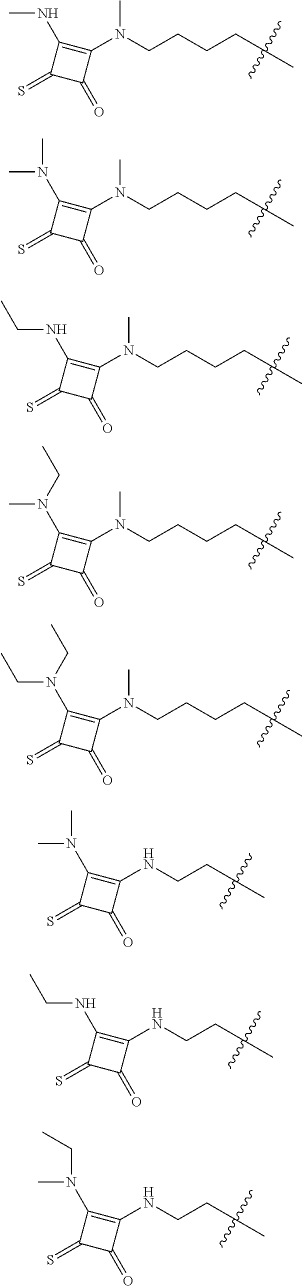

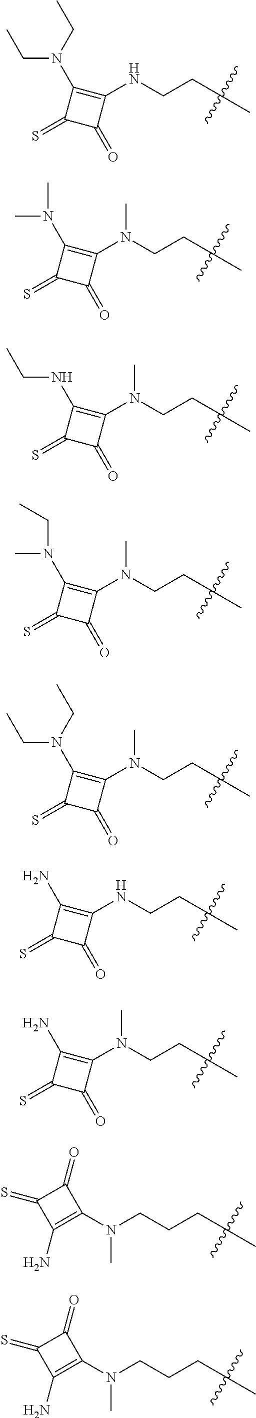

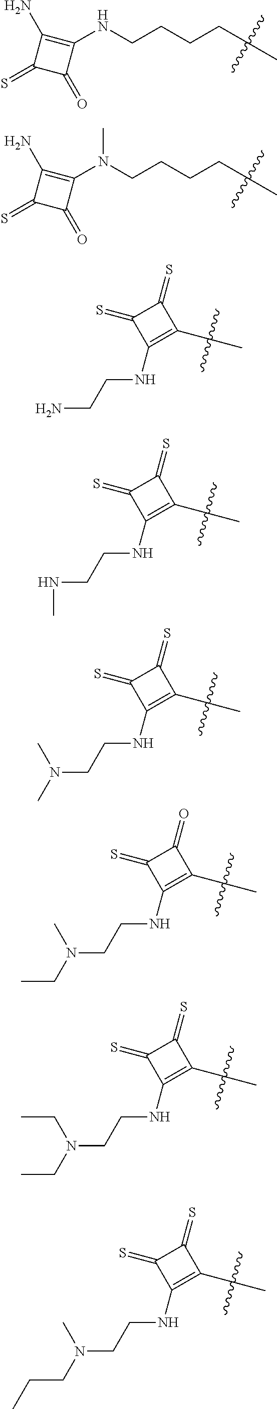

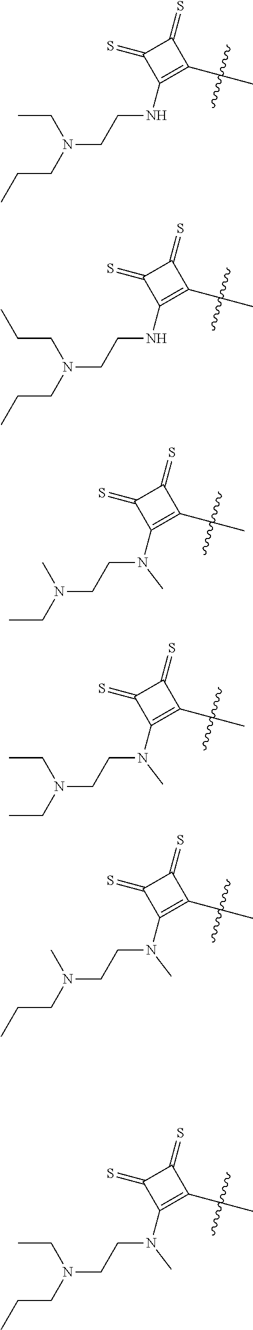 [SMILES: *c1c(NCCN(CC)CCC)c(=S)c1=S ; *c1c(NCCN(CCC)CCC)c(=S)c1=S ; *c1c(N(C)CCN(C)CC)c(=S)c1=S ; *c1c(N(C)CCN(CC)CC)c(=S)c1=S ; *c1c(N(C)CCN(C)CCC)c(=S)c1=S ; *c1c(N(C)CCN(CC)CCC)c(=S)c1=S]

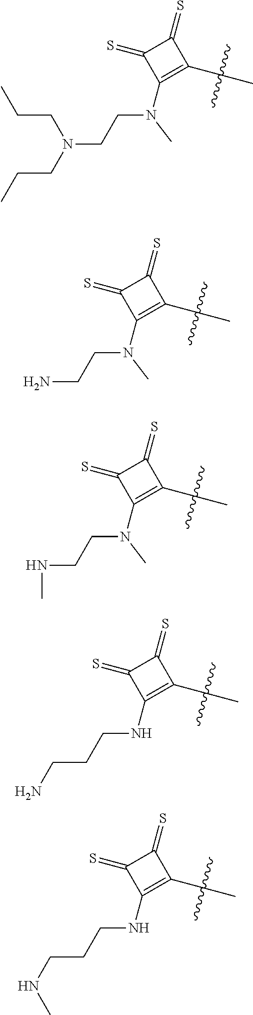 [SMILES: *c1c(N(C)CCN(CCC)CCC)c(=S)c1=S ; *c1c(N(C)CCN)c(=S)c1=S ; *c1c(N(C)CCNC)c(=S)c1=S ; *c1c(NCCCN)c(=S)c1=S ; *c1c(NCCCNC)c(=S)c1=S]

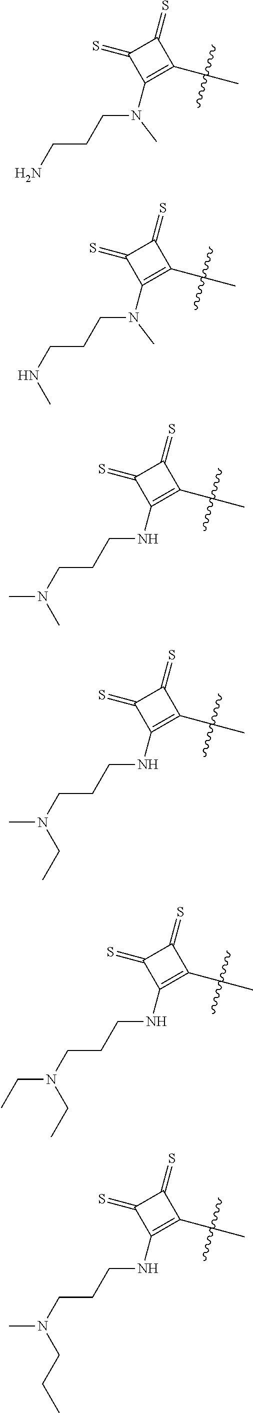 [SMILES: *c1c(N(C)CCCN)c(=S)c1=S ; *c1c(N(C)CCCNC)c(=S)c1=S ; *c1c(NCCCN(C)C)c(=S)c1=S ; *c1c(NCCCN(C)CC)c(=S)c1=S ; *c1c(NCCCN(CC)CC)c(=S)c1=S ; *c1c(NCCCN(C)CCC)c(=S)c1=S]

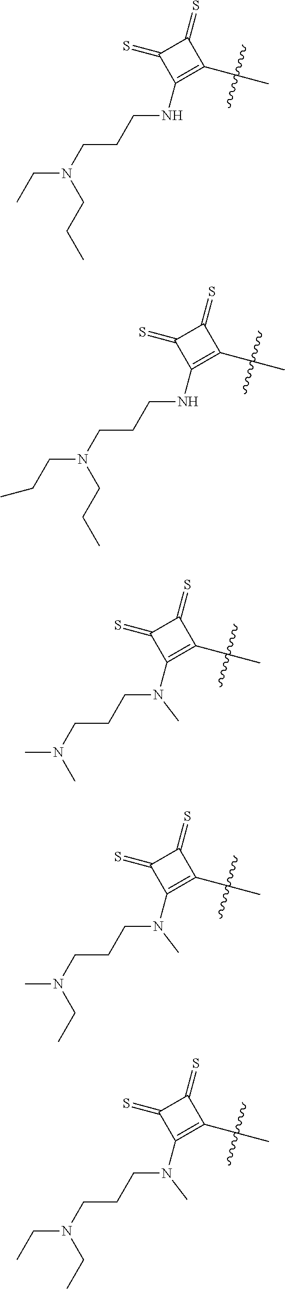 [SMILES: *c1c(NCCCN(CC)CCC)c(=S)c1=S ; *c1c(NCCCN(CCC)CCC)c(=S)c1=S ; *c1c(N(C)CCCN(C)C)c(=S)c1=S ; *c1c(N(C)CCCN(C)CC)c(=S)c1=S ; *c1c(N(C)CCCN(CC)CC)c(=S)c1=S]

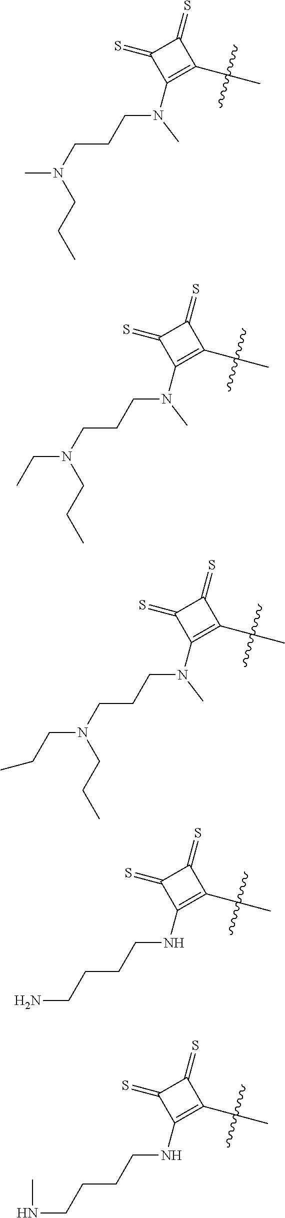 [SMILES: *c1c(N(C)CCCN(C)CCC)c(=S)c1=S ; *c1c(N(C)CCCN(CC)CCC)c(=S)c1=S ; *c1c(N(C)CCCN(CCC)CCC)c(=S)c1=S ; *c1c(NCCCCN)c(=S)c1=S ; *c1c(NCCCCNC)c(=S)c1=S]

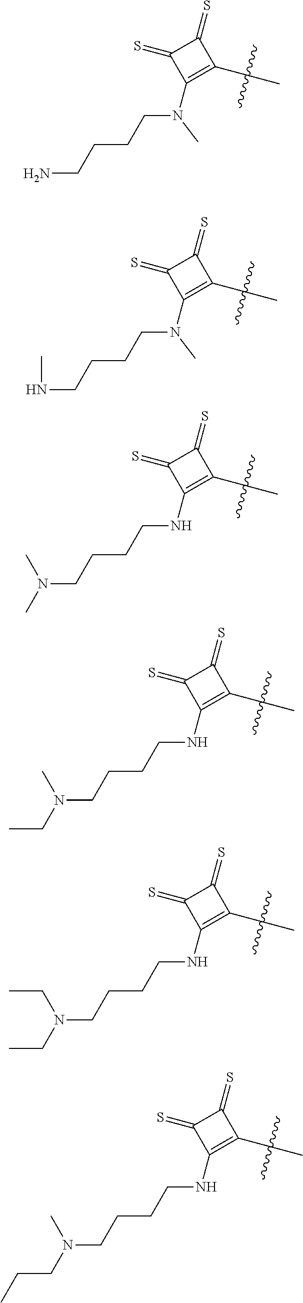 [SMILES: *c1c(N(C)CCCCN)c(=S)c1=S ; *c1c(N(C)CCCCNC)c(=S)c1=S ; *c1c(NCCCCN(C)C)c(=S)c1=S ; *c1c(NCCCCN(C)CC)c(=S)c1=S ; *c1c(NCCCCN(CC)CC)c(=S)c1=S ; *c1c(NCCCCN(C)CCC)c(=S)c1=S]

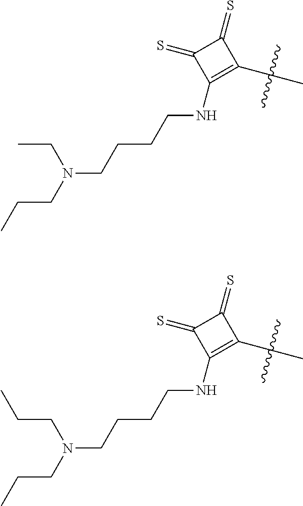 [SMILES: *c1c(NCCCCN(CC)CCC)c(=S)c1=S ; *c1c(NCCCCN(CCC)CCC)c(=S)c1=S]

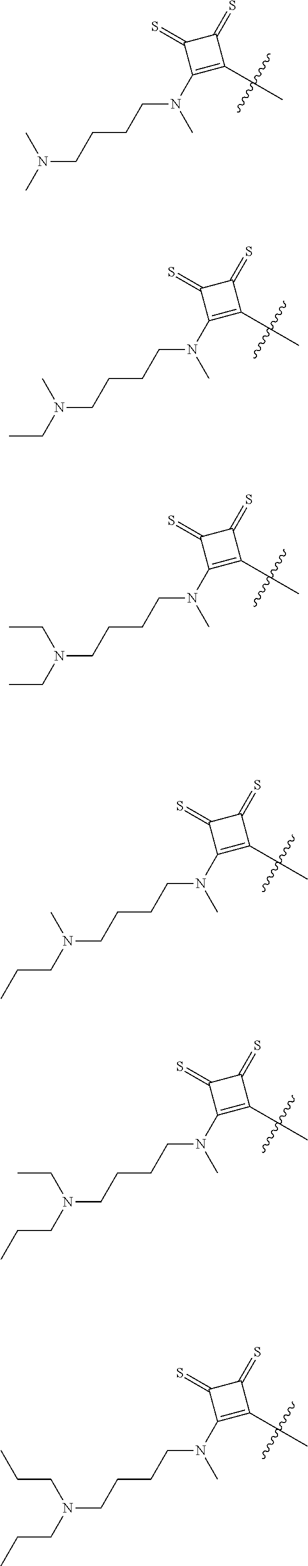 [SMILES: *c1c(N(C)CCCCN(C)C)c(=S)c1=S ; *c1c(N(C)CCCCN(C)CC)c(=S)c1=S ; *c1c(N(C)CCCCN(CC)CC)c(=S)c1=S ; *c1c(N(C)CCCCN(C)CCC)c(=S)c1=S ; *c1c(N(C)CCCCN(CC)CCC)c(=S)c1=S ; *c1c(N(C)CCCCN(CCC)CCC)c(=S)c1=S]

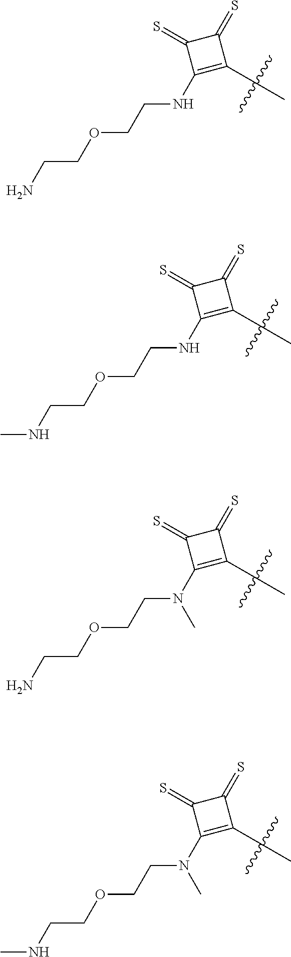 [SMILES: *c1c(NCCOCCN)c(=S)c1=S ; *c1c(NCCOCCNC)c(=S)c1=S ; *c1c(N(C)CCOCCN)c(=S)c1=S ; *c1c(N(C)CCOCCNC)c(=S)c1=S]

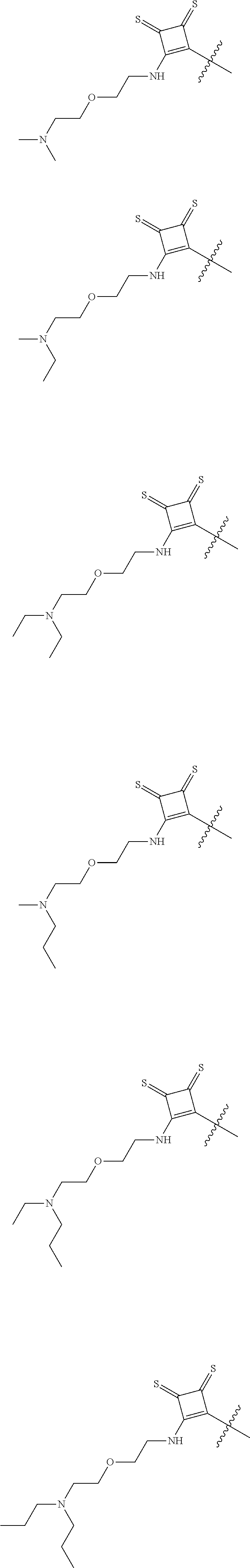 [SMILES: *c1c(NCCOCCN(C)C)c(=S)c1=S ; *c1c(NCCOCCN(C)CC)c(=S)c1=S ; *c1c(NCCOCCN(CC)CC)c(=S)c1=S ; *c1c(NCCOCCN(C)CCC)c(=S)c1=S ; *c1c(NCCOCCN(CC)CCC)c(=S)c1=S ; *c1c(NCCOCCN(CCC)CCC)c(=S)c1=S]

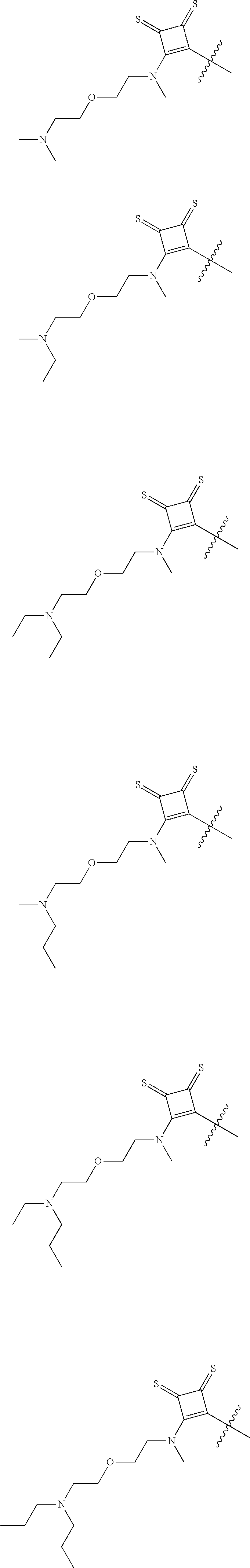 [SMILES: *c1c(N(C)CCOCCN(C)C)c(=S)c1=S ; *c1c(N(C)CCOCCN(C)CC)c(=S)c1=S ; *c1c(N(C)CCOCCN(CC)CC)c(=S)c1=S ; *c1c(N(C)CCOCCN(C)CCC)c(=S)c1=S ; *c1c(N(C)CCOCCN(CC)CCC)c(=S)c1=S ; *c1c(N(C)CCOCCN(CCC)CCC)c(=S)c1=S]

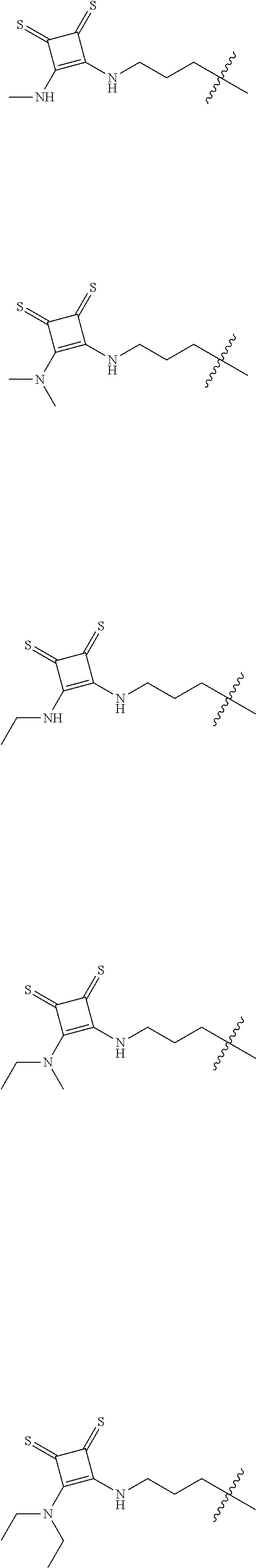 [SMILES: *CCCNc1c(NC)c(=S)c1=S ; *CCCNc1c(N(C)C)c(=S)c1=S ; *CCCNc1c(NCC)c(=S)c1=S ; *CCCNc1c(N(C)CC)c(=S)c1=S ; *CCCNc1c(N(CC)CC)c(=S)c1=S]

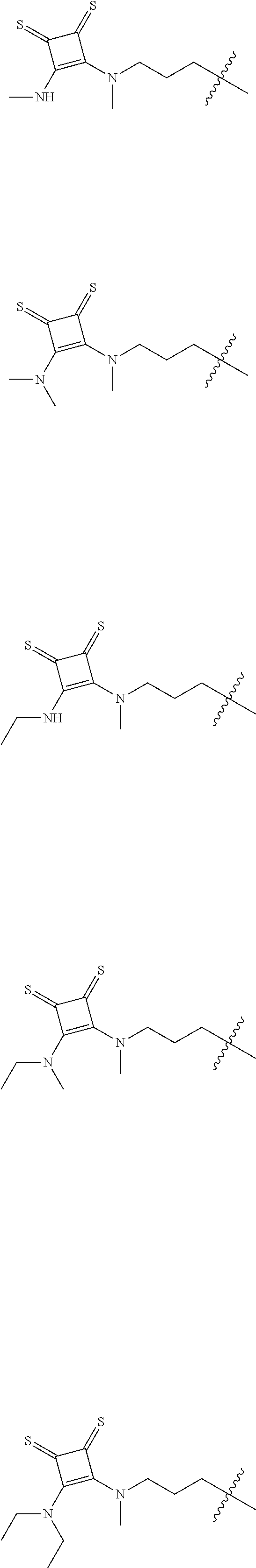 [SMILES: *CCCN(C)c1c(NC)c(=S)c1=S ; *CCCN(C)c1c(N(C)C)c(=S)c1=S ; *CCCN(C)c1c(NCC)c(=S)c1=S ; *CCCN(C)c1c(N(C)CC)c(=S)c1=S ; *CCCN(C)c1c(N(CC)CC)c(=S)c1=S]

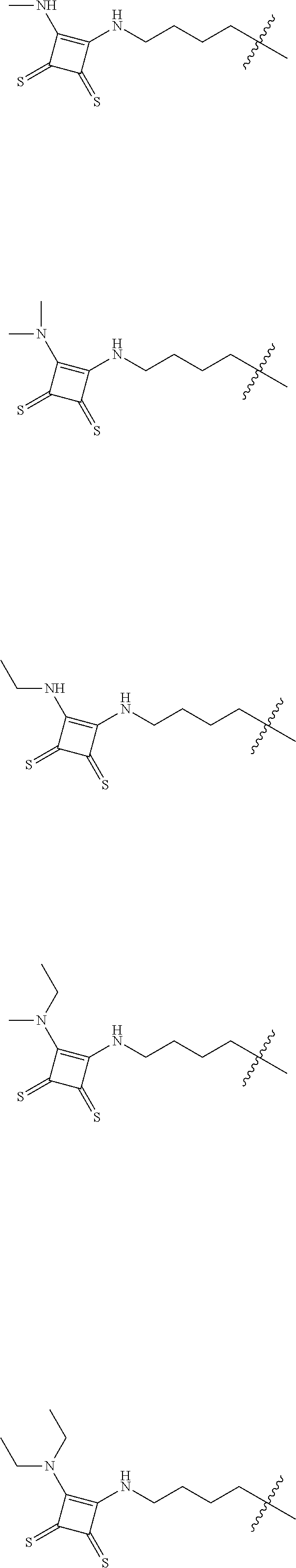 [SMILES: *CCCCNc1c(NC)c(=S)c1=S ; *CCCCNc1c(N(C)C)c(=S)c1=S ; *CCCCNc1c(NCC)c(=S)c1=S ; *CCCCNc1c(N(C)CC)c(=S)c1=S ; *CCCCNc1c(N(CC)CC)c(=S)c1=S]

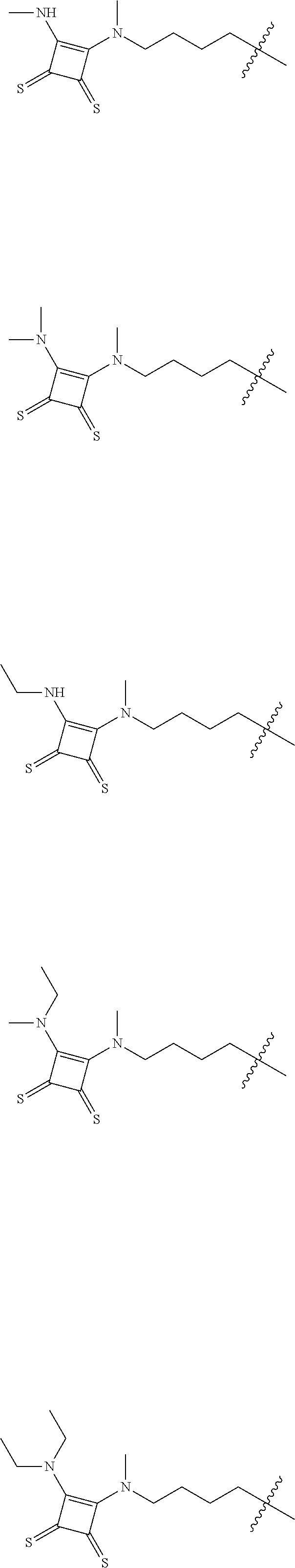 [SMILES: *CCCCN(C)c1c(NC)c(=S)c1=S ; *CCCCN(C)c1c(N(C)C)c(=S)c1=S ; *CCCCN(C)c1c(NCC)c(=S)c1=S ; *CCCCN(C)c1c(N(C)CC)c(=S)c1=S ; *CCCCN(C)c1c(N(CC)CC)c(=S)c1=S]

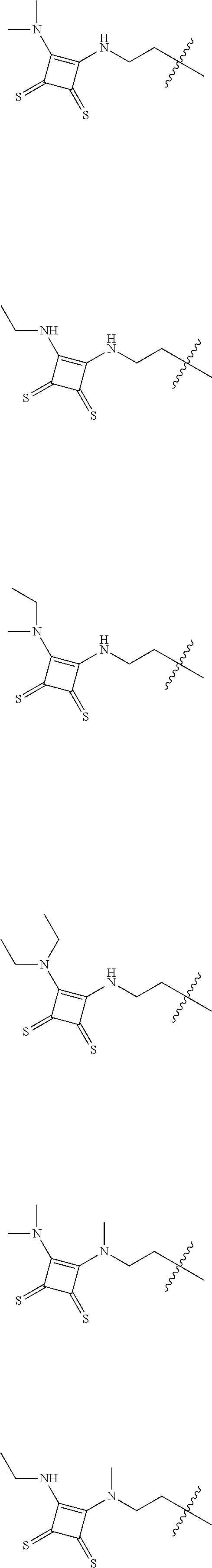 [SMILES: *CCNc1c(N(C)C)c(=S)c1=S ; *CCNc1c(NCC)c(=S)c1=S ; *CCNc1c(N(C)CC)c(=S)c1=S ; *CCNc1c(N(CC)CC)c(=S)c1=S ; *CCN(C)c1c(N(C)C)c(=S)c1=S ; *CCN(C)c1c(NCC)c(=S)c1=S]

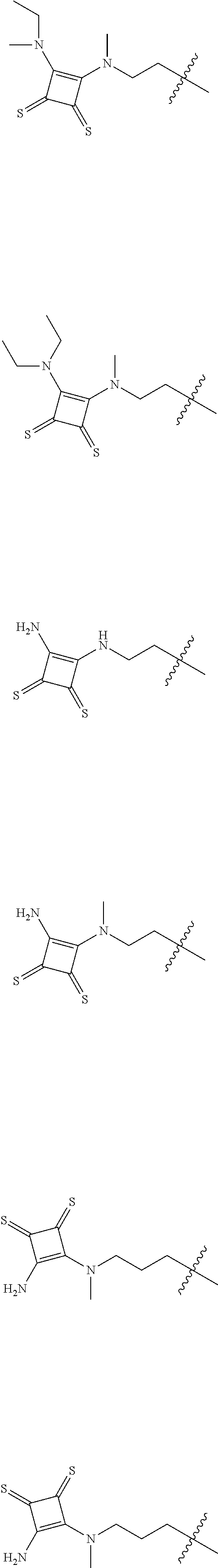 [SMILES: *CCN(C)c1c(N(C)CC)c(=S)c1=S ; *CCN(C)c1c(N(CC)CC)c(=S)c1=S ; *CCNc1c(N)c(=S)c1=S ; *CCN(C)c1c(N)c(=S)c1=S ; *CCCN(C)c1c(N)c(=S)c1=S ; *CCCN(C)c1c(N)c(=S)c1=S]

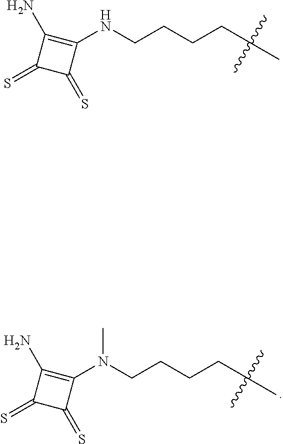 [SMILES: *CCCCNc1c(N)c(=S)c1=S ; *CCCCN(C)c1c(N)c(=S)c1=S]

In some embodiments, R⁴ is selected from any of the following groups:

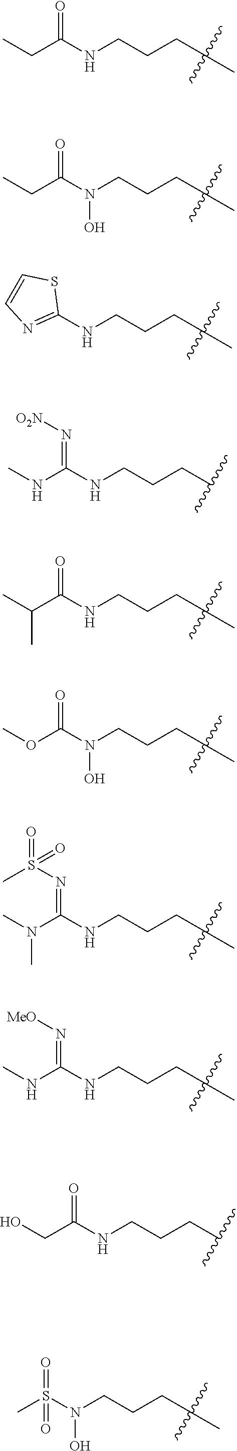

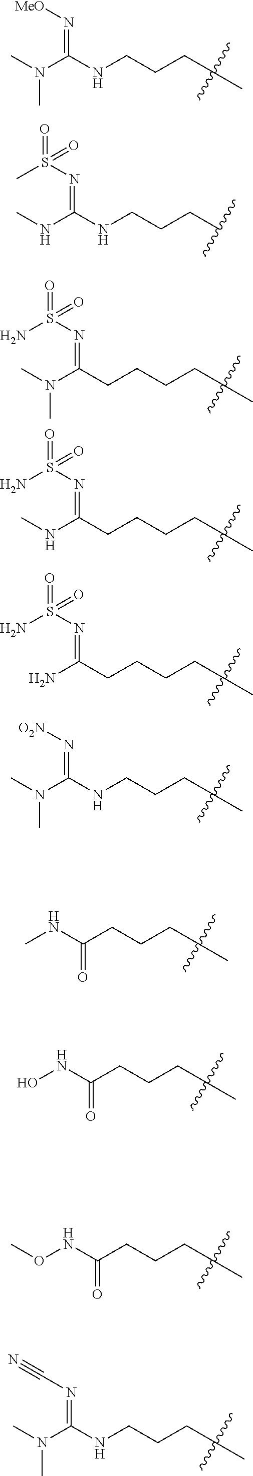

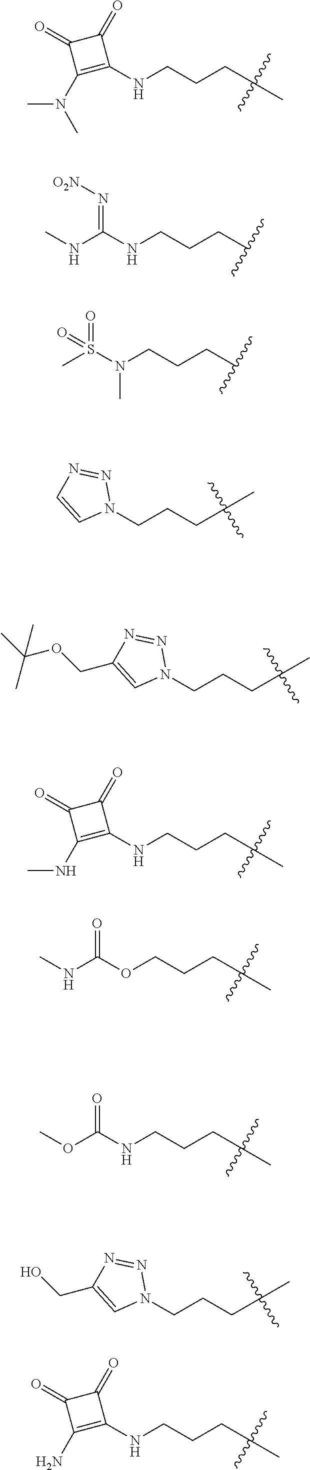

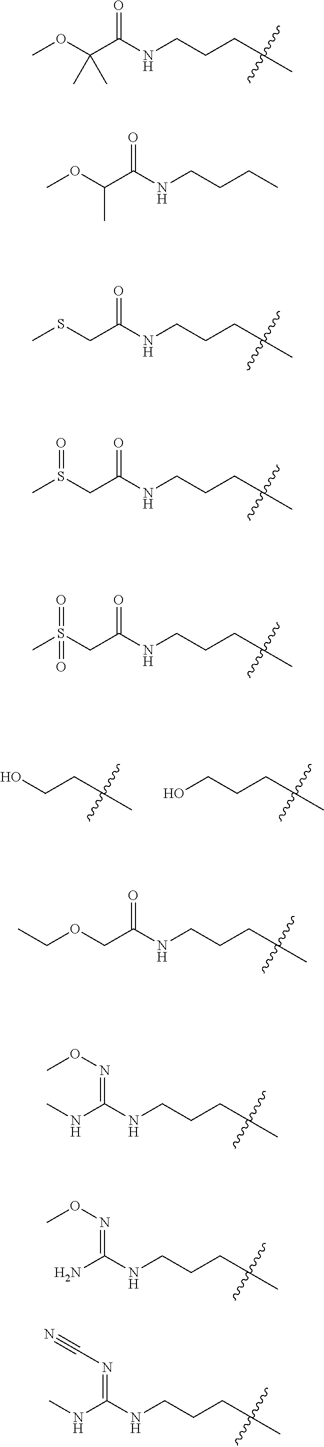

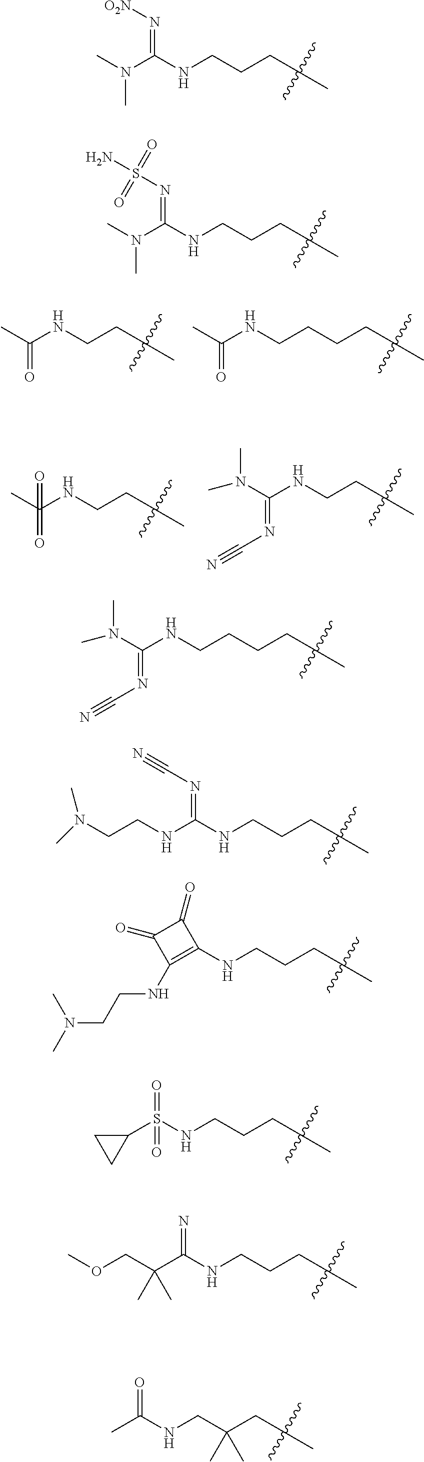

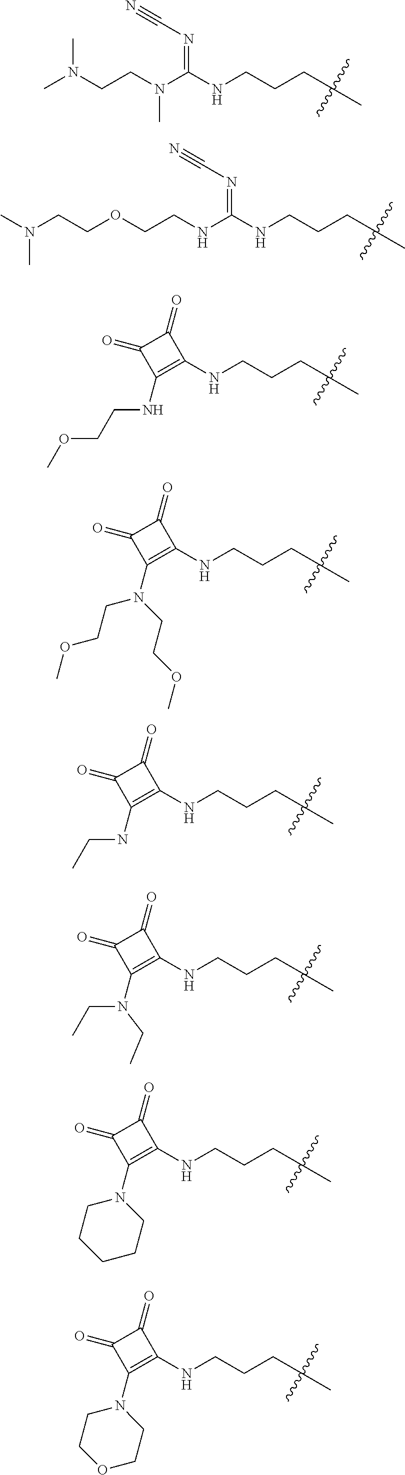

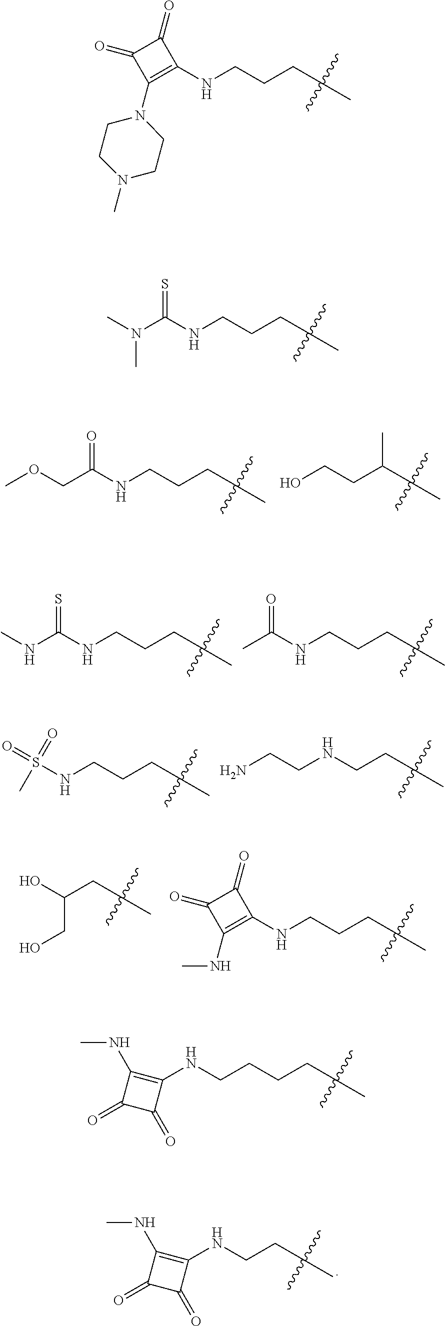

In some embodiments,

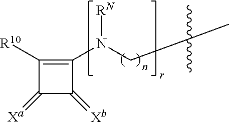

is selected from any of the following groups:

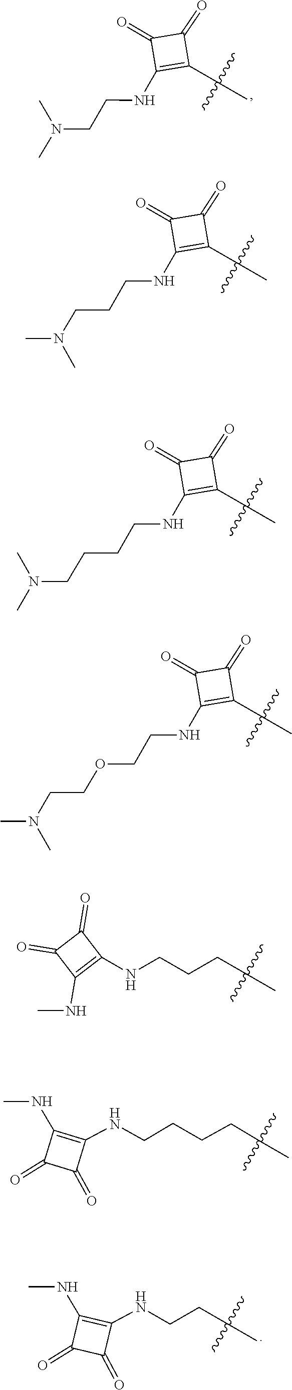

In some embodiments, a compound of Formula (III) further comprises an anion. As described herein, and anion can be any anion capable of reacting with an amine to form an ammonium salt. Examples include, but are not limited to, chloride, bromide, iodide, fluoride, acetate, formate, trifluoroacetate, difluoroacetate, trichloroacetate, and phosphate.

In some embodiments the compound of any of the formulae described herein is suitable for making a nanoparticle composition for intramuscular administration.

In some embodiments, R² and R³, together with the atom to which they are attached, form a heterocycle or carbocycle. In some embodiments, R² and R³, together with the atom to which they are attached, form a 5- to 14-membered aromatic or non-aromatic heterocycle having one or more heteroatoms selected from N, O, S, and P. In some embodiments, R² and R³, together with the atom to which they are attached, form an optionally substituted C₃₋₂₀ carbocycle (e.g., C₃₋₁₈ carbocycle, C₃₋₁₅ carbocycle, C₃₋₁₂ carbocycle, or C₃₋₁₀ carbocycle), either aromatic or non-aromatic. In some embodiments, R² and R³, together with the atom to which they are attached, form a C₃₋₆ carbocycle. In other embodiments, R² and R³, together with the atom to which they are attached, form a C₆ carbocycle, such as a cyclohexyl or phenyl group. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a cyclohexyl or phenyl group bearing one or more C₅ alkyl substitutions. In certain embodiments, the heterocycle or C₃₋₆ carbocycle formed by R² and R³, is substituted with a carbocycle groups. For example, R² and R³, together with the atom to which they are attached, may form a cyclohexyl or phenyl group that is substituted with cyclohexyl. In some embodiments, R² and R³, together with the atom to which they are attached, form a C₇₋₁₅ carbocycle, such as a cycloheptyl, cyclopentadecanyl, or naphthyl group.

In some embodiments, R⁴ is selected from —(CH₂)ₙQ and —(CH₂)ₙCHQR. In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(R)S(O)₂R⁸, —N(H)C(O)N(H)(R), —N(R)C(S)N(R)₂, —N(H)C(S)N(R)₂, —N(H)C(S)N(H)(R), and a heterocycle. In other embodiments, Q is selected from the group consisting of an imidazole, a pyrimidine, and a purine.

In some embodiments, R² and R³, together with the atom to which they are attached, form a heterocycle or carbocycle. In some embodiments, R² and R³, together with the atom to which they are attached, form a C₃₋₆ carbocycle. In some embodiments, R² and R³, together with the atom to which they are attached, form a C₆ carbocycle. In some embodiments, R² and R³, together with the atom to which they are attached, form a phenyl group. In some embodiments, R² and R³, together with the atom to which they are attached, form a cyclohexyl group. In some embodiments, R² and R³, together with the atom to which they are attached, form a heterocycle. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a phenyl group bearing one or more C₅ alkyl substitutions.

In some embodiments, at least one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at most one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at least one occurrence of R⁵ and R⁶ is methyl.

The compounds of any one of formula (VI), (VI-a), (VII), (VIIa), (VIIb), (VIIc), (VIId), (VIII), (VIIIa), (VIIIb), (VIIIc) or (VIIId) include one or more of the following features when applicable.

In some embodiments, r is 0. In some embodiments, r is 1.

In some embodiments, n is 2, 3, or 4. In some embodiments, n is 2. In some embodiments, n is 4. In some embodiments, n is not 3.

In some embodiments, R^{N} is H. In some embodiments, R^{N} is C₁₋₃ alkyl. For example, in some embodiments R^{N} is C₁ alkyl. For example, in some embodiments R^{N} is C₂ alkyl. For example, in some embodiments R^{N} is C₂ alkyl.

In some embodiments, X^{a} is O. In some embodiments, X^{a} is S. In some embodiments, X^{b} is O. In some embodiments, X^{b} is S.

In some embodiments, R¹⁰ is selected from the group consisting of N(R)₂, —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, and a heterocycle.

In some embodiments, R¹⁰ is selected from the group consisting of —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, and a heterocycle.

In some embodiments wherein R¹⁰ is —NH(CH₂)ON(R)₂, o is 2, 3, or 4.

In some embodiments wherein —NH(CH₂)ₚ₁O(CH₂)_{q1}N(R)₂, p¹ is 2. In some embodiments wherein —NH(CH₂)ₚ₁O(CH₂)_{q1}N(R)₂, q¹ is 2.

In some embodiments wherein R¹⁰ is —N((CH₂)ₛ₁OR)₂, s¹ is 2.

In some embodiments wherein R¹⁰ is —NH(CH₂)ON(R)₂, —NH(CH₂)ₚO(CH₂)_{q}N(R)₂, —NH(CH₂)ₛOR, or —N((CH₂)₅OR)₂, R is H or C₁-C₃ alkyl. For example, in some embodiments, R is C₁ alkyl. For example, in some embodiments, R is C₂ alkyl. For example, in some embodiments, R is H. For example, in some embodiments, R is H and one R is C₁-C₃ alkyl. For example, in some embodiments, R is H and one R is C₁ alkyl. For example, in some embodiments, R is H and one R is C₂ alkyl. In some embodiments wherein R¹⁰ is —NH(CH₂)ₜ₁N(R)₂, —NH(CH₂)ₚ₁O(CH₂)_{q1}N(R)₂, —NH(CH₂)ₛ₁OR, or —N((CH₂)ₛ₁OR)₂, each R is C₂-C₄ alkyl.

For example, in some embodiments, one R is H and one R is C₂-C₄ alkyl. In some embodiments, R¹⁰ is a heterocycle. For example, in some embodiments, R¹⁰ is morpholinyl. For example, in some embodiments, R¹⁰ is methyhlpiperazinyl.

In some embodiments, each occurrence of R⁵ and R⁶ is H.

In some embodiments, the compound of Formula (I) is selected from the group consisting of:

| Cpd | Structure |
|---|---|
| I 1 | 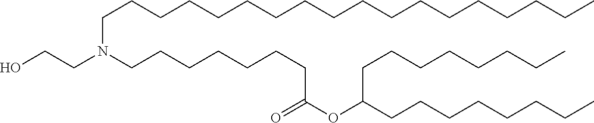 [SMILES: CCCCCCCCCCCCCCCCCCN(CCO)CCCCCCCC(=O)OC(CCCCCCCC)CCCCCCCC] |
| I 2 | 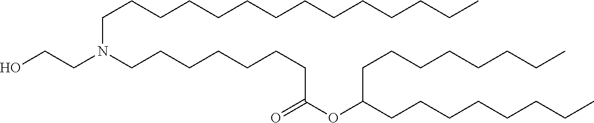 [SMILES: CCCCCCCCCCCCCCN(CCO)CCCCCCCC(=O)OC(CCCCCCCC)CCCCCCCC] |
| I 3 | 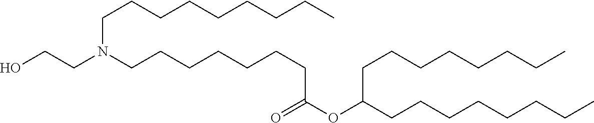 [SMILES: CCCCCCCCCN(CCO)CCCCCCCC(=O)OC(CCCCCCCC)CCCCCCCC] |
| I 4 | 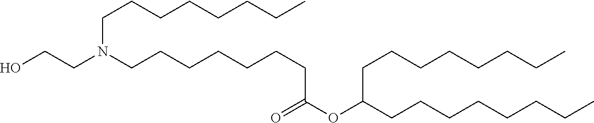 [SMILES: CCCCCCCCC(CCCCCCCC)OC(=O)CCCCCCCN(CCO)CCCCCCCC] |
| I 5 | 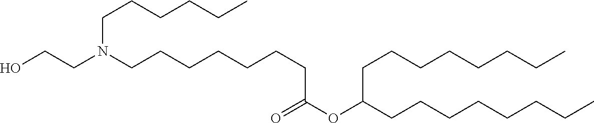 [SMILES: CCCCCCCCC(CCCCCCCC)OC(=O)CCCCCCCN(CCO)CCCCCC] |
| I 6 | 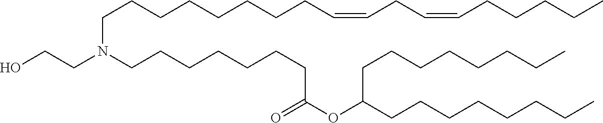 [SMILES: CCCCC/C=C\C/C=C\CCCCCCCCN(CCO)CCCCCCCC(=O)OC(CCCCCCCC)CCCCCCCC] |
| I 7 | 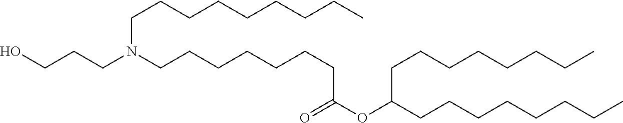 [SMILES: CCCCCCCCCN(CCCO)CCCCCCCC(=O)OC(CCCCCCCC)CCCCCCCC] |
| I 8 | 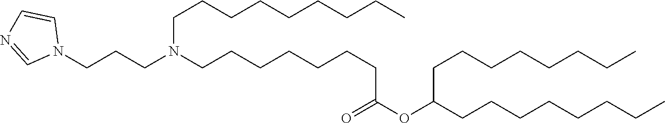 [SMILES: CCCCCCCCCN(CCCCCCCC(=O)OC(CCCCCCCC)CCCCCCCC)CCCn1ccnc1] |
| I 9 | 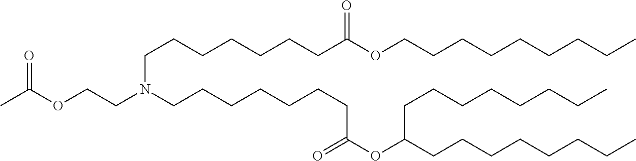 [SMILES: CCCCCCCCCOC(=O)CCCCCCCN(CCCCCCCC(=O)OC(CCCCCCCC)CCCCCCCC)CCOC(C)=O] |
| I 10 | 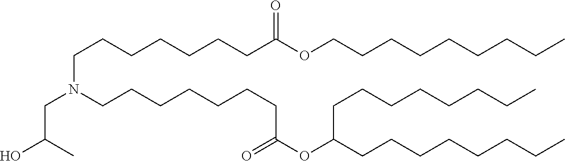 [SMILES: CCCCCCCCCOC(=O)CCCCCCCN(CCCCCCCC(=O)OC(CCCCCCCC)CCCCCCCC)CC(C)O] |
| I 11 | 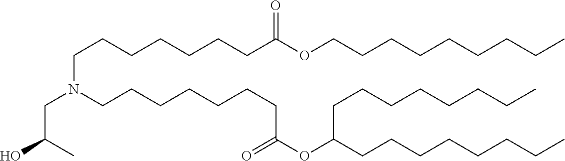 [SMILES: CCCCCCCCCOC(=O)CCCCCCCN(CCCCCCCC(=O)OC(CCCCCCCC)CCCCCCCC)C[C@@H](C)O] |
| I 12 | 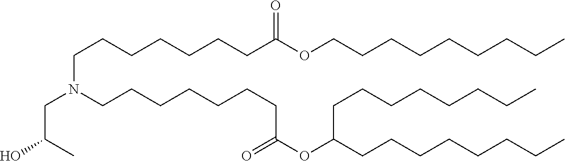 [SMILES: CCCCCCCCCOC(=O)CCCCCCCN(CCCCCCCC(=O)OC(CCCCCCCC)CCCCCCCC)C[C@H](C)O] |
| I 13 | 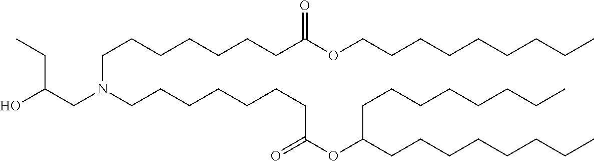 [SMILES: CCCCCCCCCOC(=O)CCCCCCCN(CCCCCCCC(=O)OC(CCCCCCCC)CCCCCCCC)CC(O)CC] |
| I 14 | 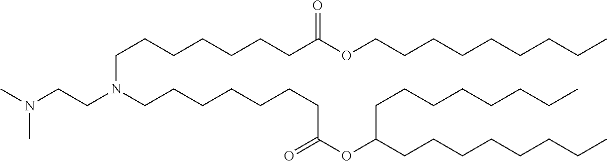 [SMILES: CCCCCCCCCOC(=O)CCCCCCCN(CCCCCCCC(=O)OC(CCCCCCCC)CCCCCCCC)CCN(C)C] |
| I 15 | 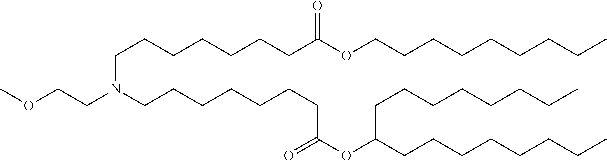 [SMILES: CCCCCCCCCOC(=O)CCCCCCCN(CCCCCCCC(=O)OC(CCCCCCCC)CCCCCCCC)CCOC] |
| I 16 | 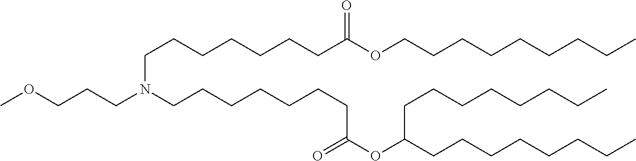 [SMILES: CCCCCCCCCOC(=O)CCCCCCCN(CCCCCCCC(=O)OC(CCCCCCCC)CCCCCCCC)CCCOC] |
| I 17 | 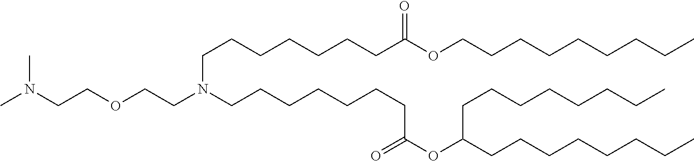 [SMILES: CCCCCCCCCOC(=O)CCCCCCCN(CCCCCCCC(=O)OC(CCCCCCCC)CCCCCCCC)CCOCCN(C)C] |
| I 18 | 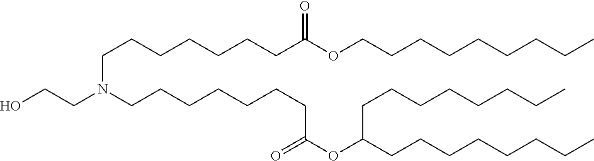 [SMILES: CCCCCCCCCOC(=O)CCCCCCCN(CCO)CCCCCCCC(=O)OC(CCCCCCCC)CCCCCCCC] |
| I 19 | 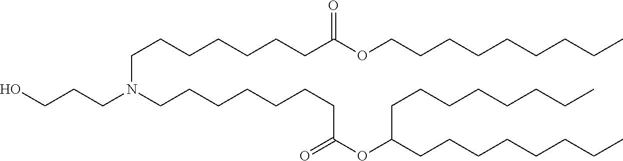 [SMILES: CCCCCCCCCOC(=O)CCCCCCCN(CCCO)CCCCCCCC(=O)OC(CCCCCCCC)CCCCCCCC] |
| I 20 | 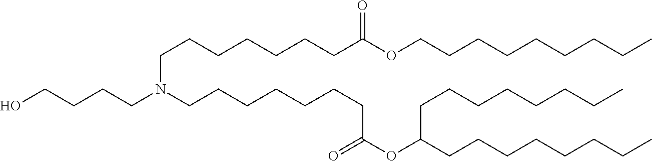 [SMILES: CCCCCCCCCOC(=O)CCCCCCCN(CCCCO)CCCCCCCC(=O)OC(CCCCCCCC)CCCCCCCC] |
| I 21 | 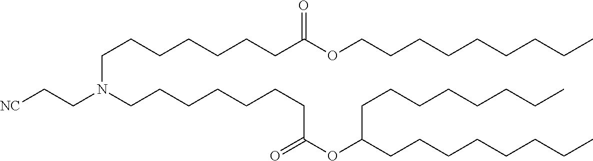 [SMILES: CCCCCCCCCOC(=O)CCCCCCCN(CCC#N)CCCCCCCC(=O)OC(CCCCCCCC)CCCCCCCC] |
| I 22 | 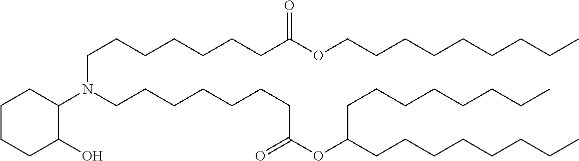 [SMILES: CCCCCCCCCOC(=O)CCCCCCCN(CCCCCCCC(=O)OC(CCCCCCCC)CCCCCCCC)C1CCCCC1O] |
| I 23 | 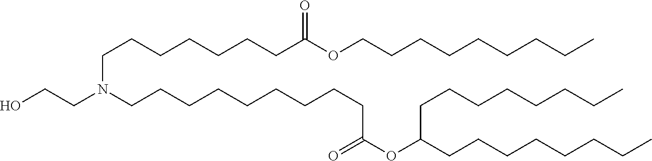 [SMILES: CCCCCCCCCOC(=O)CCCCCCCN(CCO)CCCCCCCCCC(=O)OC(CCCCCCCC)CCCCCCCC] |
| I 24 | 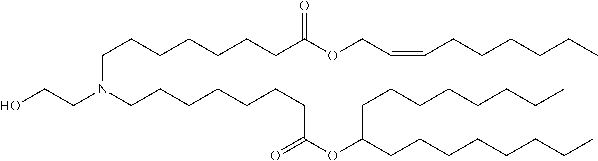 [SMILES: CCCCCC/C=C\COC(=O)CCCCCCCN(CCO)CCCCCCCC(=O)OC(CCCCCCCC)CCCCCCCC] |
| I 25 | 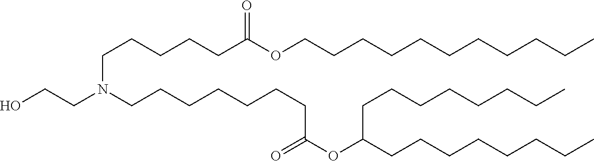 [SMILES: CCCCCCCCCCCOC(=O)CCCCCN(CCO)CCCCCCCC(=O)OC(CCCCCCCC)CCCCCCCC] |
| I 26 | 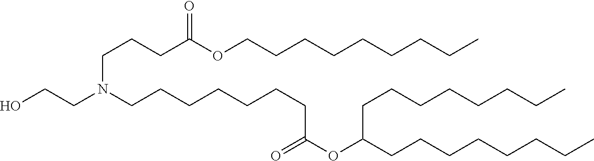 [SMILES: CCCCCCCCCOC(=O)CCCN(CCO)CCCCCCCC(=O)OC(CCCCCCCC)CCCCCCCC] |
| I 27 | 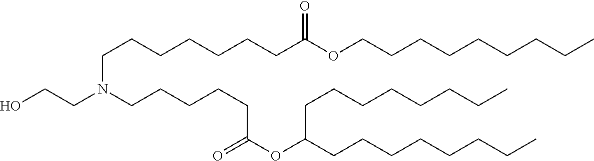 [SMILES: CCCCCCCCCOC(=O)CCCCCCCN(CCO)CCCCCC(=O)OC(CCCCCCCC)CCCCCCCC] |
| I 28 | 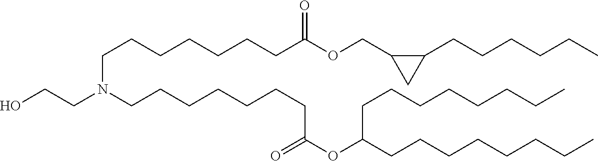 [SMILES: CCCCCCCCC(CCCCCCCC)OC(=O)CCCCCCCN(CCO)CCCCCCCC(=O)OCC1CC1CCCCCC] |
| I 29 | 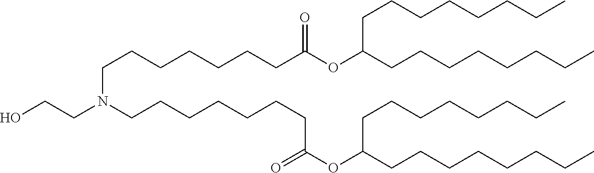 [SMILES: CCCCCCCCC(CCCCCCCC)OC(=O)CCCCCCCN(CCO)CCCCCCCC(=O)OC(CCCCCCCC)CCCCCCCC] |
| I 30 | 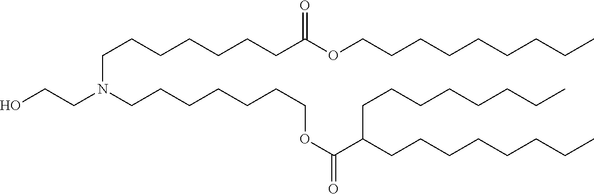 [SMILES: CCCCCCCCCOC(=O)CCCCCCCN(CCO)CCCCCCCOC(=O)C(CCCCCCCC)CCCCCCCC] |
| I 31 | 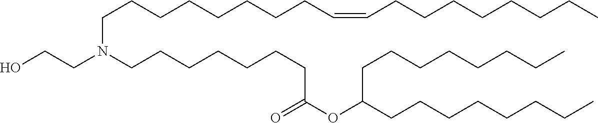 [SMILES: CCCCCCCC/C=C\CCCCCCCCN(CCO)CCCCCCCC(=O)OC(CCCCCCCC)CCCCCCCC] |
| I 32 | 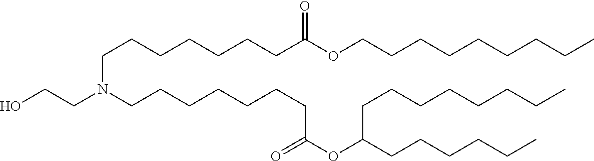 [SMILES: CCCCCCCCCOC(=O)CCCCCCCN(CCO)CCCCCCCC(=O)OC(CCCCCC)CCCCCCCC] |
| I 33 | 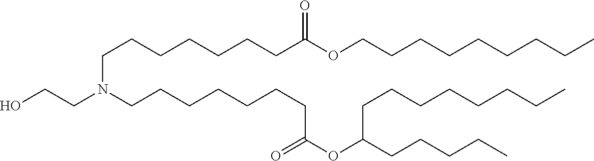 [SMILES: CCCCCCCCCOC(=O)CCCCCCCN(CCO)CCCCCCCC(=O)OC(CCCCC)CCCCCCCC] |
| I 34 | 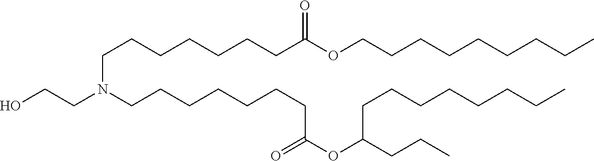 [SMILES: CCCCCCCCCOC(=O)CCCCCCCN(CCO)CCCCCCCC(=O)OC(CCC)CCCCCCCC] |
| I 35 | 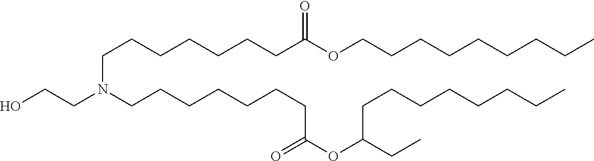 [SMILES: CCCCCCCCCOC(=O)CCCCCCCN(CCO)CCCCCCCC(=O)OC(CC)CCCCCCCC] |
| I 36 | 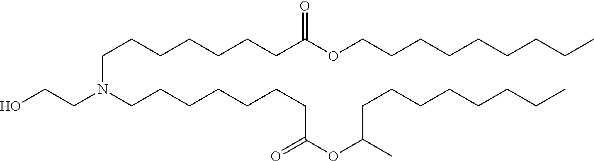 [SMILES: CCCCCCCCCOC(=O)CCCCCCCN(CCO)CCCCCCCC(=O)OC(C)CCCCCCCC] |
| I 37 | 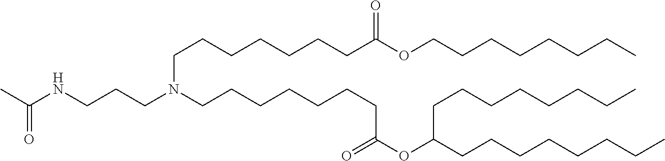 [SMILES: CCCCCCCCOC(=O)CCCCCCCN(CCCCCCCC(=O)OC(CCCCCCCC)CCCCCCCC)CCCNC(C)=O] |
| I 38 | 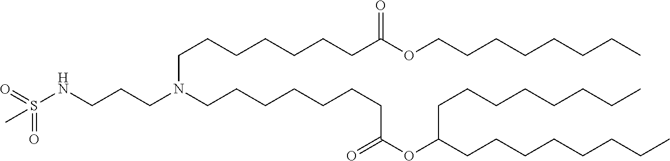 [SMILES: CCCCCCCCOC(=O)CCCCCCCN(CCCCCCCC(=O)OC(CCCCCCCC)CCCCCCCC)CCCNS(C)(=O)=O] |
| I 39 | 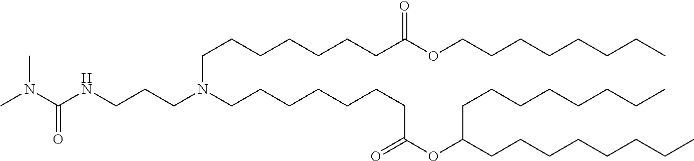 [SMILES: CCCCCCCCOC(=O)CCCCCCCN(CCCCCCCC(=O)OC(CCCCCCCC)CCCCCCCC)CCCNC(=O)N(C)C] |
| I 40 | 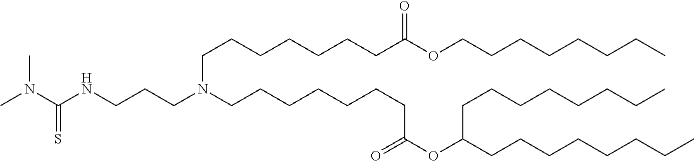 [SMILES: CCCCCCCCOC(=O)CCCCCCCN(CCCCCCCC(=O)OC(CCCCCCCC)CCCCCCCC)CCCNC(=S)N(C)C] |
| I 41 | 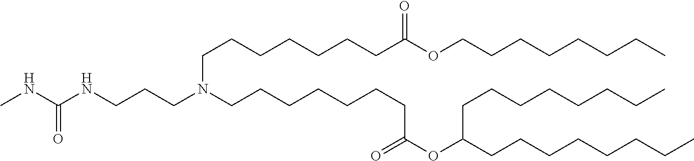 [SMILES: CCCCCCCCOC(=O)CCCCCCCN(CCCCCCCC(=O)OC(CCCCCCCC)CCCCCCCC)CCCNC(=O)NC] |
| I 42 | 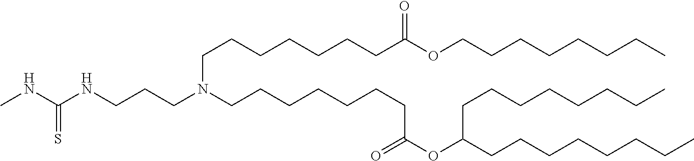 [SMILES: CCCCCCCCOC(=O)CCCCCCCN(CCCCCCCC(=O)OC(CCCCCCCC)CCCCCCCC)CCCNC(=S)NC] |
| I 43 | 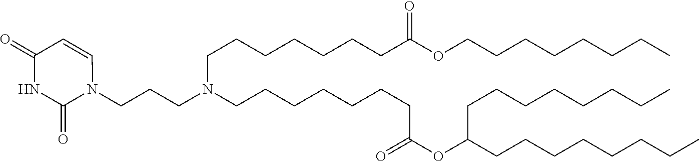 [SMILES: CCCCCCCCOC(=O)CCCCCCCN(CCCCCCCC(=O)OC(CCCCCCCC)CCCCCCCC)CCCn1ccc(=O)[nH]c1=O] |
| I 44 | 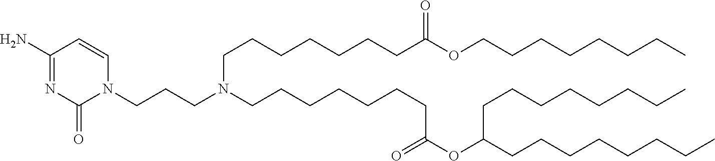 [SMILES: CCCCCCCCOC(=O)CCCCCCCN(CCCCCCCC(=O)OC(CCCCCCCC)CCCCCCCC)CCCn1ccc(N)nc1=O] |
| I 45 | 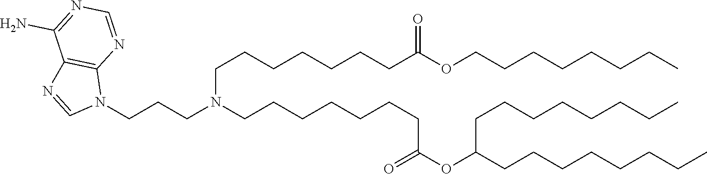 [SMILES: CCCCCCCCOC(=O)CCCCCCCN(CCCCCCCC(=O)OC(CCCCCCCC)CCCCCCCC)CCCn1cnc2c(N)ncnc21] |
| I 46 | 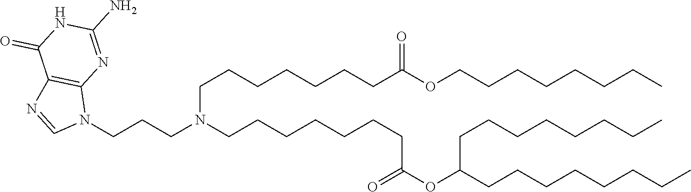 [SMILES: CCCCCCCCOC(=O)CCCCCCCN(CCCCCCCC(=O)OC(CCCCCCCC)CCCCCCCC)CCCn1cnc2c(=O)[nH]c(N)nc21] |
| I 47 | 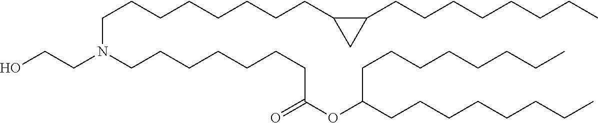 [SMILES: CCCCCCCCC(CCCCCCCC)OC(=O)CCCCCCCN(CCO)CCCCCCCCC1CC1CCCCCCCC] |
| I 48 | 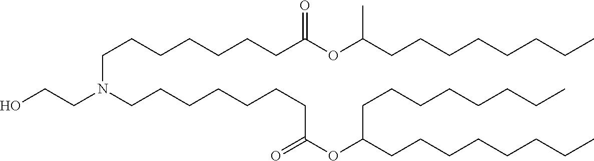 [SMILES: CCCCCCCCC(C)OC(=O)CCCCCCCN(CCO)CCCCCCCC(=O)OC(CCCCCCCC)CCCCCCCC] |
| I 49 | 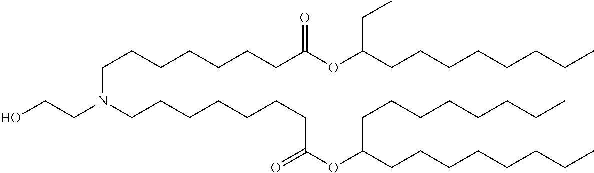 [SMILES: CCCCCCCCC(CC)OC(=O)CCCCCCCN(CCO)CCCCCCCC(=O)OC(CCCCCCCC)CCCCCCCC] |
| I 50 | 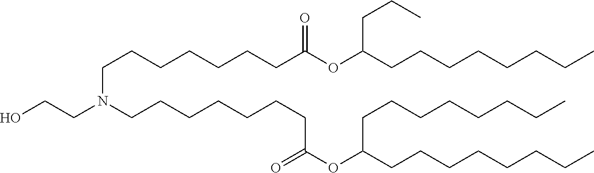 [SMILES: CCCCCCCCC(CCC)OC(=O)CCCCCCCN(CCO)CCCCCCCC(=O)OC(CCCCCCCC)CCCCCCCC] |
| I 51 | 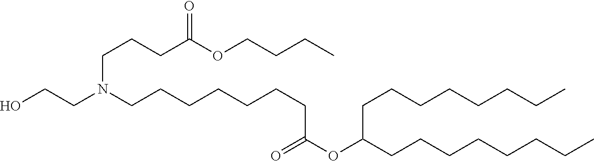 [SMILES: CCCCCCCCC(CCCCCCCC)OC(=O)CCCCCCCN(CCO)CCCC(=O)OCCCC] |
| I 52 | 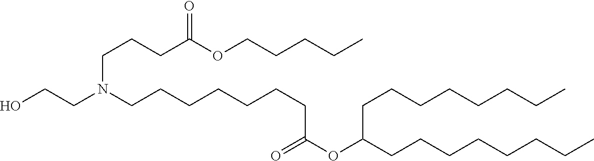 [SMILES: CCCCCCCCC(CCCCCCCC)OC(=O)CCCCCCCN(CCO)CCCC(=O)OCCCCC] |
| I 53 | 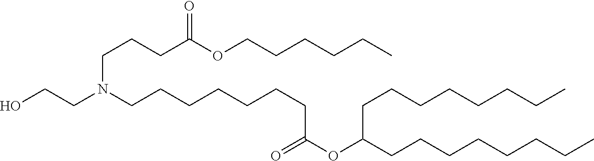 [SMILES: CCCCCCCCC(CCCCCCCC)OC(=O)CCCCCCCN(CCO)CCCC(=O)OCCCCCC] |
| I 54 | 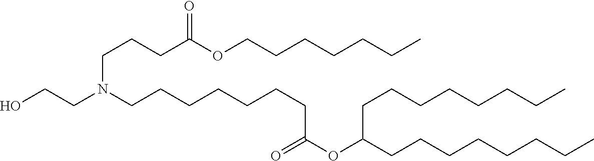 [SMILES: CCCCCCCCC(CCCCCCCC)OC(=O)CCCCCCCN(CCO)CCCC(=O)OCCCCCCC] |
| I 55 | 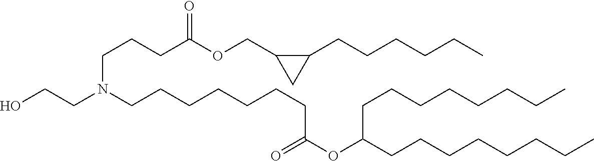 [SMILES: CCCCCCCCC(CCCCCCCC)OC(=O)CCCCCCCN(CCO)CCCC(=O)OCC1CC1CCCCCC] |
| I 56 | 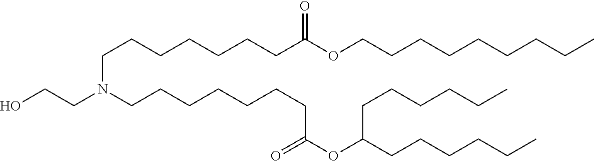 [SMILES: CCCCCCCCCOC(=O)CCCCCCCN(CCO)CCCCCCCC(=O)OC(CCCCCC)CCCCCC] |
| I 57 | 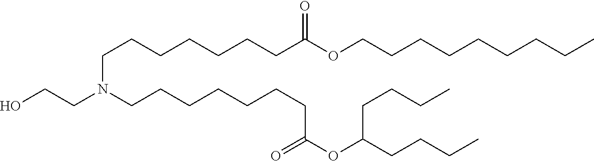 [SMILES: CCCCCCCCCOC(=O)CCCCCCCN(CCO)CCCCCCCC(=O)OC(CCCC)CCCC] |
| I 58 | 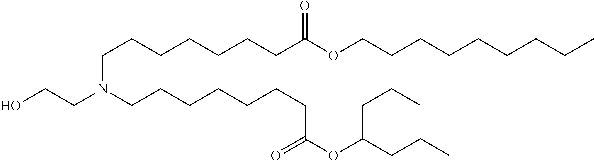 [SMILES: CCCCCCCCCOC(=O)CCCCCCCN(CCO)CCCCCCCC(=O)OC(CCC)CCC] |
| I 59 | 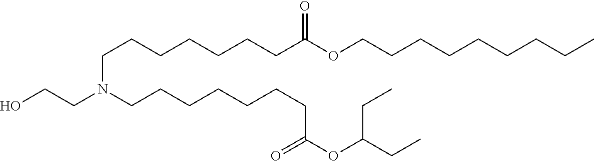 [SMILES: CCCCCCCCCOC(=O)CCCCCCCN(CCO)CCCCCCCC(=O)OC(CC)CC] |
| I 60 | 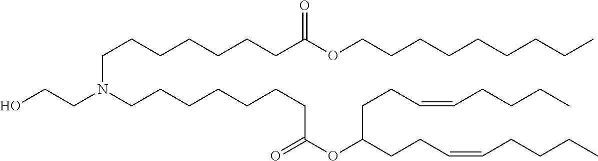 [SMILES: CCCC/C=C\CCC(CC/C=C\CCCC)OC(=O)CCCCCCCN(CCO)CCCCCCCC(=O)OCCCCCCCCC] |
| I 61 | 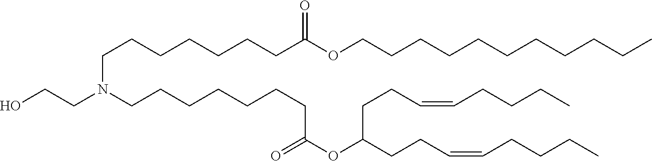 [SMILES: CCCC/C=C\CCC(CC/C=C\CCCC)OC(=O)CCCCCCCN(CCO)CCCCCCCC(=O)OCCCCCCCCCCC] |

In further embodiments, the compound of Formula (I I) is selected from the group consisting of:

| Cpd | Structure |
|---|---|
| I 62 | 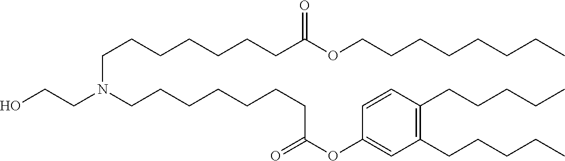 [SMILES: CCCCCCCCOC(=O)CCCCCCCN(CCO)CCCCCCCC(=O)Oc1ccc(CCCCC)c(CCCCC)c1] |
| I 63 | 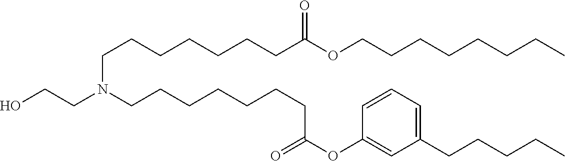 [SMILES: CCCCCCCCOC(=O)CCCCCCCN(CCO)CCCCCCCC(=O)Oc1cccc(CCCCC)c1] |
| I 64 | 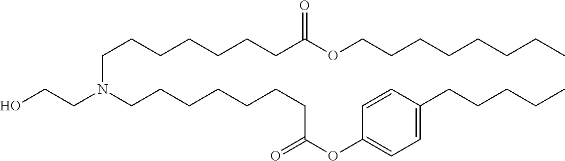 [SMILES: CCCCCCCCOC(=O)CCCCCCCN(CCO)CCCCCCCC(=O)Oc1ccc(CCCCC)cc1] |

In some embodiments, the compound of Formula (I I) or Formula (I IV) is selected from the group consisting of:

| Cpd | Structure |
|---|---|
| I 65 | 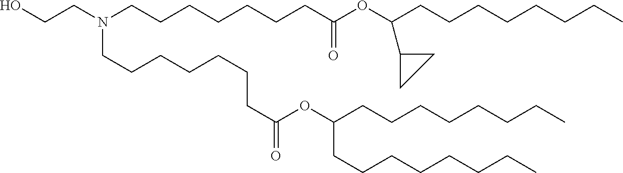 [SMILES: CCCCCCCCC(CCCCCCCC)OC(=O)CCCCCCCN(CCO)CCCCCCCC(=O)OC(CCCCCCCC)C1CC1] |
| I 66 | 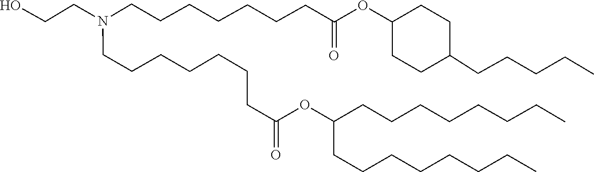 [SMILES: CCCCCCCCC(CCCCCCCC)OC(=O)CCCCCCCN(CCO)CCCCCCCC(=O)OC1CCC(CCCCC)CC1] |
| I 67 | 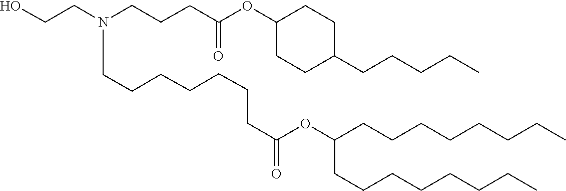 [SMILES: CCCCCCCCC(CCCCCCCC)OC(=O)CCCCCCCN(CCO)CCCC(=O)OC1CCC(CCCCC)CC1] |
| I 68 | 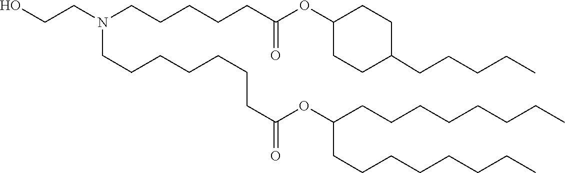 [SMILES: CCCCCCCCC(CCCCCCCC)OC(=O)CCCCCCCN(CCO)CCCCCC(=O)OC1CCC(CCCCC)CC1] |
| I 69 | 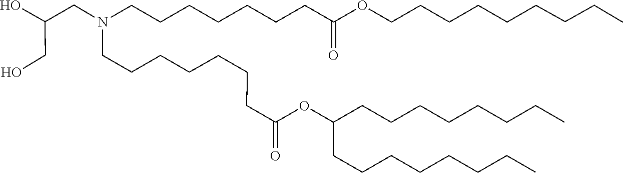 [SMILES: CCCCCCCCCOC(=O)CCCCCCCN(CCCCCCCC(=O)OC(CCCCCCCC)CCCCCCCC)CC(O)CO] |
| I 70 | 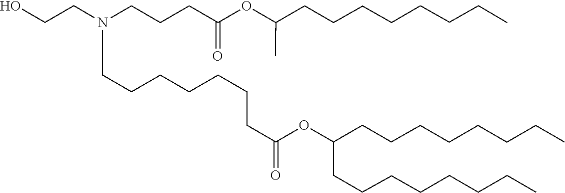 [SMILES: CCCCCCCCC(C)OC(=O)CCCN(CCO)CCCCCCCC(=O)OC(CCCCCCCC)CCCCCCCC] |
| I 71 | 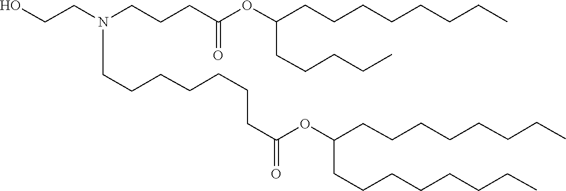 [SMILES: CCCCCCCCC(CCCCCCCC)OC(=O)CCCCCCCN(CCO)CCCC(=O)OC(CCCCC)CCCCCCCC] |
| I 72 | 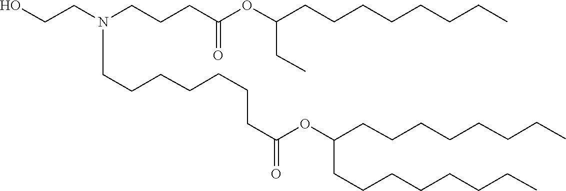 [SMILES: CCCCCCCCC(CC)OC(=O)CCCN(CCO)CCCCCCCC(=O)OC(CCCCCCCC)CCCCCCCC] |
| I 73 | 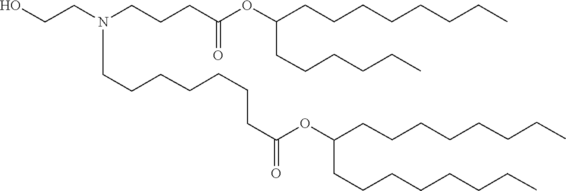 [SMILES: CCCCCCCCC(CCCCCCCC)OC(=O)CCCCCCCN(CCO)CCCC(=O)OC(CCCCCC)CCCCCCCC] |
| I 74 | 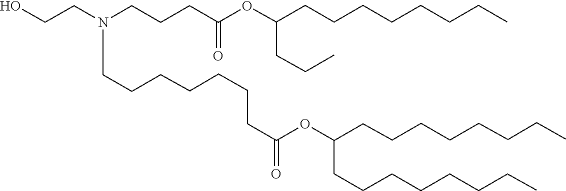 [SMILES: CCCCCCCCC(CCC)OC(=O)CCCN(CCO)CCCCCCCC(=O)OC(CCCCCCCC)CCCCCCCC] |
| I 75 | 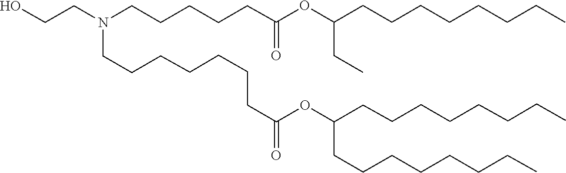 [SMILES: CCCCCCCCC(CC)OC(=O)CCCCCN(CCO)CCCCCCCC(=O)OC(CCCCCCCC)CCCCCCCC] |
| I 76 | 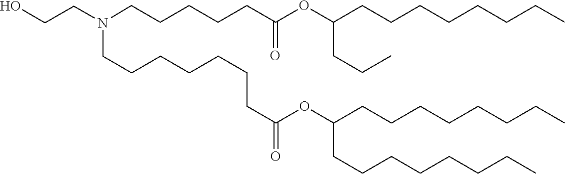 [SMILES: CCCCCCCCC(CCC)OC(=O)CCCCCN(CCO)CCCCCCCC(=O)OC(CCCCCCCC)CCCCCCCC] |
| I 77 | 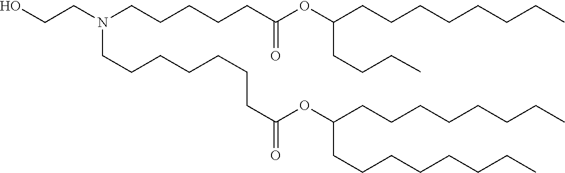 [SMILES: CCCCCCCCC(CCCC)OC(=O)CCCCCN(CCO)CCCCCCCC(=O)OC(CCCCCCCC)CCCCCCCC] |
| I 78 | 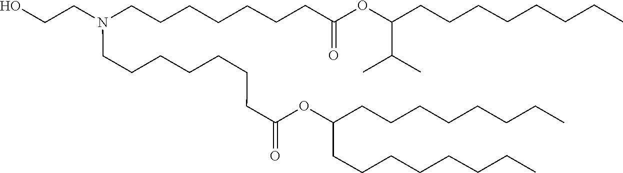 [SMILES: CCCCCCCCC(CCCCCCCC)OC(=O)CCCCCCCN(CCO)CCCCCCCC(=O)OC(CCCCCCCC)C(C)C] |
| I 79 | 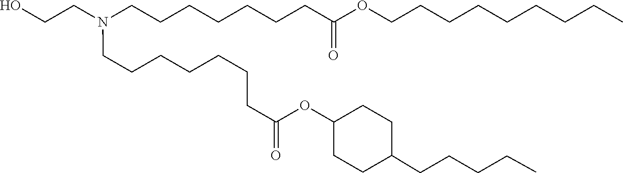 [SMILES: CCCCCCCCCOC(=O)CCCCCCCN(CCO)CCCCCCCC(=O)OC1CCC(CCCCC)CC1] |
| I 80 | 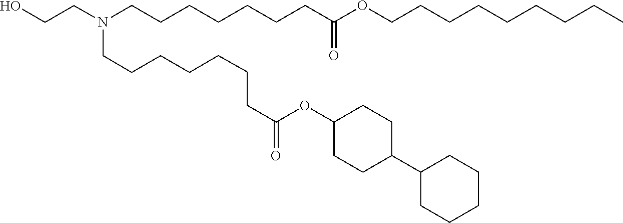 [SMILES: CCCCCCCCCOC(=O)CCCCCCCN(CCO)CCCCCCCC(=O)OC1CCC(C2CCCCC2)CC1] |
| I 81 | 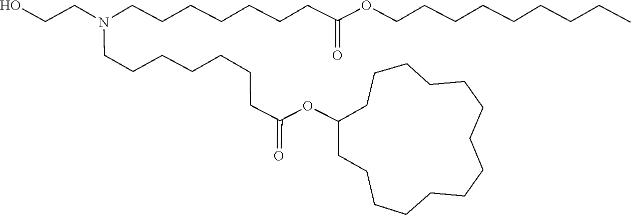 [SMILES: CCCCCCCCCOC(=O)CCCCCCCN(CCO)CCCCCCCC(=O)OC1CCCCCCCCCCCCCC1] |
| I 82 | 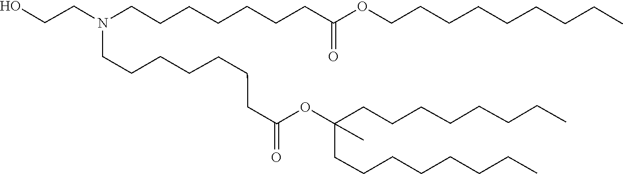 [SMILES: CCCCCCCCCOC(=O)CCCCCCCN(CCO)CCCCCCCC(=O)OC(C)(CCCCCCCC)CCCCCCCC] |
| I 83 | 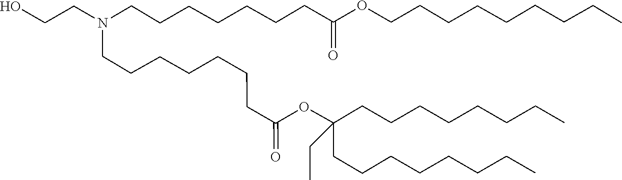 [SMILES: CCCCCCCCCOC(=O)CCCCCCCN(CCO)CCCCCCCC(=O)OC(CC)(CCCCCCCC)CCCCCCCC] |
| I 84 | 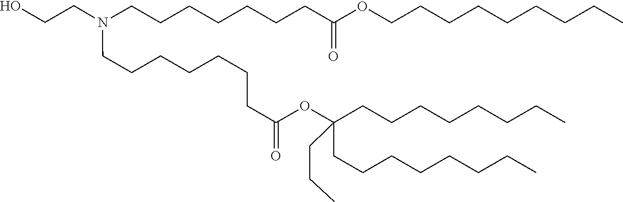 [SMILES: CCCCCCCCCOC(=O)CCCCCCCN(CCO)CCCCCCCC(=O)OC(CCC)(CCCCCCCC)CCCCCCCC] |
| I 85 | 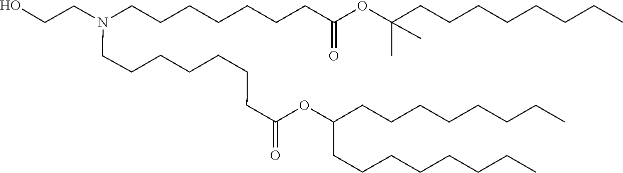 [SMILES: CCCCCCCCC(CCCCCCCC)OC(=O)CCCCCCCN(CCO)CCCCCCCC(=O)OC(C)(C)CCCCCCCC] |
| I 86 | 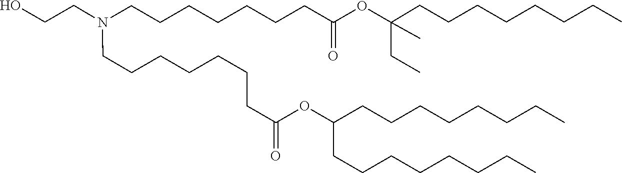 [SMILES: CCCCCCCCC(CCCCCCCC)OC(=O)CCCCCCCN(CCO)CCCCCCCC(=O)OC(C)(CC)CCCCCCCC] |
| I 87 | 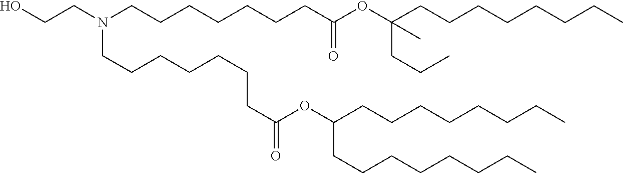 [SMILES: CCCCCCCCC(CCCCCCCC)OC(=O)CCCCCCCN(CCO)CCCCCCCC(=O)OC(C)(CCC)CCCCCCCC] |
| I 88 | 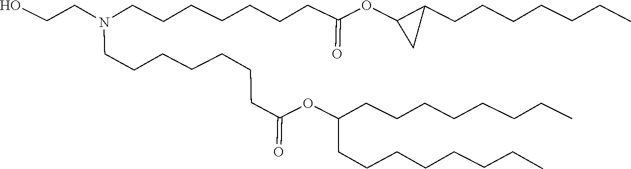 [SMILES: CCCCCCCCC(CCCCCCCC)OC(=O)CCCCCCCN(CCO)CCCCCCCC(=O)OC1CC1CCCCCCC] |
| I 89 | 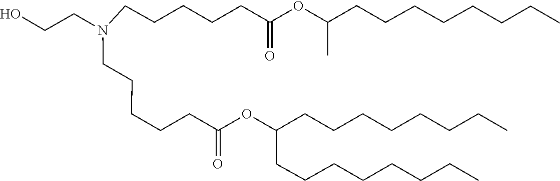 [SMILES: CCCCCCCCC(C)OC(=O)CCCCCN(CCO)CCCCCC(=O)OC(CCCCCCCC)CCCCCCCC] |
| I 90 | 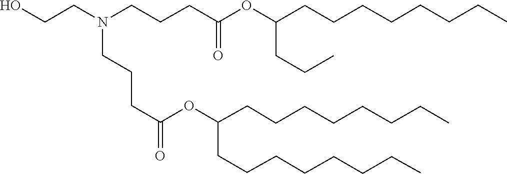 [SMILES: CCCCCCCCC(CCC)OC(=O)CCCN(CCO)CCCC(=O)OC(CCCCCCCC)CCCCCCCC] |
| I 91 | 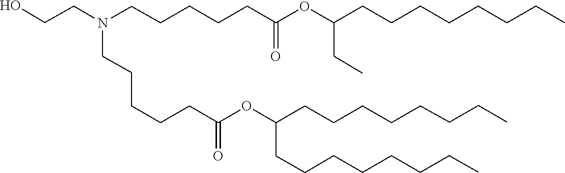 [SMILES: CCCCCCCCC(CC)OC(=O)CCCCCN(CCO)CCCCCC(=O)OC(CCCCCCCC)CCCCCCCC] |
| I 92 | 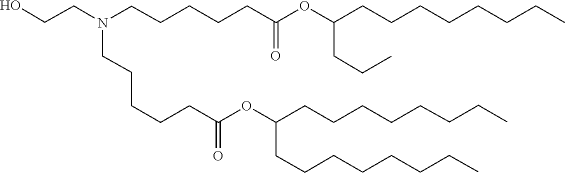 [SMILES: CCCCCCCCC(CCC)OC(=O)CCCCCN(CCO)CCCCCC(=O)OC(CCCCCCCC)CCCCCCCC] |
| I 93 | 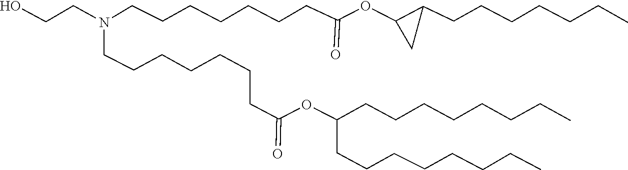 [SMILES: CCCCCCCCC(CCCCCCCC)OC(=O)CCCCCCCN(CCO)CCCCCCCC(=O)OC1CC1CCCCCCC] |
| I 94 | 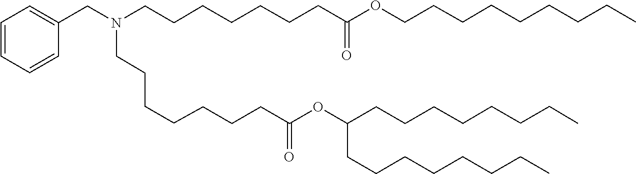 [SMILES: CCCCCCCCCOC(=O)CCCCCCCN(CCCCCCCC(=O)OC(CCCCCCCC)CCCCCCCC)Cc1ccccc1] |
| I 95 | 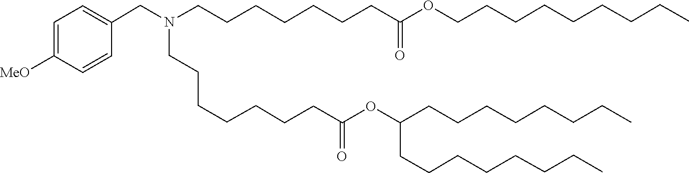 [SMILES: CCCCCCCCCOC(=O)CCCCCCCN(CCCCCCCC(=O)OC(CCCCCCCC)CCCCCCCC)Cc1ccc(OC)cc1] |
| I 96 | 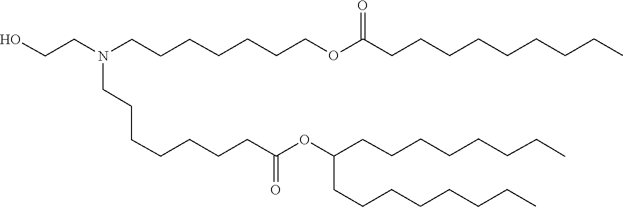 [SMILES: CCCCCCCCCC(=O)OCCCCCCCN(CCO)CCCCCCCC(=O)OC(CCCCCCCC)CCCCCCCC] |
| I 97 | 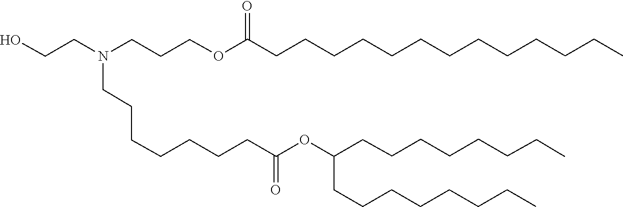 [SMILES: CCCCCCCCCCCCCC(=O)OCCCN(CCO)CCCCCCCC(=O)OC(CCCCCCCC)CCCCCCCC] |
| I 98 | 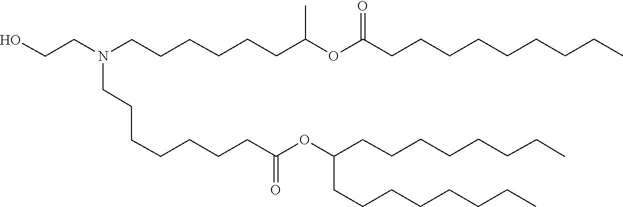 [SMILES: CCCCCCCCCC(=O)OC(C)CCCCCCN(CCO)CCCCCCCC(=O)OC(CCCCCCCC)CCCCCCCC] |
| I 99 | 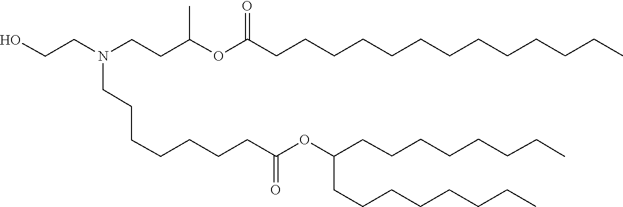 [SMILES: CCCCCCCCCCCCCC(=O)OC(C)CCN(CCO)CCCCCCCC(=O)OC(CCCCCCCC)CCCCCCCC] |
| I 100 | 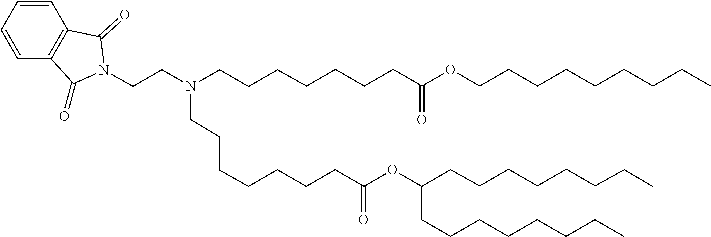 [SMILES: CCCCCCCCCOC(=O)CCCCCCCN(CCCCCCCC(=O)OC(CCCCCCCC)CCCCCCCC)CCN1C(=O)c2ccccc2C1=O] |
| I 10I1 | 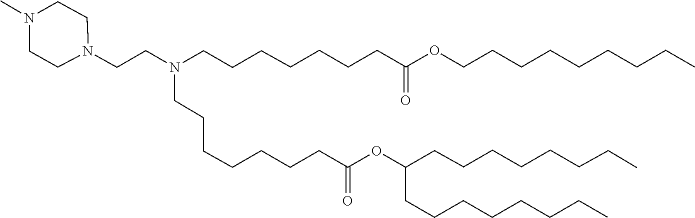 [SMILES: CCCCCCCCCOC(=O)CCCCCCCN(CCCCCCCC(=O)OC(CCCCCCCC)CCCCCCCC)CCN1CCN(C)CC1] |
| I 102 | 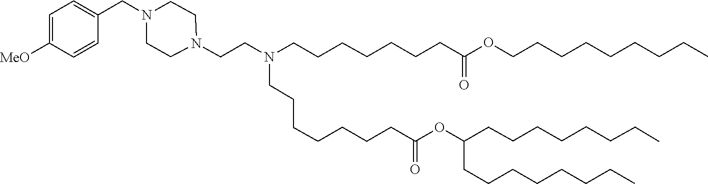 [SMILES: CCCCCCCCCOC(=O)CCCCCCCN(CCCCCCCC(=O)OC(CCCCCCCC)CCCCCCCC)CCN1CCN(Cc2ccc(OC)cc2)CC1] |
| I 103 | 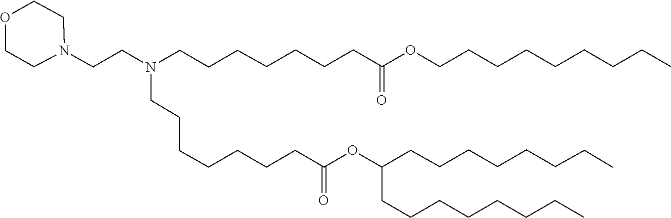 [SMILES: CCCCCCCCCOC(=O)CCCCCCCN(CCCCCCCC(=O)OC(CCCCCCCC)CCCCCCCC)CCN1CCOCC1] |
| I 104 | 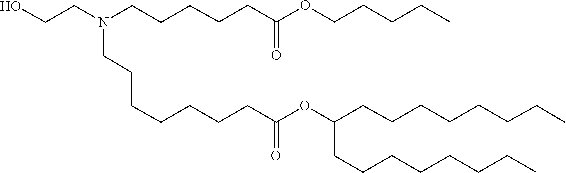 [SMILES: CCCCCCCCC(CCCCCCCC)OC(=O)CCCCCCCN(CCO)CCCCCC(=O)OCCCCC] |
| I 105 | 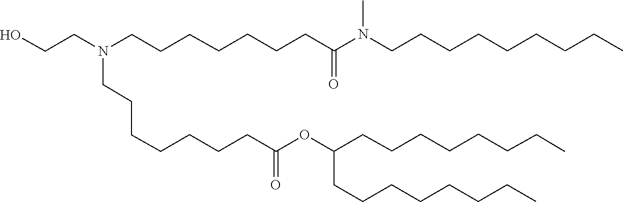 [SMILES: CCCCCCCCCN(C)C(=O)CCCCCCCN(CCO)CCCCCCCC(=O)OC(CCCCCCCC)CCCCCCCC] |
| I 106 | 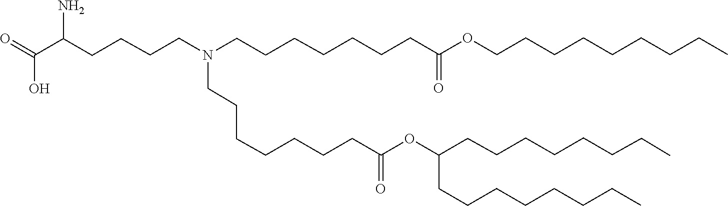 [SMILES: CCCCCCCCCOC(=O)CCCCCCCN(CCCCCCCC(=O)OC(CCCCCCCC)CCCCCCCC)CCCCC(N)C(=O)O] |
| I 107 | 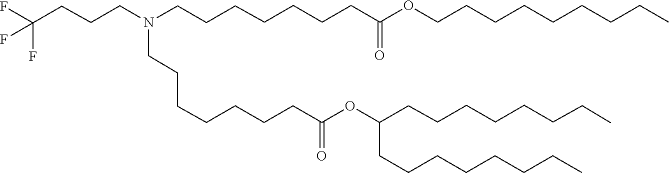 [SMILES: CCCCCCCCCOC(=O)CCCCCCCN(CCCCCCCC(=O)OC(CCCCCCCC)CCCCCCCC)CCCC(F)(F)F] |
| I 108 | 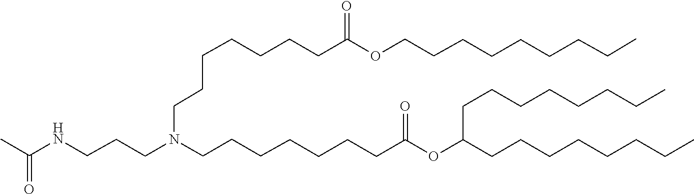 [SMILES: CCCCCCCCCOC(=O)CCCCCCCN(CCCCCCCC(=O)OC(CCCCCCCC)CCCCCCCC)CCCNC(C)=O] |
| I 109 | 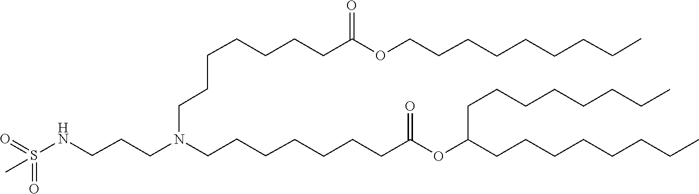 [SMILES: CCCCCCCCCOC(=O)CCCCCCCN(CCCCCCCC(=O)OC(CCCCCCCC)CCCCCCCC)CCCNS(C)(=O)=O] |
| I 110 | 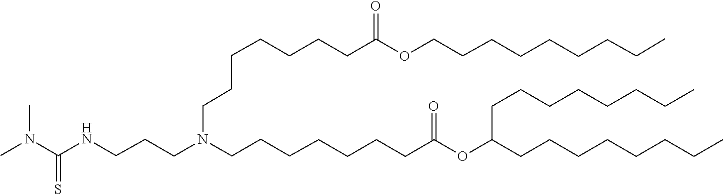 [SMILES: CCCCCCCCCOC(=O)CCCCCCCN(CCCCCCCC(=O)OC(CCCCCCCC)CCCCCCCC)CCCNC(=S)N(C)C] |
| I 111 | 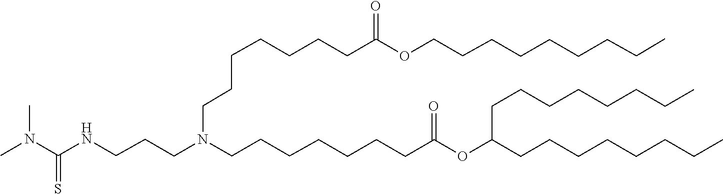 [SMILES: CCCCCCCCCOC(=O)CCCCCCCN(CCCCCCCC(=O)OC(CCCCCCCC)CCCCCCCC)CCCNC(=S)N(C)C] |
| I 112 | 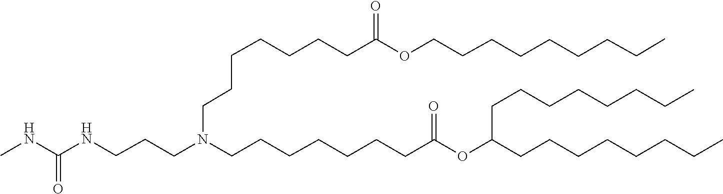 [SMILES: CCCCCCCCCOC(=O)CCCCCCCN(CCCCCCCC(=O)OC(CCCCCCCC)CCCCCCCC)CCCNC(=O)NC] |
| I 113 | 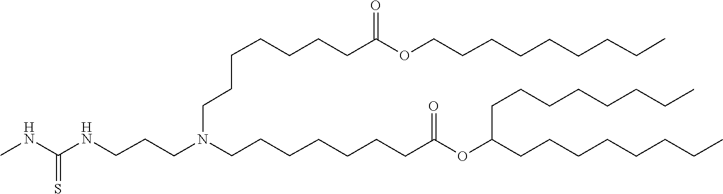 [SMILES: CCCCCCCCCOC(=O)CCCCCCCN(CCCCCCCC(=O)OC(CCCCCCCC)CCCCCCCC)CCCNC(=S)NC] |
| I 114 | 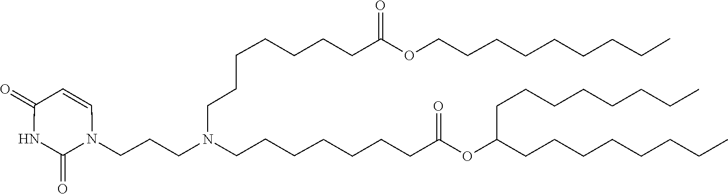 [SMILES: CCCCCCCCCOC(=O)CCCCCCCN(CCCCCCCC(=O)OC(CCCCCCCC)CCCCCCCC)CCCn1ccc(=O)[nH]c1=O] |
| I 115 | 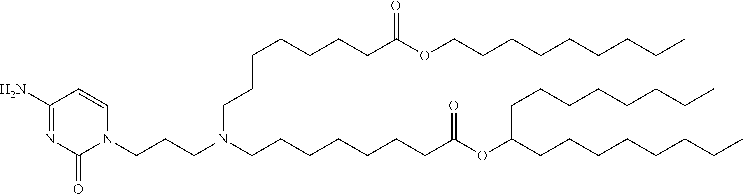 [SMILES: CCCCCCCCCOC(=O)CCCCCCCN(CCCCCCCC(=O)OC(CCCCCCCC)CCCCCCCC)CCCn1ccc(N)nc1=O] |
| I 116 | 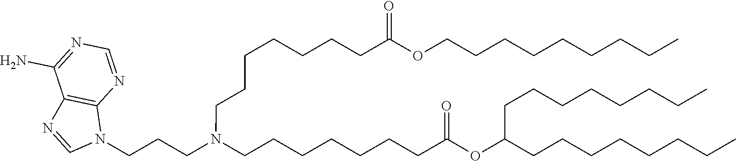 [SMILES: CCCCCCCCCOC(=O)CCCCCCCN(CCCCCCCC(=O)OC(CCCCCCCC)CCCCCCCC)CCCn1cnc2c(N)ncnc21] |
| I 117 | 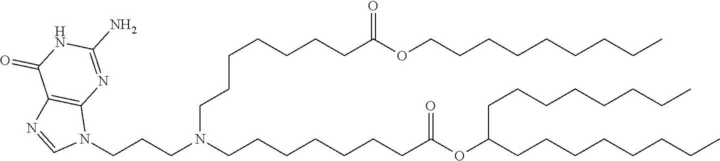 [SMILES: CCCCCCCCCOC(=O)CCCCCCCN(CCCCCCCC(=O)OC(CCCCCCCC)CCCCCCCC)CCCn1cnc2c(=O)[nH]c(N)nc21] |
| I 118 | 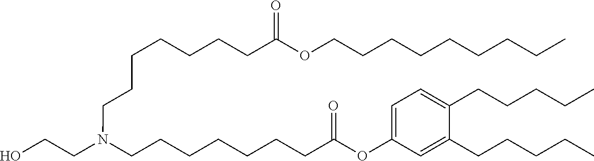 [SMILES: CCCCCCCCCOC(=O)CCCCCCCN(CCO)CCCCCCCC(=O)Oc1ccc(CCCCC)c(CCCCC)c1] |
| I 119 | 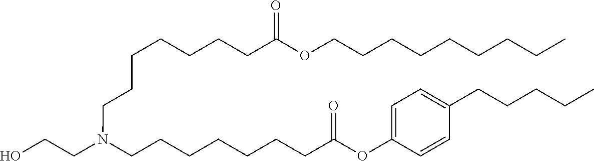 [SMILES: CCCCCCCCCOC(=O)CCCCCCCN(CCO)CCCCCCCC(=O)Oc1ccc(CCCCC)cc1] |
| I 120 | 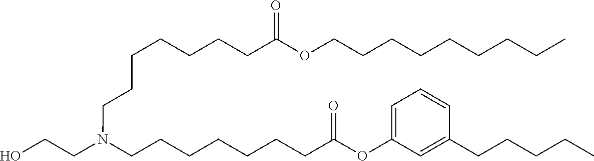 [SMILES: CCCCCCCCCOC(=O)CCCCCCCN(CCO)CCCCCCCC(=O)Oc1cccc(CCCCC)c1] |
| I 121 | 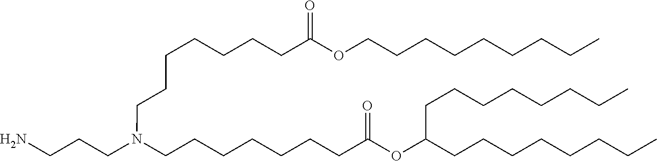 [SMILES: CCCCCCCCCOC(=O)CCCCCCCN(CCCN)CCCCCCCC(=O)OC(CCCCCCCC)CCCCCCCC] |
| I 122 | 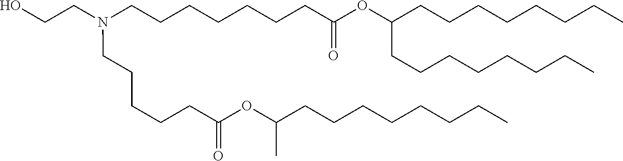 [SMILES: CCCCCCCCC(C)OC(=O)CCCCCN(CCO)CCCCCCCC(=O)OC(CCCCCCCC)CCCCCCCC] |
| I 123 | 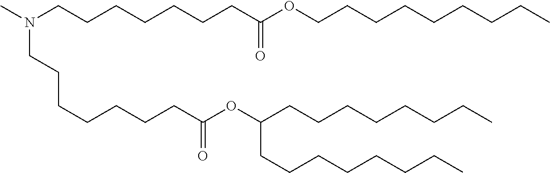 [SMILES: CCCCCCCCCOC(=O)CCCCCCCN(C)CCCCCCCC(=O)OC(CCCCCCCC)CCCCCCCC] |
| I 124 | 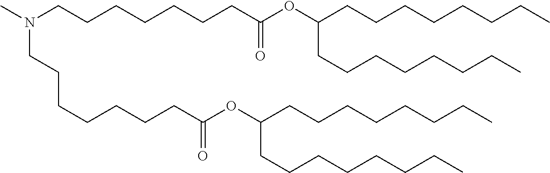 [SMILES: CCCCCCCCC(CCCCCCCC)OC(=O)CCCCCCCN(C)CCCCCCCC(=O)OC(CCCCCCCC)CCCCCCCC] |
| I 125 | 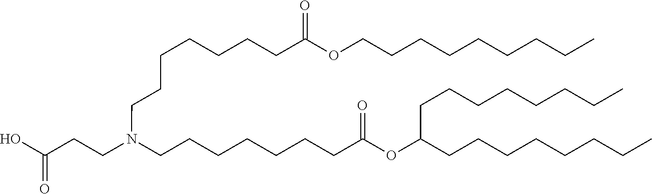 [SMILES: CCCCCCCCCOC(=O)CCCCCCCN(CCCCCCCC(=O)OC(CCCCCCCC)CCCCCCCC)CCC(=O)O] |
| I 126 | 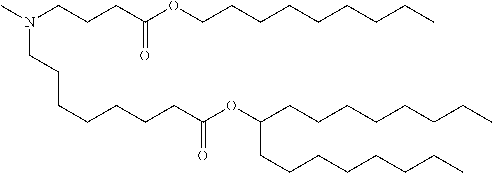 [SMILES: CCCCCCCCCOC(=O)CCCN(C)CCCCCCCC(=O)OC(CCCCCCCC)CCCCCCCC] |
| I 127 | 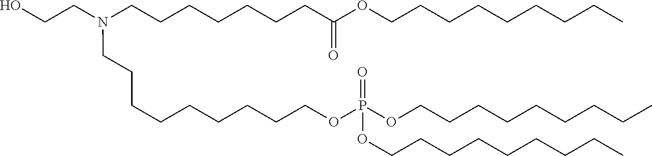 [SMILES: CCCCCCCCCOC(=O)CCCCCCCN(CCO)CCCCCCCCCOP(=O)(OCCCCCCCCC)OCCCCCCCCC] |
| I 128 | 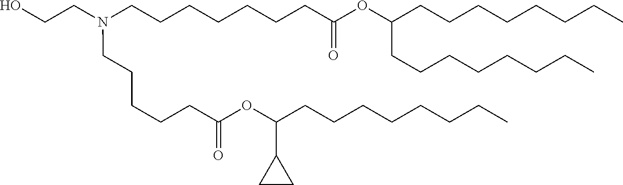 [SMILES: CCCCCCCCC(CCCCCCCC)OC(=O)CCCCCCCN(CCO)CCCCCC(=O)OC(CCCCCCCC)C1CC1] |
| I 129 | 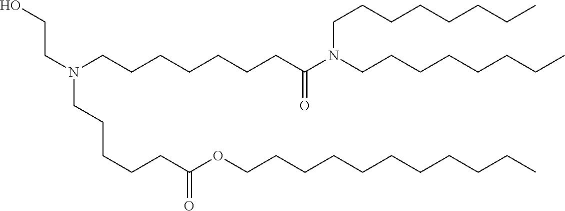 [SMILES: CCCCCCCCCCCOC(=O)CCCCCN(CCO)CCCCCCCC(=O)N(CCCCCCCC)CCCCCCCC] |
| I 130 | 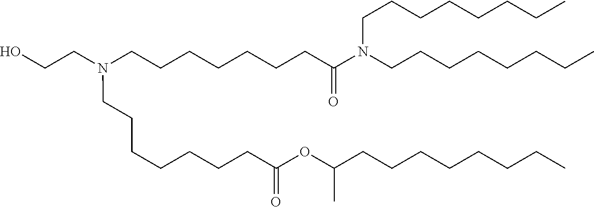 [SMILES: CCCCCCCCC(C)OC(=O)CCCCCCCN(CCO)CCCCCCCC(=O)N(CCCCCCCC)CCCCCCCC] |
| I 131 | 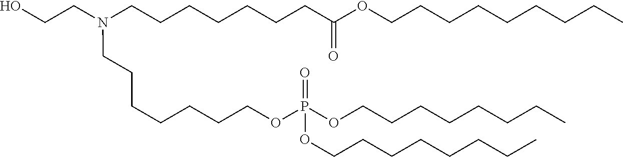 [SMILES: CCCCCCCCCOC(=O)CCCCCCCN(CCO)CCCCCCCOP(=O)(OCCCCCCCC)OCCCCCCCC] |
| I 132 | 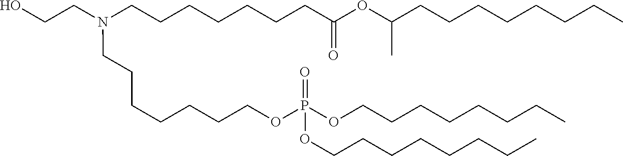 [SMILES: CCCCCCCCOP(=O)(OCCCCCCCC)OCCCCCCCN(CCO)CCCCCCCC(=O)OC(C)CCCCCCCC] |
| I 133 | 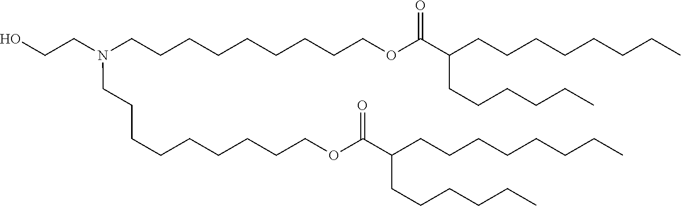 [SMILES: CCCCCCCCC(CCCCCC)C(=O)OCCCCCCCCCN(CCO)CCCCCCCCCOC(=O)C(CCCCCC)CCCCCCCC] |
| I 134 | 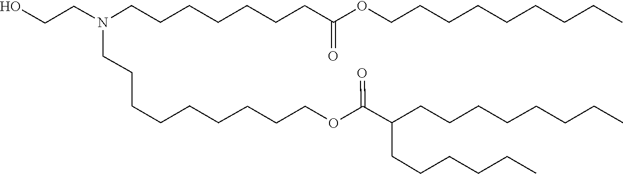 [SMILES: CCCCCCCCCOC(=O)CCCCCCCN(CCO)CCCCCCCCCOC(=O)C(CCCCCC)CCCCCCCC] |
| I 135 | 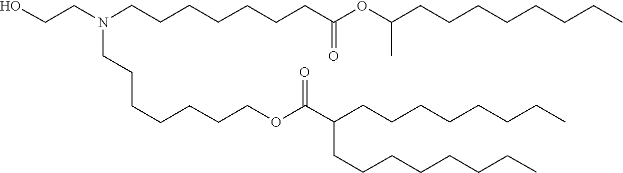 [SMILES: CCCCCCCCC(C)OC(=O)CCCCCCCN(CCO)CCCCCCCOC(=O)C(CCCCCCCC)CCCCCCCC] |
| I 136 | 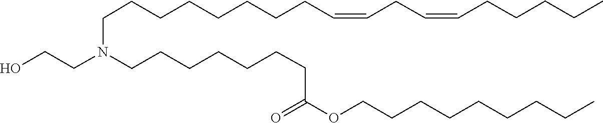 [SMILES: CCCCC/C=C\C/C=C\CCCCCCCCN(CCO)CCCCCCCC(=O)OCCCCCCCCC] |
| I 137 | 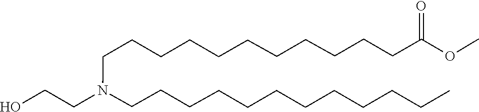 [SMILES: CCCCCCCCCCCCN(CCO)CCCCCCCCCCCC(=O)OC] |
| I 138 | 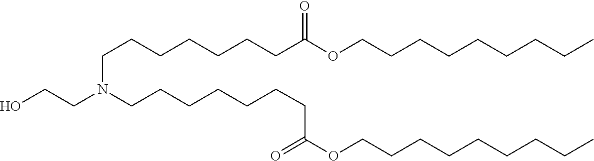 [SMILES: CCCCCCCCCOC(=O)CCCCCCCN(CCO)CCCCCCCC(=O)OCCCCCCCCC] |
| I 139 | 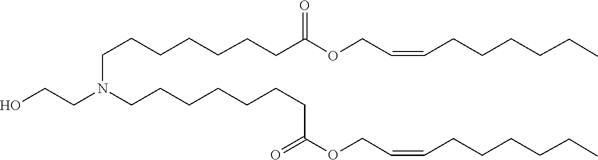 [SMILES: CCCCCC/C=C\COC(=O)CCCCCCCN(CCO)CCCCCCCC(=O)OC/C=C\CCCCCC] |
| I 140 | 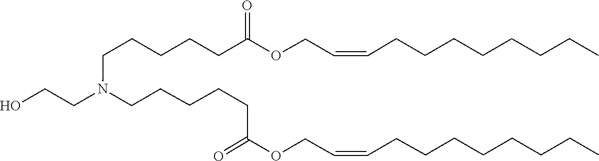 [SMILES: CCCCCCCC/C=C\COC(=O)CCCCCN(CCO)CCCCCC(=O)OC/C=C\CCCCCCCC] |
| I 141 | 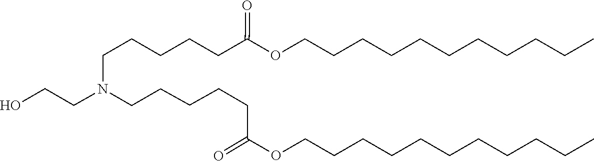 [SMILES: CCCCCCCCCCCOC(=O)CCCCCN(CCO)CCCCCC(=O)OCCCCCCCCCCC] |
| I 142 | 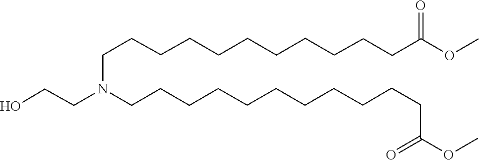 [SMILES: COC(=O)CCCCCCCCCCCN(CCO)CCCCCCCCCCCC(=O)OC] |
| I 143 | 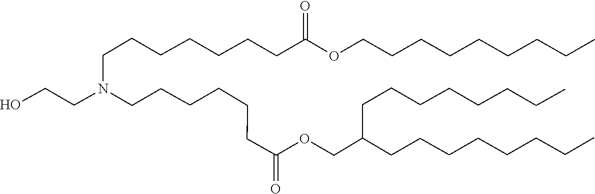 [SMILES: CCCCCCCCCOC(=O)CCCCCCCN(CCO)CCCCCCC(=O)OCC(CCCCCCCC)CCCCCCCC] |
| I 144 | 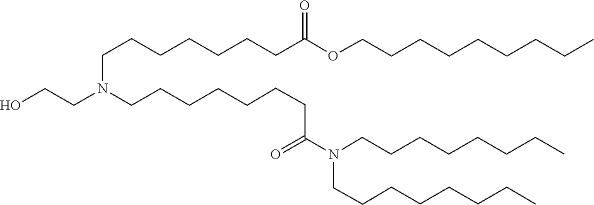 [SMILES: CCCCCCCCCOC(=O)CCCCCCCN(CCO)CCCCCCCC(=O)N(CCCCCCCC)CCCCCCCC] |
| I 145 | 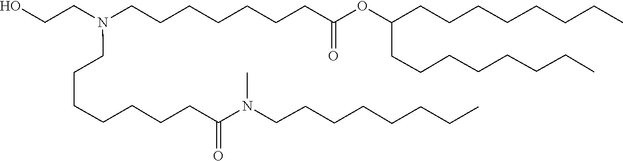 [SMILES: CCCCCCCCC(CCCCCCCC)OC(=O)CCCCCCCN(CCO)CCCCCCCC(=O)N(C)CCCCCCCC] |
| I 146 | 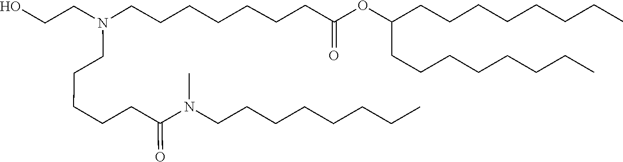 [SMILES: CCCCCCCCC(CCCCCCCC)OC(=O)CCCCCCCN(CCO)CCCCCC(=O)N(C)CCCCCCCC] |
| I 147 | 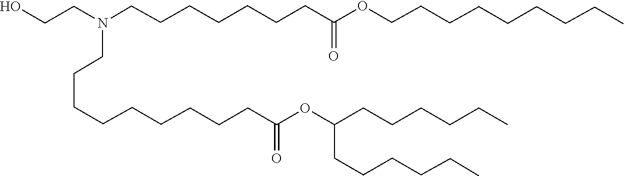 [SMILES: CCCCCCCCCOC(=O)CCCCCCCN(CCO)CCCCCCCCCC(=O)OC(CCCCCC)CCCCCC] |
| I 148 | 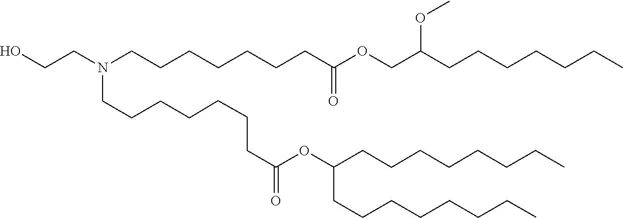 [SMILES: CCCCCCCCC(CCCCCCCC)OC(=O)CCCCCCCN(CCO)CCCCCCCC(=O)OCC(CCCCCCC)OC] |
| I 149 | 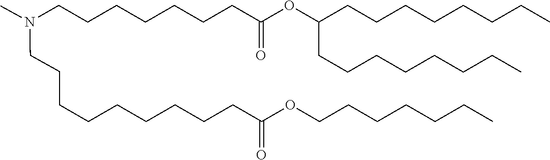 [SMILES: CCCCCCCCC(CCCCCCCC)OC(=O)CCCCCCCN(C)CCCCCCCCCC(=O)OCCCCCCC] |
| I 150 | 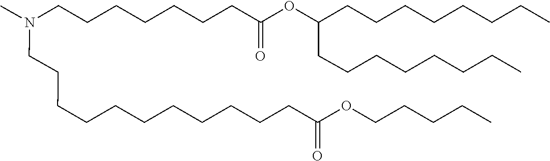 [SMILES: CCCCCCCCC(CCCCCCCC)OC(=O)CCCCCCCN(C)CCCCCCCCCCCC(=O)OCCCCC] |
| I 151 | 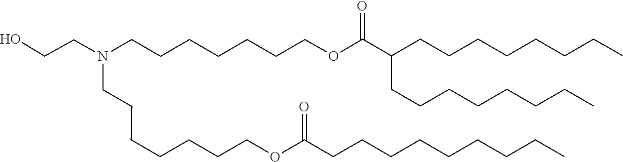 [SMILES: CCCCCCCCCC(=O)OCCCCCCCN(CCO)CCCCCCCOC(=O)C(CCCCCCCC)CCCCCCCC] |
| I 152 | 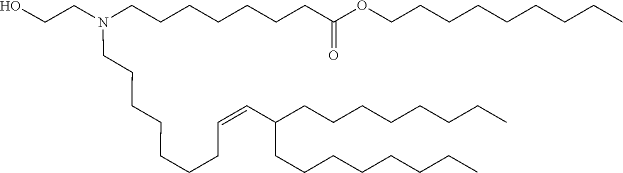 [SMILES: CCCCCCCCCOC(=O)CCCCCCCN(CCO)CCCCCCC/C=C\C(CCCCCCCC)CCCCCCCC] |
| I 153 | 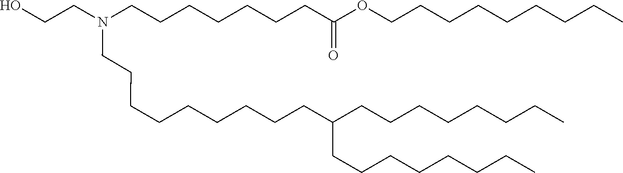 [SMILES: CCCCCCCCCOC(=O)CCCCCCCN(CCO)CCCCCCCCCC(CCCCCCCC)CCCCCCCC] |
| I 154 | 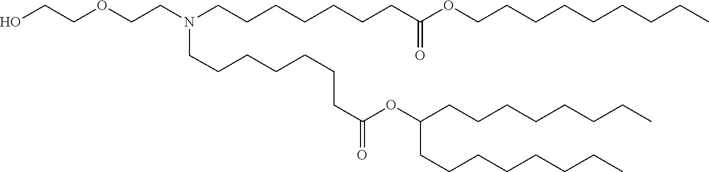 [SMILES: CCCCCCCCCOC(=O)CCCCCCCN(CCCCCCCC(=O)OC(CCCCCCCC)CCCCCCCC)CCOCCO] |
| I 155 | 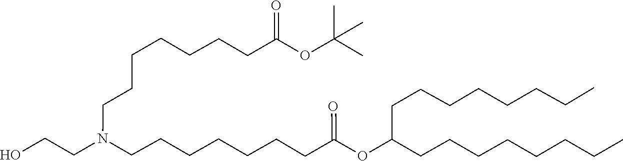 [SMILES: CCCCCCCCC(CCCCCCCC)OC(=O)CCCCCCCN(CCO)CCCCCCCC(=O)OC(C)(C)C] |
| I 156 | 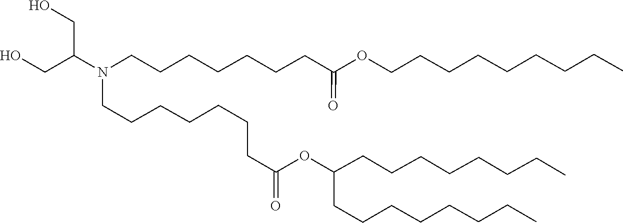 [SMILES: CCCCCCCCCOC(=O)CCCCCCCN(CCCCCCCC(=O)OC(CCCCCCCC)CCCCCCCC)C(CO)CO] |
| I 157 | 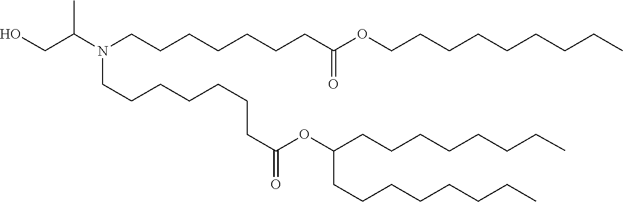 [SMILES: CCCCCCCCCOC(=O)CCCCCCCN(CCCCCCCC(=O)OC(CCCCCCCC)CCCCCCCC)C(C)CO] |
| I 158 | 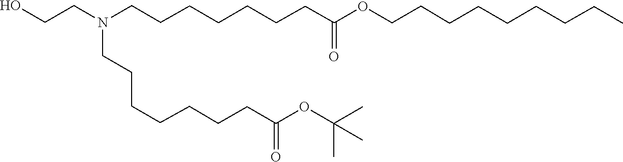 [SMILES: CCCCCCCCCOC(=O)CCCCCCCN(CCO)CCCCCCCC(=O)OC(C)(C)C] |
| I 159 | 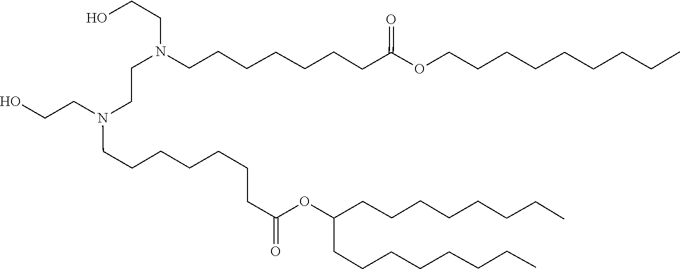 [SMILES: CCCCCCCCCOC(=O)CCCCCCCN(CCO)CCN(CCO)CCCCCCCC(=O)OC(CCCCCCCC)CCCCCCCC] |
| I 160 | 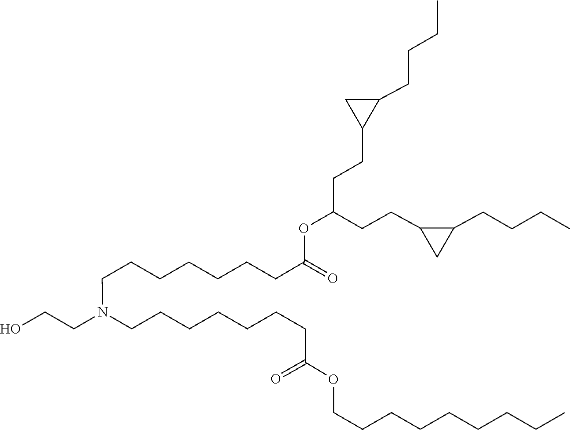 [SMILES: CCCCCCCCCOC(=O)CCCCCCCN(CCO)CCCCCCCC(=O)OC(CCC1CC1CCCC)CCC1CC1CCCC] |
| I 161 | 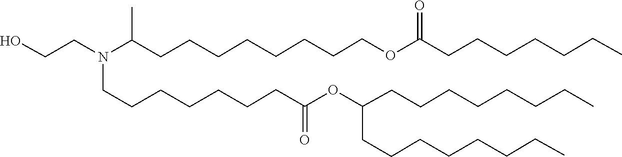 [SMILES: CCCCCCCCC(CCCCCCCC)OC(=O)CCCCCCCN(CCO)C(C)CCCCCCCCOC(=O)CCCCCCC] |
| I 162 | 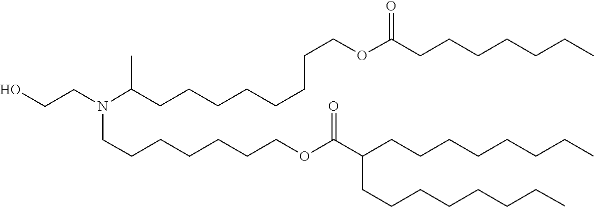 [SMILES: CCCCCCCCC(CCCCCCCC)C(=O)OCCCCCCCN(CCO)C(C)CCCCCCCCOC(=O)CCCCCCC] |
| I 163 | 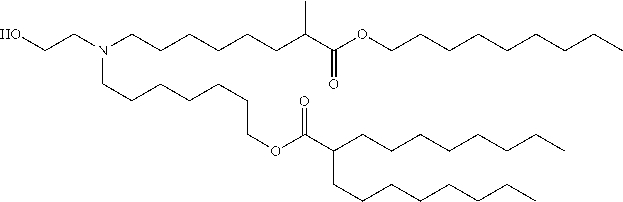 [SMILES: CCCCCCCCCOC(=O)C(C)CCCCCCN(CCO)CCCCCCCOC(=O)C(CCCCCCCC)CCCCCCCC] |
| I 164 | 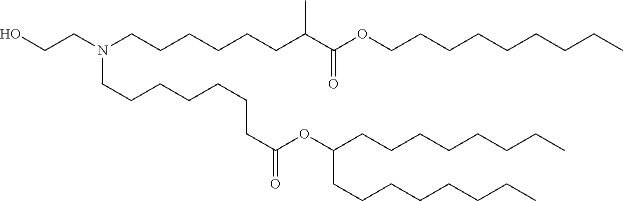 [SMILES: CCCCCCCCCOC(=O)C(C)CCCCCCN(CCO)CCCCCCCC(=O)OC(CCCCCCCC)CCCCCCCC] |
| I 165 | 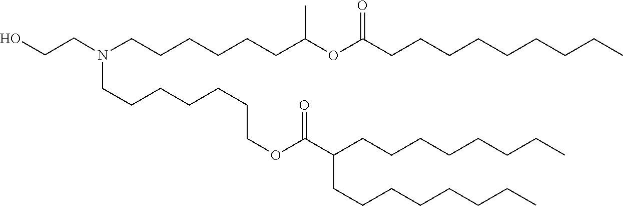 [SMILES: CCCCCCCCCC(=O)OC(C)CCCCCCN(CCO)CCCCCCCOC(=O)C(CCCCCCCC)CCCCCCCC] |
| I 166 | 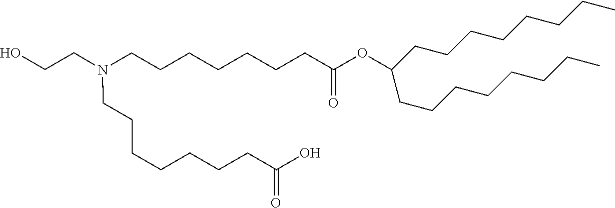 [SMILES: CCCCCCCCC(CCCCCCCC)OC(=O)CCCCCCCN(CCO)CCCCCCCC(=O)O] |
| I 167 | 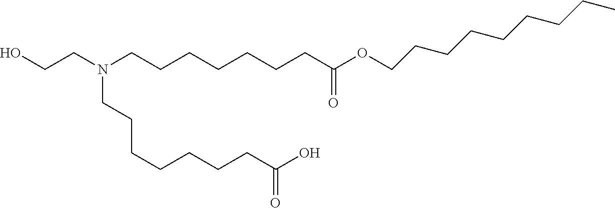 [SMILES: CCCCCCCCCOC(=O)CCCCCCCN(CCO)CCCCCCCC(=O)O] |
| I 168 | 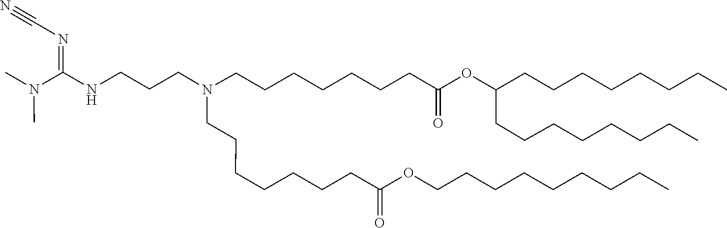 [SMILES: CCCCCCCCCOC(=O)CCCCCCCN(CCCCCCCC(=O)OC(CCCCCCCC)CCCCCCCC)CCCN/C(=N/C#N)N(C)C] |
| I 169 | 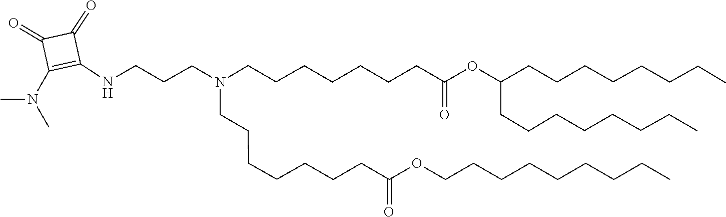 [SMILES: CCCCCCCCCOC(=O)CCCCCCCN(CCCCCCCC(=O)OC(CCCCCCCC)CCCCCCCC)CCCNc1c(N(C)C)c(=O)c1=O] |
| I 170 | 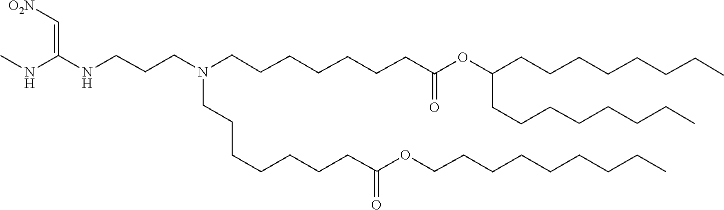 [SMILES: CCCCCCCCCOC(=O)CCCCCCCN(CCCCCCCC(=O)OC(CCCCCCCC)CCCCCCCC)CCCN/C(=C/[N+](=O)[O-])NC] |
| I 171 | 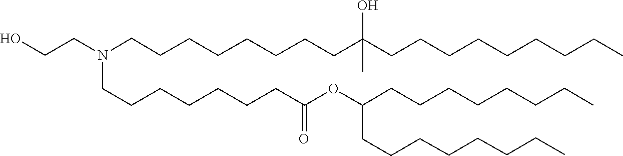 [SMILES: CCCCCCCCCC(C)(O)CCCCCCCCN(CCO)CCCCCCCC(=O)OC(CCCCCCCC)CCCCCCCC] |
| I 172 | 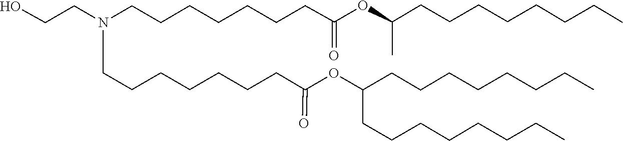 [SMILES: CCCCCCCCC(CCCCCCCC)OC(=O)CCCCCCCN(CCO)CCCCCCCC(=O)O[C@H](C)CCCCCCCC] |
| I 173 | 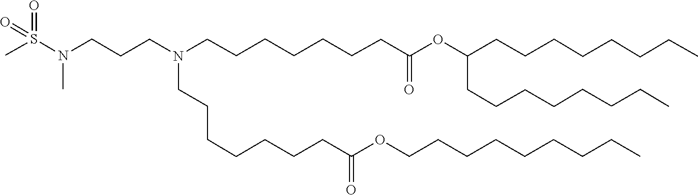 [SMILES: CCCCCCCCCOC(=O)CCCCCCCN(CCCCCCCC(=O)OC(CCCCCCCC)CCCCCCCC)CCCN(C)S(C)(=O)=O] |
| I 174 | 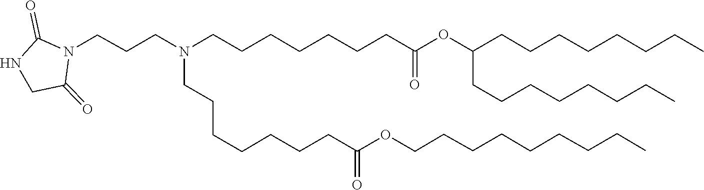 [SMILES: CCCCCCCCCOC(=O)CCCCCCCN(CCCCCCCC(=O)OC(CCCCCCCC)CCCCCCCC)CCCN1C(=O)CNC1=O] |
| I 175 | 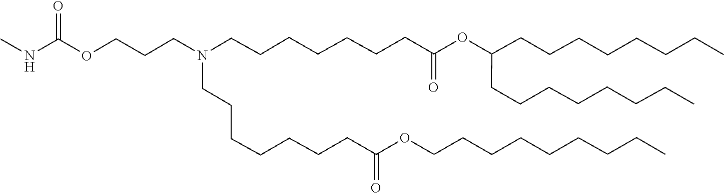 [SMILES: CCCCCCCCCOC(=O)CCCCCCCN(CCCCCCCC(=O)OC(CCCCCCCC)CCCCCCCC)CCCOC(=O)NC] |
| I 176 | 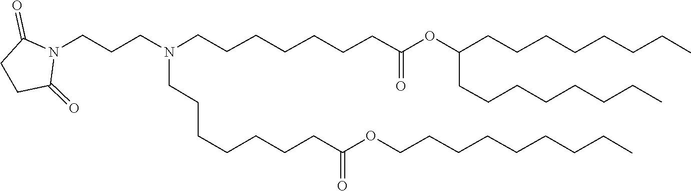 [SMILES: CCCCCCCCCOC(=O)CCCCCCCN(CCCCCCCC(=O)OC(CCCCCCCC)CCCCCCCC)CCCN1C(=O)CCC1=O] |
| I 177 | 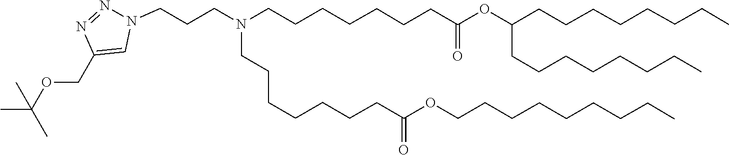 [SMILES: CCCCCCCCCOC(=O)CCCCCCCN(CCCCCCCC(=O)OC(CCCCCCCC)CCCCCCCC)CCCn1cc(COC(C)(C)C)nn1] |
| I 178 | 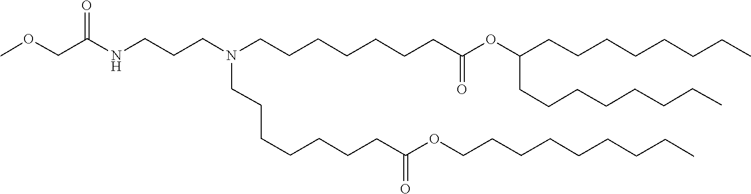 [SMILES: CCCCCCCCCOC(=O)CCCCCCCN(CCCCCCCC(=O)OC(CCCCCCCC)CCCCCCCC)CCCNC(=O)COC] |
| I 179 | 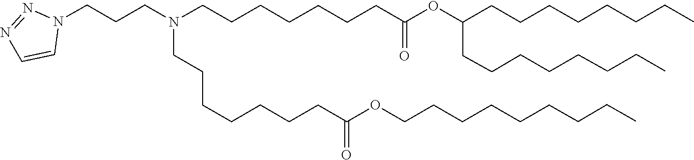 [SMILES: CCCCCCCCCOC(=O)CCCCCCCN(CCCCCCCC(=O)OC(CCCCCCCC)CCCCCCCC)CCCn1ccnn1] |
| I 115 | 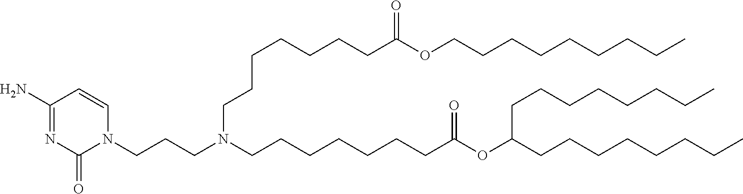 [SMILES: CCCCCCCCCOC(=O)CCCCCCCN(CCCCCCCC(=O)OC(CCCCCCCC)CCCCCCCC)CCCn1ccc(N)nc1=O] |
| I 116 | 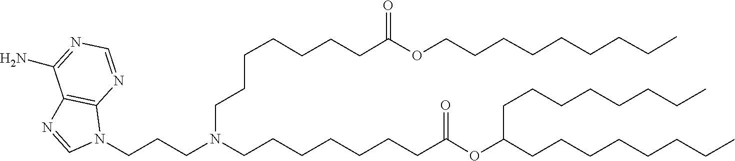 [SMILES: CCCCCCCCCOC(=O)CCCCCCCN(CCCCCCCC(=O)OC(CCCCCCCC)CCCCCCCC)CCCn1cnc2c(N)ncnc21] |
| I 117 | 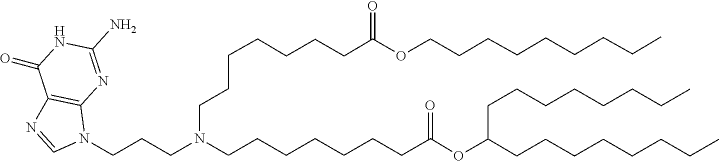 [SMILES: CCCCCCCCCOC(=O)CCCCCCCN(CCCCCCCC(=O)OC(CCCCCCCC)CCCCCCCC)CCCn1cnc2c(=O)[nH]c(N)nc21] |
| I 118 | 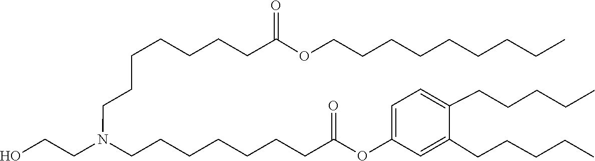 [SMILES: CCCCCCCCCOC(=O)CCCCCCCN(CCO)CCCCCCCC(=O)Oc1ccc(CCCCC)c(CCCCC)c1] |
| I 119 | 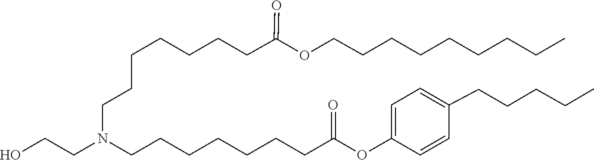 [SMILES: CCCCCCCCCOC(=O)CCCCCCCN(CCO)CCCCCCCC(=O)Oc1ccc(CCCCC)cc1] |
| I 120 | 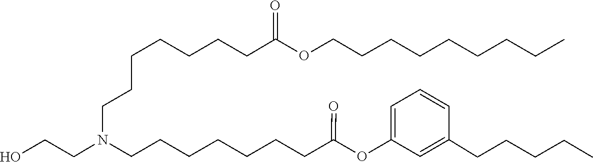 [SMILES: CCCCCCCCCOC(=O)CCCCCCCN(CCO)CCCCCCCC(=O)Oc1cccc(CCCCC)c1] |
| I 121 | 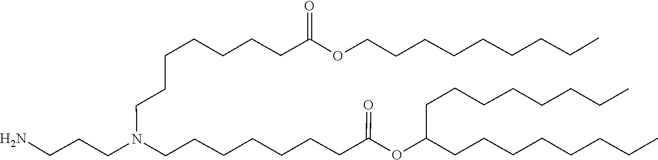 [SMILES: CCCCCCCCCOC(=O)CCCCCCCN(CCCN)CCCCCCCC(=O)OC(CCCCCCCC)CCCCCCCC] |
| I 122 | 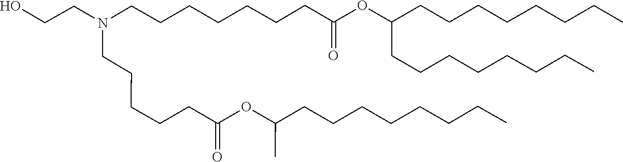 [SMILES: CCCCCCCCC(C)OC(=O)CCCCCN(CCO)CCCCCCCC(=O)OC(CCCCCCCC)CCCCCCCC] |
| I 123 | 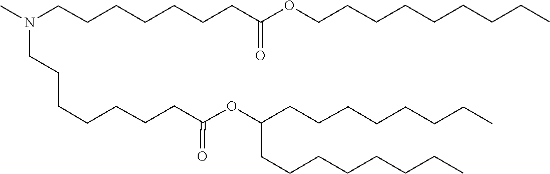 [SMILES: CCCCCCCCCOC(=O)CCCCCCCN(C)CCCCCCCC(=O)OC(CCCCCCCC)CCCCCCCC] |
| I 124 | 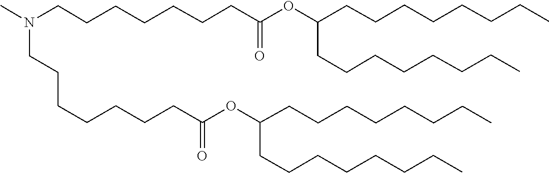 [SMILES: CCCCCCCCC(CCCCCCCC)OC(=O)CCCCCCCN(C)CCCCCCCC(=O)OC(CCCCCCCC)CCCCCCCC] |
| I 125 | 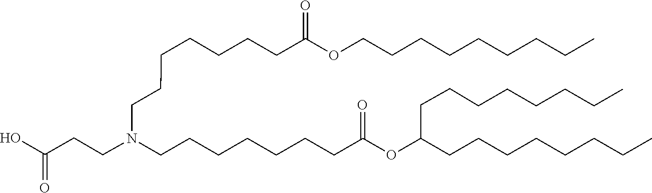 [SMILES: CCCCCCCCCOC(=O)CCCCCCCN(CCCCCCCC(=O)OC(CCCCCCCC)CCCCCCCC)CCC(=O)O] |
| I 126 | 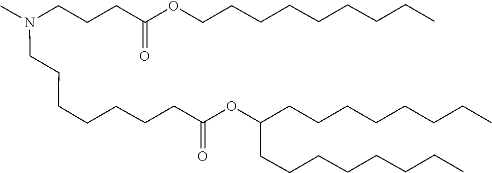 [SMILES: CCCCCCCCCOC(=O)CCCN(C)CCCCCCCC(=O)OC(CCCCCCCC)CCCCCCCC] |
| I 127 | 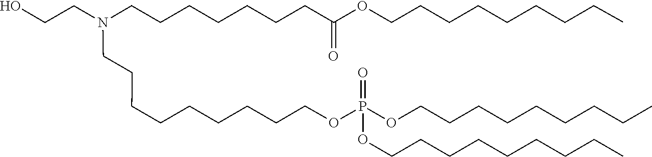 [SMILES: CCCCCCCCCOC(=O)CCCCCCCN(CCO)CCCCCCCCCOP(=O)(OCCCCCCCCC)OCCCCCCCCC] |
| I 128 | 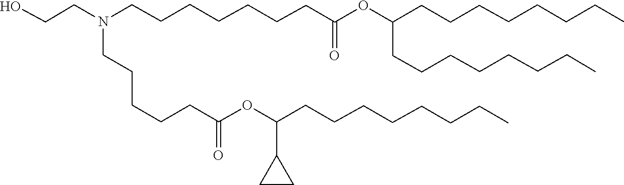 [SMILES: CCCCCCCCC(CCCCCCCC)OC(=O)CCCCCCCN(CCO)CCCCCC(=O)OC(CCCCCCCC)C1CC1] |
| I 129 | 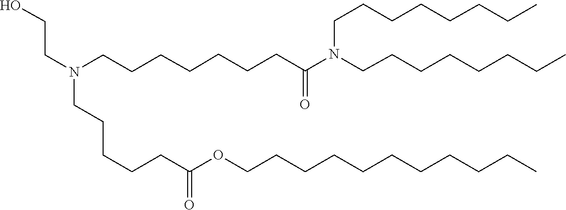 [SMILES: CCCCCCCCCCCOC(=O)CCCCCN(CCO)CCCCCCCC(=O)N(CCCCCCCC)CCCCCCCC] |
| I 130 | 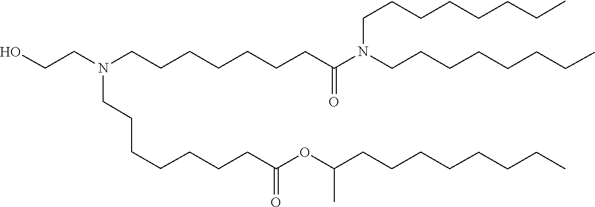 [SMILES: CCCCCCCCC(C)OC(=O)CCCCCCCN(CCO)CCCCCCCC(=O)N(CCCCCCCC)CCCCCCCC] |
| I 131 | 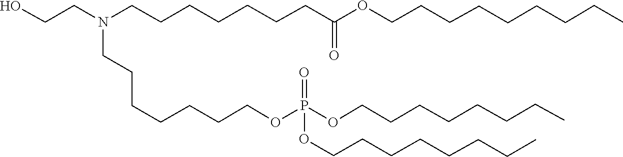 [SMILES: CCCCCCCCCOC(=O)CCCCCCCN(CCO)CCCCCCCOP(=O)(OCCCCCCCC)OCCCCCCCC] |
| I 132 | 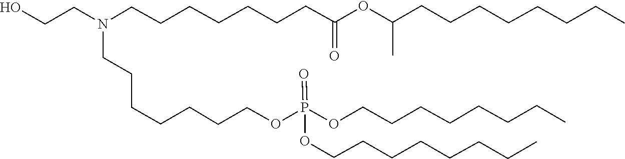 [SMILES: CCCCCCCCOP(=O)(OCCCCCCCC)OCCCCCCCN(CCO)CCCCCCCC(=O)OC(C)CCCCCCCC] |
| I 133 | 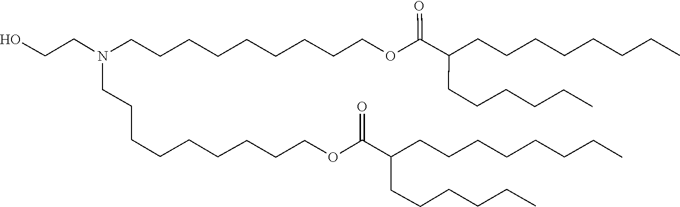 [SMILES: CCCCCCCCC(CCCCCC)C(=O)OCCCCCCCCCN(CCO)CCCCCCCCCOC(=O)C(CCCCCC)CCCCCCCC] |
| I 134 | 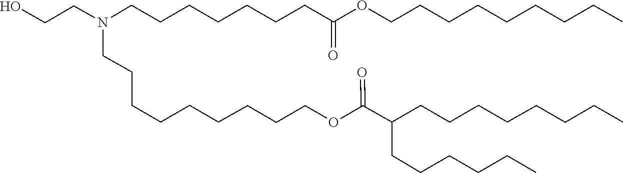 [SMILES: CCCCCCCCCOC(=O)CCCCCCCN(CCO)CCCCCCCCCOC(=O)C(CCCCCC)CCCCCCCC] |
| I 135 | 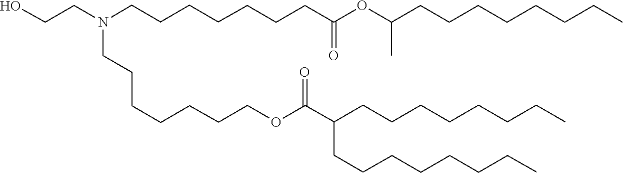 [SMILES: CCCCCCCCC(C)OC(=O)CCCCCCCN(CCO)CCCCCCCOC(=O)C(CCCCCCCC)CCCCCCCC] |
| I 136 | 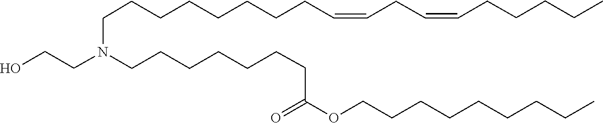 [SMILES: CCCCC/C=C\C/C=C\CCCCCCCCN(CCO)CCCCCCCC(=O)OCCCCCCCCC] |
| I 137 | 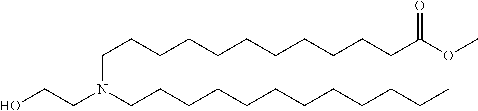 [SMILES: CCCCCCCCCCCCN(CCO)CCCCCCCCCCCC(=O)OC] |
| I 138 | 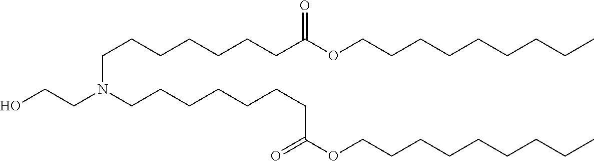 [SMILES: CCCCCCCCCOC(=O)CCCCCCCN(CCO)CCCCCCCC(=O)OCCCCCCCCC] |
| I 139 | 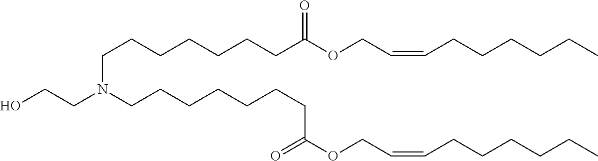 [SMILES: CCCCCC/C=C\COC(=O)CCCCCCCN(CCO)CCCCCCCC(=O)OC/C=C\CCCCCC] |
| I 140 | 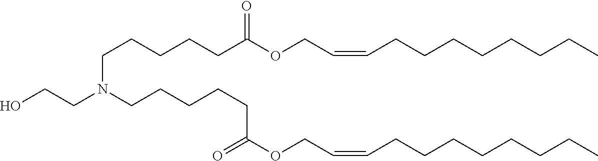 [SMILES: CCCCCCCC/C=C\COC(=O)CCCCCN(CCO)CCCCCC(=O)OC/C=C\CCCCCCCC] |
| I 141 | 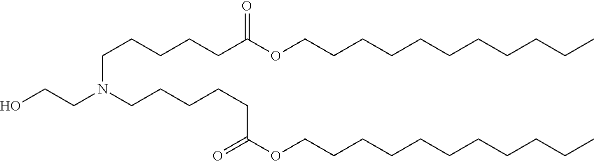 [SMILES: CCCCCCCCCCCOC(=O)CCCCCN(CCO)CCCCCC(=O)OCCCCCCCCCCC] |
| I 142 | 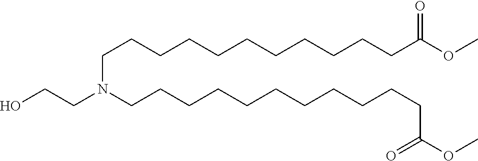 [SMILES: COC(=O)CCCCCCCCCCCN(CCO)CCCCCCCCCCCC(=O)OC] |
| I 143 | 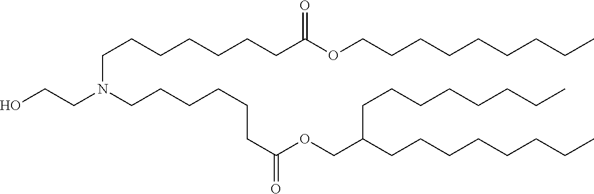 [SMILES: CCCCCCCCCOC(=O)CCCCCCCN(CCO)CCCCCCC(=O)OCC(CCCCCCCC)CCCCCCCC] |
| I 144 | 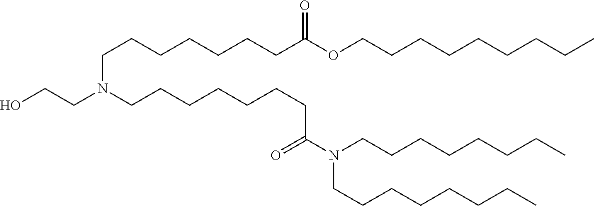 [SMILES: CCCCCCCCCOC(=O)CCCCCCCN(CCO)CCCCCCCC(=O)N(CCCCCCCC)CCCCCCCC] |
| I 145 | 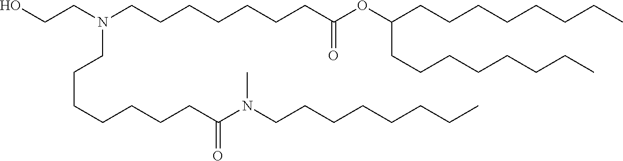 [SMILES: CCCCCCCCC(CCCCCCCC)OC(=O)CCCCCCCN(CCO)CCCCCCCC(=O)N(C)CCCCCCCC] |
| I 146 | 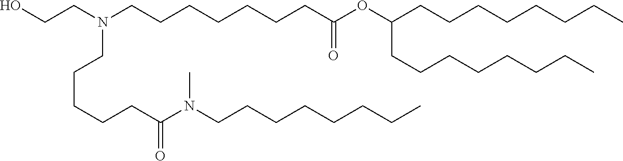 [SMILES: CCCCCCCCC(CCCCCCCC)OC(=O)CCCCCCCN(CCO)CCCCCC(=O)N(C)CCCCCCCC] |
| I 147 | 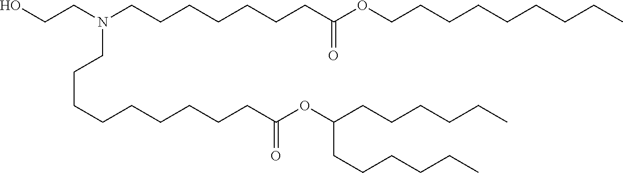 [SMILES: CCCCCCCCCOC(=O)CCCCCCCN(CCO)CCCCCCCCCC(=O)OC(CCCCCC)CCCCCC] |
| I 148 | 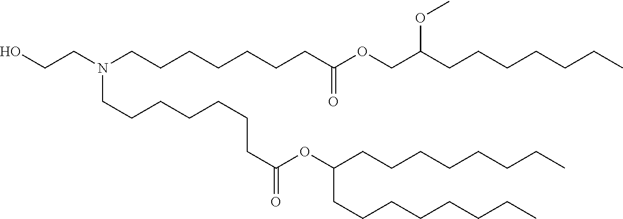 [SMILES: CCCCCCCCC(CCCCCCCC)OC(=O)CCCCCCCN(CCO)CCCCCCCC(=O)OCC(CCCCCCC)OC] |
| I 149 | 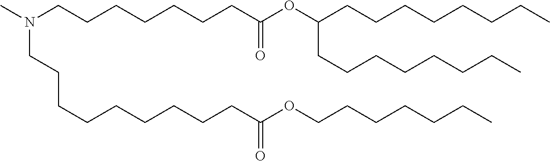 [SMILES: CCCCCCCCC(CCCCCCCC)OC(=O)CCCCCCCN(C)CCCCCCCCCC(=O)OCCCCCCC] |
| I 150 | 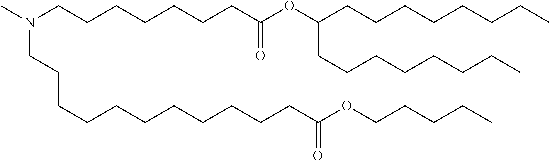 [SMILES: CCCCCCCCC(CCCCCCCC)OC(=O)CCCCCCCN(C)CCCCCCCCCCCC(=O)OCCCCC] |
| I 151 | 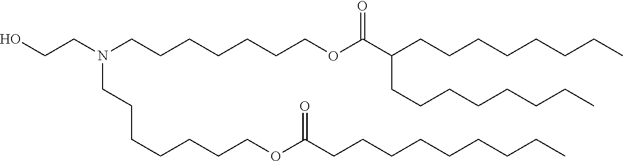 [SMILES: CCCCCCCCCC(=O)OCCCCCCCN(CCO)CCCCCCCOC(=O)C(CCCCCCCC)CCCCCCCC] |
| I 152 | 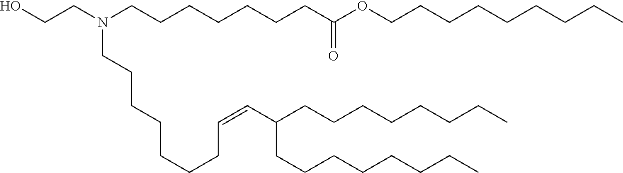 [SMILES: CCCCCCCCCOC(=O)CCCCCCCN(CCO)CCCCCCC/C=C\C(CCCCCCCC)CCCCCCCC] |
| I 153 | 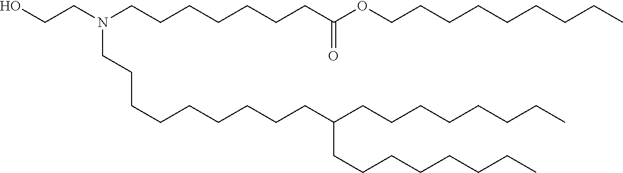 [SMILES: CCCCCCCCCOC(=O)CCCCCCCN(CCO)CCCCCCCCCC(CCCCCCCC)CCCCCCCC] |
| I 154 | 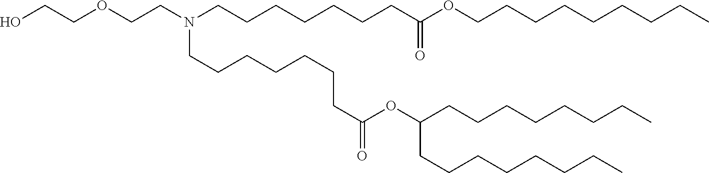 [SMILES: CCCCCCCCCOC(=O)CCCCCCCN(CCCCCCCC(=O)OC(CCCCCCCC)CCCCCCCC)CCOCCO] |
| I 155 | 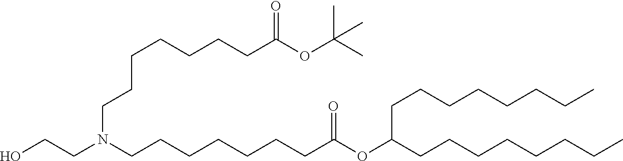 [SMILES: CCCCCCCCC(CCCCCCCC)OC(=O)CCCCCCCN(CCO)CCCCCCCC(=O)OC(C)(C)C] |
| I 156 | 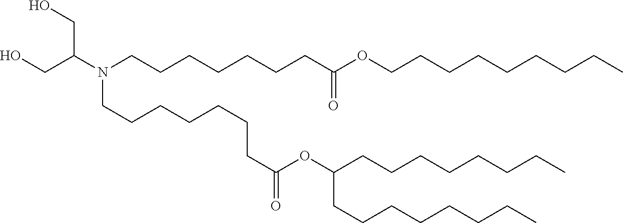 [SMILES: CCCCCCCCCOC(=O)CCCCCCCN(CCCCCCCC(=O)OC(CCCCCCCC)CCCCCCCC)C(CO)CO] |
| I 157 | 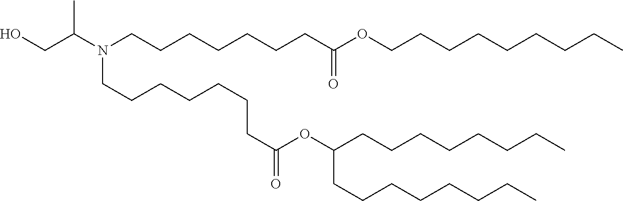 [SMILES: CCCCCCCCCOC(=O)CCCCCCCN(CCCCCCCC(=O)OC(CCCCCCCC)CCCCCCCC)C(C)CO] |
| I 158 | 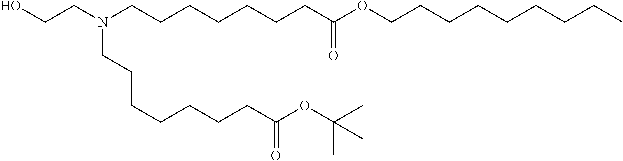 [SMILES: CCCCCCCCCOC(=O)CCCCCCCN(CCO)CCCCCCCC(=O)OC(C)(C)C] |
| I 159 | 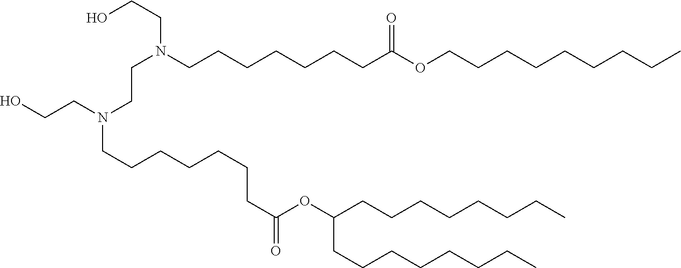 [SMILES: CCCCCCCCCOC(=O)CCCCCCCN(CCO)CCN(CCO)CCCCCCCC(=O)OC(CCCCCCCC)CCCCCCCC] |
| I 160 | 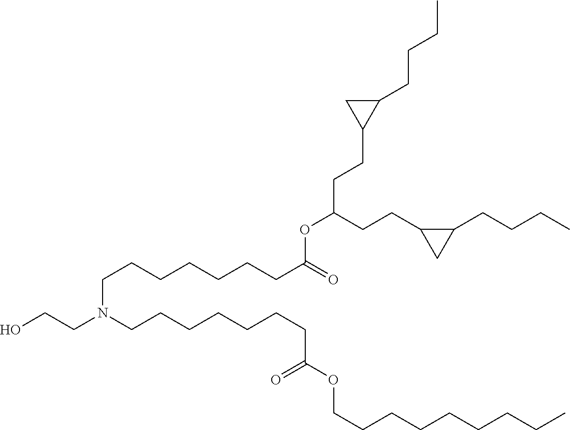 [SMILES: CCCCCCCCCOC(=O)CCCCCCCN(CCO)CCCCCCCC(=O)OC(CCC1CC1CCCC)CCC1CC1CCCC] |
| I 161 | 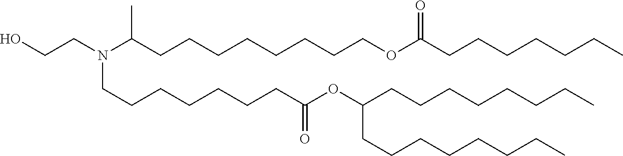 [SMILES: CCCCCCCCC(CCCCCCCC)OC(=O)CCCCCCCN(CCO)C(C)CCCCCCCCOC(=O)CCCCCCC] |
| I 162 | 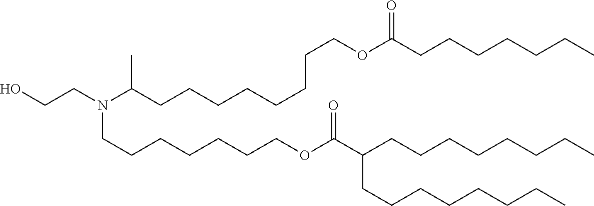 [SMILES: CCCCCCCCC(CCCCCCCC)C(=O)OCCCCCCCN(CCO)C(C)CCCCCCCCOC(=O)CCCCCCC] |
| I 163 | 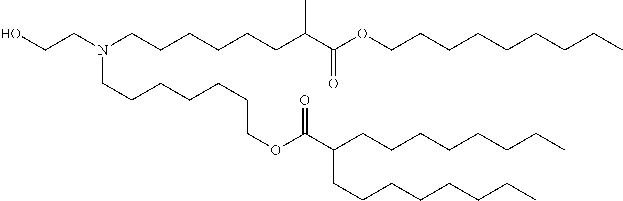 [SMILES: CCCCCCCCCOC(=O)C(C)CCCCCCN(CCO)CCCCCCCOC(=O)C(CCCCCCCC)CCCCCCCC] |
| I 164 | 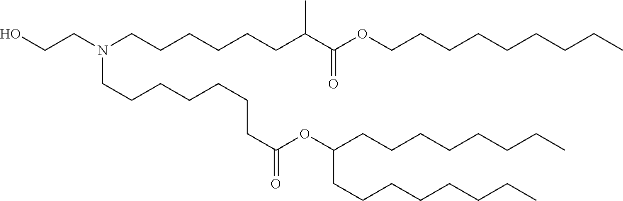 [SMILES: CCCCCCCCCOC(=O)C(C)CCCCCCN(CCO)CCCCCCCC(=O)OC(CCCCCCCC)CCCCCCCC] |
| I 165 | 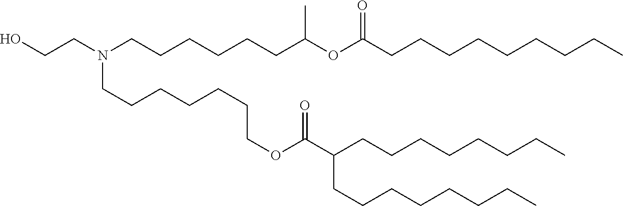 [SMILES: CCCCCCCCCC(=O)OC(C)CCCCCCN(CCO)CCCCCCCOC(=O)C(CCCCCCCC)CCCCCCCC] |
| I 166 | 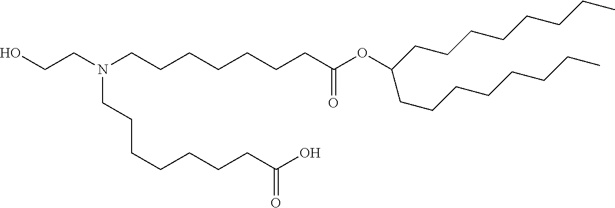 [SMILES: CCCCCCCCC(CCCCCCCC)OC(=O)CCCCCCCN(CCO)CCCCCCCC(=O)O] |
| I 167 | 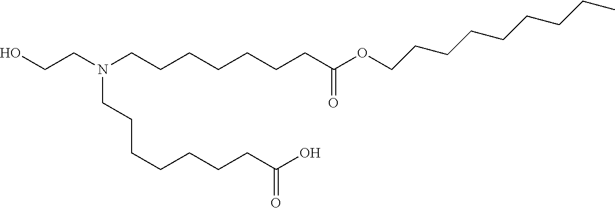 [SMILES: CCCCCCCCCOC(=O)CCCCCCCN(CCO)CCCCCCCC(=O)O] |
| I 168 | 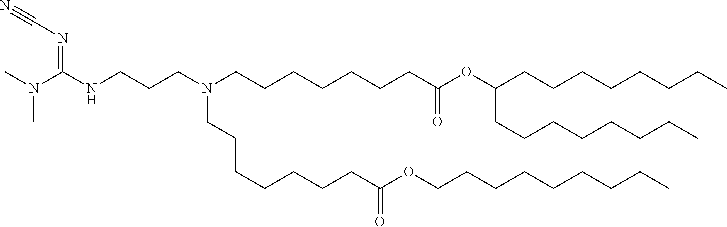 [SMILES: CCCCCCCCCOC(=O)CCCCCCCN(CCCCCCCC(=O)OC(CCCCCCCC)CCCCCCCC)CCCN/C(=N/C#N)N(C)C] |
| I 169 | 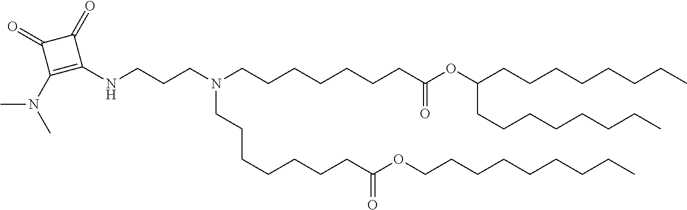 [SMILES: CCCCCCCCCOC(=O)CCCCCCCN(CCCCCCCC(=O)OC(CCCCCCCC)CCCCCCCC)CCCNc1c(N(C)C)c(=O)c1=O] |
| I 170 | 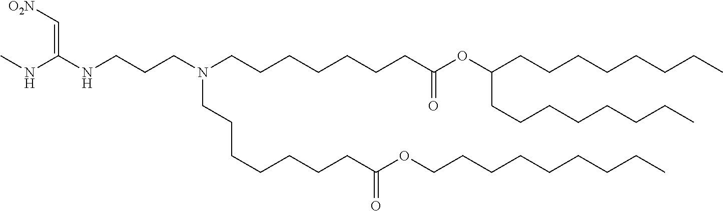 [SMILES: CCCCCCCCCOC(=O)CCCCCCCN(CCCCCCCC(=O)OC(CCCCCCCC)CCCCCCCC)CCCN/C(=C/[N+](=O)[O-])NC] |
| I 171 | 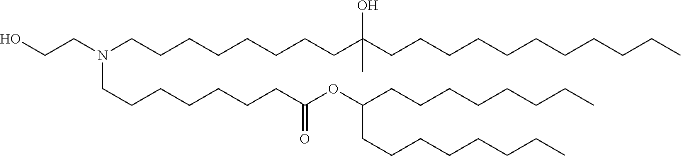 [SMILES: CCCCCCCCCCCC(C)(O)CCCCCCCCN(CCO)CCCCCCCC(=O)OC(CCCCCCCC)CCCCCCCC] |
| I 172 | 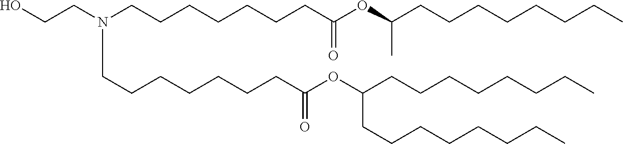 [SMILES: CCCCCCCCC(CCCCCCCC)OC(=O)CCCCCCCN(CCO)CCCCCCCC(=O)O[C@H](C)CCCCCCCC] |
| I 173 | 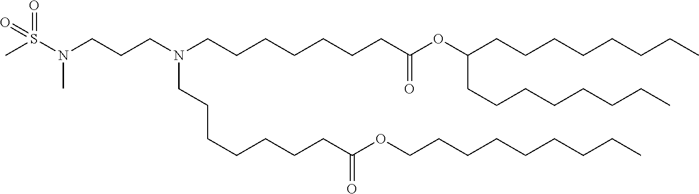 [SMILES: CCCCCCCCCOC(=O)CCCCCCCN(CCCCCCCC(=O)OC(CCCCCCCC)CCCCCCCC)CCCN(C)S(C)(=O)=O] |
| I 174 | 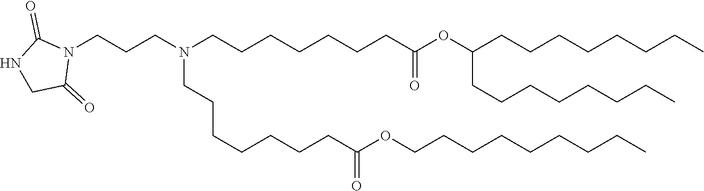 [SMILES: CCCCCCCCCOC(=O)CCCCCCCN(CCCCCCCC(=O)OC(CCCCCCCC)CCCCCCCC)CCCN1C(=O)CNC1=O] |
| I 175 | 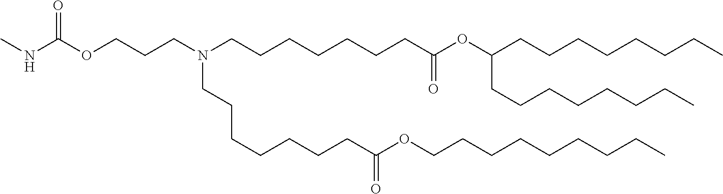 [SMILES: CCCCCCCCCOC(=O)CCCCCCCN(CCCCCCCC(=O)OC(CCCCCCCC)CCCCCCCC)CCCOC(=O)NC] |
| I 176 | 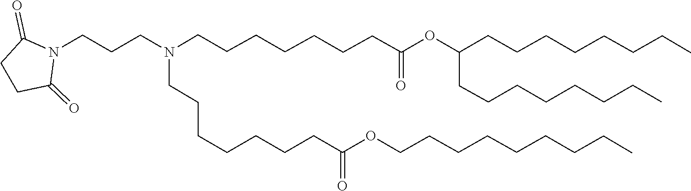 [SMILES: CCCCCCCCCOC(=O)CCCCCCCN(CCCCCCCC(=O)OC(CCCCCCCC)CCCCCCCC)CCCN1C(=O)CCC1=O] |
| I 177 | 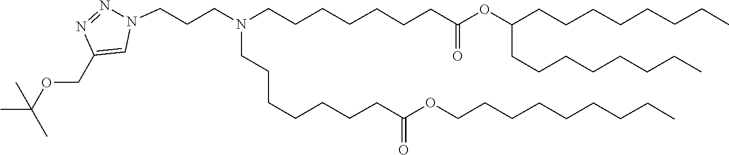 [SMILES: CCCCCCCCCOC(=O)CCCCCCCN(CCCCCCCC(=O)OC(CCCCCCCC)CCCCCCCC)CCCn1cc(COC(C)(C)C)nn1] |
| I 178 | 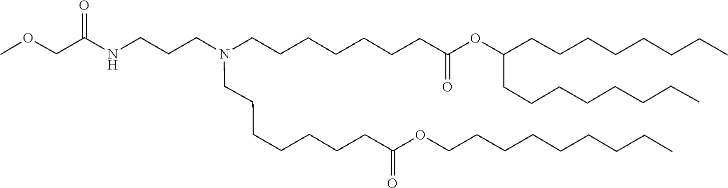 [SMILES: CCCCCCCCCOC(=O)CCCCCCCN(CCCCCCCC(=O)OC(CCCCCCCC)CCCCCCCC)CCCNC(=O)COC] |
| I 179 | 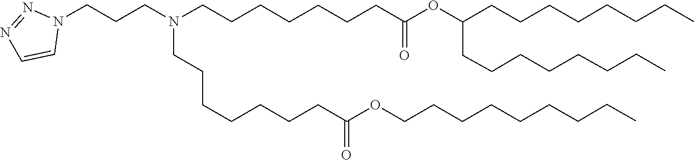 [SMILES: CCCCCCCCCOC(=O)CCCCCCCN(CCCCCCCC(=O)OC(CCCCCCCC)CCCCCCCC)CCCn1ccnn1] |
| I 180 | 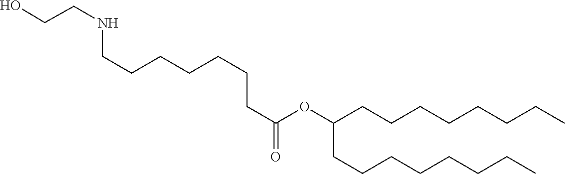 [SMILES: CCCCCCCCC(CCCCCCCC)OC(=O)CCCCCCCNCCO] |
| I 181 | 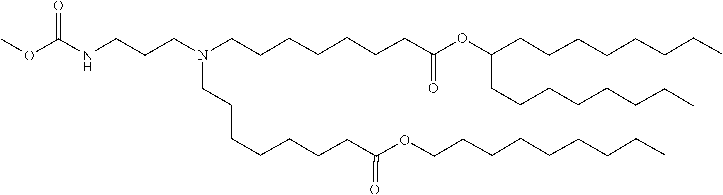 [SMILES: CCCCCCCCCOC(=O)CCCCCCCN(CCCCCCCC(=O)OC(CCCCCCCC)CCCCCCCC)CCCNC(=O)OC] |
| I 182 | 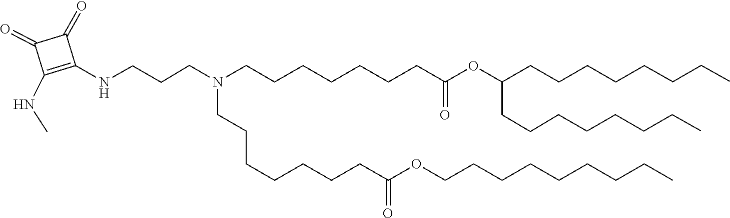 [SMILES: CCCCCCCCCOC(=O)CCCCCCCN(CCCCCCCC(=O)OC(CCCCCCCC)CCCCCCCC)CCCNc1c(NC)c(=O)c1=O] |
| I 183 | 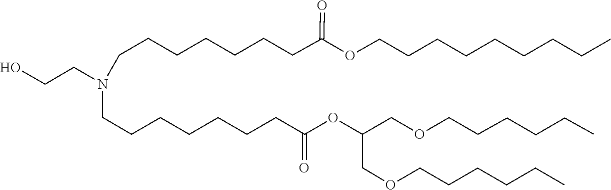 [SMILES: CCCCCCCCCOC(=O)CCCCCCCN(CCO)CCCCCCCC(=O)OC(COCCCCCC)COCCCCCC] |
| I 184 | 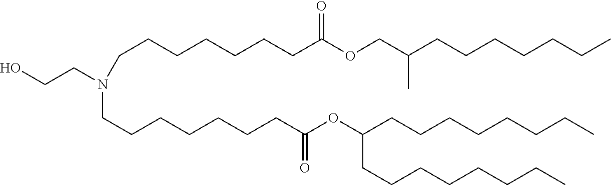 [SMILES: CCCCCCCCC(CCCCCCCC)OC(=O)CCCCCCCN(CCO)CCCCCCCC(=O)OCC(C)CCCCCCC] |
| I 185 | 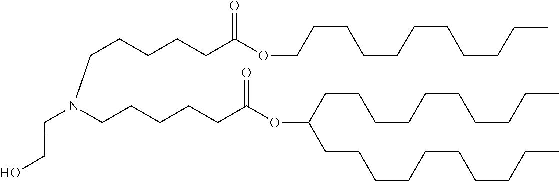 [SMILES: CCCCCCCCCCCOC(=O)CCCCCN(CCO)CCCCCC(=O)OC(CCCCCCCCCC)CCCCCCCCCC] |
| I 186 | 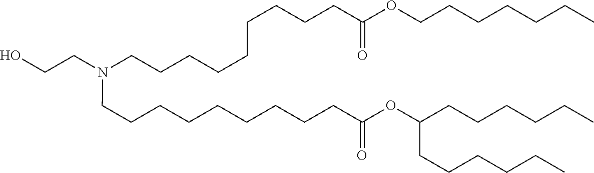 [SMILES: CCCCCCCOC(=O)CCCCCCCCCN(CCO)CCCCCCCCCC(=O)OC(CCCCCC)CCCCCC] |
| I 187 | 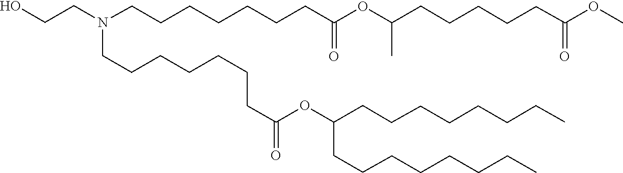 [SMILES: CCCCCCCCC(CCCCCCCC)OC(=O)CCCCCCCN(CCO)CCCCCCCC(=O)OC(C)CCCCCC(=O)OC] |
| I 188 | 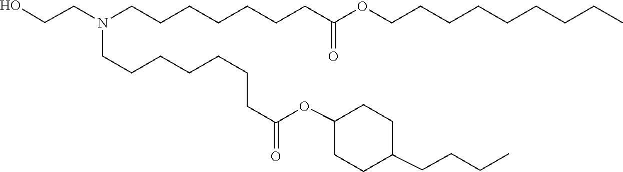 [SMILES: CCCCCCCCCOC(=O)CCCCCCCN(CCO)CCCCCCCC(=O)OC1CCC(CCCC)CC1] |
| I 189 | 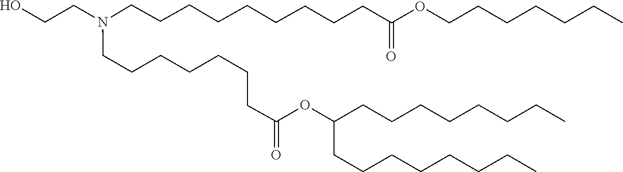 [SMILES: CCCCCCCCC(CCCCCCCC)OC(=O)CCCCCCCN(CCO)CCCCCCCCCC(=O)OCCCCCCC] |
| I 190 | 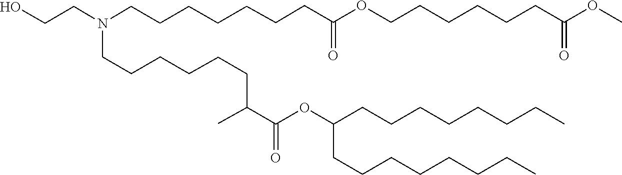 [SMILES: CCCCCCCCC(CCCCCCCC)OC(=O)C(C)CCCCCCN(CCO)CCCCCCCC(=O)OCCCCCCC(=O)OC] |
| I 191 | 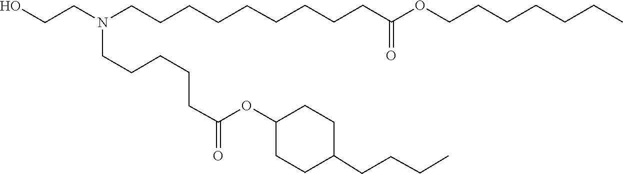 [SMILES: CCCCCCCOC(=O)CCCCCCCCCN(CCO)CCCCCC(=O)OC1CCC(CCCC)CC1] |
| I 192 | 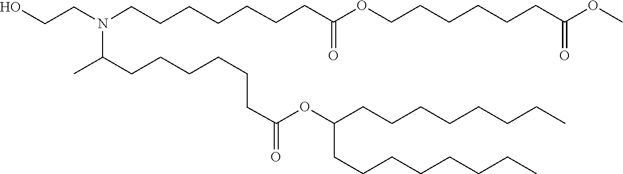 [SMILES: CCCCCCCCC(CCCCCCCC)OC(=O)CCCCCCC(C)N(CCO)CCCCCCCC(=O)OCCCCCCC(=O)OC] |
| I 193 | 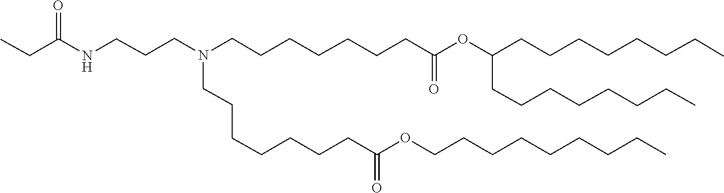 [SMILES: CCCCCCCCCOC(=O)CCCCCCCN(CCCCCCCC(=O)OC(CCCCCCCC)CCCCCCCC)CCCNC(=O)CC] |
| I 194 | 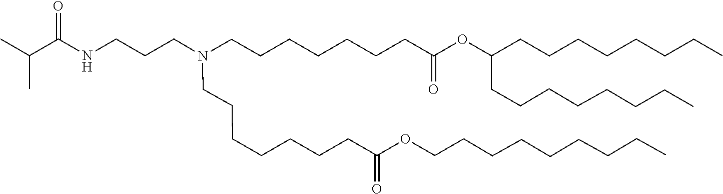 [SMILES: CCCCCCCCCOC(=O)CCCCCCCN(CCCCCCCC(=O)OC(CCCCCCCC)CCCCCCCC)CCCNC(=O)C(C)C] |
| I 195 | 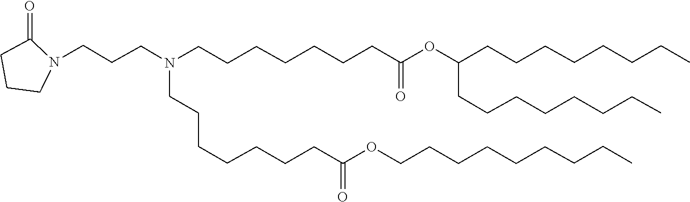 [SMILES: CCCCCCCCCOC(=O)CCCCCCCN(CCCCCCCC(=O)OC(CCCCCCCC)CCCCCCCC)CCCN1CCCC1=O] |
| I 196 | 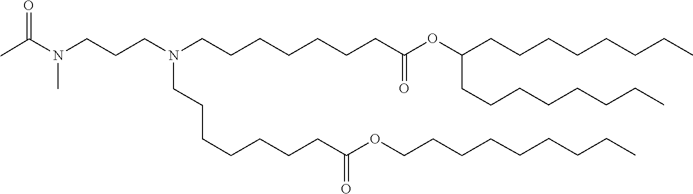 [SMILES: CCCCCCCCCOC(=O)CCCCCCCN(CCCCCCCC(=O)OC(CCCCCCCC)CCCCCCCC)CCCN(C)C(C)=O] |
| I 197 | 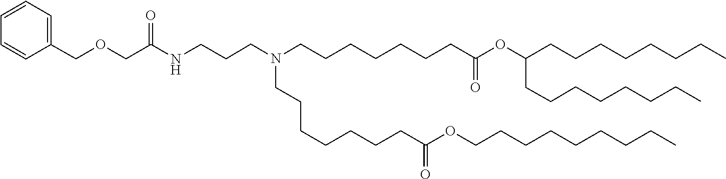 [SMILES: CCCCCCCCCOC(=O)CCCCCCCN(CCCCCCCC(=O)OC(CCCCCCCC)CCCCCCCC)CCCNC(=O)COCc1ccccc1] |
| I 198 | 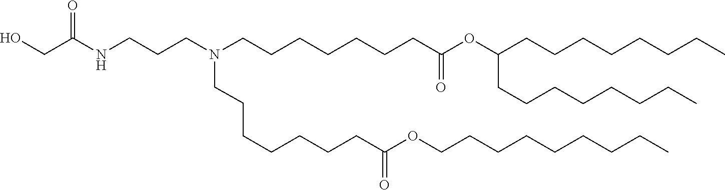 [SMILES: CCCCCCCCCOC(=O)CCCCCCCN(CCCCCCCC(=O)OC(CCCCCCCC)CCCCCCCC)CCCNC(=O)CO] |
| I 199 | 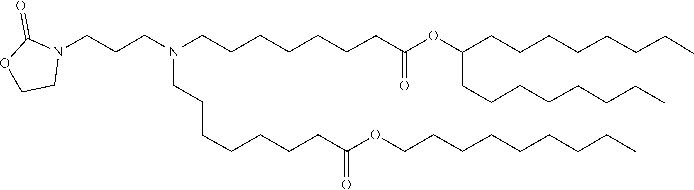 [SMILES: CCCCCCCCCOC(=O)CCCCCCCN(CCCCCCCC(=O)OC(CCCCCCCC)CCCCCCCC)CCCN1CCOC1=O] |
| I 200 | 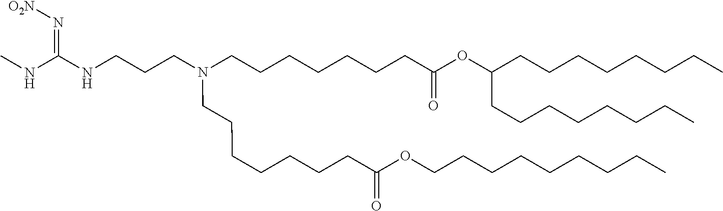 [SMILES: CCCCCCCCCOC(=O)CCCCCCCN(CCCCCCCC(=O)OC(CCCCCCCC)CCCCCCCC)CCCN/C(=N/[N+](=O)[O-])NC] |
| I 201 | 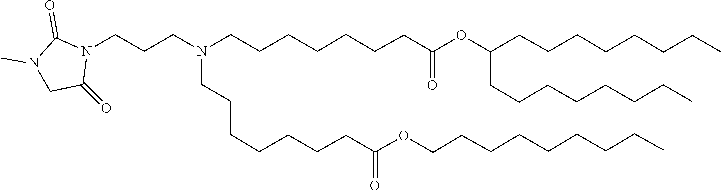 [SMILES: CCCCCCCCCOC(=O)CCCCCCCN(CCCCCCCC(=O)OC(CCCCCCCC)CCCCCCCC)CCCN1C(=O)CN(C)C1=O] |
| I 202 | 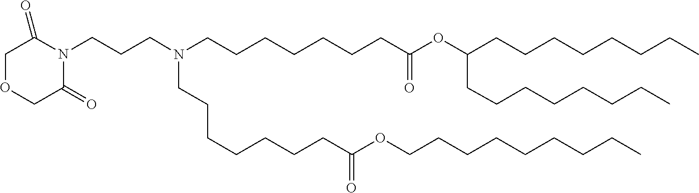 [SMILES: CCCCCCCCCOC(=O)CCCCCCCN(CCCCCCCC(=O)OC(CCCCCCCC)CCCCCCCC)CCCN1C(=O)COCC1=O] |
| I 203 | 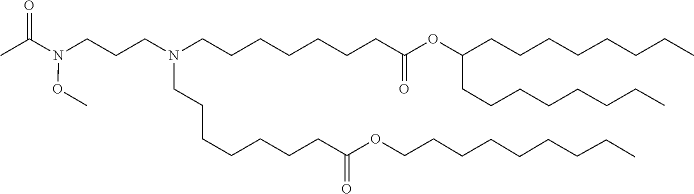 [SMILES: CCCCCCCCCOC(=O)CCCCCCCN(CCCCCCCC(=O)OC(CCCCCCCC)CCCCCCCC)CCCN(OC)C(C)=O] |
| I 204 | 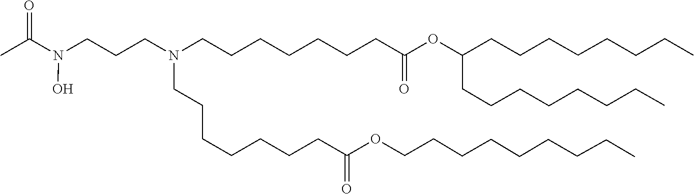 [SMILES: CCCCCCCCCOC(=O)CCCCCCCN(CCCCCCCC(=O)OC(CCCCCCCC)CCCCCCCC)CCCN(O)C(C)=O] |
| I 205 | 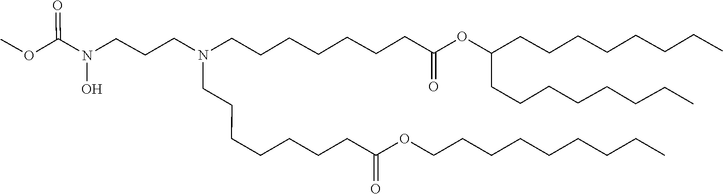 [SMILES: CCCCCCCCCOC(=O)CCCCCCCN(CCCCCCCC(=O)OC(CCCCCCCC)CCCCCCCC)CCCN(O)C(=O)OC] |
| I 206 | 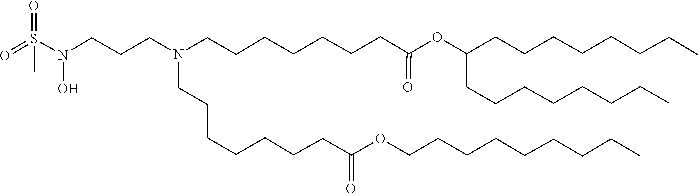 [SMILES: CCCCCCCCCOC(=O)CCCCCCCN(CCCCCCCC(=O)OC(CCCCCCCC)CCCCCCCC)CCCN(O)S(C)(=O)=O] |
| I 207 | 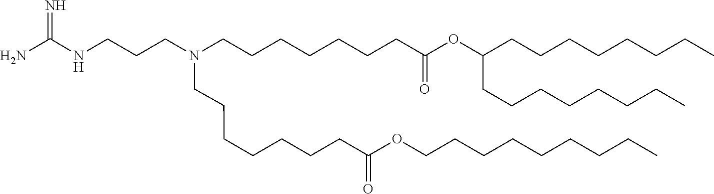 [SMILES: CCCCCCCCCOC(=O)CCCCCCCN(CCCCCCCC(=O)OC(CCCCCCCC)CCCCCCCC)CCCNC(=N)N] |
| I 208 | 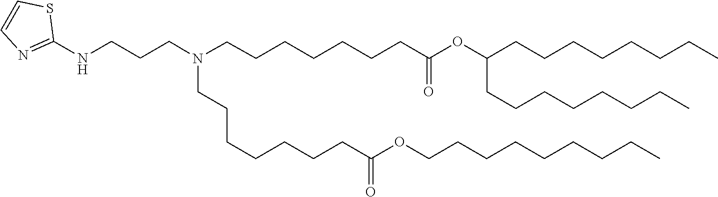 [SMILES: CCCCCCCCCOC(=O)CCCCCCCN(CCCCCCCC(=O)OC(CCCCCCCC)CCCCCCCC)CCCNc1nccs1] |
| I 209 | 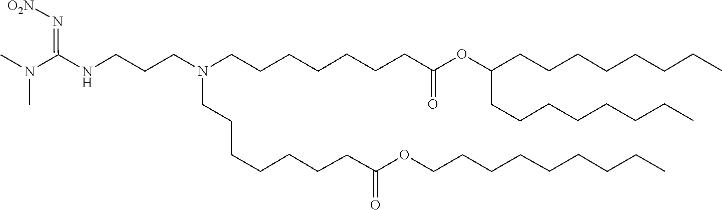 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I 210 | 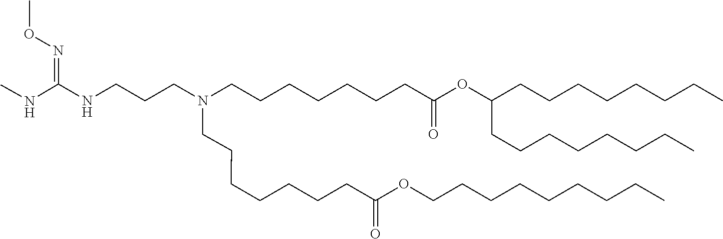 [SMILES: CCCCCCCCCOC(=O)CCCCCCCN(CCCCCCCC(=O)OC(CCCCCCCC)CCCCCCCC)CCCN/C(=N/OC)NC] |
| I 211 | 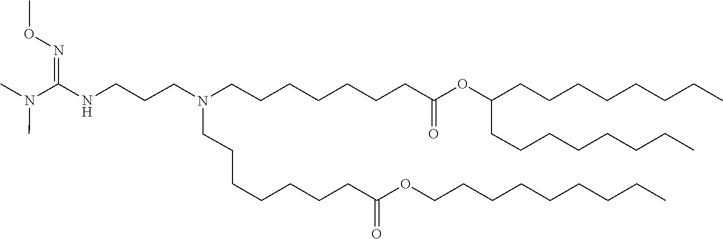 [SMILES: CCCCCCCCCOC(=O)CCCCCCCN(CCCCCCCC(=O)OC(CCCCCCCC)CCCCCCCC)CCCN/C(=N/OC)N(C)C] |
| I 212 | 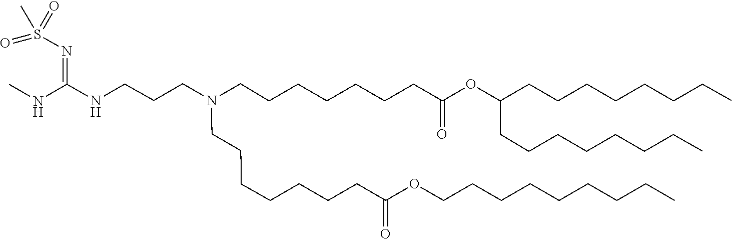 [SMILES: CCCCCCCCCOC(=O)CCCCCCCN(CCCCCCCC(=O)OC(CCCCCCCC)CCCCCCCC)CCCN/C(=N/S(C)(=O)=O)NC] |
| I 213 | 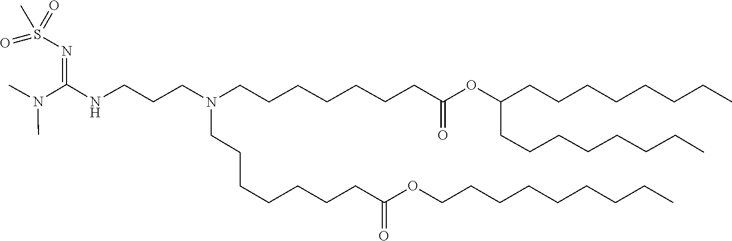 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I 214 | 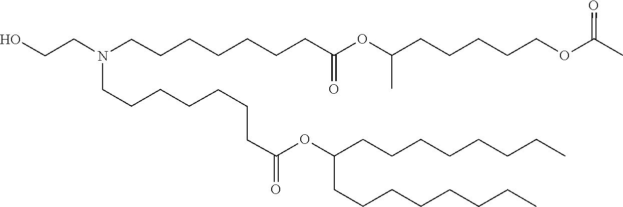 [SMILES: CCCCCCCCC(CCCCCCCC)OC(=O)CCCCCCCN(CCO)CCCCCCCC(=O)OC(C)CCCCCOC(C)=O] |
| I 215 | 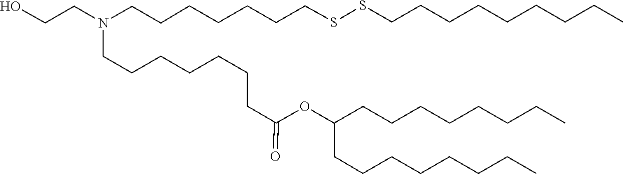 [SMILES: CCCCCCCCCSSCCCCCCCN(CCO)CCCCCCCC(=O)OC(CCCCCCCC)CCCCCCCC] |
| I 216 | 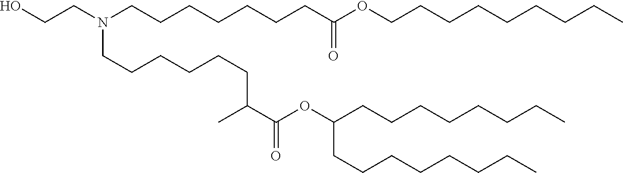 [SMILES: CCCCCCCCCOC(=O)CCCCCCCN(CCO)CCCCCCC(C)C(=O)OC(CCCCCCCC)CCCCCCCC] |
| I 217 | 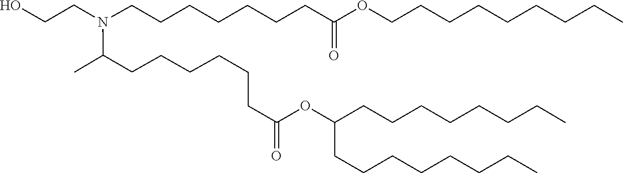 [SMILES: CCCCCCCCCOC(=O)CCCCCCCN(CCO)C(C)CCCCCCC(=O)OC(CCCCCCCC)CCCCCCCC] |
| I 218 | 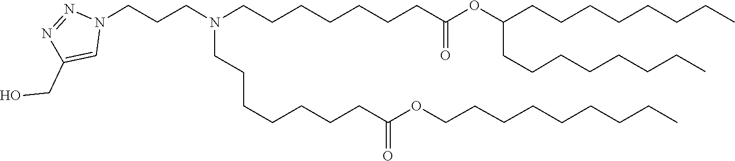 [SMILES: CCCCCCCCCOC(=O)CCCCCCCN(CCCCCCCC(=O)OC(CCCCCCCC)CCCCCCCC)CCCn1cc(CO)nn1] |
| I 219 | 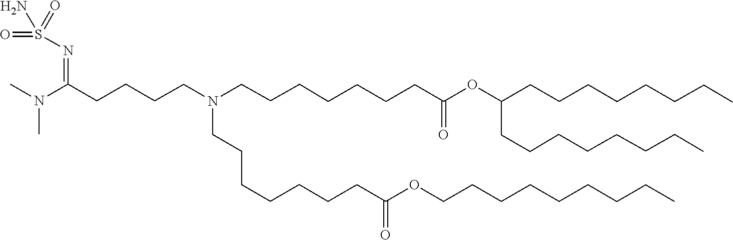 [SMILES: CCCCCCCCCOC(=O)CCCCCCCN(CCCCCCCC(=O)OC(CCCCCCCC)CCCCCCCC)CCCC/C(=N/S(N)(=O)=O)N(C)C] |
| I 220 | 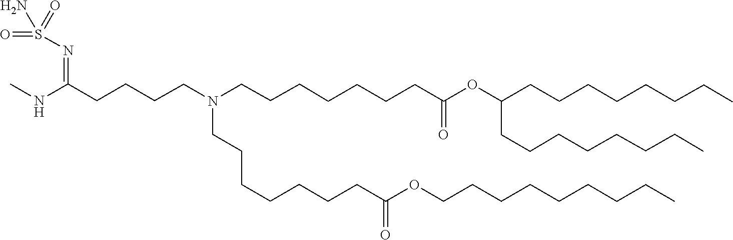 [SMILES: CCCCCCCCCOC(=O)CCCCCCCN(CCCCCCCC(=O)OC(CCCCCCCC)CCCCCCCC)CCCC/C(=N/S(N)(=O)=O)NC] |
| I 221 | 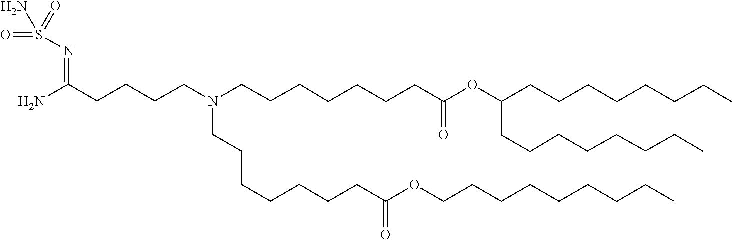 [SMILES: CCCCCCCCCOC(=O)CCCCCCCN(CCCCCCCC(=O)OC(CCCCCCCC)CCCCCCCC)CCCC/C(N)=N/S(N)(=O)=O] |
| I 222 | 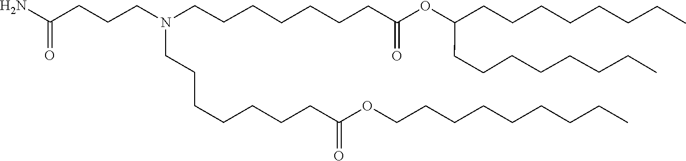 [SMILES: CCCCCCCCCOC(=O)CCCCCCCN(CCCCCCCC(=O)OC(CCCCCCCC)CCCCCCCC)CCCC(N)=O] |
| I 223 | 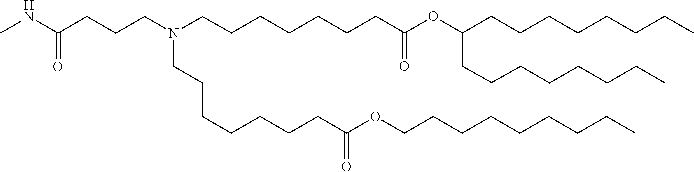 [SMILES: CCCCCCCCCOC(=O)CCCCCCCN(CCCCCCCC(=O)OC(CCCCCCCC)CCCCCCCC)CCCC(=O)NC] |
| I 224 | 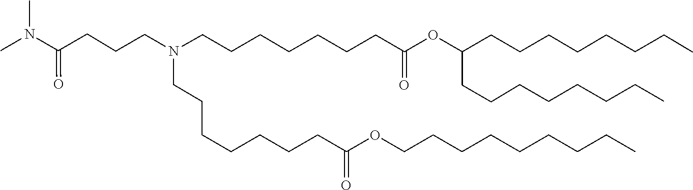 [SMILES: CCCCCCCCCOC(=O)CCCCCCCN(CCCCCCCC(=O)OC(CCCCCCCC)CCCCCCCC)CCCC(=O)N(C)C] |
| I 225 | 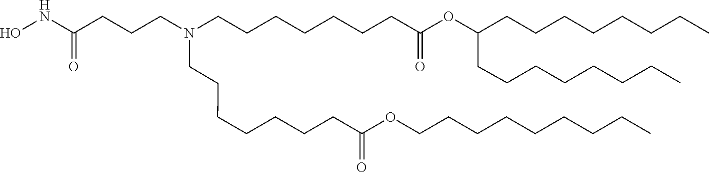 [SMILES: CCCCCCCCCOC(=O)CCCCCCCN(CCCCCCCC(=O)OC(CCCCCCCC)CCCCCCCC)CCCC(=O)NO] |
| I 226 | 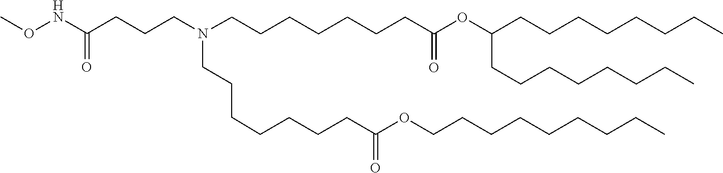 [SMILES: CCCCCCCCCOC(=O)CCCCCCCN(CCCCCCCC(=O)OC(CCCCCCCC)CCCCCCCC)CCCC(=O)NOC] |
| I 227 | 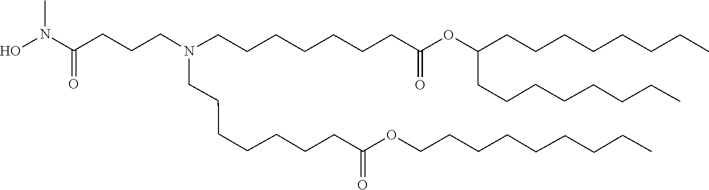 [SMILES: CCCCCCCCCOC(=O)CCCCCCCN(CCCCCCCC(=O)OC(CCCCCCCC)CCCCCCCC)CCCC(=O)N(C)O] |
| I 228 | 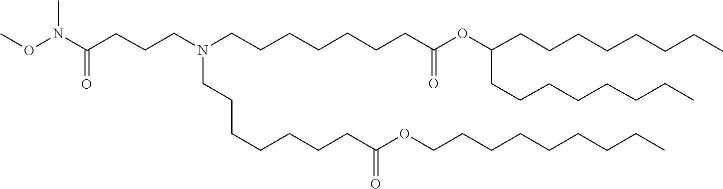 [SMILES: CCCCCCCCCOC(=O)CCCCCCCN(CCCCCCCC(=O)OC(CCCCCCCC)CCCCCCCC)CCCC(=O)N(C)OC] |
| I 229 | 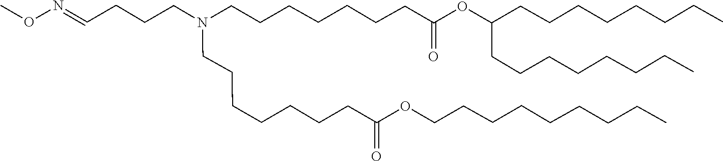 [SMILES: CCCCCCCCCOC(=O)CCCCCCCN(CCC/C=N/OC)CCCCCCCC(=O)OC(CCCCCCCC)CCCCCCCC] |
| I 230 | 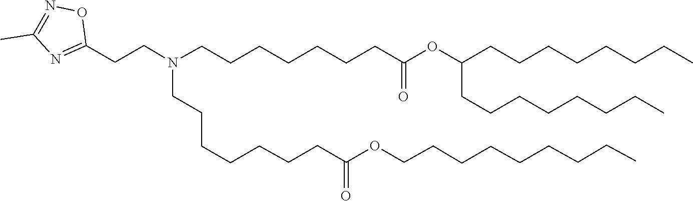 [SMILES: CCCCCCCCCOC(=O)CCCCCCCN(CCCCCCCC(=O)OC(CCCCCCCC)CCCCCCCC)CCc1nc(C)no1] |
| I 231 | 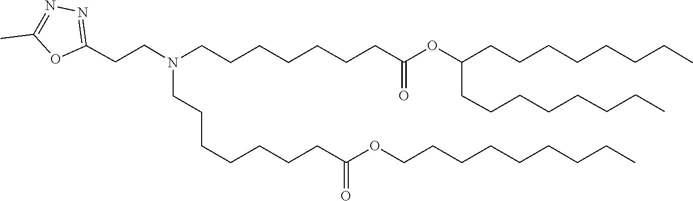 [SMILES: CCCCCCCCCOC(=O)CCCCCCCN(CCCCCCCC(=O)OC(CCCCCCCC)CCCCCCCC)CCc1nnc(C)o1] |
| I 232 | 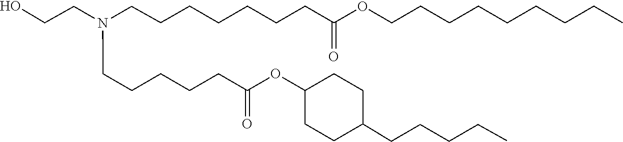 [SMILES: CCCCCCCCCOC(=O)CCCCCCCN(CCO)CCCCCC(=O)OC1CCC(CCCCC)CC1] |
| I 233 | 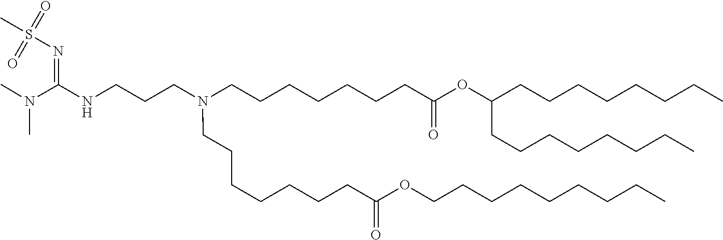 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I 234 | 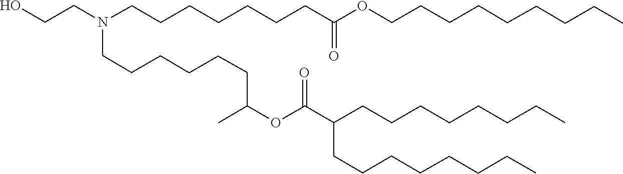 [SMILES: CCCCCCCCCOC(=O)CCCCCCCN(CCO)CCCCCCC(C)OC(=O)C(CCCCCCCC)CCCCCCCC] |
| I 235 | 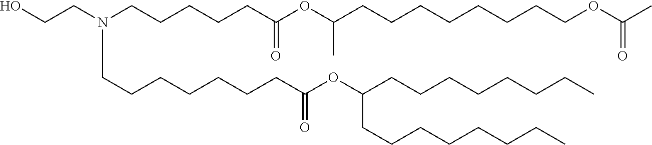 [SMILES: CCCCCCCCC(CCCCCCCC)OC(=O)CCCCCCCN(CCO)CCCCCC(=O)OC(C)CCCCCCCCOC(C)=O] |
| I 236 | 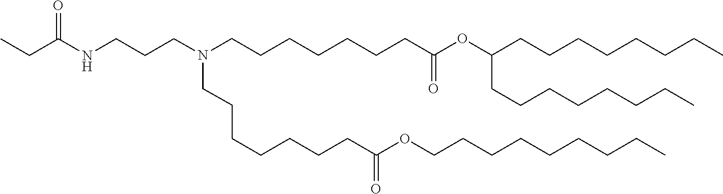 [SMILES: CCCCCCCCCOC(=O)CCCCCCCN(CCCCCCCC(=O)OC(CCCCCCCC)CCCCCCCC)CCCNC(=O)CC] |
| I 237 | 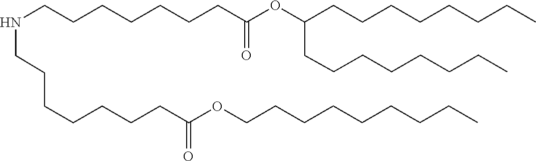 [SMILES: CCCCCCCCCOC(=O)CCCCCCCNCCCCCCCC(=O)OC(CCCCCCCC)CCCCCCCC] |
| I 238 | 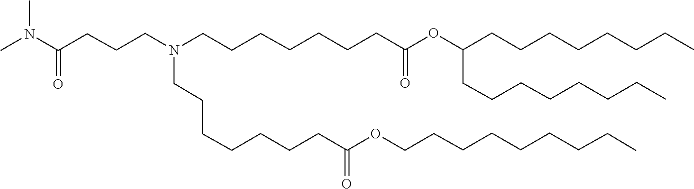 [SMILES: CCCCCCCCCOC(=O)CCCCCCCN(CCCCCCCC(=O)OC(CCCCCCCC)CCCCCCCC)CCCC(=O)N(C)C] |
| I 239 | 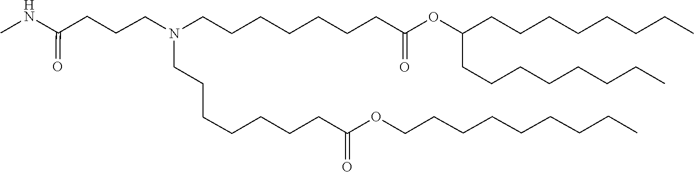 [SMILES: CCCCCCCCCOC(=O)CCCCCCCN(CCCCCCCC(=O)OC(CCCCCCCC)CCCCCCCC)CCCC(=O)NC] |
| I 240 | 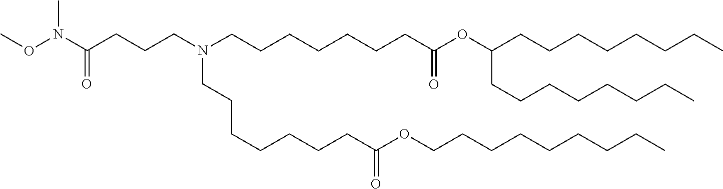 [SMILES: CCCCCCCCCOC(=O)CCCCCCCN(CCCCCCCC(=O)OC(CCCCCCCC)CCCCCCCC)CCCC(=O)N(C)OC] |
| I 241 | 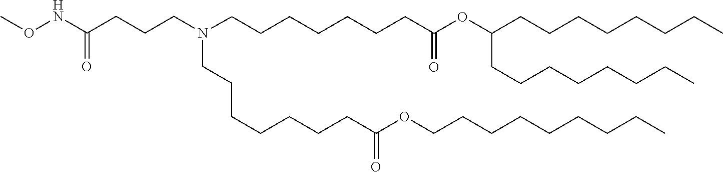 [SMILES: CCCCCCCCCOC(=O)CCCCCCCN(CCCCCCCC(=O)OC(CCCCCCCC)CCCCCCCC)CCCC(=O)NOC] |
| I 242 | 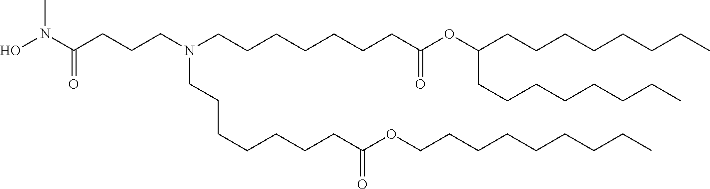 [SMILES: CCCCCCCCCOC(=O)CCCCCCCN(CCCCCCCC(=O)OC(CCCCCCCC)CCCCCCCC)CCCC(=O)N(C)O] |
| I 243 | 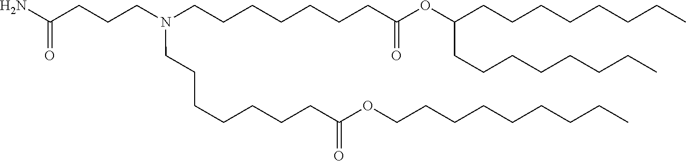 [SMILES: CCCCCCCCCOC(=O)CCCCCCCN(CCCCCCCC(=O)OC(CCCCCCCC)CCCCCCCC)CCCC(N)=O] |
| I 244 | 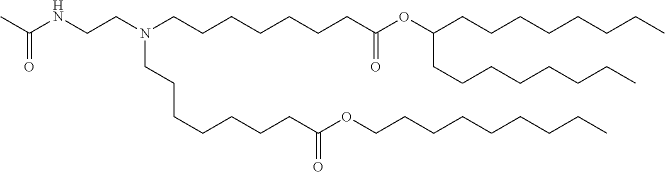 [SMILES: CCCCCCCCCOC(=O)CCCCCCCN(CCCCCCCC(=O)OC(CCCCCCCC)CCCCCCCC)CCNC(C)=O] |
| I 245 | 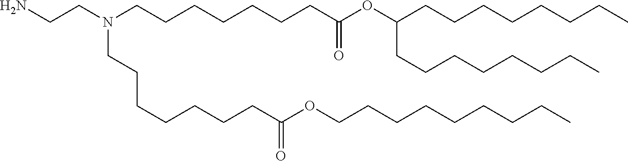 [SMILES: CCCCCCCCCOC(=O)CCCCCCCN(CCN)CCCCCCCC(=O)OC(CCCCCCCC)CCCCCCCC] |
| I 246 | 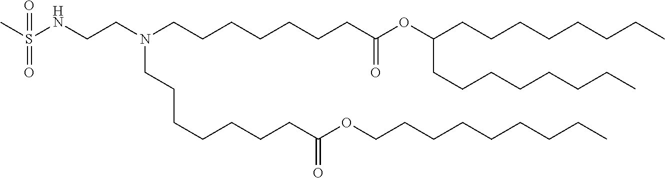 [SMILES: CCCCCCCCCOC(=O)CCCCCCCN(CCCCCCCC(=O)OC(CCCCCCCC)CCCCCCCC)CCNS(C)(=O)=O] |
| I 247 | 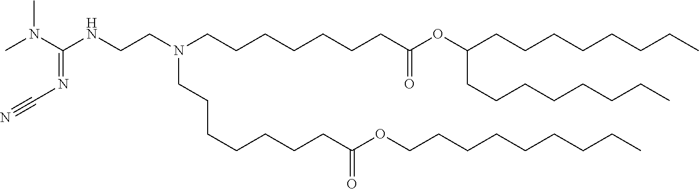 [SMILES: CCCCCCCCCOC(=O)CCCCCCCN(CCCCCCCC(=O)OC(CCCCCCCC)CCCCCCCC)CCN/C(=N/C#N)N(C)C] |
| I 248 | 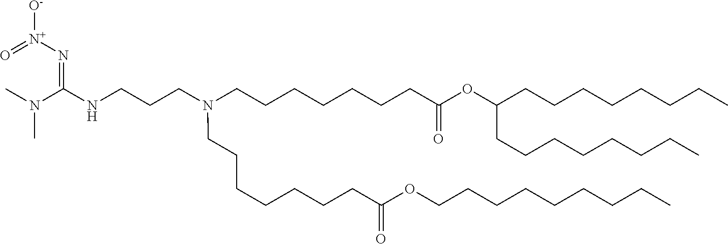 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I 249 | 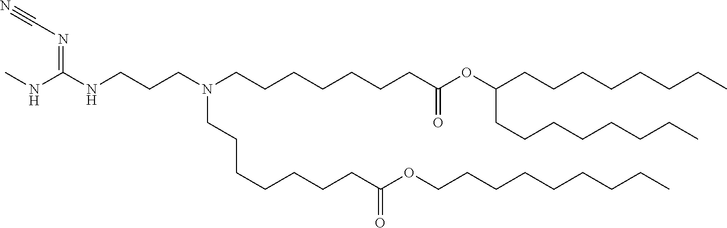 [SMILES: CCCCCCCCCOC(=O)CCCCCCCN(CCCCCCCC(=O)OC(CCCCCCCC)CCCCCCCC)CCCN/C(=N/C#N)NC] |
| I 250 | 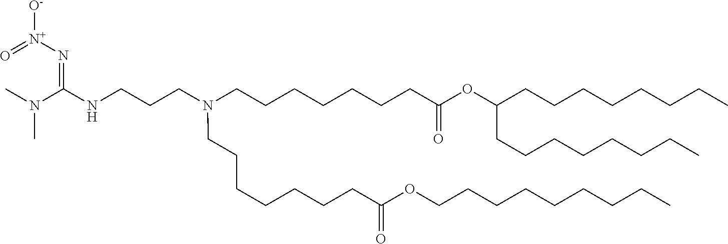 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I 251 | 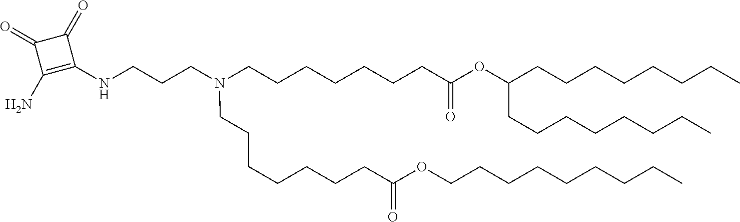 [SMILES: CCCCCCCCCOC(=O)CCCCCCCN(CCCCCCCC(=O)OC(CCCCCCCC)CCCCCCCC)CCCNc1c(N)c(=O)c1=O] |
| I 252 | 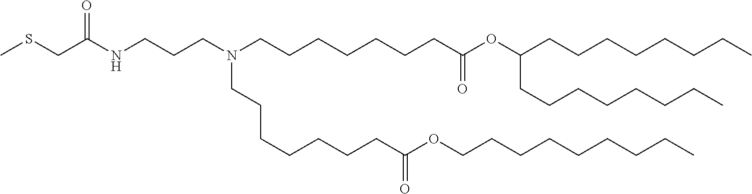 [SMILES: CCCCCCCCCOC(=O)CCCCCCCN(CCCCCCCC(=O)OC(CCCCCCCC)CCCCCCCC)CCCNC(=O)CSC] |
| I 253 | 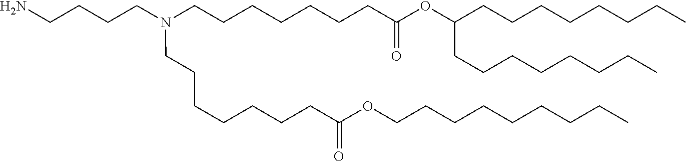 [SMILES: CCCCCCCCCOC(=O)CCCCCCCN(CCCCN)CCCCCCCC(=O)OC(CCCCCCCC)CCCCCCCC] |
| I 254 | 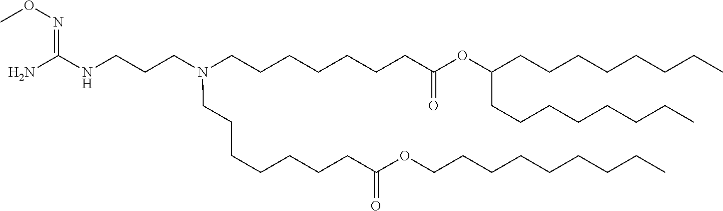 [SMILES: CCCCCCCCCOC(=O)CCCCCCCN(CCCCCCCC(=O)OC(CCCCCCCC)CCCCCCCC)CCCN/C(N)=N/OC] |
| I 255 | 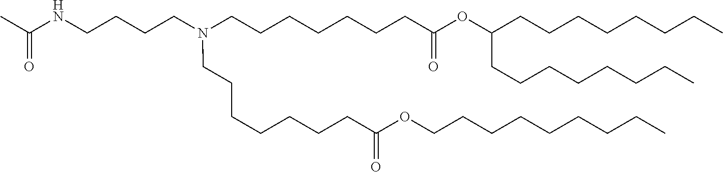 [SMILES: CCCCCCCCCOC(=O)CCCCCCCN(CCCCCCCC(=O)OC(CCCCCCCC)CCCCCCCC)CCCCNC(C)=O] |
| I 256 | 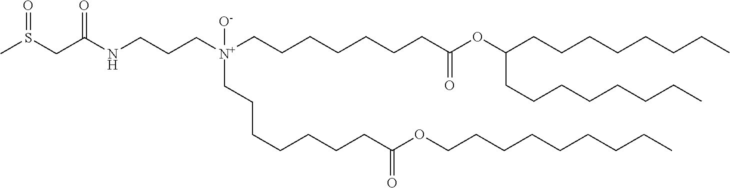 [SMILES: CCCCCCCCCOC(=O)CCCCCCC[N+]([O-])(CCCCCCCC(=O)OC(CCCCCCCC)CCCCCCCC)CCCNC(=O)CS(C)=O] |
| I 257 | 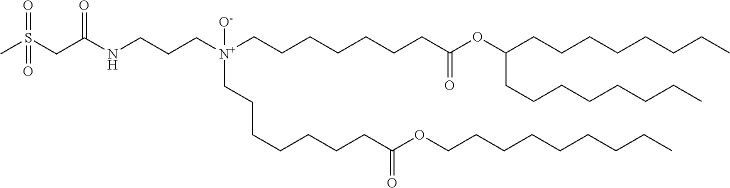 [SMILES: CCCCCCCCCOC(=O)CCCCCCC[N+]([O-])(CCCCCCCC(=O)OC(CCCCCCCC)CCCCCCCC)CCCNC(=O)CS(C)(=O)=O] |
| I 258 | 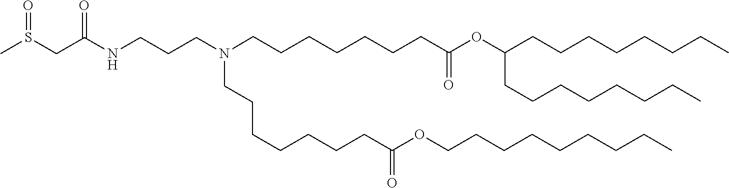 [SMILES: CCCCCCCCCOC(=O)CCCCCCCN(CCCCCCCC(=O)OC(CCCCCCCC)CCCCCCCC)CCCNC(=O)CS(C)=O] |
| I 259 | 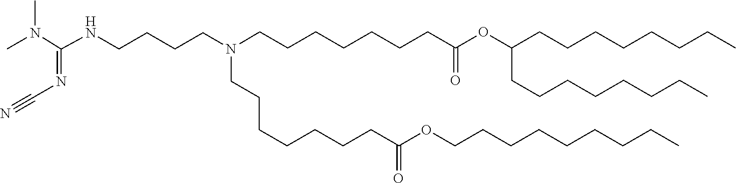 [SMILES: CCCCCCCCCOC(=O)CCCCCCCN(CCCCCCCC(=O)OC(CCCCCCCC)CCCCCCCC)CCCCN/C(=N/C#N)N(C)C] |
| I 260 | 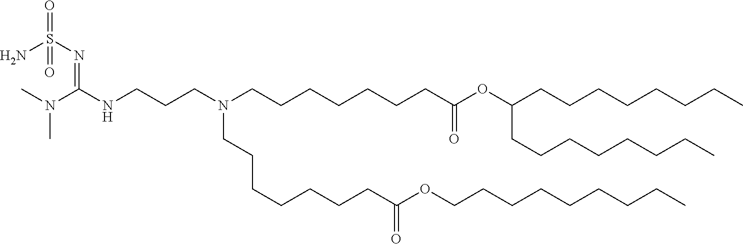 [SMILES: CCCCCCCCCOC(=O)CCCCCCCN(CCCCCCCC(=O)OC(CCCCCCCC)CCCCCCCC)CCCN/C(=N/S(N)(=O)=O)N(C)C] |
| I 261 | 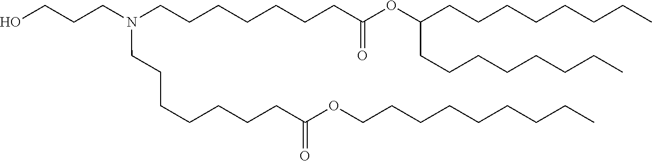 [SMILES: CCCCCCCCCOC(=O)CCCCCCCN(CCCO)CCCCCCCC(=O)OC(CCCCCCCC)CCCCCCCC] |
| I 262 | 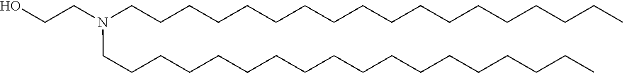 [SMILES: CCCCCCCCCCCCCCCCCCN(CCO)CCCCCCCCCCCCCCCCCC] |
| I 263 | 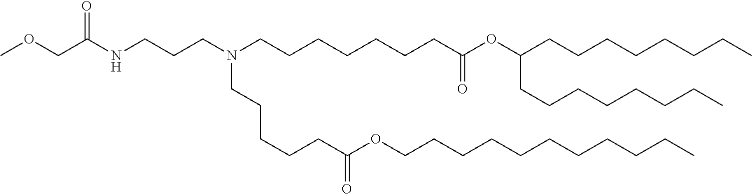 [SMILES: CCCCCCCCCCCOC(=O)CCCCCN(CCCCCCCC(=O)OC(CCCCCCCC)CCCCCCCC)CCCNC(=O)COC] |
| I 264 | 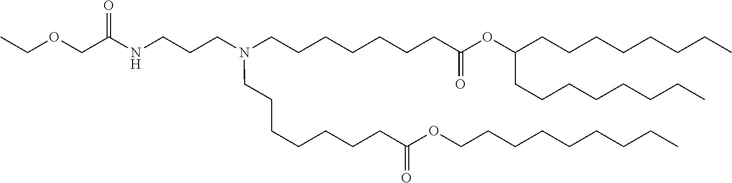 [SMILES: CCCCCCCCCOC(=O)CCCCCCCN(CCCCCCCC(=O)OC(CCCCCCCC)CCCCCCCC)CCCNC(=O)COCC] |
| I 265 | 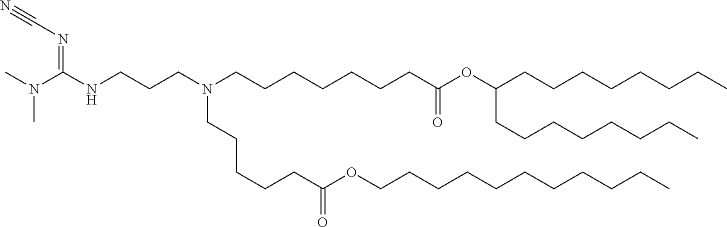 [SMILES: CCCCCCCCCCCOC(=O)CCCCCN(CCCCCCCC(=O)OC(CCCCCCCC)CCCCCCCC)CCCN/C(=N/C#N)N(C)C] |
| I 266 | 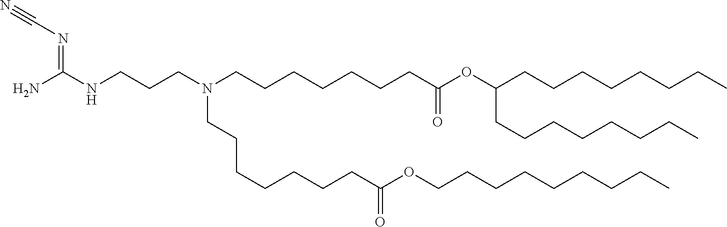 [SMILES: CCCCCCCCCOC(=O)CCCCCCCN(CCCCCCCC(=O)OC(CCCCCCCC)CCCCCCCC)CCCN/C(N)=N/C#N] |
| I 267 | 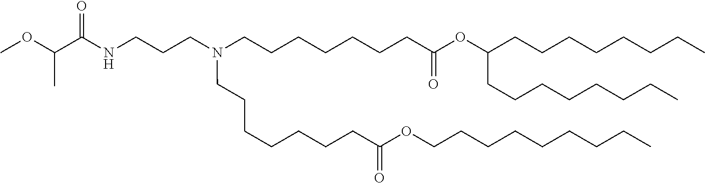 [SMILES: CCCCCCCCCOC(=O)CCCCCCCN(CCCCCCCC(=O)OC(CCCCCCCC)CCCCCCCC)CCCNC(=O)C(C)OC] |
| I 268 | 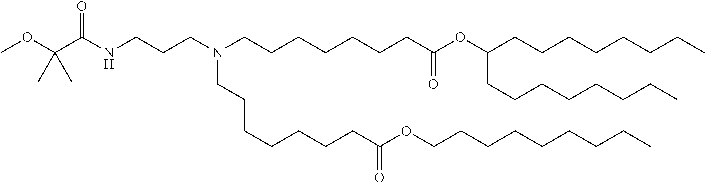 [SMILES: CCCCCCCCCOC(=O)CCCCCCCN(CCCCCCCC(=O)OC(CCCCCCCC)CCCCCCCC)CCCNC(=O)C(C)(C)OC] |
| I 269 | 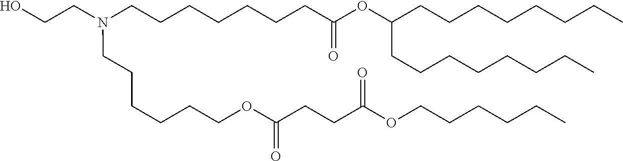 [SMILES: CCCCCCCCC(CCCCCCCC)OC(=O)CCCCCCCN(CCO)CCCCCCOC(=O)CCC(=O)OCCCCCC] |
| I 270 | 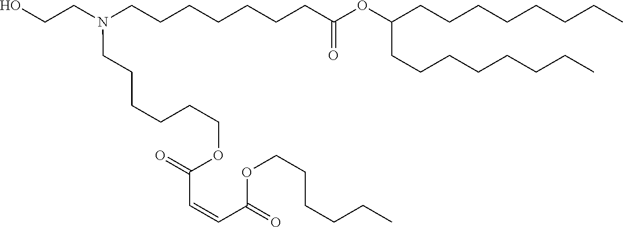 [SMILES: CCCCCCCCC(CCCCCCCC)OC(=O)CCCCCCCN(CCO)CCCCCCOC(=O)/C=C\C(=O)OCCCCCC] |
| I 271 | 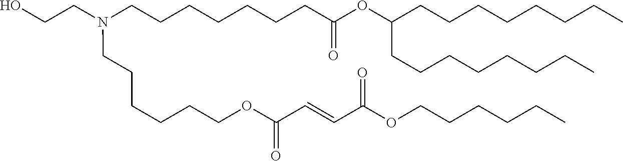 [SMILES: CCCCCCCCC(CCCCCCCC)OC(=O)CCCCCCCN(CCO)CCCCCCOC(=O)/C=C/C(=O)OCCCCCC] |
| I 272 | 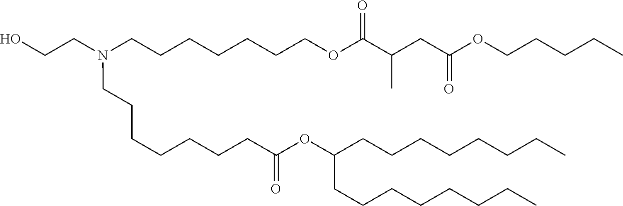 [SMILES: CCCCCCCCC(CCCCCCCC)OC(=O)CCCCCCCN(CCO)CCCCCCCOC(=O)C(C)CC(=O)OCCCCC] |
| I 273 | 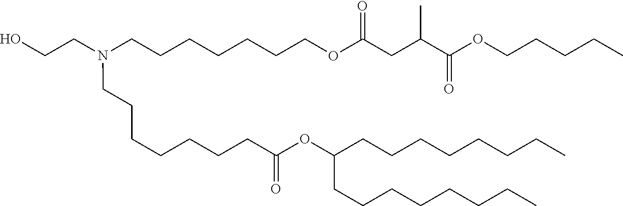 [SMILES: CCCCCCCCC(CCCCCCCC)OC(=O)CCCCCCCN(CCO)CCCCCCCOC(=O)CC(C)C(=O)OCCCCC] |
| I 274 | 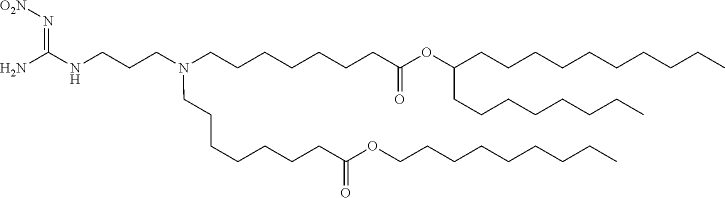 [SMILES: CCCCCCCCCCC(CCCCCCCC)OC(=O)CCCCCCCN(CCCCCCCC(=O)OCCCCCCCCC)CCCN/C(N)=N/[N+](=O)[O-]] |
| I 275 | 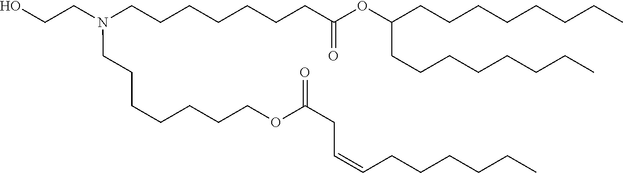 [SMILES: CCCCCC/C=C\CC(=O)OCCCCCCCN(CCO)CCCCCCCC(=O)OC(CCCCCCCC)CCCCCCCC] |
| I 276 | 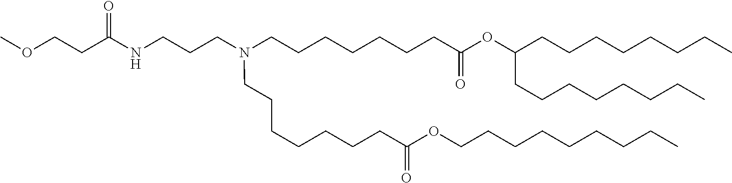 [SMILES: CCCCCCCCCOC(=O)CCCCCCCN(CCCCCCCC(=O)OC(CCCCCCCC)CCCCCCCC)CCCNC(=O)CCOC] |
| I 277 | 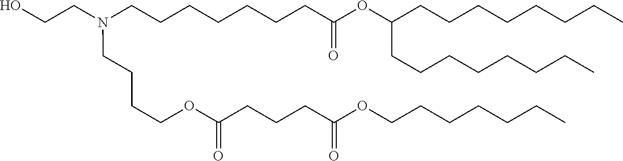 [SMILES: CCCCCCCCC(CCCCCCCC)OC(=O)CCCCCCCN(CCO)CCCCOC(=O)CCCC(=O)OCCCCCCC] |
| I 278 | 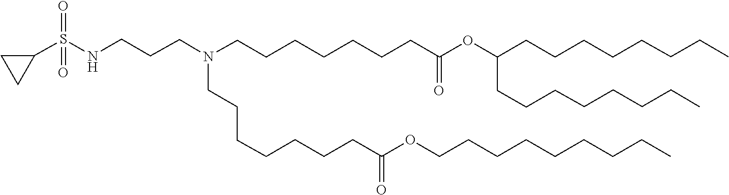 [SMILES: CCCCCCCCCOC(=O)CCCCCCCN(CCCCCCCC(=O)OC(CCCCCCCC)CCCCCCCC)CCCNS(=O)(=O)C1CC1] |
| I 279 | 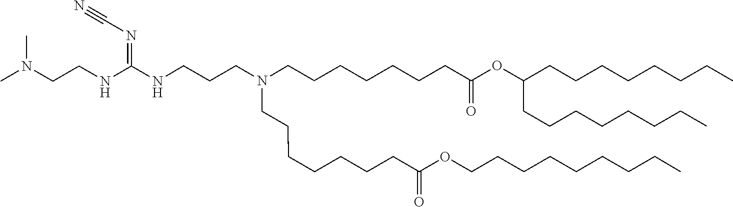 [SMILES: CCCCCCCCCOC(=O)CCCCCCCN(CCCCCCCC(=O)OC(CCCCCCCC)CCCCCCCC)CCCN/C(=N/C#N)NCCN(C)C] |
| I 280 | 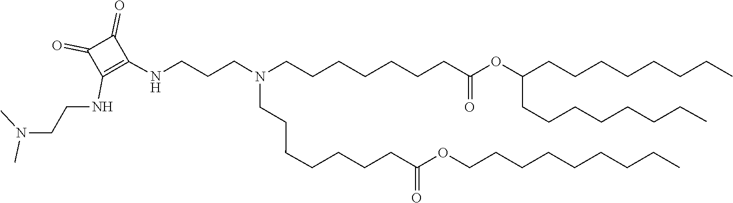 [SMILES: CCCCCCCCCOC(=O)CCCCCCCN(CCCCCCCC(=O)OC(CCCCCCCC)CCCCCCCC)CCCNc1c(NCCN(C)C)c(=O)c1=O] |
| I 281 | 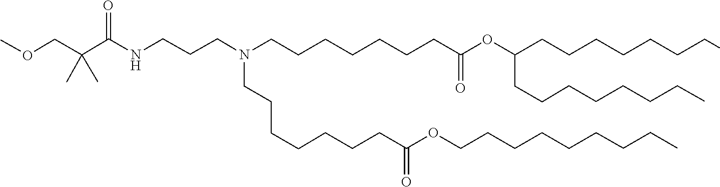 [SMILES: CCCCCCCCCOC(=O)CCCCCCCN(CCCCCCCC(=O)OC(CCCCCCCC)CCCCCCCC)CCCNC(=O)C(C)(C)COC] |
| I 282 | 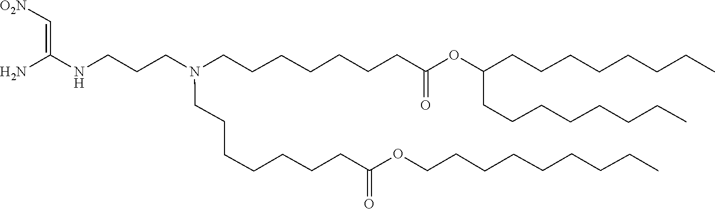 [SMILES: CCCCCCCCCOC(=O)CCCCCCCN(CCCCCCCC(=O)OC(CCCCCCCC)CCCCCCCC)CCCN/C(N)=C/[N+](=O)[O-]] |
| I 283 | 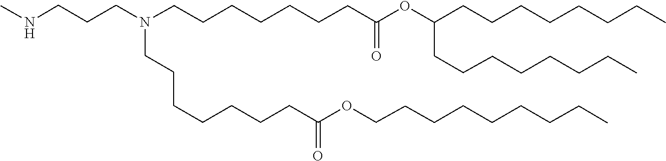 [SMILES: CCCCCCCCCOC(=O)CCCCCCCN(CCCCCCCC(=O)OC(CCCCCCCC)CCCCCCCC)CCCNC] |
| I 284 | 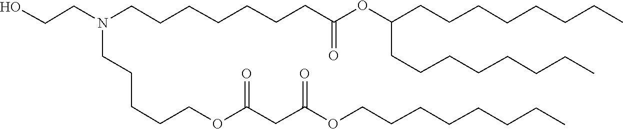 [SMILES: CCCCCCCCOC(=O)CC(=O)OCCCCCN(CCO)CCCCCCCC(=O)OC(CCCCCCCC)CCCCCCCC] |
| I 285 | 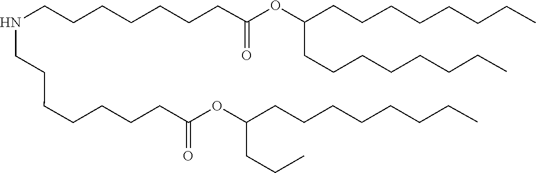 [SMILES: CCCCCCCCC(CCC)OC(=O)CCCCCCCNCCCCCCCC(=O)OC(CCCCCCCC)CCCCCCCC] |
| I 286 | 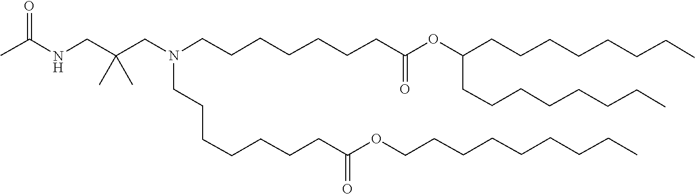 [SMILES: CCCCCCCCCOC(=O)CCCCCCCN(CCCCCCCC(=O)OC(CCCCCCCC)CCCCCCCC)CC(C)(C)CNC(C)=O] |
| I 287 | 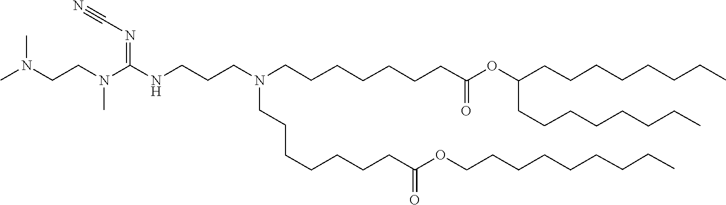 [SMILES: CCCCCCCCCOC(=O)CCCCCCCN(CCCCCCCC(=O)OC(CCCCCCCC)CCCCCCCC)CCCN/C(=N/C#N)N(C)CCN(C)C] |
| I 288 | 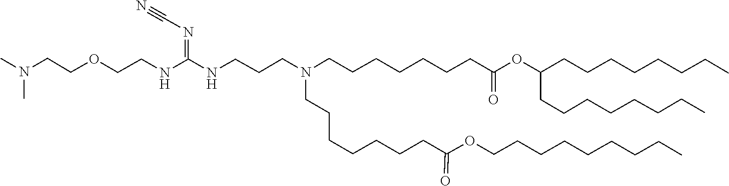 [SMILES: CCCCCCCCCOC(=O)CCCCCCCN(CCCCCCCC(=O)OC(CCCCCCCC)CCCCCCCC)CCCN/C(=N/C#N)NCCOCCN(C)C] |
| I 289 | 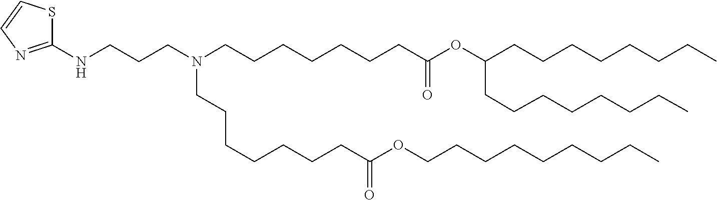 [SMILES: CCCCCCCCCOC(=O)CCCCCCCN(CCCCCCCC(=O)OC(CCCCCCCC)CCCCCCCC)CCCNc1nccs1] |
| I 290 | 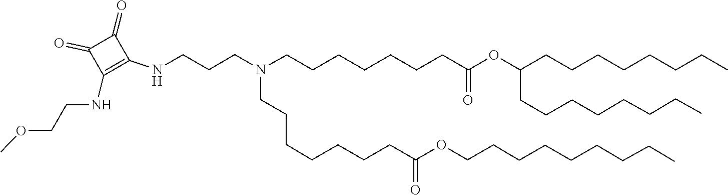 [SMILES: CCCCCCCCCOC(=O)CCCCCCCN(CCCCCCCC(=O)OC(CCCCCCCC)CCCCCCCC)CCCNc1c(NCCOC)c(=O)c1=O] |
| I 291 | 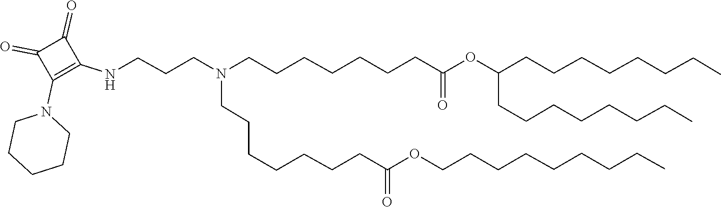 [SMILES: CCCCCCCCCOC(=O)CCCCCCCN(CCCCCCCC(=O)OC(CCCCCCCC)CCCCCCCC)CCCNc1c(N2CCCCC2)c(=O)c1=O] |
| I 292 | 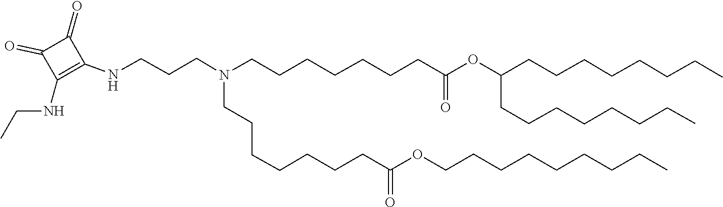 [SMILES: CCCCCCCCCOC(=O)CCCCCCCN(CCCCCCCC(=O)OC(CCCCCCCC)CCCCCCCC)CCCNc1c(NCC)c(=O)c1=O] |
| I 293 | 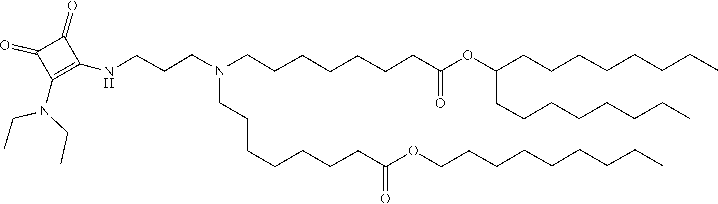 [SMILES: CCCCCCCCCOC(=O)CCCCCCCN(CCCCCCCC(=O)OC(CCCCCCCC)CCCCCCCC)CCCNc1c(N(CC)CC)c(=O)c1=O] |
| I 294 | 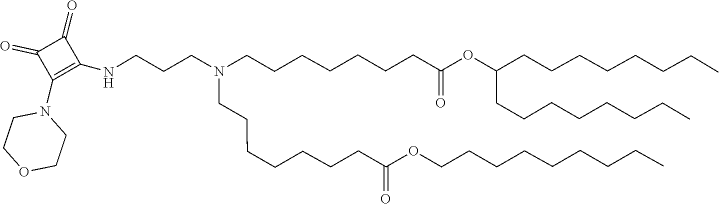 [SMILES: CCCCCCCCCOC(=O)CCCCCCCN(CCCCCCCC(=O)OC(CCCCCCCC)CCCCCCCC)CCCNc1c(N2CCOCC2)c(=O)c1=O] |
| I 295 | 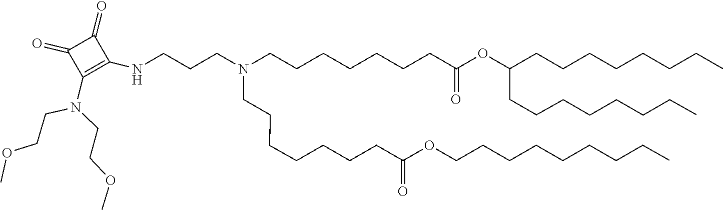 [SMILES: CCCCCCCCCOC(=O)CCCCCCCN(CCCCCCCC(=O)OC(CCCCCCCC)CCCCCCCC)CCCNc1c(N(CCOC)CCOC)c(=O)c1=O] |
| I 296 | 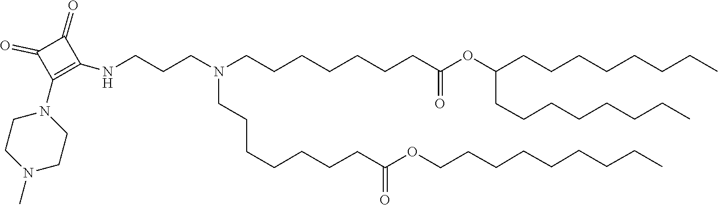 [SMILES: CCCCCCCCCOC(=O)CCCCCCCN(CCCCCCCC(=O)OC(CCCCCCCC)CCCCCCCC)CCCNc1c(N2CCN(C)CC2)c(=O)c1=O] |
| I 297 | 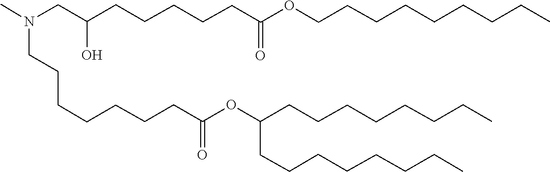 [SMILES: CCCCCCCCCOC(=O)CCCCCC(O)CN(C)CCCCCCCC(=O)OC(CCCCCCCC)CCCCCCCC] |
| I 298 | 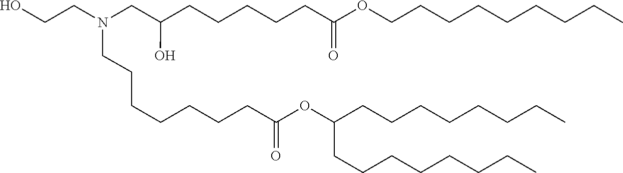 [SMILES: CCCCCCCCCOC(=O)CCCCCC(O)CN(CCO)CCCCCCCC(=O)OC(CCCCCCCC)CCCCCCCC] |
| I 299 | 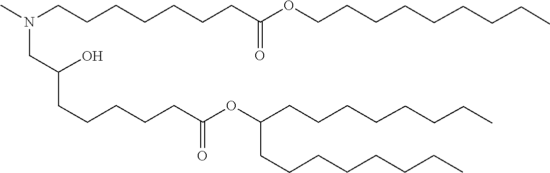 [SMILES: CCCCCCCCCOC(=O)CCCCCCCN(C)CC(O)CCCCCC(=O)OC(CCCCCCCC)CCCCCCCC] |
| I 300 | 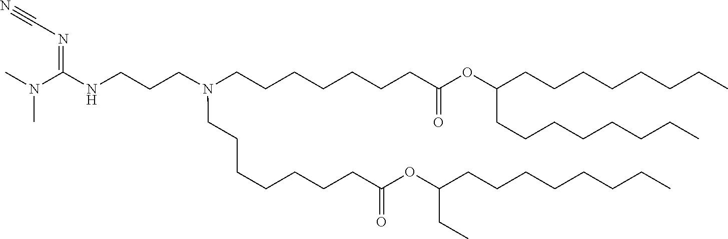 [SMILES: CCCCCCCCC(CC)OC(=O)CCCCCCCN(CCCCCCCC(=O)OC(CCCCCCCC)CCCCCCCC)CCCN/C(=N/C#N)N(C)C] |
| I 301 | 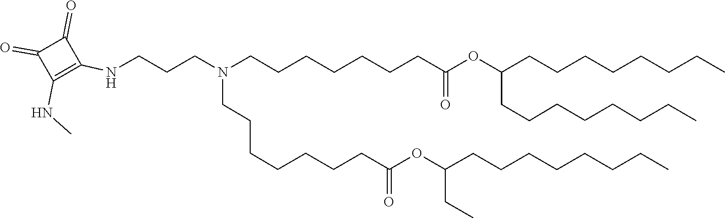 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| I 302 | 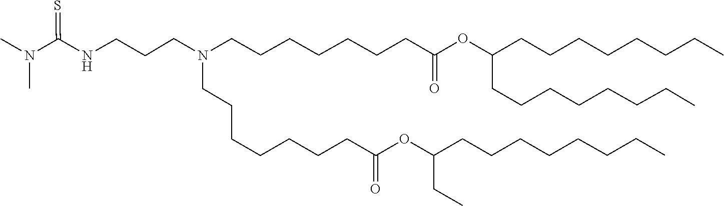 [SMILES: CCCCCCCCC(CC)OC(=O)CCCCCCCN(CCCCCCCC(=O)OC(CCCCCCCC)CCCCCCCC)CCCNC(=S)N(C)C] |
| I 303 | 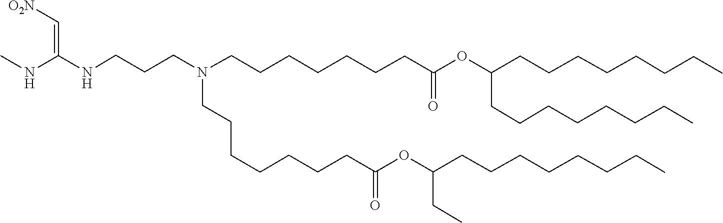 [SMILES: CCCCCCCCC(CC)OC(=O)CCCCCCCN(CCCCCCCC(=O)OC(CCCCCCCC)CCCCCCCC)CCCN/C(=C/[N+](=O)[O-])NC] |
| I 304 | 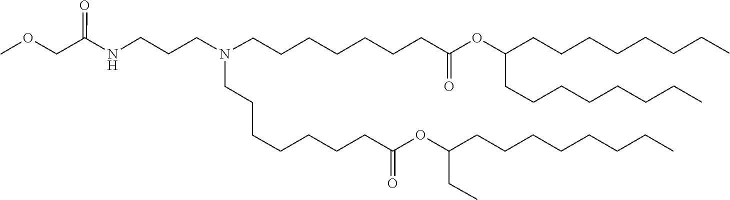 [SMILES: CCCCCCCCC(CC)OC(=O)CCCCCCCN(CCCCCCCC(=O)OC(CCCCCCCC)CCCCCCCC)CCCNC(=O)COC] |
| I 305 | 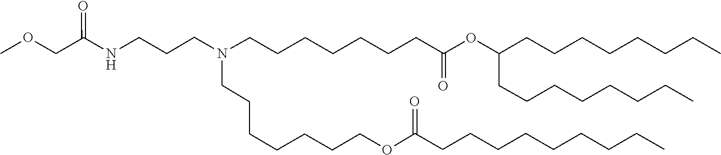 [SMILES: CCCCCCCCCC(=O)OCCCCCCCN(CCCCCCCC(=O)OC(CCCCCCCC)CCCCCCCC)CCCNC(=O)COC] |
| I 306 | 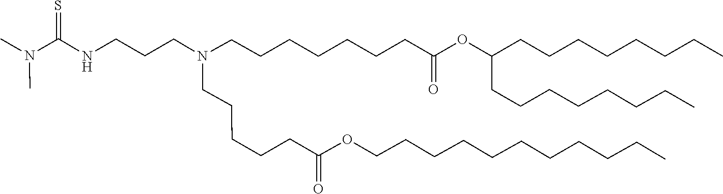 [SMILES: CCCCCCCCCCCOC(=O)CCCCCN(CCCCCCCC(=O)OC(CCCCCCCC)CCCCCCCC)CCCNC(=S)N(C)C] |
| I 307 | 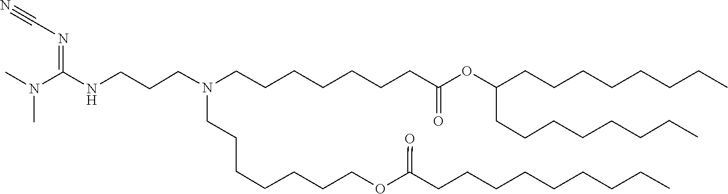 [SMILES: CCCCCCCCCC(=O)OCCCCCCCN(CCCCCCCC(=O)OC(CCCCCCCC)CCCCCCCC)CCCN/C(=N/C#N)N(C)C] |
| I 308 | 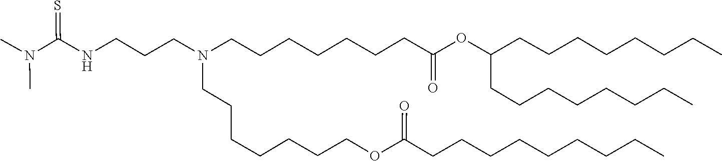 [SMILES: CCCCCCCCCC(=O)OCCCCCCCN(CCCCCCCC(=O)OC(CCCCCCCC)CCCCCCCC)CCCNC(=S)N(C)C] |
| I 309 | 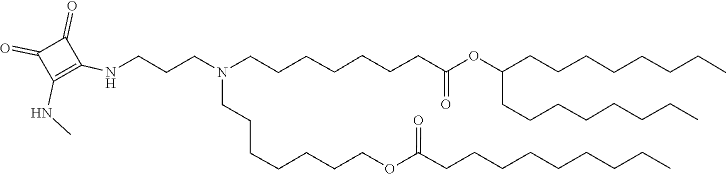 [SMILES: CCCCCCCCCC(=O)OCCCCCCCN(CCCCCCCC(=O)OC(CCCCCCCC)CCCCCCCC)CCCNc1c(NC)c(=O)c1=O] |
| I 310 | 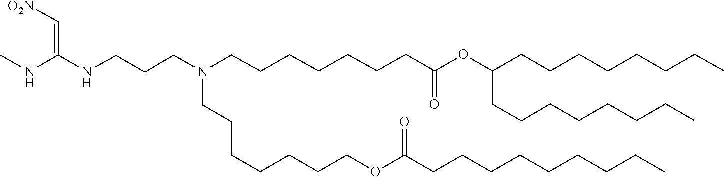 [SMILES: CCCCCCCCCC(=O)OCCCCCCCN(CCCCCCCC(=O)OC(CCCCCCCC)CCCCCCCC)CCCN/C(=C/[N+](=O)[O-])NC] |
| I 311 | 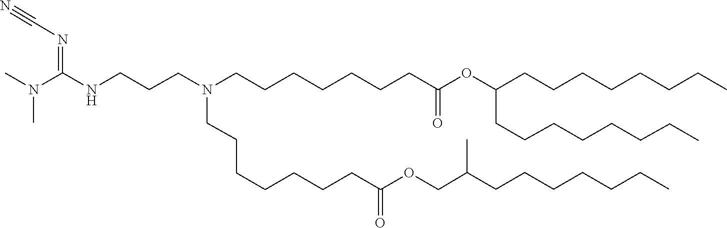 [SMILES: CCCCCCCCC(CCCCCCCC)OC(=O)CCCCCCCN(CCCCCCCC(=O)OCC(C)CCCCCCC)CCCN/C(=N/C#N)N(C)C] |
| I 312 | 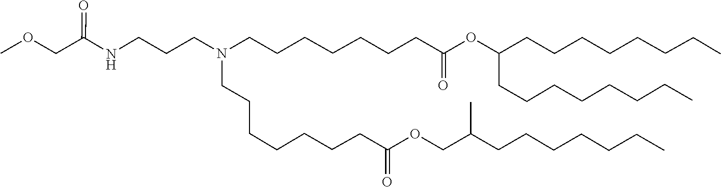 [SMILES: CCCCCCCCC(CCCCCCCC)OC(=O)CCCCCCCN(CCCCCCCC(=O)OCC(C)CCCCCCC)CCCNC(=O)COC] |
| I 313 | 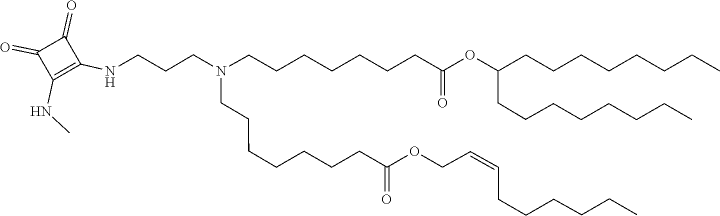 [SMILES: CCCCCC/C=C\COC(=O)CCCCCCCN(CCCCCCCC(=O)OC(CCCCCCCC)CCCCCCCC)CCCNc1c(NC)c(=O)c1=O] |
| I 314 | 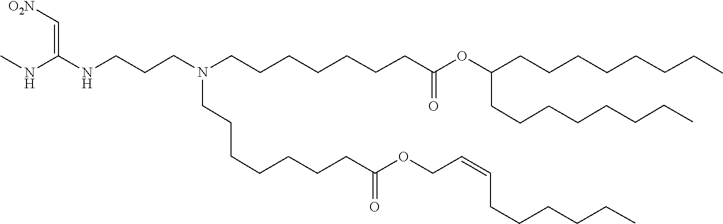 [SMILES: CCCCCC/C=C\COC(=O)CCCCCCCN(CCCCCCCC(=O)OC(CCCCCCCC)CCCCCCCC)CCCN/C(=C/[N+](=O)[O-])NC] |
| I 315 | 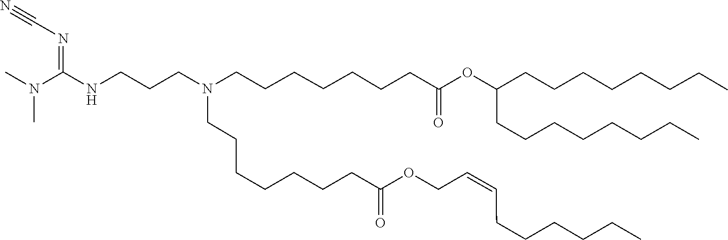 [SMILES: CCCCCC/C=C\COC(=O)CCCCCCCN(CCCCCCCC(=O)OC(CCCCCCCC)CCCCCCCC)CCCN/C(=N/C#N)N(C)C] |
| I 316 | 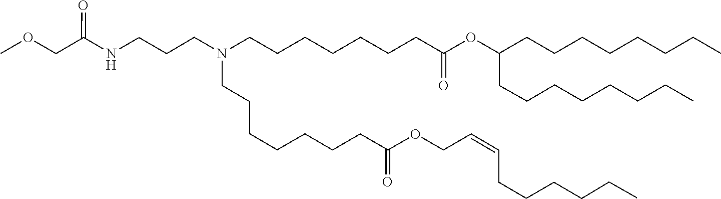 [SMILES: CCCCCC/C=C\COC(=O)CCCCCCCN(CCCCCCCC(=O)OC(CCCCCCCC)CCCCCCCC)CCCNC(=O)COC] |
| I 317 | 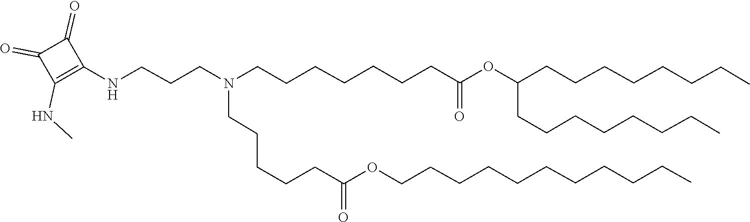 [SMILES: CCCCCCCCCCCOC(=O)CCCCCN(CCCCCCCC(=O)OC(CCCCCCCC)CCCCCCCC)CCCNc1c(NC)c(=O)c1=O] |
| I 318 | 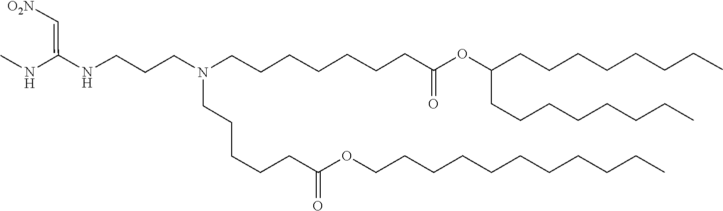 [SMILES: CCCCCCCCCCCOC(=O)CCCCCN(CCCCCCCC(=O)OC(CCCCCCCC)CCCCCCCC)CCCN/C(=C/[N+](=O)[O-])NC] |
| I 319 | 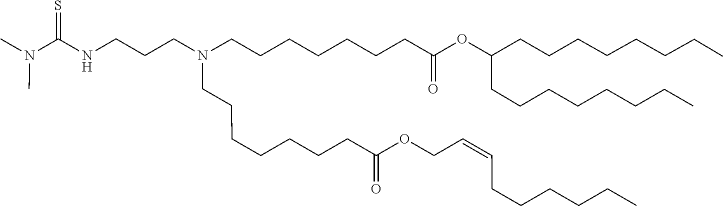 [SMILES: CCCCCC/C=C\COC(=O)CCCCCCCN(CCCCCCCC(=O)OC(CCCCCCCC)CCCCCCCC)CCCNC(=S)N(C)C] |
| I 320 | 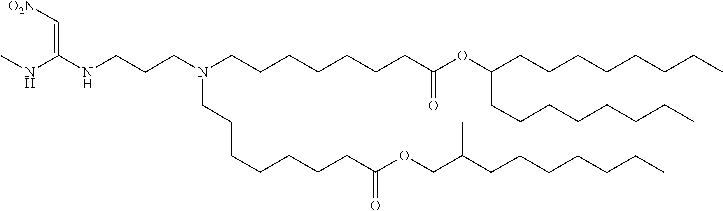 [SMILES: CCCCCCCCC(CCCCCCCC)OC(=O)CCCCCCCN(CCCCCCCC(=O)OCC(C)CCCCCCC)CCCN/C(=C/[N+](=O)[O-])NC] |
| I 321 | 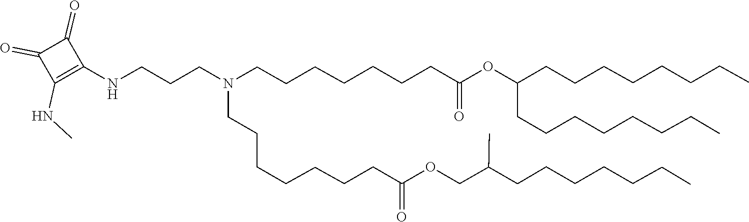 [SMILES: CCCCCCCCC(CCCCCCCC)OC(=O)CCCCCCCN(CCCCCCCC(=O)OCC(C)CCCCCCC)CCCNc1c(NC)c(=O)c1=O] |
| I 322 | 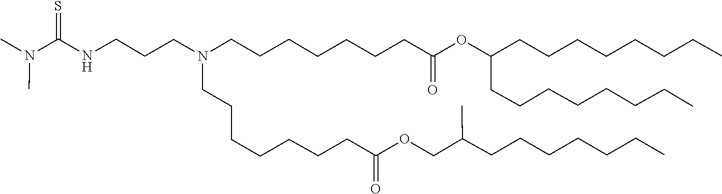 [SMILES: CCCCCCCCC(CCCCCCCC)OC(=O)CCCCCCCN(CCCCCCCC(=O)OCC(C)CCCCCCC)CCCNC(=S)N(C)C] |
| I 323 | 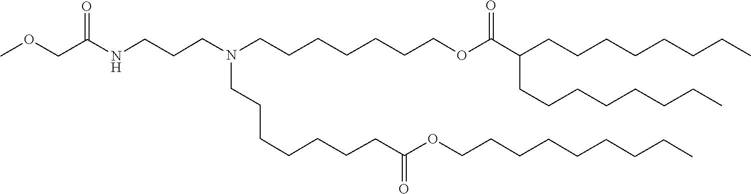 [SMILES: CCCCCCCCCOC(=O)CCCCCCCN(CCCCCCCOC(=O)C(CCCCCCCC)CCCCCCCC)CCCNC(=O)COC] |
| I 324 | 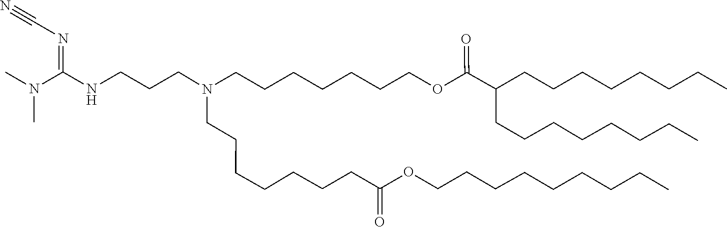 [SMILES: CCCCCCCCCOC(=O)CCCCCCCN(CCCCCCCOC(=O)C(CCCCCCCC)CCCCCCCC)CCCN/C(=N/C#N)N(C)C] |
| I 325 | 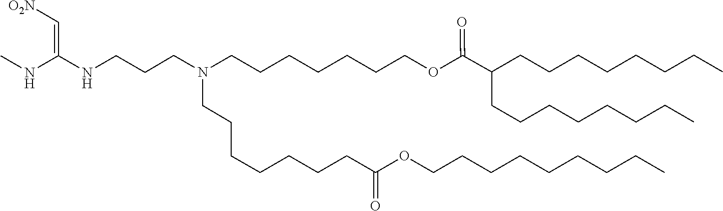 [SMILES: CCCCCCCCCOC(=O)CCCCCCCN(CCCCCCCOC(=O)C(CCCCCCCC)CCCCCCCC)CCCN/C(=C/[N+](=O)[O-])NC] |
| I 326 | 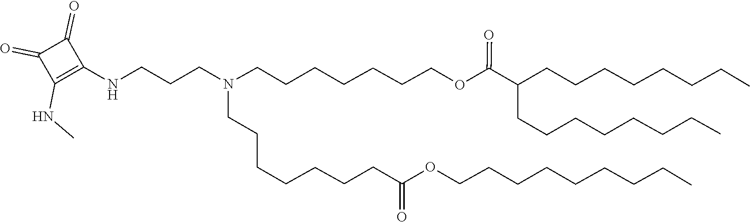 [SMILES: CCCCCCCCCOC(=O)CCCCCCCN(CCCCCCCOC(=O)C(CCCCCCCC)CCCCCCCC)CCCNc1c(NC)c(=O)c1=O] |
| I 262 | 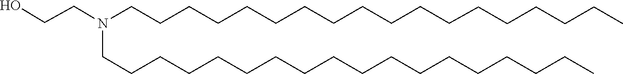 [SMILES: CCCCCCCCCCCCCCCCCCN(CCO)CCCCCCCCCCCCCCCCCC] |
| I 263 | 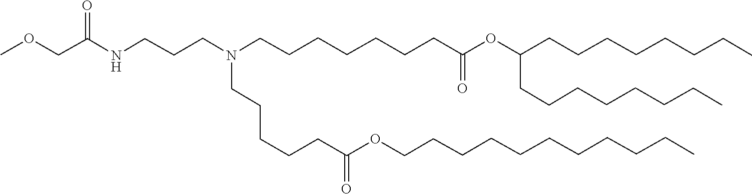 [SMILES: CCCCCCCCCCCOC(=O)CCCCCN(CCCCCCCC(=O)OC(CCCCCCCC)CCCCCCCC)CCCNC(=O)COC] |
| I 264 | 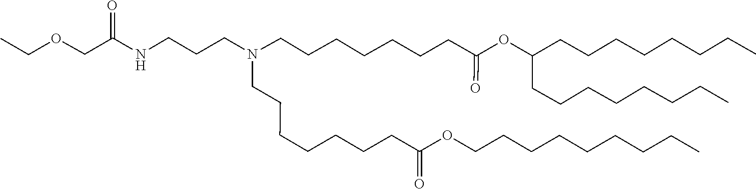 [SMILES: CCCCCCCCCOC(=O)CCCCCCCN(CCCCCCCC(=O)OC(CCCCCCCC)CCCCCCCC)CCCNC(=O)COCC] |
| I 265 | 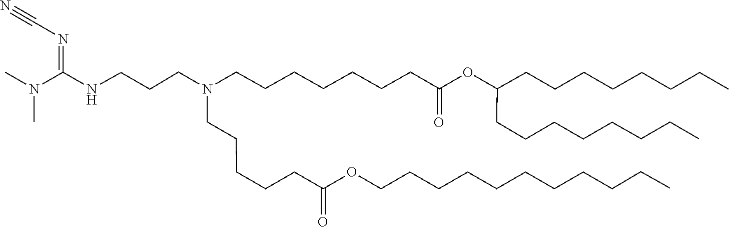 [SMILES: CCCCCCCCCCCOC(=O)CCCCCN(CCCCCCCC(=O)OC(CCCCCCCC)CCCCCCCC)CCCN/C(=N/C#N)N(C)C] |
| I 266 | 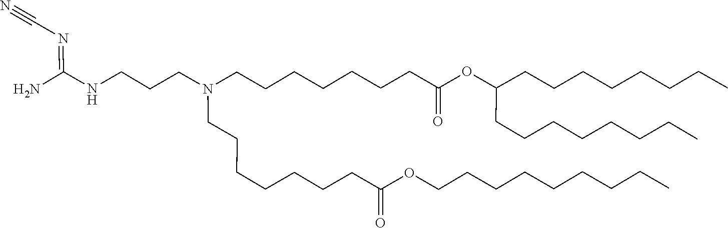 [SMILES: CCCCCCCCCOC(=O)CCCCCCCN(CCCCCCCC(=O)OC(CCCCCCCC)CCCCCCCC)CCCN/C(N)=N/C#N] |
| I 267 | 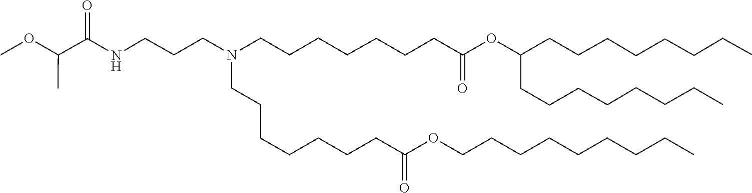 [SMILES: CCCCCCCCCOC(=O)CCCCCCCN(CCCCCCCC(=O)OC(CCCCCCCC)CCCCCCCC)CCCNC(=O)C(C)OC] |
| I 268 | 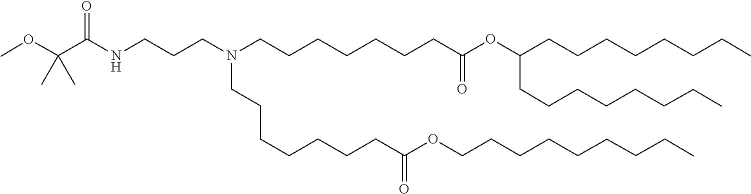 [SMILES: CCCCCCCCCOC(=O)CCCCCCCN(CCCCCCCC(=O)OC(CCCCCCCC)CCCCCCCC)CCCNC(=O)C(C)(C)OC] |
| I 269 | 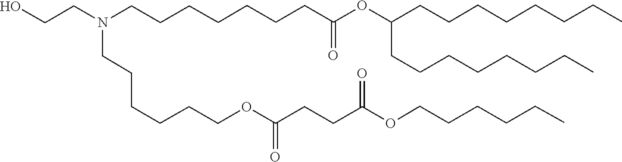 [SMILES: CCCCCCCCC(CCCCCCCC)OC(=O)CCCCCCCN(CCO)CCCCCCOC(=O)CCC(=O)OCCCCCC] |
| I 270 | 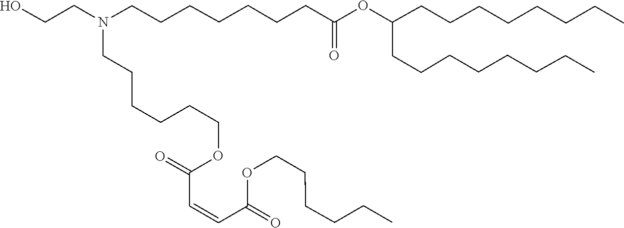 [SMILES: CCCCCCCCC(CCCCCCCC)OC(=O)CCCCCCCN(CCO)CCCCCCOC(=O)/C=C\C(=O)OCCCCCC] |
| I 271 | 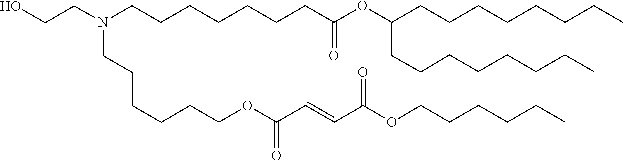 [SMILES: CCCCCCCCC(CCCCCCCC)OC(=O)CCCCCCCN(CCO)CCCCCCOC(=O)/C=C/C(=O)OCCCCCC] |
| I 272 | 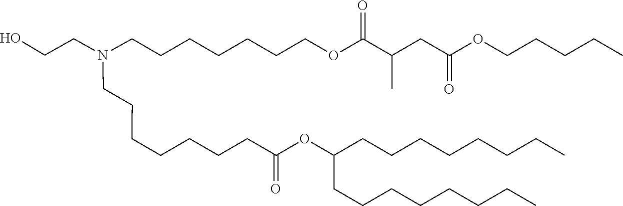 [SMILES: CCCCCCCCC(CCCCCCCC)OC(=O)CCCCCCCN(CCO)CCCCCCCOC(=O)C(C)CC(=O)OCCCCC] |
| I 273 | 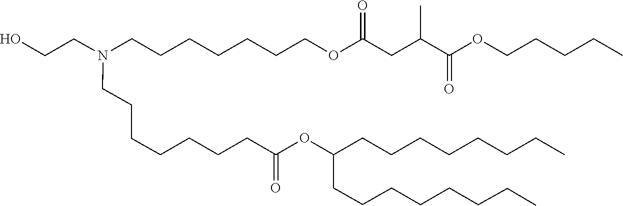 [SMILES: CCCCCCCCC(CCCCCCCC)OC(=O)CCCCCCCN(CCO)CCCCCCCOC(=O)CC(C)C(=O)OCCCCC] |
| I 274 | 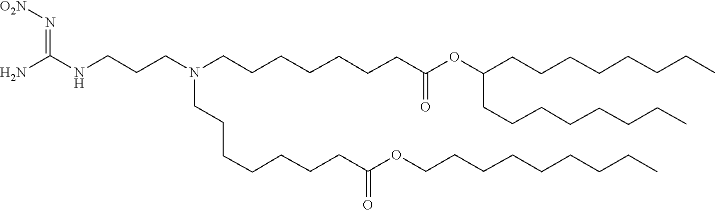 [SMILES: CCCCCCCCCOC(=O)CCCCCCCN(CCCCCCCC(=O)OC(CCCCCCCC)CCCCCCCC)CCCN/C(N)=N/[N+](=O)[O-]] |
| I 275 | 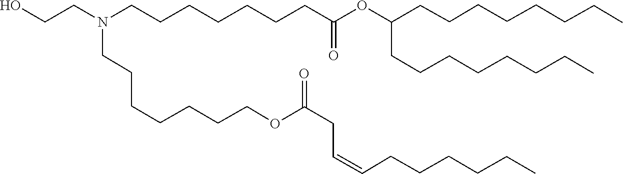 [SMILES: CCCCCC/C=C\CC(=O)OCCCCCCCN(CCO)CCCCCCCC(=O)OC(CCCCCCCC)CCCCCCCC] |
| I 276 | 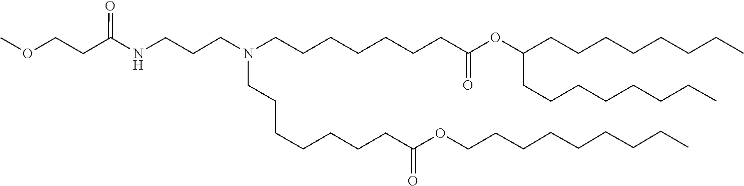 [SMILES: CCCCCCCCCOC(=O)CCCCCCCN(CCCCCCCC(=O)OC(CCCCCCCC)CCCCCCCC)CCCNC(=O)CCOC] |
| I 277 | 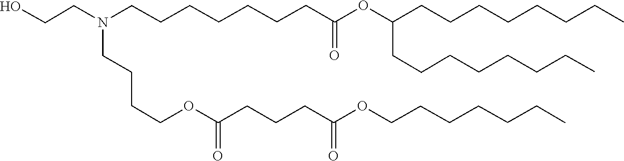 [SMILES: CCCCCCCCC(CCCCCCCC)OC(=O)CCCCCCCN(CCO)CCCCOC(=O)CCCC(=O)OCCCCCCC] |
| I 278 | 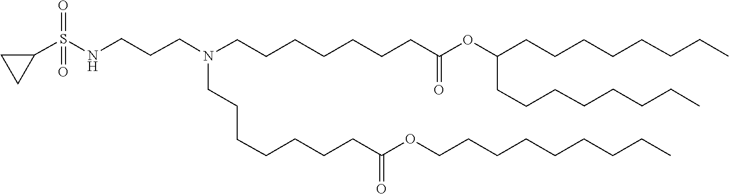 [SMILES: CCCCCCCCCOC(=O)CCCCCCCN(CCCCCCCC(=O)OC(CCCCCCCC)CCCCCCCC)CCCNS(=O)(=O)C1CC1] |
| I 279 | 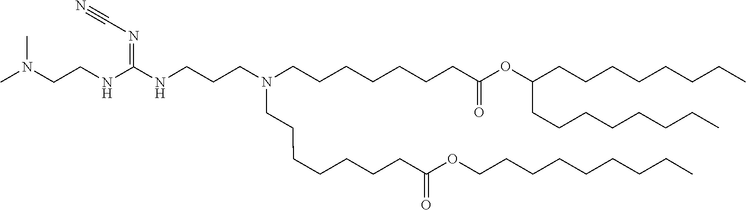 [SMILES: CCCCCCCCCOC(=O)CCCCCCCN(CCCCCCCC(=O)OC(CCCCCCCC)CCCCCCCC)CCCN/C(=N/C#N)NCCN(C)C] |
| I 280 | 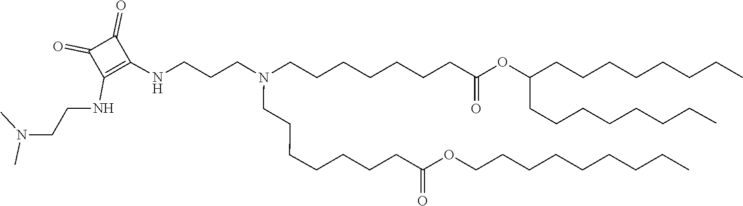 [SMILES: CCCCCCCCCOC(=O)CCCCCCCN(CCCCCCCC(=O)OC(CCCCCCCC)CCCCCCCC)CCCNc1c(NCCN(C)C)c(=O)c1=O] |
| I 281 | 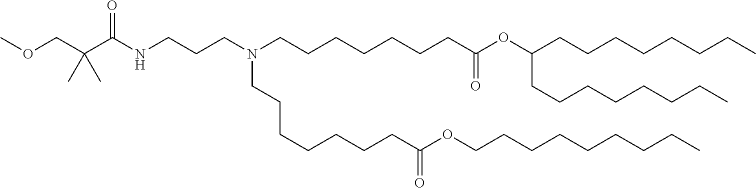 [SMILES: CCCCCCCCCOC(=O)CCCCCCCN(CCCCCCCC(=O)OC(CCCCCCCC)CCCCCCCC)CCCNC(=O)C(C)(C)COC] |
| I 282 | 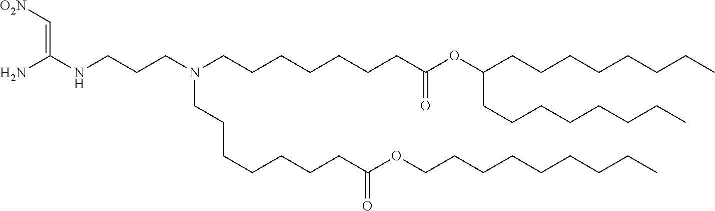 [SMILES: CCCCCCCCCOC(=O)CCCCCCCN(CCCCCCCC(=O)OC(CCCCCCCC)CCCCCCCC)CCCN/C(N)=C/[N+](=O)[O-]] |
| I 283 | 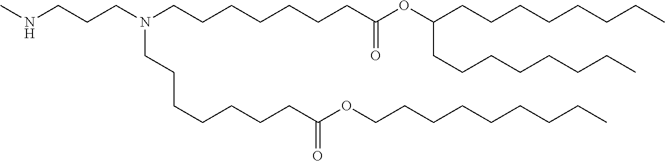 [SMILES: CCCCCCCCCOC(=O)CCCCCCCN(CCCCCCCC(=O)OC(CCCCCCCC)CCCCCCCC)CCCNC] |
| I 284 | 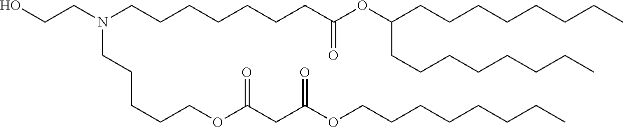 [SMILES: CCCCCCCCOC(=O)CC(=O)OCCCCCN(CCO)CCCCCCCC(=O)OC(CCCCCCCC)CCCCCCCC] |
| I 285 | 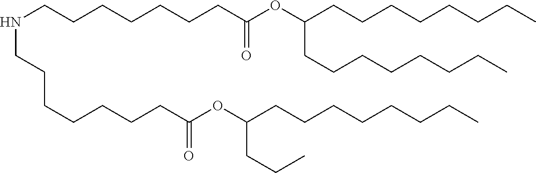 [SMILES: CCCCCCCCC(CCC)OC(=O)CCCCCCCNCCCCCCCC(=O)OC(CCCCCCCC)CCCCCCCC] |
| I 286 | 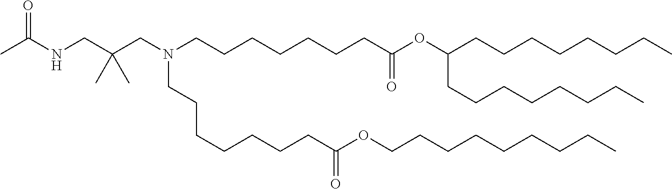 [SMILES: CCCCCCCCCOC(=O)CCCCCCCN(CCCCCCCC(=O)OC(CCCCCCCC)CCCCCCCC)CC(C)(C)CNC(C)=O] |
| I 287 | 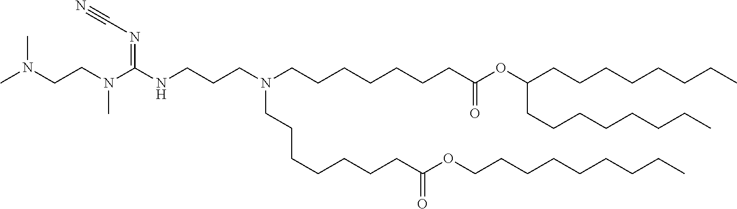 [SMILES: CCCCCCCCCOC(=O)CCCCCCCN(CCCCCCCC(=O)OC(CCCCCCCC)CCCCCCCC)CCCN/C(=N/C#N)N(C)CCN(C)C] |
| I 288 | 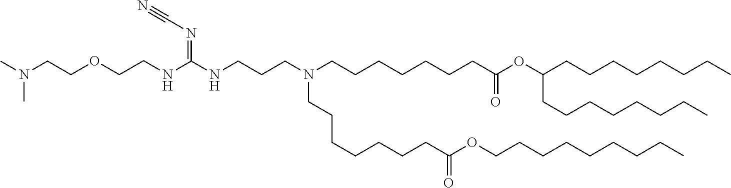 [SMILES: CCCCCCCCCOC(=O)CCCCCCCN(CCCCCCCC(=O)OC(CCCCCCCC)CCCCCCCC)CCCN/C(=N/C#N)NCCOCCN(C)C] |
| I 289 | 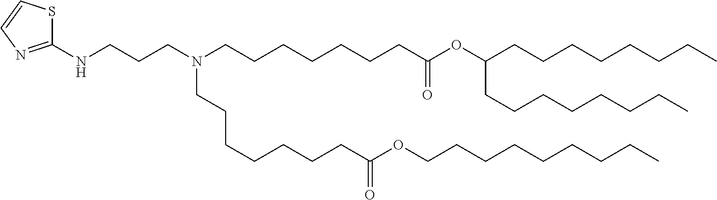 [SMILES: CCCCCCCCCOC(=O)CCCCCCCN(CCCCCCCC(=O)OC(CCCCCCCC)CCCCCCCC)CCCNc1nccs1] |
| I 290 | 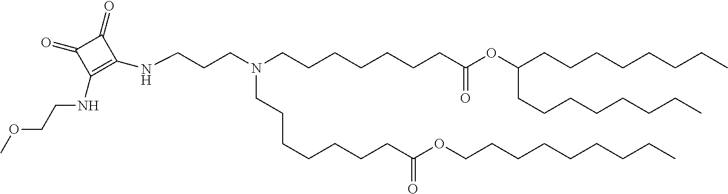 [SMILES: CCCCCCCCCOC(=O)CCCCCCCN(CCCCCCCC(=O)OC(CCCCCCCC)CCCCCCCC)CCCNc1c(NCCOC)c(=O)c1=O] |
| I 291 | 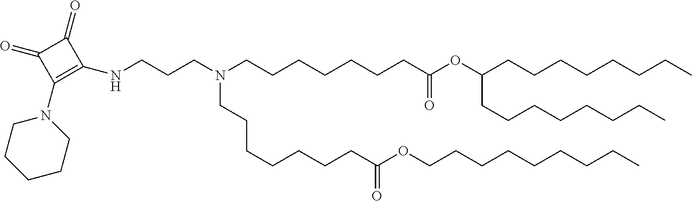 [SMILES: CCCCCCCCCOC(=O)CCCCCCCN(CCCCCCCC(=O)OC(CCCCCCCC)CCCCCCCC)CCCNc1c(N2CCCCC2)c(=O)c1=O] |
| I 292 | 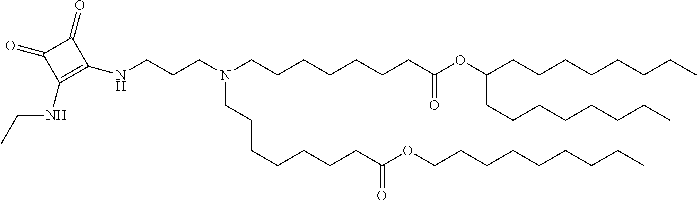 [SMILES: CCCCCCCCCOC(=O)CCCCCCCN(CCCCCCCC(=O)OC(CCCCCCCC)CCCCCCCC)CCCNc1c(NCC)c(=O)c1=O] |
| I 293 | 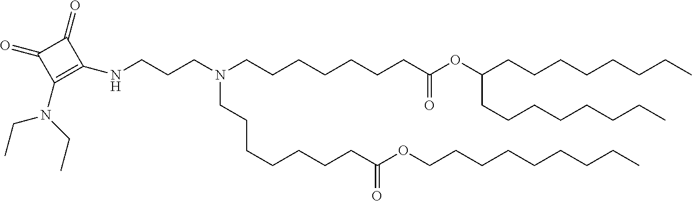 [SMILES: CCCCCCCCCOC(=O)CCCCCCCN(CCCCCCCC(=O)OC(CCCCCCCC)CCCCCCCC)CCCNc1c(N(CC)CC)c(=O)c1=O] |
| I 294 | 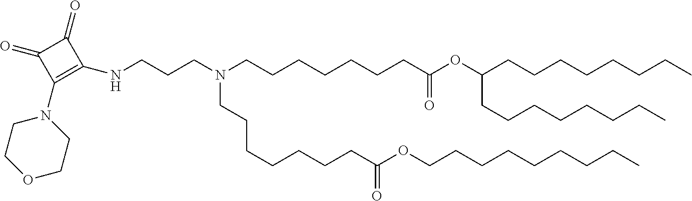 [SMILES: CCCCCCCCCOC(=O)CCCCCCCN(CCCCCCCC(=O)OC(CCCCCCCC)CCCCCCCC)CCCNc1c(N2CCOCC2)c(=O)c1=O] |
| I 295 | 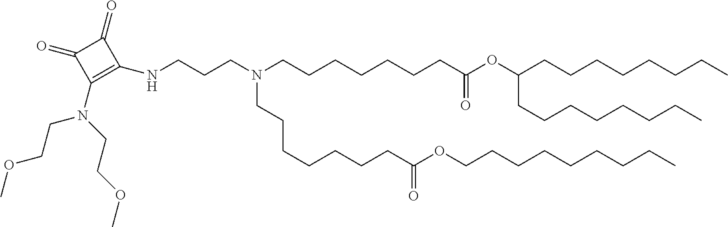 [SMILES: CCCCCCCCCOC(=O)CCCCCCCN(CCCCCCCC(=O)OC(CCCCCCCC)CCCCCCCC)CCCNc1c(N(CCOC)CCOC)c(=O)c1=O] |
| I 296 | 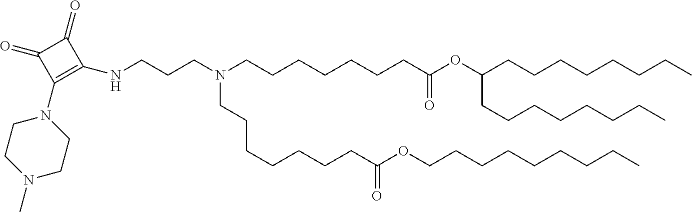 [SMILES: CCCCCCCCCOC(=O)CCCCCCCN(CCCCCCCC(=O)OC(CCCCCCCC)CCCCCCCC)CCCNc1c(N2CCN(C)CC2)c(=O)c1=O] |
| I 297 | 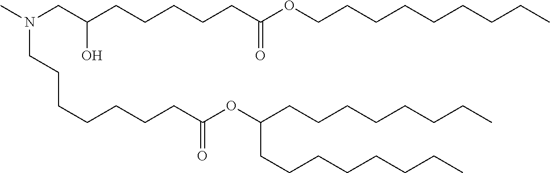 [SMILES: CCCCCCCCCOC(=O)CCCCCC(O)CN(C)CCCCCCCC(=O)OC(CCCCCCCC)CCCCCCCC] |
| I 298 | 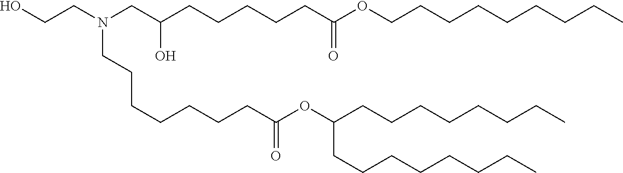 [SMILES: CCCCCCCCCOC(=O)CCCCCC(O)CN(CCO)CCCCCCCC(=O)OC(CCCCCCCC)CCCCCCCC] |
| I 299 | 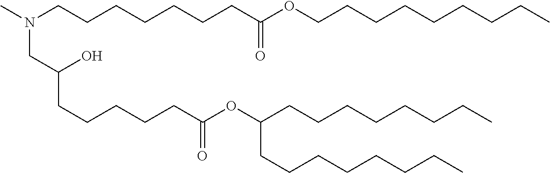 [SMILES: CCCCCCCCCOC(=O)CCCCCCCN(C)CC(O)CCCCCC(=O)OC(CCCCCCCC)CCCCCCCC] |
| I 300 | 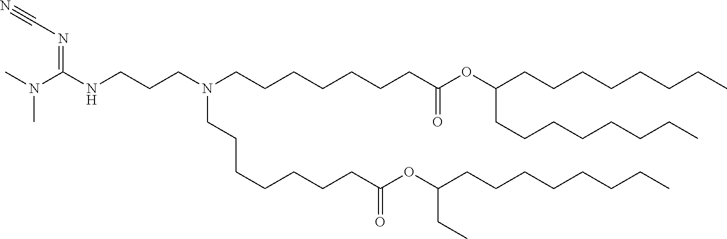 [SMILES: CCCCCCCCC(CC)OC(=O)CCCCCCCN(CCCCCCCC(=O)OC(CCCCCCCC)CCCCCCCC)CCCN/C(=N/C#N)N(C)C] |
| I 301 | 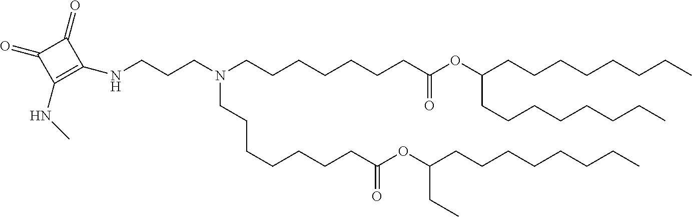 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| I 302 | 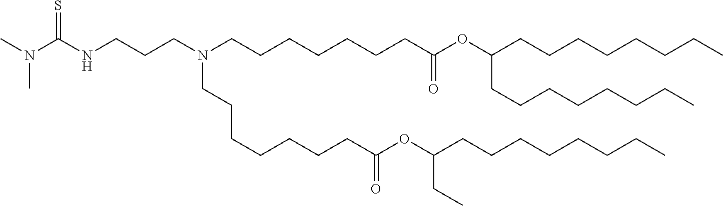 [SMILES: CCCCCCCCC(CC)OC(=O)CCCCCCCN(CCCCCCCC(=O)OC(CCCCCCCC)CCCCCCCC)CCCNC(=S)N(C)C] |
| I 303 | 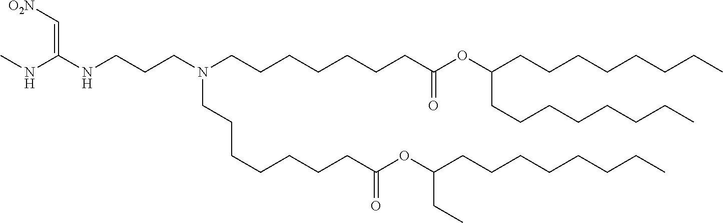 [SMILES: CCCCCCCCC(CC)OC(=O)CCCCCCCN(CCCCCCCC(=O)OC(CCCCCCCC)CCCCCCCC)CCCN/C(=C/[N+](=O)[O-])NC] |
| I 304 | 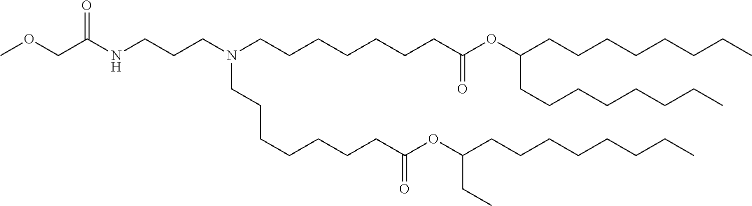 [SMILES: CCCCCCCCC(CC)OC(=O)CCCCCCCN(CCCCCCCC(=O)OC(CCCCCCCC)CCCCCCCC)CCCNC(=O)COC] |
| I 305 | 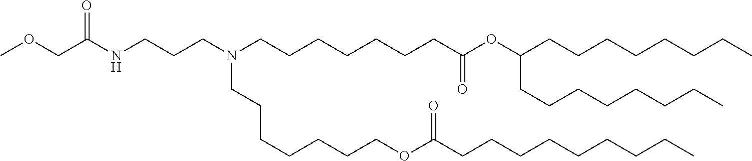 [SMILES: CCCCCCCCCC(=O)OCCCCCCCN(CCCCCCCC(=O)OC(CCCCCCCC)CCCCCCCC)CCCNC(=O)COC] |
| I 306 | 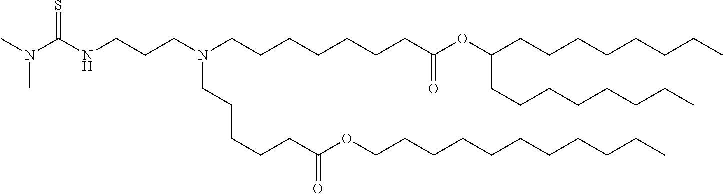 [SMILES: CCCCCCCCCCCOC(=O)CCCCCN(CCCCCCCC(=O)OC(CCCCCCCC)CCCCCCCC)CCCNC(=S)N(C)C] |
| I 307 | 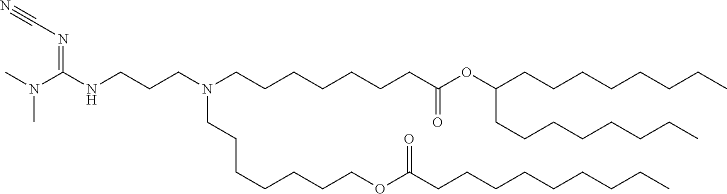 [SMILES: CCCCCCCCCC(=O)OCCCCCCCN(CCCCCCCC(=O)OC(CCCCCCCC)CCCCCCCC)CCCN/C(=N/C#N)N(C)C] |
| I 308 | 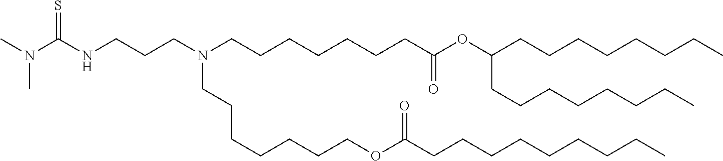 [SMILES: CCCCCCCCCC(=O)OCCCCCCCN(CCCCCCCC(=O)OC(CCCCCCCC)CCCCCCCC)CCCNC(=S)N(C)C] |
| I 309 | 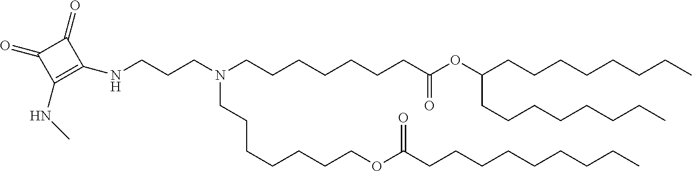 [SMILES: CCCCCCCCCC(=O)OCCCCCCCN(CCCCCCCC(=O)OC(CCCCCCCC)CCCCCCCC)CCCNc1c(NC)c(=O)c1=O] |
| I 310 | 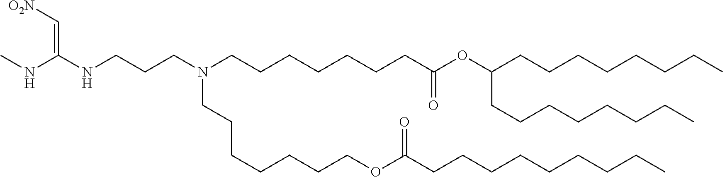 [SMILES: CCCCCCCCCC(=O)OCCCCCCCN(CCCCCCCC(=O)OC(CCCCCCCC)CCCCCCCC)CCCN/C(=C/[N+](=O)[O-])NC] |
| I 311 | 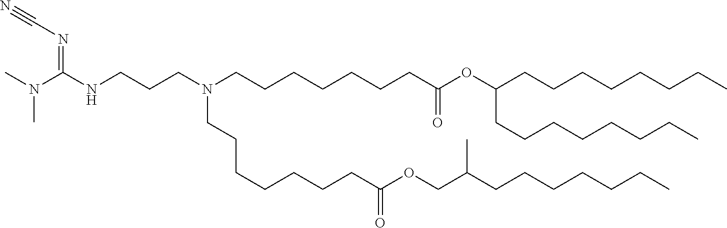 [SMILES: CCCCCCCCC(CCCCCCCC)OC(=O)CCCCCCCN(CCCCCCCC(=O)OCC(C)CCCCCCC)CCCN/C(=N/C#N)N(C)C] |
| I 312 | 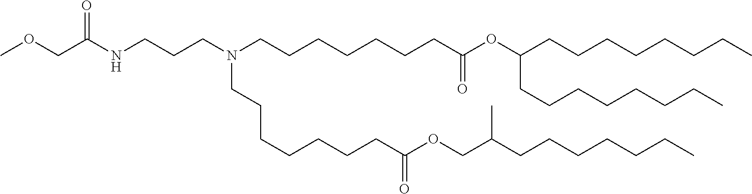 [SMILES: CCCCCCCCC(CCCCCCCC)OC(=O)CCCCCCCN(CCCCCCCC(=O)OCC(C)CCCCCCC)CCCNC(=O)COC] |
| I 313 | 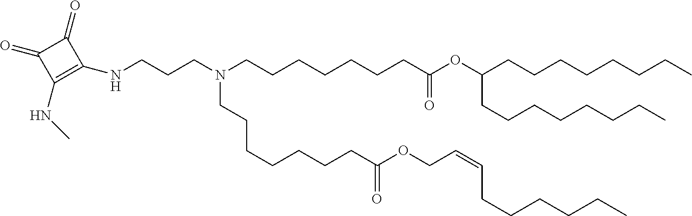 [SMILES: CCCCCC/C=C\COC(=O)CCCCCCCN(CCCCCCCC(=O)OC(CCCCCCCC)CCCCCCCC)CCCNc1c(NC)c(=O)c1=O] |
| I 314 | 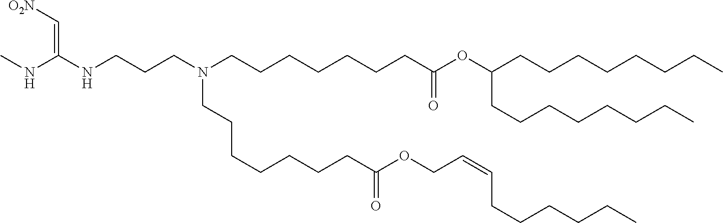 [SMILES: CCCCCC/C=C\COC(=O)CCCCCCCN(CCCCCCCC(=O)OC(CCCCCCCC)CCCCCCCC)CCCN/C(=C/[N+](=O)[O-])NC] |
| I 315 | 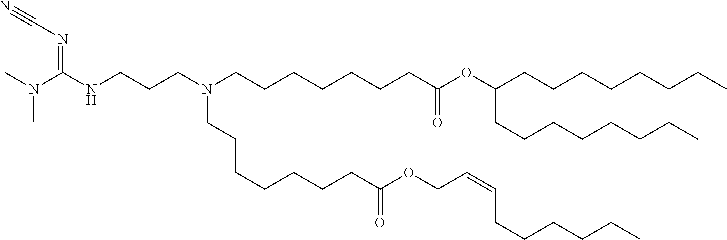 [SMILES: CCCCCC/C=C\COC(=O)CCCCCCCN(CCCCCCCC(=O)OC(CCCCCCCC)CCCCCCCC)CCCN/C(=N/C#N)N(C)C] |
| I 316 | 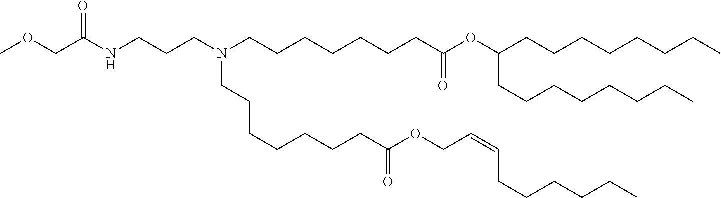 [SMILES: CCCCCC/C=C\COC(=O)CCCCCCCN(CCCCCCCC(=O)OC(CCCCCCCC)CCCCCCCC)CCCNC(=O)COC] |
| I 317 | 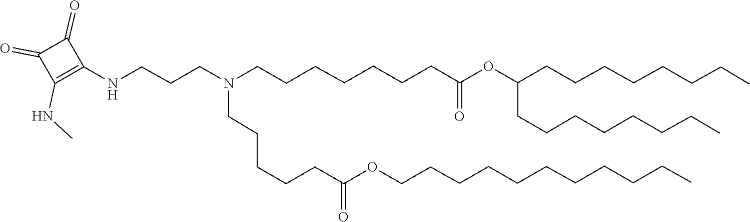 [SMILES: CCCCCCCCCCCOC(=O)CCCCCN(CCCCCCCC(=O)OC(CCCCCCCC)CCCCCCCC)CCCNc1c(NC)c(=O)c1=O] |
| I 318 | 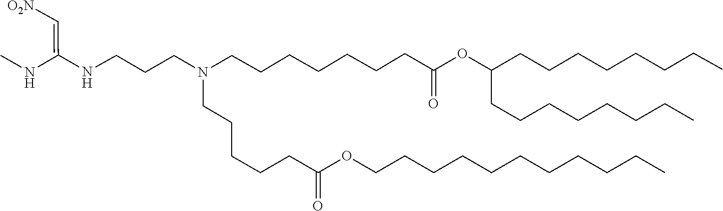 [SMILES: CCCCCCCCCCCOC(=O)CCCCCN(CCCCCCCC(=O)OC(CCCCCCCC)CCCCCCCC)CCCN/C(=C/[N+](=O)[O-])NC] |
| I 319 | 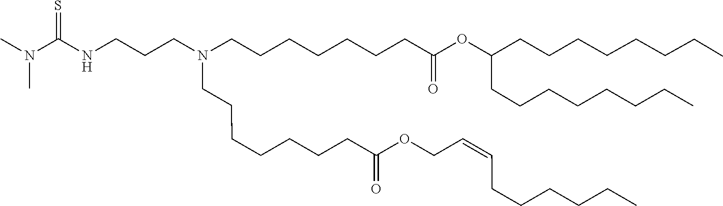 [SMILES: CCCCCC/C=C\COC(=O)CCCCCCCN(CCCCCCCC(=O)OC(CCCCCCCC)CCCCCCCC)CCCNC(=S)N(C)C] |
| I 320 | 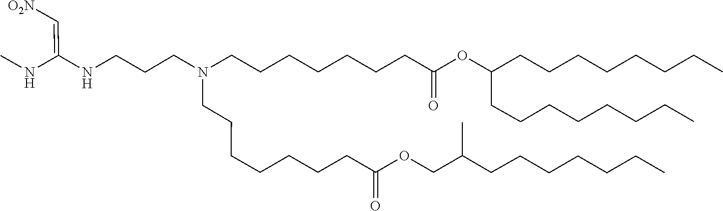 [SMILES: CCCCCCCCC(CCCCCCCC)OC(=O)CCCCCCCN(CCCCCCCC(=O)OCC(C)CCCCCCC)CCCN/C(=C/[N+](=O)[O-])NC] |
| I 321 | 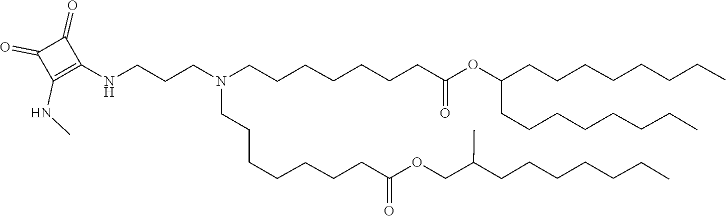 [SMILES: CCCCCCCCC(CCCCCCCC)OC(=O)CCCCCCCN(CCCCCCCC(=O)OCC(C)CCCCCCC)CCCNc1c(NC)c(=O)c1=O] |
| I 322 | 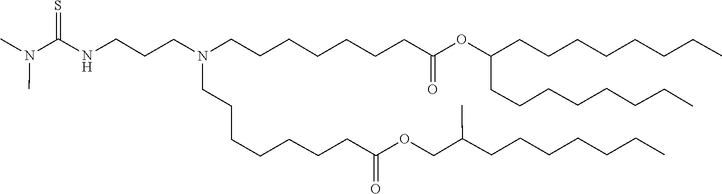 [SMILES: CCCCCCCCC(CCCCCCCC)OC(=O)CCCCCCCN(CCCCCCCC(=O)OCC(C)CCCCCCC)CCCNC(=S)N(C)C] |
| I 323 | 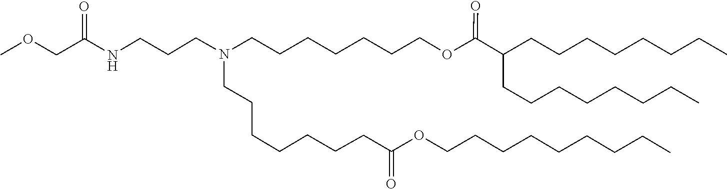 [SMILES: CCCCCCCCCOC(=O)CCCCCCCN(CCCCCCCOC(=O)C(CCCCCCCC)CCCCCCCC)CCCNC(=O)COC] |
| I 324 | 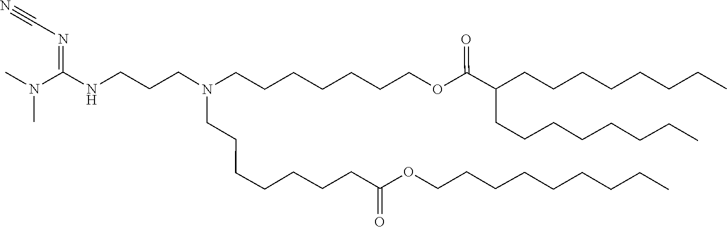 [SMILES: CCCCCCCCCOC(=O)CCCCCCCN(CCCCCCCOC(=O)C(CCCCCCCC)CCCCCCCC)CCCN/C(=N/C#N)N(C)C] |
| I 325 | 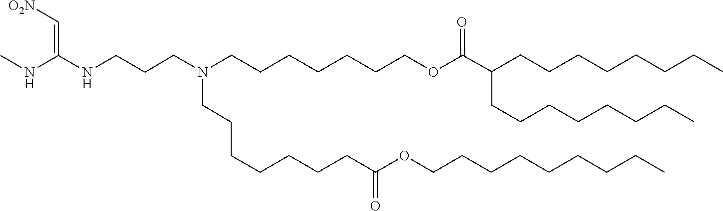 [SMILES: CCCCCCCCCOC(=O)CCCCCCCN(CCCCCCCOC(=O)C(CCCCCCCC)CCCCCCCC)CCCN/C(=C/[N+](=O)[O-])NC] |
| I 326 | 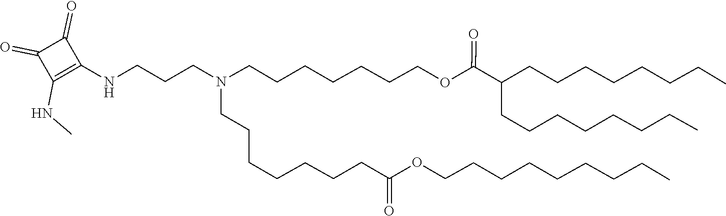 [SMILES: CCCCCCCCCOC(=O)CCCCCCCN(CCCCCCCOC(=O)C(CCCCCCCC)CCCCCCCC)CCCNc1c(NC)c(=O)c1=O] |
| I 327 | 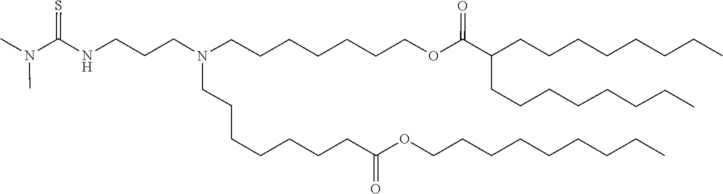 [SMILES: CCCCCCCCCOC(=O)CCCCCCCN(CCCCCCCOC(=O)C(CCCCCCCC)CCCCCCCC)CCCNC(=S)N(C)C] |
| I 328 | 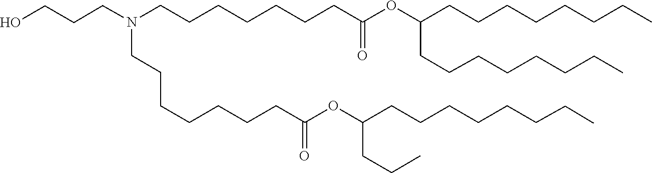 [SMILES: CCCCCCCCC(CCC)OC(=O)CCCCCCCN(CCCO)CCCCCCCC(=O)OC(CCCCCCCC)CCCCCCCC] |
| I 329 | 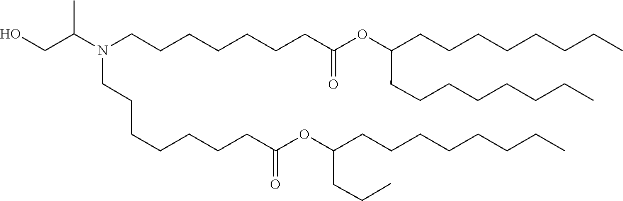 [SMILES: CCCCCCCCC(CCC)OC(=O)CCCCCCCN(CCCCCCCC(=O)OC(CCCCCCCC)CCCCCCCC)C(C)CO] |
| I 330 | 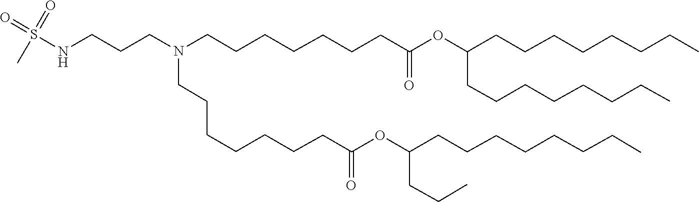 [SMILES: CCCCCCCCC(CCC)OC(=O)CCCCCCCN(CCCCCCCC(=O)OC(CCCCCCCC)CCCCCCCC)CCCNS(C)(=O)=O] |
| I 331 | 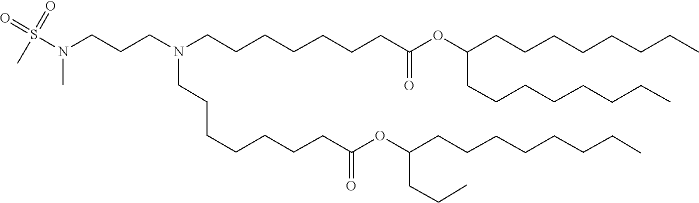 [SMILES: CCCCCCCCC(CCC)OC(=O)CCCCCCCN(CCCCCCCC(=O)OC(CCCCCCCC)CCCCCCCC)CCCN(C)S(C)(=O)=O] |
| I 332 | 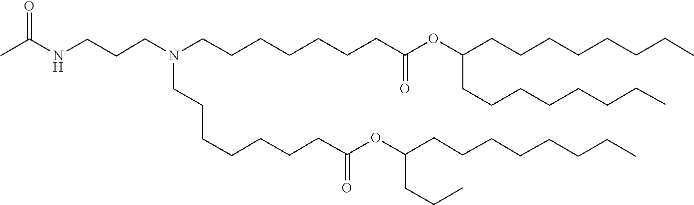 [SMILES: CCCCCCCCC(CCC)OC(=O)CCCCCCCN(CCCCCCCC(=O)OC(CCCCCCCC)CCCCCCCC)CCCNC(C)=O] |
| I 333 | 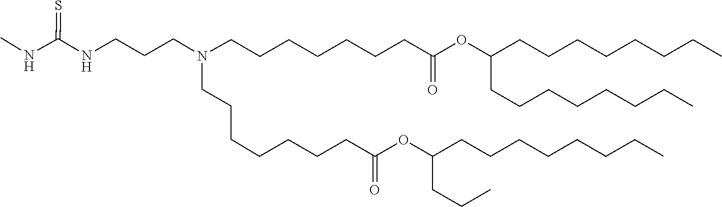 [SMILES: CCCCCCCCC(CCC)OC(=O)CCCCCCCN(CCCCCCCC(=O)OC(CCCCCCCC)CCCCCCCC)CCCNC(=S)NC] |
| I 334 | 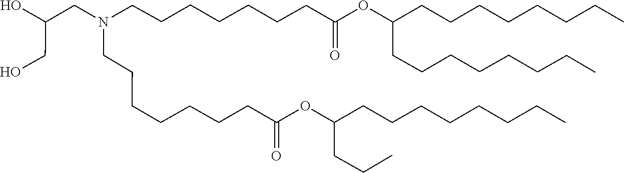 [SMILES: CCCCCCCCC(CCC)OC(=O)CCCCCCCN(CCCCCCCC(=O)OC(CCCCCCCC)CCCCCCCC)CC(O)CO] |
| I 335 | 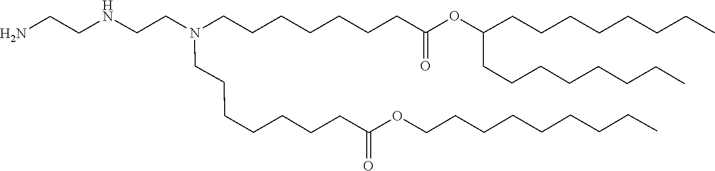 [SMILES: CCCCCCCCCOC(=O)CCCCCCCN(CCCCCCCC(=O)OC(CCCCCCCC)CCCCCCCC)CCNCCN] |
| I 336 | 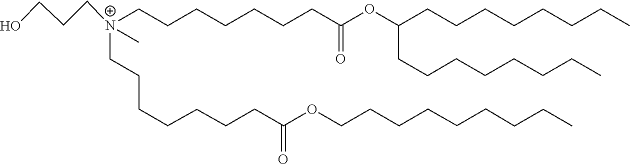 [SMILES: CCCCCCCCCOC(=O)CCCCCCC[N+](C)(CCCO)CCCCCCCC(=O)OC(CCCCCCCC)CCCCCCCC] |
| I 337 | 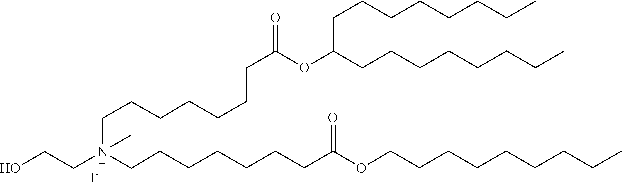 |
| I 338 | 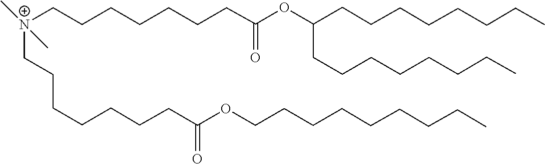 [SMILES: CCCCCCCCCOC(=O)CCCCCCC[N+](C)(C)CCCCCCCC(=O)OC(CCCCCCCC)CCCCCCCC] |
| I 339 | 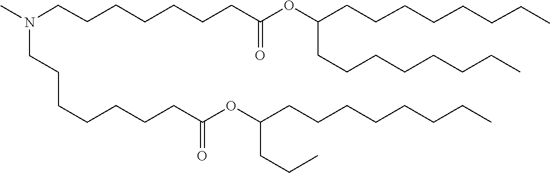 [SMILES: CCCCCCCCC(CCC)OC(=O)CCCCCCCN(C)CCCCCCCC(=O)OC(CCCCCCCC)CCCCCCCC] |
| I 340 | 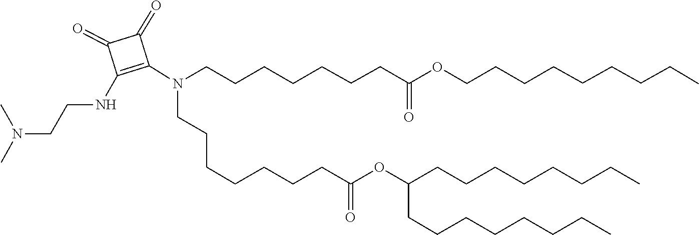 [SMILES: CCCCCCCCCOC(=O)CCCCCCCN(CCCCCCCC(=O)OC(CCCCCCCC)CCCCCCCC)c1c(NCCN(C)C)c(=O)c1=O] |
| I 341 | 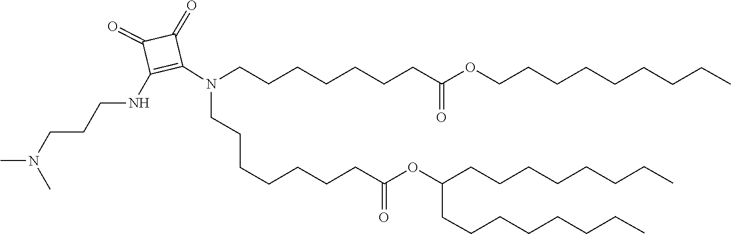 [SMILES: CCCCCCCCCOC(=O)CCCCCCCN(CCCCCCCC(=O)OC(CCCCCCCC)CCCCCCCC)c1c(NCCCN(C)C)c(=O)c1=O] |
| I 342 | 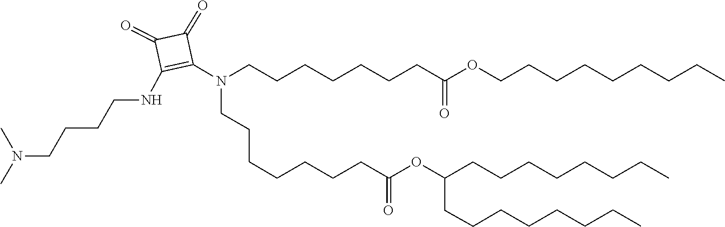 [SMILES: CCCCCCCCCOC(=O)CCCCCCCN(CCCCCCCC(=O)OC(CCCCCCCC)CCCCCCCC)c1c(NCCCCN(C)C)c(=O)c1=O] |
| I 343 | 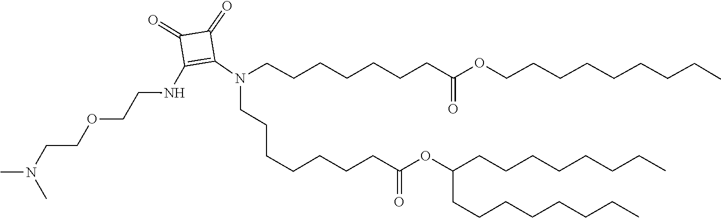 [SMILES: CCCCCCCCCOC(=O)CCCCCCCN(CCCCCCCC(=O)OC(CCCCCCCC)CCCCCCCC)c1c(NCCOCCN(C)C)c(=O)c1=O] |
| I 344 | 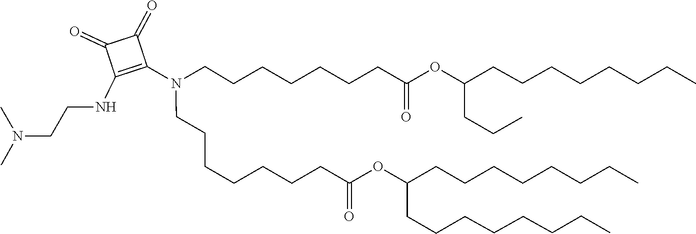 [SMILES: CCCCCCCCC(CCC)OC(=O)CCCCCCCN(CCCCCCCC(=O)OC(CCCCCCCC)CCCCCCCC)c1c(NCCN(C)C)c(=O)c1=O] |
| I 345 | 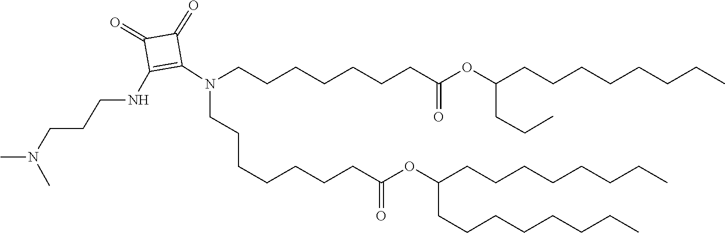 [SMILES: CCCCCCCCC(CCC)OC(=O)CCCCCCCN(CCCCCCCC(=O)OC(CCCCCCCC)CCCCCCCC)c1c(NCCCN(C)C)c(=O)c1=O] |
| I 346 | 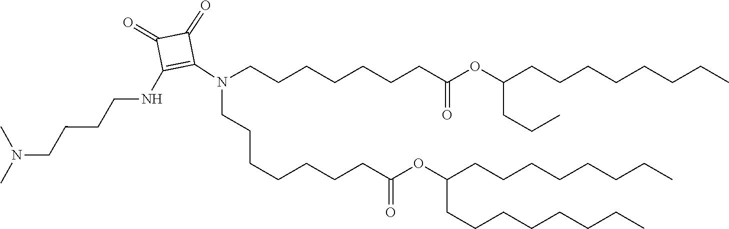 [SMILES: CCCCCCCCC(CCC)OC(=O)CCCCCCCN(CCCCCCCC(=O)OC(CCCCCCCC)CCCCCCCC)c1c(NCCCCN(C)C)c(=O)c1=O] |
| I 347 | 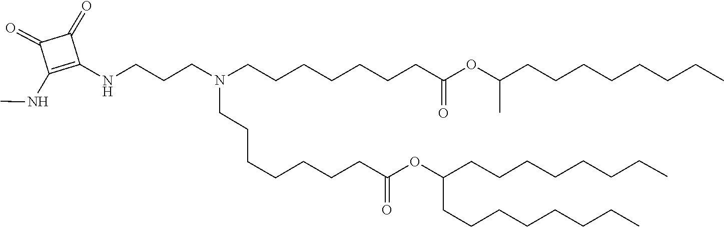 [SMILES: CCCCCCCCC(C)OC(=O)CCCCCCCN(CCCCCCCC(=O)OC(CCCCCCCC)CCCCCCCC)CCCNc1c(NC)c(=O)c1=O] |
| I 348 | 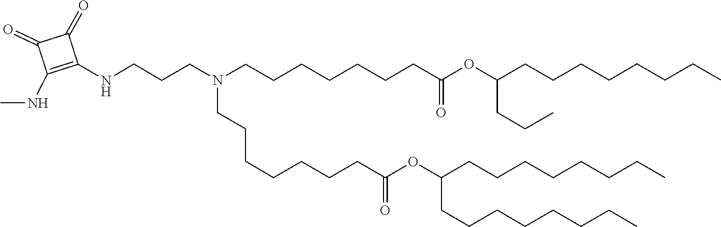 [SMILES: CCCCCCCCC(CCC)OC(=O)CCCCCCCN(CCCCCCCC(=O)OC(CCCCCCCC)CCCCCCCC)CCCNc1c(NC)c(=O)c1=O] |
| I 349 | 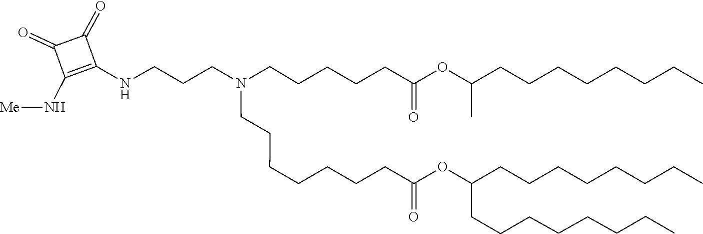 [SMILES: CCCCCCCCC(C)OC(=O)CCCCCN(CCCCCCCC(=O)OC(CCCCCCCC)CCCCCCCC)CCCNc1c(NC)c(=O)c1=O] |
| I 350 | 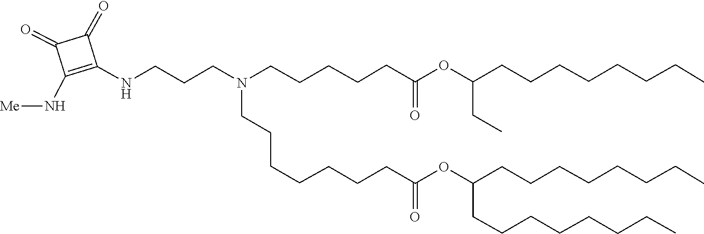 [SMILES: CCCCCCCCC(CC)OC(=O)CCCCCN(CCCCCCCC(=O)OC(CCCCCCCC)CCCCCCCC)CCCNc1c(NC)c(=O)c1=O] |
| I 351 | 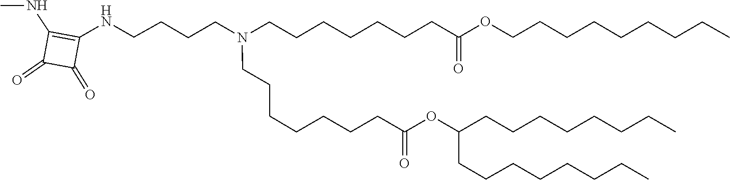 [SMILES: CCCCCCCCCOC(=O)CCCCCCCN(CCCCCCCC(=O)OC(CCCCCCCC)CCCCCCCC)CCCCNc1c(NC)c(=O)c1=O] |
| I 352 | 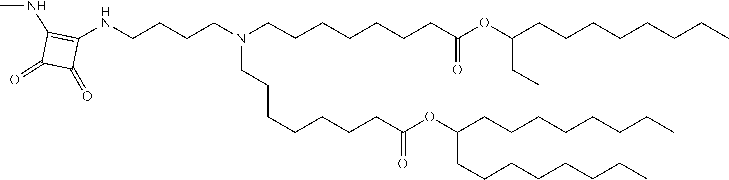 [SMILES: CCCCCCCCC(CC)OC(=O)CCCCCCCN(CCCCCCCC(=O)OC(CCCCCCCC)CCCCCCCC)CCCCNc1c(NC)c(=O)c1=O] |
| I 353 | 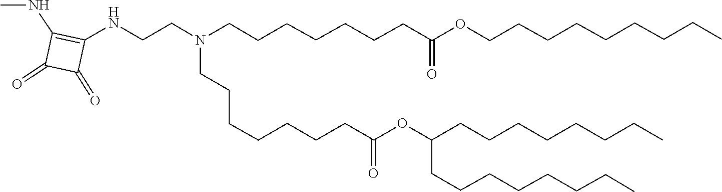 [SMILES: CCCCCCCCCOC(=O)CCCCCCCN(CCCCCCCC(=O)OC(CCCCCCCC)CCCCCCCC)CCNc1c(NC)c(=O)c1=O] |
| I 354 | 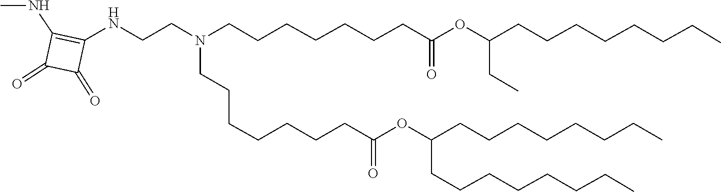 [SMILES: CCCCCCCCC(CC)OC(=O)CCCCCCCN(CCCCCCCC(=O)OC(CCCCCCCC)CCCCCCCC)CCNc1c(NC)c(=O)c1=O] |
| I 355 | 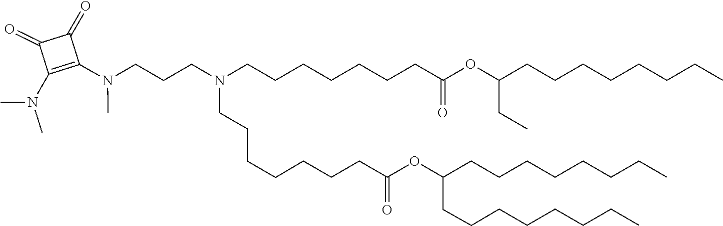 [SMILES: CCCCCCCCC(CC)OC(=O)CCCCCCCN(CCCCCCCC(=O)OC(CCCCCCCC)CCCCCCCC)CCCN(C)c1c(N(C)C)c(=O)c1=O] |

In some embodiments, a lipid of the disclosure comprises Compound I-340A:

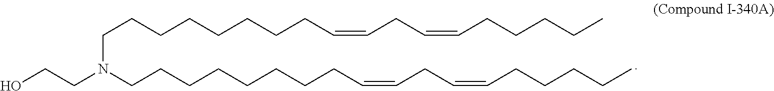 [SMILES: CCCCC/C=C\C/C=C\CCCCCCCCN(CCO)CCCCCCCC/C=C\C/C=C\CCCCC]

The central amine moiety of a lipid according to Formula (I I), (I IA), I (IB), I (II), (I IIa), (I III)), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), or (I VIIId) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some aspects, the ionizable lipids of the present disclosure may be one or more of compounds of formula I (I IX),

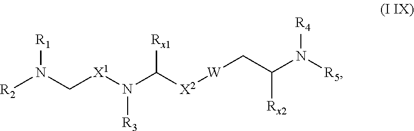

or salts or isomers thereof, wherein

W is

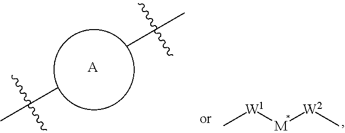

ring A is

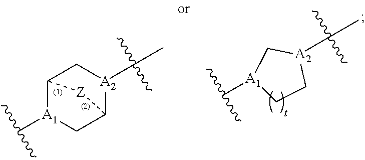

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

Rₓ₁ and Rₓ₂ are each independently H or C₁₋₃ alkyl;

each M is independently selected from the group consisting

of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

wherein when ring A is then

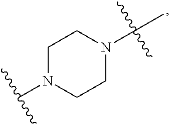 [SMILES: *N1CCN(*)CC1]

i) at least one of X¹, X², and X³ is not —CH₂—; and/or

ii) at least one of R₁, R₂, R₃, R₄, and R₅ is —R″MR′.

In some embodiments, the compound is of any of formulae (I IXa1)-(I IXa8):

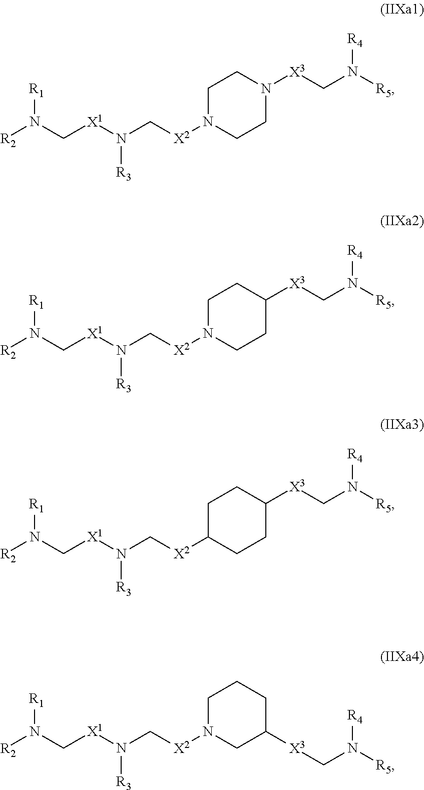

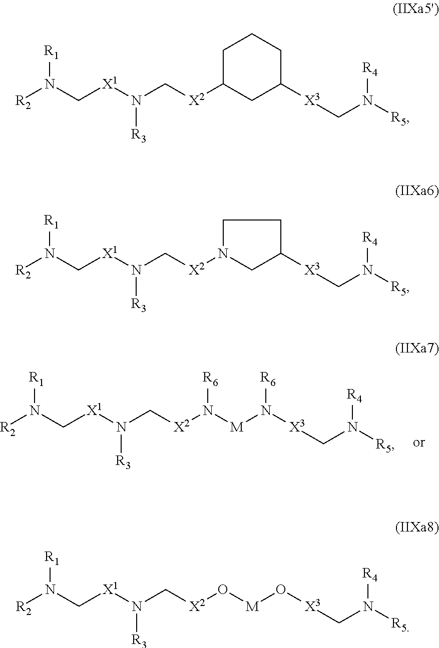

In some embodiments, the ionizable lipids are one or more of the compounds described in U.S. Application Nos. 62/271,146, 62/338,474, 62/413,345, and 62/519,826, and PCT Application No. PCT/US2016/068300.

In some embodiments, the ionizable lipids are selected from Compounds 1-156 described in U.S. Application No. 62/519,826.

In some embodiments, the ionizable lipids are selected from Compounds 1-16, 42-66, 68-76, and 78-156 described in U.S. Application No. 62/519,826.

In some embodiments, the ionizable lipid is

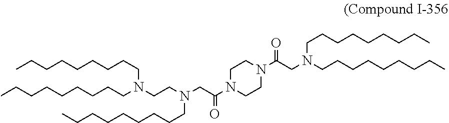 [SMILES: CCCCCCCCCN(CCCCCCCCC)CCN(CCCCCCCCC)CC(=O)N1CCN(C(=O)CN(CCCCCCCCC)CCCCCCCCC)CC1]

(also referred to herein as Compound M), or a salt thereof.

In some embodiments, the ionizable lipid is

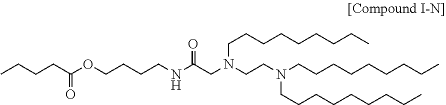 [SMILES: CCCCCCCCCN(CCCCCCCCC)CCN(CCCCCCCCC)CC(=O)NCCCCOC(=O)CCCC]

or a salt thereof.

In some embodiments, the ionizable lipid is

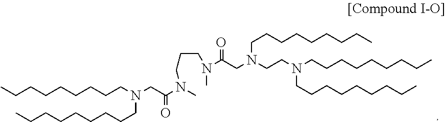 [SMILES: CCCCCCCCCN(CCCCCCCCC)CCN(CCCCCCCCC)CC(=O)N(C)CCCN(C)C(=O)CN(CCCCCCCCC)CCCCCCCCC]

or a salt thereof.

In some embodiments, the ionizable lipid is

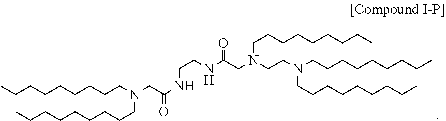 [SMILES: CCCCCCCCCN(CCCCCCCCC)CCN(CCCCCCCCC)CC(=O)NCCNC(=O)CN(CCCCCCCCC)CCCCCCCCC]

or a salt thereof.

In some embodiments, the ionizable lipid is

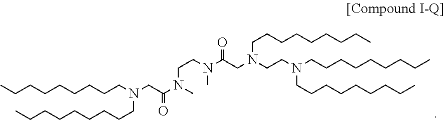 [SMILES: CCCCCCCCCN(CCCCCCCCC)CCN(CCCCCCCCC)CC(=O)N(C)CCN(C)C(=O)CN(CCCCCCCCC)CCCCCCCCC]

or a salt thereof.

The central amine moiety of a lipid according to any of the Formulae herein, e.g. a compound having any of Formula (I I), (I IA), (I IB), (II), (IIa), (Ib), (IIc), (IId), (Ile), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some embodiments, the amount the ionizable amino lipid of the disclosure, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8)) (each of these preceded by the letter I for clarity) ranges from about 1 mol % to 99 mol % in the lipid composition.

In one embodiment, the amount of the ionizable amino lipid of the disclosure, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the ionizable amino lipid of the disclosure, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the ionizable amino lipid of the disclosure, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 45 mol % in the lipid composition.

In one specific embodiment, the amount of the ionizable amino lipid of the disclosure, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 40 mol % in the lipid composition.

In one specific embodiment, the amount of the ionizable amino lipid of the disclosure, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 50 mol % in the lipid composition.

In addition to the ionizable amino lipid disclosed herein, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8), (each of these preceded by the letter I for clarity) the lipid-based composition (e.g., lipid nanoparticle) disclosed herein can comprise additional components such as cholesterol and/or cholesterol analogs, non-cationic helper lipids, structural lipids, PEG-lipids, and any combination thereof.

Additional ionizable lipids of the disclosure can be selected from the non-limiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), (13Z,165Z)—N,N-dimethyl-3-nonydocosa-13-16-dien-1-amine (L608), 2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-die n-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids of the disclosure can also be the compounds disclosed in International Publication No. WO 2017/075531 A1, hereby incorporated by reference in its entirety. For example, the ionizable amino lipids include, but not limited to:

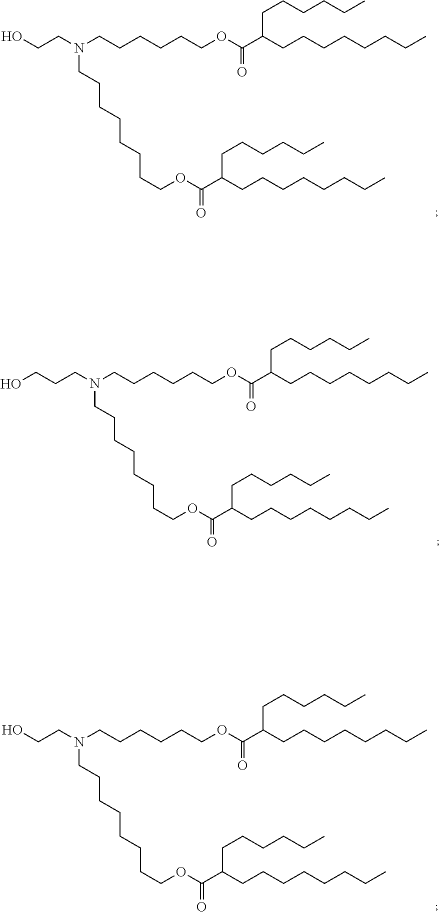 [SMILES: CCCCCCCCC(CCCCCC)C(=O)OCCCCCCCCN(CCO)CCCCCCOC(=O)C(CCCCCC)CCCCCCCC ; CCCCCCCCC(CCCCCC)C(=O)OCCCCCCCCN(CCCO)CCCCCCOC(=O)C(CCCCCC)CCCCCCCC ; CCCCCCCCC(CCCCCC)C(=O)OCCCCCCCCN(CCO)CCCCCCOC(=O)C(CCCCCC)CCCCCCCC]

and any combination thereof.

Ionizable lipids of the disclosure can also be the compounds disclosed in International Publication No. WO 2015/199952 A1, hereby incorporated by reference in its entirety. For example, the ionizable amino lipids include, but not limited to:

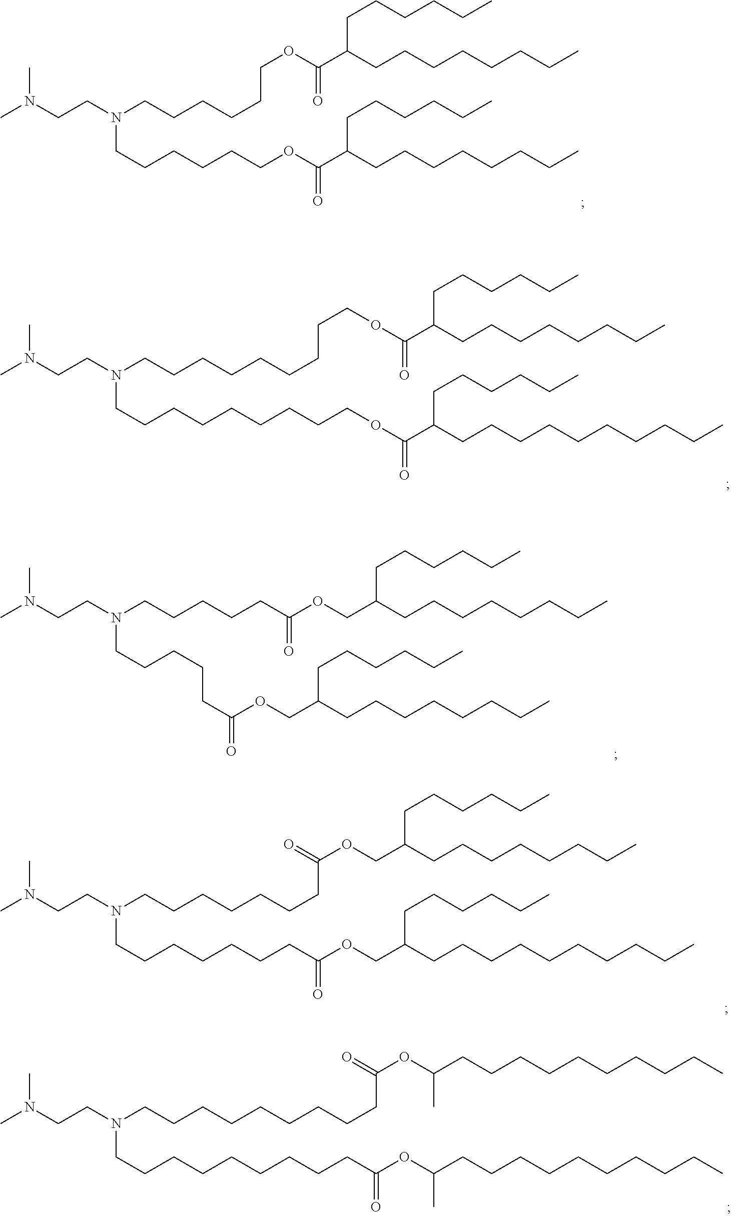

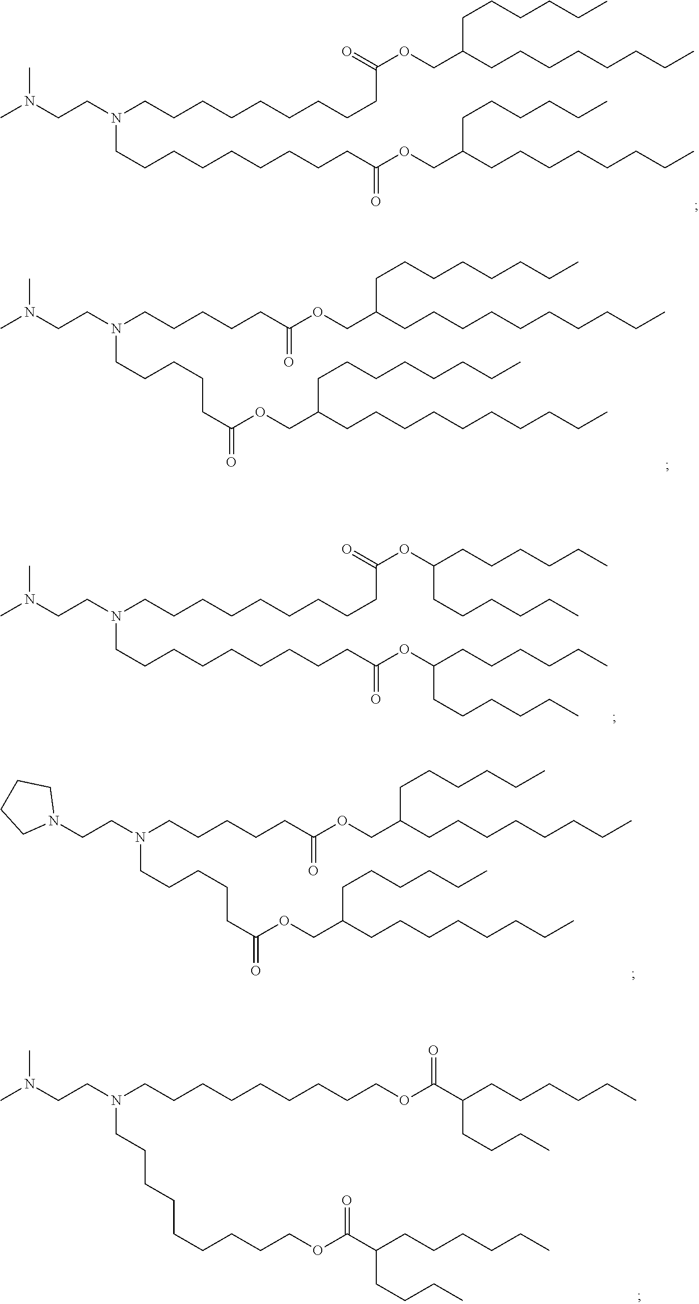

and any combination thereof.

In any of the foregoing or related aspects, the ionizable lipid of the LNP of the disclosure comprises a compound included in any e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity).

In any of the foregoing or related aspects, the ionizable lipid of the LNP of the disclosure comprises a compound comprising any of Compound Nos. I 1-356.

In any of the foregoing or related aspects, the ionizable lipid of the LNP of the disclosure comprises at least one compound selected from the group consisting of: Compound Nos. I 18 (also referred to as Compound X), I 25 (also referred to as Compound Y), I 48, I 50, I 109, I 111, I 113, I 181, I 182, I 244, I 292, I 301, I 321, I 322, I 326, I 328, I 330, I 331, and I 332. In another embodiment, the ionizable lipid of the LNP of the disclosure comprises a compound selected from the group consisting of: Compound Nos. I 18 (also referred to as Compound X), I 25 (also referred to as Compound Y), I 48, I 50, I 109, I 111, I 181, I 182, I 292, I 301, I 321, I 326, I 328, and I 330. In another embodiment, the ionizable lipid of the LNP of the disclosure comprises a compound selected from the group consisting of: Compound Nos. I 182, I 301, I 321, and I 326.

In any of the foregoing or related aspects, the synthesis of compounds of the disclosure, e.g. compounds comprising any of Compound Nos. 1-356, follows the synthetic descriptions in U.S. Provisional Patent Application No. 62/733,315, filed Sep. 19, 2018.

### Representative Synthetic Routes:

### Compound I-182: Heptadecan-9-yl 8-((3-((2-(methylamino)-3,4-dioxocyclobut-1-en-1-yl)amino)propyl)(8-(nonyloxy)-8-oxooctyl)amino)octanoate 3-Methoxy-4-(methylamino)cyclobut-3-ene-1,2-dione

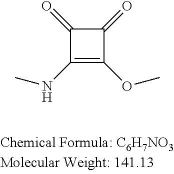

To a solution of 3,4-dimethoxy-3-cyclobutene-1,2-dione (1 g, 7 mmol) in 100 mL diethyl ether was added a 2M methylamine solution in THF (3.8 mL, 7.6 mmol) and a ppt. formed almost immediately. The mixture was stirred at rt for 24 hours, then filtered, the filter solids washed with diethyl ether and air-dried. The filter solids were dissolved in hot EtOAc, filtered, the filtrate allowed to cool to room temp., then cooled to 0° C. to give a ppt. This was isolated via filtration, washed with cold EtOAc, air-dried, then dried under vacuum to give 3-methoxy-4-(methylamino)cyclobut-3-ene-1,2-dione (0.70 g, 5 mmol, 73%) as a white solid. ¹H NMR (300 MHz, DMSO-d₆) δ: ppm 8.50 (br. d, 1H, J=69 Hz); 4.27 (s, 3H); 3.02 (sdd, 3H, J=42 Hz, 4.5 Hz).

### Heptadecan-9-yl 8-((3-((2-(methylamino)-3,4-dioxocyclobut-1-en-1-yl)amino)propyl)(8-(nonyloxy)-8-oxooctyl)amino)octanoate

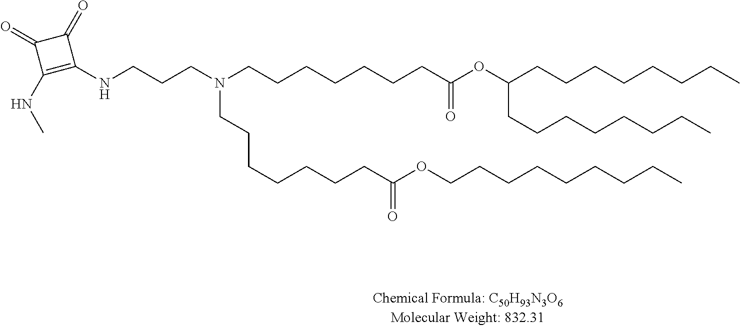 [SMILES: CCCCCCCCCOC(=O)CCCCCCCN(CCCCCCCC(=O)OC(CCCCCCCC)CCCCCCCC)CCCNc1c(NC)c(=O)c1=O]

To a solution of heptadecan-9-yl 8-((3-aminopropyl)(8-(nonyloxy)-8-oxooctyl)amino)octanoate (200 mg, 0.28 mmol) in 10 mL ethanol was added 3-methoxy-4-(methylamino)cyclobut-3-ene-1,2-dione (39 mg, 0.28 mmol) and the resulting colorless solution stirred at rt for 20 hours after which no starting amine remained by LC/MS. The solution was concentrated in vacuo and the residue purified by silica gel chromatography (0-100% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-(nonyloxy)-8-oxooctyl)amino)octanoate (138 mg, 0.17 mmol, 60%) as a gummy white solid. UPLC/ELSD: RT=3. min. MS (ES): m/z (MH⁺) 833.4 for C₅₁H₉₅N₃O₆. ¹H NMR (300 MHz, CDCl₃) δ: ppm 7.86 (br. s., 1H); 4.86 (quint., 1H, J=6 Hz); 4.05 (t, 2H, J=6 Hz); 3.92 (d, 2H, J=3 Hz); 3.20 (s, 6H); 2.63 (br. s, 2H); 2.42 (br. s, 3H); 2.28 (m, 4H); 1.74 (br. s, 2H); 1.61 (m, 8H); 1.50 (m, 5H); 1.41 (m, 3H); 1.25 (br. m, 47H); 0.88 (t, 9H, J=7.5 Hz).

### Compound I-301: Heptadecan-9-yl 8-((3-((2-(methylamino)-3,4-dioxocyclobut-1-en-1-yl)amino)propyl)(8-oxo-8-(undecan-3-yloxy)octyl)amino)octanoate

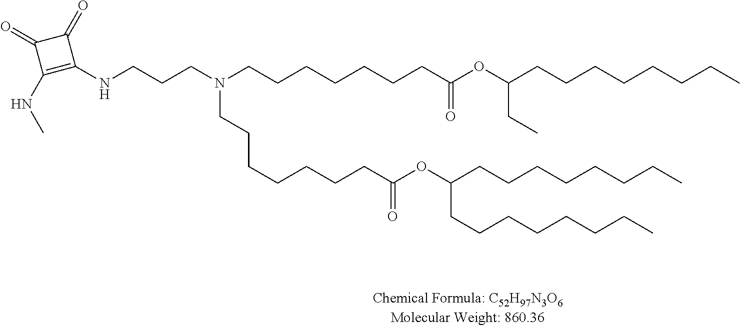 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

Compound I-301 was prepared analogously to compound 182 except that heptadecan-9-yl 8-((3-aminopropyl)(8-oxo-8-(undecan-3-yloxy)octyl)amino)octanoate (500 mg, 0.66 mmol) was used instead of heptadecan-9-yl 8-((3-aminopropyl)(8-(nonyloxy)-8-oxooctyl)amino)octanoate. Following an aqueous workup the residue was purified by silica gel chromatography (0-50% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-oxo-8-(undecan-3-yloxy)octyl)amino)octanoate (180 mg, 32%) as a white waxy solid. HPLC/UV (254 nm): RT=6.77 min. MS (CI): m/z (MH⁺) 860.7 for C₅₂H₉₇N₃O₆. ¹H NMR (300 MHz, CDCl₃): δ ppm 4.86-4.79 (m, 2H); 3.66 (bs, 2H); 3.25 (d, 3H, J=4.9 Hz); 2.56-2.52 (m, 2H); 2.42-2.37 (m, 4H); 2.28 (dd, 4H, J=2.7 Hz, 7.4 Hz); 1.78-1.68 (m, 3H); 1.64-1.50 (m, 16H); 1.48-1.38 (m, 6H); 1.32-1.18 (m, 43H); 0.88-0.84 (m, 12H).

(i) Cholesterol/Structural Lipids

The immune cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

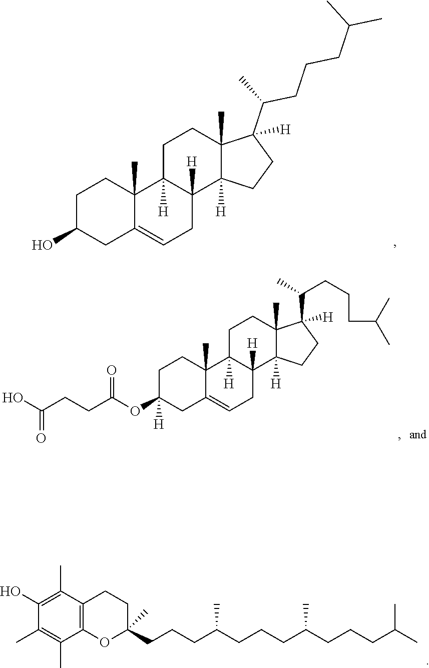

The immune cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. Structural lipids can include, but are not limited to, sterols (e.g., phytosterols or zoosterols).

In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds S1-148 in Tables 1-16 herein.

In certain embodiments, the structural lipid is cholesterol. In certain embodiments, the structural lipid is an analog of cholesterol.

In certain embodiments, the structural lipid is alpha-tocopherol.

In an aspect, the structural lipid of the disclosure features a compound having the structure of Formula SI:

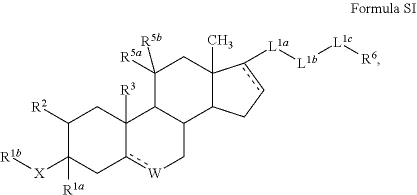

where

R^{1a} is H, optionally substituted C₁-C₆ alkyl, optionally substituted C₂-C₆ alkenyl, or optionally substituted C₂-C₆ alkynyl;

X is O or S; R^{1b} is H, optionally substituted C₁-C₆ alkyl, or

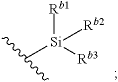

each of R^{b1}, R^{b2}, and R^{b3} is, independently, optionally substituted C₁-C₆ alkyl or optionally substituted C₆-C₁₀ aryl;

R² is H or OR^{A}, where R^{A} is H or optionally substituted C₁-C₆ alkyl;

R³ is H or

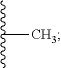

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

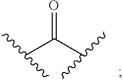 [SMILES: *C(*)=O]

L^{1a} is absent,

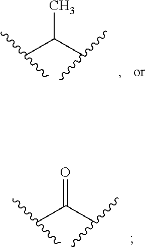

L^{1b} is absent,

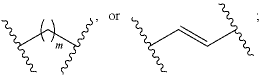

m is 1, 2, or 3;

L^{1c} is absent,

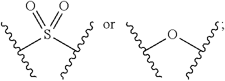

and

R⁶ is optionally substituted C₃-C₁₀ cycloalkyl, optionally substituted C₃-C₁₀ cycloalkenyl, optionally substituted C₆-C₁₀ aryl, optionally substituted C₂-C₉ heterocyclyl, or optionally substituted C₂-C₉ heteroaryl, or a pharmaceutically acceptable salt thereof.

In some embodiments, the compound has the structure of Formula SIa:

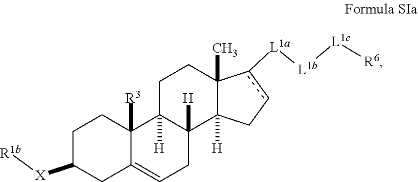

or a pharmaceutically acceptable salt thereof.

In some embodiments, the compound has the structure of Formula SIb:

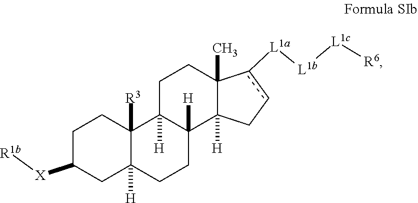

or a pharmaceutically acceptable salt thereof.

In some embodiments, the compound has the structure of Formula SIc:

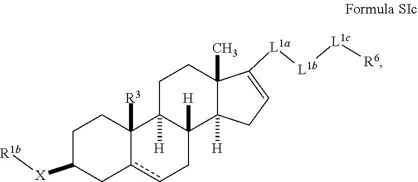

or a pharmaceutically acceptable salt thereof.

In some embodiments, the compound has the structure of Formula SId:

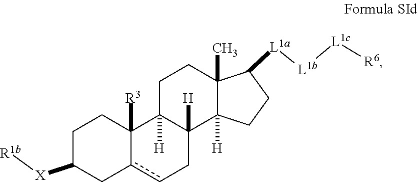

or a pharmaceutically acceptable salt thereof.

In some embodiments, L^{1a} is absent. In some embodiments, L^{1a} is

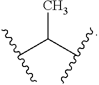 [SMILES: *C(*)C]

In some embodiments, L^{1a} is

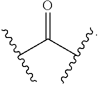 [SMILES: *C(*)=O]

In some embodiments, L^{1b} is absent. In some embodiments, L^{1b} is

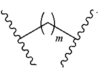

In some embodiments, L^{1b} is

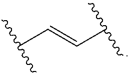 [SMILES: */C=C/*]

In some embodiments, m is 1 or 2. In some embodiments, m is 1. In some embodiments, m is 2.

In some embodiments, L^{1c} is absent. In some embodiments, L^{1c} is

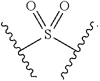 [SMILES: *S(*)(=O)=O]

In some embodiments, L^{1c} is

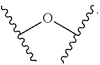 [SMILES: *O*]

In some embodiments, R⁶ is optionally substituted C₆-C₁₀ aryl.

In some embodiments, R⁶ is

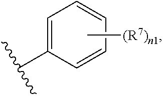

where

n1 is 0, 1, 2, 3, 4, or 5; and

each R⁷ is, independently, halo or optionally substituted C₁-C₆ alkyl.

In some embodiments, each R⁷ is, independently,

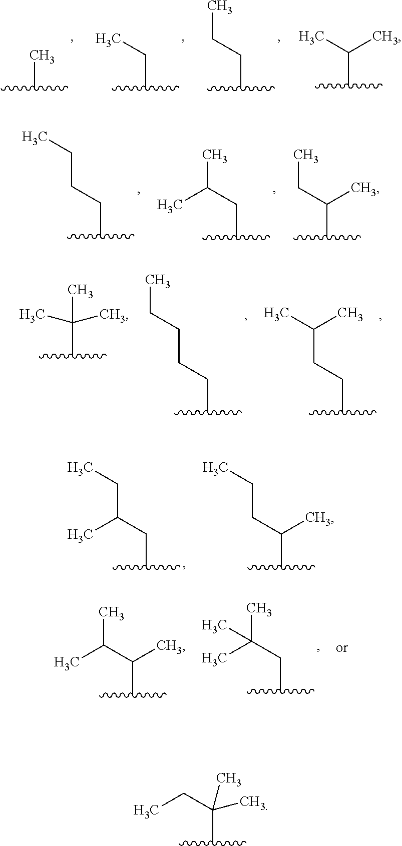

In some embodiments, n1 is 0, 1, or 2. In some embodiments, n is 0. In some embodiments, n1 is 1. In some embodiments, n1 is 2.

In some embodiments, R⁶ is optionally substituted C₃-C₁₀ cycloalkyl.

In some embodiments, R⁶ is optionally substituted C₃-C₁₀ monocycloalkyl.

In some embodiments, R⁶ is

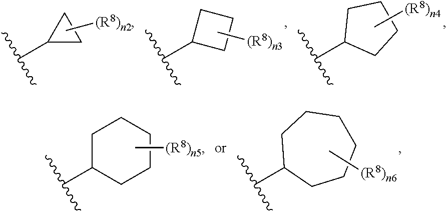

where

n2 is 0, 1, 2, 3, 4, or 5;
n3 is 0, 1, 2, 3, 4, 5, 6, or 7;
n4 is 0, 1, 2, 3, 4, 5, 6, 7, 8, or 9;
n5 is 0, 1, 2, 3, 4, 5, 6, 7, 8, 9, 10, or 11;
n6 is 0, 1, 2, 3, 4, 5, 6, 7, 8, 9, 10, 11, 12, or 13; and

each R⁸ is, independently, halo or optionally substituted C₁-C₆ alkyl.

In some embodiments, each R⁸ is, independently.

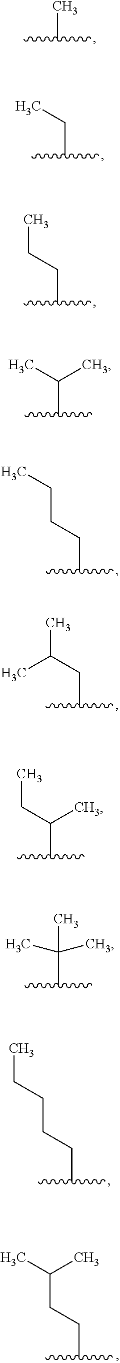

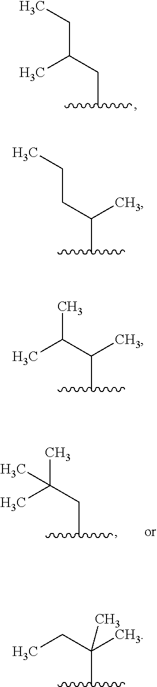

In some embodiments, R⁶ is optionally substituted C₃-C₁₀ polycycloalkyl.

In some embodiments, R⁶ is

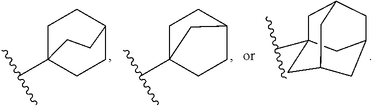

In some embodiments, R⁶ is optionally substituted C₃-C₁₀ cycloalkenyl.

In some embodiments, R⁶ is

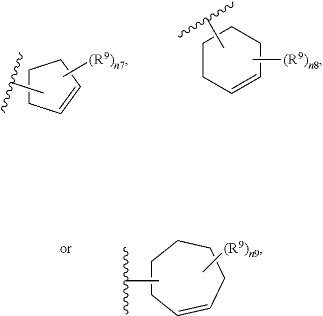

where

n7 is 0, 1, 2, 3, 4, 5, 6, or 7;

n8 is 0, 1, 2, 3, 4, 5, 6, 7, 8, or 9;

n9 is 0, 1, 2, 3, 4, 5, 6, 7, 8, 9, 10, or 11; and

each R⁹ is, independently, halo or optionally substituted C₁-C₆ alkyl.

In some embodiments, R⁶ is

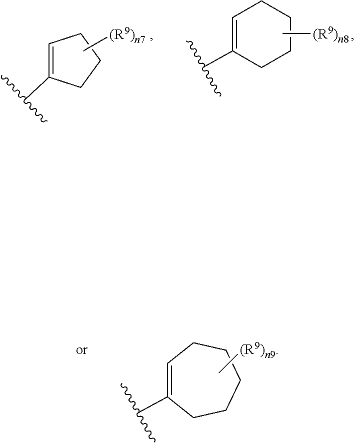

In some embodiments, each R⁹ is, independently,

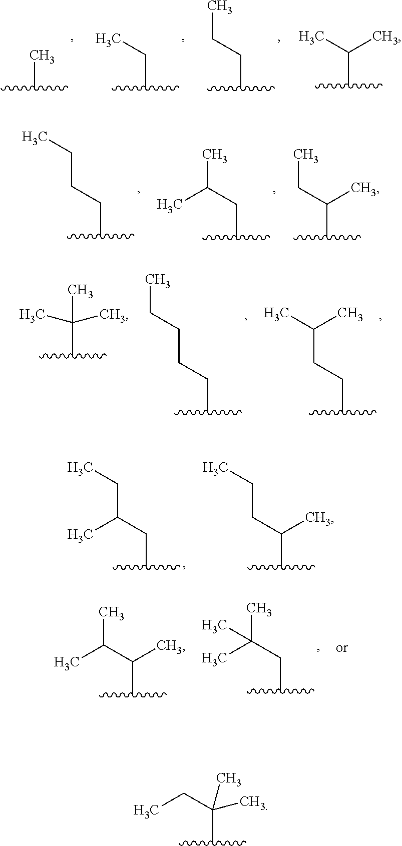

In some embodiments, R⁶ is optionally substituted C₂-C₉ heterocyclyl.

In some embodiments, R⁶ is

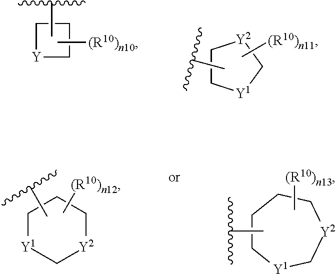

where

n10 is 0, 1, 2, 3, 4, or 5;
n11 is 0, 1, 2, 3, 4, or 5;
n12 is 0, 1, 2, 3, 4, 5, 6, or 7;
n13 is 0, 1, 2, 3, 4, 5, 6, 7, 8, or 9;
each R¹⁰ is, independently, halo or optionally substituted C₁-C₆ alkyl; and

each of Y¹ and Y² is, independently, O, S, NR^{B}, or CR^{11a}R^{11b},

where R^{B} is H or optionally substituted C₁-C₆ alkyl;
each of R^{11a} and R^{11b} is, independently, H, halo, or optionally substituted C₁-C₆ alkyl; and

if Y² is CR^{11a}R^{11b}, then Y¹ is O, S, or NR^{B}.

In some embodiments, Y¹ is O.

In some embodiments, Y² is O. In some embodiments, Y² is CR^{11a}R^{11b}.

In some embodiments, each R¹⁰ is, independently,

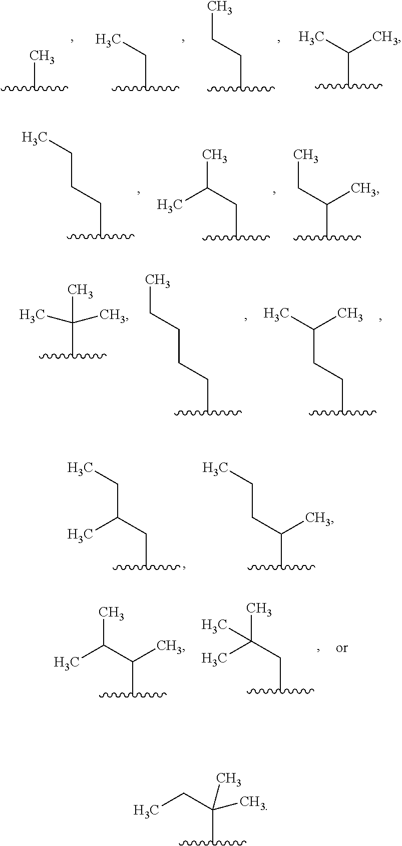

In some embodiments, R⁶ is optionally substituted C₂-C₉ heteroaryl.

In some embodiments, R⁶ is

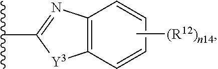

where

Y³ is NR^{C}, O, or S

n14 is 0, 1, 2, 3, or 4;

R^{C} is H or optionally substituted C₁-C₆ alkyl; and

each R¹² is, independently, halo or optionally substituted C₁-C₆ alkyl.

In some embodiments, R⁶ is

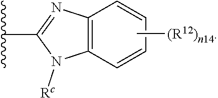

In some embodiments, R⁶ is

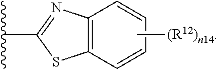

In an aspect, the structural lipid of the disclosure features a compound having the structure of Formula SII:

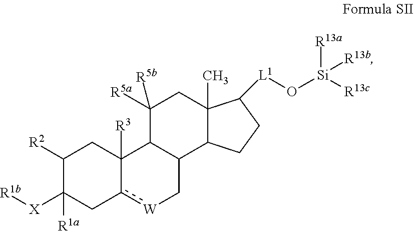

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

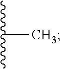

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

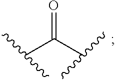 [SMILES: *C(*)=O]

L¹ is optionally substituted C₁-C₆ alkylene; and

each of R^{13a}, R^{13b}, and R^{13c} is, independently, optionally substituted C₁-C₆ alkyl or optionally substituted C₆-C₁₀ aryl,

or a pharmaceutically acceptable salt thereof.

In some embodiments, the compound has the structure of Formula SIIa:

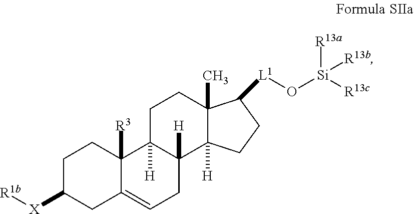

or a pharmaceutically acceptable salt thereof.

In some embodiments, the compound has the structure of Formula SIIb:

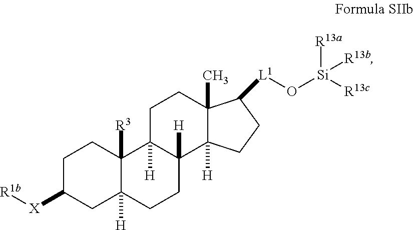

or a pharmaceutically acceptable salt thereof.

In some embodiments, L¹ is

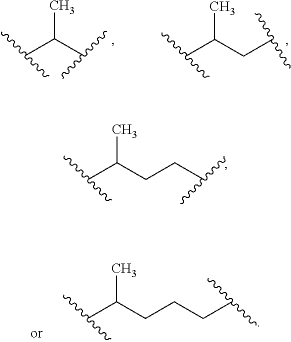

In some embodiments, each of R^{13a}, R^{13b}, and R^{13c} is, independently,

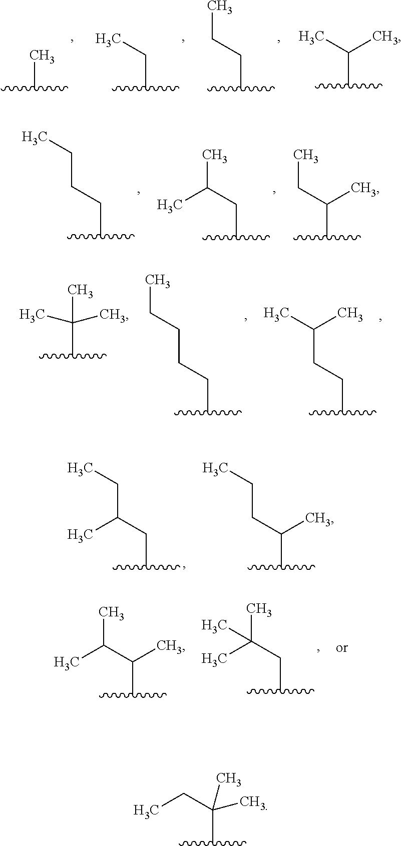

In an aspect, the structural lipid of the disclosure features a compound having the structure of Formula SIII:

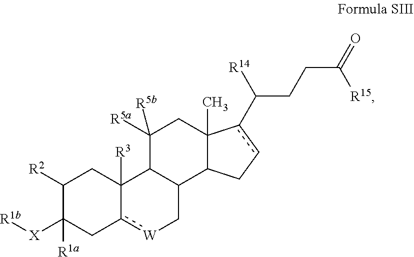

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

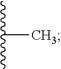

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, hydroxyl, optionally substituted C₁-C₆ alkyl, —OS(O)₂R^{4c}, where R^{4c} is optionally substituted C₁-C₆ alkyl or optionally substituted C₆-C₁₀ aryl;

each of R^{5a} and R^{5b} is, independently, H or OR^{A}, or R^{5a} and R^{5b}, together with the atom to which each is attached, combine to form

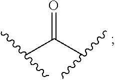 [SMILES: *C(*)=O]

R¹⁴ is H or C₁-C₆ alkyl; and R¹⁵ is

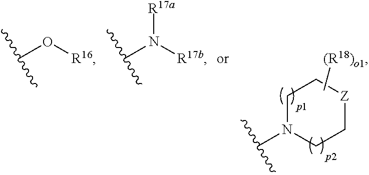

where

R¹⁶ is H or optionally substituted C₁-C₆ alkyl;
R^{17b} is H, OR^{17c}, optionally substituted C₆-C₁₀ aryl, or optionally substituted C₁-C₆ alkyl;

R^{17c} is H or optionally substituted C₁-C₆ alkyl;

o1 is 0, 1, 2, 3, 4, 5, 6, 7, or 8;

p1 is 0, 1, or 2;

p2 is 0, 1, or 2;

Z is CH₂ O, S, or NR^{D}, where R^{D} is H or optionally substituted C₁-C₆ alkyl; and

each R¹⁸ is, independently, halo or optionally substituted C₁-C₆ alkyl, or a pharmaceutically acceptable salt thereof.

In some embodiments, the compound has the structure of Formula SIIIa:

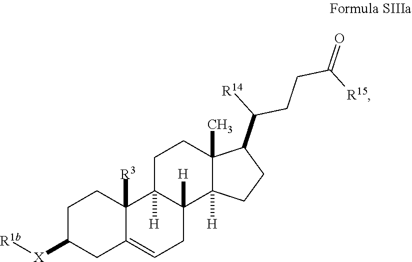

or a pharmaceutically acceptable salt thereof.

In some embodiments, the compound has the structure of Formula SIIIb:

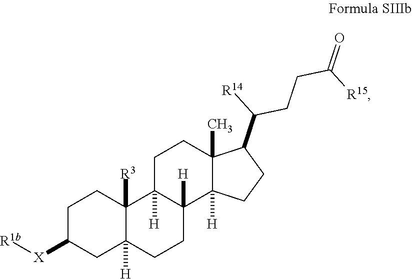

or a pharmaceutically acceptable salt thereof.

In some embodiments, R¹⁴ is H,

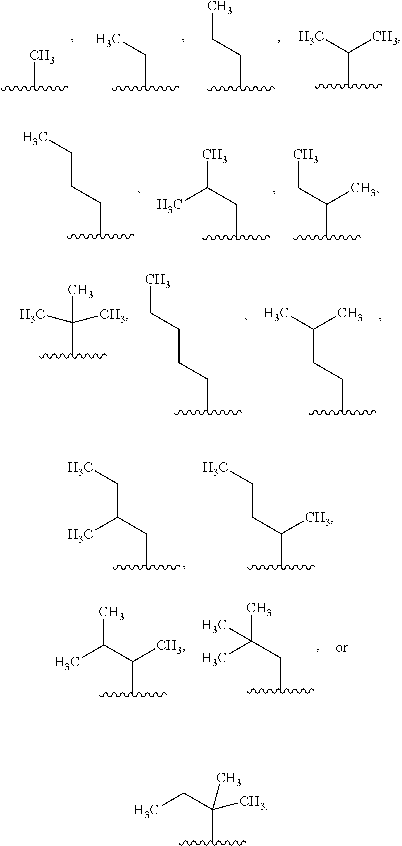

In some embodiments, R¹⁴ is

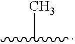

In some embodiments, R¹⁵ is

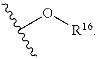

In some embodiments, R¹⁵ is

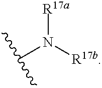

In some embodiments, R¹⁶ is H. In some embodiments, R¹⁶ is

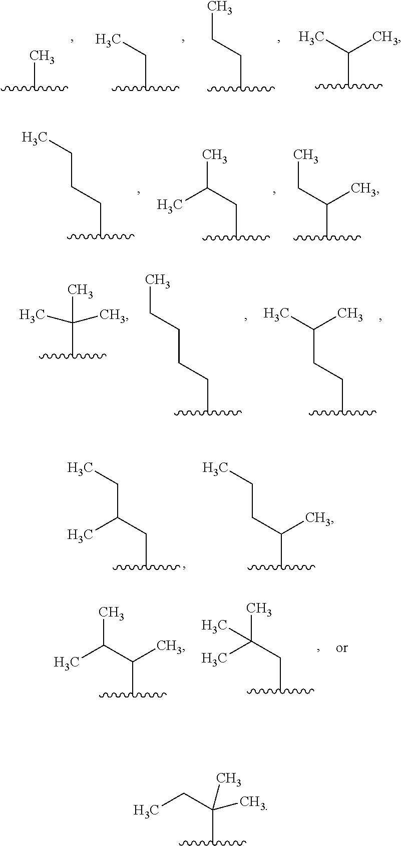

In some embodiments, R^{17a} is H. In some embodiments, R^{17a} is optionally substituted C₁-C₆ alkyl.

In some embodiments, R^{17b} is H. In some embodiments, R^{17b} optionally substituted C₁-C₆ alkyl. In some embodiments, R^{17b} is OR^{17c}.

In some embodiments, R^{17c} is H,

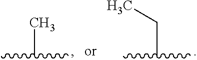

In some embodiments, R^{17c} is H. In some embodiments, R^{17c} is

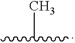

In some embodiments, R¹⁵ is

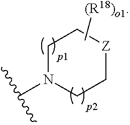

In some embodiments, each R¹⁸ is, independently,

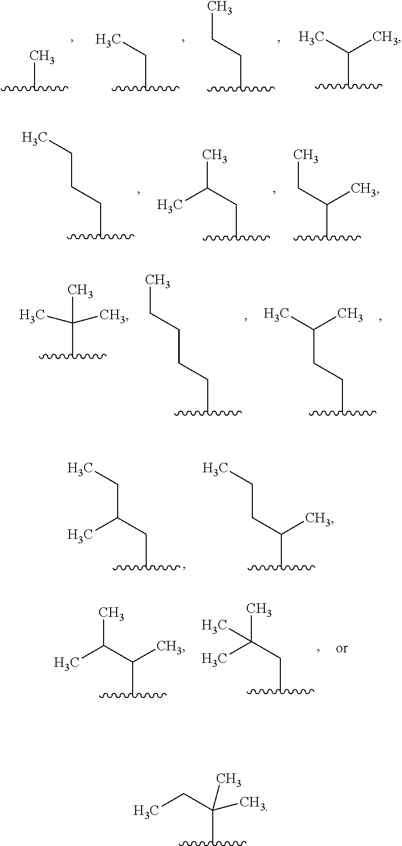

In some embodiments, Z is CH₂. In some embodiments, Z is O. In some embodiments, Z is NR^{D}.

In some embodiments, o1 is 0, 1, 2, 3, 4, 5, or 6.

In some embodiments, o1 is 0. In some embodiments, o1 is 1. In some embodiments, o1 is 2. In some embodiments, o1 is 3. In some embodiments, o1 is 4. In some embodiments, o1 is 5. In some embodiments, o1 is 6.

In some embodiments, p1 is 0 or 1. In some embodiments, p1 is 0. In some embodiments, p1 is 1.

In some embodiments, p2 is 0 or 1. In some embodiments, p2 is 0. In some embodiments, p2 is 1.

In an aspect, the structural lipid of the disclosure features a compound having the structure of Formula SIV:

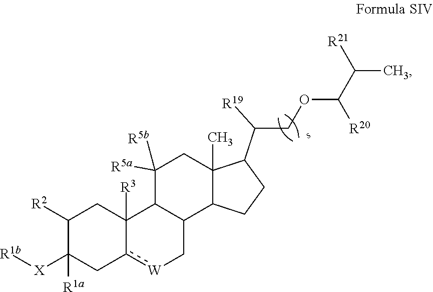

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

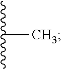

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

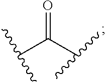 [SMILES: *C(*)=O]

s is 0 or 1;

R¹⁹ is H or C₁-C₆ alkyl;

R²⁰ is C₁-C₆ alkyl;

R²¹ is H or C₁-C₆ alkyl,

or a pharmaceutically acceptable salt thereof.

In some embodiments, the compound has the structure of Formula SIVa:

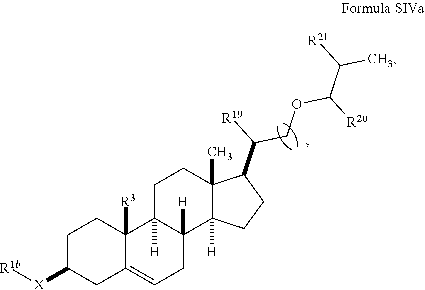

or a pharmaceutically acceptable salt thereof.

In some embodiments, the compound has the structure of Formula SIVb:

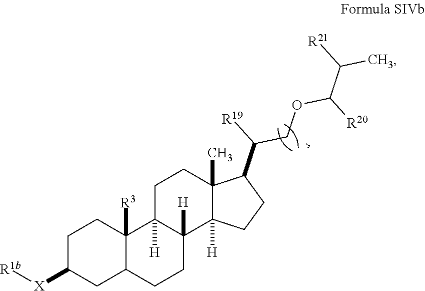

or a pharmaceutically acceptable salt thereof.

In some embodiments, R¹⁹ is H,

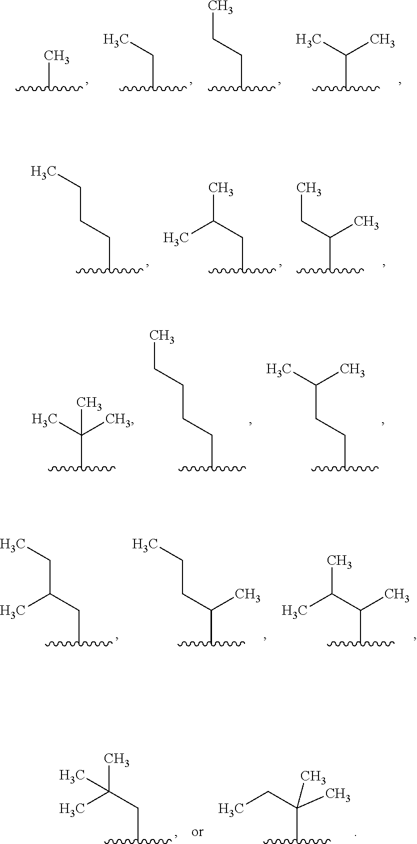

In some embodiments, R¹⁹ is

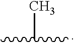

In some embodiments, R²⁰ is,

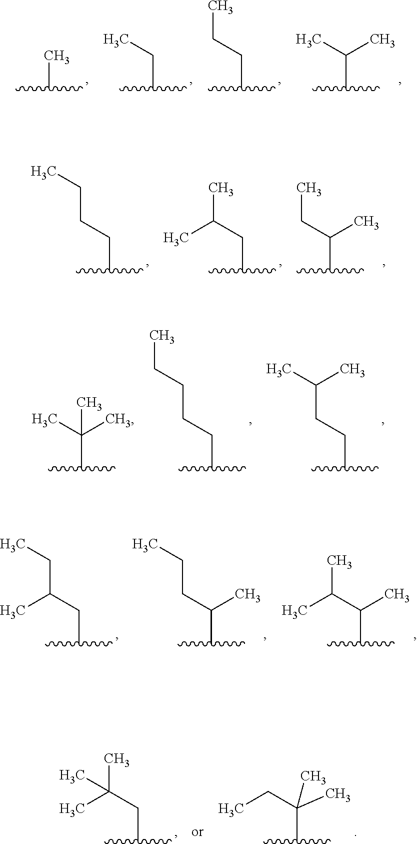

In some embodiments, R²¹ is H,

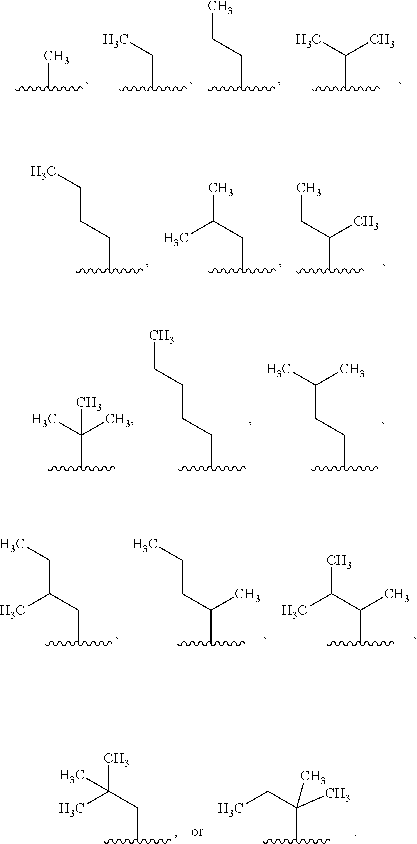

In an aspect, the structural lipid of the disclosure features, a compound having the structure of Formula SV:

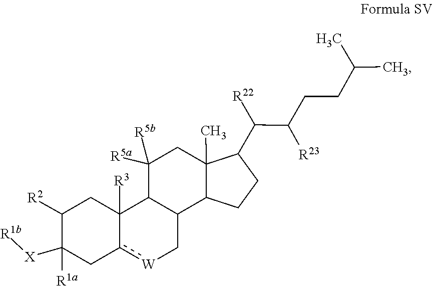

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

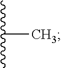

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to hich each is attached, combine to form

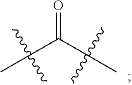 [SMILES: *C(*)=O]

R²² is H or C₁-C₆ alkyl; and

R²³ is halo, hydroxyl, optionally substituted C₁-C₆ alkyl, or optionally substituted C₁-C₆ heteroalkyl,

or a pharmaceutically acceptable salt thereof.

In some embodiments, the compound has the structure of Formula SVa:

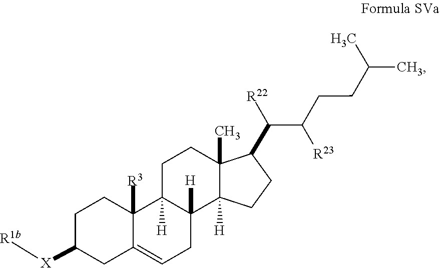

or a pharmaceutically acceptable salt thereof.

In some embodiments, the compound has the structure of Formula SVb:

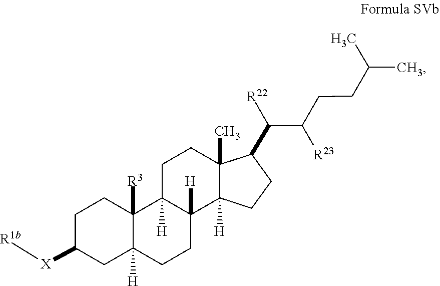

or a pharmaceutically acceptable salt thereof.

In some embodiments, R²² is H,

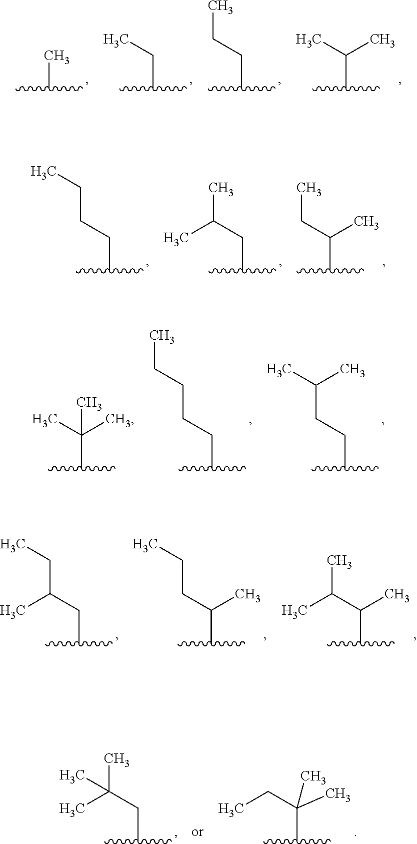

In some embodiments, R²² is

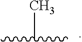

In some embodiments, R²³ is

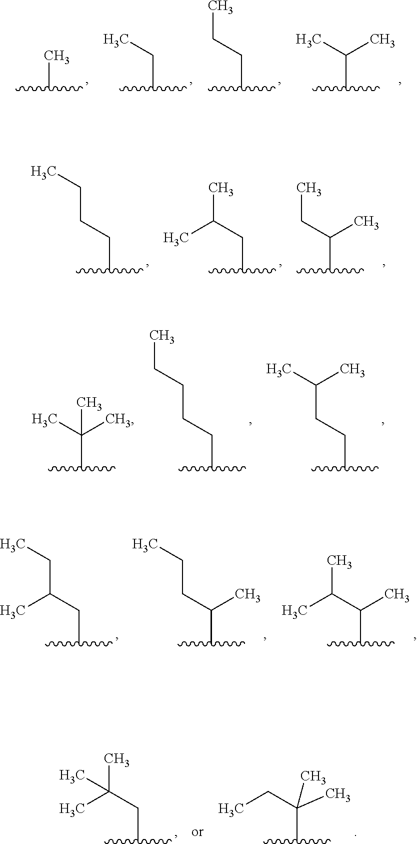

In an aspect, the structural lipid of the disclosure features a compound having the structure of Formula SVI:

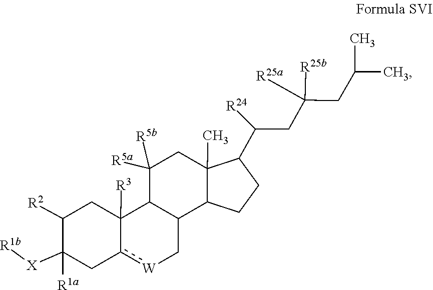

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

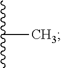

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

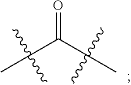 [SMILES: *C(*)=O]

R²⁴ is H or C₁-C₆ alkyl; and

each of R^{25a} and R^{25b} is C₁-C₆ alkyl,

or a pharmaceutically acceptable salt thereof.

In some embodiments, the compound has the structure of Formula SVIa:

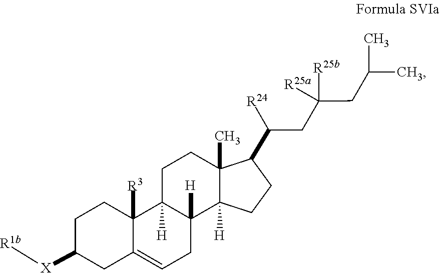

or a pharmaceutically acceptable salt thereof.

In some embodiments, the compound has the structure of Formula SVIb:

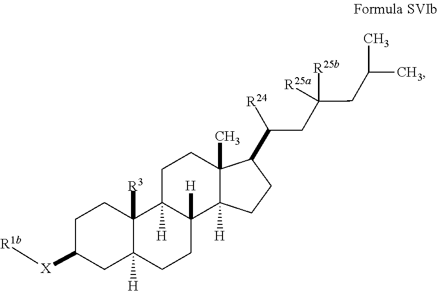

or a pharmaceutically acceptable salt thereof.

In some embodiments, R²⁴ is H,

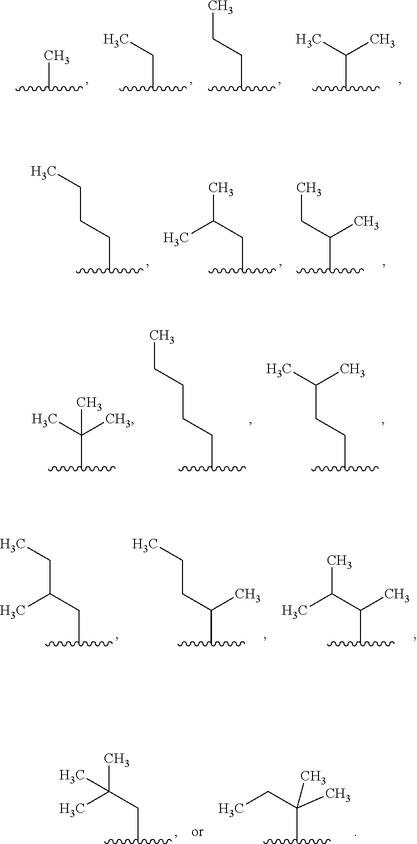

In some embodiments, R²⁴ is

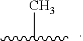

In some embodiments, each of R^{25a} and R^{25b} is, independently,

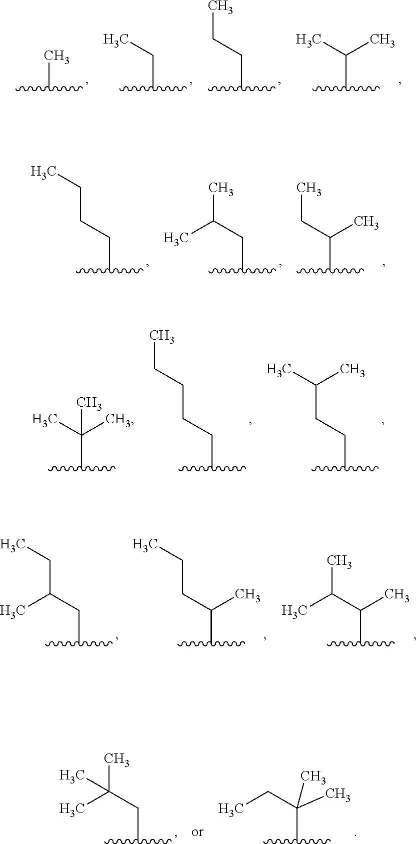

In an aspect, the structural lipid of the disclosure features a compound having the structure of Formula SVII:

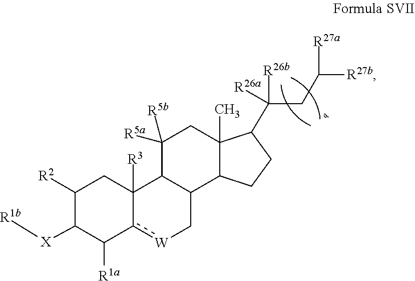

where

R^{1a} is H, optionally substituted C₁-C₆ alkyl, optionally substituted C₂-C₆ alkenyl, optionally substituted C₂-C₆ alkynyl, or

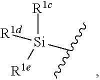

where each of R^{1c}, R^{1d}, and R^{1e} is, independently, optionally substituted C₁-C₆ alkyl or optionally substituted C₆-C₁₀ aryl;

X is O or S;

R^{1b} is H or optionally substituted C₁-C₆ alkyl;

R² is H or OR^{A}, where R^{A} is H or optionally substituted C₁-C₆ alkyl;

R³ is H or

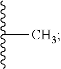

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to

which each is attached, combine to form

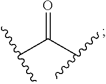 [SMILES: *C(*)=O]

q is 0 or 1;

each of R^{26a} and R^{26b} is, independently, H or optionally substituted C₁-C₆ alkyl, or R^{26a} and R^{26b}, together with the atom to which each is attached, combine to form

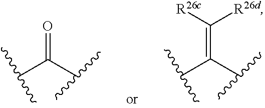

where each of R^{26a} and R²⁶ is, independently, H or optionally substituted C₁-C₆ alkyl; and

each of R^{27a} and R^{27b} is H, hydroxyl, or optionally substituted C₁-C₆ alkyl, or a pharmaceutically acceptable salt thereof.

In some embodiments, the compound has the structure of Formula SVIIa:

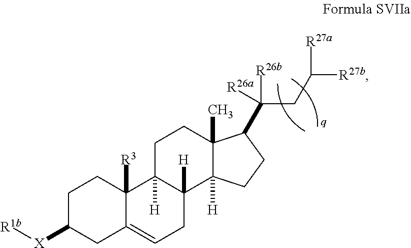

or a pharmaceutically acceptable salt thereof.

In some embodiments, the compound has the structure of Formula SVIIb:

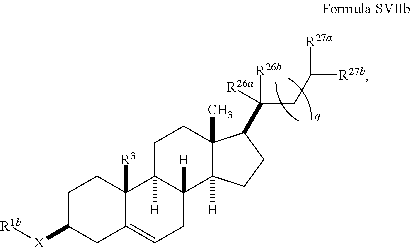

or a pharmaceutically acceptable salt thereof.

In some embodiments, R^{26a} and R^{26b} is, independently, H,

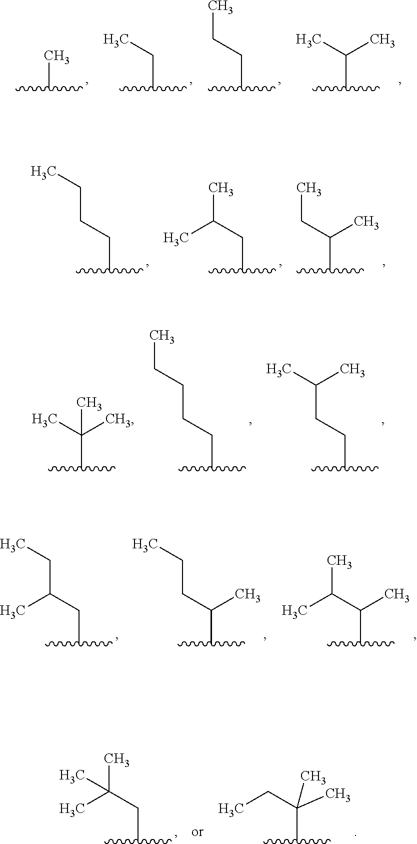

In some embodiments, R^{26a} and R^{26b}, together with the atom to which each is attached, combine to form

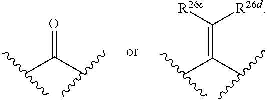

In some embodiments, R^{26a} and R^{26b}, together with the atom to which each is attached, combine to form

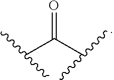 [SMILES: *C(*)=O]

In some embodiments, R^{26a} and R^{26b}, together with the atom to which each is attached, combine to form

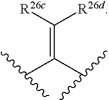

In some embodiments, where each of R^{26c} and R²⁶ is, independently, H,

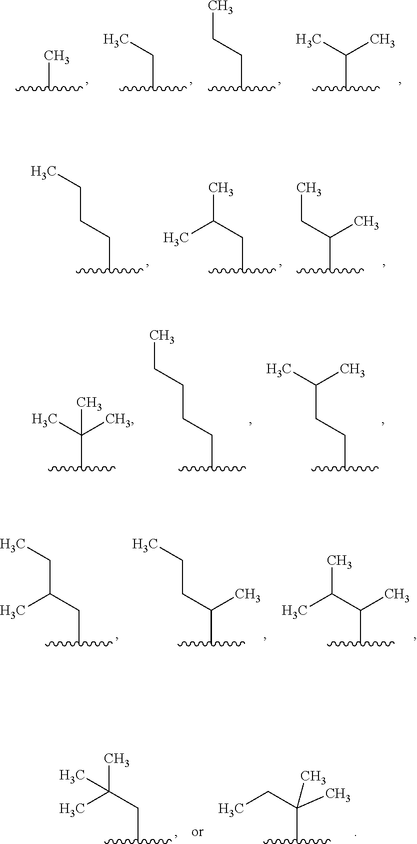

In some embodiments, each of R^{27a} and R^{27b} is H, hydroxyl, or optionally substituted C₁-C₃ alkyl.

In some embodiments, each of R^{27a} and R^{27b} is, independently, H, hydroxyl,

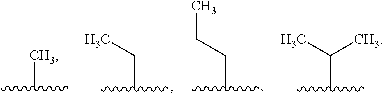

In an aspect, the structural lipid of the disclosure features a compound having the structure of Formula SVIII:

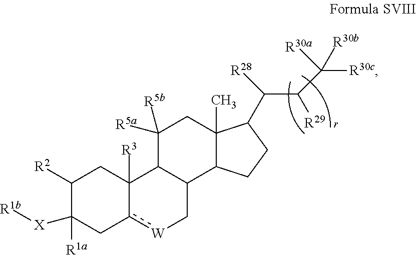

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

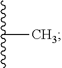

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

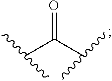 [SMILES: *C(*)=O]

R²⁸ is H or optionally substituted C₁-C₆ alkyl;

r is 1, 2, or 3;

each R²⁹ is, independently, H or optionally substituted C₁-C₆ alkyl; and

each of R^{30a}, R^{30b}, and R^{30c} is C₁-C₆ alkyl,

or a pharmaceutically acceptable salt thereof.

In some embodiments, the compound has the structure of Formula SVIIIa:

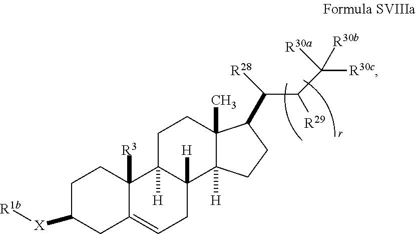

or a pharmaceutically acceptable salt thereof.

In some embodiments, the compound has the structure of Formula SVIIIb:

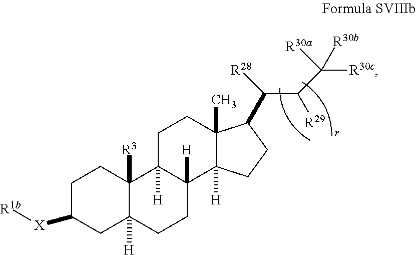

or a pharmaceutically acceptable salt thereof.

In some embodiments, R²⁸ is H,

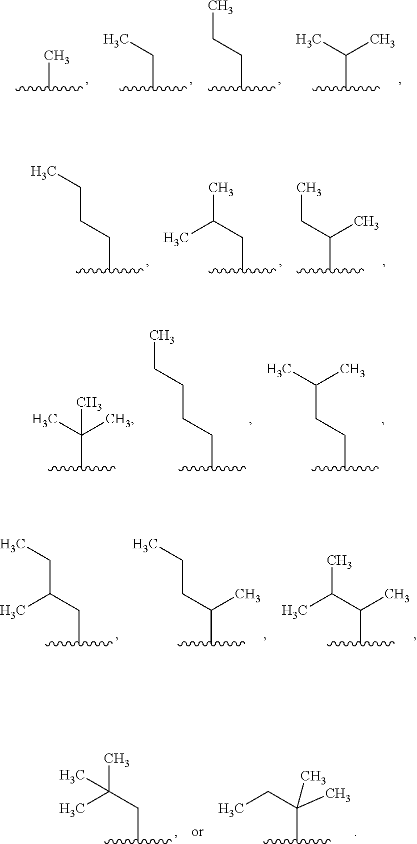

In some embodiments, R²⁸ is

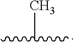

In some embodiments, each of R^{30a}, R^{30b}, and R^{30c} is, independently,

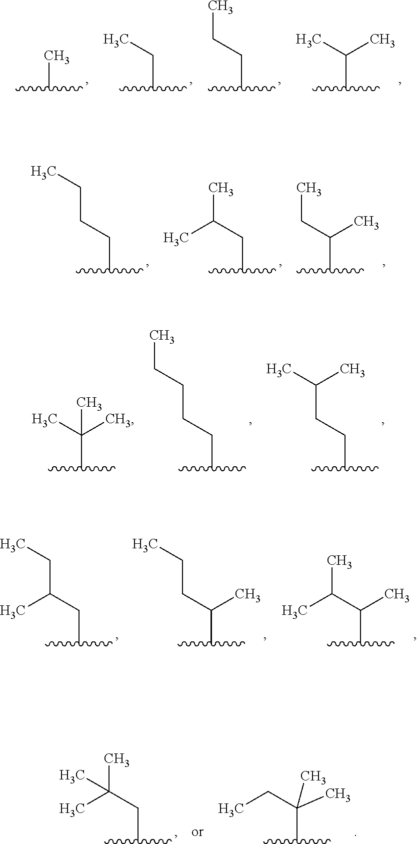

In some embodiments, r is 1. In some embodiments, r is 2. In some embodiments, r is 3.

In some embodiments, each R²⁹ is, independently, H,

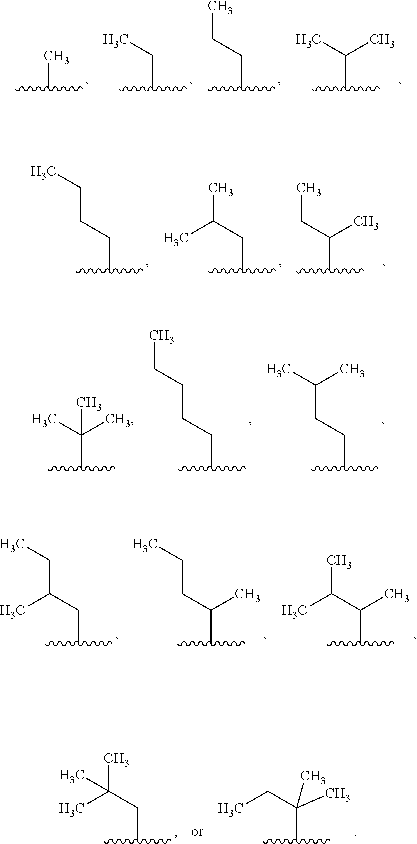

In some embodiments, each R²⁹ is, independently, H or

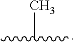

In an aspect, the structural lipid of the disclosure features a compound having the structure of Formula SIX:

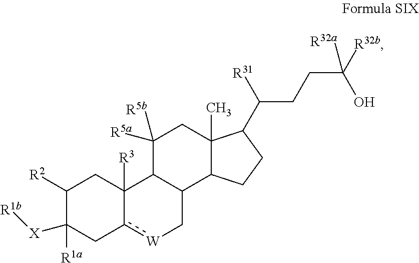

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

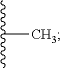

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

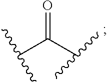 [SMILES: *C(*)=O]

R³¹ is H or C₁-C₆ alkyl; and

each of R^{32a} and R^{32b} is C₁-C₆ alkyl,

or a pharmaceutically acceptable salt thereof.

In some embodiments, the compound has the structure of Formula SIXa:

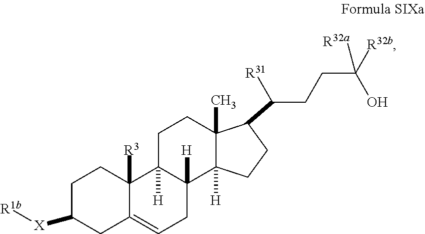

or a pharmaceutically acceptable salt thereof.

In some embodiments, the compound has the structure of Formula SIXb:

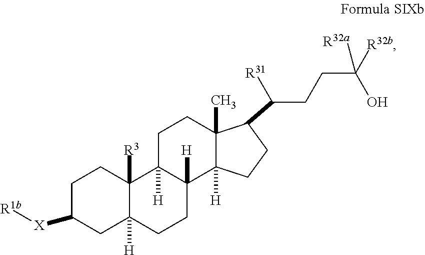

or a pharmaceutically acceptable salt thereof.

In some embodiments, R³¹ is H,

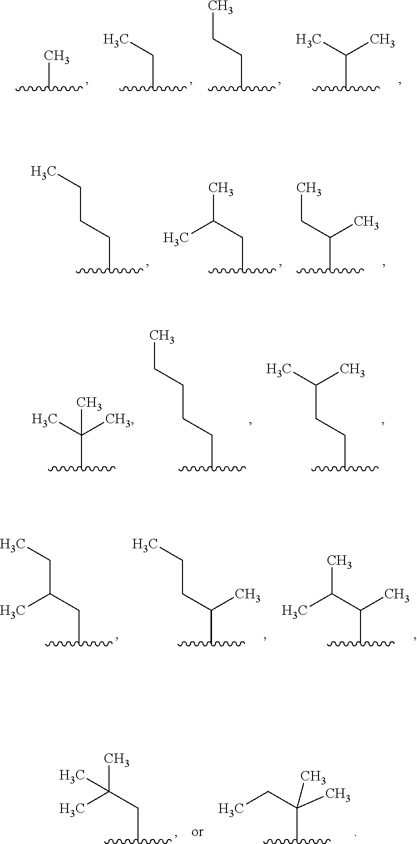

In some embodiments, R³¹ is

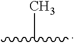

In some embodiments, each of R^{32a} and R^{32b} is, independently,

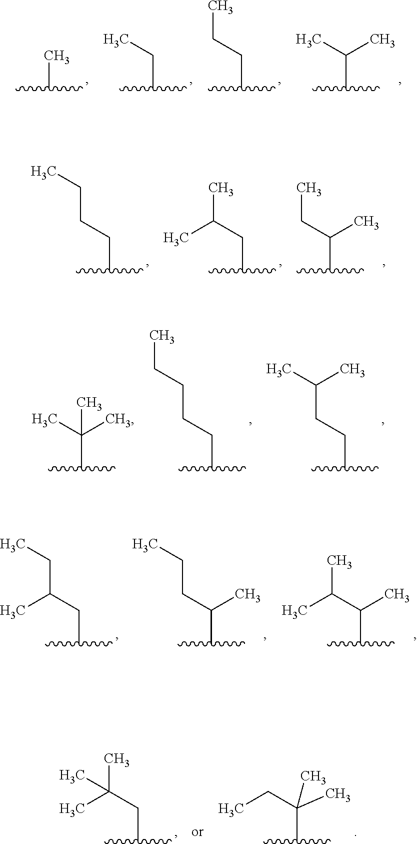

In an aspect, the structural lipid of the disclosure features a compound having the structure of Formula SX:

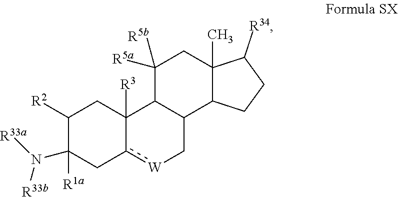

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

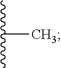

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

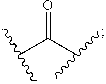 [SMILES: *C(*)=O]

R³³a is optionally substituted C₁-C₆ alkyl or

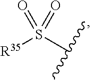

where R³⁵ is optionally substituted C₁-C₆ alkyl or optionally substituted C₆-C₁₀ aryl;

R^{33b} is H or optionally substituted C₁-C₆ alkyl; or

R³⁵ and R^{33b}, together with the atom to which each is attached, form an optionally substituted C₃-C₉ heterocyclyl; and

R³⁴ is optionally substituted C₁-C₆ alkyl or optionally substituted C₁-C₆ heteroalkyl, or a pharmaceutically acceptable salt thereof.

In some embodiments, the compound has the structure of Formula SXa:

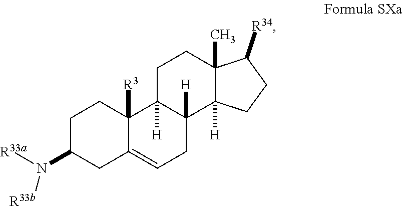

or a pharmaceutically acceptable salt thereof.

In some embodiments, the compound has the structure of Formula SXb:

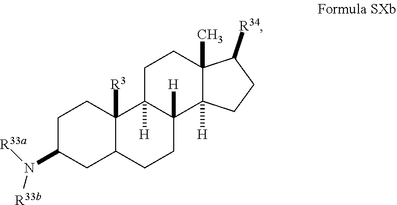

or a pharmaceutically acceptable salt thereof.

In some embodiments, R³³a is

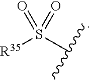

In some embodiments, R³⁵ is

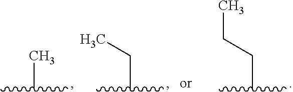

In some embodiments, R³⁵ is

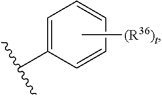

where

t is 0, 1, 2, 3, 4, or 5; and

each R³⁶ is, independently, halo, hydroxyl, optionally substituted C₁-C₆ alkyl, or optionally substituted C₁-C₆ heteroalkyl.

In some embodiments, R³⁴ is

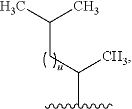

where u is 0, 1, 2, 3, or 4.

In some embodiments, u is 3 or 4.

In an aspect, the structural lipid of the disclosure features a compound having the structure of Formula SXI:

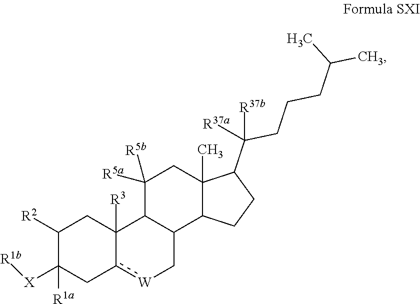

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

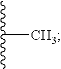

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

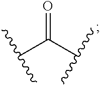 [SMILES: *C(*)=O]

and

each of R^{37a} and R^{37b} is, independently, optionally substituted C₁-C₆ alkyl, optionally substituted C₁-C₆ heteroalkyl, halo, or hydroxyl,

or a pharmaceutically acceptable salt thereof.

In some embodiments, the compound has the structure of Formula SXIa:

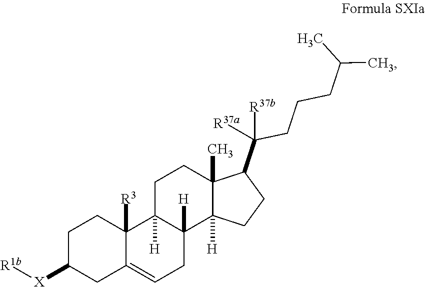

or a pharmaceutically acceptable salt thereof.

In some embodiments, the compound has the structure of Formula SXIb:

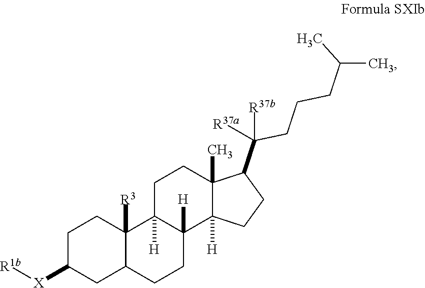

or a pharmaceutically acceptable salt thereof.

In some embodiments, R³⁷a is hydroxyl.

In some embodiments, R^{37b} is

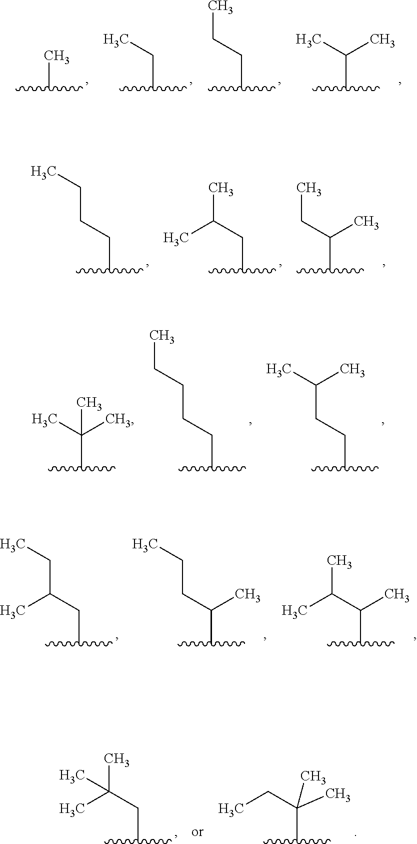

In an aspect, the structural lipid of the disclosure features a compound having the structure of Formula SXII:

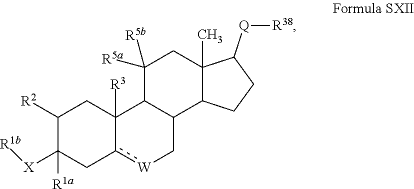

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

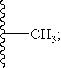

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form and

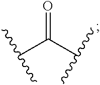 [SMILES: *C(*)=O]

Q is 0, S, or NR^{E}, where R^{E} is H or optionally substituted C₁-C₆ alkyl; and

R³⁸ is optionally substituted C₁-C₆ alkyl,

or a pharmaceutically acceptable salt thereof.

In some embodiments, the compound has the structure of Formula SXIIa:

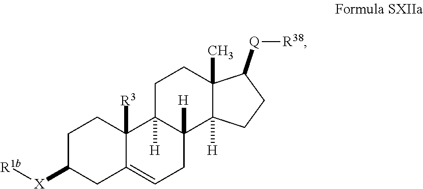

or a pharmaceutically acceptable salt thereof.

In some embodiments, the compound has the structure of Formula SXIIb:

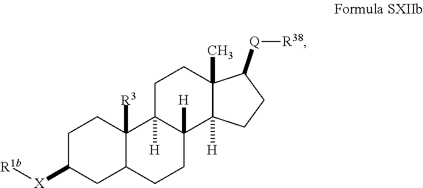

or a pharmaceutically acceptable salt thereof.

In some embodiments, Q is NR^{E}.

In some embodiments, R^{E} is H or

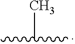

In some embodiments, R^{E} is H. In some embodiments, R^{E} is

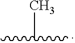

In some embodiments, R³⁸ is

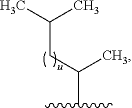

where u is 0, 1, 2, 3, or 4.

In some embodiments, X is O.

In some embodiments, R^{1a} is H or optionally substituted C₁-C₆ alkyl.

In some embodiments, R^{1a} is H.

In some embodiments, R^{1b} is H or optionally substituted C₁-C₆ alkyl.

In some embodiments, R^{1b} is H.

In some embodiments, R² is H.

In some embodiments, R^{4a} is H.

In some embodiments, R^{4b} is H.

In some embodiments, [symbol] represents a double bond.

In some embodiments, R³ is H. In some embodiments, R³ is

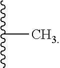

In some embodiments, R^{5a} is H.

In some embodiments, R^{5b} is H.

In an aspect, the disclosure features a compound having the structure of any one of compounds S-1-42, S-150, S-154, S-162-165, S-169-172 and S-184 in Table 1A, or any pharmaceutically acceptable salt thereof. As used herein, “CMPD” refers to “compound.”

TABLE 1A
Compounds of Formula SI

| CMPD | |
|---|---|
| No. S- | Structure |
| 1 | 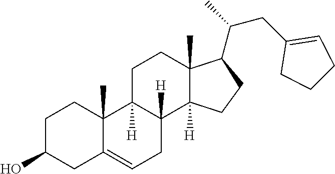 |
| 2 | 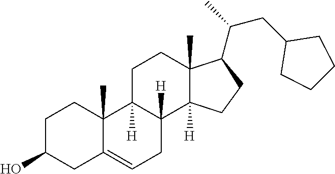 |
| 3 | 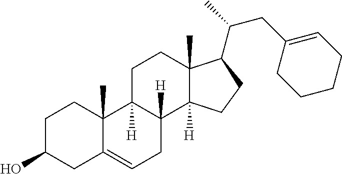 |
| 4 | 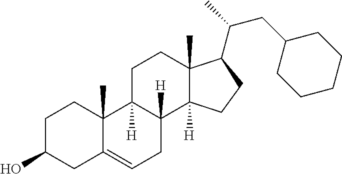 |
| 5 | 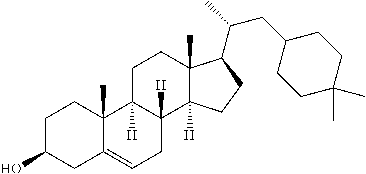 |
| 6 | 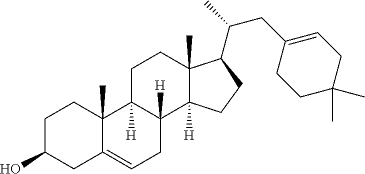 |
| 7 | 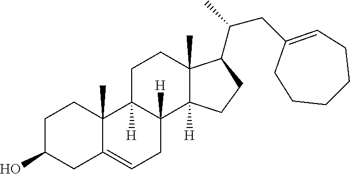 |
| 8 | 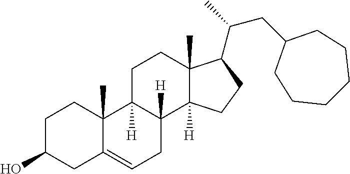 |
| 9 | 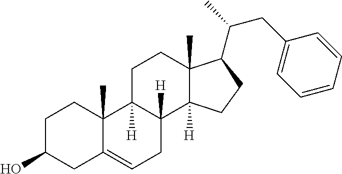 |
| 10 | 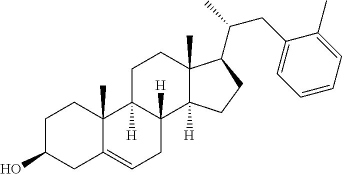 |
| 11 | 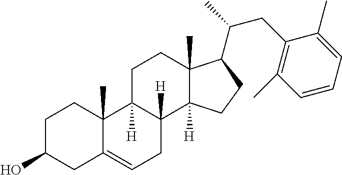 |
| 12 | 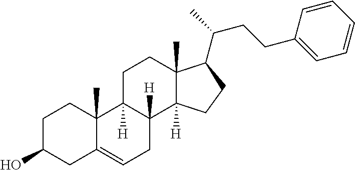 |
| 13 | 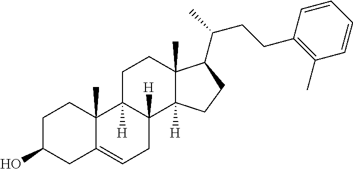 |
| 14 | 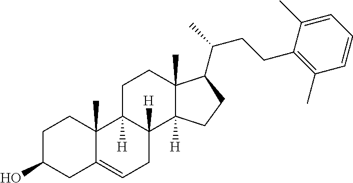 |
| 15 | 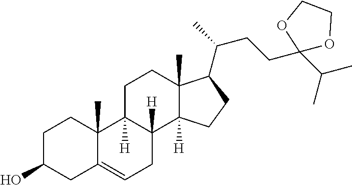 |
| 16 | 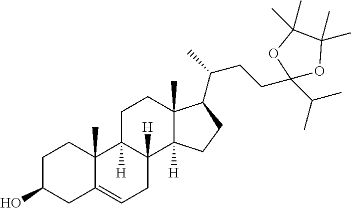 |
| 17 | 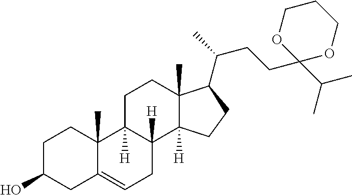 |
| 18 | 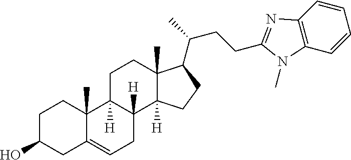 |
| 19 | 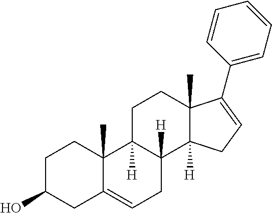 |
| 20 | 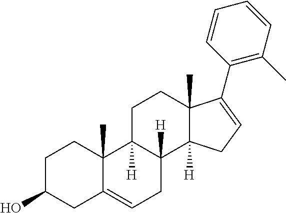 |
| 21 | 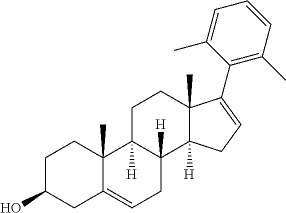 |
| 150 | 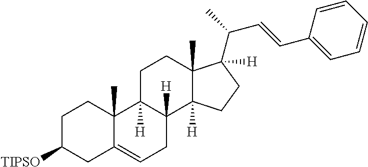 |
| 154 | 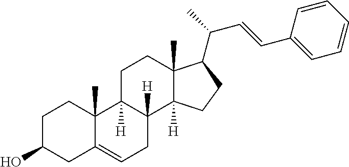 |
| 162 | 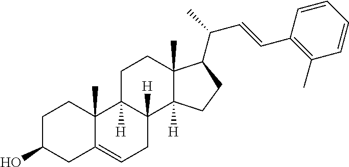 |
| 163 | 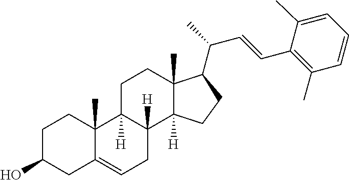 |
| 164 | 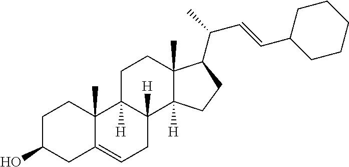 |
| 184 | 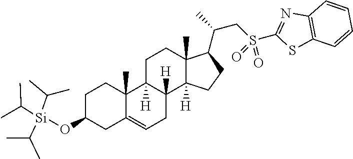 |
| 22 | 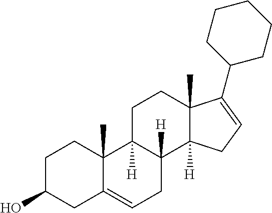 |
| 23 | 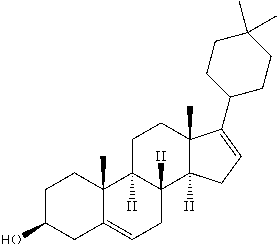 |
| 24 | 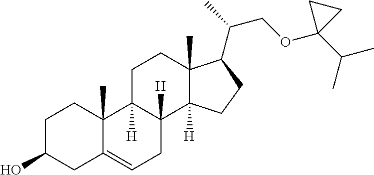 |
| 25 | 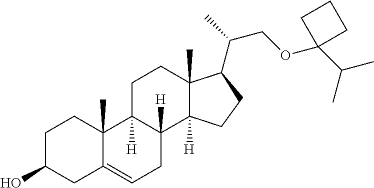 |
| 26 | 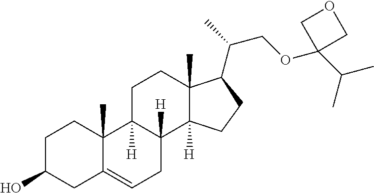 |
| 27 | 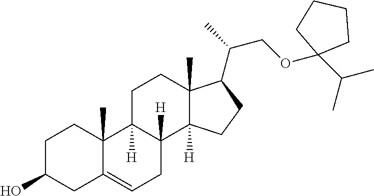 |
| 28 | 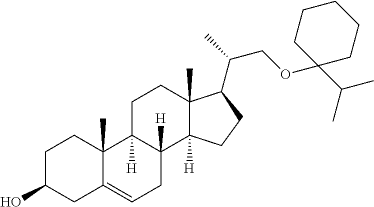 |
| 29 | 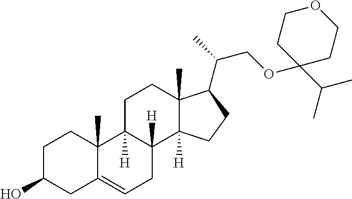 |
| 30 | 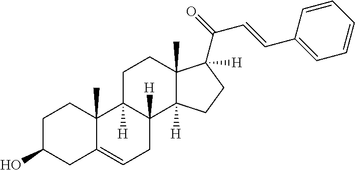 |
| 31 | 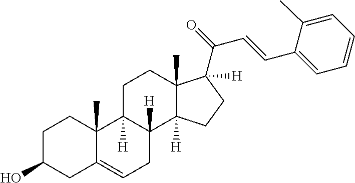 |
| 32 | 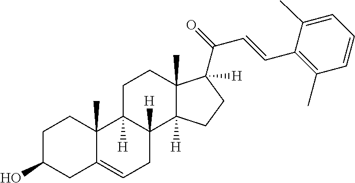 |
| 33 | 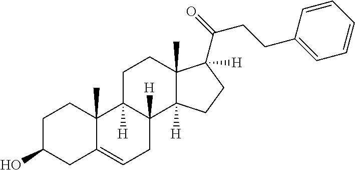 |
| 34 | 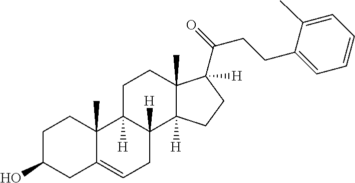 |
| 35 | 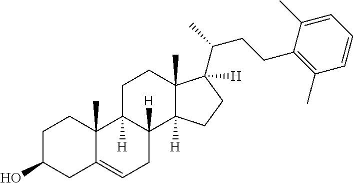 |
| 36 | 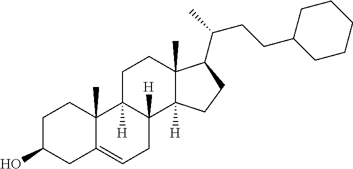 |
| 37 | 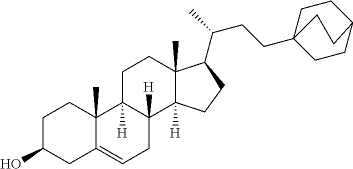 |
| 38 | 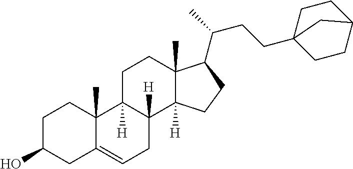 |
| 39 | 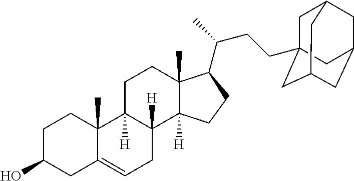 |
| 40 | 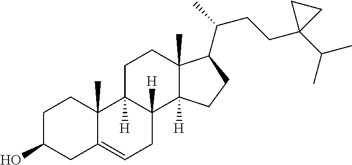 |
| 41 | 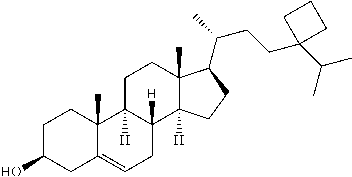 |
| 42 | 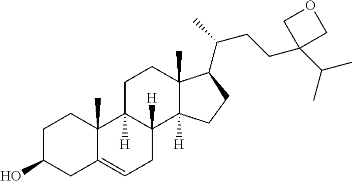 |
| 165 | 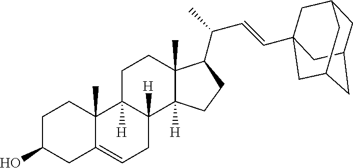 |
| 169 | 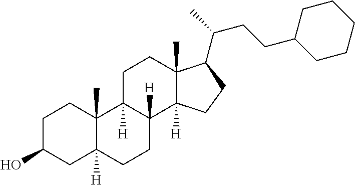 |
| 170 | 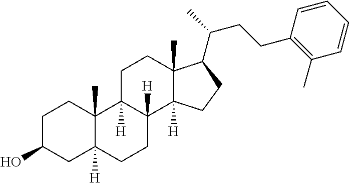 |
| 171 | 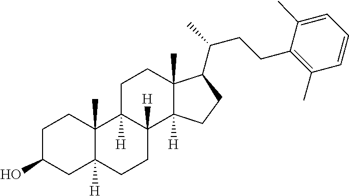 |
| 172 | 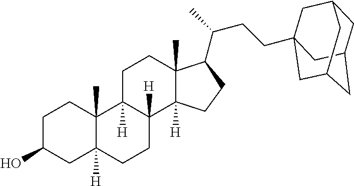 |

In an aspect, the disclosure features a compound having the structure of any one of compounds S-43-50 and S-175-178 in Table 2, or any pharmaceutically acceptable salt thereof.

TABLE 2
Compounds of Formula SII

| CMPD | |
|---|---|
| No. S- | Structure |
| 43 | 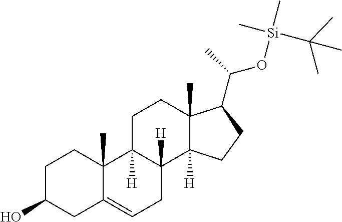 |
| 44 | 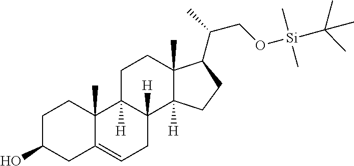 |
| 45 | 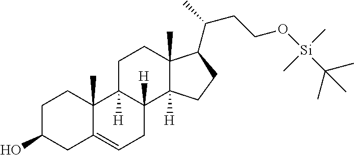 |
| 46 | 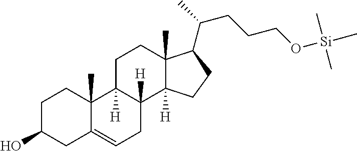 |
| 175 | 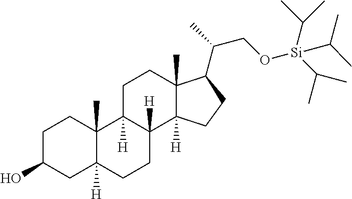 |
| 176 | 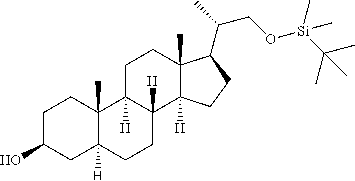 |
| 47 | 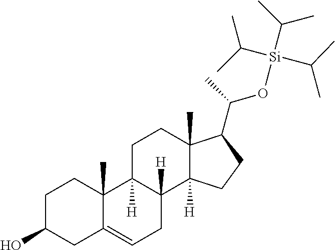 |
| 48 | 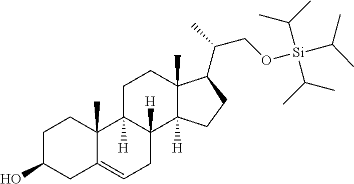 |
| 49 | 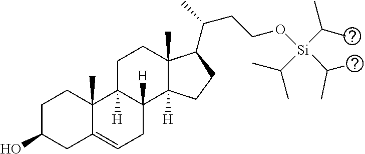 |
| 50 | 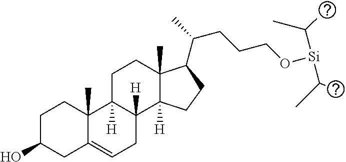 |
| 177 | 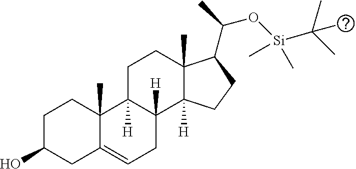 |
| 178 | 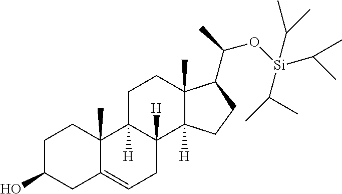 |

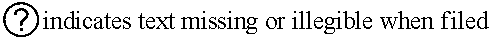

In an aspect, the disclosure features a compound having the structure of any one of compounds S-51-67, S-149 and S-153 in Table 3, or any pharmaceutically acceptable salt thereof.

TABLE 3
Compounds of Formula SIII

| CMPD | |
|---|---|
| No. S- | Structure |
| 51 | 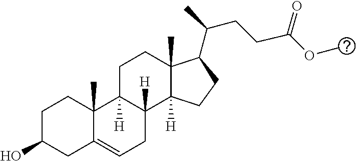 |
| 52 | 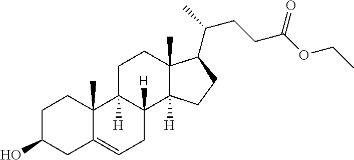 |
| 53 | 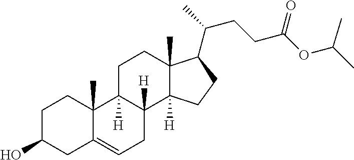 |
| 54 | 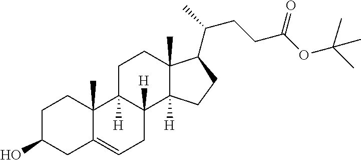 |
| 55 | 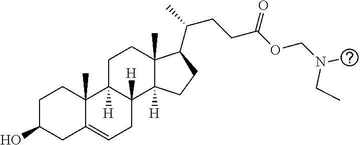 |
| 56 | 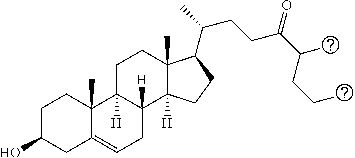 |
| 57 | 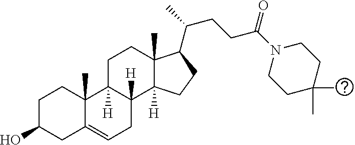 |
| 58 | 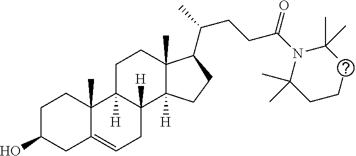 |
| 59 | 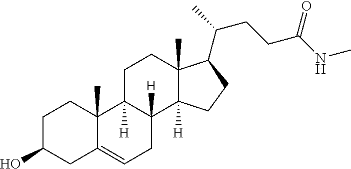 |
| 153 | 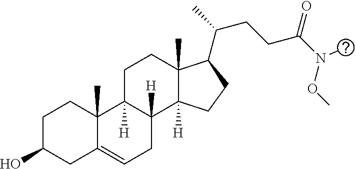 |
| 60 | 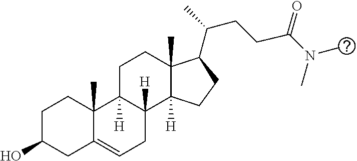 |
| 61 | 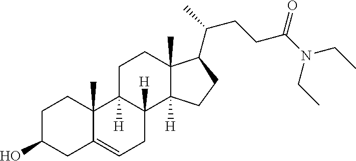 |
| 62 | 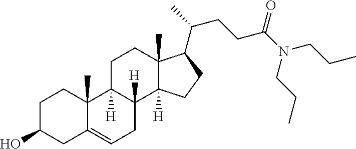 |
| 63 | 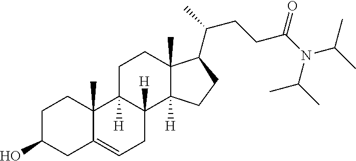 |
| 64 | 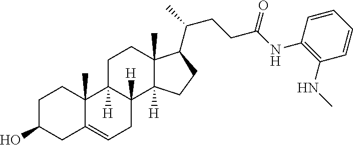 |
| 65 | 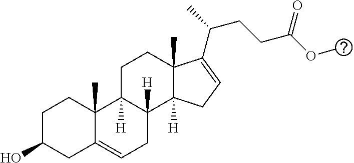 |
| 66 | 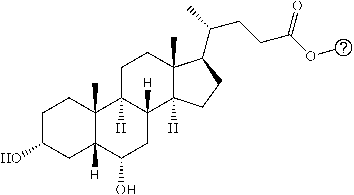 |
| 67 | 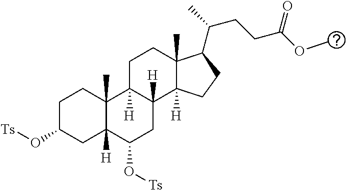 |
| 149 | 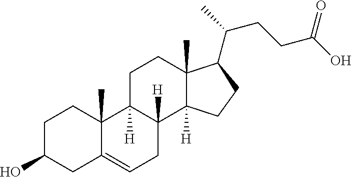 |

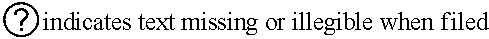

In an aspect, the disclosure features a compound having the structure of any one of compounds S-68-73 in Table 4, or any pharmaceutically acceptable salt thereof.

TABLE 4
Compounds of Formula SIV

| CMPD | |
|---|---|
| No. S- | Structure |
| 68 | 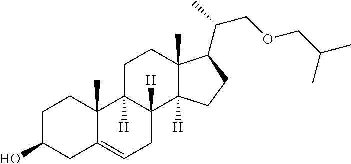 |
| 69 | 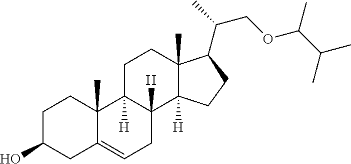 |
| 70 | 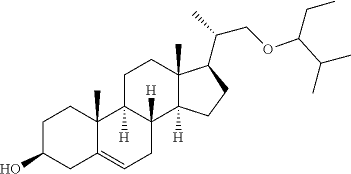 |
| 71 | 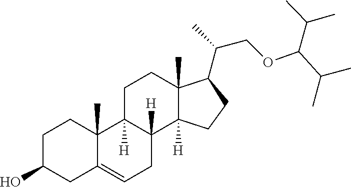 |
| 72 | 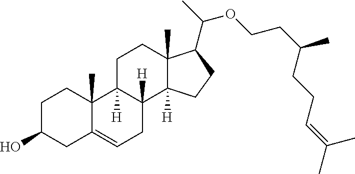 |
| 73 | 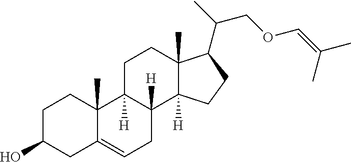 |

In an aspect, the disclosure features a compound having the structure of any one of compounds S-74-78 in Table 5, or any pharmaceutically acceptable salt thereof.

TABLE 5
Compounds of Formula SV

| CMPD | |
|---|---|
| No. S- | Structure |
| 74 | 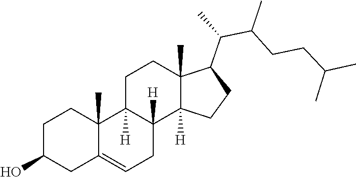 |
| 75 | 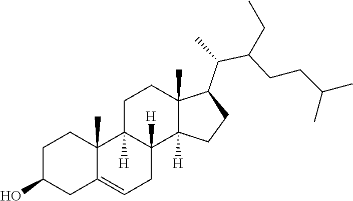 |
| 76 | 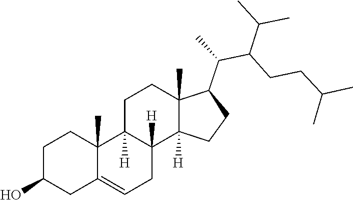 |
| 77 | 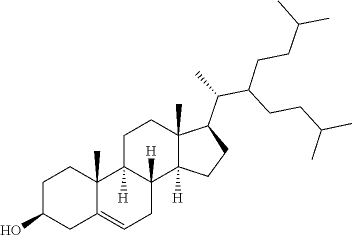 |
| 78 | 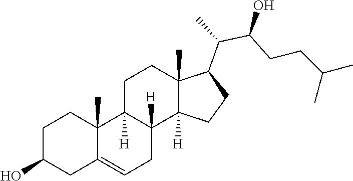 |

In an aspect, the disclosure features a compound having the structure of any one of compounds S-79 or S-80 in Table 6, or any pharmaceutically acceptable salt thereof.

TABLE 6
Compounds of Formula SVI

| CMPD | |
|---|---|
| No. S- | Structure |
| 79 | 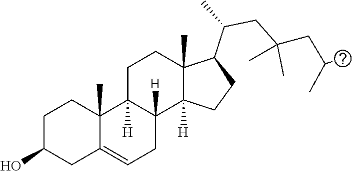 |
| 80 | 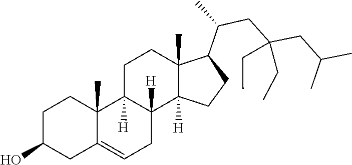 |

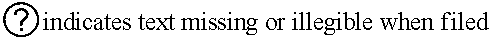

In an aspect, the disclosure features a compound having the structure of any one of compounds S-81-87, S-152 and S-157 in Table 7, or any pharmaceutically acceptable salt thereof.

TABLE 7
Compounds of Formula S-VII

| CMPD | |
|---|---|
| No. S- | Structure |
| 81 | 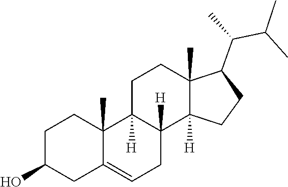 |
| 82 | 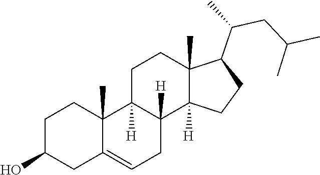 |
| 83 | 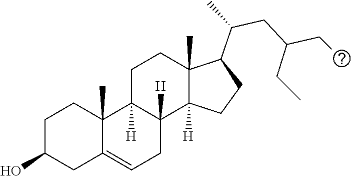 |
| 84 | 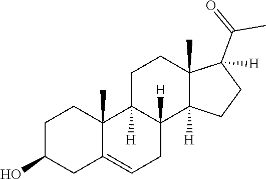 |
| 157 | 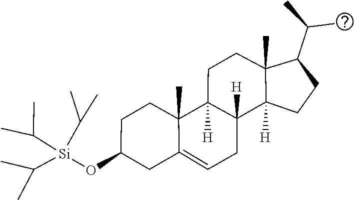 |
| 85 | 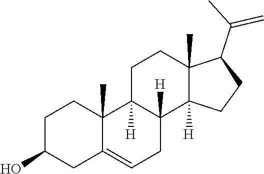 |
| 86 | 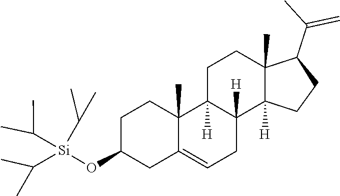 |
| 87 | 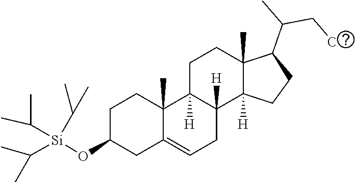 |
| 152 | 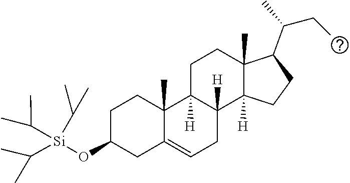 |

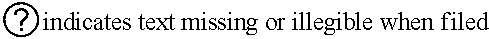

In an aspect, the disclosure features a compound having the structure of any one of compounds S-88-97 in Table 8, or any pharmaceutically acceptable salt thereof.

TABLE 8
Compounds of Formula SVIII

| CMPD | |
|---|---|
| No. S- | Structure |
| 88 | 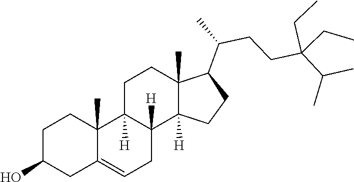 |
| 89 | 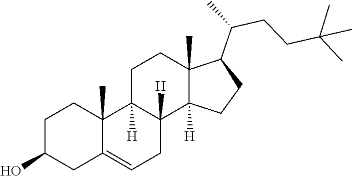 |
| 90 | 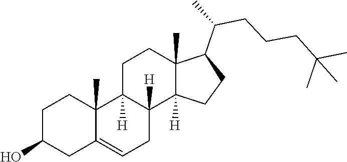 |
| 91 | 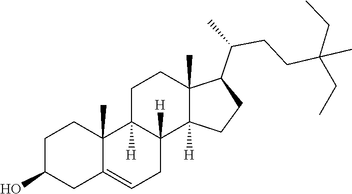 |
| 92 | 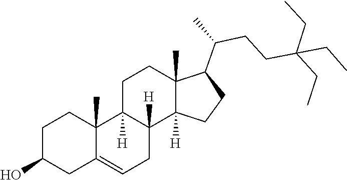 |
| 93 | 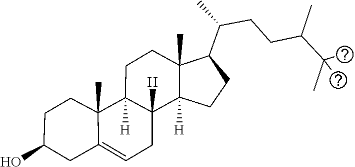 |
| 94 | 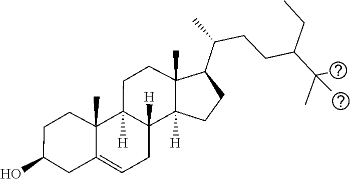 |
| 95 | 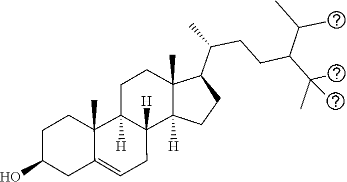 |
| 96 | 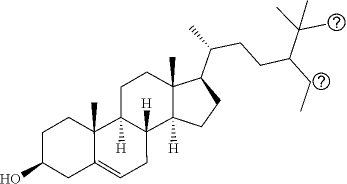 |
| 97 | 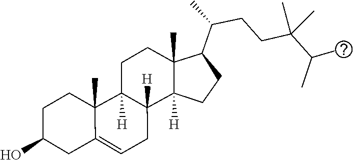 |

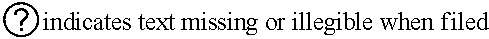

In an aspect, the disclosure features a compound having the structure of any one of compounds S-98-105 and S-180-182 in Table 9, or any pharmaceutically acceptable salt thereof.

TABLE 9
Compounds of Formula SIX

| CMPD | |
|---|---|
| No. S- | Structure |
| 98 | 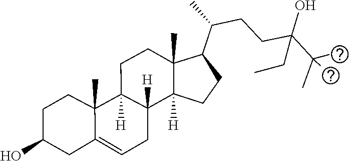 |
| 99 | 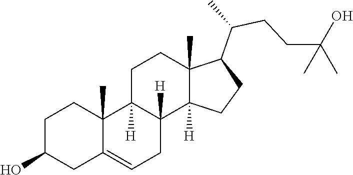 |
| 100 | 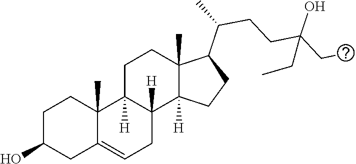 |
| 101 | 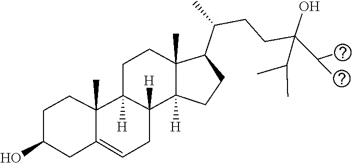 |
| 180 | 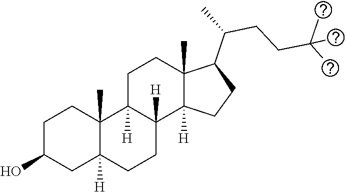 |
| 181 | 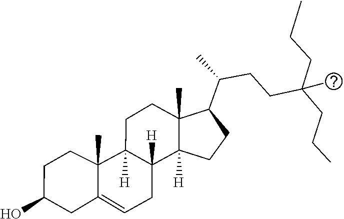 |
| 102 | 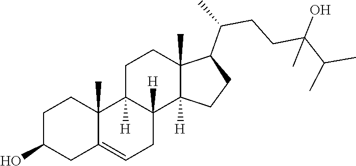 |
| 103 | 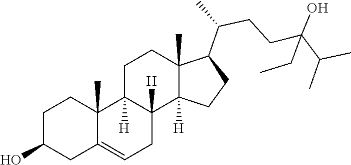 |
| 104 | 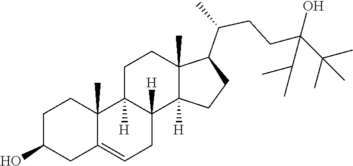 |
| 105 | 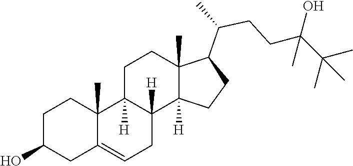 |
| 182 | 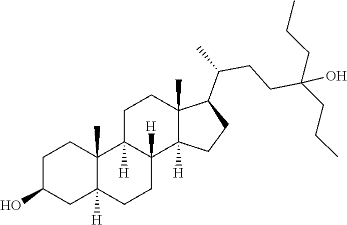 |

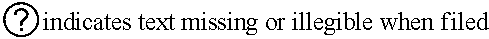

In an aspect, the disclosure features a compound having the structure of compound S-106 in Table 10, or any pharmaceutically acceptable salt thereof.

TABLE 10
Compounds of Formula SX

| CMPD | |
|---|---|
| No. S- | Structure |
| 106 | 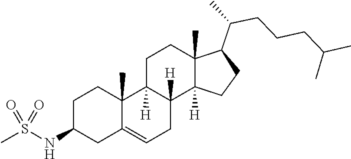 |

In an aspect, the disclosure features a compound having the structure of compound S-107 or S-108 in Table 11, or any pharmaceutically acceptable salt thereof.

TABLE 11
Compounds of Formula SXI

| CMPD | |
|---|---|
| No. S- | Structure |
| 107 | 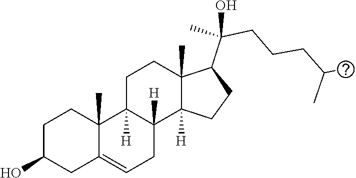 |
| 108 | 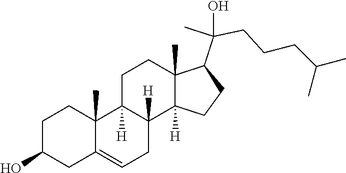 |

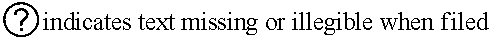

In an aspect, the disclosure features a compound having the structure of compound S-109 in Table 12, or any pharmaceutically acceptable salt thereof.

TABLE 12
Compounds of Formula SXII

| CMPD | |
|---|---|
| No. S- | Structure |
| 109 | 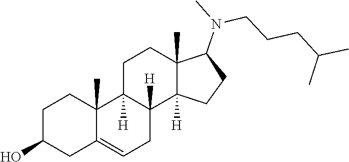 |

In an aspect, the disclosure features a compound having the structure of any one of compounds S-110-130, S-155, S-156, S-158, S-160, S-161, S-166-168, S-173, S-174 and S-179 in Table 13, or any pharmaceutically acceptable salt thereof.

TABLE 13
Compounds of the Disclosure

| CMPD | |
|---|---|
| No. S- | Structure |
| 110 | 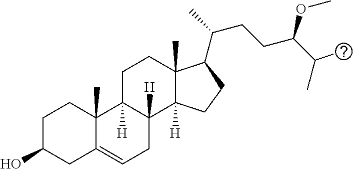 |
| 111 | 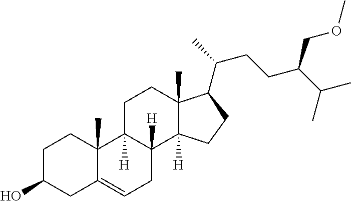 |
| 112 | 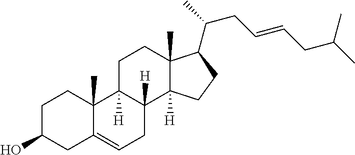 |
| 113 | 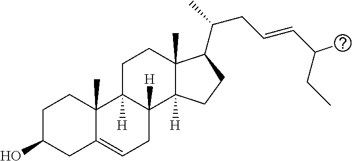 |
| 114 | 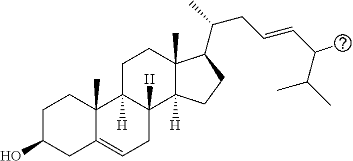 |
| 115 | 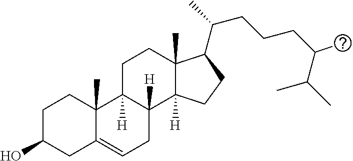 |
| 116 | 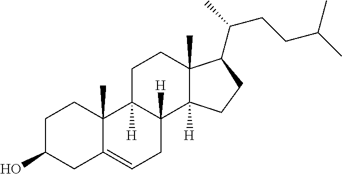 |
| 117 | 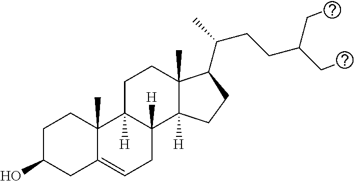 |
| 118 | 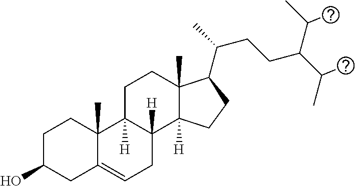 |
| 119 | 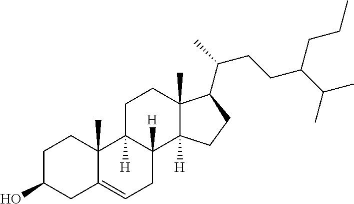 |
| 120 | 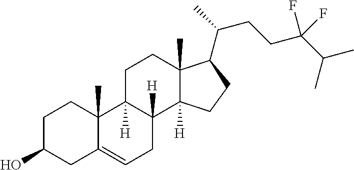 |
| 156 | 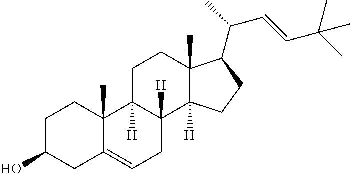 |
| 158 | 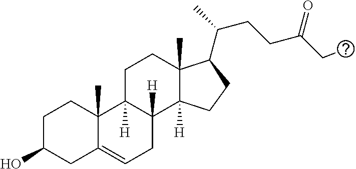 |
| 160 | 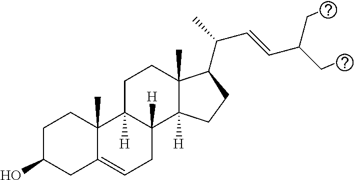 |
| 161 | 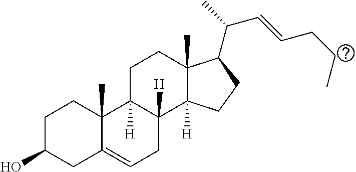 |
| 166 | 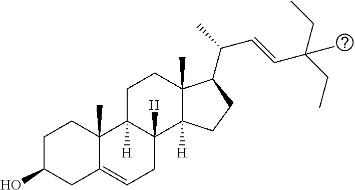 |
| 121 | 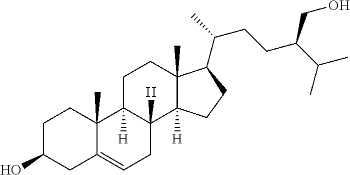 |
| 122 | 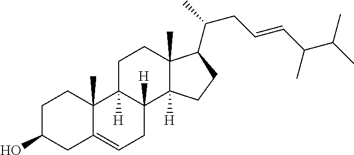 |
| 123 | 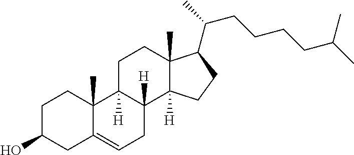 |
| 124 | 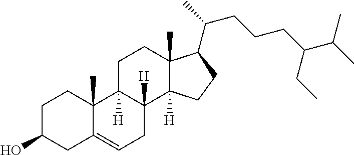 |
| 125 | 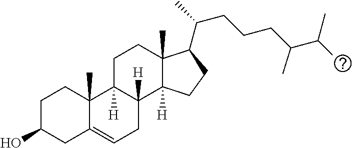 |
| 126 | 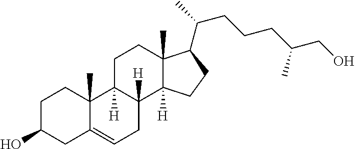 |
| 127 | 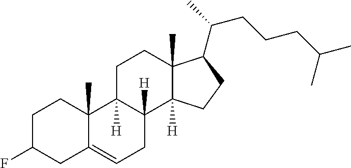 |
| 128 | 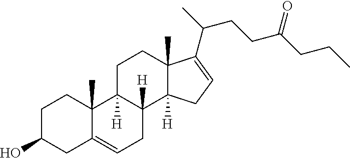 |
| 129 | 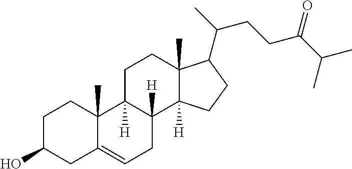 |
| 130 | 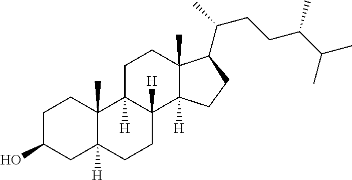 |
| 155 | 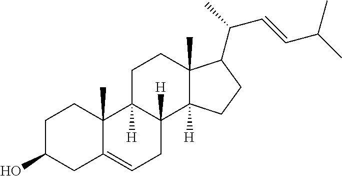 |
| 167 | 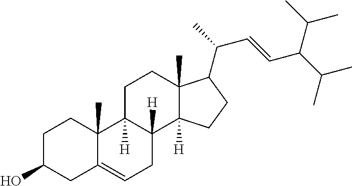 |
| 168 | 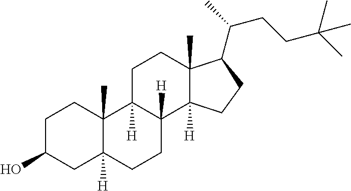 |
| 173 | 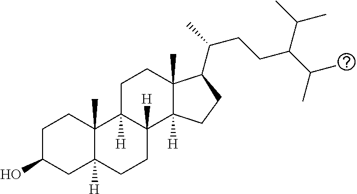 |
| 174 | 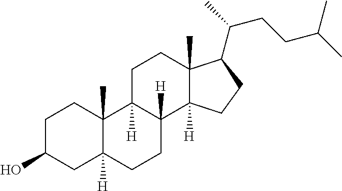 |
| 179 | 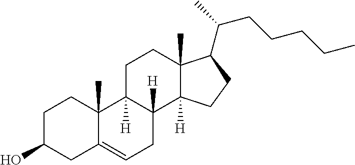 |

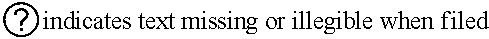

In an aspect, the disclosure features a compound having the structure of any one of compounds S-131-133 in Table 14, or any pharmaceutically acceptable salt thereof.

TABLE 14
Compounds of the Disclosure

| CMPD | |
|---|---|
| No. S- | Structure |
| 131 | 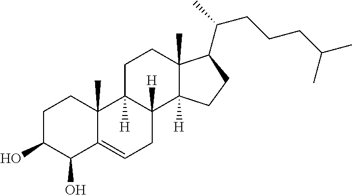 |
| 132 | 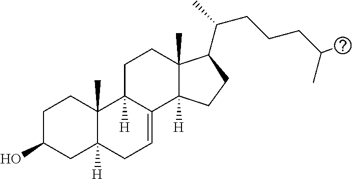 |
| 133 | 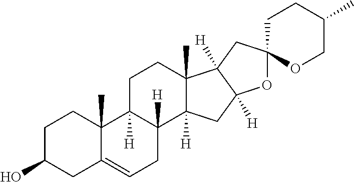 |

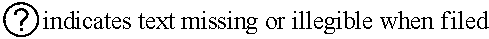

In an aspect, the disclosure features a compound having the structure of any one of compounds S-134-148, S-151 and S-159 in Table 15, or any pharmaceutically acceptable salt thereof.

TABLE 15
Compounds of the Disclosure

| CMPD | |
|---|---|
| No. S- | Structure |
| 134 | 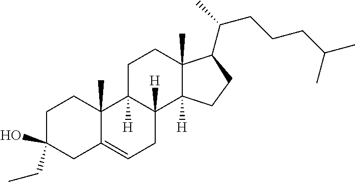 |
| 135 | 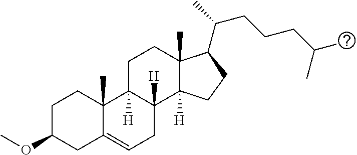 |
| 136 | 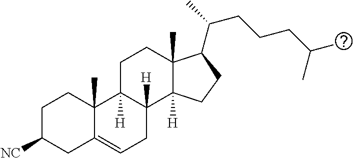 |
| 137 | 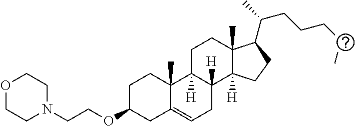 |
| 138 | 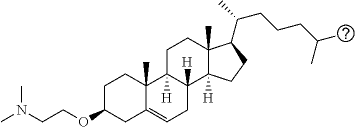 |
| 139 | 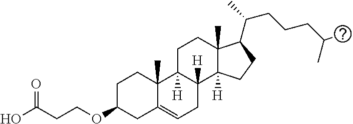 |
| 140 | 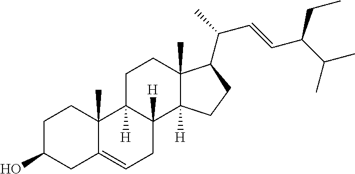 |
| 141 | 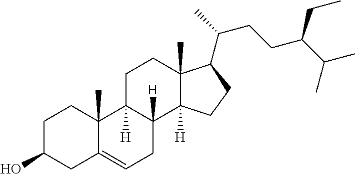 |
| 159 | 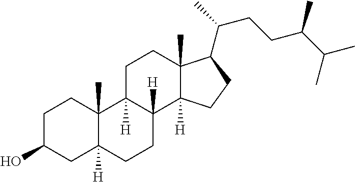 |
| 142 | 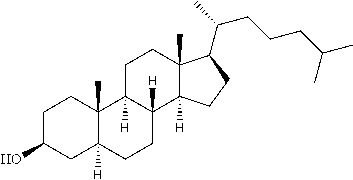 |
| 143 | 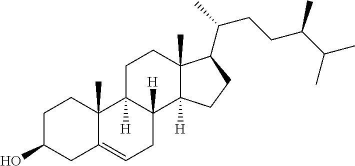 |
| 144 | 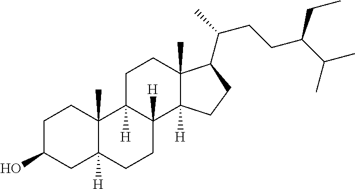 |
| 145 | 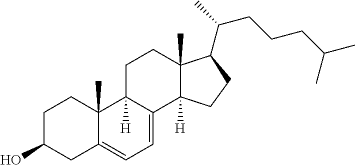 |
| 146 | 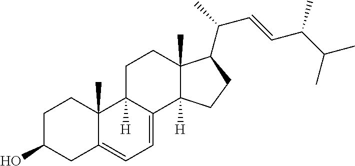 |
| 147 | 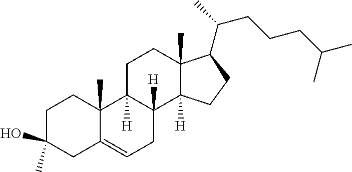 |
| 148 | 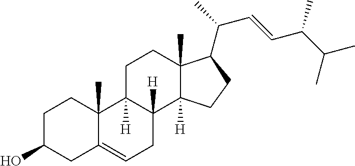 |
| 151 | 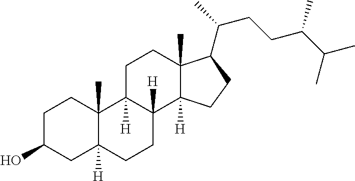 |

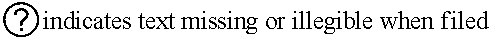

The one or more structural lipids of the lipid nanoparticles of the disclosure can be a composition of structural lipids (e.g., a mixture of two or more structural lipids, a mixture of three or more structural lipids, a mixture of four or more structural lipids, or a mixture of five or more structural lipids). A composition of structural lipids can include, but is not limited to, any combination of sterols (e.g., cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds 134-148, 151, and 159 in Table 15). For example, the one Or more structural lipids of the lipid nanoparticles of the disclosure can be composition 183 in Table 16.

TABLE 16
Structural Lipid Compositions

| Composi- | |
|---|---|
| tion S-No. | Structure |
| 183 | 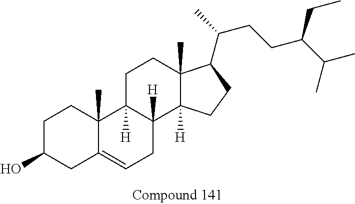 |
| | 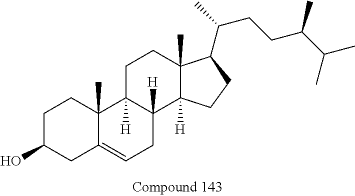 |
| | 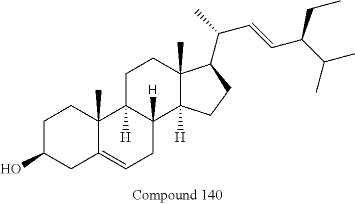 |
| | 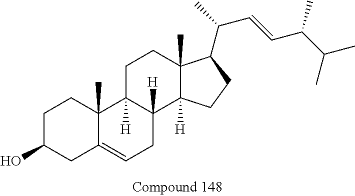 |

Composition S-183 is a mixture of compounds S-141, S-140, S-143, and S-148. In some embodiments, composition S-183 includes about 35% to about 45% of compound S-141, about 20% to about 30% of compound S-140, about 20% to about 30% compound S-143, and about 5% to about 15% of compound S-148. In some embodiments, composition 183 includes about 40% of compound S-141, about 25% of compound S-140, about 25% compound S-143, and about 10% of compound S-148.

In some embodiments, the structural lipid is a pytosterol. In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

Ratio of Compounds

A lipid nanoparticle of the disclosure can include a structural component as described herein. The structural component of the lipid nanoparticle can be any one of compounds S-1-148, a mixture of one or more structural compounds of the disclosure and/or any one of compounds S-1-148 combined with a cholesterol and/or a phytosterol.

For example, the structural component of the lipid nanoparticle can be a mixture of one or more structural compounds (e.g. any of Compounds S-1-148) of the disclosure with cholesterol. The mol % of the structural compound present in the lipid nanoparticle relative to cholesterol can be from 0-99 mol %. The mol % of the structural compound present in the lipid nanoparticle relative to cholesterol can be about 10 mol %, 20 mol %, 30 mol %, 40 mol %, 50 mol %, 60 mol %, 70 mol %, 80 mol %, or 90 mol %.

In one aspect, the disclosure features a composition including two or more sterols, wherein the two or more sterols include at least two of: β-sitosterol, sitostanol, camesterol, stigmasterol, and brassicasteol. The composition may additionally comprise cholesterol. In one embodiment, β-sitosterol comprises about 35-99%, e.g., about 40%, 50%, 60%, 70%, 80%, 90%, 95% or greater of the non-cholesterol sterol in the composition.

In another aspect, the disclosure features a composition including two or more sterols, wherein the two or more sterols include β-sitosterol and campesterol, wherein β-sitosterol includes 95-99.9% of the sterols in the composition and campesterol includes 0.1-5% of the sterols in the composition.

In some embodiments, the composition further includes sitostanol. In some embodiments, β-sitosterol includes 95-99.9%, campesterol includes 0.05-4.95%, and sitostanol includes 0.05-4.95% of the sterols in the composition.

In another aspect, the disclosure features a composition including two or more sterols, wherein the two or more sterols include β-sitosterol and sitostanol, wherein β-sitosterol includes 95-99.9% of the sterols in the composition and sitostanol includes 0.1-5% of the sterols in the composition.

In some embodiments, the composition further includes campesterol. In some embodiments, β-sitosterol includes 95-99.9%, campesterol includes 0.05-4.95%, and sitostanol includes 0.05-4.95% of the sterols in the composition.

In some embodiments, the composition further includes campesterol. In some embodiments, β-sitosterol includes 75-80%, campesterol includes 5-10%, and sitostanol includes 10-15% of the sterols in the composition.

In some embodiments, the composition further includes an additional sterol. In some embodiments, β-sitosterol includes 35-45%, stigmasterol includes 20-30%, and campesterol includes 20-30%, and brassicasterol includes 1-5% of the sterols in the composition.

In another aspect, the disclosure features a composition including a plurality of lipid nanoparticles, wherein the plurality of lipid nanoparticles include an ionizable lipid and two or more sterols, wherein the two or more sterols include β-sitosterol, and campesterol and β-sitosterol includes 95-99.9% of the sterols in the composition and campesterol includes 0.1-5% of the sterols in the composition.

In some embodiments, the two or more sterols further includes sitostanol. In some embodiments, β-sitosterol includes 95-99.9%, campesterol includes 0.05-4.95%, and sitostanol includes 0.05-4.95% of the sterols in the composition.

In another aspect, the disclosure features a composition including a plurality of lipid nanoparticles, wherein the plurality of lipid nanoparticles include an ionizable lipid and two or more sterols, wherein the two or more sterols include β-sitosterol, and sitostanol and β-sitosterol includes 95-99.9% of the sterols in the composition and sitostanol includes 0.1-5% of the sterols in the composition.

In some embodiments, the two or more sterols further includes campesterol. In some embodiments, β-sitosterol includes 95-99.9%, campesterol includes 0.05-4.95%, and sitostanol includes 0.05-4.95% of the sterols in the composition.

(ii) Non-Cationic Helper Lipids/Phospholipids

In some embodiments, the lipid-based composition (e.g., LNP) described herein comprises one or more non-cationic helper lipids. In some embodiments, the non-cationic helper lipid is a phospholipid. In some embodiments, the non-cationic helper lipid is a phospholipid substitute or replacement.

As used herein, the term “non-cationic helper lipid” refers to a lipid comprising at least one fatty acid chain of at least 8 carbons in length and at least one polar head group moiety. In one embodiment, the helper lipid is not a phosphatidyl choline (PC). In one embodiment the non-cationic helper lipid is a phospholipid or a phospholipid substitute. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-i77-glycero-3-phosphocholine (DSPC) substitute.

Phospholipids

The lipid composition of the pharmaceutical composition disclosed herein can comprise one or more non-cationic helper lipids. In some embodiments, the non-cationic helper lipids are phospholipids, for example, one or more saturated or (poly)unsaturated phospholipids or a combination thereof. In general, phospholipids comprise a phospholipid moiety and one or more fatty acid moieties. As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

The lipid component of a lipid nanoparticle of the disclosure may include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid may be a lipid according to Formula (H III):

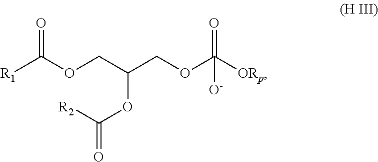

in which Rₚ represents a phospholipid moiety and R₁ and R₂ represent fatty acid moieties with or without unsaturation that may be the same or different. A phospholipid moiety may be selected from the non-limiting group consisting of phosphatidylcholine, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a LNP to facilitate membrane permeation or cellular recognition or in conjugating a LNP to a useful component such as a targeting or imaging moiety (e.g., a dye). Each possibility represents a separate embodiment of the present disclosure.

Phospholipids useful in the compositions and methods described herein may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine (18:3 (cis) PC), 1,2-diarachidonoyl-sn-glycero-3-phosphocholine (DAPC), 1,2-didocosahexaenoyl-sn-glycero-3-phosphocholine (22:6 (cis) PC) 1,2-diphytanoyl-sn-glycero-3-phosphoethanolamine (4ME 16.0 PE), 1,2-distearoyl-sn-glycero-3-phosphoethanolamine (DSPE), 1,2-dilinoleoyl-sn-glycero-3-phosphoethanolamine (PE(18:2/18:2), 1,2-dilinolenoyl-sn-glycero-3-phosphoethanolamine (PE 18:3(9Z, 12Z, 15Z), 1,2-diarachidonoyl-sn-glycero-3-phosphoethanolamine (DAPE 18:3 (9Z, 12Z, 15Z), 1,2-didocosahexaenoyl-sn-glycero-3-phosphoethanolamine (22:6 (cis) PE), 1,2-dioleoyl-sn-glycero-3-phospho-rac-(1-glycerol) sodium salt (DOPG),

and sphingomyelin. Each possibility represents a separate embodiment of the disclosure.

In some embodiments, a LNP includes DSPC. In certain embodiments, a LNP includes DOPE. In some embodiments, a LNP includes DMPE. In some embodiments, a LNP includes both DSPC and DOPE.

In one embodiment, a non-cationic helper lipid for use in an immune cell delivery LNP is selected from the group consisting of: DSPC, DMPE, and DOPC or combinations thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

Examples of phospholipids include, but are not limited to, the following:

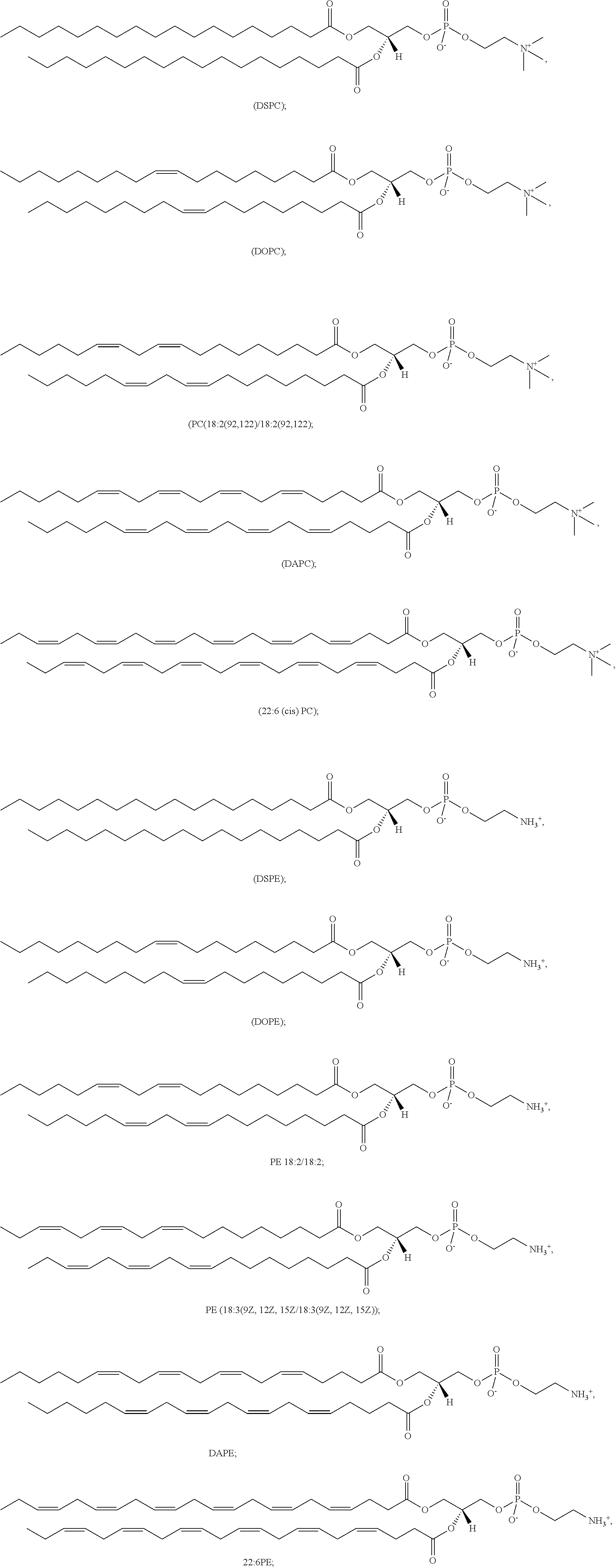

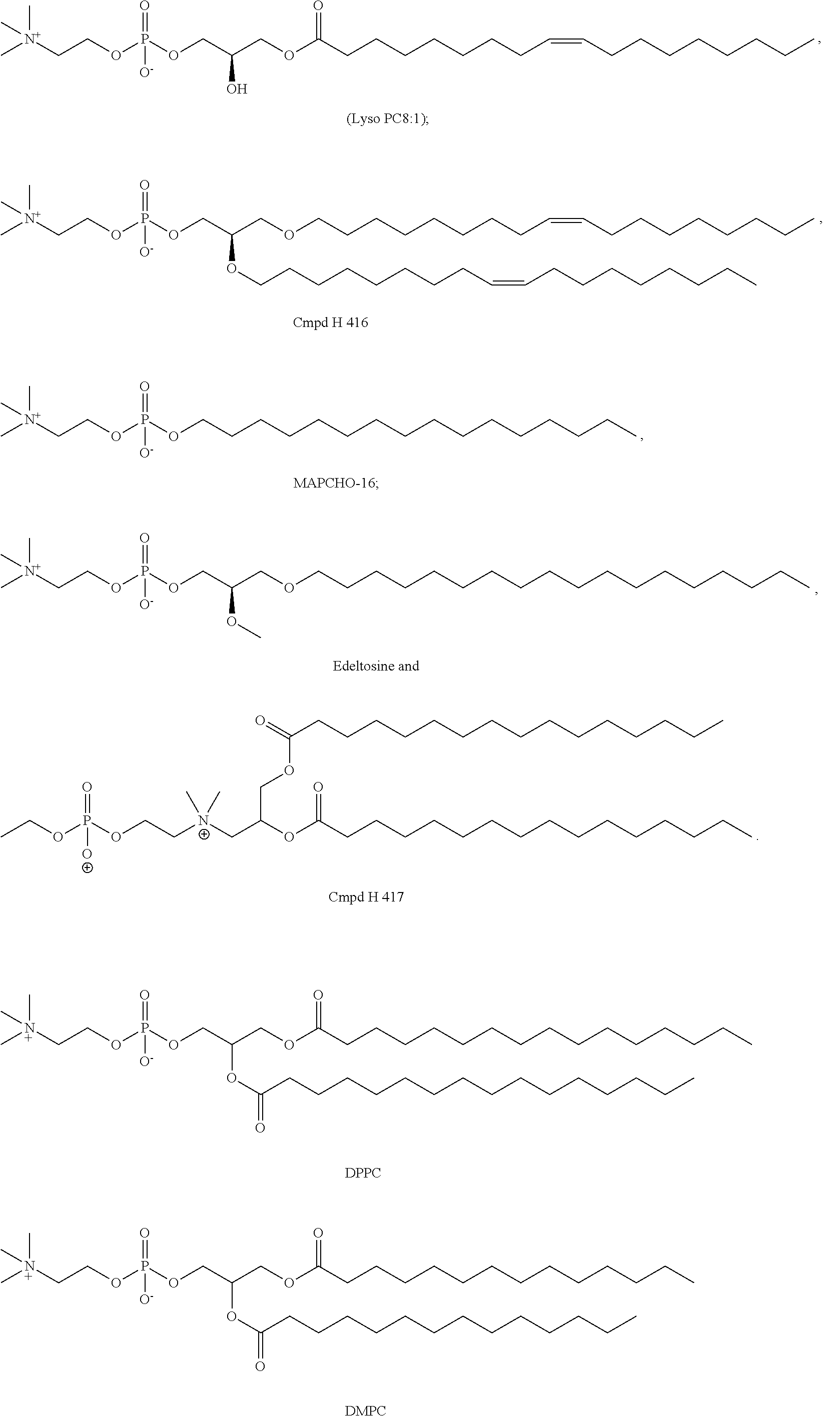

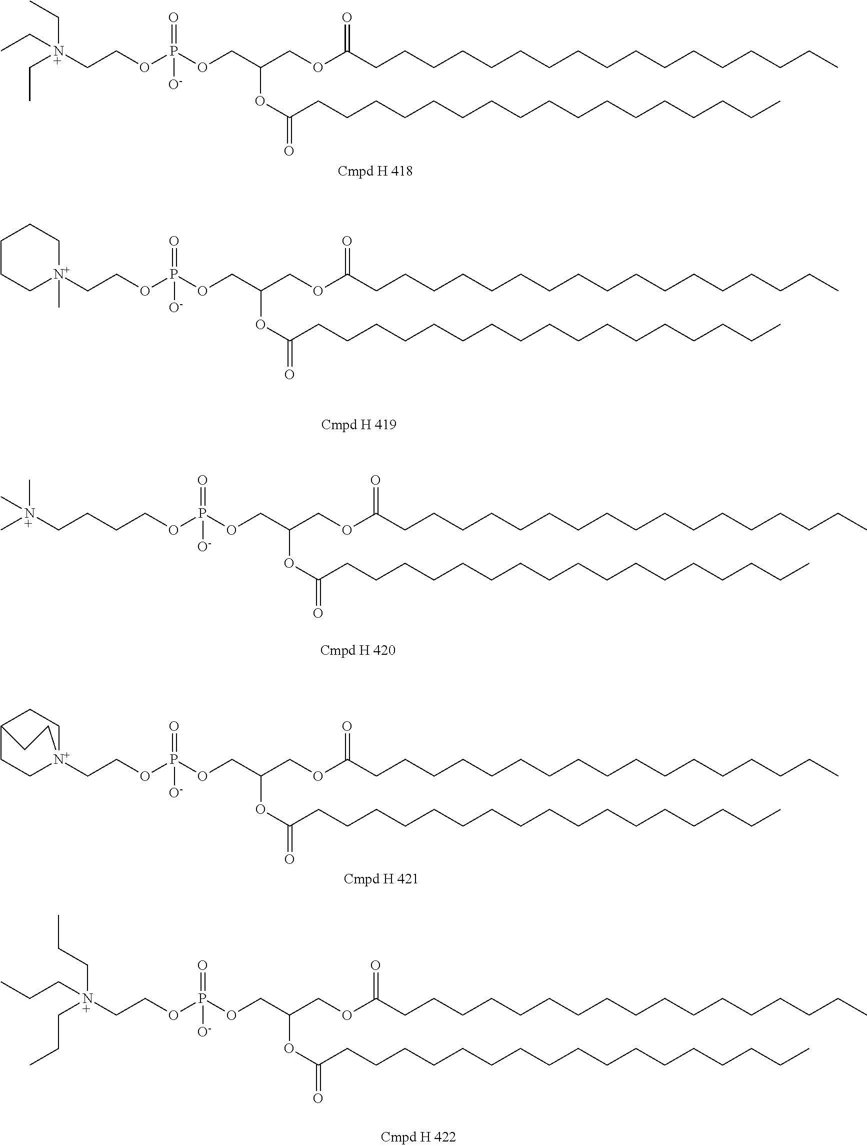

In certain embodiments, a phospholipid useful or potentially useful in the present disclosure is an analog or variant of DSPC (1,2-dioctadecanoyl-sn-glycero-3-phosphocholine). In certain embodiments, a phospholipid useful or potentially useful in the present disclosure is a compound of Formula (H IX):

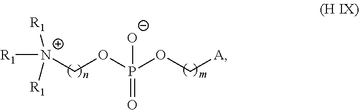

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

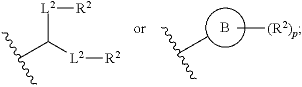

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

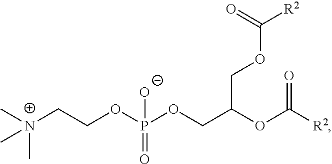

wherein each instance of R² is independently unsubstituted alkyl, unsubstituted alkenyl, or unsubstituted alkynyl.

i) Phospholipid Head Modifications

In certain embodiments, a phospholipid useful or potentially useful in the present disclosure comprises a modified phospholipid head (e.g., a modified choline group). In certain embodiments, a phospholipid with a modified head is DSPC, or analog thereof, with a modified quaternary amine. For example, in embodiments of Formula (IX), at least one of R¹ is not methyl. In certain embodiments, at least one of R¹ is not hydrogen or methyl. In certain embodiments, the compound of Formula (IX) is of one of the following formulae:

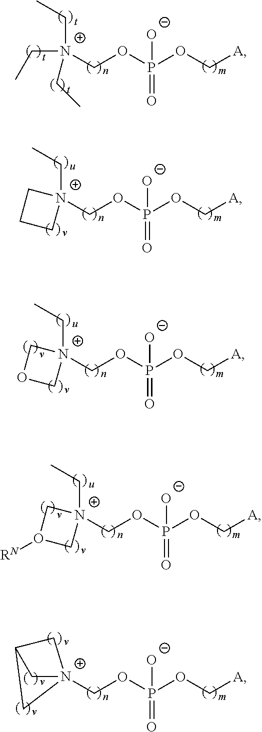

or a salt thereof, wherein:

each t is independently 1, 2, 3, 4, 5, 6, 7, 8, 9, or 10;

each u is independently 0, 1, 2, 3, 4, 5, 6, 7, 8, 9, or 10; and

each v is independently 1, 2, or 3.

In certain embodiments, the compound of Formula (H IX) is of one of the following formulae:

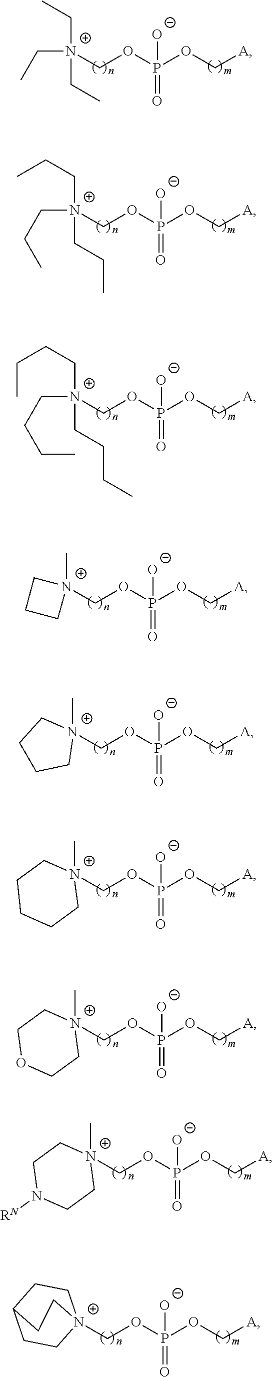

or a salt thereof.

In certain embodiments, a compound of Formula (H IX) is one of the following:

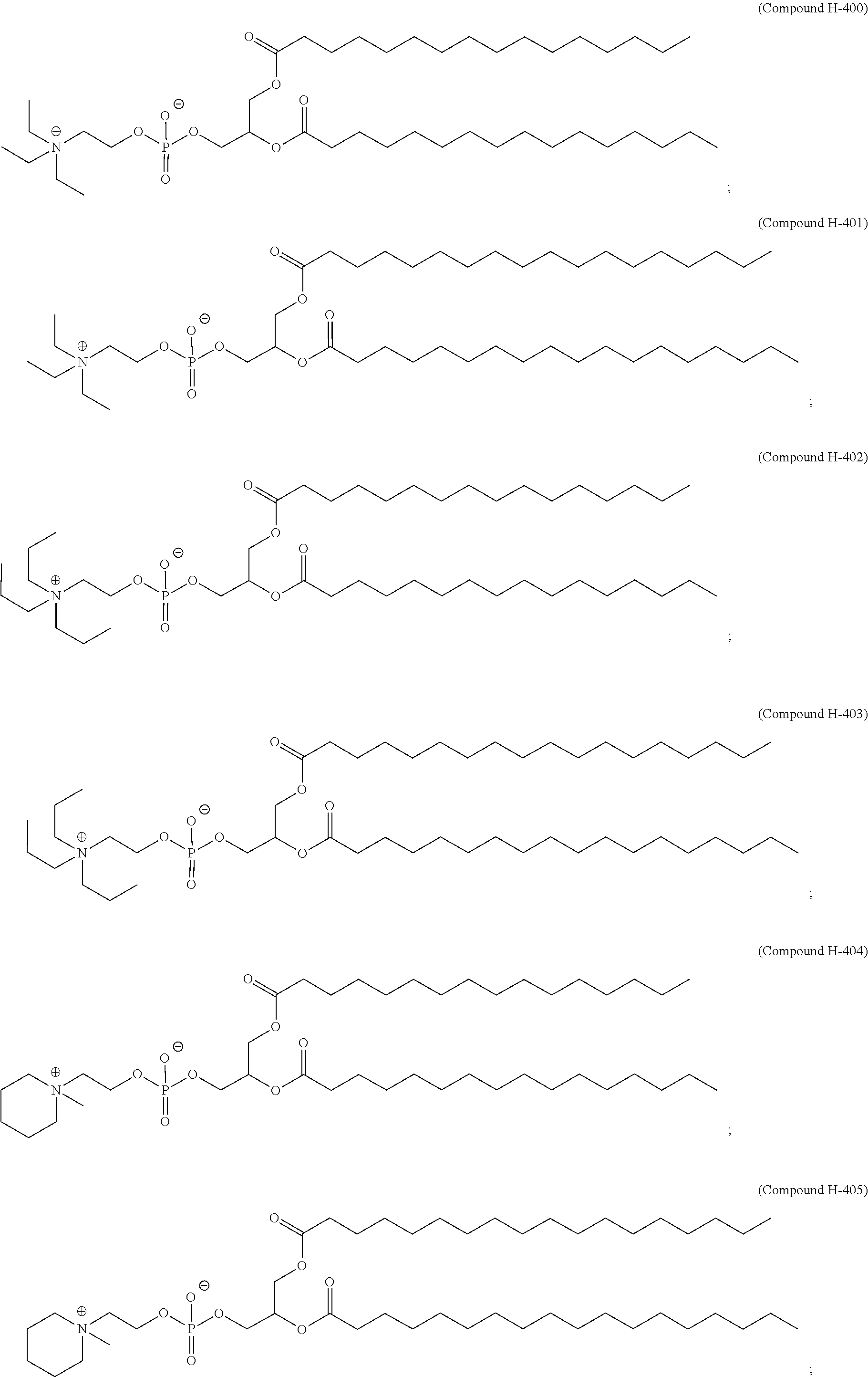

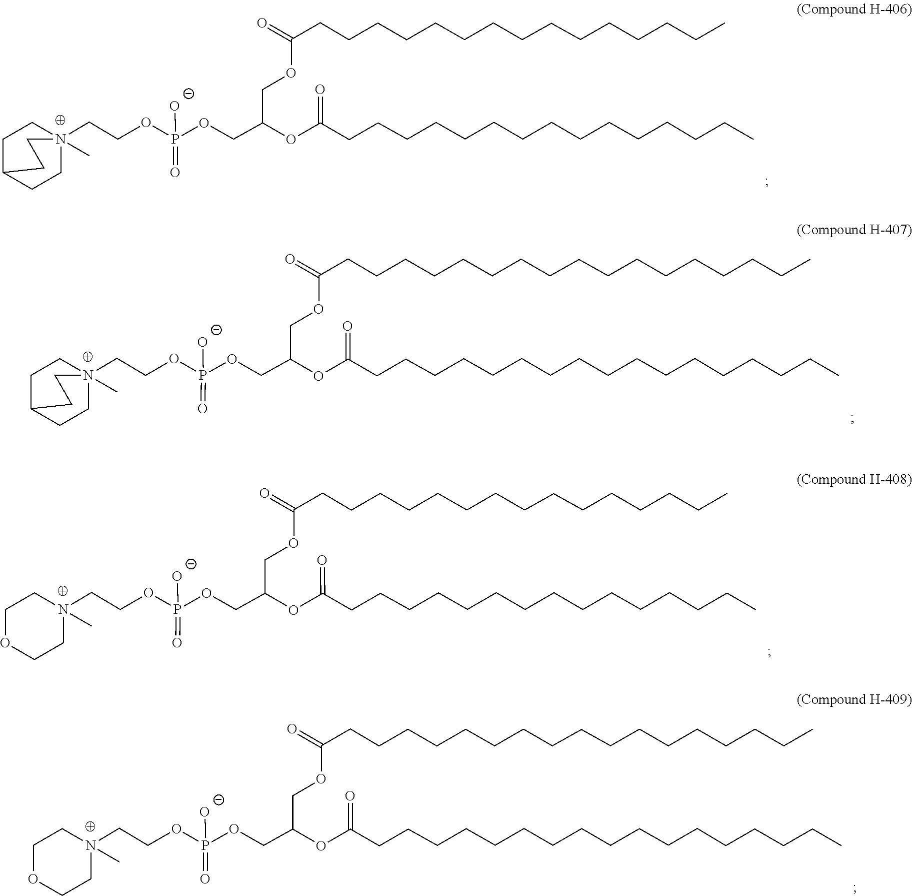

or a salt thereof.

In one embodiment, an immune cell delivery LNP comprises Compound H-409 as a non-cationic helper lipid.

### (II) Phospholipid Tail Modifications

In certain embodiments, a phospholipid useful or potentially useful in the present disclosure comprises a modified tail. In certain embodiments, a phospholipid useful or potentially useful in the present disclosure is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H IX) is of Formula (H IX-a), or a salt thereof, wherein at least one instance of R² is each instance of R² is optionally substituted C₁-30 alkyl, wherein one or more methylene units of R² are independently replaced with optionally substituted carbocyclylene, optionally substituted heterocyclylene, optionally substituted arylene, optionally substituted heteroarylene, N(R^{N}), 0, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), —NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), —OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O.

In certain embodiments, the compound of Formula (H IX) is of Formula (H IX-c):

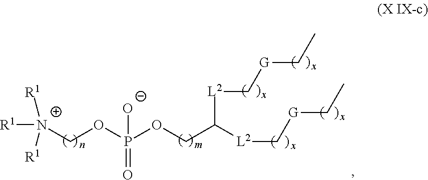

or a salt thereof, wherein:
each x is independently an integer between 0-30, inclusive; and

each instance is G is independently selected from the group consisting of optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), —OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O. Each possibility represents a separate embodiment of the present disclosure.

In certain embodiments, the compound of Formula (H IX-c) is of Formula (H IX-c-1):

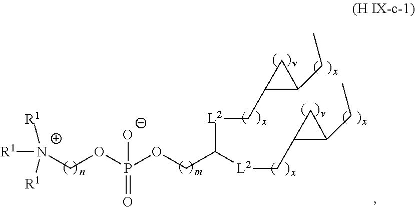

or salt thereof, wherein:
each instance of v is independently 1, 2, or 3.

In certain embodiments, the compound of Formula (H IX-c) is of Formula (H IX-c-2):

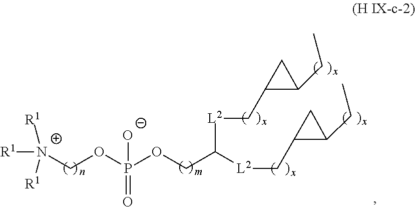

or a salt thereof.

In certain embodiments, the compound of Formula (IX-c) is of the following formula:

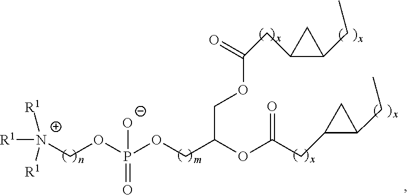

or a salt thereof.

In certain embodiments, the compound of Formula (H IX-c) is the following:

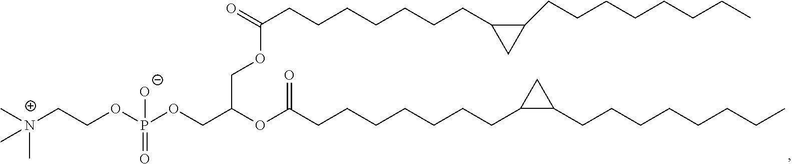 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H IX-c) is of Formula (H IX-c-3):

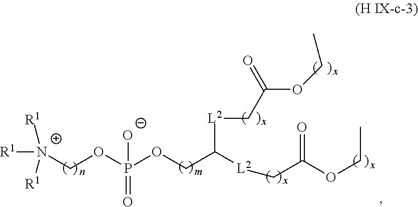

or a salt thereof.

In certain embodiments, the compound of Formula (H IX-c) is of the following formulae:

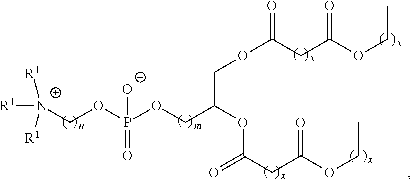

or a salt thereof.

In certain embodiments, the compound of Formula (H IX-c) is the following:

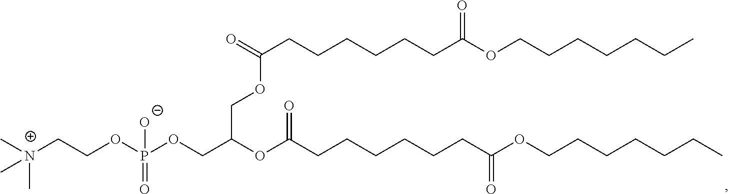 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present disclosure comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present disclosure is a compound of Formula (H IX), wherein n is 1, 3, 4, 5, 6, 7, 8, 9, or 10. For example, in certain embodiments, a compound of Formula (H IX) is of one of the following formulae:

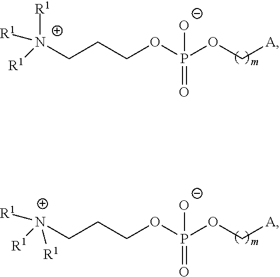

or a salt thereof.

In certain embodiments, a compound of Formula (H IX) is one of the following:

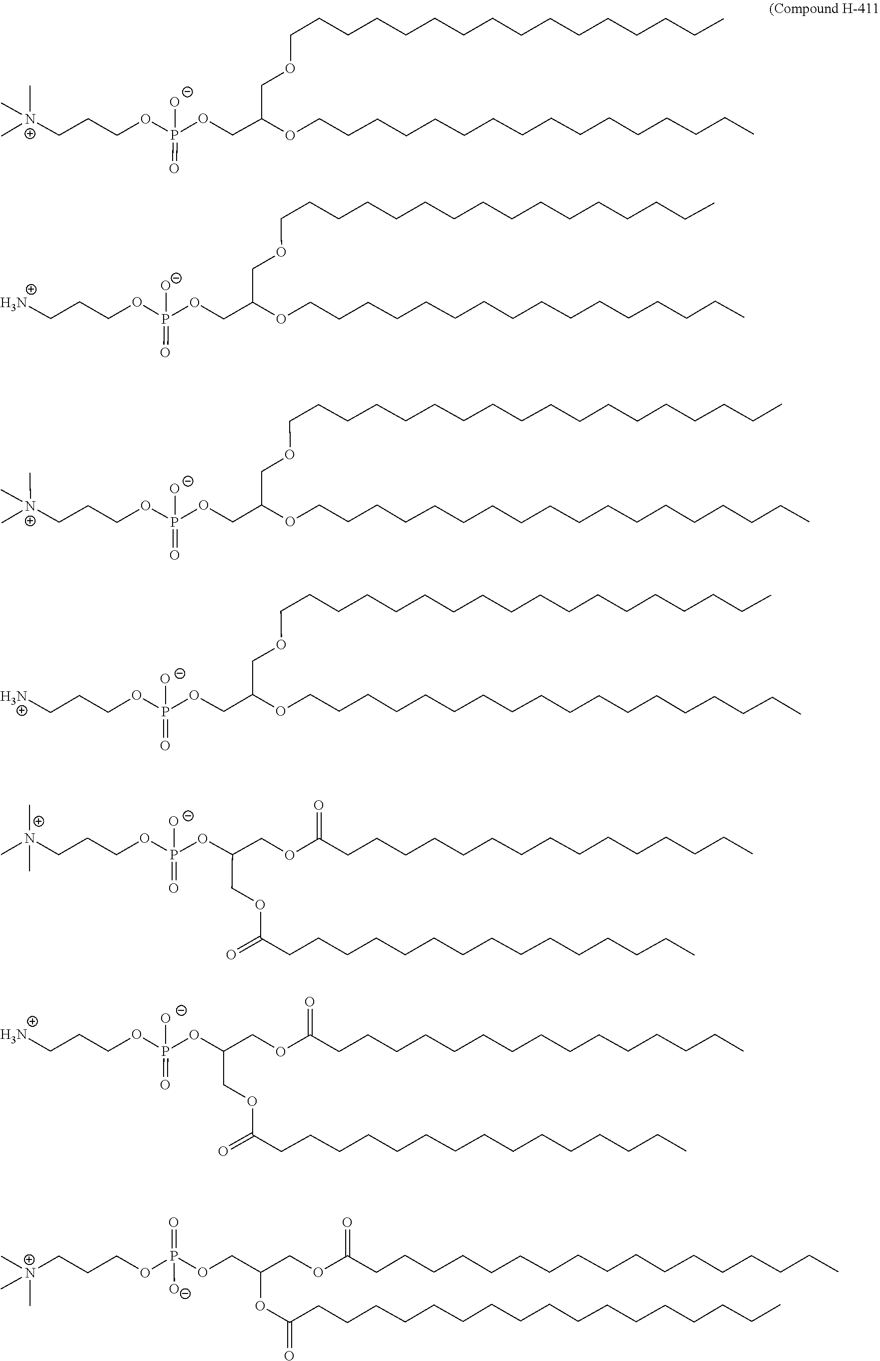

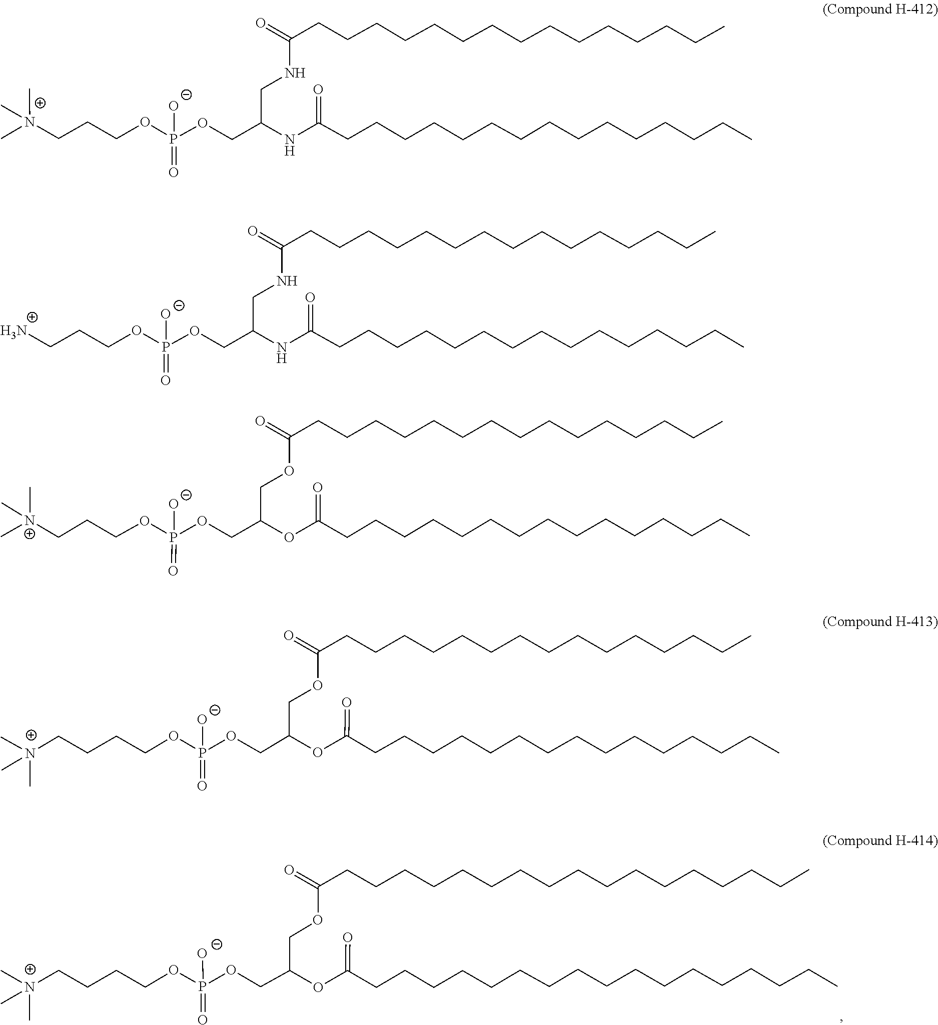

or salts thereof.

In certain embodiments, an alternative lipid is used in place of a phospholipid of the disclosure. Non-limiting examples of such alternative lipids include the following:

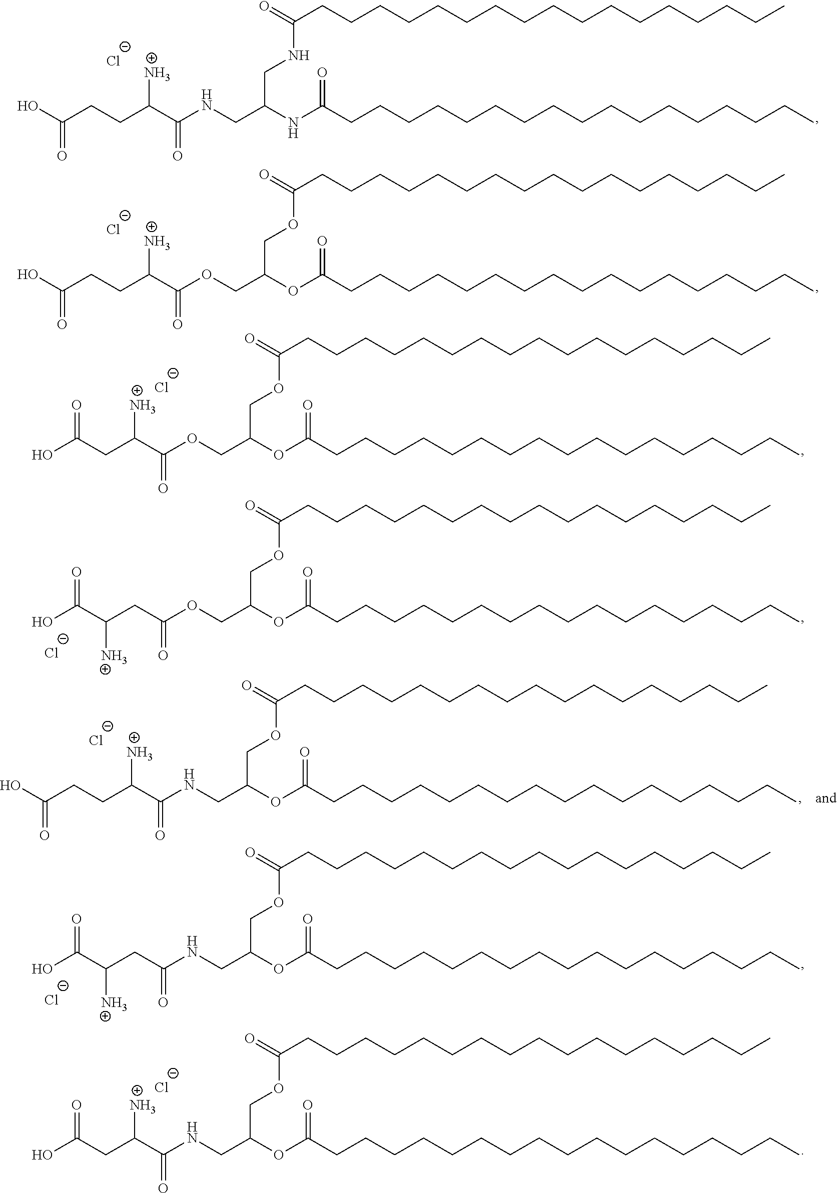

Phospholipid Tail Modifications

In certain embodiments, a phospholipid useful in the present disclosure comprises a modified tail. In certain embodiments, a phospholipid useful in the present disclosure is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H I) is of Formula (H I-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N}), O, S, C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H I-a) is of Formula (H I-c):

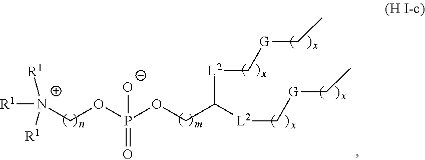

or a salt thereof, wherein:

each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present disclosure.

In certain embodiments, the compound of Formula (H I-c) is of Formula (H I-c-1):

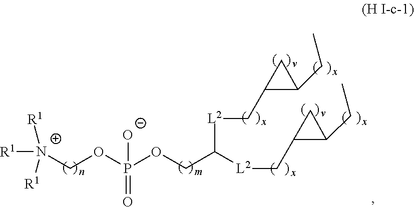

or salt thereof, wherein:

each instance of v is independently 1, 2, or 3.

In certain embodiments, the compound of Formula (H I-c) is of Formula (H I-c-2):

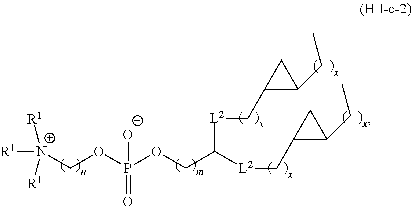

or a salt thereof.

In certain embodiments, the compound of Formula (I-c) is of the following formula:

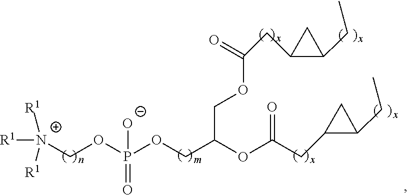

or a salt thereof.

In certain embodiments, the compound of Formula (H I-c) is the following:

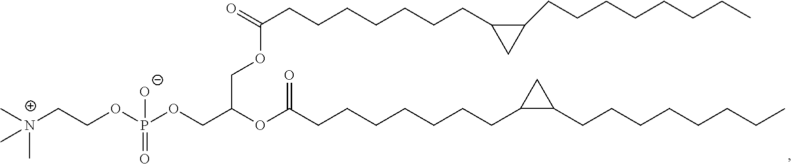 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H I-c) is of Formula (H I-c-3):

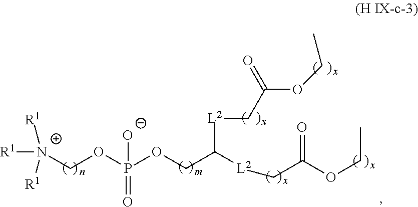

or a salt thereof.

In certain embodiments, the compound of Formula (H I-c) is of the following formulae:

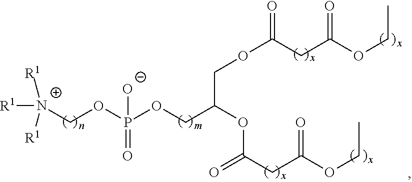

or a salt thereof.

In certain embodiments, the compound of Formula (H I-c) is the following:

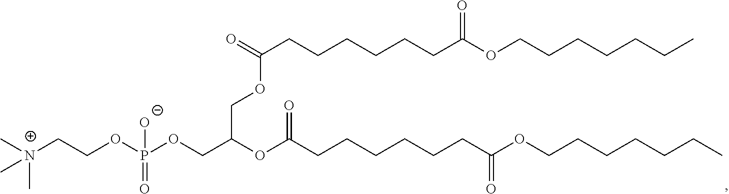 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

Phosphocholine Linker Modifications

In certain embodiments, a phospholipid useful in the present disclosure comprises a modified phosphocholine moiety, wherein the alkyl chain linking the quaternary amine to the phosphoryl group is not ethylene (e.g., n is not 2). Therefore, in certain embodiments, a phospholipid useful in the present disclosure is a compound of Formula (H I), wherein n is 1, 3, 4, 5, 6, 7, 8, 9, or 10. For example, in certain embodiments, a compound of Formula (H I) is of one of the following formulae:

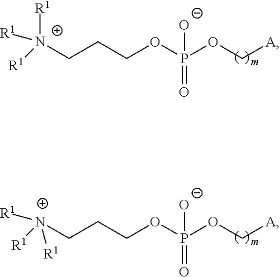

or a salt thereof.

In certain embodiments, a compound of Formula (H I) is one of the following:

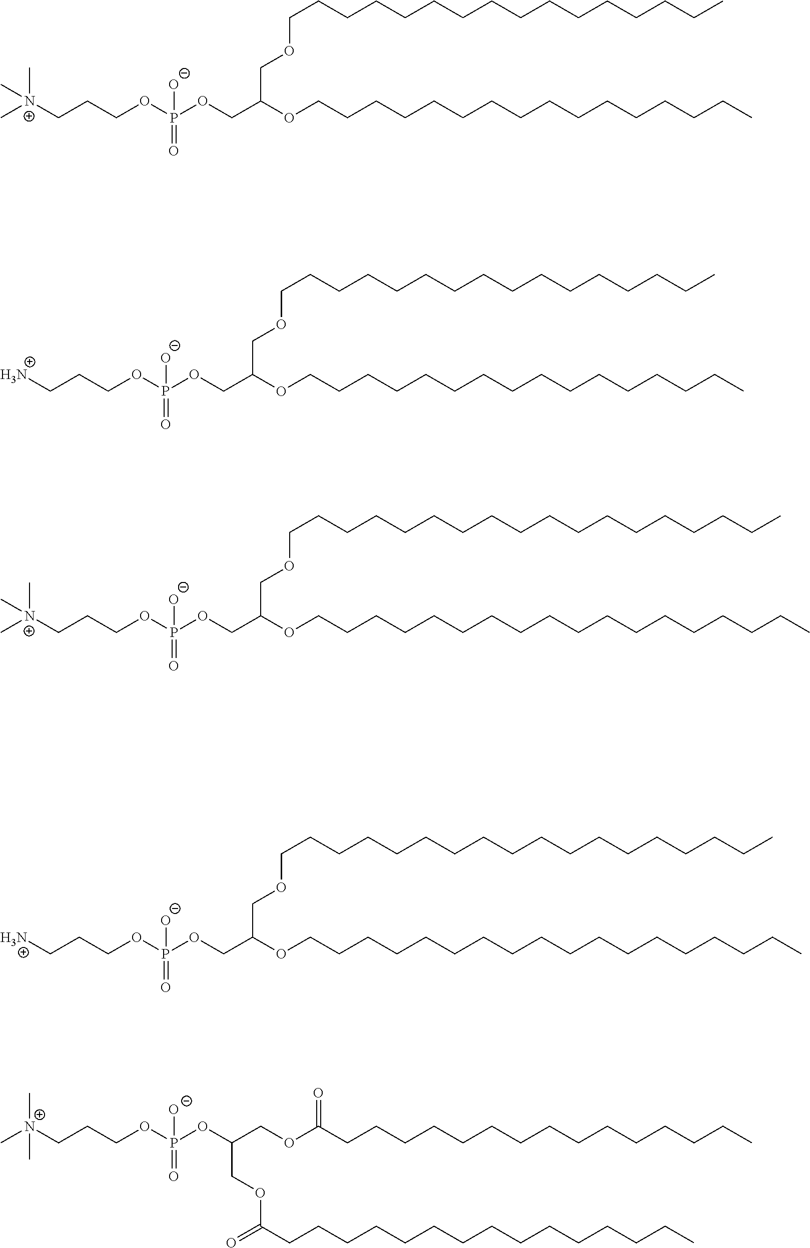

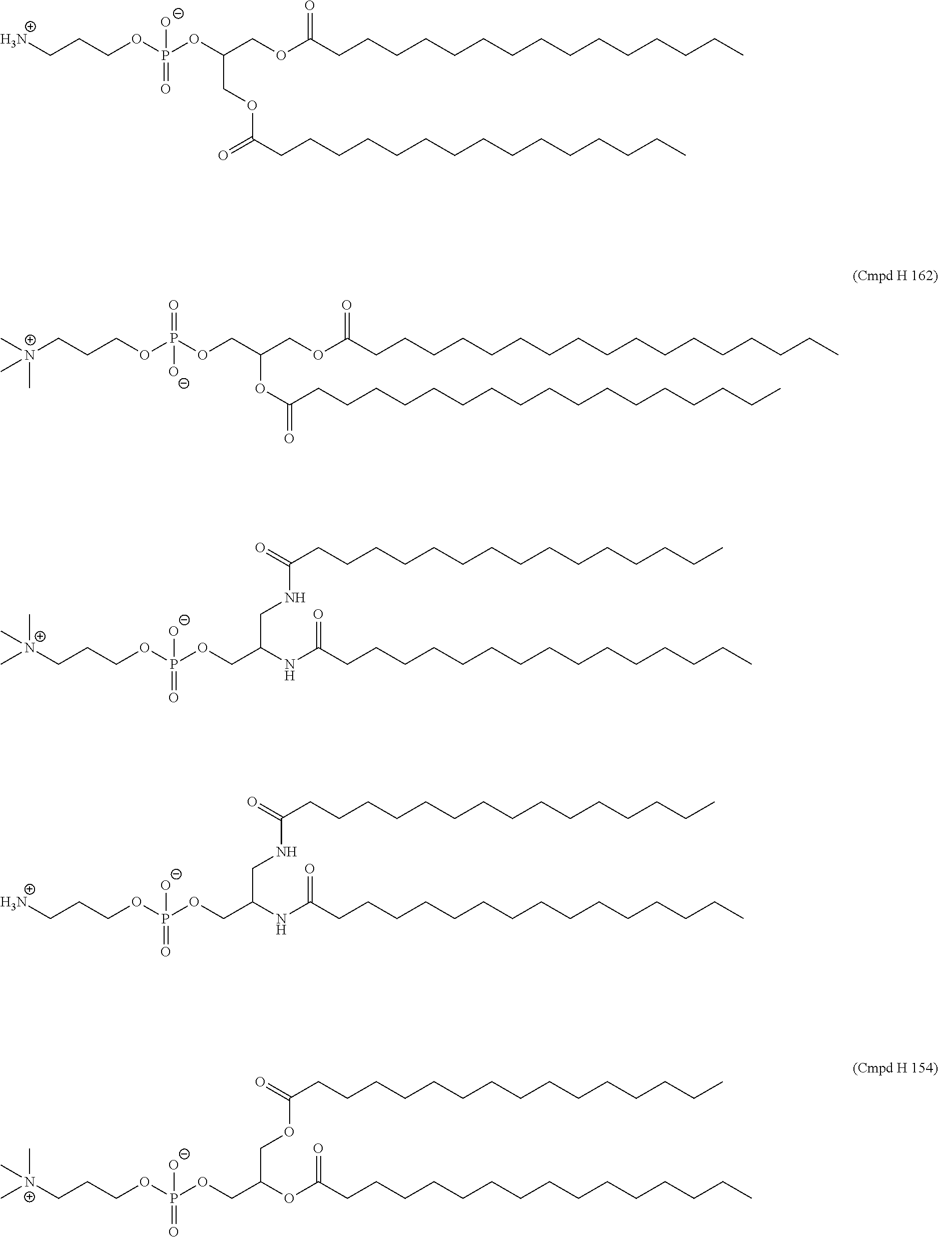

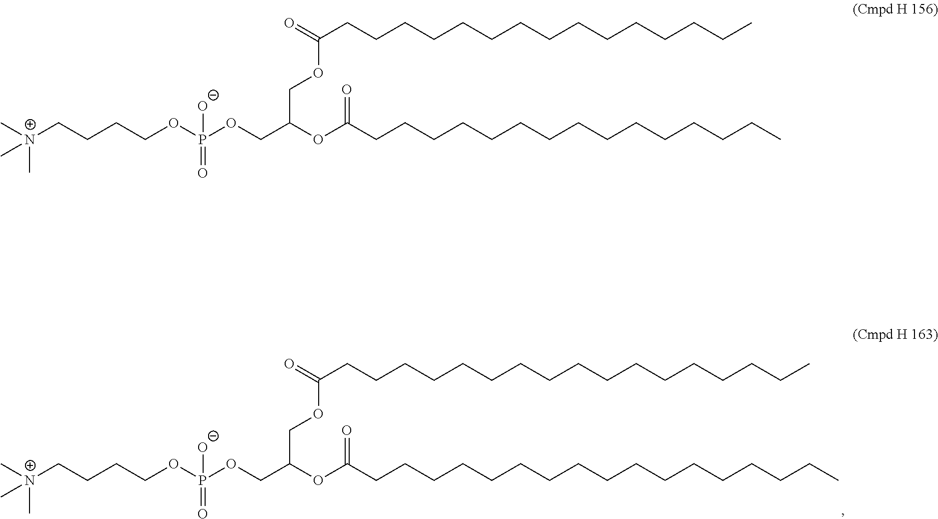

or salts thereof.

Numerous LNP formulations having phospholipids other than DSPC were prepared and tested for activity, as demonstrated in the examples below.

### Phospholipid Substitute or Replacement

In some embodiments, the lipid-based composition (e.g., lipid nanoparticle) comprises an oleic acid or an oleic acid analog in place of a phospholipid. In some embodiments, an oleic acid analog comprises a modified oleic acid tail, a modified carboxylic acid moiety, or both. In some embodiments, an oleic acid analog is a compound wherein the carboxylic acid moiety of oleic acid is replaced by a different group.

In some embodiments, the lipid-based composition (e.g., lipid nanoparticle) comprises a different zwitterionic group in place of a phospholipid.

Exemplary phospholipid substitutes and/or replacements are provided in Published PCT Application WO 2017/099823, herein incorporated by reference.

Exemplary phospholipid substitutes and/or replacements are provided in Published PCT Application WO 2017/099823, herein incorporated by reference.

(iii) PEG Lipids

Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DS G), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

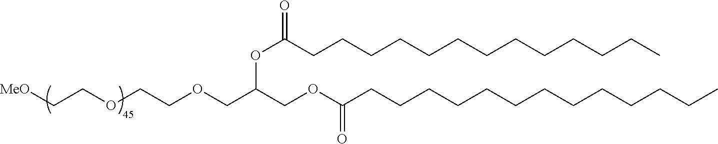

In one embodiment, PEG lipids useful in the present disclosure can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present disclosure.

In some embodiments, the PEG lipid is a compound of Formula (PI):

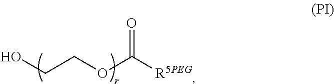

or a salt or isomer thereof, wherein:

r is an integer between 1 and 100;

R^{5PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group.

For example, R^{5PEG} is C₁₇ alkyl. For example, the PEG lipid is a compound of Formula (PI-a):

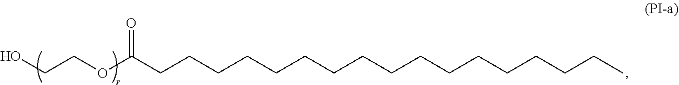

or a salt or isomer thereof, wherein r is an integer between 1 and 100.

For example, the PEG lipid is a compound of the following formula:

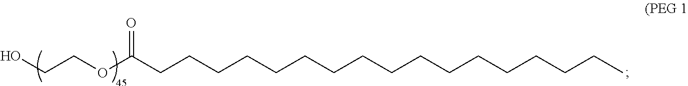

also referred to as Compound 428 below), or a salt or isomer thereof.

The PEG lipid may be a compound of Formula (PII):

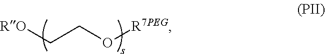

or a salt or isomer thereof, wherein:

s is an integer between 1 and 100;

R″ is a hydrogen, C₁₋₁₀ alkyl, or an oxygen protecting group; R^{7PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group.

In some embodiments, R^{7PEG} is C₁₀₋₆₀ alkyl, and one or more of the methylene groups of R^{7PEG} are replaced with —C(O)—. For example, R^{7PEG} is C₃₁ alkyl, and two of the methylene groups of R^{7PEG} are replaced with —C(O)—.

In some embodiments, R″ is methyl.

In some embodiments, the PEG lipid is a compound of Formula (PII-a):

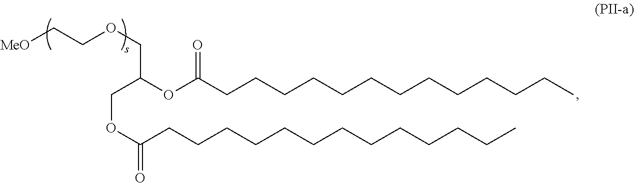

or a salt or isomer thereof, wherein s is an integer between 1 and 100.

For example, the PEG lipid is a compound of the following formula:

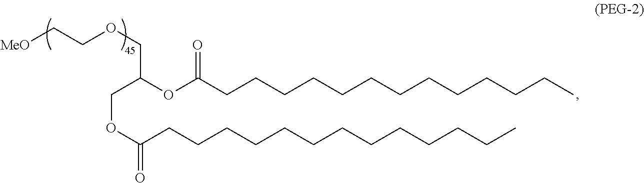

or a salt or isomer thereof.

In certain embodiments, a PEG lipid useful in the present disclosure is a compound of Formula (PIII). Provided herein are compounds of Formula (PIII):

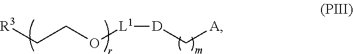

or salts thereof, wherein:

R³ is —OR^{o};

R^{o} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, 0, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

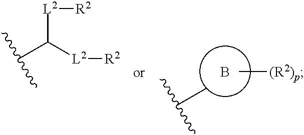

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), 0, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (PIII) is a PEG-OH lipid (i.e., R³ is —OR^{O}, and R^{O} is hydrogen). In certain embodiments, the compound of Formula (PIII) is of Formula (PIII-OH):

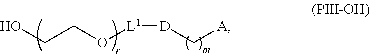

or a salt thereof.

In certain embodiments, D is a moiety obtained by click chemistry (e.g., triazole). In certain embodiments, the compound of Formula (PIII) is of Formula (PIII-a-1) or (PIII-a-2):

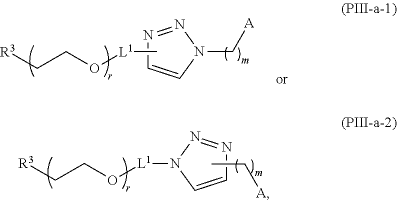

or a salt thereof.

In certain embodiments, the compound of Formula (PIII) is of one of the following formulae:

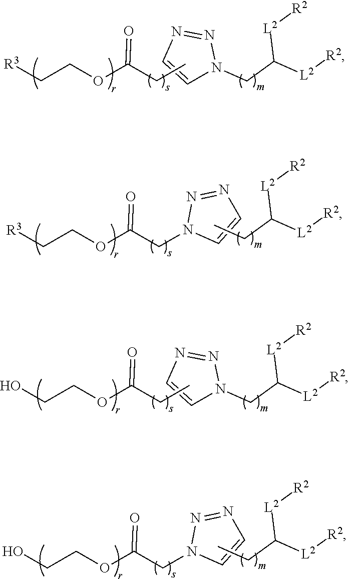

or a salt thereof, wherein

s is 0, 1, 2, 3, 4, 5, 6, 7, 8, 9, or 10.

In certain embodiments, the compound of Formula (PIII) is of one of the following formulae:

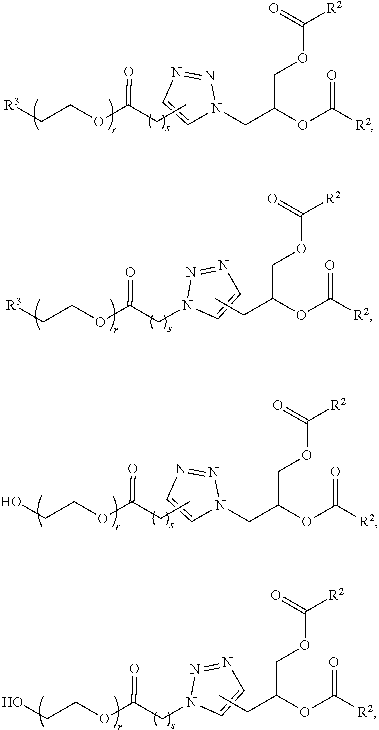

or a salt thereof.

In certain embodiments, a compound of Formula (PIII) is of one of the following formulae:

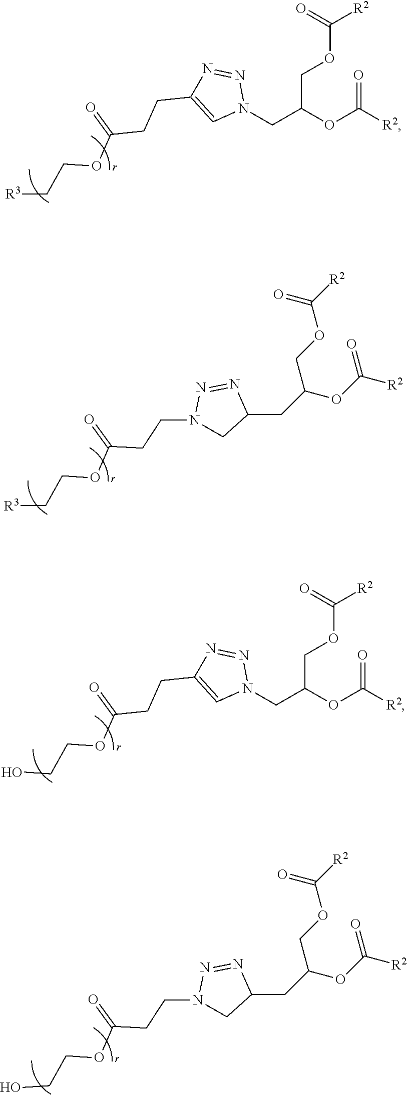

or a salt thereof.

In certain embodiments, a compound of Formula (PIII) is of one of the following formulae:

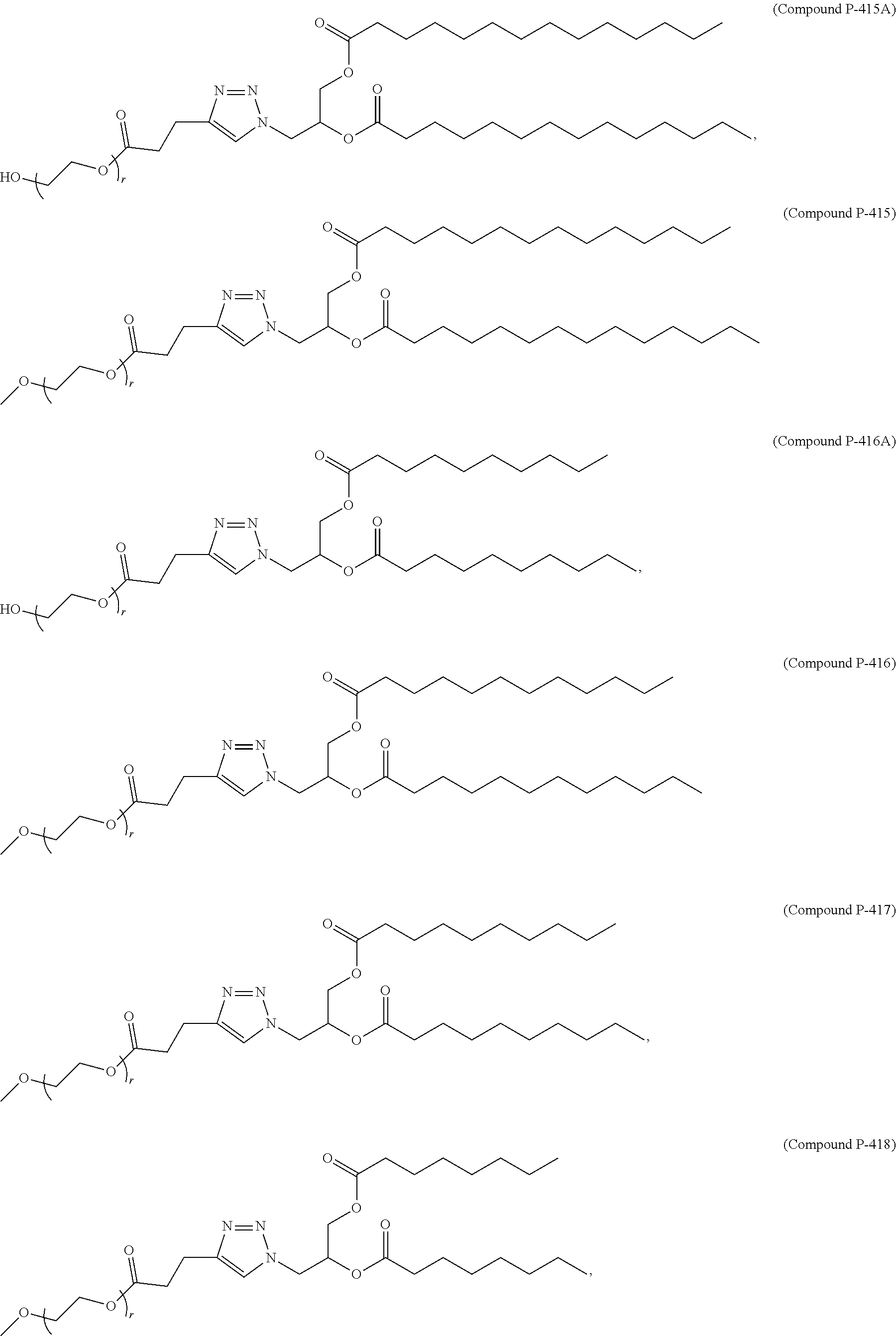

or a salt thereof.

In certain embodiments, D is a moiety cleavable under physiological conditions (e.g., ester, amide, carbonate, carbamate, urea). In certain embodiments, a compound of Formula (PIII) is of Formula (PIII-b-1) or (PIII-b-2):

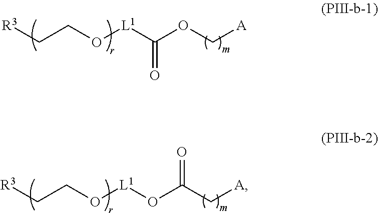

or a salt thereof.

In certain embodiments, a compound of Formula (PIII) is of Formula (PIII-b-1-OH) or (PIII-b-2-OH):

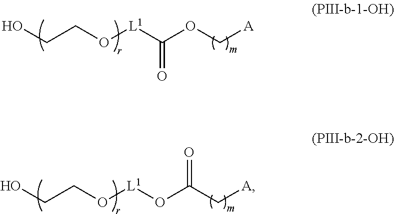

or a salt thereof.

In certain embodiments, the compound of Formula (PIII) is of one of the following formulae:

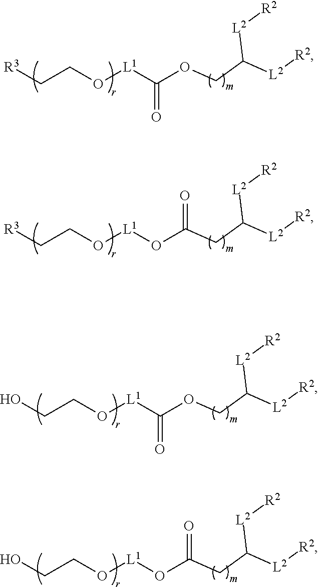

or a salt thereof.

In certain embodiments, a compound of Formula (PIII) is of one of the following formulae:

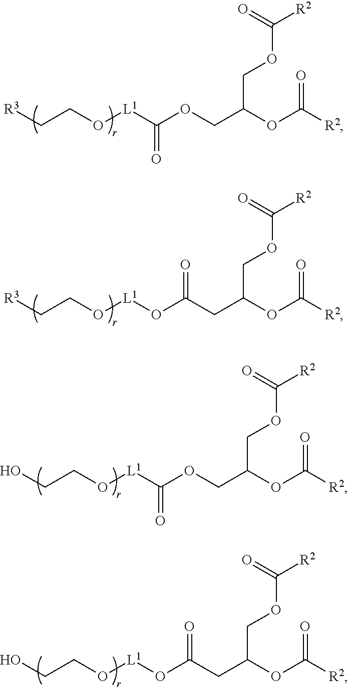

or a salt thereof.

In certain embodiments, a compound of Formula (PIII) is of one of the following formulae:

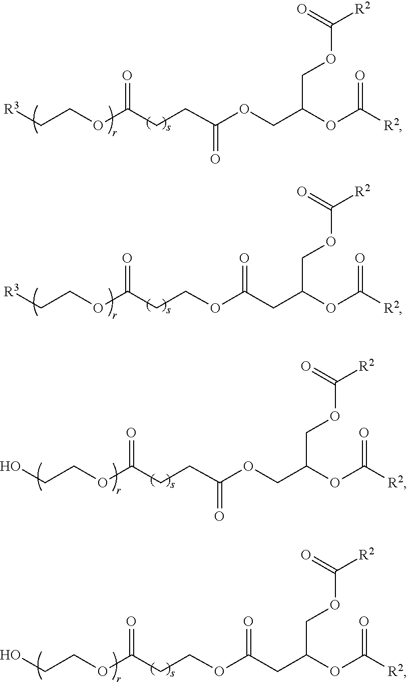

or a salt thereof.

In certain embodiments, a compound of Formula (PIII) is of one of the following formulae:

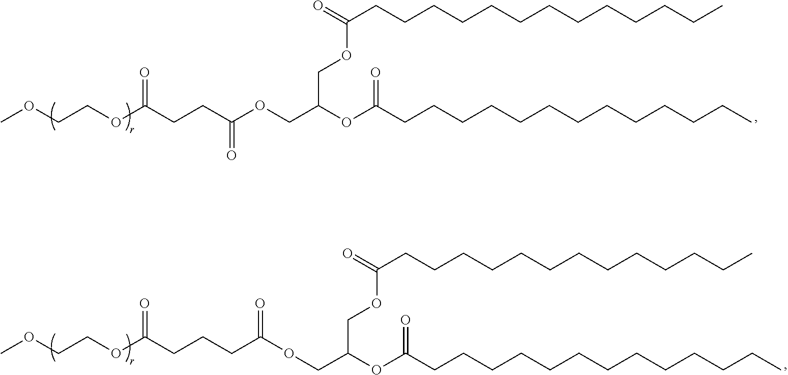

or salts thereof.

In certain embodiments, a PEG lipid useful in the present disclosure is a PEGylated fatty acid. In certain embodiments, a PEG lipid useful in the present disclosure is a compound of Formula (PIV). Provided herein are compounds of Formula (PIV):

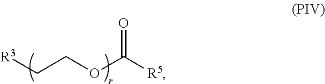

or a salts thereof, wherein:

R³ is —OR^{O};

R^{O} is hydrogen, optionally substituted alkyl or an oxygen protecting group;
r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O), OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (PIV is of Formula (PIV-OH):

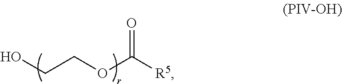

or a salt thereof. In some embodiments, r is 40-50. In some embodiments, r is 45.

In certain embodiments, a compound of Formula (PIV) is of one of the following formulae:

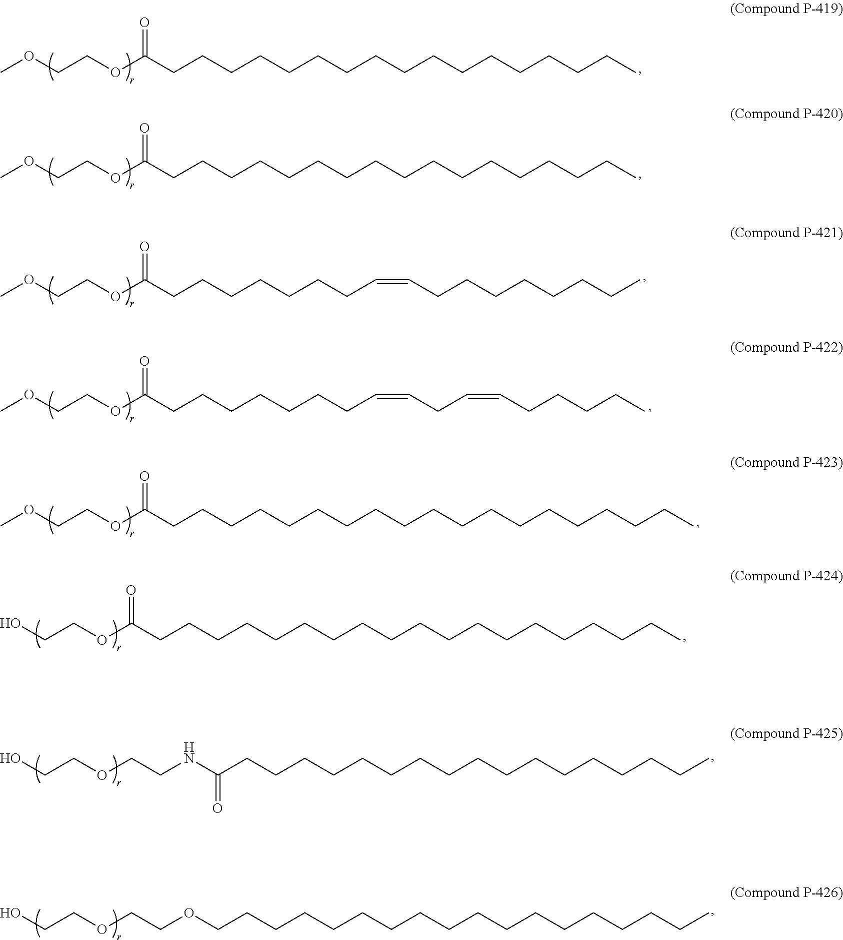

or a salt thereof. In some embodiments, r is 40-50. In some embodiments, r is 45.

In yet other embodiments the compound of Formula (PIV) is:

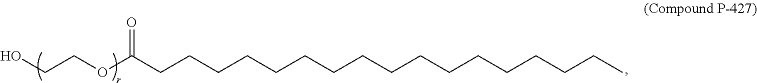

or a salt thereof.

In one embodiment, the compound of Formula (PIV) is

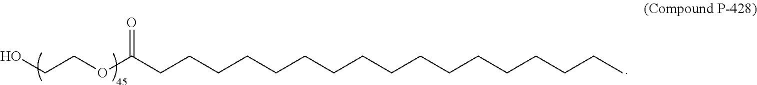

In one aspect, provided herein are lipid nanoparticles (LNPs) comprising PEG lipids of Formula (PV):

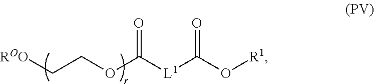

or pharmaceutically acceptable salts thereof; wherein:

L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;

R¹ is optionally substituted C₅₋₃₀ alkyl, optionally substituted C₅₋₃₀ alkenyl, or optionally substituted C₅₋₃₀ alkynyl;

R^{O} is hydrogen, optionally substituted alkyl, optionally substituted acyl, or an oxygen protecting group; and r is an integer from 2 to 100, inclusive.

In certain embodiments, the PEG lipid of Formula (PV) is of the following formula:

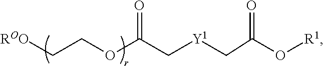

or a pharmaceutically acceptable salt thereof; wherein:

Y¹ is a bond, —CR₂—, —O—, —NR^{N}—, or —S—;

each instance of R is independently hydrogen, halogen, or optionally substituted alkyl; and

R^{N} is hydrogen, optionally substituted alkyl, optionally substituted acyl, or a nitrogen protecting group.

In certain embodiments, the PEG lipid of Formula (PV) is of one of the following formulae:

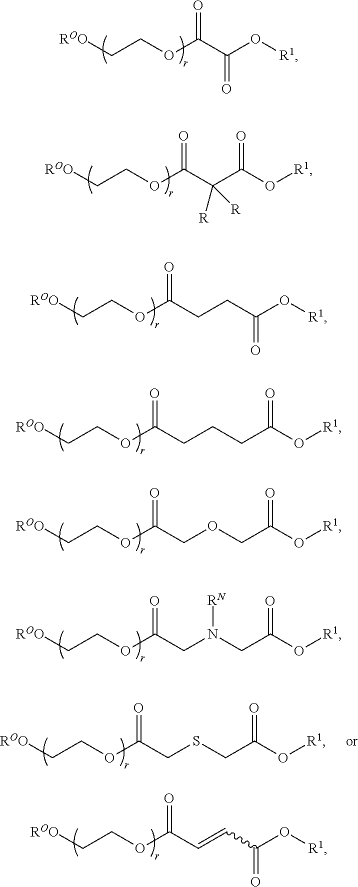

or a pharmaceutically acceptable salt thereof, wherein:

each instance of R is independently hydrogen, halogen, or optionally substituted alkyl.

In certain embodiments, the PEG lipid of Formula (PV) is of one of the following formulae:

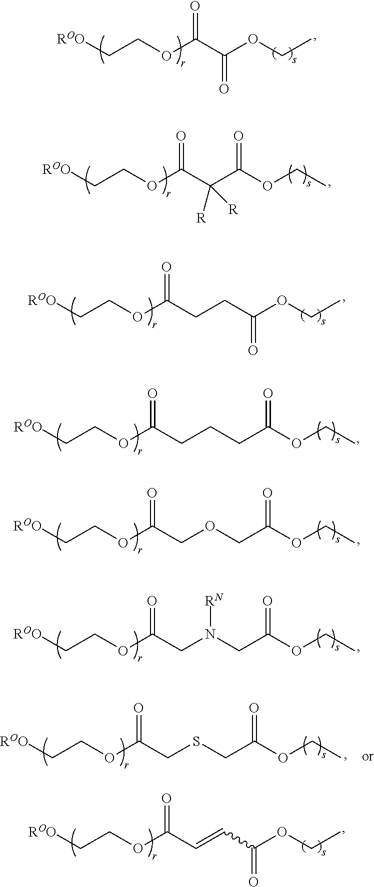

or a pharmaceutically acceptable salt thereof; wherein:

s is an integer from 5-25, inclusive.

In certain embodiments, the PEG lipid of Formula (PV) is of one of the following formulae:

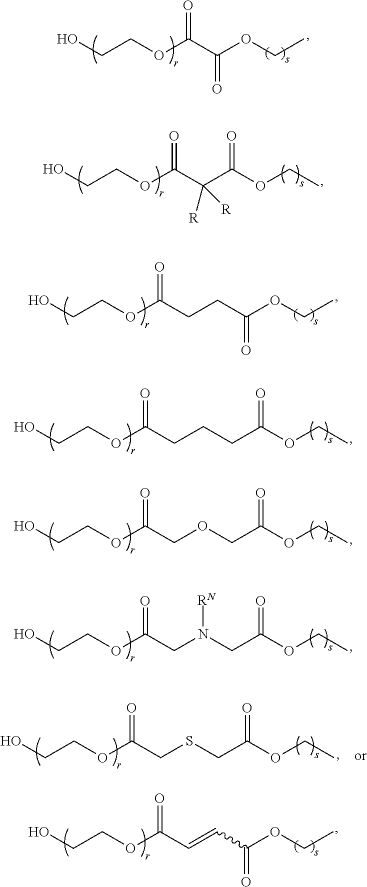

or a pharmaceutically acceptable salt thereof.

In certain embodiments, the PEG lipid of Formula (PV) is selected from the group consisting of:

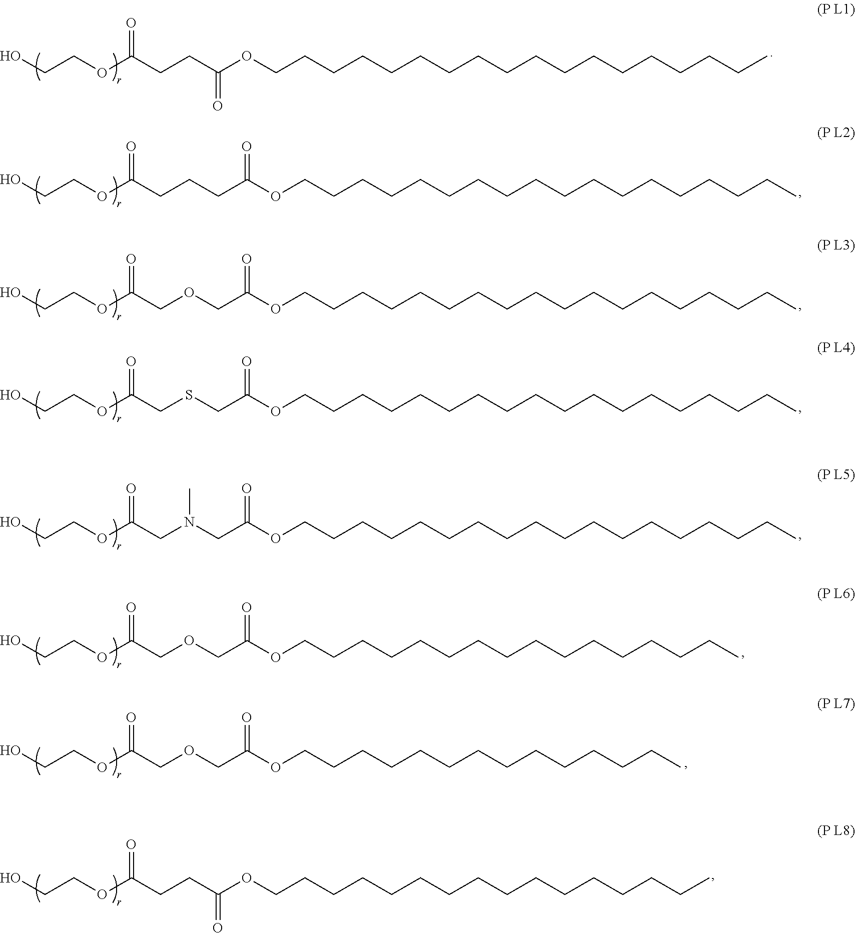

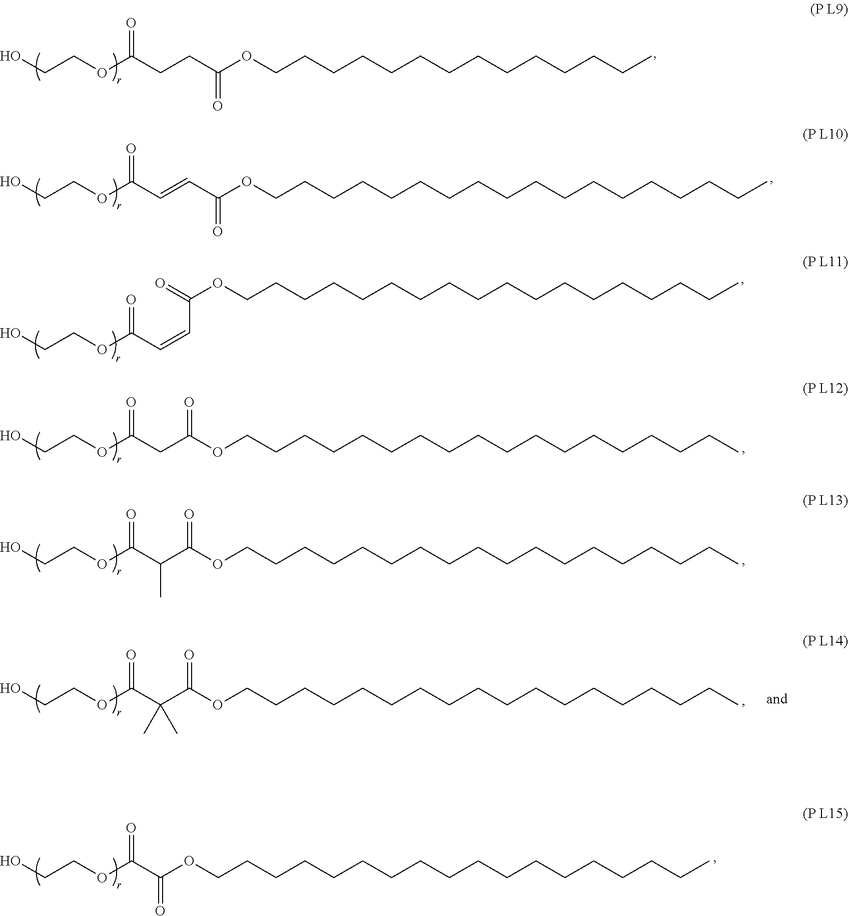

and pharmaceutically acceptable salts thereof.

In another aspect, provided herein are lipid nanoparticles (LNPs) comprising PEG lipids of Formula (PVI):

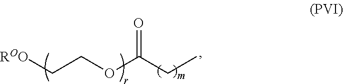

or pharmaceutically acceptable salts thereof; wherein:

R^{O} is hydrogen, optionally substituted alkyl, optionally substituted acyl, or an oxygen protecting group;

r is an integer from 2 to 100, inclusive; and

m is an integer from 5-15, inclusive, or an integer from 19-30, inclusive.

In certain embodiments, the PEG lipid of Formula (PVI) is of one of the following formulae:

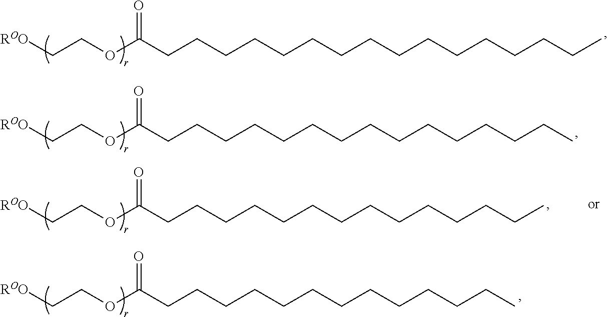

or a pharmaceutically acceptable salt thereof.

In certain embodiments, the PEG lipid of Formula (PVI) is of one of the following formulae:

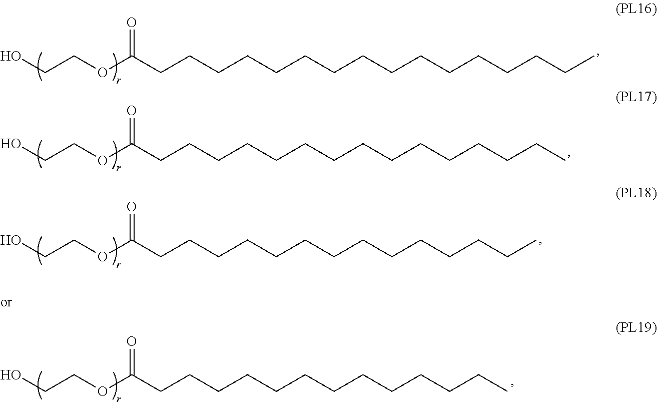

or a pharmaceutically acceptable salt thereof.

In another aspect, provided herein are lipid nanoparticles (LNPs) comprising PEG lipids of Formula (PVII):

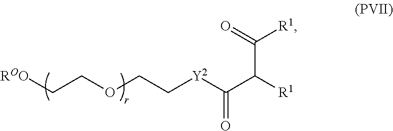

or pharmaceutically acceptable salts thereof, wherein:

Y² is —O—, —NR^{N}—, or —S—

each instance of R¹ is independently optionally substituted C₅₋₃₀ alkyl, optionally substituted C₅₋₃₀ alkenyl, or optionally substituted C₅₋₃₀ alkynyl;

R^{O} is hydrogen, optionally substituted alkyl, optionally substituted acyl, or an oxygen protecting group;

R^{N} is hydrogen, optionally substituted alkyl, optionally substituted acyl, or a nitrogen protecting group; and

r is an integer from 2 to 100, inclusive.

In certain embodiments, the PEG lipid of Formula (PVII) is of one of the following formulae:

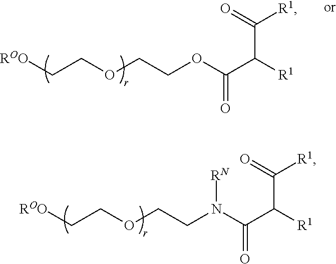

or a pharmaceutically acceptable salt thereof.

In certain embodiments, the PEG lipid of Formula (PVII) is of one of the following formulae:

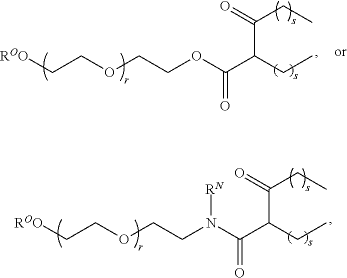

or a pharmaceutically acceptable salt thereof; wherein:

each instance of s is independently an integer from 5-25, inclusive.

In certain embodiments, the PEG lipid of Formula (PVII) is of one of the following formulae:

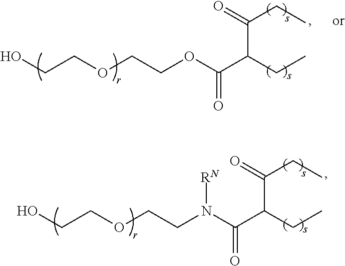

or a pharmaceutically acceptable salt thereof

In certain embodiments, the PEG lipid of Formula (PVII) is selected from the group consisting of:

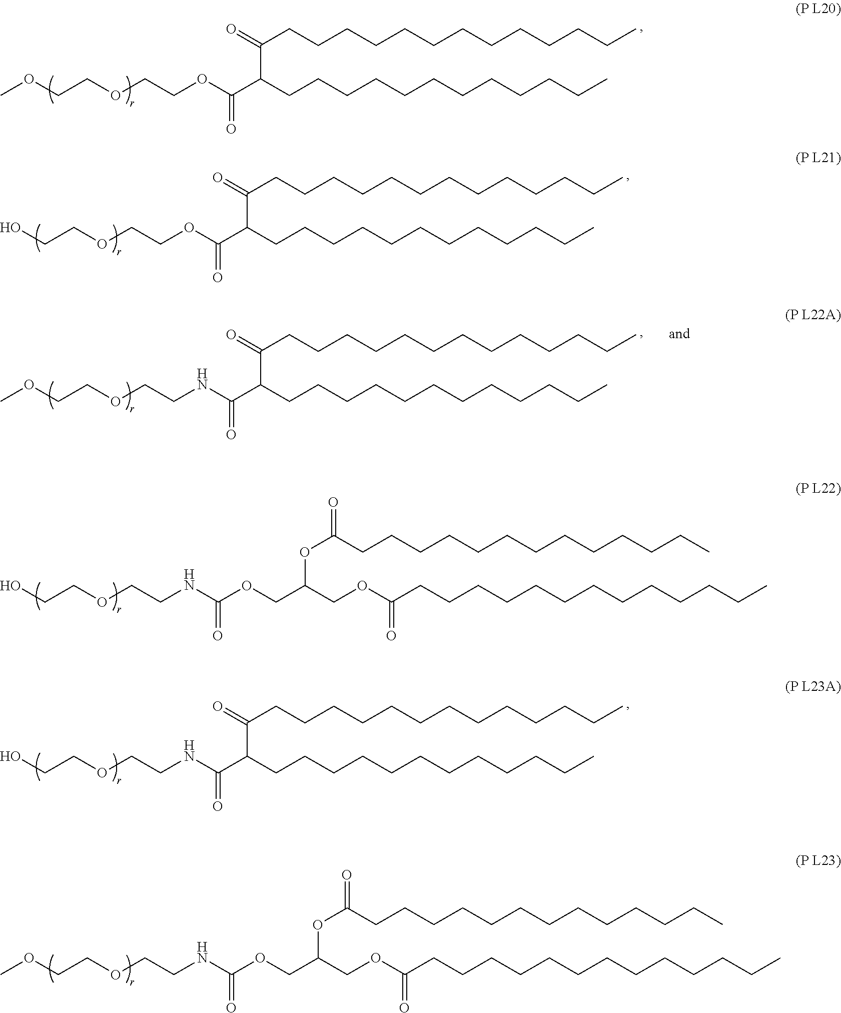

and pharmaceutically acceptable salts thereof.

In another aspect, provided herein are lipid nanoparticles (LNPs) comprising PEG lipids of Formula (PVIII):

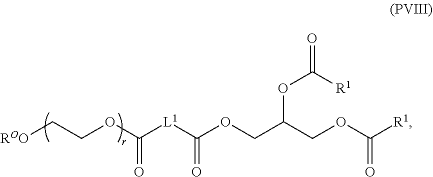

or pharmaceutically acceptable salts thereof, wherein:

L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;

each instance of R¹ is independently optionally substituted C₅₋₃₀ alkyl, optionally substituted C₃₋₃₀ alkenyl, or optionally substituted C₅₋₃₀ alkynyl;

R^{O} is hydrogen, optionally substituted alkyl, optionally substituted acyl, or an oxygen protecting group;

r is an integer from 2 to 100, inclusive;

provided that when L¹ is —CH₂CH₂— or —CH₂CH₂CH₂—, R^{O} is not methyl.

In certain embodiments, when L¹ is optionally substituted C₂ or C₃ alkylene, R^{O} is not optionally substituted alkyl. In certain embodiments, when L¹ is optionally substituted C₂ or C₃ alkylene, R^{O} is hydrogen. In certain embodiments, when L¹ is —CH₂CH₂— or —CH₂CH₂CH₂—, R^{o} is not optionally substituted alkyl. In certain embodiments, when L¹ is —CH₂CH₂— or —CH₂CH₂CH₂—, R^{O} is hydrogen.

In certain embodiments, the PEG lipid of Formula (PVIII) is of the formula:

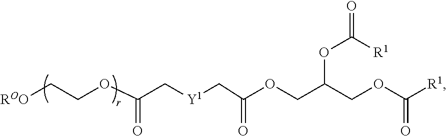

or a pharmaceutically acceptable salt thereof, wherein:

Y¹ is a bond, —CR₂—, —O—, —NR^{N}—, or —S—;

each instance of R is independently hydrogen, halogen, or optionally substituted alkyl; R^{N} is hydrogen, optionally substituted alkyl, optionally substituted acyl, or a nitrogen protecting group;

provided that when Y¹ is a bond or —CH₂—, R^{O} is not methyl.

In certain embodiments, when L¹ is —CR₂—, R^{O} is not optionally substituted alkyl. In certain embodiments, when L¹ is —CR₂—, R^{O} is hydrogen. In certain embodiments, when L¹ is —CH₂—, R^{O} is not optionally substituted alkyl. In certain embodiments, when L¹ is —CH₂—, R^{O} is hydrogen.

In certain embodiments, the PEG lipid of Formula (PVIII) is of one of the following formulae:

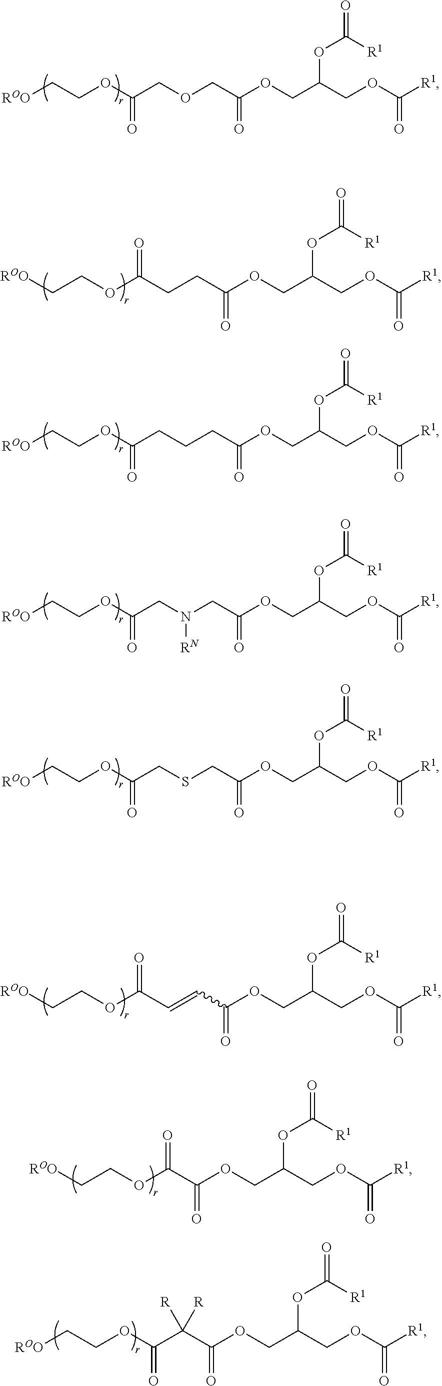

or a pharmaceutically acceptable salt thereof, wherein:

each instance of R is independently hydrogen, halogen, or optionally substituted alkyl.

In certain embodiments, the PEG lipid of Formula (PVIII) is of one of the following formulae:

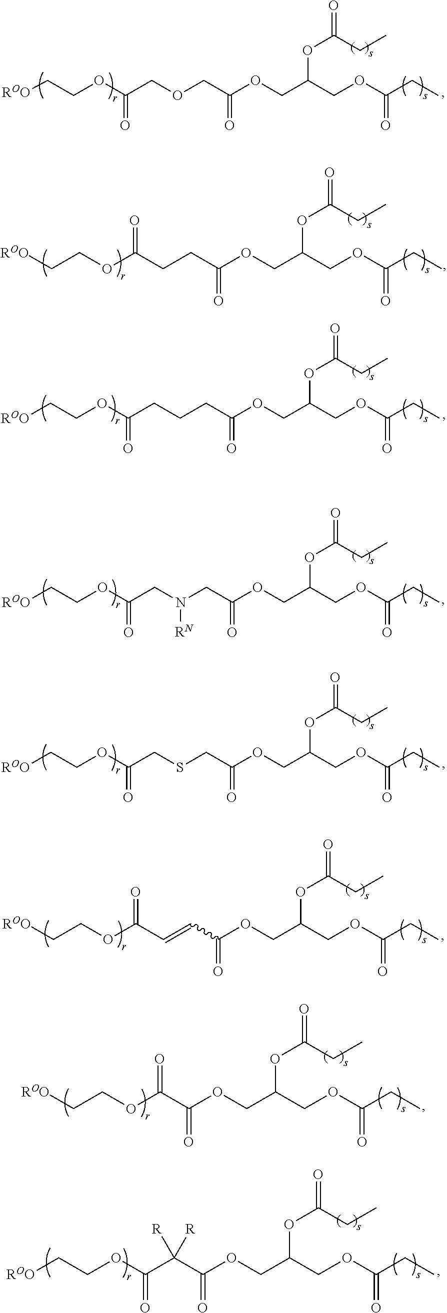

or a pharmaceutically acceptable salt thereof; wherein:

each instance of R is independently hydrogen, halogen, or optionally substituted alkyl; and

each s is independently an integer from 5-25, inclusive.

In certain embodiments, the PEG lipid of Formula (PVIII) is of one of the following formulae:

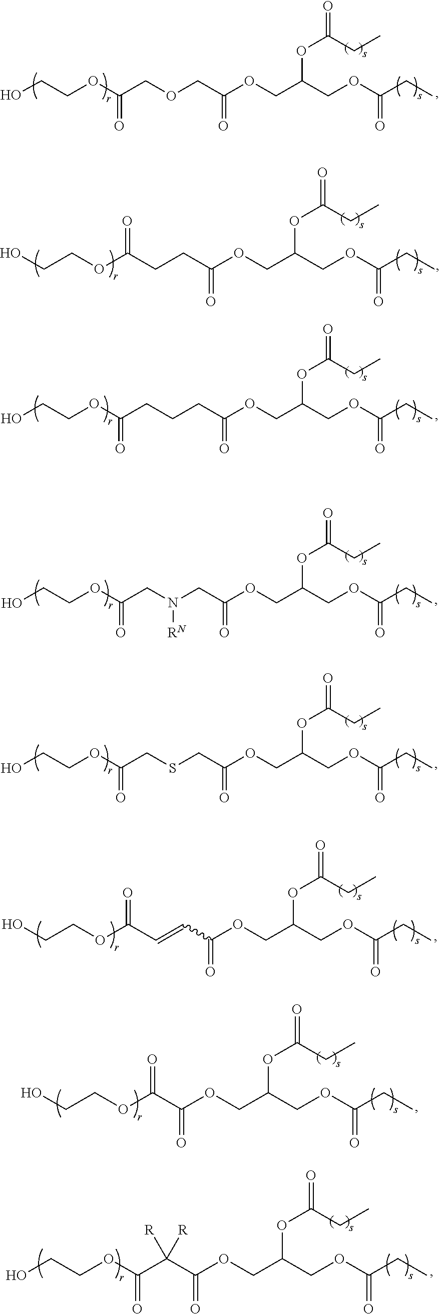

or a pharmaceutically acceptable salt thereof.

In certain embodiments, the PEG lipid of Formula (PVIII) is selected from the group consisting of:

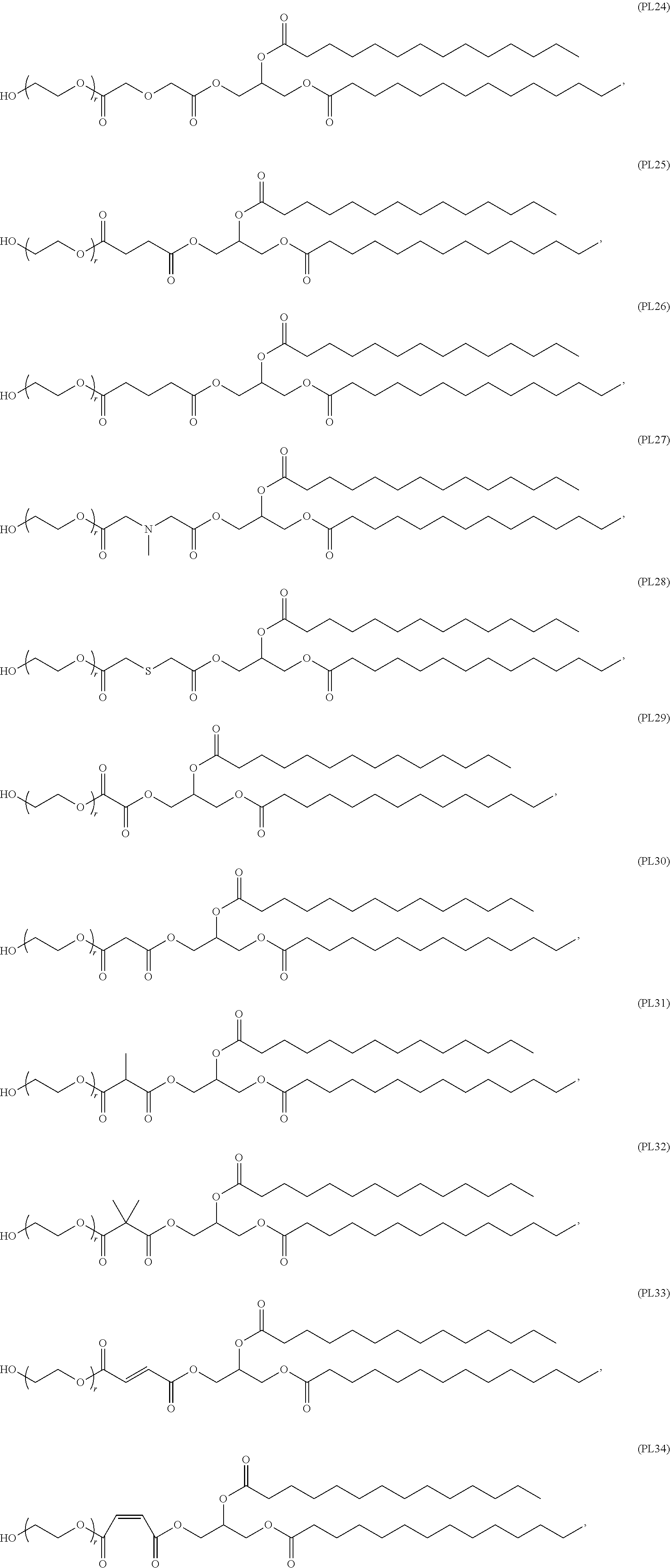

and pharmaceutically acceptable salts thereof.

In any of the foregoing or related aspects, a PEG lipid of the disclosure is featured wherein r is 40-50.

The LNPs provided herein, in certain embodiments, exhibit increased PEG shedding compared to existing LNP formulations comprising PEG lipids. “PEG shedding,” as used herein, refers to the cleavage of a PEG group from a PEG lipid. In many instances, cleavage of a PEG group from a PEG lipid occurs through serum-driven esterase-cleavage or hydrolysis. The PEG lipids provided herein, in certain embodiments, have been designed to control the rate of PEG shedding. In certain embodiments, an LNP provided herein exhibits greater than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits greater than 50% PEG shedding after about 6 hours in human serum. In certain embodiments, an LNP provided herein exhibits greater than 60% PEG shedding after about 6 hours in human serum. In certain embodiments, an LNP provided herein exhibits greater than 70% PEG shedding after about 6 hours in human serum. In certain embodiments, the LNP exhibits greater than 80% PEG shedding after about 6 hours in human serum. In certain embodiments, the LNP exhibits greater than 90% PEG shedding after about 6 hours in human serum. In certain embodiments, an LNP provided herein exhibits greater than 90% PEG shedding after about 6 hours in human serum.

In other embodiments, an LNP provided herein exhibits less than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits less than 60% PEG shedding after about 6 hours in human serum. In certain embodiments, an LNP provided herein exhibits less than 70% PEG shedding after about 6 hours in human serum. In certain embodiments, an LNP provided herein exhibits less than 80% PEG shedding after about 6 hours in human serum.

In addition to the PEG lipids provided herein, the LNP may comprise one or more additional lipid components. In certain embodiments, the PEG lipids are present in the LNP in a molar ratio of 0.15-15% with respect to other lipids. In certain embodiments, the PEG lipids are present in a molar ratio of 0.15-5% with respect to other lipids. In certain embodiments, the PEG lipids are present in a molar ratio of 1-5% with respect to other lipids. In certain embodiments, the PEG lipids are present in a molar ratio of 0.15-2% with respect to other lipids. In certain embodiments, the PEG lipids are present in a molar ratio of 1-2% with respect to other lipids. In certain embodiments, the PEG lipids are present in a molar ratio of approximately 1%, 1.1%, 1.2%, 1.3%, 1.4%, 1.5%, 1.6%, 1.7%, 1.8%, 1.9%, or 2% with respect to other lipids. In certain embodiments, the PEG lipids are present in a molar ratio of approximately 1.5% with respect to other lipids.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

Exemplary Synthesis:

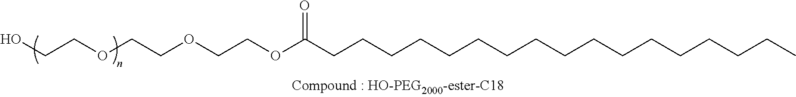

To a nitrogen filled flask containing palladium on carbon (10 wt. %, 74 mg, 0.070 mmol) was added Benzyl-PEG2000-ester-C₁₈ (822 mg, 0.35 mmol) and MeOH (20 mL). The flask was evacuated and backfilled with H₂ three times, and allowed to stir at RT and 1 atm H₂ for 12 hours. The mixture was filtered through celite, rinsing with DCM, and the filtrate was concentrated in vacuo to provide the desired product (692 mg, 88%). Using this methodology n=40-50. In one embodiment, n of the resulting polydispersed mixture is referred to by the average, 45.

For example, the value of r can be determined on the basis of a molecular weight of the PEG moiety within the PEG lipid. For example, a molecular weight of 2,000 (e.g., PEG2000) corresponds to a value of n of approximately 45. For a given composition, the value for n can connote a distribution of values within an art-accepted range, since polymers are often found as a distribution of different polymer chain lengths. For example, a skilled artisan understanding the polydispersity of such polymeric compositions would appreciate that an n value of 45 (e.g., in a structural formula) can represent a distribution of values between 40-50 in an actual PEG-containing composition, e.g., a DMG PEG200 peg lipid composition.

In some aspects, an immune cell delivery lipid of the pharmaceutical compositions disclosed herein does not comprise a PEG-lipid.

In one embodiment, an immune cell delivery LNP of the disclosure comprises a PEG-lipid. In one embodiment, the PEG lipid is not PEG DMG. In some aspects, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some aspects, the PEG lipid is selected from the group consisting of PEG-c-DOMG, PEG-DMG, PEG-DLPE, PEG-DMPE, PEG-DPPC and PEG-DSPE lipid. In other aspects, the PEG-lipid is PEG-DMG.

In one embodiment, an immune cell delivery LNP of the disclosure comprises a PEG-lipid which has a chain length longer than about 14 or than about 10, if branched.

In one embodiment, the PEG lipid is a compound selected from the group consisting of any of Compound Nos. P415, P416, P417, P 419, P 420, P 423, P 424, P 428, P L1, P L2, P L16, P L17, P L18, P L19, P L22 and P L23. In one embodiment, the PEG lipid is a compound selected from the group consisting of any of Compound Nos. P415, P417, P 420, P 423, P 424, P 428, P L1, P L2, P L16, P L17, P L18, P L19, P L22 and P L23.

In one embodiment, a PEG lipid is selected from the group consisting of: Cmpd 428, PL16, PL17, PL 18, PL19, PL 1, and PL 2.

Immune Cell Delivery Potentiating Lipids

An effective amount of the immune cell delivery potentiating lipid in an LNP enhances delivery of the agent to an immune cell (e.g., a human or primate immune cell) relative to an LNP lacking the immune cell delivery potentiating lipid, thereby creating an immune cell delivery LNP. Immune cell delivery potentiating lipids can be characterized in that, when present in an LNP, they promote delivery of the agent present in the LNP to immune cells as compared to a control LNP lacking the immune cell delivery potentiating lipid.

In one embodiment, the presence of at least one immune cell delivery potentiating lipid in an LNP results in an increase in the percentage of LNPs associated with immune cells as compared to a control LNP lacking at least one immune cell delivery potentiating lipid. In another embodiment, the presence of at least one immune cell delivery potentiating lipid in an LNP results in an increase in the delivery of a nucleic acid molecule agent to immune cells as compared to a control LNP lacking the immune cell delivery potentiating lipid. In one embodiment, the presence of at least one immune cell delivery potentiating lipid in an LNP results in an increase in the delivery of a nucleic acid molecule agent to B cells as compared to a control LNP lacking the immune cell delivery potentiating lipid. In particular, in one embodiment, the presence of at least one immune cell delivery potentiating lipid in an LNP results in an increase in the delivery of a nucleic acid molecule agent to myeloid cells as compared to a control LNP lacking the immune cell delivery potentiating lipid. In one embodiment, the presence of at least one immune cell delivery potentiating lipid in an LNP results in an increase in the delivery of a nucleic acid molecule agent to T cells as compared to a control LNP lacking the immune cell delivery potentiating lipid.

In one embodiment, the presence of at least one immune cell delivery potentiating lipid in an LNP results in an increase in the percentage of LNPs binding to C₁q as compared to a control LNP lacking at least one immune cell delivery potentiating lipid. In one embodiment, the presence of at least one immune cell delivery potentiating lipid in an LNP results in an increase in the percentage of C₁q-bound LNPs taken up by immune cells (e.g., opsonized by immune cells) as compared to a control LNP lacking at least one immune cell delivery potentiating lipid.

In one embodiment, when the nucleic acid molecule is an mRNA, the presence of at least one immune cell delivery potentiating lipid results in at least about 2-fold greater expression of a protein molecule encoded by the mRNA in immune cells (e.g., a T cells, B cells, monocytes) as compared to a control LNP lacking the immune cell delivery potentiating lipid.

In one embodiment, an immune cell delivery potentiating lipid is an ionizable lipid. In any of the foregoing or related aspects, the ionizable lipid (denoted by I) of the LNP of the disclosure comprises a compound included in any e.g. a compound having any of Formula (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8) and/or any of Compounds X, Y, I 48, I 50, I 109, I 111, I 113, I 181, I 182, I 244, I 292, I 301, I 321, I 322, I 326, I 328, I 330, I 331, I 332 or I M.

In one embodiment, an immune cell delivery potentiating lipid is an ionizable lipid. In any of the foregoing or related aspects, the ionizable lipid of the LNP of the disclosure comprises a compound described herein as Compound X, Compound Y, Compound I-321, Compound I-292, Compound I-326, Compound I-182, Compound I-301, Compound I-48, Compound I-50, Compound I-328, Compound I-330, Compound I-109, Compound I-111 or Compound I-181.

In any of the foregoing or related aspects, the ionizable lipid of the LNP of the disclosure comprises at least one compound selected from the group consisting of: Compound Nos. I 18 (also referred to as Compound X), I 25 (also referred to as Compound Y), I 48, I 50, I 109, I 111, I 113, I 181, I 182, I 244, I 292, I 301, I 309, I 317, I 321, I 322, I 326, I 328, I 330, I 331, I 332, I 347, I 348, I 349, I 350, I 351 and I 352. In another embodiment, the ionizable lipid of the LNP of the disclosure comprises a compound selected from the group consisting of: Compound Nos. I 18 (also referred to as Compound X), I 25 (also referred to as Compound Y), I 48, I 50, I 109, I 111, I 181, I 182, I 292, I 301, I 321, I 326, I 328, and I 330. In another embodiment, the ionizable lipid of the LNP of the disclosure comprises a compound selected from the group consisting of: Compound Nos. I 182, I 301, I 321, and I 326.

It will be understood that in embodiments where the immune cell delivery potentiating lipid comprises an ionizable lipid, it may be the only ionizable lipid present in the LNP or it may be present as a blend with at least one additional ionizable lipid. That is to say that a blend of ionizable lipids (e.g., more than one that have immune cell delivery potentiating effects or one that has an immune cell delivery potentiating effect and at least one that does not) may be employed.

In one embodiment, an immune cell delivery potentiating lipid comprises a sterol. In another embodiment, an immune cell delivery potentiating lipid comprises a naturally occurring sterol. In another embodiment, an immune cell delivery potentiating lipid comprises a modified sterol. In one embodiment, an immune cell delivery potentiating lipid comprises one or more phytosterols. In one embodiment, the immune cell delivery potentiating lipid comprises a phytosterol/cholesterol blend.

In one embodiment, the immune cell delivery potentiating lipid comprises an effective amount of a phytosterol.

The term “phytosterol” refers to the group of plant based sterols and stanols that are phytosteroids including salts or esters thereof.

The term “sterol” refers to the subgroup of steroids also known as steroid alcohols. Sterols are usually divided into two classes: (1) plant sterols also known as “phytosterols”, and (2) animal sterols also known as “zoosterols” such as cholesterol. The term “stanol” refers to the class of saturated sterols, having no double bonds in the sterol ring structure.

The term “effective amount of phytosterol” is intended to mean an amount of one or more phytosterols in a lipid-based composition, including an LNP, that will elicit a desired activity (e.g., enhanced delivery, enhanced immune cell uptake, enhanced nucleic acid activity). In some embodiments, an effective amount of phytosterol is all or substantially all (i.e., about 99-100%) of the sterol in a lipid nanoparticle. In some embodiments, an effective amount of phytosterol is less than all or substantially all of the sterol in a lipid nanoparticle (less than about 99-100%), but greater than the amount of non-phytosterol sterol in the lipid nanoparticle. In some embodiments, an effective amount of phytosterol is greater than 50%, greater than 60%, greater than 70%, greater than 75%, greater than 80%, greater than 85%, greater than 90% or greater than 95% the total amount of sterol in a lipid nanoparticle. In some embodiments, an effective amount of phytosterol is 95-100%, 75-100%, or 50-100% of the total amount of sterol in a lipid nanoparticle.

In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

In some embodiments, the sitosterol is a beta-sitosterol.

In some embodiments, the beta-sitosterol has the formula:

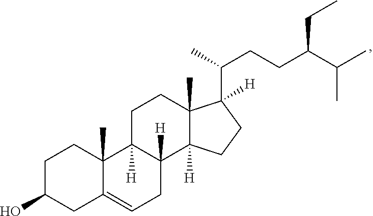

including analogs, salts or esters thereof.

In some embodiments, the sitosterol is a stigmasterol.

In some embodiments, the stigmasterol has the formula:

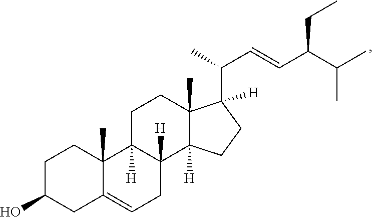

including analogs, salts or esters thereof.

In some embodiments, the sitosterol is a campesterol.

In some embodiments, the campesterol has the formula:

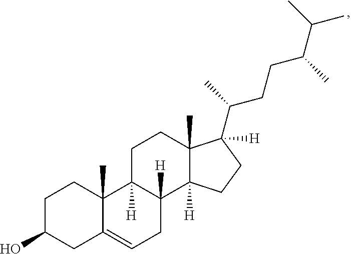

including analogs, salts or esters thereof.

In some embodiments, the sitosterol is a sitostanol.

In some embodiments, the sitostanol has the formula:

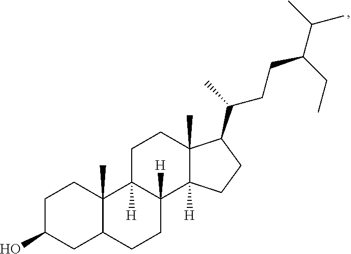

including analogs, salts or esters thereof.

In some embodiments, the sitosterol is a campestanol.

In some embodiments, the campestanol has the formula:

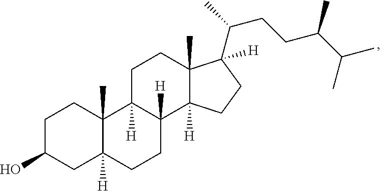

including analogs, salts or esters thereof.

In some embodiments, the sitosterol is a brassicasterol.

In some embodiments, the brassicasterol has the formula:

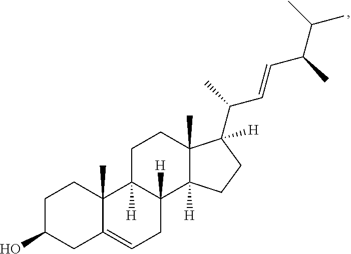

including analogs, salts or esters thereof.

In some embodiments, the sitosterol is a fucosterol.

In some embodiments, the fucosterol has the formula:

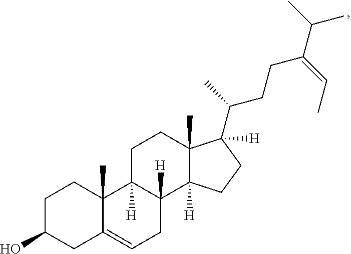

including analogs, salts or esters thereof.

In some embodiments, the phytosterol (e.g., beta-sitosterol) has a purity of greater than 70%. In some embodiments, the phytosterol (e.g., beta-sitosterol) has a purity of greater than 80%. In some embodiments, the phytosterol (e.g., beta-sitosterol) has a purity of greater than 90%. In some embodiments, the phytosterol (e.g., beta-sitosterol) has a purity of greater than 95%. In some embodiments, the phytosterol (e.g., beta-sitosterol) has a purity of greater than 97%, 98% or 99%.

In one embodiment, an immune cell delivery enhancing LNP comprises more than one type of structural lipid.

For example, in one embodiment, the immune cell delivery enhancing LNP comprises at least one immune cell delivery potentiating lipid which is a phytosterol. In one embodiment, the phytosterol is the only structural lipid present in the LNP. In another embodiment, the immune cell delivery LNP comprises a blend of structural lipids.

In one embodiment, the combined amount of the phytosterol and structural lipid (e.g., beta-sitosterol and cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %.

In one embodiment, the combined amount of the phytosterol and structural lipid (e.g., beta-sitosterol and cholesterol) in the lipid composition disclosed herein ranges from about 25 mol % to about 30 mol %, from about 30 mol % to about 35 mol %, or from about 35 mol % to about 40 mol %.

In one embodiment, the amount of the phytosterol and structural lipid (e.g., beta-sitosterol and cholesterol) in the lipid composition disclosed herein is about 24 mol %, about 29 mol %, about 34 mol %, or about 39 mol %.

In some embodiments, the combined amount of the phytosterol and structural lipid (e.g., beta-sitosterol and cholesterol) in the lipid composition disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

In some embodiments, the lipid nanoparticle comprises one or more phytosterols (e.g., beta-sitosterol) and one or more structural lipids (e.g. cholesterol). In some embodiments, the mol % of the structural lipid is between about 1% and 50% of the mol % of phytosterol present in the lipid nanoparticle. In some embodiments, the mol % of the structural lipid is between about 10% and 40% of the mol % of phytosterol present in the lipid-based composition (e.g., LNP). In some embodiments, the mol % of the structural lipid is between about 20% and 30% of the mol % of phytosterol present in the lipid-based composition (e.g., LNP). In some embodiments, the mol % of the structural lipid is about 30% of the mol % of phytosterol present in the lipid-based composition (e.g., lipid nanoparticle).

In some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17, 18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

In certain embodiments, the LNP comprises 50% ionizable lipid, 10% helper lipid (e.g, phospholipid), 38.5% structural lipid, and 1.5% PEG lipid. In certain embodiments, the LNP comprises 50% ionizable lipid, 10% helper lipid (e.g, phospholipid), 38% structural lipid, and 2% PEG lipid. In certain embodiments, the LNP comprises 50% ionizable lipid, 20% helper lipid (e.g, phospholipid), 28.5% structural lipid, and 1.5% PEG lipid. In certain embodiments, the LNP comprises 50% ionizable lipid, 20% helper lipid (e.g, phospholipid), 28% structural lipid, and 2% PEG lipid. In certain embodiments, the LNP comprises 40% ionizable lipid, 30% helper lipid (e.g, phospholipid), 28.5% structural lipid, and 1.5% PEG lipid. In certain embodiments, the LNP comprises 40% ionizable lipid, 30% helper lipid (e.g, phospholipid), 28% structural lipid, and 2% PEG lipid. In certain embodiments, the LNP comprises 45% ionizable lipid, 20% helper lipid (e.g, phospholipid), 33.5% structural lipid, and 1.5% PEG lipid. In certain embodiments, the LNP comprises 45% ionizable lipid, 20% helper lipid (e.g, phospholipid), 33% structural lipid, and 2% PEG lipid.

In one aspect, the immune cell delivery enhancing LNP comprises phytosterol and the LNP does not comprise an additional structural lipid. Accordingly, the structural lipid (sterol) component of the LNP consists of phytosterol. In another aspect, the immune cell delivery enhancing LNP comprises phytosterol and an additional structural lipid. Accordingly, the sterol component of the LNP comprise phytosterol and one or more additional sterols or structural lipids.

In any of the foregoing or related aspects, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), brassicasterol or stigmasterol, or combinations or blends thereof. In another embodiment, the structural lipid (e.g., sterol, such as a phytosterol or phytosterol/cholesterol blend) of the LNP of the disclosure comprises a compound selected from cholesterol, β-sitosterol, campesterol, β-sitostanol, brassicasterol, stigmasterol, β-sitosterol-d7, Compound S-30, Compound S-31, Compound S-32, or combinations or blends thereof. In another embodiment, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), Compound S-140, Compound S-144, brassicasterol (also referred to herein as Cmpd S 148) or Composition S-183 (˜40% Compound S-141, ˜25% Compound S-140, ˜25% Compound S-143 and ˜10% brassicasterol). In some embodiments, the structural lipid of the LNP of the disclosure comprises a compound described herein as Compound S-159, Compound S-160, Compound S-164, Compound S-165, Compound S-167, Compound S-170, Compound S-173 or Compound S-175.

In one embodiment, an immune cell delivery enhancing LNP comprises a non-cationic helper lipid, e.g., phospholipid. In any of the foregoing or related aspects, the non-cationic helper lipid (e.g, phospholipid) of the LNP of the disclosure comprises a compound described herein as DSPC, DMPE, DOPC or H-409. In one embodiment, the non-cationic helper lipid, e.g., phospholipid is DSPC. In other embodiments, the non-cationic helper lipid (e.g., phospholipid) of the LNP of the disclosure comprises a compound described herein as DSPC, DMPE, DOPC, DPPC, PMPC, H-409, H-418, H-420, H-421 or H-422.

In any of the foregoing or related aspects, the PEG lipid of the LNP of the disclosure comprises a compound described herein can be selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In another embodiment, the PEG lipid is selected from the group consisting of Compound Nos. P415, P416, P417, P 419, P 420, P 423, P 424, P 428, P L5, P L1, P L2, P L16, P L17, P L18, P L19, P L22, P L23, DMG, DPG and DSG. In another embodiment, the PEG lipid is selected from the group consisting of Cmpd 428, PL16, PL17, PL 18, PL19, P L5, PL 1, and PL 2.

In one embodiment, an immune cell delivery potentiating lipid comprises an effective amount of a combination of an ionizable lipid and a phytosterol.

In other embodiments, the disclosure provides lipid nanoparticles comprising one or more immune cell delivery potentiating lipids, wherein the LNP comprises Compound X as the ionizable lipid, DSPC as the phospholipid, cholesterol or a cholesterol/β-sitosterol blend as the structural lipid and Compound 428 as the PEG lipid. In various embodiments of these Compound X-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2; For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol. In another embodiment, the structural lipid is cholesterol/β-sitosterol at a total percentage of 38.5%, wherein the blend can comprise, for example: (i) 20% cholesterol and 18.5% β-sitosterol; or (ii) 10% cholesterol and 28.5% β-sitosterol.

In other embodiments, the disclosure provides lipid nanoparticles comprising one or more immune cell delivery potentiating lipids, wherein the LNP comprises Compound Y as the ionizable lipid, DSPC as the phospholipid, cholesterol or a cholesterol/β-sitosterol blend as the structural lipid and Compound 428 as the PEG lipid. In various embodiments of these Compound Y-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-182 as the ionizable lipid, DSPC as the phospholipid, cholesterol or a cholesterol/β-sitosterol blend as the structural lipid and Compound 428 as the PEG lipid. In various embodiments of these Compound I-182-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-321 as the ionizable lipid, DSPC as the phospholipid, cholesterol or a cholesterol/β-sitosterol blend as the structural lipid and Compound 428 as the PEG lipid. In various embodiments of these Compound I-321-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-292 as the ionizable lipid, DSPC as the phospholipid, cholesterol or a cholesterol/β-sitosterol blend as the structural lipid and Compound 428 as the PEG lipid. In various embodiments of these Compound I-292-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-326 as the ionizable lipid, DSPC as the phospholipid, cholesterol or a cholesterol/β-sitosterol blend as the structural lipid and Compound 428 as the PEG lipid. In various embodiments of these Compound I-326-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-301 as the ionizable lipid, DSPC as the phospholipid, cholesterol or a cholesterol/β-sitosterol blend as the structural lipid and Compound 428 as the PEG lipid. In various embodiments of these Compound I-301-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-48 as the ionizable lipid, DSPC as the phospholipid, cholesterol or a cholesterol/β-sitosterol blend as the structural lipid and Compound 428 as the PEG lipid. In various embodiments of these Compound I-48-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-50 as the ionizable lipid, DSPC as the phospholipid, cholesterol or a cholesterol/β-sitosterol blend as the structural lipid and Compound 428 as the PEG lipid. In various embodiments of these Compound I-50-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-328 as the ionizable lipid, DSPC as the phospholipid, cholesterol or a cholesterol/β-sitosterol blend as the structural lipid and Compound 428 as the PEG lipid. In various embodiments of these Compound I-328-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-330 as the ionizable lipid, DSPC as the phospholipid, cholesterol or a cholesterol/β-sitosterol blend as the structural lipid and Compound 428 as the PEG lipid. In various embodiments of these Compound I-330-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-109 as the ionizable lipid, DSPC as the phospholipid, cholesterol or a cholesterol/β-sitosterol blend as the structural lipid and Compound 428 as the PEG lipid. In various embodiments of these Compound I-109-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-111 as the ionizable lipid, DSPC as the phospholipid, cholesterol or a cholesterol/β-sitosterol blend as the structural lipid and Compound 428 as the PEG lipid. In various embodiments of these Compound I-111-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-181 as the ionizable lipid, DSPC as the phospholipid, cholesterol or a cholesterol/β-sitosterol blend as the structural lipid and Compound 428 as the PEG lipid. In various embodiments of these Compound I-181-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises any of Compounds X, Y, I-321, 1-292, 1-326, 1-182, 1-301, 1-48, 1-50, 1-328, 1-330, 1-109, I-111 or 1-181 as the ionizable lipid; DSPC as the phospholipid; cholesterol, a cholesterol/β-sitosterol blend, a β-sitosterol/β-sitostanol blend, a β-sitosterol/camposterol blend, a β-sitosterol/β-sitostanol/camposterol blend, a cholesterol/camposterol blend, a cholesterol/β-sitostanol blend, a cholesterol/β-sitostanol/camposterol blend or a cholesterol/β-sitosterol/β-sitostanol/camposterol blend as the structural lipid; and Compound 428 as the PEG lipid. In various embodiments of these compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2; (v) 40:18.5:40:1.5; or (vi) 45:20:33.5:1.5. In one embodiment, for the structural lipid component, the LNP can comprise, for example, 40% structural lipid composed of (i) 10% cholesterol and 30% β-sitosterol; (ii) 10% cholesterol and 30% campesterol; (iii) 10% cholesterol and 30% β-sitostanol; (iv) 10% cholesterol, 20% β-sitosterol and 10% campesterol; (v) 10% cholesterol, 20% β-sitosterol and 10% β-sitostanol; (vi) 10% cholesterol, 10% β-sitosterol and 20% campesterol; (vii) 10% cholesterol, 10% β-sitosterol and 20% campesterol; (viii) 10% cholesterol, 20% campesterol and 10% β-sitostanol; (ix) 10% cholesterol, 10% campesterol and 20% β-sitostanol; or (x) 10% cholesterol, 10% β-sitosterol, 10% campesterol and 10% β-sitostanol. In another embodiment, for the structural lipid component, the LNP can comprise, for example, 33.5% structural lipid composed of (i) 33.5% cholesterol; (ii) 18.5% cholesterol, 15% β-sitosterol; (iii) 18.5% cholesterol, 15% campesterol; or (iv) 18.5% cholesterol, 15% campesterol.

In other embodiments, the disclosure provides lipid nanoparticles comprising one or more immune cell delivery potentiating lipids, wherein the LNP comprises Compound I-301, Compound I-321 or Compound I-326 as the ionizable lipid; DSPC as the phospholipid; cholesterol or a cholesterol/β-sitosterol blend as the structural lipid; and Compound 428 as the PEG lipid. In one embodiment, the LNP enhances delivery to T cells (e.g., CD3+ T cells).

In other embodiment, the disclosure provides lipid nanoparticles comprising one or more immune cell delivery potentiating lipids, wherein the LNP comprises Compound X, Compound I-109, Compound I-111, Compound I-181, Compound I-182 or Compound I-244, wherein the LNP enhances delivery to monocytes. The other components of the LNP can be selected from those disclosed herein, for example DSPC as the phospholipid; cholesterol or a cholesterol/β-sitosterol blend as the structural lipid; and Compound 428 as the PEG lipid.

In other embodiment, the disclosure provides lipid nanoparticles comprising one or more immune cell delivery potentiating lipids, wherein the LNP comprises camposterol, β-sitostanol or stigmasterol as the structural lipid, wherein the LNP enhances delivery to monocytes. The other components of the LNP can be selected from those disclosed herein, for example Compound X, Compound I-109, Compound I-111, Compound I-181, Compound I-182 or Compound I-244 as the ionizable lipid; DSPC as the phospholipid; and Compound 428 as the PEG lipid.

In other embodiment, the disclosure provides lipid nanoparticles comprising one or more immune cell delivery potentiating lipids, wherein the LNP comprises DOPC, DMPE or H-409 as the helper lipid (e.g., phospholipid), wherein the LNP enhances delivery to monocytes. The other components of the LNP can be selected from those disclosed herein, for example Compound X, Compound I-109, Compound I-111, Compound I-181, Compound I-182 or Compound I-244 as the ionizable lipid; cholesterol, a cholesterol/β-sitosterol blend, camposterol, β-sitostanol or stigmasterol as the structural lipid; and Compound 428 as the PEG lipid.

### Exemplary Additional LNP Components

Surfactants

In certain embodiments, the lipid nanoparticles of the disclosure optionally includes one or more surfactants.

In certain embodiments, the surfactant is an amphiphilic polymer. As used herein, an amphiphilic “polymer” is an amphiphilic compound that comprises an oligomer or a polymer. For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10⁻⁴ M in water at about 30° C. and atmospheric pressure.

For example, amphiphilic polymer has a critical micelle concentration (CMC) ranging between about 0.1×10⁻⁴ M and about 1.3×10⁻⁴ M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

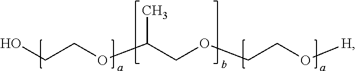

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof.

For example, the polyethylene glycol ether is a compound of Formula (VIII):

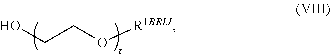

or a salt or isomer thereof, wherein:

t is an integer between 1 and 100;

R^{1BRIJ} independently is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NRNC(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group

In some embodiment, R^{1BRIJ} is C₁₈ alkyl. For example, the polyethylene glycol ether is a compound of Formula (VIII-a):

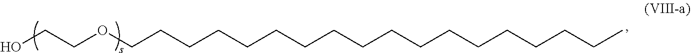

or a salt or isomer thereof.

In some embodiments, R^{1BRIJ} is C₁₈ alkenyl. For example, the polyethylene glycol ether is a compound of Formula (VIII-b):

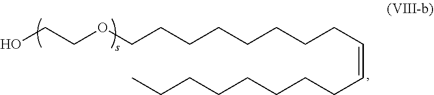

or a salt or isomer thereof

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

Adjuvants

In some embodiments, an LNP of the disclosure optionally includes one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

Other Components

An LNP of the disclosure may optionally include one or more components in addition to those described in the preceding sections. For example, a lipid nanoparticle may include one or more small hydrophobic molecules such as a vitamin (e.g., vitamin A or vitamin E) or a sterol.

Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(-methyl-2-oxazoline) (PMOX), poly(-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin (34, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### LNP Compositions

A lipid nanoparticle described herein may be designed for one or more specific applications or targets. The elements of a lipid nanoparticle and their relative amounts may be selected based on a particular application or target, and/or based on the efficacy, toxicity, expense, ease of use, availability, or other feature of one or more elements. Similarly, the particular formulation of a lipid nanoparticle may be selected for a particular application or target according to, for example, the efficacy and toxicity of particular combinations of elements. The efficacy and tolerability of a lipid nanoparticle formulation may be affected by the stability of the formulation.

The LNPs of the disclosure comprise at least one immune cell delivery potentiating lipid. The subject LNPs comprise: an effective amount of an immune cell delivery potentiating lipid as a component of an LNP, wherein the LNP comprises an (i) ionizable lipid; (ii) cholesterol or other structural lipid; (iii) a non-cationic helper lipid or phospholipid; a (iv) PEG lipid and (v) an agent (e.g, an nucleic acid molecule) encapsulated in and/or associated with the LNP, wherein the effective amount of the immune cell delivery potentiating lipid enhances delivery of the agent to an immune cell (e.g., a human or primate immune cell) relative to an LNP lacking the immune cell delivery potentiating lipid.

The elements of the various components may be provided in specific fractions, e.g., mole percent fractions.

For example, in any of the foregoing or related aspects, the LNP of the disclosure comprises a structural lipid or a salt thereof. In some aspects, the structural lipid is cholesterol or a salt thereof. In further aspects, the mol % cholesterol is between about 1% and 50% of the mol % of phytosterol present in the LNP. In other aspects, the mol % cholesterol is between about 10% and 40% of the mol % of phytosterol present in the LNP. In some aspects, the mol % cholesterol is between about 20% and 30% of the mol % of phytosterol present in the LNP. In further aspects, the mol % cholesterol is about 30% of the mol % of phytosterol present in the LNP.

In any of the foregoing or related aspects, the LNP of the disclosure comprises about 30 mol % to about 60 mol % ionizable lipid, about 0 mol % to about 30 mol % phospholipid, about 18.5 mol % to about 48.5 mol % sterol, and about 0 mol % to about 10 m of % PEG lipid.

In any of the foregoing or related aspects, the LNP of the disclosure comprises about 35 mol % to about 55 mol % ionizable lipid, about 5 mol % to about 25 mol % phospholipid, about 30 mol % to about 40 mol % sterol, and about 0 mol % to about 10 mol % PEG lipid.

In any of the foregoing or related aspects, the LNP of the disclosure comprises about 50 mol % ionizable lipid, about 10 mol % phospholipid, about 38.5 mol % sterol, and about 1.5 mol % PEG lipid.

In certain embodiments, the ionizable lipid component of the lipid nanoparticle includes about 30 mol % to about 60 mol % ionizable lipid, about 0 mol % to about 30 mol % non-cationic helper lipid, about 18.5 mol % to about 48.5 mol % phytosterol optionally including one or more structural lipids, and about 0 mol % to about 10 mol % of PEG lipid, provided that the total mol % does not exceed 100%. In some embodiments, the ionizable lipid component of the lipid nanoparticle includes about 35 mol % to about 55 mol % ionizable lipid, about 5 mol % to about 25 mol % non-cationic helper lipid, about 30 mol % to about 40 mol % phytosterol optionally including one or more structural lipids, and about 0 mol % to about 10 mol % of PEG lipid. In a particular embodiment, the lipid component includes about 50 mol % ionizable lipid, about 10 mol % non-cationic helper lipid, about 38.5 mol % phytosterol optionally including one or more structural lipids, and about 1.5 mol % of PEG lipid. In another particular embodiment, the lipid component includes about 40 mol % ionizable lipid, about 20 mol % non-cationic helper lipid, about 38.5 mol % phytosterol optionally including one or more structural lipids, and about 1.5 mol % of PEG lipid. In some embodiments, the phytosterol may be beta-sitosterol, the non-cationic helper lipid may be a phospholipid such as DOPE, DSPC or a phospholipid substitute such as oleic acid. In other embodiments, the PEG lipid may be PEG-DMG and/or the structural lipid may be cholesterol.

In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and a structural lipid, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and cholesterol, and about 0 mol % to about 10 mol % PEG lipid.

In some aspects, the LNP of the disclosure comprises about 35 mol % to about 55 mol % ionizable lipid, about 5 mol % to about 25 mol % non-cationic helper lipid, about 30 mol % to about 40 mol % phytosterol, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and a structural lipid, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and cholesterol, and about 0 mol % to about 10 mol % PEG lipid.

In some aspects, the LNP of the disclosure comprises about 50 mol % ionizable lipid, about 10 mol % non-cationic helper lipid, about 38.5 mol % phytosterol, and about 1.5 mol % PEG lipid. In some aspects, the LNP of the disclosure comprises about 50 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 50 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 38.5 mol % phytosterol, and about 1.5 mol % PEG lipid. In some aspects, the LNP of the disclosure comprises about 40 mol % ionizable lipid, about 20 mol % non-cationic helper lipid, about 38.5 mol % phytosterol and a structural lipid, and about 1.5 mol % PEG lipid. In some aspects, the LNP of the disclosure comprises about 40 mol % ionizable lipid, about 20 mol % non-cationic helper lipid, about 38.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 38.5 mol % phytosterol, and about 1.5 mol % PEG lipid. In some aspects, the LNP of the disclosure comprises about 45 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 55 mol % ionizable lipid, about 5 mol % non-cationic helper lipid, about 38.5 mol % phytosterol, and about 1.5 mol % PEG lipid. In some aspects, the LNP of the disclosure comprises about 55 mol % ionizable lipid, about 5 mol % non-cationic helper lipid, about 38.5 mol % phytosterol and a structural lipid, and about 1.5 mol % PEG lipid. In some aspects, the LNP of the disclosure comprises about 55 mol % ionizable lipid, about 5 mol % non-cationic helper lipid, about 38.5 mol % phytosterol and cholesterol, and about 1.5 mol % PEG lipid.

In some aspects, the LNP of the disclosure comprises about 60 mol % ionizable lipid, about 5 mol % non-cationic helper lipid, about 33.5 mol % phytosterol, and about 1.5 mol % PEG lipid. In some aspects, the LNP of the disclosure comprises about 60 mol % ionizable lipid, about 5 mol % non-cationic helper lipid, about 33.5 mol % phytosterol and a structural lipid, and about 1.5 mol % PEG lipid. In some aspects, the LNP of the disclosure comprises about 60 mol % ionizable lipid, about 5 mol % non-cationic helper lipid, about 33.5 mol % phytosterol and cholesterol, and about 1.5 mol % PEG lipid.

In some aspects, the LNP of the disclosure comprises about 45 mol % ionizable lipid, about 20 mol % non-cationic helper lipid, about 33.5 mol % phytosterol, and about 1.5 mol % PEG lipid. In some aspects, the LNP of the disclosure comprises about 45 mol % ionizable lipid, about 20 mol % non-cationic helper lipid, about 33.5 mol % phytosterol and a structural lipid, and about 1.5 mol % PEG lipid. In some aspects, the LNP of the disclosure comprises about 45 mol % ionizable lipid, about 20 mol % non-cationic helper lipid, about 33.5 mol % phytosterol and cholesterol, and about 1.5 mol % PEG lipid.

In some aspects, the LNP of the disclosure comprises about 50 mol % ionizable lipid, about 20 mol % non-cationic helper lipid, about 28.5 mol % phytosterol, and about 1.5 mol % PEG lipid. In some aspects, the LNP of the disclosure comprises about 50 mol % ionizable lipid, about 20 mol % non-cationic helper lipid, about 28.5 mol % phytosterol and a structural lipid, and about 1.5 mol % PEG lipid. In some aspects, the LNP of the disclosure comprises about 50 mol % ionizable lipid, about 20 mol % non-cationic helper lipid, about 28.5 mol % phytosterol and cholesterol, and about 1.5 mol % PEG lipid.

In some aspects, the LNP of the disclosure comprises about 55 mol % ionizable lipid, about 20 mol % non-cationic helper lipid, about 23.5 mol % phytosterol, and about 1.5 mol % PEG lipid. In some aspects, the LNP of the disclosure comprises about 55 mol % ionizable lipid, about 20 mol % non-cationic helper lipid, about 23.5 mol % phytosterol and a structural lipid, and about 1.5 mol % PEG lipid. In some aspects, the LNP of the disclosure comprises about 55 mol % ionizable lipid, about 20 mol % non-cationic helper lipid, about 23.5 mol % phytosterol and cholesterol, and about 1.5 mol % PEG lipid.

In some aspects, the LNP of the disclosure comprises about 60 mol % ionizable lipid, about 20 mol % non-cationic helper lipid, about 18.5 mol % phytosterol, and about 1.5 mol % PEG lipid. In some aspects, the LNP of the disclosure comprises about 60 mol % ionizable lipid, about 20 mol % non-cationic helper lipid, about 18.5 mol % phytosterol and a structural lipid, and about 1.5 mol % PEG lipid. In some aspects, the LNP of the disclosure comprises about 60 mol % ionizable lipid, about 20 mol % non-cationic helper lipid, about 18.5 mol % phytosterol and cholesterol, and about 1.5 mol % PEG lipid.

In some aspects, the LNP of the disclosure comprises about 40 mol % ionizable lipid, about 15 mol % non-cationic helper lipid, about 43.5 mol % phytosterol, and about 1.5 mol % PEG lipid. In some aspects, the LNP of the disclosure comprises about 40 mol % ionizable lipid, about 15 mol % non-cationic helper lipid, about 43.5 mol % phytosterol and a structural lipid, and about 1.5 mol % PEG lipid. In some aspects, the LNP of the disclosure comprises about 40 mol % ionizable lipid, about 15 mol % non-cationic helper lipid, about 43.5 mol % phytosterol and cholesterol, and about 1.5 mol % PEG lipid.

In some aspects, the LNP of the disclosure comprises about 50 mol % ionizable lipid, about 15 mol % non-cationic helper lipid, about 33.5 mol % phytosterol, and about 1.5 mol % PEG lipid. In some aspects, the LNP of the disclosure comprises about 50 mol % ionizable lipid, about 15 mol % non-cationic helper lipid, about 33.5 mol % phytosterol and a structural lipid, and about 1.5 mol % PEG lipid. In some aspects, the LNP of the disclosure comprises about 50 mol % ionizable lipid, about 15 mol % non-cationic helper lipid, about 33.5 mol % phytosterol and cholesterol, and about 1.5 mol % PEG lipid.

In some aspects, the LNP of the disclosure comprises about 55 mol % ionizable lipid, about 15 mol % non-cationic helper lipid, about 28.5 mol % phytosterol, and about 1.5 mol % PEG lipid. In some aspects, the LNP of the disclosure comprises about 55 mol % ionizable lipid, about 15 mol % non-cationic helper lipid, about 28.5 mol % phytosterol and a structural lipid, and about 1.5 mol % PEG lipid. In some aspects, the LNP of the disclosure comprises about 55 mol % ionizable lipid, about 15 mol % non-cationic helper lipid, about 28.5 mol % phytosterol and cholesterol, and about 1.5 mol % PEG lipid.

In some aspects, the LNP of the disclosure comprises about 60 mol % ionizable lipid, about 15 mol % non-cationic helper lipid, about 23.5 mol % phytosterol, and about 1.5 mol % PEG lipid. In some aspects, the LNP of the disclosure comprises about 60 mol % ionizable lipid, about 15 mol % non-cationic helper lipid, about 23.5 mol % phytosterol and a structural lipid, and about 1.5 mol % PEG lipid. In some aspects, the LNP of the disclosure comprises about 60 mol % ionizable lipid, about 15 mol % non-cationic helper lipid, about 23.5 mol % phytosterol and cholesterol, and about 1.5 mol % PEG lipid.

In some aspects, the LNP of the disclosure comprises about 40 mol % ionizable lipid, about 10 mol % non-cationic helper lipid, about 48.5 mol % phytosterol, and about 1.5 mol % PEG lipid. In some aspects, the LNP of the disclosure comprises about 40 mol % ionizable lipid, about 10 mol % non-cationic helper lipid, about 48.5 mol % phytosterol and a structural lipid, and about 1.5 mol % PEG lipid. In some aspects, the LNP of the disclosure comprises about 40 mol % ionizable lipid, about 10 mol % non-cationic helper lipid, about 48.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 43.5 mol % phytosterol, and about 1.5 mol % PEG lipid. In some aspects, the LNP of the disclosure comprises about 45 mol % ionizable lipid, about 10 mol % non-cationic helper lipid, about 43.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 43.5 mol % phytosterol and cholesterol, and about 1.5 mol % PEG lipid.

In some aspects, the LNP of the disclosure comprises about 55 mol % ionizable lipid, about 10 mol % non-cationic helper lipid, about 33.5 mol % phytosterol, and about 1.5 mol % PEG lipid. In some aspects, the LNP of the disclosure comprises about 55 mol % ionizable lipid, about 10 mol % non-cationic helper lipid, about 33.5 mol % phytosterol and a structural lipid, and about 1.5 mol % PEG lipid. In some aspects, the LNP of the disclosure comprises about 55 mol % ionizable lipid, about 10 mol % non-cationic helper lipid, about 33.5 mol % phytosterol and cholesterol, and about 1.5 mol % PEG lipid.

In some aspects, the LNP of the disclosure comprises about 60 mol % ionizable lipid, about 10 mol % non-cationic helper lipid, about 28.5 mol % phytosterol, and about 1.5 mol % PEG lipid. In some aspects, the LNP of the disclosure comprises about 60 mol % ionizable lipid, about 10 mol % non-cationic helper lipid, about 28.5 mol % phytosterol and a structural lipid, and about 1.5 mol % PEG lipid. In some aspects, the LNP of the disclosure comprises about 60 mol % ionizable lipid, about 10 mol % non-cationic helper lipid, about 28.5 mol % phytosterol and cholesterol, and about 1.5 mol % PEG lipid.

In some aspects, the LNP of the disclosure comprises about 40 mol % ionizable lipid, about 5 mol % non-cationic helper lipid, about 53.5 mol % phytosterol, and about 1.5 mol % PEG lipid. In some aspects, the LNP of the disclosure comprises about 40 mol % ionizable lipid, about 5 mol % non-cationic helper lipid, about 53.5 mol % phytosterol and a structural lipid, and about 1.5 mol % PEG lipid. In some aspects, the LNP of the disclosure comprises about 40 mol % ionizable lipid, about 5 mol % non-cationic helper lipid, about 53.5 mol % phytosterol and cholesterol, and about 1.5 mol % PEG lipid.

In some aspects, the LNP of the disclosure comprises about 45 mol % ionizable lipid, about 5 mol % non-cationic helper lipid, about 48.5 mol % phytosterol, and about 1.5 mol % PEG lipid. In some aspects, the LNP of the disclosure comprises about 45 mol % ionizable lipid, about 5 mol % non-cationic helper lipid, about 48.5 mol % phytosterol and a structural lipid, and about 1.5 mol % PEG lipid. In some aspects, the LNP of the disclosure comprises about 45 mol % ionizable lipid, about 5 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 5 mol % non-cationic helper lipid, about 43.5 mol % phytosterol, and about 1.5 mol % PEG lipid. In some aspects, the LNP of the disclosure comprises about 50 mol % ionizable lipid, about 5 mol % non-cationic helper lipid, about 43.5 mol % phytosterol and a structural lipid, and about 1.5 mol % PEG lipid. In some aspects, the LNP of the disclosure comprises about 50 mol ionizable lipid, about 5 mol % non-cationic helper lipid, about 43.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 40 mol % phytosterol, and about 0 mol % PEG lipid. In some aspects, the LNP of the disclosure comprises about 40 mol % ionizable lipid, about 20 mol % non-cationic helper lipid, about 40 mol % phytosterol and a structural lipid, and about 0 mol % PEG lipid. In some aspects, the LNP of the disclosure comprises about 40 mol % ionizable lipid, about 20 mol % non-cationic helper lipid, about 40 mol % phytosterol and cholesterol, and about 0 mol % PEG lipid.

In some aspects, the LNP of the disclosure comprises about 45 mol % ionizable lipid, about 20 mol % non-cationic helper lipid, about 35 mol % phytosterol, and about 0 mol % PEG lipid. In some aspects, the LNP of the disclosure comprises about 45 mol % ionizable lipid, about 20 mol % non-cationic helper lipid, about 35 mol % phytosterol and a structural lipid, and about 0 mol % PEG lipid. In some aspects, the LNP of the disclosure comprises about 45 mol % ionizable lipid, about 20 mol % non-cationic helper lipid, about 35 mol % phytosterol and cholesterol, and about 0 mol % PEG lipid.

In some aspects, the LNP of the disclosure comprises about 50 mol % ionizable lipid, about 20 mol % non-cationic helper lipid, about 30 mol % phytosterol, and about 0 mol % PEG lipid. In some aspects, the LNP of the disclosure comprises about 50 mol % ionizable lipid, about 20 mol % non-cationic helper lipid, about 30 mol % phytosterol and a structural lipid, and about 0 mol % PEG lipid. In some aspects, the LNP of the disclosure comprises about 50 mol % ionizable lipid, about 20 mol % non-cationic helper lipid, about 30 mol % phytosterol and cholesterol, and about 0 mol % PEG lipid.

In some aspects, the LNP of the disclosure comprises about 55 mol % ionizable lipid, about 20 mol % non-cationic helper lipid, about 25 mol % phytosterol, and about 0 mol % PEG lipid. In some aspects, the LNP of the disclosure comprises about 55 mol % ionizable lipid, about 20 mol % non-cationic helper lipid, about 25 mol % phytosterol and a structural lipid, and about 0 mol % PEG lipid. In some aspects, the LNP of the disclosure comprises about 55 mol % ionizable lipid, about 20 mol % non-cationic helper lipid, about 25 mol % phytosterol and cholesterol, and about 0 mol % PEG lipid.

In some aspects, the LNP of the disclosure comprises about 60 mol % ionizable lipid, about 20 mol % non-cationic helper lipid, about 20 mol % phytosterol, and about 0 mol % PEG lipid. In some aspects, the LNP of the disclosure comprises about 60 mol % ionizable lipid, about 20 mol % non-cationic helper lipid, about 20 mol % phytosterol and a structural lipid, and about 0 mol % PEG lipid. In some aspects, the LNP of the disclosure comprises about 60 mol % ionizable lipid, about 20 mol % non-cationic helper lipid, about 20 mol % phytosterol and cholesterol, and about 0 mol % PEG lipid.

In some aspects, the LNP of the disclosure comprises about 40 mol % ionizable lipid, about 15 mol % non-cationic helper lipid, about 45 mol % phytosterol, and about 0 mol % PEG lipid. In some aspects, the LNP of the disclosure comprises about 40 mol % ionizable lipid, about 15 mol % non-cationic helper lipid, about 45 mol % phytosterol and a structural lipid, and about 0 mol % PEG lipid. In some aspects, the LNP of the disclosure comprises about 40 mol % ionizable lipid, about 15 mol % non-cationic helper lipid, about 45 mol % phytosterol and cholesterol, and about 0 mol % PEG lipid.

In some aspects, the LNP of the disclosure comprises about 45 mol % ionizable lipid, about 15 mol % non-cationic helper lipid, about 40 mol % phytosterol, and about 0 mol % PEG lipid. In some aspects, the LNP of the disclosure comprises about 45 mol % ionizable lipid, about 15 mol % non-cationic helper lipid, about 40 mol % phytosterol and a structural lipid, and about 0 mol % PEG lipid. In some aspects, the LNP of the disclosure comprises about 45 mol % ionizable lipid, about 15 mol % non-cationic helper lipid, about 40 mol % phytosterol and cholesterol, and about 0 mol % PEG lipid.

In some aspects, the LNP of the disclosure comprises about 50 mol % ionizable lipid, about 15 mol % non-cationic helper lipid, about 35 mol % phytosterol, and about 0 mol % PEG lipid. In some aspects, the LNP of the disclosure comprises about 50 mol % ionizable lipid, about 15 mol % non-cationic helper lipid, about 35 mol % phytosterol and a structural lipid, and about 0 mol % PEG lipid. In some aspects, the LNP of the disclosure comprises about 50 mol % ionizable lipid, about 15 mol % non-cationic helper lipid, about 35 mol % phytosterol and cholesterol, and about 0 mol % PEG lipid.

In some aspects, the LNP of the disclosure comprises about 55 mol % ionizable lipid, about 15 mol % non-cationic helper lipid, about 30 mol % phytosterol, and about 0 mol % PEG lipid. In some aspects, the LNP of the disclosure comprises about 55 mol % ionizable lipid, about 15 mol % non-cationic helper lipid, about 30 mol % phytosterol and a structural lipid, and about 0 mol % PEG lipid. In some aspects, the LNP of the disclosure comprises about 55 mol % ionizable lipid, about 15 mol % non-cationic helper lipid, about 30 mol % phytosterol and cholesterol, and about 0 mol % PEG lipid.

In some aspects, the LNP of the disclosure comprises about 60 mol % ionizable lipid, about 15 mol % non-cationic helper lipid, about 25 mol % phytosterol, and about 0 mol % PEG lipid. In some aspects, the LNP of the disclosure comprises about 60 mol % ionizable lipid, about 15 mol % non-cationic helper lipid, about 25 mol % phytosterol and a structural lipid, and about 0 mol % PEG lipid. In some aspects, the LNP of the disclosure comprises about 60 mol % ionizable lipid, about 15 mol % non-cationic helper lipid, about 25 mol % phytosterol and cholesterol, and about 0 mol % PEG lipid.

In some aspects, the LNP of the disclosure comprises about 40 mol % ionizable lipid, about 10 mol % non-cationic helper lipid, about 50 mol % phytosterol, and about 0 mol % PEG lipid. In some aspects, the LNP of the disclosure comprises about 40 mol % ionizable lipid, about 10 mol % non-cationic helper lipid, about 50 mol % phytosterol and a structural lipid, and about 0 mol % PEG lipid. In some aspects, the LNP of the disclosure comprises about 40 mol % ionizable lipid, about 10 mol % non-cationic helper lipid, about 50 mol % phytosterol and cholesterol, and about 0 mol % PEG lipid.

In some aspects, the LNP of the disclosure comprises about 45 mol % ionizable lipid, about 10 mol % non-cationic helper lipid, about 45 mol % phytosterol, and about 0 mol % PEG lipid. In some aspects, the LNP of the disclosure comprises about 45 mol % ionizable lipid, about 10 mol % non-cationic helper lipid, about 45 mol % phytosterol and a structural lipid, and about 0 mol % PEG lipid. In some aspects, the LNP of the disclosure comprises about 45 mol % ionizable lipid, about 10 mol % non-cationic helper lipid, about 45 mol % phytosterol and cholesterol, and about 0 mol % PEG lipid.

In some aspects, the LNP of the disclosure comprises about 50 mol % ionizable lipid, about 0 mol % non-cationic helper lipid, about 48.5 mol % phytosterol, and about 1.5 mol % PEG lipid. In some aspects, the LNP of the disclosure comprises about 50 mol % ionizable lipid, about 0 mol % non-cationic helper lipid, about 48.5 mol % phytosterol and a structural lipid, and about 1.5 mol % PEG lipid. In some aspects, the LNP of the disclosure comprises about 50 mol ionizable lipid, about 0 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 10 mol % non-cationic helper lipid, about 40 mol % phytosterol, and about 0 mol % PEG lipid. In some aspects, the LNP of the disclosure comprises about 50 mol % ionizable lipid, about 10 mol % non-cationic helper lipid, about 40 mol % phytosterol and a structural lipid, and about 0 mol % PEG lipid. In some aspects, the LNP of the disclosure comprises about 50 mol % ionizable lipid, about 10 mol % non-cationic helper lipid, about 40 mol % phytosterol and cholesterol, and about 0 mol % PEG lipid.

In some aspects, the LNP of the disclosure comprises about 55 mol % ionizable lipid, about 10 mol % non-cationic helper lipid, about 35 mol % phytosterol, and about 0 mol % PEG lipid. In some aspects, the LNP of the disclosure comprises about 55 mol % ionizable lipid, about 10 mol % non-cationic helper lipid, about 35 mol % phytosterol and a structural lipid, and about 0 mol % PEG lipid. In some aspects, the LNP of the disclosure comprises about 55 mol % ionizable lipid, about 10 mol % non-cationic helper lipid, about 35 mol % phytosterol and cholesterol, and about 0 mol % PEG lipid.

In some aspects, the LNP of the disclosure comprises about 60 mol % ionizable lipid, about 10 mol % non-cationic helper lipid, about 30 mol % phytosterol, and about 0 mol % PEG lipid. In some aspects, the LNP of the disclosure comprises about 60 mol % ionizable lipid, about 10 mol % non-cationic helper lipid, about 30 mol % phytosterol and a structural lipid, and about 0 mol % PEG lipid. In some aspects, the LNP of the disclosure comprises about 60 mol % ionizable lipid, about 10 mol % non-cationic helper lipid, about 30 mol % phytosterol and cholesterol, and about 0 mol % PEG lipid.

In some aspects with respect to the embodiments herein, the phytosterol and a structural lipid components of a LNP of the disclosure comprises between about 10:1 and 1:10 phytosterol to structural lipid, such as about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9 and 1:10 phytosterol to structural lipid (e.g. beta-sitosterol to cholesterol).

In some embodiments, the phytosterol component of the LNP is a blend of the phytosterol and a structural lipid, such as cholesterol, wherein the phytosterol (e.g., beta-sitosterol) and the structural lipid (e.g., cholesterol) are each present at a particular mol %. For example, in some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17, 18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

Lipid nanoparticles of the disclosure may be designed for one or more specific applications or targets. For example, the subject lipid nanoparticles may optionally be designed to further enhance delivery of a nucleic acid molecule, such as an RNA, to a particular immune cell (e.g., lymphoid cell or myeloid cell), tissue, organ, or system or group thereof in a mammal's, e.g., a human's body. Physiochemical properties of lipid nanoparticles may be altered in order to increase selectivity for particular bodily targets. For instance, particle sizes may be adjusted to promote immune cell uptake. As set forth above, the nucleic acid molecule included in a lipid nanoparticle may also be selected based on the desired delivery to immune cells. For example, a nucleic acid molecule may be selected for a particular indication, condition, disease, or disorder and/or for delivery to a particular cell, tissue, organ, or system or group thereof (e.g., localized or specific delivery).

In certain embodiments, a lipid nanoparticle may include an mRNA encoding a polypeptide of interest capable of being translated within a cell to produce a polypeptide of interest. In other embodiments, the lipid nanoparticle can include other types of agents, such as other nucleic acid agents, including DNA and/or RNA agents, as described herein, e.g., siRNAs, miRNAs, antisense nucleic acid and the like as described in further detail below.

The amount of a nucleic acid molecule in a lipid nanoparticle may depend on the size, composition, desired target and/or application, or other properties of the lipid nanoparticle as well as on the properties of the therapeutic and/or prophylactic. For example, the amount of an RNA useful in a lipid nanoparticle may depend on the size, sequence, and other characteristics of the RNA. The relative amounts of a nucleic acid molecule and other elements (e.g., lipids) in a lipid nanoparticle may also vary. In some embodiments, the wt/wt ratio of the ionizable lipid component to a a nucleic acid molecule, in a lipid nanoparticle may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the ionizable lipid component to a nucleic acid molecule may be from about 10:1 to about 40:1. In certain embodiments, the wt/wt ratio is about 20:1. The amount of a nucleic acid molecule in a LNP may, for example, be measured using absorption spectroscopy (e.g., ultraviolet-visible spectroscopy).

In some embodiments, a lipid nanoparticle includes one or more RNAs, and one or more ionizable lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 5.7:1, about 5.8:1, about 5.9:1, about 6.0:1, about 6.5:1, or about 7.0:1. For example, the N:P ratio may be about 5.67:1. In another embodiment, the N:P ratio may be about 5.8:1.

In some embodiments, the formulation including a lipid nanoparticle may further includes a salt, such as a chloride salt.

In some embodiments, the formulation including a lipid nanoparticle may further includes a sugar such as a disaccharide. In some embodiments, the formulation further includes a sugar but not a salt, such as a chloride salt.

### Physical Properties

The characteristics of a lipid nanoparticle may depend on the components thereof. For example, a lipid nanoparticle including cholesterol as a structural lipid may have different characteristics than a lipid nanoparticle that includes a different structural lipid. Similarly, the characteristics of a lipid nanoparticle may depend on the absolute or relative amounts of its components. For instance, a lipid nanoparticle including a higher molar fraction of a phospholipid may have different characteristics than a lipid nanoparticle including a lower molar fraction of a phospholipid. Characteristics may also vary depending on the method and conditions of preparation of the lipid nanoparticle.

Lipid nanoparticles may be characterized by a variety of methods. For example, microscopy (e.g., transmission electron microscopy or scanning electron microscopy) may be used to examine the morphology and size distribution of a lipid nanoparticle. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a lipid nanoparticle, such as particle size, polydispersity index, and zeta potential.

The mean size of a lipid nanoparticle may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a lipid nanoparticle may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a lipid nanoparticle may be from about 70 nm to about 100 nm. In a particular embodiment, the mean size may be about 80 nm. In other embodiments, the mean size may be about 100 nm.

A lipid nanoparticle may be relatively homogenous. A polydispersity index may be used to indicate the homogeneity of a LNP, e.g., the particle size distribution of the lipid nanoparticles. As used herein, the “polydispersity index” is a ratio that describes the homogeneity of the particle size distribution of a system. A small value, e.g., less than 0.3, indicates a narrow particle size distribution. A small (e.g., less than 0.3) polydispersity index generally indicates a narrow particle size distribution. A lipid nanoparticle may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a lipid nanoparticle may be from about 0.10 to about 0.20.

The zeta potential of a lipid nanoparticle may be used to indicate the electrokinetic potential of the composition. As used herein, the “zeta potential” is the electrokinetic potential of a lipid, e.g., in a particle composition.

For example, the zeta potential may describe the surface charge of a lipid nanoparticle. Lipid nanoparticles with relatively low charges, positive or negative, are generally desirable, as more highly charged species may interact undesirably with cells, tissues, and other elements in the body. In some embodiments, the zeta potential of a lipid nanoparticle may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a a nucleic acid molecule describes the amount of nucleic acid molecule that is encapsulated or otherwise associated with a lipid nanoparticle after preparation, relative to the initial amount provided. The encapsulation efficiency is desirably high (e.g., close to 100%). The encapsulation efficiency may be measured, for example, by comparing the amount of nucleic acid molecule in a solution containing the lipid nanoparticle before and after breaking up the lipid nanoparticle with one or more organic solvents or detergents. Fluorescence may be used to measure the amount of free nucleic acid molecules (e.g., RNA) in a solution. For the lipid nanoparticles described herein, the encapsulation efficiency of a nucleic acid molecule may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A lipid nanoparticle may optionally comprise one or more coatings. For example, a lipid nanoparticle may be formulated in a capsule, film, or tablet having a coating. A capsule, film, or tablet including a composition described herein may have any useful size, tensile strength, hardness, or density.

### Pharmaceutical Composit

Formulations comprising lipid nanoparticles of the disclosure may be formulated in whole or in part as pharmaceutical compositions. Pharmaceutical compositions may include one or more lipid nanoparticles. For example, a pharmaceutical composition may include one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition, except insofar as any conventional excipient or accessory ingredient may be incompatible with one or more components of a LNP in the formulation of the disclosure. An excipient or accessory ingredient may be incompatible with a component of a LNP of the formulation if its combination with the component or LNP may result in any undesirable biological effect or otherwise deleterious effect.

A lipid nanoparticle of the disclosure formulated into a pharmaceutical composition can encapsulate a single nucleic acid or multiple nucleic acids. When encapsulating multiple nucleic acids, the nucleic acids can be of the same type (e.g., all mRNA) or can be of different types (e.g., mRNA and DNA). Furthermore, multiple LNPs can be formulated into the same or separate pharmaceutical compositions. For example, the same or separate pharmaceutical compositions can comprise a first LNP and a second LNP, wherein the first and second LNP encapsulate the same or different nucleic acid molecules, wherein the first and second LNP include na immune cell delivery potentiating lipid as a component. In other embodiments, the same or separate pharmaceutical compositions can comprise a first LNP and a second LNP, wherein the first and second LNP encapsulate the same or different nucleic acid molecules, wherein the first LNP includes a immune cell delivery potentiating lipid as a component and the second LNP lacks a immune cell delivery potentiating lipid.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

Lipid nanoparticles and/or pharmaceutical compositions including one or more lipid nanoparticles may be administered to any patient or subject, including those patients or subjects that may benefit from a therapeutic effect provided by the delivery of a therapeutic and/or prophylactic to one or more particular cells, tissues, organs, or systems or groups thereof, such as the renal system. Although the descriptions provided herein of lipid nanoparticles and pharmaceutical compositions including lipid nanoparticles are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more lipid nanoparticles may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., lipid nanoparticle). The amount of the active ingredient is generally equal to the dosage of the active ingredient which would be administered to a subject and/or a convenient fraction of such a dosage such as, for example, one-half or one-third of such a dosage.

Pharmaceutical compositions may be prepared in a variety of forms suitable for a variety of routes and methods of administration. In one embodiment, such compositions are prepared in liquid form or are lyophylized (e.g., and stored at 4° C. or below freezing). For example, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutics and/or prophylactics, additional agents such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

### Uses of Lipid-Based Compositions

The present disclosure provides improved lipid-based compositions, in particular LNP compositions, with enhanced delivery of nucleic acids to immune cells. The present disclosure is based, at least in part, on the discovery that components of LNPs act as immune cell delivery potentiating lipids that enhance delivery of an encapsulated nucleic acid molecule (e.g., an mRNA) to immune cells, such as lymphoid cells and myeloid cells (e.g., T cells, B cells, monocytes and dendritic cells).

The improved lipid-based compositions of the disclosure, in particular LNPs, are useful for a variety of purposes, both in vitro and in vivo, such as for nucleic acid delivery to immune cells, protein expression in or on immune cells, modulating immune cell (e.g., T cell, B cell, monocyte, and/or dendritic cell) activation or activity and decreasing immune cell responses to reduce autoimmunity (e.g., to tolerize T cells).

In various embodiments, a single immune cell disruptor construct can be used or, alternatively, multiple immune cell disruptor constructs can be used in combination. When used in combination, the mRNA constructs can be coformulated into the same LNP (e.g., as described in Example 10) or, alternatively, separate LNPs can be used for separate mRNA constructs. The particular immune cell disruptor mRNAs to be used can be chosen based on the intended or desired activity/effect in vitro and/or in vivo. For example, for in vivo use in situations where both T cells and B cells may be involved and are desired to be inhibited, a combination of one or more TCDs and one or more BCDs can be used, e.g., coformulated (see e.g., Example 10). Such combination treatments for affecting multiple immune cell types (e.g., T cells, B cells, monocytes and dendritic cells) can be devised based on the various types of immune cell disruptor constucts described herein. Alternatively, in situations where a single type of immune cell is known or thought to mediate a particular activity or disease of interest (e.g., a disorder known to be mediated by T cells), then a single type of immune cell disruptor construct (e.g., TCD) may be chosen for use, although multiple forms of that type of disruptor (e.g., multiple TCDs) can be used in combination.

For in vitro protein expression, the immune cell is contacted with the LNP by incubating the LNP and the immune cell ex vivo. Such immune cells may subsequently be introduced in vivo.

For in vivo protein expression, the immune cell is contacted with the LNP by administering the LNP to a subject to thereby increase or induce protein expression in or on immune cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

For in vitro delivery, in one embodiment the immune cell is contacted with the LNP by incubating the LNP and the immune cell ex vivo. In one embodiment, the immune cell is a human immune cell. In another embodiment, the immune cell is a primate immune cell. In another embodiment, the immune cell is a human or non-human primate immune cell. In one embodiment, the immune cell is a T cell (e.g., a CD3+ T cell, a CD4+ T cell, a CD8+ T cell or a CD4+CD25+CD127^{low} Treg cell). In one embodiment, the immune cell is a B cell (e.g., a CD19+ B cell). In one embodiment, the immune cell is a dendritic cell (e.g., a CD11c+CD11b-dendritic cell). In one embodiment, the immune cell is a monocyte/macrophage (e.g., a CD11c-CD11b+ monocyte/macrophage). In one embodiment, the immune cell is an immature NK cell (e.g., a CD56^{HIGH} immature NK cell). In one embodiment, the immune cell is an activated NK cell (e.g., a CD56^{DIM} activated NK cell). In one embodiment, the immune cell is an NK T cell (e.g., a CD3+CD56+ NK T cell).

In one embodiment, the immune cell is contacted with the LNP in the presence of serum or C1q for at least 15 minutes, which has been shown to be sufficient time for transfection of the cells ex vivo. In another embodiment, the immune cell is contacted with the LNP for, e.g., at least 30 minutes, at least 1 hour, at least 2 hours, at least 3 hours, at least 4 hours, at least 5 hours, at least 6 hours, at least 12 hours or at least 24 hours.

In one embodiment, the immune cell is contacted with the LNP for a single treatment/transfection. In another embodiment, the immune cell is contacted with the LNP for multiple treatments/transfections (e.g., two, three, four or more treatments/transfections of the same cells). Repeat transfection of the same cells has been demonstrated to lead to a dose-related increase in the percentage of cells transfected and in the level of expression of a protein encoded by the transfected nucleic acid without impacting cell viability.

In another embodiment, for in vivo delivery, the immune cell is contacted with the LNP by administering the LNP to a subject to thereby deliver the nucleic acid to immune cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

In one embodiment, an intracellular concentration of the nucleic acid molecule in the immune cell is enhanced. In one embodiment, an activity of the nucleic acid molecule in the immune cell is enhanced. In one embodiment, expression of the nucleic acid molecule in the immune cell is enhanced. In on embodiment, the nucleic acid molecule modulates the activation or activity of the immune cell. In one embodiment, the nucleic acid molecule decreases the activation or activity of the immune cell.

In certain embodiments, delivery of a nucleic acid to an immune cell by the immune cell delivery potentiating lipid-containing LNP results in delivery to a detectable amount of immune cells (e.g., delivery to a certain percentage of immune cells), e.g., in vivo following administration to a subject. In some embodiments, the immune cell delivery potentiating lipid containing LNP does not include a targeting moiety for immune cells (e.g., does not include an antibody with specificity for an immune cell marker, or a receptor ligand which targets the LNP to immune cells). For example, in one embodiment, administration of the immune cell delivery potentiating lipid-containing LNP results in delivery of the nucleic acid to at least about 15% of splenic T cells in vivo after a single intravenous injection. In another embodiment, administration of the immune cell delivery potentiating lipid-containing LNP results in delivery of the nucleic acid to at least about 15%-25% of splenic B cells in vivo after a single intravenous injection. In another embodiment, administration of the immune cell delivery potentiating lipid-containing LNP results in delivery of the nucleic acid to at least about 35%-40% of splenic dendritic cells in vivo after a single intravenous injection. In another embodiment, administration of the immune cell delivery potentiating lipid-containing LNP results in delivery of the nucleic acid to at least about 5%-20% of bone marrow cells (femur and/or humerus) in vivo after a single intravenous injection. The levels of delivery demonstrated herein make in vivo immune therapy possible.

In one embodiment, uptake of the nucleic acid molecule by the immune cell is enhanced. Uptake can be determined by methods known to one of skill in the art. For example, association/binding and/or uptake/internalization may be assessed using a detectably labeled, such as fluorescently labeled, LNP and tracking the location of such LNP in or on immune cells following various periods of incubation. In addition, mathematical models, such as the ordinary differential equation (ODE)-based model described by Radu Mihaila, et al., (Molecular Therapy: Nucleic Acids, Vol. 7: 246-255, 2017; herein incorporated by reference), allow for quantitation of delivery and uptake.

In another embodiment, function or activity of a nucleic acid molecule can be used as an indication of the delivery of the nucleic acid molecule. For example, in the case of mRNA, increase in protein expression in a certain proportion of immune cells can be measured to indicate delivery of the mRNA to that proportion of cells. One of skill in the art will recognize various ways to measure delivery of other nucleic acid molecules to immune cells.

In one embodiment, the activity of the immune disruptor encoded by the nucleic acid molecule in the immune cell is enhanced. In one embodiment, expression of a protein encoded by the nucleic acid molecule in the immune cell is enhanced. In one embodiment, the protein modulates the activation or activity of the immune cell. In one embodiment, the protein decreases the activation or activity of the immune cell.

In one embodiment, various agents can be used to label cells (e.g., T cell, B cell, monocyte, or dendritic cell) to measure delivery to that specific immune cell population. For example, the LNP can encapsulate a reporter nucleic acid (e.g., an mRNA encoding a detectable reporter protein), wherein expression of the reporter nucleic acid results in labeling of the cell population to which the reporter nucleic acid is delivered. Non-limiting examples of detectable reporter proteins include enhanced green fluorescent protein (EGFP) and luciferase.

Delivery of the nucleic acid to the immune cell by the immune cell delivery potentiating lipid-containing LNP can be measured in vitro or in vivo by, for example, detecting expression of a protein encoded by the nucleic acid associated with/encapsulated by the LNP or by detecting an effect (e.g., a biological effect) mediated by the nucleic acid associated with/encapsulated by the LNP. For protein detection, the protein can be, for example, a cell surface protein that is detectable, for example, by immunofluorescence or flow cytometery using an antibody that specifically binds the cell surface protein. Alternatively, a reporter nucleic acid encoding a detectable reporter protein can be used and expression of the reporter protein can be measured by standard methods known in the art.

Methods of the disclosure are useful to deliver nucleic acid molecules to a variety of immune cell types. In one embodiment, the immune cell is selected from the group consisting of T cells, NK cells, dendritic cells and macrophages.

The methods can be used to deliver nucleic acid to immune cells located, for example, in the spleen, in the peripheral blood and/or in the bone marrow. In one embodiment, the immune cell is a T cell. T cells can be identified by expression of one or more T cell markers known in the art, typically CD3. Additional T cell markers include CD4 or CD8. In one embodiment, the immune cell is a B cell. B cells can be identified by expression of one or more B cell markers known in the art, typically CD19. Additional B cell markers include CD24 and CD72. In one embodiment, the immune cell is a monocyte and/or a tissue macrophage. Monocytes and/or macrophages can be identified by expression of one or more monocyte and/or macrophage markers known in the art, such as CD2, CD11b, CD14 and/or CD16. In one embodiment, the immune cell is a dendritic cell. Dendritic cells can be identified by expression of one or more dendritic cell markers known in the art, typically CD11c. Additional dendritic cell markers include BDCA-1 and/or CD103.

The improved lipid-based compositions, including LNPs of the disclosure are useful to deliver more than one nucleic acid molecules to an immune cell or different populations of immune cells, by for example, administration of two or more different LNPs. In one embodiment, the method of the disclosure comprises contacting the immune cell (or administering to a subject), concurrently or consecutively, a first LNP and a second LNP, wherein the first and second LNP encapsulate the same or different nucleic acid molecules, wherein the first and second LNP include a phytosterol as a component. In other embodiments, the method of the disclosure comprises contacting the immune cell (or administering to a subject), concurrently or consecutively, a first LNP and a second LNP, wherein the first and second LNP encapsulate the same or different nucleic acid molecules, wherein the first LNP includes a phytosterol as a component and the second LNP lacks a phytosterol.

### Methods of Inhibiting Immune Cell Activity

The disclosure provides a method for inhibiting immune cell activity (e.g., T cell activity, B cell activity, NK cell activity, dendritic cell activity and/or macrophage activity). In one embodiment, immune cell activity is inhibited in vitro. In another embodiment, immune cell activity is inhibited in vivo, e.g., in a subject, such as a human subject. In one embodiment, the method comprises administering to the immune cell (e.g., administering to a subject) a composition of the disclosure (or lipid nanoparticle thereof, or pharmaceutical composition thereof) comprising at least one polynucleotide (e.g., mRNA) construct encoding an immune cell disruptor (e.g., TCD, BCD), such that activity of the immune cell is inhibited. In one embodiment, inhibiting immune cell activity comprises inhibiting immune cell proliferation. In one embodiment, inhibiting immune cell activity comprises inhibiting cytokine production. In one embodiment, inhibiting immune cell activity comprises inhibiting immunoglobulin production, e.g., antigen-specific antibody production.

Inhibition of immune cell activity, either in vitro or in a subject can be evaluated by a variety of methods established in the art for assessing immune responses, including but not limited to the methods described in the Examples. For example, in various embodiments, inhibition is evaluated by measuring levels of cytokine production and/or antibody production, such as by standard ELISA, and/or by evaluating cell proliferation by standard methods known in the art.

To enhance delivery into an immune cell, polynucleotide compositions of the disclosure can be administered to the immune cell or to a subject encapsulated in a lipid nanoparticle that comprises at least one immune cell delivery potentiating lipid, as described herein. For delivery to B cells in vitro, B cells can be pre-activated as described in Example 6.

Compositions of the disclosure are administered to a subject at an effective amount. In general, an effective amount of the composition will allow for efficient production of the encoded polypeptide in the cell. Metrics for efficiency may include polypeptide translation (indicated by polypeptide expression), level of mRNA degradation, and immune response indicators.

### Therapeutic Methods

The methods of the disclosure for inhibiting immune cell activity in a subject can be used in a variety of clinical, prophylactic or therapeutic applications. For example, the methods can be used to inhibit immune responses (e.g., antigen-specific immune responses) in a subject having aberrant immune activity, including subjects suffering from an autoimmune disease, an allergic disorder or an inflammatory response. Furthermore, the methods can be used to inhibit transplant rejection in organ transplant recipients and inhibit graft-versus-host disease, e.g., in bone marrow transplant recipients. Still further, the methods can be used to downregulate immune cell activity in immunotherapy regimens, to thereby provide control of the degree of immune activation that is stimulated for therapeutic purposes. In particular, in situations where an immunotherapy regimen results in overstimulation of immune responses and detrimental side effects therefrom, the immunoinhibitory methods of the disclosure can be used to “tamp down” the degree of immunostimulation provided by the immunotherapy regimen to thereby lessen detrimental side effects therefrom.

Accordingly, in one aspect, the disclosure pertains to a method of inhibiting an immune response in a subject in need thereof, the method comprising administering to the subject a composition of the disclosure (or lipid nanoparticle thereof, or pharmaceutical composition thereof). The method can further comprise administering one or more additional agents to the subject, such as one or more additional immunoinhibitory or immunosuppressive agents. In some embodiments, the mRNA(s), nanoparticle, or pharmaceutical composition is administered to the patient parenterally. In particular embodiments, the subject is a mammal, e.g., a human. In various embodiments, the subject is provided with an effective amount of the mRNA(s).

Non-limiting examples of autoimmune diseases that can be treated according to the method of the disclosure include rheumatoid arthritis, systemic lupus erythematosus, inflammatory bowel disease (including ulcerative colitis and Crohn's disease), Type 1 diabetes, multiple sclerosis, psoriasis, Graves' disease, Hashimoto's thyroiditis, chronic inflammatory demyelinating polyneuropathy, Guillain-Barre syndrome, myasthenia gravis, glomerulonephritis and vasculitis.

Non-limiting examples of clinical immunotherapy regimens that can be modulated according to the methods of the disclosure include treatment with immune checkpoint inhibitors (e.g., agents that target CTLA4, PD-1 or PD-L1) and treatment with CAR-T cells (adoptive T cell transfer immunotherapies).

A pharmaceutical composition including one or more mRNAs of the disclosure may be administered to a subject by any suitable route. In some embodiments, compositions of the disclosure are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. In some embodiments, a composition is administered intramuscularly. However, the present disclosure encompasses the delivery of compositions of the disclosure by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the patient (e.g., whether the patient is able to tolerate particular routes of administration).

In certain embodiments, compositions of the disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of mRNA or nanoparticle per 1 kg of subject body weight. In particular embodiments, a dose of about 0.005 mg/kg to about 5 mg/kg of mRNA or nanoparticle of the disclosure may be administrated.

In some embodiments the dosage of the RNA polynucleotide in the therapeutic composition is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 100-300 μg, 120-250 μg, 150-250 μg, 180-280 μg, 200-300 μg, 30-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the immunomodulatory therapeutic composition is administered to the subject by intradermal or intramuscular injection. In some embodiments, the immunomodulatory therapeutic composition is administered to the subject on day zero. In some embodiments, a second dose of the immunomodulatory therapeutic composition is administered to the subject on day seven, or day fourteen or day twenty one.

In some embodiments, a dosage of 25 micrograms of the RNA polynucleotide is included in the immunomodulatory therapeutic composition administered to the subject. In some embodiments, a dosage of 10 micrograms of the RNA polynucleotide is included in the immunomodulatory therapeutic composition administered to the subject. In some embodiments, a dosage of 30 micrograms of the RNA polynucleotide is included in the immunomodulatory therapeutic composition administered to the subject. In some embodiments, a dosage of 100 micrograms of the RNA polynucleotide is included in the immunomodulatory therapeutic composition administered to the subject. In some embodiments, a dosage of 50 micrograms of the RNA polynucleotide is included in the immunomodulatory therapeutic composition administered to the subject. In some embodiments, a dosage of 75 micrograms of the RNA polynucleotide is included in the immunomodulatory therapeutic composition administered to the subject. In some embodiments, a dosage of 150 micrograms of the RNA polynucleotide is included in the immunomodulatory therapeutic composition administered to the subject. In some embodiments, a dosage of 400 micrograms of the RNA polynucleotide is included in the immunomodulatory therapeutic composition administered to the subject. In some embodiments, a dosage of 300 micrograms of the RNA polynucleotide is included in the immunomodulatory therapeutic composition administered to the subject. In some embodiments, a dosage of 200 micrograms of the RNA polynucleotide is included in the immunomodulatory therapeutic composition administered to the subject. In some embodiments, the RNA polynucleotide accumulates at a 100 fold higher level in the local lymph node in comparison with the distal lymph node. In other embodiments the immunomodulatory therapeutic composition is chemically modified and in other embodiments the immunomodulatory therapeutic composition is not chemically modified.

In some embodiments, the effective amount is a total dose of 1-100 μg. In some embodiments, the effective amount is a total dose of 100 μg. In some embodiments, the effective amount is a dose of 25 μg administered to the subject a total of one or two times. In some embodiments, the effective amount is a dose of 100 μg administered to the subject a total of two times. In some embodiments, the effective amount is a dose of 1 μg-10 μg, 1 μg-20 μg, 1 μg-30 μg, 5 μg-10 μg, 5 μg-20 μg, 5 μg-30 μg, 5 μg-40 μg, 5 μg-50 μg, 10 μg-15 μg, 10 μg-20 μg, 10 μg-25 μg, 10 μg-30 μg, 10 μg-40 μg, 10 μg-50 μg, 10 μg-60 μg, 15 μg-20 μg, 15 μg-25 μg, 15 μg-30 μg, 15 μg-40 μg, 15 μg-50 μg, 20 μg-25 μg, 20 μg-30 μg, 20 μg-40 μg 20 μg-50 μg, 20 μg-60 μg, 20 μg-70 μg, 20 μg-75 μg, 30 μg-35 μg, 30 μg-40 μg, 30 μg-45 μg 30 μg-50 μg, 30 μg-60 μg, 30 μg-70 μg, 30 μg-75 μg which may be administered to the subject a total of one or two times or more.

A dose may be administered one or more times per day, in the same or a different amount, to obtain a desired level of mRNA expression and/or effect (e.g., a therapeutic effect). The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). For example, in certain embodiments, a composition of the disclosure is administered at least two times wherein the second dose is administered at least one day, or at least 3 days, or least 7 days, or at least 10 days, or at least 14 days, or at least 21 days, or at least 28 days, or at least 35 days, or at least 42 days or at least 48 days after the first dose is administered. In certain embodiments, a first and second dose are administered on days 0 and 2, respectively, or on days 0 and 7 respectively, or on days 0 and 14, respectively, or on days 0 and 21, respectively, or on days 0 and 48, respectively. Additional doses (i.e., third doses, fourth doses, etc.) can be administered on the same or a different schedule on which the first two doses were administered. For example, in some embodiments, the first and second dosages are administered 7 days apart and then one or more additional doses are administered weekly thereafter. In another embodiment, the first and second dosages are administered 7 days apart and then one or more additional doses are administered every two weeks thereafter.

In some embodiments, a single dose may be administered, for example, prior to or after a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

In some embodiments, a pharmaceutical composition of the disclosure may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the disclosure, or imaging, diagnostic, or prophylactic compositions thereof in combination with agents that improve their bioavailability, reduce and/or modify their metabolism, inhibit their excretion, and/or modify their distribution within the body.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

In any of the foregoing or related aspects, the disclosure provides a kit comprising a container comprising a lipid nanoparticle, and an optional pharmaceutically acceptable carrier, or a pharmaceutical composition, and a package insert comprising instructions for administration of the lipid nanoparticle or pharmaceutical composition for inhibiting an immune response in an individual.

In any of the foregoing or related aspects, the disclosure provides a kit comprising a medicament comprising a lipid nanoparticle, and an optional pharmaceutically acceptable carrier, or a pharmaceutical composition, and a package insert comprising instructions for administration of the medicament for inhibiting an immune response in an individual.

### Definitions

An “autoimmune disorder,” as used herein, refers to a disease state in which, via the action of white blood cells (e.g., B cells, T cells, macrophages, monocytes, or dendritic cells), a pathological immune response (e.g., pathological in duration and/or magnitude) against one or more endogenous antigens, i.e., one or more autoantigens, with consequent tissue damage that may result from direct attack on the cells bearing the one or more autoantigens, from immune-complex formation, or from local inflammation. Autoimmune diseases are characterized by increased inflammation due to immune system activation against self-antigens.

The terms “allograft”, “homograft” and “allogeneic graft” refer to the transplant of an organ or tissue from one individual to another of the same species with a different genotype, including transplants from cadaveric, living related, and living unrelated donors. A graft transplanted from one individual to the same individual is referred to as an “autologous graft” or “autograft”. A graft transplanted between two genetically identical or syngeneic individuals is referred to as a “syngeneic graft”. A graft transplanted between individuals of different species is referred to as a “xenogeneic graft” or “xenograft”.

As used herein the phrase “immune response” or its equivalent “immunological response” refers to the development of a cellular (mediated by antigen-specific T cells or their secretion products) directed against an autoantigen or an related epitope of an autoantigen. A cellular immune response is elicited by the presentation of polypeptide epitopes in association with Class I or Class II MHC molecules, to activate antigen-specific CD4+T helper cells and/or CD8+ cytotoxic T cells. The response may also involve activation of other components.

As used herein, the term “immune cell” refers to cells that play a role in the immune response, including lymphocytes, such as B cells and T cells; natural killer cells; myeloid cells, such as monocytes, macrophages, eosinophils, mast cells, basophils, and granulocytes.

An “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

“Immunotherapy” refers to the treatment of a subject afflicted with, or at risk of contracting or suffering a recurrence of, a disease by a method comprising inducing, enhancing, suppressing or otherwise modifying an immune response.

A human “at risk of developing an autoimmune disorder” refers to a human with a family history of autoimmune disorders (e.g., a genetic predisposition to one or more inflammatory disorders) or one exposed to one or more autoimmune disorder/autoantibody-inducing conditions. For example, a human exposed to a shiga toxin is at risk for developing typical HUS. Humans with certain cancers (e.g., liquid tumors such as multiple myeloma or chronic lymphocytic leukemia) can pre-dispose patients to developing certain autoimmune hemolytic diseases. For example, PCH can follow a variety of infections (e.g., syphilis) or neoplasms such as non-Hodgkin's lymphoma. In another example, CAD can be associated with HIV infection, Mycoplasma pneumonia infection, non-Hodgkin's lymphoma, or Waldenstrom's macroglobulinemia. In yet another example, autoimmune hemolytic anemia is a well-known complication of human chronic lymphocytic leukemia, approximately 11% of CLL patients with advanced disease will develop AIHA. As many as 30% of CLL may be at risk for developing AIHA. See, e.g., Diehl et al. (1998) Semin Oncol 25(1):80-97 and Gupta et al. (2002) Leukemia 16(10):2092-2095.

A human “suspected of having an autoimmune disorder” is one who presents with one or more symptoms of an autoimmune disorder. Symptoms of autoimmune disorders can vary in severity and type with the particular autoimmune disorder and include, but are not limited to, redness, swelling (e.g., swollen joints), joints that are warm to the touch, joint pain, stiffness, loss of joint function, fever, chills, fatigue, loss of energy, pain, fever, pallor, icterus, urticarial dermal eruption, hemoglobinuria, hemoglobinemia, and anemia (e.g., severe anemia), headaches, loss of appetite, muscle stiffness, insomnia, itchiness, stuffy nose, sneezing, coughing, one or more neurologic symptoms such as dizziness, seizures, or pain. From the above it will be clear that not all humans are “suspected of having an autoimmune disorder.”

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a LNP, “about” may mean+/−5% of the recited value. For instance, a LNP including a lipid component having about 40% of a given compound may include 30-50% of the compound. In another example, delivery to at least about 15% of T cells may include delivery to 10-20% of T cells.

Cancer: As used herein, “cancer” is a condition involving abnormal and/or unregulated cell growth, e.g., a cell having deregulated control of G1 progression. Exemplary non-limiting cancers include adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, myelodysplastic syndrome (including refractory anemias and refractory cytopenias), myeloproliferative neoplasms or diseases (including polycythemia vera, essential thrombocytosis and primary myelofibrosis), liver cancer (e.g., hepatocellular carcinoma), non-small cell lung cancer, small cell lung cancer, lung carcinoid tumor, lymphoma of the skin, malignant mesothelioma, multiple myeloma, myelodysplasia syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In particular embodiments, the cancer is liver cancer (e.g., hepatocellular carcinoma) or colorectal cancer. In other embodiments, the cancer is a blood-based cancer or a hematopoetic cancer.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering a LNP including the therapeutic and/or prophylactic to the subject (e.g., by an intravenous, intramuscular, intradermal, or subcutaneous route). Administration of a LNP to a mammal or mammalian cell may involve contacting one or more cells with the lipid nanoparticle.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of a LNP, relative to the initial total amount of therapeutic and/or prophylactic used in the preparation of a LNP. For example, if 97 mg of therapeutic and/or prophylactic are encapsulated in a LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Enhanced delivery: As used herein, the term “enhanced delivery” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a nucleic acid (e.g., a therapeutic and/or prophylactic mRNA) by a nanoparticle to a target cell of interest (e.g., immune cell) compared to the level of delivery of the nucleic acid (e.g., a therapeutic and/or prophylactic mRNA) by a control nanoparticle to a target cell of interest (e.g., immune cell). For example, “enhanced delivery” by a immune cell delivery potentiating lipid-containing LNP of the disclosure can be evaluated by comparison to the same LNP lacking an immune cell delivery potentiating lipid. The level of delivery of an immune cell delivery potentiating lipid-containing LNP to a particular cell (e.g., immune cell) may be measured by comparing the amount of protein produced in target cells using the phytoserol-containing LNP versus the same LNP lacking the immune cell delivery potentiating lipid (e.g., by mean fluorescence intensity using flow cytometry), comparing the % of target cells transfected using the immune cell delivery potentiating lipid-containing LNP versus the same LNP lacking the immune cell delivery potentiating lipid (e.g., by quantitative flow cytometry), or comparing the amount of therapeutic and/or prophylactic in target cells in vivo using the immune cell delivery potentiating lipid-containing LNP versus the same LNP lacking the immune cell delivery potentiating lipid. It will be understood that the enhanced delivery of a nanoparticle to a target cell need not be determined in a subject being treated, it may be determined in a surrogate such as an animal model (e.g., a mouse or non-human primate model). For example, for determining enhanced delivery to immune cells, a mouse or NHP model can be used and delivery of an mRNA encoding a protein of interest by a immune cell delivery potentiating lipid-containing LNP can be evaluated in immune cells (e.g., from spleen, peripheral blood and/or bone marrow) (e.g., flow cytometry, fluorescence microscopy and the like) as compared to the same LNP lacking the immune cell delivery potentiating lipid.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a immune cell delivery potentiating lipid in a lipid composition (e.g., LNP) of the disclosure, an effective amount of a immune cell delivery potentiating lipid is an amount sufficient to effect a beneficial or desired result as compared to a lipid composition (e.g., LNP) lacking the immune cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering an immune cell delivery potentiating lipid-containing lipid nanoparticle such that an effective amount of lipid nanoparticles are taken up by immune cells in a subject, an effective amount of immune cell delivery potentiating lipid-containing LNP is an amount sufficient to effect a beneficial or desired result as compared to an LNP lacking the immune cell delivery potentiating lipid. Non-limiting examples of beneficial or desired results in the subject include increasing the percentage of cells transfected, increasing the level of expression of a protein encoded by a nucleic acid associated with/encapsulated by the immune cell delivery potentiating lipid-containing LNP and/or increasing a prophylactic or therapeutic effect in vivo of a nucleic acid, or its encoded protein, associated with/encapsulated by the immune cell delivery potentiating lipid-containing LNP, as compared to an LNP lacking the immune cell delivery potentiating lipid. In some embodiments, a therapeutically effective amount of immune cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of immune cells. For example, an effective amount of immune cell delivery potentiating lipid-containing LNP can be an amount that results in transfection of at least 5%, 10% or 15% of splenic T cells, at least 5%, 10%, 15%, 20% or 25% of splenic B cells and/or at least 5%, 10%, 15%, 20%, 25%, 30%, 35% or 40% of splenic dendritic cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Leaky scanning: A phenomenon known as “leaky scanning” can occur whereby the PIC bypasses the initiation codon and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al., (2017) Nat Med 23(4):501-507).

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Metastasis: As used herein, the term “metastasis” means the process by which cancer spreads from the place at which it first arose as a primary tumor to distant locations in the body. A secondary tumor that arose as a result of this process may be referred to as “a metastasis.”

Modified: As used herein “modified” or “modification” refers to a changed state or a change in composition or structure of a polynucleotide (e.g., mRNA). Polynucleotides may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof).

Modified: As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 mn. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside/Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Pre-Initiation Complex (PIC): As used herein, the term “pre-initiation complex” (alternatively “43S pre-initiation complex”; abbreviated as “PIC”) refers to a ribonucleoprotein complex comprising a 40S ribosomal subunit, eukaryotic initiation factors (eIF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Residence time: As used herein, the term “residence time” refers to the time of occupancy of a pre-initiation complex (PIC) or a ribosome at a discrete position or location along an mRNA molecule.

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian immune cell) compared to an off-target cell (e.g., non-immune cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model). For example, for determining specific delivery to immune cells, a mouse or NHP model (e.g., as described in the Examples) can be used and delivery of an mRNA encoding a protein of interest can be evaluated in immune cells (e.g., from spleen, peripheral blood and/or bone marrow) as compared to non-immune cells by standard methods (e.g., flow cytometry, fluorescence microscopy and the like).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeted cells: As used herein, “targeted cells” refers to any one or more cells of interest. The cells may be found in vitro, in vivo, in situ, or in the tissue or organ of an organism. The organism may be an animal, preferably a mammal, more preferably a human and most preferably a patient. Target immune cells include, for example, CD3+ T cells, CD19+ B cells and CD11c+ dendritic cells, as well as monocytes, tissue macrophages, and bone marrow cells (including immune cells within bone marrow, hematopoietic stem cells, immune cell precursors and fibroblasts).

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning. Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in an sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Preparation of T Cell Disruptor mRNA Constructs

In this example, a series of mRNA constructs were prepared that encoded T cell disruptors (TCDs). Each TCD is a chimeric protein comprising a membrane/signaling complex-associated motif operatively linked to an inhibitory motif. The structure of representative TCDs that were prepared are shown below in Table 17:

TABLE 17
T Cell Disruptor mRNA Constructs

| | | AD | | ID | TCP | TCP |
|---|---|---|---|---|---|---|
| | Association | Amino Acid | Inhibitory | Amino Acid | Nucleotide | Amino Acid |
| TCD# | Domain (AD) | SEQ ID NO: | Domain (ID) | SEQ ID NO: | SEQ ID NO: | SEQ ID NO: |
| TCD1 | hs.ZAP70(nSH2- | 1 | SHP1(PTP) | 21 | 35 | 81 |
| | IA-cSH2-IB) | | | | | |
| TCD2 | hs.ZAP70(nSH2- | 2 | SHP1(PTP) | 21 | 36 | 82 |
| | IA-cSH2-IB.Y/A) | | | | | |
| TCD3 | hs.ZAP70(nSH2- | 3 | SHP1(PTP) | 21 | 37 | 83 |
| | IA-cSH2-GS4) | | | | | |
| TCD4 | hs.ZAP70(nSH2- | 4 | SHP1(PTP) | 21 | 38 | 84 |
| | GS-cSH2-GS4) | | | | | |
| TCD5 | hs.Grb2(SH2) | 5 | SHP1(PTP) | 21 | 39 | 85 |
| TCD6 | hs.Grap(SH2) | 6 | SHP1(PTP) | 21 | 40 | 86 |
| TCD7 | hs.Lck(SH2-SH3) | 7 | SHP1(PTP) | 21 | 41 | 87 |
| TCD8 | hs.LAT(1-160) | 9 | (Y/A/LAIR1.ITIM1) | 22 | 42 | 88 |
| TCD9 | hs.LAT(1-160) | 9 | (Y/A/LAIR1.ITIM2) | 23 | 43 | 89 |
| TCD10 | hs.LAT | 8 | LAIR1(187-287/ITIM) | 24 | 44 | 90 |
| TCD11 | hs.LAT | 8 | SHP1(PTP) | 21 | 45 | 91 |
| TCD12 | hs.LAT(1-38) | 10 | SHP1(PTP) | 21 | 46 | 92 |
| TCD13 | hs.LAT(1-38) | 10 | LAIR(187-287/ITIM) | 24 | 47 | 93 |
| TCD14 | hs.LAT(1-33) | 11 | CA.Csk | 25 | 48 | 94 |
| | | | (W47A/R107K/E154A) | | | |
| TCD15 | hs.PAG(1-47) | 12 | CA.Csk | 25 | 49 | 95 |
| | | | (W47A/R107K/E154A) | | | |
| TCD16 | hs.Lck(1-50) | 13 | CA.Csk | 25 | 50 | 96 |
| | | | (W47A/R107K/E154A) | | | |
| TCD17 | hs.Fyn(1-50) | 14 | CA.Csk | 25 | 51 | 97 |
| | | | (W47A/R107K/E154A) | | | |
| TCD18 | hs.Src(1-10) | 15 | CA.Csk | 25 | 52 | 98 |
| | | | (W47A/R107K/E154A) | | | |
| TCD19 | hs.LAT(1-33) | 11 | Csk(195-449) | 26 | 53 | 99 |
| TCD20 | hs.PAG(1-47) | 12 | Csk(195-449) | 26 | 54 | 100 |
| TCD21 | hs.Lck(1-50) | 13 | Csk(195-449) | 26 | 55 | 101 |
| TCD22 | hs.Fyn(1-50) | 14 | Csk(195-449) | 26 | 56 | 102 |
| TCD23 | hs.Src(1-10) | 15 | Csk(195-449) | 26 | 57 | 103 |
| TCD24 | hs.LAT(1-33) | 11 | SHP1(PTP) | 21 | 58 | 104 |
| TCD25 | hs.PAG(1-47) | 12 | SHP1(PTP) | 21 | 59 | 105 |
| TCD26 | hs.Lck(1-50) | 13 | SHP1(PTP) | 21 | 60 | 106 |
| TCD27 | hs.Fyn(1-50) | 14 | SHP1(PTP) | 21 | 61 | 107 |
| TCD28 | hs.Src(1-10) | 15 | SHP1(PTP) | 21 | 62 | 108 |
| TCD29 | hs.LAT(1-33) | 11 | SHP1(2-515) | 27 | 63 | 109 |
| TCD30 | hs.PAG(1-47) | 12 | SHP1(2-515) | 27 | 64 | 110 |
| TCD31 | hs.Lck(1-50) | 13 | SHP1(2-515) | 27 | 65 | 111 |
| TCD32 | hs.Fyn(1-50) | 14 | SHP1(2-515) | 27 | 66 | 112 |
| TCD33 | hs.Src(1-10) | 15 | SHP1(2-515) | 27 | 67 | 113 |
| TCD34 | mm.LAT(1-38) | 16 | CTLA4(ITIM) | 28 | 68 | 114 |
| TCD35 | hs.LAT(1-38) | 10 | CTLA4(ITIM) | 28 | 69 | 115 |
| TCD36 | hs.LAT(1-38) | 10 | PTPN1(3-277) | 29 | 70 | 116 |
| TCD37 | hs.PI3K.p85 | 17 | PTEN(1-350) | 30 | 71 | 117 |
| | (del.iSH2) | | (K13E/K289E) | | | |
| TCD38 | hs.PI3K.p85 | 17 | SHIP1(111-910) | 31 | 72 | 118 |
| | (del.iSH2) | | | | | |
| TCD39 | hs.PLCg1(SH2/3) | 18 | n.t. SHIP1(111-910) | 31 | 73 | 119 |
| TCD40 | hs.PLCg1(SH2/3) | 18 | c.t. SHIP1(111-910) | 31 | 74 | 120 |
| TCD41 | hs.PLCg1(SH2/3) | 18 | nt. PTEN(1-350) | 30 | 75 | 121 |
| | | | (K13E/K289E) | | | |
| TCD42 | hs.PLCg1(SH2/3) | 18 | c.t. PTEN(1-350) | 30 | 76 | 122 |
| | | | (K13E/K289E) | | | |
| TCD43 | c.t. KRAS | 19 | n.t. PTEN(1-350) | 30 | 77 | 123 |
| | | | (K13E/K289E) | | | |
| TCD44 | c.t. KRAS | 19 | n.t. hs.PTPN22(1-290) | 32 | 78 | 124 |
| TCD45 | c.t. KRAS | 19 | n.t. hs.PTPN22(1-290; | 33 | 79 | 125 |
| | | | S35A) | | | |
| TCD46 | Lck(n72) | 20 | hs.PTPN22(24-289; | 34 | 80 | 126 |
| | | | S35A) | | | |

The mRNA constructs were prepared by standard methods known in the art and typically also encoded an epitope tag (e.g., V5 and/or FLAG) at the N-terminus and/or C-terminus to facilitate detection. Additionally, all constructs contained a Cap 1 5′ Cap (7mG(5′)ppp(5′)NlmpNp), 5′ UTR, 3′ UTR, a poly A tail of 100 nucleotides and were fully modified with 1-methyl-pseudouridine (m1ψ). The amino acid sequences of the association domains used in the constructs are shown in SEQ ID NOs: 1-20. The amino acid sequences of the inhibitory domains used in the constructs are shown in SEQ ID NOs: 21-34. The nucleotide sequences of the coding region of the representative TCD mRNA constructs, without any epitope tag, are shown in SEQ ID NOs: 35-80 and the ORF amino acid sequences of the TCD constructs, without any epitope tag, are shown in SEQ ID NOs: 81-126. An exemplary 5′ UTRs for use in the constructs is shown in SEQ ID NO: 186. An exemplary 3′ UTR for use in the constructs is shown in SEQ ID NO: 187. In certain constructs, the association domain and the inhibitory domain were separated by a linker sequence. An exemplary linker for use in the constructs has the amino acid sequence (GGGGS)ₙ, wherein n=1-4 (SEQ ID NO: 188).

The TCD mRNA constructs were formulated into lipid nanoparticles comprising Compound X/DSPC/cholesterol/beta-sitosterol/PEG DMG at a ratio of 50:10:10:28.5:1.5. Such lipid nanoparticles (LNPs), which contain beta-sitosterol as an immune cell delivery potentiating lipid, are described further in PCT Application No. PCT/US19/15913, filed Jan. 30, 2019, the entire contents of which is expressly incorporated herein by reference.

### Example 2: T Cell Disruptor mRNA Constructs Reduce T Cell Proliferation In Vitro

In a first series of experiments, the ability of the T cell disruptor mRNA constructs to inhibit the proliferation of activated T cells was examined in vitro. Human T cell were enriched from human peripheral blood mononuclear cell using the EasySep™ Human T Cell Enrichment Kit (Stem Cell Technologies). Isolated human T cells were then fluorescently labeled with 5 μM carboxyfluorescein succinimidyl ester (CFSE) by incubating with the label for 6 minutes in a 37° C. waterbath, inverting the tube to mix for 3 minutes and then quenching the labeling with complete RPMI with 10% FCS. Cells were then plated at 1×10⁵ cells/100 μl in a round bottom 96-well plate in complete RPMI media.

T cells were then activated using human T cell activation beads (anti-CD3/CD28/CD2; Miltenyi Biotec; Catalog No. 130-091-441) at various concentrations (0.3-3 μl beads) in 50 μl media. Control wells were treated only with 50 μl complete RPMI media. Lipid nanoparticles (LNPs) encapsulating a TCD mRNA construct were added to the T cells (100 ng LNP/well in 50 μl complete RPMI media supplemented with 1% human serum). Control wells were treated with a control LNP that does not affect T cell proliferation in 50 μl media. The cells were incubated for 72 hours and then stained for cell viability, CD3, CD4 and CD8. The percentages of CSFE-low CD4+ and CSFE-low CD8+ cells were determined as a measure of cell proliferation.

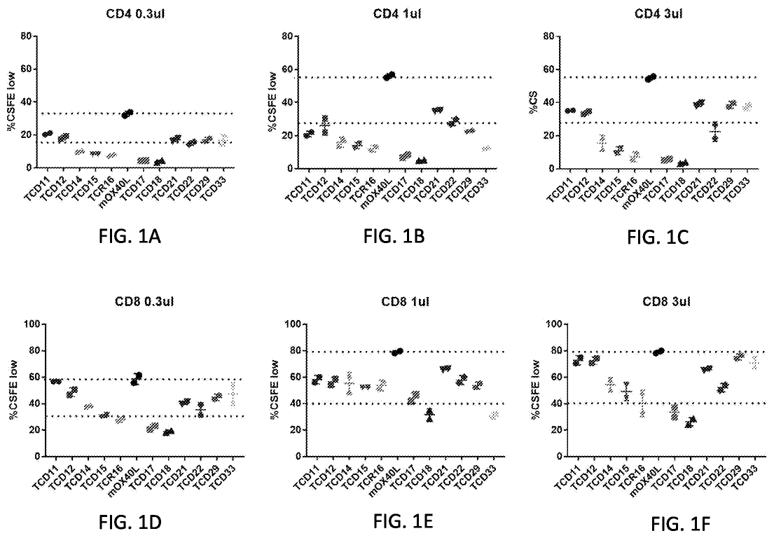

The results are shown in FIGS. 1A-AF, wherein FIGS. 1A-1C show the data for CD4+ cells treated with 0.3 μl, 1.0 μl or 3 μl of activation beads, respectively, and FIGS. 1D-1F show the data for CD8+ cells treated with 0.3 μl, 1.0 μl or 3 μl of activation beads, respectively. The upper dotted line represents the degree of cell proliferation exhibited by T cells treated with the control LNP (i.e., the highest percentage of CSFE-low cells observed), whereas with the lower dotted line represents 50% inhibition of that highest degree of cell proliferation. The results in FIG. 1 demonstrate that the T cell disruptor mRNA constructs reduce proliferation of the activated T cells (both CD4+ and CD8+ cells), even at the higher concentrations of activation beads tested. In particular, many TCD mRNA constructs exhibited over 50% inhibition of T cell proliferation as compared to the control construct.

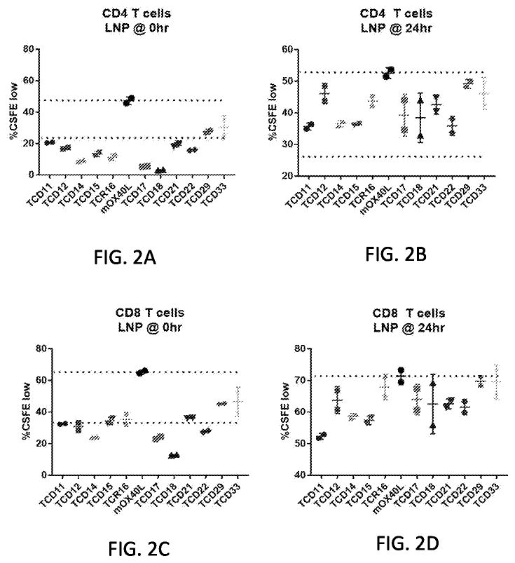

In a second series of experiments, the ability of the TCD mRNA constructs to inhibit proliferation of pre-activated T cells was tested. The proliferation assay was conducted as described above for the first series of experiments, except that the LNPs encapsulating the TCD mRNA constructs were added at either 0 hours or 24 hours post addition of the T cell activation beads. The results are shown in FIGS. 2A-2D, wherein FIGS. 2A-2B show the data for CD4+ cells treated with LNP at either 0 hours or 24 hours post activation, respectively, and FIGS. 2C-2D show the data for CD8+ cells treated with LNP at either 0 hours or 24 hours post activation, respectively. The upper dotted line again represents the degree of cell proliferation exhibited by T cells treated with the control LNP (i.e., the highest percentage of CSFE-low cells observed), whereas with the lower dotted line represents 50% inhibition of that highest degree of cell proliferation. The results in FIGS. 2A-2D demonstrate that the TCD mRNA constructs are able to reduce proliferation of T cells (both CD4+ cells and CD8+ cells) that have been pre-activated for 24 hours.

### Example 3: T Cell Disruptor mRNA Constructs Reduce TNFα Production by T Cells

In this example, the ability of the T cell disruptor mRNA constructs to inhibit the production of TNFα by activated T cells was examined in vitro. Human peripheral blood mononuclear cells (PBMCs) were plated at 1×10⁶ cells/well in a 96-well plate in complete RPMI media. Human PBMC₅ were stimulated with human T cell activation beads (anti-CD3/CD28/CD2; Miltenyi Biotec; Catalog No. 130-091-441) (1.0 μl beads/well) for 24 hours. LNPs encapsulating a TCD mRNA construct were added (100 ng LNP/well in 50 μl complete RPMI media supplemented with 1% human serum) for 24 hours. Control wells were treated with a control LNP that does not affect T cell cytokine production. On the morning of staining, brefeldin A (BFA) (5 μg/mL) and monensin (2.0 μM) were added to the cells 4-5 hours prior to staining. Cells were stained for the CD3, CD4 and CD8 surface markers for 20 minutes at 4 degrees Celsius, followed by fixation and permeabilization of the cells. The cells were then stained for intracellular TNFα by standard methodologies.

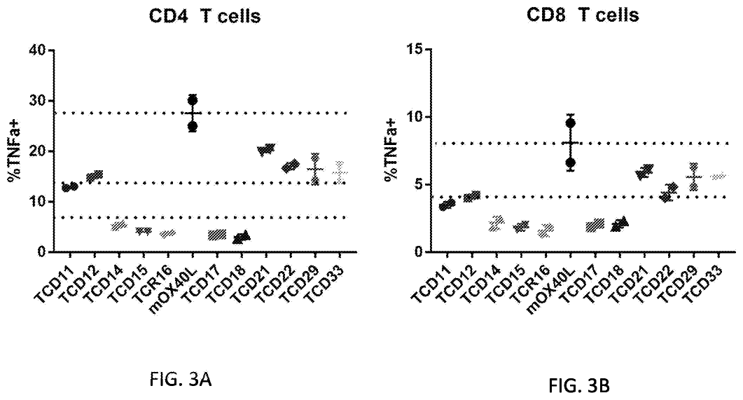

The results are shown in FIGS. 3A-3B, wherein FIG. 3A shows the data for CD4+ T and FIG. 3B show the data for CD8+ T cells. The upper dotted line in each graph represents the percentage of TNFα-positive T cells from the control LNP-treatment (i.e., the highest percentage of TNFα-positive cells observed). For FIG. 3A, the middle and lower dotted lines represent 50% and 75% inhibition, respectively, of the highest degree of TNFα-positive T cells. For FIG. 3B, the lower dotted line represents 50% inhibition of the highest degree of TNFα-positive T cells. The results in FIGS. 3A-3B demonstrate that the TCD mRNA constructs are able to reduce TNFα production in both CD4+ and CD8+ T cells.

### Example 4: T Cell Disruptor mRNA Constructs Inhibit T Cell Activity In Vivo

In this example, a xenogeneic graft versus host disease (xeno-GVHD) animal model was used to examine the effects of the T cell disruptor mRNA constructs in vivo. This animal model system has been described in the art (King et al. (2009) Clin. Exp. Immunol. 157:104-118).

The animals used in the model system are NOD scid gamma (NSG) mice (Jackson Laboratory), which are non-obese diabetic (NOD)-severe combined immunodeficient (scid) ILRrγ^{null} mice that receive gamma irradiation, followed by administration of human peripheral blood mononuclear cells (PMCs) to reconstitute a humanized immune system. Human T cells become activated against mouse antigens, disseminate into peripheral tissues and induce immunopathology leading to weight loss and death. To test the effect of the TCD mRNA constructs on T cell activity in the xeno-GVHD animal model, human PBMC₅ were transfected in vitro overnight with a TCD mRNA construct (1 μg LNP/1×10⁶ cells) or PBS. NSG mice (n=8) were then given 200R irradiation and administered 10×10⁶ transfected human PBMCs intravenously at day 0. On day 3 and day 6, the mice received additional doses of LNP-encapsulated mRNA intravenously (0.5 mg/kg). As a positive control, mice were treated with tacrolimus (TAC) (1.5 mg/kg for the first week, 3.0 mg/mg for the remainder of the experiment subcutaneously). Survival of the mice over time was monitored for 30 days post PBMC injection.

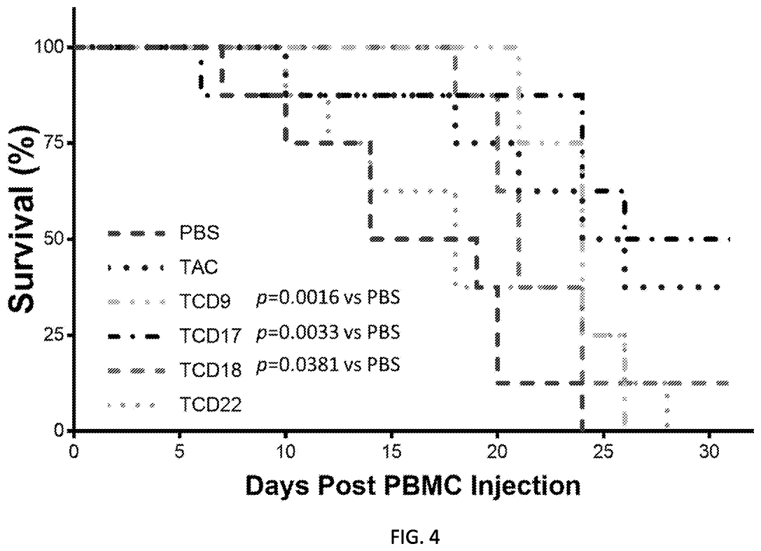

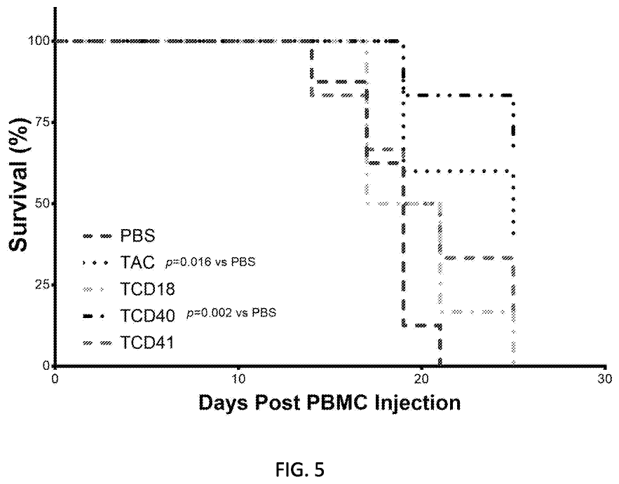

The results from a first series of experiments are shown in FIG. 4. The results from a second series of experiments is shown in FIG. 5. In the second experiment, mice received LNP-encapsulated mRNA weekly at days 7, 14 and 21. These results demonstrate that certain TCD mRNA constructs, in particular TCD #9, TCD #17 and TCD #18 (FIG. 4) and TCD #40 and TCD #41 (FIG. 5), delayed mortality in the xeno-GVHD model, as compared to the PBS negative control treatment group, thereby demonstrating that the TCD mRNA constructs were able to inhibit T cell activity in vivo.

### Example 5: Preparation of B Cell Disruptor mRNA Constructs

In this example, a series of mRNA constructs were prepared that encoded B cell disruptors (BCDs). Each BCD is a chimeric protein comprising a membrane/signaling complex-associated motif operatively linked to an inhibitory motif. The structure of representative BCDs that were prepared are shown below in Table 18:

TABLE 18
B Cell Disruptor mRNA Constructs

| | | AD | | ID | BCD | BCD |
|---|---|---|---|---|---|---|
| | Association | Amino Acid | Inhibitory | Amino Acid | Nucleotide | Amino Acid |
| BCD# | Domain (AD) | SEQ ID NO: | Domain (ID) | SEQ ID NO: | SEQ ID NO: | SEQ ID NO: |
| BCD1 | hCD79a ITAM | 127 | hCD22 ITIM | 144 | 150 | 168 |
| | (Y/A) | | | | | |
| BCD2 | hCD79a (1-176) | 128 | hCD22 ITIM | 144 | 151 | 169 |
| BCD3 | hCD79b ITAM | 129 | hCD22 ITIM | 144 | 152 | 170 |
| | (Y/A) | | | | | |
| BCD4 | hCD79b (1-184) | 130 | hCD22 ITIM | 144 | 153 | 171 |
| BCD5 | hCD19 (ecto-TM) | 131 | hCD22 ITIM | 144 | 154 | 172 |
| BCD6 | hCD19 (ecto-TM) | 131 | hSHP1(PTP) | 145 | 155 | 173 |
| BCD7 | hCD19 (Y/A) | 132 | hCD22 ITIM | 144 | 156 | 174 |
| BCD8 | hCD64 (ecto-TM) | 133 | hCD32b ITIM | 146 | 157 | 175 |
| BCD9 | mCD64 | 134 | mCD32b ITIM | 147 | 158 | 176 |
| | (ecto-TM) | | | | | |
| BCD10 | mCD79a ITAM | 135 | mCD22 ITIM | 148 | 159 | 177 |
| | (Y/A) | | | | | |
| BCD11 | mCD79b ITAM | 136 | mCD22 ITIM | 148 | 160 | 178 |
| | (Y/A) | | | | | |
| BCD12 | mCD19 | 137 | mCD22 ITIM | 148 | 161 | 179 |
| | (ecto-TM) | | | | | |
| BCD13 | mCD19 (Y/A) | 138 | mCD22 ITIM | 148 | 162 | 180 |
| BCD14 | mCD79a | 139 | mCD22 ITIM | 148 | 163 | 181 |
| | (ecto-TM) | | | | | |
| BCD15 | mCD79b | 140 | mCD22 ITIM | 148 | 164 | 182 |
| | (ecto-TM) | | | | | |
| BCD16 | rCD19 (ecto-TM) | 141 | rCD22 ITIM | 149 | 165 | 183 |
| BCD17 | rCD79a | 142 | rCD22 ITIM | 149 | 166 | 184 |
| | (ecto-TM) | | | | | |
| BCD18 | rCD79b | 143 | rCD22 ITIM | 149 | 167 | 185 |
| | (ecto-TM) | | | | | |

The mRNA constructs were prepared by standard methods known in the art and typically also encoded an epitope tag (e.g., V5 and/or FLAG) at the N-terminus and/or C-terminus of the construct (e.g., a FLAG tag at the N-terminus and a V5 tag at the C-terminus) to facilitate detection. Additionally, all constructs typically contained a Cap 1 5′ Cap (7mG(5′)ppp(5′)NlmpNp), 5′ UTR, 3′ UTR, a poly A tail of 100 nucleotides and were fully modified with 1-methyl-pseudouridine (m1ψ). The amino acid sequences of the association domains used in the constructs are shown in SEQ ID NOs: 127-143. The amino acid sequences of the inhibitory domains used in the constructs are shown in SEQ ID NOs: 144-149. The nucleotide sequences of the coding region of the representative BCD mRNA constructs, without any epitope tag, are shown in SEQ ID NOs: 150-167 and the ORF amino acid sequences of the BCD constructs, without any epitope tag, are shown in SEQ ID NOs: 168-185. An xemplary 5′ UTRs for use in the constructs is shown in SEQ ID NO: 186. An exemplary 3′ UTR for use in the constructs is shown in SEQ ID NO: 187. In certain constructs, the association domain and the inhibitory domain were separated by a linker sequence. An exemplary linker for use in the constructs has the amino acid sequence (GGGGS)ₙ, wherein n=1-4 (SEQ ID NO: 188).

The BCD mRNA constructs were formulated into lipid nanoparticles comprising Compound X/DSPC/cholesterol/beta-sitosterol/PEG DMG at a ratio of 50:10:10:28.5:1.5. Such lipid nanoparticles (LNPs), which contain beta-sitosterol as an immune cell delivery potentiating lipid, are described further in PCT Application No. PCT/US19/15913, filed Jan. 30, 2019, the entire contents of which is expressly incorporated herein by reference.

### Example 6: Expression of B Cell Disruptor mRNA Constructs in Vitro

In this example, factors affecting expression of the B cell disruptor (BCD) mRNA constructs in B cells in vitro were examined.

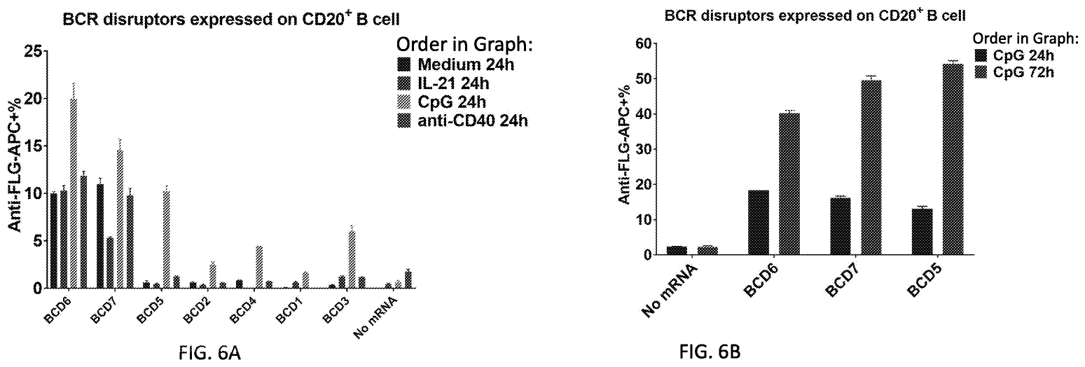

In a first series of experiments, the effect of preactivating B cells was examined. Human PBMC₅ were plated in 96 well plates at 2×10⁵ cells/well and the cells were cocultured with either medium, IL-21 (100 ng/ml), CpG 7909 (5 μg/ml) or anti-CD40 (5 μg/ml) as activating agents, together with 5 μM LNP-encapsulated FLAG-labeled BCD mRNA. The cells were incubated for 24 hours with the activating agents and BCD mRNA, followed by staining with anti-hCD20, anti-hCD14, anti-hCD4, anti-hCD8 and anti-FLAG antibodies and FACS analysis. The results are shown in FIG. 6A, which demonstrates that of the three activating agents tested, CpG preactivated CD20+ B cells exhibited increased expression of mRNA-encoded BCDs as compared to the medium control. To further examine the effect of CpG-mediated preactivation of B cells, human PBMC₅ were cultured as described above for FIG. 6A and CpG (5 μg/ml) was added to the culture for either 24 hours or 72 hours, followed by addition of 5 μM LNP-encapsulated FLAG-labeled BCD mRNA for the last 24 hours. After culturing, the cells again were stained with anti-hCD20, anti-hCD14, anti-hCD4, anti-hCD8 and anti-FLAG antibodies analyzed by FACS analysis. The results are shown in FIG. 6B, which demonstrates that preactivation of B cells with CpG for 72 hours led to even higher levels of expression of the mRNA-encoded BCDs than preactivation for 24 hours.

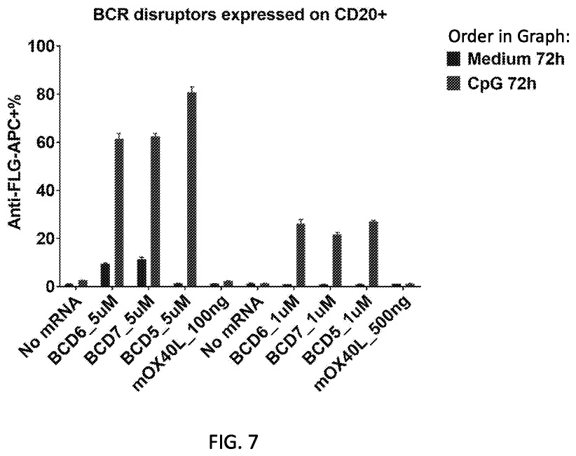

In a second series of experiments, the effect of mRNA concentration on BCD expression was examined. Human PBMC₅ were cultured with either medium or CpG 7909 (5 μg/ml) for 72 hours, followed by addition of LNP-encapsulated BCD mRNA at either 5 μM or 1 μM for the last 24 hours. After culturing, cells were stained with anti-CD20 and anti-FLAG antibodies and analyzed by FACS analysis. The results are shown in FIG. 7, which demonstrates that the BCD mRNA constructs expressed on human B cells show a dose-dependent effect in vitro.

### Example 7: B Cell Disruptor mRNA Constructs Inhibit B Cell Activity In Vitro

In a first series of experiments, the ability of the B cell disruptor mRNA constructs to inhibit the secretion of IgM, IL-6 and IL-10 by B cells was examined in vitro. Human PBMCs were treated with varying concentration of LNP-encapsulated BCD-encoding mRNA (5 μM, 1 μM or 200 nM) and expression levels of hIgM, IL-6 and IL-10 were assayed over 3-5 days. More specifically, human PBMC were seed in 96-well plate at 2×10⁵ cells/well. Cells were stimulated with CpG (as described in Example 6) for 72 hours, and transfected with LNP-encapsulated mRNA for 24 hours, followed by replacement of the culture medium with fresh medium with or without anti-human IgK antibodies for 1-5 days. The supernatants were collected and levels of human IgM, IL-6 and IL-10 were measured by standard ELISA. A mouse OX40L mRNA encapsulated in the same LNP formulation was used as a negative control.

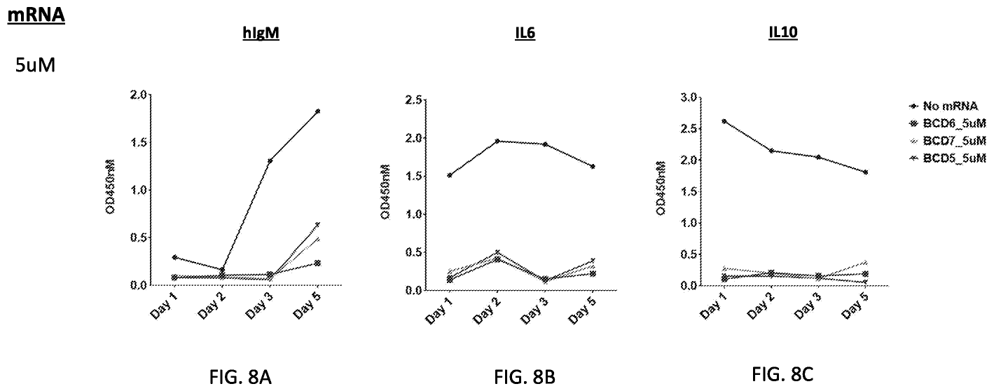

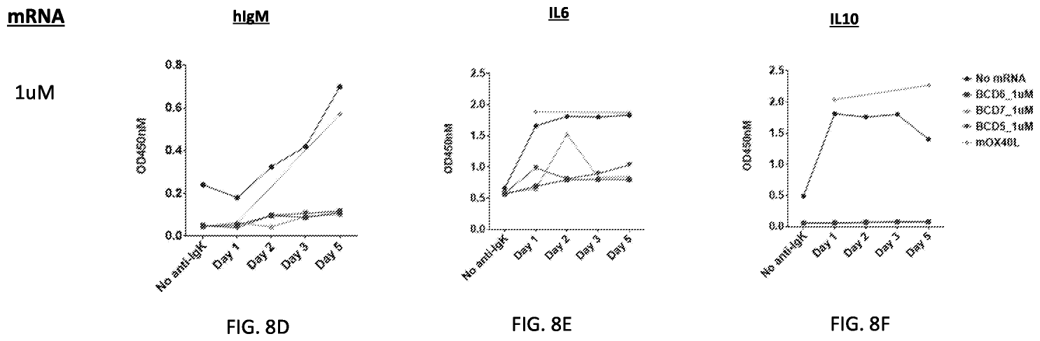

The results are shown in FIGS. 8A-8I, wherein FIGS. 8A-8C show the results for 5 μM mRNA, FIGS. 8D-8F show the results for 1 μM mRNA and FIGS. 8G-8I show the results for 200 nM mRNA. FIGS. 8A, 8D and 8G show the results for hIgM, FIGS. 8B, 8E and 8H show the results for IL-6 and FIGS. 8C, 8F and 8I show the results for IL-10. The results demonstrate that all mRNA constructs tested significantly inhibited hIgM, IL-6 and IL-10 secretion by B cells at the higher concentration tested (1 μM and 5 μM).

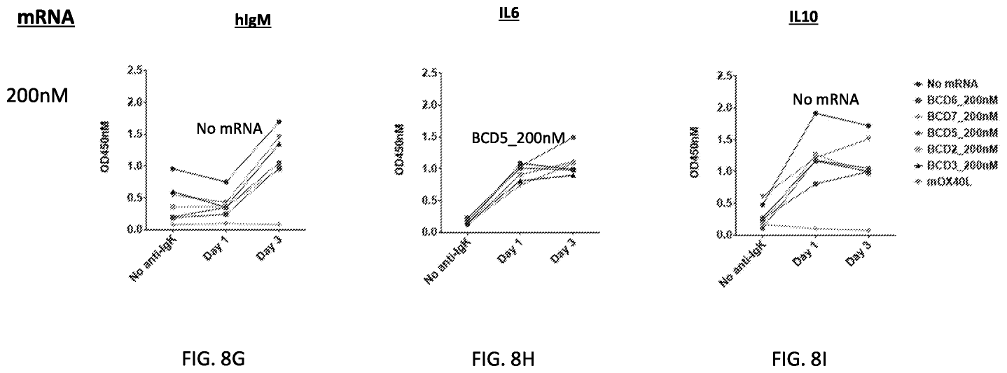

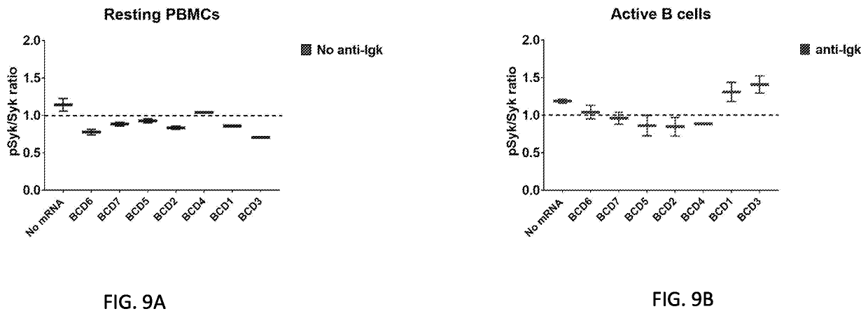

In a second series of experiments, resting PBMC₅ or active B cells were treated with LNP-encapsulated BCD-encoding mRNAs, followed by assaying the level of phosphorylation of Syk. More specifically, human PBMC₅ or isolated human B cells were transfected with LNP-encapsulated mRNA for 24h and the medium was replaced by fresh medium with (FIG. 9A) or without anti-human IgK (FIG. 9B) for 6h or 24h respectively. The cells were lysed and the levels of phosphorylated Syk (p-Syk), Syk and GAPDH were measured by standard ELISA. The reads for pSyk and Syk were normalized to GAPDH.

The results are shown in FIGS. 9A-9B, wherein FIG. 9A shows the ratio of pSyk to Syk for resting PBMC₅ and FIG. 9B shows the ratio of pSyk to Syk for active B cells. The results in FIG. 9A demonstrate that all BCD constructs reduced the level of phosphorylation of Syk on resting PBMCs. The results in FIG. 9B demonstrate that BCD constructs #5 (comprising a truncated form of hCD19), #2 (comprising a truncated form of CD79a) and #4 (comprising a truncated form of CD79b) showed more potent inhibition of Syk phosphorylation in active B cells.

### Example 8: Human B Cell Disruptor mRNA Constructs Inhibit B Cell Activity In Vivo

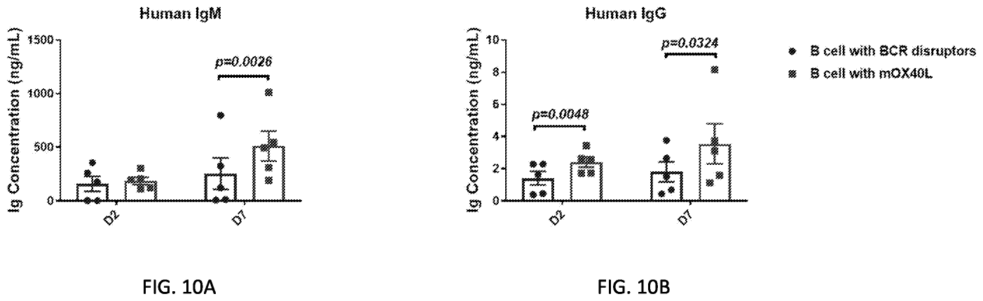

In this example, the NSG animal model described in Example 4 was used to examine the effect of human BCD mRNA constructs on B cell activity in vivo. In a first series of experiments, NSG mice (n=5) were administered 6×10⁶ cells on day 1, wherein 3×10⁶ cells were human B cells transfected ex vivo with mRNA (either BCD-encoding mRNA BCD2, BCD4 or BCDS, or a negative control mRNA) and 3×10⁶ cells were untransfected human T cells from the same donor. On day 2 and day 7, sera was collected and assayed by ELISA for human total IgM and IgG. Also on day 2 and day 7, PBMC and spleen cells were collected and assayed by FACS for mRNA expression and human cell distribution. The mRNA expression and cell distribution analysis revealed that hCD45 cells engrafted into spleen on day 7, that T cell proliferation was observed more in the spleen than in peripheral blood lymphocytes and B cells remained more in peripheral blood lymphocytes than in the spleen. By day 2, BCDs were expressed in 50% of both splenocytes and peripheral blood lymphocytes. On day 7, BCDs were expressed in 38.2% of splenocytes and 16.5% of peripheral blood lymphocytes. The results of the hIgM and hIgG analysis are shown in FIGS. 10A (IgM) and 10B (IgG), which demonstrate that the BCDs reduced both hIgM and hIgG secretion at day 2 and at day 7 in vivo as compared to the negative control mRNA.

In a second series of experiments, the effect of the BCD mRNAs on human B cell recall function was examined in vivo. NSG mice (n=8) were intravenously administered either hPBMC₅ (20×10⁶ cells) or B cells transfected ex vivo with BCD mRNA (5×10⁶ cells) plus untransfected T cells (5×10⁶ cells) on day 1 and tetanus toxoid (15 μg) was administered intraperitoneally on day 2. Whole blood samples were taken on days 4, 7 and 9. Animals were sacrificed on day 15 and spleen and PBMC₅ harvested. The study used five different treatment groups as described in Table 19 below:

TABLE 19
Human B Cell Recall Study Treatment Groups

| Group | | |
|---|---|---|
| # | Cells Injected I.V. | Purpose |
| Gr 1 | B cells transfected with negative | Negative control group |
| | control mRNA + T cells | for mRNA |
| Gr 2 | B cells transfected with B cell | Low dose mRNA expression |
| | disruptor I preparation + T cells | group |
| Gr 3 | B cells transfected with B cell | High dose mRNA expression |
| | disruptor II preparation + T cells | group |
| Gr 4 | hPBMCs + anti-CD20 | Negative control for recall |
| | (10 mg/kg) | study |
| Gr 5 | hPBMCs + PBS | Positive control for recall |
| | | study |

The B cell disruptor I preparation used in Gr 2 contained the following three different BCD mRNA construct: BCD2, BCD4 and BCDS as shown in Table 18. The B cell disruptor II preparation used in Gr 3 contained the following three different BCD mRNA construct: BCD1, BCD3 and BCD7 as shown in Table 18. The B cell disruptor I preparation was used for transfection at a dose of 0.8 mg (“low dose”) and the B cell disruptor II preparation was used for transfection at a dose of 3 mg (“high dose”). FACS analysis of the ex vivo transfected B cells from Gr 2 and Gr 3 confirmed low (20% in CD20+ B cells) and high (50% in CD20+ cells) expression, respectively, of the BCDs.

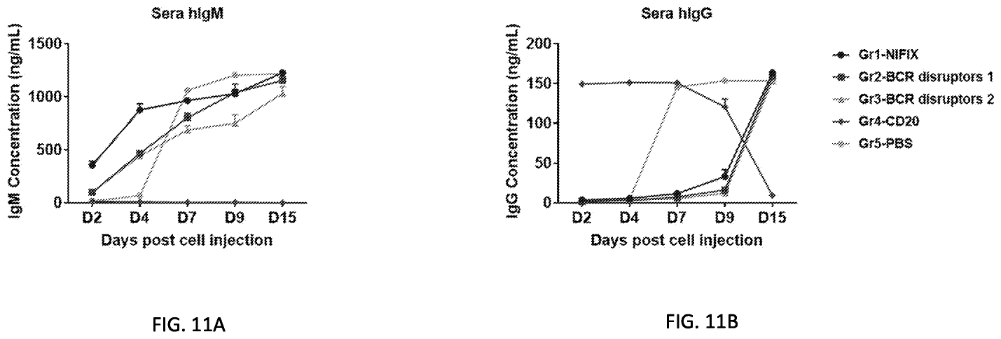

Analysis of the spleens and blood from the five different treatment groups confirmed that both the human PBMC₅ and the ex vivo transfected B cells engrafted into the NSG mice. Splenic enlargement was significant in Gr 5 in which human PBMC₅ were transplanted. Total sera IgM and IgG were measured, the results of which are shown in FIGS. 11A (IgM) and 11B (IgG). The data in FIGS. 11A-11B demonstrated that total sera hIgM and hIgG were increased in both the PBMC-engrafted mice (Gr 5) and the transfected B cell-engrafted mice (Gr 1-3). The ex vivo transfected B cells showed delayed total hIgG secretion. Additionally, compared to the negative control mRNA-transfected B cells (Gr 1), both BCD-transfected B cells (Gr 2 and Gr 3) showed reduced total IgM and IgG, confirming the results observed in the first series of experiments (results shown in FIG. 10).

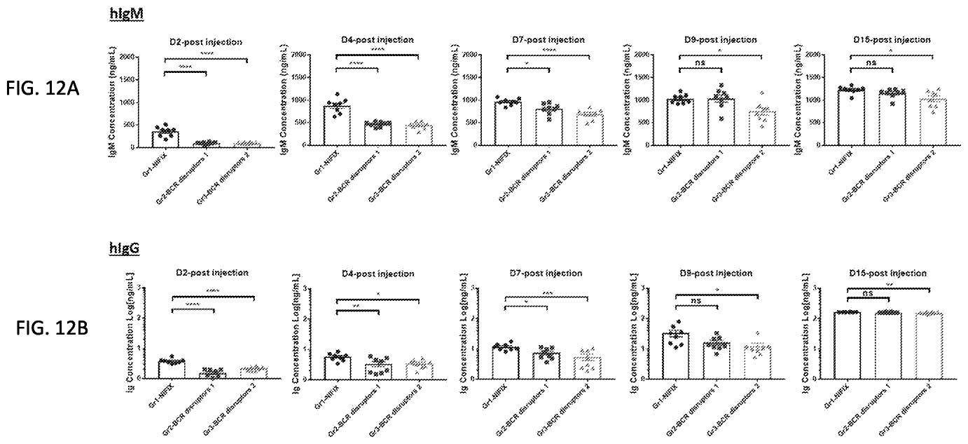

The IgM and IgG concentrations in the control, Gr 2 and Gr 3 mice were monitored on days 2, 4, 7, 9 and 15 post injection, the results of which are shown in FIGS. 12A and 12B, respectively. These results demonstrated that the total hIgM/hIgG suppression in vivo was found to be related to the BCD expression levels on the B cells. In particular, the low-expressed BCD group (Gr 2) showed faster restoration of hIgM/hIgG levels on day 9 post cell injection as compared to the high-expressed BCD group (Gr 3).

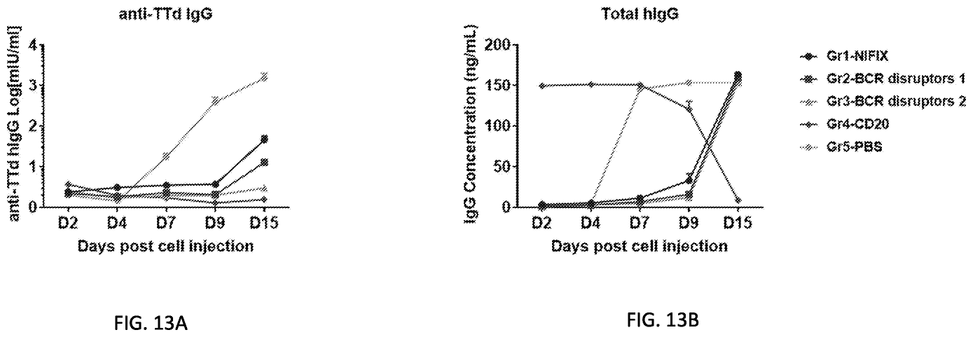

To examine the effect of the BCDs on an antigen-specific antibody response, anti-tetanus toxoid (TTd) antibody levels were measured, the results of which are shown in FIG. 13A, with the total hIgG shown for comparison in FIG. 13B. These results demonstrated that the PBMC-engrafted Gr 5 mice showed a rapid and strong antibody response against TTd from day 7 to day 15, while the anti-CD20 treated B cell depleted Gr 4 mice had no anti-TTd response during day 4 to day 15 post cell injection.

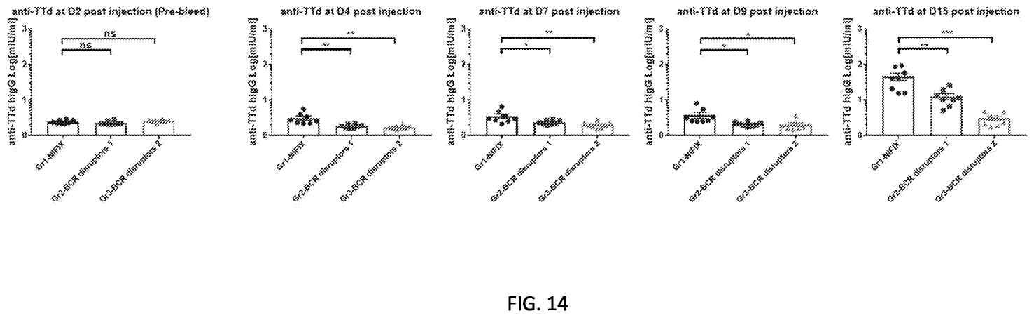

The anti-TTd hIgG concentrations in the control, Gr 2 and Gr 3 mice were monitored on days 2, 4, 7, 9 and 15 post injection, the results of which are shown in FIG. 14. The ex vivo transfected B cell-engrafted mice (Gr 1-3) showed increased anti-TTd hIgG on day 9 post cell injection (day 7 after antigenic stimulation), correlating with the increased total hIgG production observed on day 9 for these treatment groups. Compared to the negative control mRNA-transfected group (Gr 1), both BCD-transfected groups (Gr 2 and Gr 3) showed reduced anti-TTd hIgG on days 9-15 post cell injection, demonstrating that the BCDs were effective in inhibiting antigen-specific antibody accumulation in serum after antigenic challenge. The anti-TTd hIgG suppression observed in vivo was found to be related to the level of expression of the BCDs on the B cells, with the high-expressed BCD group (Gr 3) showing more suppression of anti-TTd hIgG on day 15 post cell injection than the low-expressed BCD group (Gr 2).

### Example 9: In Vitro Studies with Murine B Cell Disruptor mRNA Constructs

In this example, a series of experiments were performed using mRNA constructs encoding murine B cell disruptors. The structures and sequences of murine BCD mRNA constructs are set forth in Table 18 as BCD9, BCD10, BCD11, BCD12, BCD13, BCD14 and BCD15.

Murine BCD mRNA constructs were expressed in resting and activated rat B cells, followed by analysis of IgG secretion, IgM secretion and IL-10 secretion. More specifically, rat splenocytes were seed in 96-well plate at 2×10⁵ cells/well. Cells were stimulated for 48 hours with CpG (as described in Example 6) and transfected for 24 hours with LNP-encapsulated mRNA, followed by replacement of the culture medium with fresh medium with (active B cells) or without (resting B cells) goat anti-rat Ig antibodies for 1-3 days. The supernatants were collected and levels of rat IgM, IgG and IL-10 were measured by standard ELISA. The mouse mRNA constructs were formulated in LNPs as described in Example 5.

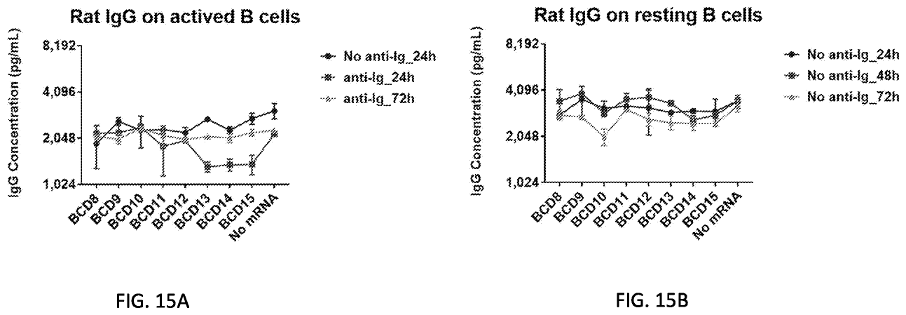

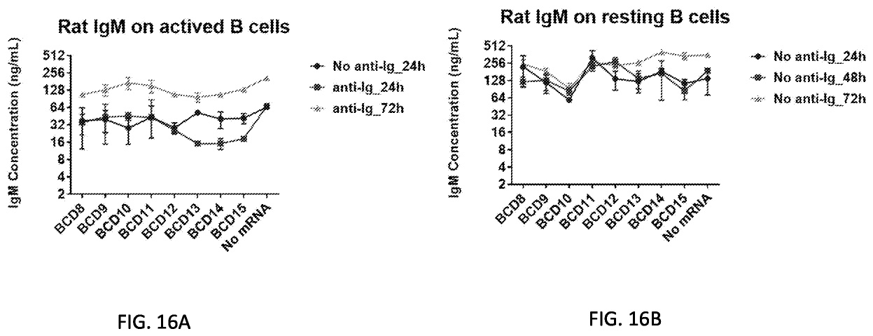

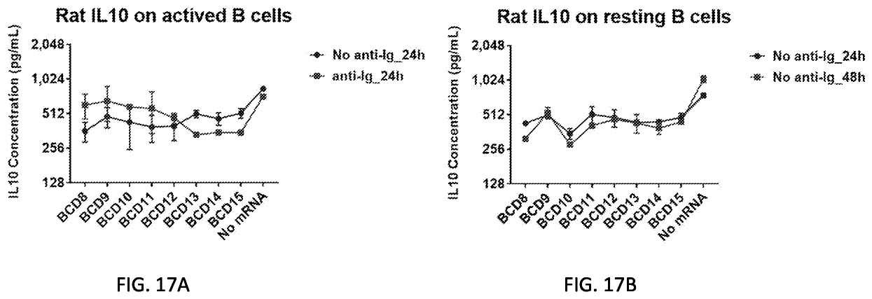

The results for IgG secretion are shown in FIGS. 15A (activated rat B cells) and 15B (resting rat B cells), which demonstrate that the murine BCD constructs reduce IgG secretion on activated rat B cells. The results for IgM secretion are shown in FIGS. 16A (activated rat B cells) and 16B (resting rat B cells), which demonstrate that the murine BCD constructs reduce IgM secretion on activated rat B cells. The results for IL-10 secretion are shown in FIGS. 17A (activated rat B cells) and 17B (resting rat B cells), which demonstrate that the murine BCD constructs reduce IL-10 secretion on activated rat B cells. Thus, these in vitro studies using murine BCD mRNA constructs and rat B cells confirmed the previous results observed with the human BCD mRNA constructs in vitro and in vivo in that the murine BCD mRNA constructs were demonstrated to inhibit B cell activity as measured by either antibody production or cytokine secretion.

### Example 10: Immune Cell Disruptor mRNA Constructs Reduce Autoimmunity In Vivo

In this example, a collagen-induced arthritis (CIA) rat model was used to examine the effect of immune cell disruptor mRNA constructs on immune activity in vivo. In a first series of experiments, Wistar rats (n=3 or 5) were assigned to one of six treatment groups, as follows: (i) naïve (no treatment control); (ii) PBS (negative control); (iii) negative control mRNA (2 mg/kg intravenously); (iv) dexamethasone (5 mg/kg; intraperitoneally); (v) anti-CD20 (10 mg/kg; intraperitoneally); or (vi) immune cell distruptor mRNA (2 mg/kg intravenously).

The immune cell disruptor mRNA constructs TCD18, BCD16, BCD17 and BCD18 were coformulated into a single LNP preparation. The mRNA constructs were formulated into lipid nanoparticles comprising Compound X/DSPC/cholesterol/beta-sitosterol/PEG DMG at a ratio of 50:10:10:28.5:1.5.

On days −7 and 0, rats in groups (ii), (iii) and (v) received their indicated treatments, with blood drawn on day 1 from the anti-CD20-treated group (group (v)) to confirm depletion of CD20+ cells. All rats, except for group (i) (naïve control), were treated on day 1 with collagen type II (CII) in incomplete Freund's adjuvant (IFA). Rats in group (iv) were treated with dexamethasone daily. Rats in group (v) were treated with anti-CD20 on day −7. Rats in group (vi) were treated with the immune cell disruptor mRNAs twice weekly. Blood was collected on days 4, 11, 17 and 21 for serum Ig analysis. On day 21, blood and spleen cells also were collected for FACS and IHC analysis.

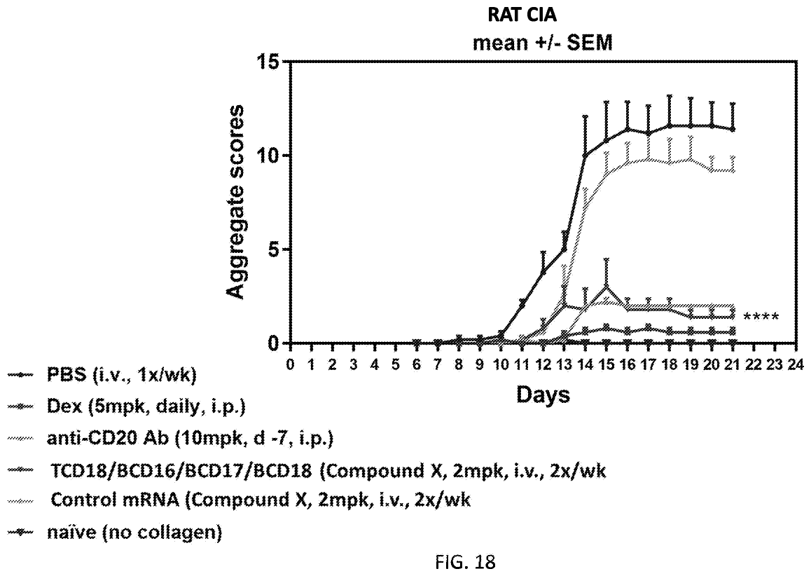

Aggregate scores were determined over time by standard methods, the results of which are shown in FIG. 18. The results demonstrate that, as expected, the naïve rats exhibited no signs of CIA, whereas collagen-induced animals treated with either PBS or the negative control LNP preparation exhibited significant aggregate scores over time. Also as expected, the immune-inhibitory treatments of either dexamethasone or B cell depletion using anti-CD20 led to significantly reduced aggregate scores over time. Finally, collagen-induced animals treated with the immune cell disruptor mRNA constructs also exhibited significantly reduced aggregate scores over time compare to PBS group and negative control mRNA group (p<0.0001), indicating that the mRNA constructs were effective in inhibiting immune activity in vivo in this autoimmune model.

In a second set of experiments, to investigate if the inhibition of CIA development in the immune disruptor-treated rats was due to the lack of an antibody response to type II collagen, the anti-CII-specific levels of IgG in the serum were determined at various time points of the CIA experiment. Rat IgG antibody levels against type II collagen were measured by MSD based enzyme-linked immunosorbent assay (ELISA) methodology using sulfo-tag conjugated secondary goat anti-rat IgG antibody. Serum dilutions of each rat, 1:16000, were chosen after preliminary assays. The optic density was measured using a MESO SECTOR S600 384 plate reader. The anti-type II collagen concentrations were determined by reference to standard curves generated from 1:2 serial dilutions of a standard anti-Collagen II rat antibody (LifeSpan BioSciences, LS-F67398) CIA serum to calculate the antibody content (in arbitrary units/mL).

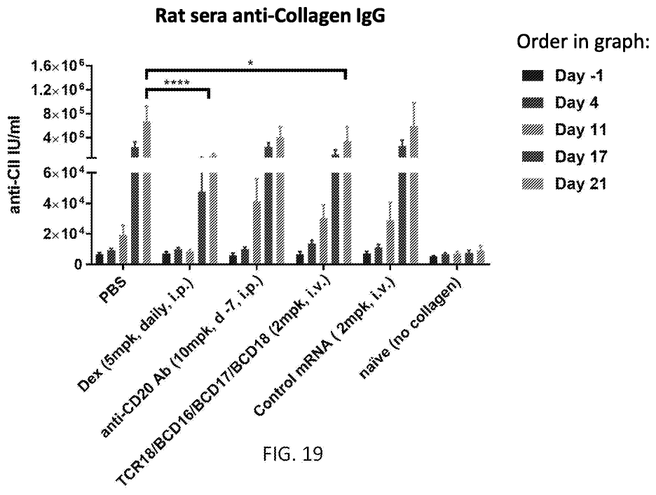

The anti-CII-specific ELISA results are shown in FIG. 19. The results demonstrated that anti-CII antibody levels were significantly lower in the positive control groups (treated with dexamethasone or with anti-CD20 to deplete B cells) and the immune cell disruptor-treated group (p=0.0403) compared to the PBS-treated and control mRNA-treated groups on day 21 after immunization, although no statistically significant difference was observed between the 2 groups on days 4, 11 and 17. These results indicate that over time (e.g., by day 21), treatment with the immune cell disruptor mRNA constructs inhibited antigen-specific antibody production in the CIA model.

### Example 11: Additional B Cell Disruptor mRNA Constructs

In this example, an additional panel of B cell disruptors was prepared, the structures of which are shown below in Table 20:

TABLE 20
Additional B Cell Disruptor mRNA Constructs

| | | AD | | ID | BCD | BCD |
|---|---|---|---|---|---|---|
| | Association | Amino Acid | Inhibitory | Amino Acid | Nucleotide | Amino Acid |
| BCD# | Domain (AD) | SEQ ID NO: | Domain (ID) | SEQ ID NO: | SEQ ID NO: | SEQ ID NO: |
| BCD19 | hSyk(nSH2-IA-cSH2-IB) | 229 | SHP1(PTP) | 145 | 232 | 238 |
| BCD20 | hSyk(nSH2-IA-cSH2-GS) | 230 | SHP1(PTP) | 145 | 233 | 239 |
| BCD21 | hSyk(nSH2-GS-cSH2-GS) | 231 | SHP1(PTP) | 145 | 234 | 240 |
| BCD22 | CD19 | 131 | CA.Csk(W47A/R107K/E154A) | 25 | 235 | 241 |
| BCD23 | hCD79a (1-176) | 128 | CA.Csk(W47A/R107K/E154A) | 25 | 236 | 242 |
| BCD24 | hCD79b (1-184) | 130 | CA.Csk(W47A/R107K/E154A) | 25 | 237 | 243 |

The mRNA constructs were prepared by standard methods known in the art and typically also encoded an epitope tag (e.g., V5 and/or FLAG) at the N-terminus and/or C-terminus of the construct (e.g., a FLAG tag at the N-terminus and a V5 tag at the C-terminus) to facilitate detection. Additionally, all constructs typically contained a Cap 1 5′ Cap (7mG(5′)ppp(5′)NlmpNp), 5′ UTR, 3′ UTR, a poly A tail of 100 nucleotides and were fully modified with 1-methyl-pseudouridine (m1ψ). The amino acid sequences of the association domains used in the constructs are shown in SEQ ID NOs: 128, 130, 131 and 229-231. The amino acid sequences of the inhibitory domains used in the constructs are shown in SEQ ID NOs: 145 and 25. The nucleotide sequences of the coding region of the representative BCD mRNA constructs, without any epitope tag, are shown in SEQ ID NOs: 232-237 and the ORF amino acid sequences of the BCD constructs, without any epitope tag, are shown in SEQ ID NOs: 238-243. An exemplary 5′ UTRs for use in the constructs is shown in SEQ ID NO: 186. An exemplary 3′ UTR for use in the constructs is shown in SEQ ID NO: 187. In certain constructs, the association domain and the inhibitory domain were separated by a linker sequence. An exemplary linker for use in the constructs has the amino acid sequence (GGGGS)ₙ, wherein n=1-4 (SEQ ID NO: 188).

The BCD mRNA constructs were formulated into lipid nanoparticles comprising Compound X/DSPC/cholesterol/beta-sitosterol/PEG DMG at a ratio of 50:10:10:28.5:1.5. Such lipid nanoparticles (LNPs), which contain beta-sitosterol as an immune cell delivery potentiating lipid, are described further in PCT Application No. PCT/US19/15913, filed Jan. 30, 2019, the entire contents of which is expressly incorporated herein by reference.

### Example 12: In Vitro Activity of Additional Immune Cell Disruptor mRNA Constructs

In this example, the additional BCD constructs described in Example 11 were used in in vitro assays to evaluate their immunomodulatory activity, along with BCDS, a negative control mRNA construct and a positive control mRNA construct. In a first set of experiments, Ramos-blue cells were used that carried a secreted alkaline phosphatase (SEAP) reporter gene system that was responsive to B cell receptor cross-linking. Thus, inhibition of SEAP expression was used as an indicator of inhibition of signaling from BCR cross-linking. In these assays, 2×10⁵

Ramos-blue cells were transfected with LNPs comprising BCD constructs or control mRNAs. LNPs were used at 1 μg, 0.5 μg, 0.25 μg and 0.125 μg. Cells were treated with LNPs for 24 hours. The cells were then incubated with fresh media containing anti-IgK (to crosslink BCRs) for 24 hours. The % inhibition of SEAP expression for treated cells was determined, as compared to untreated control cells.

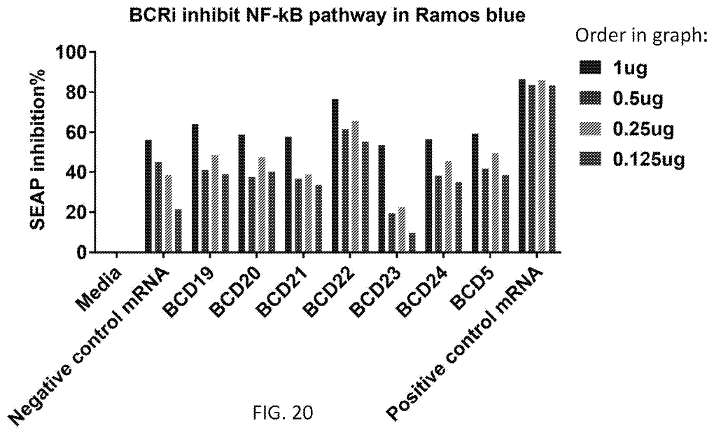

The results are shown in FIG. 20 for all four treatment doses. The SEAP inhibition % for the 0.125 μg dose is summarized below in Table 21:

TABLE 21
SEAP Inhibition % for Additional Immune Disruptor Constructs

| Treatment | SEAP % Inhibition |
|---|---|
| None | 0 |
| Negative control mRNA | 21.5 |
| BCD19 | 38.9 |
| BCD20 | 40.1 |
| BCD21 | 33.6 |
| BCD22 | 55.2 |
| BCD23 | 9.6 |
| BCD24 | 35.0 |
| BCD5 | 38.6 |
| Positive control mRNA | 83.4 |

These results in FIG. 20 and Table 21 confirm that the panel of immune cell disruptor constructs inhibit expression of the SEAP reporter gene in the Ramos-blu cells, thereby indicating the constructs were inhibiting signaling induced by BCR cross-linking.

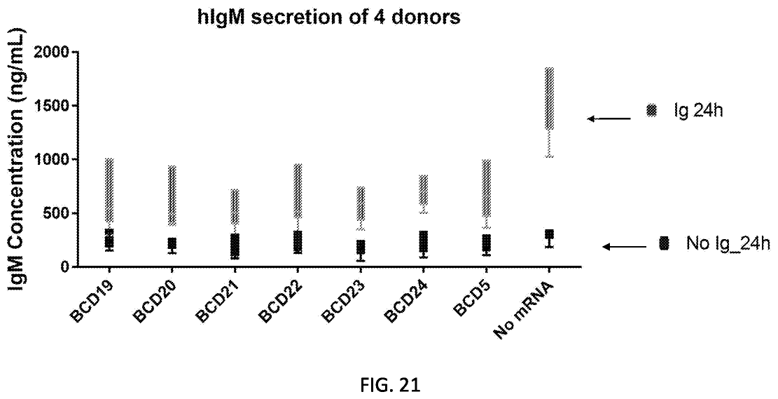

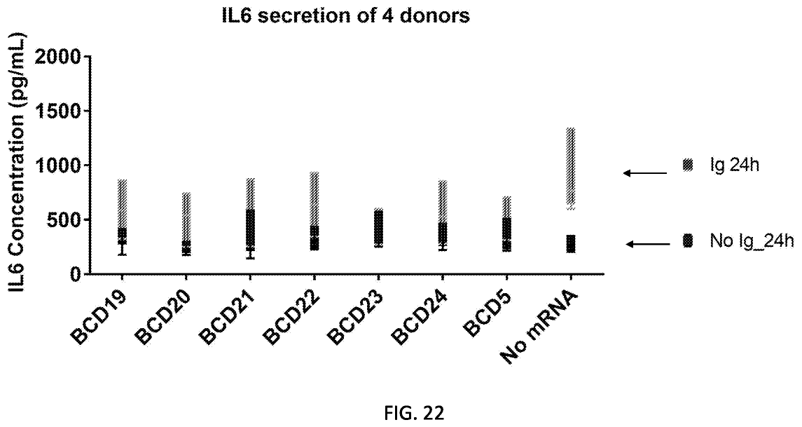

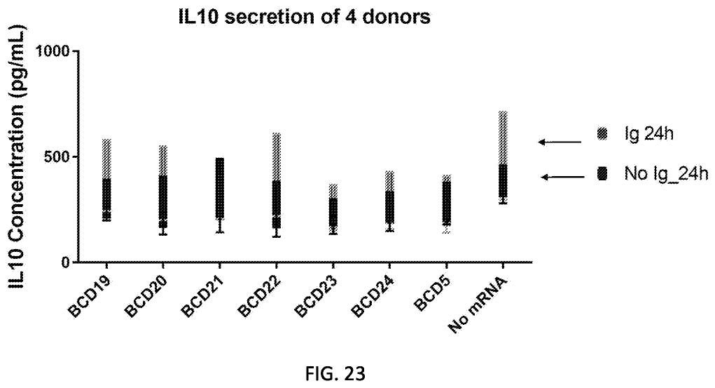

In a second series of experiments, the effect of the constructs on IgM and cytokine (IL-6 and IL-10) secretion by human PBMC₅ was examined. In these assays, 3×10⁵ human PBMCs were stimulated with CpG for 72 hours, then transfected with LNPs comprising the BCD constructs (5 μg/ml) for 24 hours. The cells were then incubated with fresh media with or without anti-IgK (1 μg/ml) (to crosslink BCRs) for 24 hours. Four different PBMC donors were performed separately and each time point was duplicated. The results for IgM secretion, IL-6 secretion and IL-10 secretion are shown in FIGS. 21, 22 and 23, respectively. The results demonstrate that the BCD constructs effectively inhibit secretion of IgM, IL-6 and IL-10 by PBMCs.

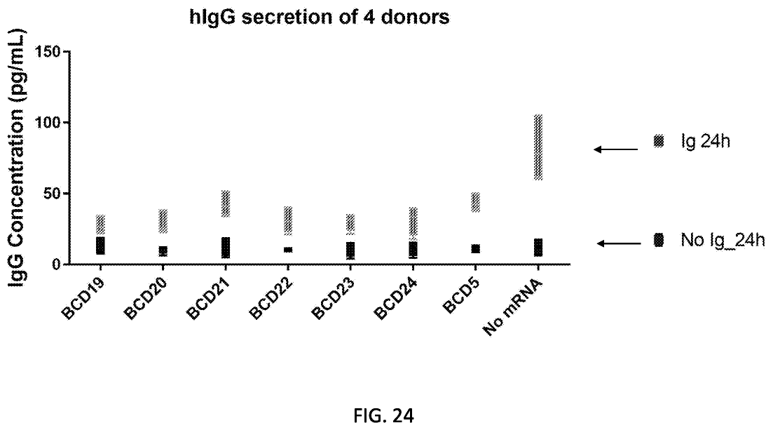

In a third series of experiments, the effect of the constructs on IgG secretion by human PBMC₅ was examined. In these assays, 3×10⁴ human PBMC₅ were cocultured with irradiated EL4B5 feeder cells and stimulated with CpG for 72 hours, then transfected with LNPs comprising the BCD constructs (5 μg/ml) for 24 hours. The cells were then incubated with fresh media with or without anti-IgK (1 μg/ml) (to crosslink BCRs) for 24 hours. Four different PBMC donors were performed separately and each time point was duplicated. The results for IgG secretion are shown in FIG. 24, respectively. The results demonstrate that the BCD constructs also effectively inhibit secretion of IgG by PBMCs.

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

All references described herein are incorporated by reference in their entireties.

SEQUENCE LISTING SUMMARY

| SEQ | |
|---|---|
| ID | |
| NO: | SEQUENCE |
| 1 | MPDPAAHLPFFYGSISRAEAEEHLKLAGMADGLFLLRQCLRSLGGYVLSLVHDVRFHHFPIER |
| | QLNGTYAIAGGKAHCGPAELCEFYSRDPDGLPCNLRKPCNRPSGLEPQPGVFDCLRDAMVRD |
| | YVRQTWKLEGEALEQAIISQAPQVEKLIATTAHERMPWYHSSLTREEAERKLYSGAQTDGKFL |
| | LRPRKEQGTYALSLIYGKTVYHYLISQDKAGKYCIPEGTKFDTLWQLVEYLKLKADGLIYCLK |
| | EACPNSSASNASGAAAPTLPAHPSTLTHPQRRIDTLNSDGYTPEPARITSPDKPRPMPMDTSVY |
| | ESPYSDPEELKDKKLFLKRDNL |
| | [hs.ZAP70(nSH2-IA-cSH2-IB)] |
| 2 | MPDPAAHLPFFYGSISRAEAEEHLKLAGMADGLFLLRQCLRSLGGYVLSLVHDVRFHHFPIER |
| | QLNGTYAIAGGKAHCGPAELCEFYSRDPDGLPCNLRKPCNRPSGLEPQPGVFDCLRDAMVRD |
| | YVRQTWKLEGEALEQAIISQAPQVEKLIATTAHERMPWYHSSLTREEAERKLYSGAQTDGKFL |
| | LRPRKEQGTYALSLIYGKTVYHYLISQDKAGKYCIPEGTKFDTLWQLVEYLKLKADGLIYCLK |
| | EACPNSSASNASGAAAPTLPAHPSTLTHPQRRIDTLNSDGATPEPARITSPDKPRPMPMDTSVA |
| | ESPASDPEELKDKKLFLKRDNL |
| | [hs.ZAP70(nSH2-IA-cSH2-IB.Y/A)] |
| 3 | MPDPAAHLPFFYGSISRAEAEEHLKLAGMADGLFLLRQCLRSLGGYVLSLVHDVRFHHFPIER |
| | QLNGTYAIAGGKAHCGPAELCEFYSRDPDGLPCNLRKPCNRPSGLEPQPGVFDCLRDAMVRD |
| | YVRQTWKLEGEALEQAIISQAPQVEKLIATTAHERMPWYHSSLTREEAERKLYSGAQTDGKFL |
| | LRPRKEQGTYALSLIYGKTVYHYLISQDKAGKYCIPEGTKFDTLWQLVEYLKLKADGLIYCLK |
| | EAC |
| | [hs.ZAP70(nSH2-IA-cSH2-GS4)] |
| 4 | MPDPAAHLPFFYGSISRAEAEEHLKLAGMADGLFLLRQCLRSLGGYVLSLVHDVRFHHFPIER |
| | QLNGTYAIAGGKAHCGPAELCEFYSRDPDGLPCNLRKPCGGGGSGGGGSGGGGSGGGGSWY |
| | HSSLTREEAERKLYSGAQTDGKFLLRPRKEQGTYALSLIYGKTVYHYLISQDKAGKYCIPEGT |
| | KFDTLWQLVEYLKLKADGLIYCLKEAC |
| | [hs.ZAP70(nSH2-GS-cSH2-GS4)] |
| 5 | WFFGKIPRAKAEEMLSKQRHDGAFLIRESESAPGDFSLSVKFGNDVQHFKVLRDGAGKYFLW |
| | VVKFNSLNELVDYHRSTSVSRNQQIFLRDIE |
| | [hs.Grb2(SH2)] |
| 6 | MWYSGRISRQLAEEILMKRNHLGAFLIRESESSPGEFSVSVNYGDQVQHFKVLREASGKYFLW |
| | EEKFNSLNELVDFYRTTTIAKKRQIFLRDEEPL |
| | hs.Grap(SH2) |
| 7 | MGCGCSSHPEDDWMENIDVCENCHYPIVPLDGKGTLLIRNGSEVRDPLVTYEGSNPPASPLQD |
| | NLVIALHSYEPSHDGDLGFEKGEQLRILEQSGEWWKAQSLTTGQEGFIPFNFVAKANSLEPEP |
| | WFFKNLSRKDAERQLLAPGNTHGSFLIRESESTAGSFSLSVRDFDQNQGEVVKHYKIRNLDNG |
| | GFYISPRITFPGLHELVRHYTNASDGLCTRLSRPCQTQKPQKPWWEDEWEVPRET |
| | hs.Lck(SH2-SH3) |
| 8 | MEEAILVPCVLGLLLLPILAMLMALCVHCHRLPGSYDSTSSDSLYPRGIQFKRPHTVAPWPPA |
| | YPPVTSYPPLSQPDLLPIPRSPQPLGGSHRTPSSRRDSDGANSVASYENEGASGIRGAQAGWGV |
| | WGPSWTRLTPVSLPPEPACEDADEDEDDYHNPGALVVLPDSTPATSTAAPSAPALSTPGIRDS |
| | AFSMESIDDAVNVPESGESAEASLDGSREAVNVSQELHPGAAKTEPAALSSQEAEEVEEEGAP |
| | DYENLQELN |
| | [hs.LAT] |
| 9 | MEEAILVPCVLGLLLLPILAMLMALCVHCHRLPGSYDSTSSDSLYPRGIQFKRPHTVAPWPPA |
| | YPPVTSYPPLSQPDLLPIPRSPQPLGGSHRTPSSRRDSDGANSVASYENEGASGIRGAQAGWGV |
| | WGPSWTRLTPVSLPPEPACEDADEDEDDYHNPG |
| | [hs.LAT(1-160)] |
| 10 | MEEAILVPCVLGLLLLPILAMLMALCVHCHRLPGSYDS |
| | hs.LAT(1-38) |
| 11 | MEEAILVPCVLGLLLLPILAMLMALCVHCHRLP |
| | [hs.LAT(1-33)] |
| 12 | MGPAGSLLGSGQMQITLWGSLAAVAIFFVITFLIFLCSSCDREKKPR |
| | [hs.PAG(1-47)] |
| 13 | MGCGCSSHPEDDWMENIDVCENCHYPIVPLDGKGTLLIRNGSEVRDPLVT |
| | [hs.Lck(1-50)] |
| 14 | MGCVQCKDKEATKLTEERDGSLNQSSGYRYGTDPTPQHYPSFGVTSIPNY |
| | [hs.Fyn(1-50]) |
| 15 | MGSNKSKPKD |
| | [hs.Src(1-10)] |
| 16 | MEADALSPVGLGLLLLPFLVTLLAALCVRCRELPVSYD |
| | [mm.LAT(1-38)] |
| 17 | MSAEGYQYRALYDYKKEREEDIDLHLGDILTVNKGSLVALGFSDGQEARPEEIGWLNGYNET |
| | TGERGDFPGTYVEYIGRKKISPPTPKPRPPRPLPVAPGSSKTEADVEQQPAPALPPKPPKPTTVA |
| | NNGMNNNMSLQDAEWYWGDISREEVNEKLRDTADGTFLVRDASTKMHGDYTLTLRKGGNN |
| | KLIKIFHRDGKYGFSDPLTFSSVVELINHYRNESLAQYNPKLDVKLLYPVSKYQQDQVVKEDN |
| | IEAVGKKLHEYNTQFQEKSREYDRLYEEYTRTSQEIQMKRTAIEAFNETIKIFEEQCQTQERYS |
| | KEYIEKFKREGNEKEIQRIMHNYDKLKSRISEIIDSRRRLEEDLKKQAAEYREIDKRMNSIKPDL |
| | IQLRKTRDQYLMWLTQKGVRQKKLNEWLGNENTEDQYSLVEDDEDLPHHDEKTWNVGSSN |
| | RNKAENLLRGKRDGTFLVRESSKQGCYACSVVVDGEVKHCVINKTATGYGFAEPYNLYSSLK |
| | ELVLHYQHTSLVQHNDSLNVTLAYPVYAQQRR |
| | [hs.PI3K.p85(del.iSH2)] |
| 18 | WFHGKLGAGRDGRHIAERLLTEYCIETGAPDGSFLVRESETFVGDYTLSFWRNGKVQHCRIHS |
| | RQDAGTPKFFLTDNLVFDSLYDLITHYQQVPLRCNEFEMRLSEPVPQTNAHESKEWYHASLTR |
| | AQAEHMLMRVPRDGAFLVRKRNEPNSYAISFRAEGKIKHCRVQQEGQTVMLGNSEFDSLVDL |
| | ISYYEKHPLYRKMKLRYPINEEALEKIGTAEPDYGALYEGRNPGFYVEANPMPTFKCAVKALF |
| | DYKAQREDELTFIKSAIIQNVEKQEGGWWRGDYGGKKQLWFPSNYVEEMVN |
| | [hs.PLCg1(SH2/3)] |
| 19 | YRLKKISKEEKTPGCVKIKKC |
| | [KRAS] |
| 20 | MGCGCSSHPEDDWMENIDVCENCHYPIVPLDGKGTLLIRNGSEVRDPLVTYEGSNPPASPLQD |
| | NLVIALHSY |
| | [Lck(1-72)] |
| 21 | FWEEFESLQKQEVKNLHQRLEGQRPENKGKNRYKNILPFDHSRVILQGRDSNIPGSDYINANYI |
| | KNIQLLGPDENAKTYIASQGCLEATVNDFWQMAWQENSRVIVMTTREVEKGRNKCVPYWPE |
| | VGMQRAYGPYSVTNCGEHDTTEYKLRTLQVSPLDNGDLIREIWHYQYLSWPDHGVPSEPGGV |
| | LSFLDQINQRQESLPHAGPIIVHCSAGIGRTGTIIVIDMLMENISTKGLDCDIDIQKTIQMVRAQR |
| | SGMVQTEAQYKFIYVAIAQF |
| | [SHP1(PTP)] |
| 22 | VTYAQLLPDSTPATSTAAPSAPALSTPGIRDSAFSMESIDDVTYAQLPESGESAEASLDGSREVT |
| | YAQLSQELHPGAAKTEPAALSSQEAEEVEEEGAPDYENLQELN |
| | [(Y/A/LAIR1.ITIM1)] |
| 23 | ITYAAVLPDSTPATSTAAPSAPALSTPGIRDSAFSMESIDDITYAAVPESGESAEASLDGSREITY |
| | AAVSQELHPGAAKTEPAALSSQEAEEVEEEGAPDYENLQELN |
| | [(Y/A/LAIR1.ITIM2)] |
| 24 | NHRQNQIKQGPPRSKDEEQKPQQRPDLAVDVLERTADKATVNGLPEKDRETDTSALAAGSSQ |
| | EVTYAQLDHWALTQRTARAVSPQSTKPMAESITYAAVARH |
| | [LAIR1(187-287/ITIM)] |
| 25 | MSAIQAAWPSGTECIAKYNFHGTAEQDLPFCKGDVLTIVAVTKDPNAYKAKNKVGREGIIPAN |
| | YVQKREGVKAGTKLSLMPWFHGKITREQAERLLYPPETGLFLVKESTNYPGDYTLCVSCDGK |
| | VEHYRIMYHASKLSIDEEVYFENLMQLVAHYTSDADGLCTRLIKPKVMEGTVAAQDEFYRSG |
| | WALNMKELKLLQTIGKGEFGDVMLGDYRGNKVAVKCIKNDATAQAFLAEASVMTQLRHSN |
| | LVQLLGVIVEEKGGLYIVTEYMAKGSLVDYLRSRGRSVLGGDCLLKFSLDVCEAMEYLEGNN |
| | FVHRDLAARNVLVSEDNVAKVSDFGLTKEASSTQDTGKLPVKWTAPEALREKKFSTKSDVW |
| | SFGILLWEIYSFGRVPYPRIPLKDVVPRVEKGYKMDAPDGCPPAVYEVMKNCWHLDAAMRPS |
| | FLQLREQLEHIKTHELHL |
| | [CA.Csk(W47A/R107K/E154A)] |
| 26 | LKLLQTIGKGEFGDVMLGDYRGNKVAVKCIKNDATAQAFLAEASVMTQLRHSNLVQLLGVI |
| | VEEKGGLYIVTEYMAKGSLVDYLRSRGRSVLGGDCLLKFSLDVCEAMEYLEGNNFVHRDLA |
| | ARNVLVSEDNVAKVSDFGLTKEASSTQDTGKLPVKWTAPEALREKKFSTKSDVWSFGILLWE |
| | IYSFGRVPYPRIPLKDVVPRVEKGYKMDAPDGCPPAVYEVMKNCWHLDAAMRPSFLQLREQ |
| | LEHIKTHELH |
| | [Csk(195-449)] |
| 27 | VRWFHRDLSGLDAETLLKGRGVHGSFLARPSRKNQGDFSLSVRVGDQVTHIRIQNSGDFYDL |
| | YGGEKFATLTELVEYYTQQQGVLQDRDGTIIHLKYPLNCSDPTSERWYHGHMSGGQAETLLQ |
| | AKGEPWTFLVRESLSQPGDFVLSVLSDQPKAGPGSPLRVTHIKVMCEGGRYTVGGLETFDSLT |
| | DLVEHFKKTGIEEASGAFVYLRQPYYATRVNAADIENRVLELNKKQESEDTAKAGFWEEFESL |
| | QKQEVKNLHQRLEGQRPENKGKNRYKNILPFDHSRVILQGRDSNIPGSDYINANYIKNQLLGP |
| | DENAKTYIASQGCLEATVNDFWQMAWQENSRVIVMTTREVEKGRNKCVPYWPEVGMQRAY |
| | GPYSVTNCGEHDTTEYKLRTLQVSPLDNGDLIREIWHYQYLSWPDHGVPSEPGGVLSFLDQIN |
| | QRQESLPHAGPIIVHCSAGIGRTGTIIVIDMLMENISTKGLDCDIDIQKTIQMVRAQRSGMVQTE |
| | AQYKFIYVAIAQF |
| | [SHP1(2-515)] |
| 28 | AVSLSKMLKKRSPLTTGVYVKMPPTEPECEKQFQPYFIPIN |
| | [CTLA4(ITIM)] |
| 29 | GFGGGGSMEKEFEQIDKSGSWAAIYQDIRHEASDFPCRVAKLPKNKNRNRYRDVSPFDHSRIK |
| | LHQEDNDYINASLIKMEEAQRSYILTQGPLPNTCGHFWEMVWEQKSRGVVMLNRVMEKGSL |
| | KCAQYWPQKEEKEMIFEDTNLKLTLISEDIKSYYTVRQLELENLTTQETREILHFHYTTWPDFG |
| | VPESPASFLNFLFKVRESGSLSPEHGPVVVHCSAGIGRSGTFCLADTCLLLMDKRKDPSSVDIK |
| | KVLLEMRKFRMGLIQTADQLRFSYLAVIEGGKPST |
| | [PTPN1(3-277)] |
| 30 | TAIIKEIVSRNERRYQEDGFDLDLTYIYPNIIAMGFPAERLEGVYRNNIDDVVRFLDSKHKNHY |
| | KIYNLCAERHYDTAKFNCRVAQYPFEDHNPPQLELIKPFCEDLDQWLSEDDNHVAAIHCKAG |
| | KGRTGVMICAYLLHRGKFLKAQEALDFYGEVRTRDKKGVTIPSQRRYVYYYSYLLKNHLDY |
| | RPVALLFHKMMFETIPMFSGGTCNPQFVVCQLKVKIYSSNSGPTRREDKFMYFEFPQPLPVCG |
| | DIKVEFFHKQNKMLKKDKMFHFWVNTFFIPGPEETSEEVENGSLCDQEIDSICSIERADNDKEY |
| | LVLTLTKNDLDKANKDKANRYFSPNFKVKLYFTKT |
| | [PTEN(1-350)(K13E/K289E)] |
| 31 | DPEEDTVESVVSPPELPPRNIPLTASSCEAKEVPFSNENPRATETSRPSLSETLFQRLQSMDTSGL |
| | PEEHLKAIQDYLSTQLAQDSEFVKTGSSSLPHLKKLTTLLCKELYGEVIRTLPSLESLQRLFDQQ |
| | LSPGLRPRPQVPGEANPINMVSKLSQLTSLLSSIEDKVKALLHEGPESPHRPSLIPPVTFEVKAES |
| | LGIPQKMQLKVDVESGKLIIKKSKDGSEDKFYSHKKILQLIKSQKFLNKLVILVETEKEKILRKE |
| | YVFADSKKREGFCQLLQQMKNKHSEQPEPDMITIFIGTWNMGNAPPPKKITSWFLSKGQGKT |
| | RDDSADYIPHDIYVIGTQEDPLSEKEWLEILKHSLQEITSVTFKTVAIHTLWNIRIVVLAKPEHE |
| | NRISHICTDNVKTGIANTLGNKGAVGVSFMFNGTSLGFVNSHLTSGSEKKLRRNQNYMNILRF |
| | LALGDKKLSPFNITHRFTHLFWFGDLNYRVDLPTWEALTIIQKIKQQQYADLLSHDQLLTERRE |
| | QKVFLHFEEEEITFAPTYRFERLTRDKYAYTKQKATGMKYNLPSWCDRVLWKSYPLVHVVC |
| | QSYGSTSDIMTSDHSPVFATFEAGVTSQFVSKNGPGTVDSQGQIEFLRCYATLKTKSQTKFYLE |
| | FHSSCLESFVKSQEGENEEGSEGELVVKFGETLPKLKPIISDPEYLLDQHILISIKSSDSDESYGE |
| | GCIALRLEATETQLPIYTPLTHHGELTGHFQGEIKLQTSQGKTREKLYDFVKTERDESSGPKTL |
| | KSLTSHDPMKQWEVTSRAPPCSGSSITEI |
| | [SHIP1(111-910)] |
| 32 | MDQREILQKFLDEAQSKKITKEEFANEFLKLKRQSTKYKADKTYPTTVAEKPKNIKKNRYKDI |
| | LPYDYSRVELSLITSDEDSSYINANFIKGVYGPKAYIATQGPLSTTLLDFWRMIWEYSVLIIVMA |
| | CMEYEMGKKKCERYWAEPGEMQLEFGPFSVSCEAEKRKSDYIIRTLKVKFNSETRTIYQFHY |
| | KNWPDHDVPSSIDPILELIWDVRCYQEDDSVPICIHCSAGCGRTGVICAIDYTWMLLKDGIIPEN |
| | FSVFSLIREMRTQRPSLVQTQEQYELVYNAVLELF |
| | [h.s. PTPN22(N290)] |
| 33 | MDQREILQKFLDEAQSKKITKEEFANEFLKEKRQATKYKADKTYPTTVAEKPKNIKKNRYKDI |
| | LPYDYSRVELSLITSDEDSSYINANFIKGVYGPKAYIATQGPLSTTELDFWRMIWEYSVLIIVMA |
| | CMEYEMGKKKCERYWAEPGEMQLEFGPFSVSCEAEKRKSDYIIRTLKVKFNSETRTIYQFHY |
| | KNWPDHDVPSSIDPILELIWDVRCYQEDDSVPICIHCSAGCGRTGVICAIDYTWMLLKDGIIPEN |
| | FSVFSLIREMRTQRPSLVQTQEQYELVYNAVLELF |
| | [hs.PTPN22(1-290; S35A)] |
| 34 | FANEFLKEKRQATKYKADKTYPTTVAEKPKNIKKNRYKDILPYDYSRVELSLITSDEDSSYINA |
| | NFIKGVYGPKAYIATQGPLSTTLLDFWRMIWEYSVLIIVMACMEYEMGKKKCERYWAEPGE |
| | MQLEFGPFSVSCEAEKRKSDYIIRTEKVKFNSETRTIYQFHYKNWPDHDVPSSIDPILELIWDVR |
| | CYQEDDSVPICIHCSAGCGRTGVICAIDYTWMLLKDGIIPENFSVFSLIREMRTQRPSLVQTQEQ |
| | YELVYNAVLEL |
| | [hs. PTPN22(24-289; S35A)] |
| 35 | AUGCCUGACCCUGCCGCCCACCUGCCUUUCUUCUACGGCAGCAUCAGCAGAGCCGAGGC |
| | CGAGGAGCACCUGAAGCUGGCCGGCAUGGCCGACGGCCUGUUCCUGCUGAGACAGUGC |
| | CUGAGAAGCCUGGGCGGCUACGUGCUGAGCCUGGUGCACGACGUGAGAUUCCACCACU |
| | UCCCUAUCGAGAGACAGCUGAACGGCACCUACGCCAUCGCCGGCGGCAAGGCCCACUGC |
| | GGCCCUGCCGAGCUGUGCGAGUUCUACAGCAGAGAUCCUGACGGCUUGCCUUGCAACC |
| | UGAGAAAGCCGUGUAAUAGACCUAGCGGCCUGGAGCCUCAGCCUGGCGUGUUCGACUG |
| | UCUGCGCGACGCCAUGGUGAGAGACUACGUGAGACAGACCUGGAAGCUGGAGGGCGAG |
| | GCCCUGGAGCAGGCCAUCAUCAGCCAGGCCCCUCAGGUGGAGAAGCUGAUCGCCACCAC |
| | CGCCCACGAGAGAAUGCCUUGGUACCACAGCAGCCUGACCAGAGAGGAAGCCGAGAGA |
| | AAGCUGUACAGCGGCGCCCAGACCGACGGCAAGUUCUUGCUUCGGCCGAGAAAGGAGC |
| | AGGGCACUUACGCGCUCAGUUUGAUCUACGGAAAGACCGUGUACCACUACCUGAUUUC |
| | UCAGGACAAGGCCGGCAAGUACUGCAUCCCUGAGGGCACCAAGUUCGACACCCUGUGG |
| | CAGCUGGUGGAGUAUCUGAAGCUUAAGGCUGACGGACUGAUCUACUGCCUGAAGGAGG |
| | CCUGCCCUAACAGCAGCGCCAGCAACGCCAGUGGUGCAGCCGCCCCUACCCUGCCUGCC |
| | CACCCUAGCACCCUGACCCACCCUCAGAGAAGAAUUGACACACUGAACAGCGACGGCUA |
| | CACACCAGAGCCUGCCAGAAUCACCAGCCCUGACAAGCCUAGACCUAUGCCUAUGGACA |
| | CCAGCGUGUACGAGAGCCCUUACAGCGACCCUGAGGAGUUAAAGGACAAGAAGUUAUU |
| | CUUGAAGAGAGACAACCUGUUCUGGGAGGAGUUCGAGUCGCUGCAGAAGCAGGAGGUG |
| | AAGAACCUGCACCAGAGACUGGAGGGACAACGGCCAGAGAACAAGGGCAAGAACAGAU |
| | ACAAGAACAUCUUGCCAUUCGAUCACAGCCGGGUGAUCCUGCAGGGCAGGGAUUCCAA |
| | CAUCCCUGGCAGCGACUACAUCAACGCCAAUUACAUUAAGAACCAGCUGCUGGGCCCU |
| | GACGAGAACGCCAAGACCUACAUCGCCAGCCAGGGCUGCCUGGAGGCCACCGUGAACG |
| | ACUUCUGGCAGAUGGCCUGGCAGGAGAAUUCUAGGGUGAUCGUGAUGACCACCCGCGA |
| | AGUGGAGAAGGGCAGAAACAAGUGCGUGCCUUACUGGCCUGAGGUGGGCAUGCAGAGA |
| | GCCUACGGCCCAUACAGCGUGACCAACUGCGGCGAGCACGACACCACCGAGUACAAGCU |
| | GAGAACCCUGCAGGUGAGCCCUUUGGAUAACGGCGACCUGAUCAGAGAGAUCUGGCAC |
| | UACCAGUACCUGUCUUGGCCUGAUCACGGCGUGCCUAGCGAACCAGGCGGCGUGCUAU |
| | CCUUCCUGGACCAGAUCAACCAACGACAGGAGUCCCUACCUCACGCCGGCCCUAUCAUC |
| | GUGCACUGCAGCGCCGGCAUCGGCAGAACCGGCACCAUUAUUGUGAUCGACAUGCUCA |
| | UGGAGAACAUCAGCACCAAGGGCCUGGACUGCGACAUCGACAUCCAGAAGACCAUCCA |
| | GAUGGUGCGGGCUCAGCGCAGCGGCAUGGUGCAGACCGAGGCCCAGUACAAGUUCAUC |
| | UACGUGGCCAUCGCACAGUUC |
| | [TCD1 nt. seq.] |
| 36 | AUGCCUGACCCUGCCGCCCACCUGCCUUUCUUCUACGGCAGCAUCAGCAGAGCCGAGGC |
| | CGAGGAGCACCUGAAGCUGGCCGGCAUGGCCGACGGCCUGUUCCUGCUGAGACAGUGC |
| | CUGAGAAGCCUGGGCGGCUACGUGCUGAGCCUGGUGCACGACGUGAGAUUCCACCACU |
| | UCCCUAUCGAGAGACAGCUGAACGGCACCUACGCCAUCGCCGGCGGCAAGGCCCACUGC |
| | GGCCCUGCCGAGCUGUGCGAGUUCUACAGCCGGGACCCUGACGGCCUCCCUUGCAACCU |
| | GAGAAAGCCUUGUAACAGACCUAGCGGCCUGGAGCCUCAGCCUGGCGUGUUCGACUGU |
| | CUGCGCGACGCCAUGGUGAGAGACUACGUGAGACAGACCUGGAAGCUGGAGGGCGAGG |
| | CCCUGGAGCAGGCCAUCAUCAGCCAGGCCCCUCAGGUGGAGAAGCUGAUCGCCACCACC |
| | GCCCACGAGAGAAUGCCUUGGUACCACAGCAGCCUGACCAGAGAGGAAGCCGAAAGAA |
| | AGCUGUACAGCGGCGCCCAGACCGACGGCAAGUUCUUGCUGAGGCCUAGAAAGGAGCA |
| | GGGUACAUACGCACUUUCCCUGAUUUACGGCAAGACCGUGUACCACUACCUGAUUUCC |
| | CAAGACAAGGCCGGCAAGUACUGCAUCCCUGAGGGCACCAAGUUCGACACCCUGUGGC |
| | AGCUGGUGGAGUAUUUAAAGCUCAAGGCCGACGGCCUAAUCUAUUGUCUCAAGGAGGC |
| | CUGCCCUAACAGCAGCGCCAGCAACGCUAGCGGAGCCGCCGCACCUACCCUGCCUGCCC |
| | ACCCUAGCACCCUGACCCACCCUCAGAGAAGAAUCGAUACUUUGAACAGCGACGGCGCC |
| | ACGCCAGAACCAGCCAGAAUCACCAGCCCUGACAAGCCUAGACCUAUGCCUAUGGACAC |
| | CAGCGUCGCGGAAAGCCCUGCCAGCGACCCUGAGGAACUCAAGGACAAGAAGUUAUUC |
| | CUAAAGAGAGACAACCUGUUCUGGGAGGAGUUCGAGAGCCUGCAGAAGCAGGAGGUGA |
| | AGAACCUGCACCAGAGACUUGAGGGUCAGAGGCCUGAGAACAAGGGCAAGAACAGAUA |
| | CAAGAACAUUCUGCCAUUCGAUCACUCUAGGGUGAUCCUGCAGGGCAGAGACUCUAAC |
| | AUCCCUGGCAGCGACUACAUCAACGCCAACUAUAUAAAGAACCAACUGCUGGGCCCUG |
| | ACGAGAACGCCAAGACCUACAUCGCCAGCCAGGGCUGCCUGGAGGCCACCGUGAACGA |
| | CUUCUGGCAGAUGGCCUGGCAGGAGAAUAGCCGAGUCAUUGUGAUGACUACCAGAGAA |
| | GUCGAGAAGGGCAGAAACAAGUGCGUGCCUUACUGGCCUGAGGUGGGCAUGCAGAGAG |
| | CCUACGGCCCUUACAGCGUGACGAAUUGCGGCGAGCACGACACCACCGAGUACAAGCU |
| | GAGAACCCUGCAGGUGAGCCCUCUGGACAACGGCGAUCUUAUCAGAGAGAUCUGGCAC |
| | UACCAGUAUCUGUCCUGGCCAGAUCACGGCGUGCCUAGCGAGCCAGGCGGCGUUUUGU |
| | CAUUCCUGGACCAGAUCAAUCAGCGACAGGAGAGCUUGCCUCACGCCGGCCCUAUCAU |
| | CGUGCACUGCAGCGCCGGCAUCGGCAGAACCGGCACCAUUAUUGUGAUCGACAUGCUG |
| | AUGGAGAACAUCAGCACCAAGGGCCUGGACUGCGACAUCGACAUCCAGAAGACCAUCC |
| | AGAUGGUGCGAGCUCAGCGCAGCGGCAUGGUGCAGACCGAGGCCCAGUACAAGUUCAU |
| | CUACGUGGCUAUUGCGCAGUUC |
| | [TCD2 nt. seq.] |
| 37 | AUGCCUGACCCUGCCGCCCACCUGCCUUUCUUCUACGGCAGCAUCAGCAGAGCCGAGGC |
| | CGAGGAGCACCUGAAGCUGGCCGGCAUGGCCGACGGCCUGUUCCUGCUGAGACAGUGC |
| | CUGAGAAGCCUGGGCGGCUACGUGCUGAGCCUGGUGCACGACGUGAGAUUCCACCACU |
| | UCCCUAUCGAGAGACAGCUGAACGGCACCUACGCCAUCGCCGGCGGCAAGGCCCACUGC |
| | GGCCCUGCCGAGCUGUGCGAGUUCUACAGCCGGGACCCUGACGGACUUCCUUGCAACC |
| | UGAGAAAGCCUUGUAAUAGACCUAGCGGCCUGGAGCCUCAGCCUGGCGUGUUCGACUG |
| | UUUACGAGACGCCAUGGUGAGAGACUACGUGAGACAGACCUGGAAGCUGGAGGGCGAG |
| | GCCCUGGAGCAGGCCAUCAUCAGCCAGGCCCCUCAGGUGGAGAAGCUGAUCGCCACCAC |
| | CGCCCACGAGAGAAUGCCUUGGUACCACAGCAGCCUGACCAGAGAGGAAGCCGAGAGA |
| | AAGCUGUACAGCGGCGCCCAGACCGACGGCAAGUUCCUCCUGCGGCCUAGAAAGGAGC |
| | AGGGAACAUACGCGUUGUCACUAAUCUACGGCAAGACCGUGUACCACUACCUGAUUUC |
| | CCAGGACAAGGCCGGCAAGUACUGCAUCCCUGAGGGCACCAAGUUCGACACCCUGUGG |
| | CAGCUGGUGGAGUAUUUGAAGCUGAAGGCAGACGGAUUGAUCUACUGCCUCAAGGAGG |
| | CCUGCGGCGGAGGAGGAUCAGGAGGCGGUGGCAGUUUCUGGGAGGAGUUCGAGAGCCU |
| | GCAGAAGCAGGAGGUGAAGAACCUGCACCAGAGAUUGGAGGGACAGAGGCCAGAGAAC |
| | AAGGGCAAGAACAGAUACAAGAACAUUCUUCCUUUCGAUCACAGUCGGGUGAUCCUGC |
| | AGGGCAGAGACUCUAACAUCCCUGGCAGCGACUACAUCAACGCCAAUUACAUUAAGAA |
| | CCAGCUGCUGGGCCCUGACGAGAACGCCAAGACCUACAUCGCCAGCCAGGGCUGCCUGG |
| | AGGCCACCGUGAACGACUUCUGGCAGAUGGCCUGGCAGGAGAACUCAAGAGUAAUUGU |
| | GAUGACCACAAGGGAAGUCGAGAAGGGCAGAAACAAGUGCGUGCCUUACUGGCCUGAG |
| | GUGGGCAUGCAGAGAGCCUACGGCCCUUACAGCGUGACCAACUGCGGCGAGCACGACA |
| | CCACCGAGUACAAGCUGAGAACCCUGCAGGUGAGCCCUCUGGACAACGGCGACUUGAU |
| | UAGAGAGAUCUGGCACUACCAGUAUCUGAGUUGGCCAGACCACGGCGUGCCUAGCGAA |
| | CCUGGUGGCGUCCUUAGUUUCCUGGACCAGAUCAACCAGAGGCAGGAGUCCCUGCCUC |
| | ACGCCGGCCCUAUCAUCGUGCACUGCAGCGCCGGCAUCGGCAGAACCGGCACCAUUAUU |
| | GUGAUCGACAUGCUGAUGGAGAACAUCAGCACCAAGGGCCUGGACUGCGACAUCGACA |
| | UCCAGAAGACCAUCCAGAUGGUUAGGGCCCAGAGAAGCGGCAUGGUGCAGACCGAGGC |
| | CCAGUACAAGUUCAUCUACGUGGCAAUCGCCCAGUUC |
| | [TCD3 nt. seq.] |
| 38 | AUGCCUGACCCUGCCGCCCACCUGCCUUUCUUCUACGGCAGCAUCAGCAGAGCCGAGGC |
| | CGAGGAGCACCUGAAGCUGGCCGGCAUGGCCGACGGCCUGUUCCUGCUGAGACAGUGC |
| | CUGAGAAGCCUGGGCGGCUACGUGCUGAGCCUGGUGCACGACGUGAGAUUCCACCACU |
| | UCCCUAUCGAGAGACAGCUGAACGGCACCUACGCCAUCGCCGGCGGCAAGGCCCACUGC |
| | GGCCCUGCCGAGCUGUGCGAGUUCUACAGCCGUGAUCCUGACGGACUGCCUUGCAACC |
| | UGAGAAAGCCUUGCGGCGGCGGAGGUAGCGGCGGUGGUGGCAGUGGAGGCGGAGGAUC |
| | GGGAGGAGGCGGCUCCUGGUACCACAGCAGCCUGACCAGAGAGGAGGCCGAAAGAAAG |
| | CUGUACAGCGGCGCCCAGACCGACGGCAAGUUCCUGUUGCGCCCUAGAAAGGAGCAGG |
| | GCACUUACGCUCUGUCGUUAAUCUACGGCAAGACCGUGUACCACUACCUGAUCAGCCA |
| | GGACAAGGCCGGCAAGUACUGCAUCCCUGAGGGCACCAAGUUCGACACCCUGUGGCAG |
| | CUCGUGGAGUAUCUAAAGCUCAAGGCCGACGGCCUCAUCUACUGCCUGAAGGAGGCCU |
| | GUGGAGGUGGCGGAAGCGGAGGUGGUGGUUCCUUCUGGGAGGAGUUCGAGAGCCUGCA |
| | GAAGCAGGAGGUGAAGAACCUGCACCAGAGACUGGAGGGCCAGCGACCGGAGAACAAG |
| | GGCAAGAACAGAUACAAGAACAUUCUGCCGUUCGACCACUCACGCGUGAUCCUGCAGG |
| | GCCGAGAUAGCAACAUCCCUGGCAGCGACUACAUCAACGCCAAUUAUAUCAAGAACCA |
| | GCUGCUGGGCCCUGACGAGAACGCCAAGACCUACAUCGCCAGCCAGGGCUGCCUGGAG |
| | GCCACCGUGAACGACUUCUGGCAGAUGGCCUGGCAGGAGAAUUCUCGAGUCAUAGUGA |
| | UGACUACCCGGGAGGUGGAGAAGGGCAGAAACAAGUGCGUGCCUUACUGGCCUGAGGU |
| | GGGCAUGCAGAGAGCCUACGGCCCUUACAGCGUGACCAACUGCGGCGAGCACGACACC |
| | ACCGAGUACAAGCUGAGAACCCUGCAGGUGAGCCCUCUGGACAACGGCGAUCUGAUCA |
| | GAGAGAUCUGGCACUACCAAUACUUGUCUUGGCCUGAUCACGGCGUGCCUAGCGAGCC |
| | UGGCGGCGUAUUGUCUUUCCUGGACCAGAUCAAUCAGAGACAGGAGUCCCUCCCUCAC |
| | GCCGGCCCUAUCAUCGUGCACUGCAGCGCCGGCAUCGGCAGAACCGGCACCAUAAUUG |
| | UGAUCGACAUGCUGAUGGAGAACAUCAGCACCAAGGGCCUGGACUGCGACAUCGACAU |
| | CCAGAAGACCAUCCAGAUGGUGAGAGCCCAGAGAAGCGGCAUGGUGCAGACCGAGGCC |
| | CAGUACAAGUUCAUCUACGUGGCCAUAGCCCAGUUC |
| | [TCD4 nt. seq.] |
| 39 | UGGUUCUUCGGCAAGAUCCCUAGAGCCAAGGCCGAGGAGAUGCUGAGCAAGCAGAGAC |
| | ACGACGGCGCCUUCCUGAUCAGAGAGAGCGAGAGCGCCCCUGGCGACUUCAGCCUGAG |
| | CGUGAAGUUCGGCAACGACGUGCAGCACUUCAAGGUGCUGAGAGACGGCGCCGGCAAG |
| | UACUUCCUGUGGGUGGUGAAGUUCAACAGCCUGAACGAGCUGGUGGACUACCACAGAA |
| | GCACCAGCGUGAGCAGAAACCAGCAGAUCUUCCUGAGAGACAUUGAGGGCGGCGGAGG |
| | CAGCGGAGGAGGCGGAUCCGGAGGAGGAGGCUCCUUCUGGGAGGAGUUCGAGAGCCUG |
| | CAGAAGCAGGAGGUGAAGAACCUGCACCAGAGACUGGAGGGCCAAAGACCUGAGAACA |
| | AGGGCAAGAACAGAUACAAGAACAUCCUGCCUUUCGACCACAGCAGAGUGAUCCUGCA |
| | GGGCAGAGACAGCAACAUCCCUGGCAGCGACUACAUCAACGCCAACUACAUCAAGAAC |
| | CAGCUGCUGGGCCCUGACGAGAACGCCAAGACCUACAUCGCCAGCCAGGGCUGCCUGG |
| | AGGCCACCGUGAACGACUUCUGGCAGAUGGCCUGGCAGGAGAAUUCACGGGUCAUUGU |
| | GAUGACCACCAGAGAGGUGGAGAAGGGCAGAAACAAGUGCGUGCCUUACUGGCCUGAG |
| | GUGGGCAUGCAGAGAGCCUACGGCCCUUACAGCGUGACCAACUGCGGCGAGCACGACA |
| | CCACCGAGUACAAGCUGAGAACCCUGCAGGUGAGCCCUCUGGACAACGGCGACCUCAU |
| | ACGCGAGAUCUGGCACUACCAGUACCUGAGCUGGCCUGACCACGGCGUGCCUAGCGAG |
| | CCUGGCGGCGUGCUGAGCUUCCUGGACCAGAUCAACCAGAGACAGGAGAGCCUGCCUC |
| | ACGCCGGCCCUAUCAUCGUGCACUGCAGCGCCGGCAUCGGCAGAACCGGCACCAUCAUC |
| | GUGAUCGACAUGCUGAUGGAGAACAUCAGCACCAAGGGCCUGGACUGCGACAUCGACA |
| | UCCAGAAGACCAUCCAGAUGGUGAGAGCCCAGAGAAGCGGCAUGGUGCAGACCGAGGC |
| | CCAGUACAAGUUCAUCUACGUGGCCAUCGCCCAGUUC |
| | [TCD5 nt. seq.] |
| 40 | AUGUGGUACAGCGGCAGAAUCAGCAGACAGCUGGCCGAGGAGAUCCUGAUGAAGAGAA |
| | ACCACCUGGGCGCCUUCCUGAUCAGAGAGAGCGAGAGCAGCCCUGGCGAGUUCAGCGU |
| | GAGCGUGAACUACGGCGACCAGGUGCAGCACUUCAAGGUGCUGAGAGAGGCCAGCGGC |
| | AAGUACUUCCUGUGGGAGGAGAAGUUCAACAGCCUGAACGAGCUGGUGGACUUCUACA |
| | GAACCACCACCAUCGCCAAGAAGAGACAGAUCUUCCUGAGAGACGAGGAGCCUUUAGG |
| | AGGAGGCGGAUCUGGUGGUGGAGGCAGUGGCGGAGGCGGUUCGUUCUGGGAGGAGUUC |
| | GAGAGCCUGCAGAAGCAGGAGGUGAAGAACCUGCACCAGAGACUGGAGGGCCAAAGGC |
| | CUGAGAACAAGGGCAAGAACAGAUACAAGAACAUCCUGCCUUUCGACCACAGCAGAGU |
| | GAUCCUGCAGGGCAGAGACAGCAACAUCCCUGGCAGCGACUACAUCAACGCCAACUAC |
| | AUCAAGAACCAGCUGCUGGGCCCUGACGAGAACGCCAAGACCUACAUCGCCAGCCAGG |
| | GCUGCCUGGAGGCCACCGUGAACGACUUCUGGCAGAUGGCCUGGCAGGAGAACUCCCG |
| | GGUGAUUGUGAUGACCACCAGAGAGGUGGAGAAGGGCAGAAACAAGUGCGUGCCUUAC |
| | UGGCCUGAGGUGGGCAUGCAGAGAGCCUACGGCCCUUACAGCGUGACCAACUGCGGCG |
| | AGCACGACACCACCGAGUACAAGCUGAGAACCCUGCAGGUGAGCCCUCUGGACAACGG |
| | CGAUCUUAUUCGGGAGAUCUGGCACUACCAGUACCUGAGCUGGCCUGACCACGGCGUG |
| | CCUAGCGAGCCUGGCGGCGUGCUGAGCUUCCUGGACCAGAUCAACCAGAGACAGGAGA |
| | GCCUGCCUCACGCCGGCCCUAUCAUCGUGCACUGCAGCGCCGGCAUCGGCAGAACCGGC |
| | ACCAUCAUCGUGAUCGACAUGCUGAUGGAGAACAUCAGCACCAAGGGCCUGGACUGCG |
| | ACAUCGACAUCCAGAAGACCAUCCAGAUGGUGAGAGCCCAGAGAAGCGGCAUGGUGCA |
| | GACCGAGGCCCAGUACAAGUUCAUCUACGUGGCCAUCGCCCAGUUC |
| | [TCD6 nt. seq.] |
| 41 | AUGGGCUGCGGCUGCAGCAGCCACCCUGAGGACGACUGGAUGGAGAACAUCGACGUGU |
| | GCGAGAACUGCCACUACCCUAUCGUGCCUCUGGACGGCAAGGGCACCCUGCUGAUCAG |
| | AAACGGCAGCGAGGUGCGCGACCCUCUGGUGACCUACGAGGGCAGCAACCCUCCUGCC |
| | AGCCCUCUGCAGGACAACCUGGUGAUCGCCCUGCACAGCUACGAGCCUAGCCACGACGG |
| | CGACCUGGGCUUCGAGAAGGGCGAGCAGCUGAGAAUCCUGGAGCAGAGCGGCGAGUGG |
| | UGGAAGGCCCAGAGCCUGACCACCGGCCAGGAGGGCUUCAUCCCUUUCAACUUCGUGG |
| | CCAAGGCCAACAGCCUGGAGCCUGAGCCUUGGUUCUUCAAGAACCUGAGCAGAAAGGA |
| | CGCCGAGAGACAGCUGCUGGCCCCUGGCAACACCCACGGCAGCUUCCUGAUCAGAGAG |
| | AGCGAGAGCACCGCCGGAAGCUUCAGCCUGAGCGUGAGAGACUUCGACCAGAACCAGG |
| | GCGAGGUGGUGAAGCACUACAAGAUUAGGAACCUGGACAACGGCGGCUUCUACAUCAG |
| | CCCUAGAAUCACCUUCCCUGGCCUGCACGAGCUGGUGAGACACUACACCAACGCCAGCG |
| | ACGGCCUGUGCACCAGACUGAGCAGACCUUGCCAGACCCAGAAGCCUCAGAAGCCUUG |
| | GUGGGAGGACGAGUGGGAGGUGCCUAGAGAGACGGGCGGAGGAGGCUCCGGCGGUGGU |
| | GGCUCCGGAGGUGGCGGAUCCUUCUGGGAGGAGUUCGAGAGCCUGCAGAAGCAGGAGG |
| | UGAAGAACCUGCACCAGAGACUGGAGGGCCAACGCCCUGAGAACAAGGGCAAGAACAG |
| | AUACAAGAACAUCCUGCCUUUCGACCACAGCAGAGUGAUCCUGCAGGGCAGAGACAGC |
| | AACAUCCCUGGCAGCGACUACAUCAACGCCAACUAUAUCAAGAACCAACUGUUGGGCC |
| | CUGACGAGAACGCCAAGACCUACAUCGCCAGCCAGGGCUGCCUGGAGGCCACCGUGAA |
| | CGACUUCUGGCAGAUGGCCUGGCAGGAGAACUCUCGUGUGAUCGUGAUGACCACCAGA |
| | GAGGUGGAGAAGGGCAGAAACAAGUGCGUGCCUUACUGGCCUGAGGUGGGCAUGCAGA |
| | GAGCCUACGGCCCUUACAGCGUGACCAACUGCGGCGAGCACGACACCACCGAGUACAA |
| | GCUGAGAACCCUGCAGGUGAGCCCUCUGGAUAACGGAGAUCUGAUUAGGGAGAUCUGG |
| | CACUACCAGUACCUGAGCUGGCCGGACCACGGCGUGCCUAGCGAGCCUGGCGGCGUGC |
| | UGAGCUUCCUGGACCAGAUCAACCAGAGACAGGAGAGUCUGCCUCACGCCGGCCCUAU |
| | CAUCGUGCACUGCAGCGCCGGCAUCGGCAGAACCGGCACCAUCAUCGUGAUCGACAUG |
| | UUGAUGGAGAAUAUCAGCACCAAGGGCCUGGACUGCGACAUCGACAUCCAGAAGACCA |
| | UCCAGAUGGUGAGAGCCCAGAGAAGCGGCAUGGUGCAGACCGAGGCCCAGUACAAGUU |
| | CAUCUACGUGGCCAUCGCCCAGUUC |
| | [TCD7 nt. seq.] |
| 42 | AUGGAGGAGGCCAUCCUGGUGCCUUGCGUGCUGGGCCUGCUGUUAUUACCUAUCCUGG |
| | CCAUGCUGAUGGCCCUGUGCGUGCACUGCCACAGACUGCCUGGCAGCUACGACAGCACC |
| | AGCAGCGACAGCCUGUACCCUAGAGGCAUCCAGUUCAAGCGGCCUCACACCGUGGCCCC |
| | UUGGCCUCCUGCCUACCCUCCUGUGACCAGUUAUCCUCCUCUGAGCCAGCCUGACCUAC |
| | UACCUAUCCCUAGAAGCCCUCAGCCUCUGGGCGGCAGCCACAGAACCCCUAGCAGCAGA |
| | AGAGACAGCGACGGCGCCAACAGCGUGGCCAGCUACGAGAACGAGGGCGCCAGCGGCA |
| | UCAGAGGAGCACAGGCCGGCUGGGGCGUGUGGGGCCCUAGCUGGACCAGACUGACCCC |
| | UGUGAGCCUGCCUCCAGAACCUGCUUGCGAGGACGCCGACGAGGACGAGGACGACUAC |
| | CACAACCCUGGCGUGACCUACGCCCAGUUGCUGCCUGACAGCACCCCUGCCACCAGCAC |
| | CGCCGCCCCUAGCGCCCCUGCCCUGUCAACCCCAGGAAUUCGUGAUUCGGCCUUCAGCA |
| | UGGAGAGCAUCGACGACGUUACCUACGCACAACUGCCUGAGAGCGGCGAGAGCGCCGA |
| | GGCCAGCCUGGACGGCAGCAGAGAGGUCACCUACGCACAGCUGAGCCAGGAGCUGCAC |
| | CCUGGCGCCGCCAAGACCGAGCCUGCCGCUCUUAGCAGCCAGGAGGCCGAGGAGGUGG |
| | AGGAGGAGGGCGCCCCUGACUACGAGAACCUGCAGGAGCUGAACAUCCCUAACCCUUU |
| | GUUGGGUCUGGACUGAUAAUAGGCUGGAGCCUCGGUGGCCUAGCUUCUUGCCCCUUGG |
| | GCCUCCCCCCAGCCCCUCCUCCCCUUCCUGCACCCGUACCCCCGUGG |
| | [TCD8 nt. seq.] |
| 43 | AUGGAGGAGGCCAUCCUGGUGCCUUGCGUGCUGGGCCUGCUGCUGCUGCCUAUCCUGG |
| | CCAUGCUGAUGGCCCUGUGCGUGCACUGCCACAGACUGCCUGGCAGCUACGACAGCACC |
| | AGCAGCGACAGCCUGUACCCUAGAGGCAUCCAGUUCAAGCGGCCUCACACCGUGGCCCC |
| | UUGGCCUCCUGCCUACCCUCCUGUGACCAGCUACCCUCCUCUGAGCCAGCCUGACCUGC |
| | UCCCUAUCCCUAGAAGCCCUCAGCCUCUGGGCGGCAGCCACAGAACCCCUAGCAGCAGA |
| | AGAGACAGCGACGGCGCCAACAGCGUGGCCAGCUACGAGAACGAGGGCGCCAGCGGCA |
| | UCAGAGGCGCUCAGGCAGGCUGGGGCGUGUGGGGCCCUAGCUGGACCAGACUGACCCC |
| | UGUGAGCCUGCCUCCAGAGCCUGCCUGCGAGGACGCCGACGAGGACGAGGACGACUAC |
| | CACAACCCUGGCAUCACCUACGCCGCCGUGCUGCCUGACAGCACCCCUGCCACCAGCAC |
| | CGCCGCCCCUAGCGCCCCUGCCCUGAGCACGCCUGGAAUCAGAGACAGCGCCUUCAGCA |
| | UGGAGAGCAUCGACGAUAUUACAUACGCUGCCGUGCCUGAGAGCGGCGAGAGCGCCGA |
| | GGCCAGCCUGGACGGCAGCAGAGAGAUUACAUACGCAGCCGUGAGCCAGGAGCUGCAC |
| | CCUGGCGCCGCCAAGACCGAGCCUGCCGCCCUGAGCAGCCAGGAGGCCGAGGAGGUGG |
| | AGGAGGAGGGCGCCCCUGACUACGAGAACCUGCAGGAGCUGAAC |
| | [TCD9 nt. seq.] |
| 44 | AUGGAGGAGGCCAUCCUGGUGCCUUGCGUGCUGGGCCUGCUAUUACUUCCUAUCCUGG |
| | CCAUGCUGAUGGCCCUGUGCGUGCACUGCCACAGACUGCCUGGCAGCUACGACAGCACC |
| | AGCAGCGACAGCCUGUACCCUAGAGGCAUCCAGUUCAAGAGGCCUCACACCGUGGCCCC |
| | UUGGCCUCCUGCCUACCCUCCUGUGACCUCUUACCCGCCACUGAGCCAGCCUGACCUAU |
| | UGCCAAUACCUAGAAGCCCUCAGCCUCUGGGCGGCAGCCACAGAACCCCUAGCAGCAGA |
| | AGAGACAGCGACGGCGCCAACAGCGUGGCGAGCUACGAGAACGAGGGCGCCAGCGGCA |
| | UCAGAGGUGCCCAGGCAGGCUGGGGCGUGUGGGGCCCUAGCUGGACCAGACUGACCCC |
| | UGUGAGCCUGCCUCCAGAGCCUGCUUGCGAGGACGCCGACGAGGACGAAGACGACUAC |
| | CACAACCCUGGCGCCCUGGUGGUGCUGCCUGACUCCACACCUGCCACCAGCACCGCCGC |
| | CCCUAGCGCCCCUGCCCUGAGCACUCCUGGUAUUCGUGAUAGCGCCUUCAGCAUGGAG |
| | AGCAUCGACGACGCCGUGAACGUGCCUGAGAGCGGCGAGAGCGCCGAGGCUUCCCUCG |
| | ACGGCAGCAGAGAGGCUGUGAACGUGAGCCAGGAGCUGCAUCCAGGUGCAGCCAAGAC |
| | CGAACCUGCCGCUCUGAGCAGUCAGGAGGCCGAGGAGGUGGAAGAGGAGGGUGCACCA |
| | GACUACGAGAAUCUGCAGGAGUUAAACCACAGACAGAACCAGAUCAAGCAGGGCCCUC |
| | CUAGGUCUAAGGACGAGGAGCAGAAGCCUCAGCAGCGGCCGGAUUUGGCCGUGGACGU |
| | GCUGGAGAGAACCGCCGACAAGGCCACCGUUAACGGAUUGCCAGAGAAGGACAGAGAG |
| | ACUGACACCAGCGCACUGGCCGCGGGUUCCUCUCAGGAGGUGACCUACGCCCAGCUCGA |
| | UCACUGGGCCCUGACCCAGAGGACUGCCAGAGCCGUGAGUCCACAGAGCACCAAGCCU |
| | AUGGCCGAAAGCAUUACUUACGCCGCCGUGGCCAGACAC |
| | [TCD10 nt. seq.] |
| 45 | AUGGAGGAGGCCAUCCUGGUGCCUUGCGUGCUGGGCCUGCUCCUCUUGCCUAUCCUGG |
| | CCAUGCUGAUGGCCCUGUGCGUGCACUGCCACAGACUGCCUGGCAGCUACGACAGCACC |
| | AGCAGCGACAGCCUGUACCCUAGAGGCAUCCAGUUCAAGCGCCCUCACACCGUGGCCCC |
| | UUGGCCUCCUGCCUACCCUCCUGUGACCAGUUACCCGCCACUGAGCCAGCCUGACCUCU |
| | UACCUAUCCCUAGAAGCCCUCAGCCUCUGGGCGGCAGCCACAGAACCCCUAGCAGCAGA |
| | AGAGACAGCGACGGCGCCAACAGCGUGGCUUCGUACGAGAACGAGGGCGCCAGCGGCA |
| | UCAGAGGAGCUCAAGCUGGUUGGGGCGUGUGGGGCCCUAGCUGGACCAGACUGACCCC |
| | UGUGAGCCUGCCUCCGGAGCCAGCGUGCGAGGACGCCGACGAGGACGAGGACGACUAC |
| | CACAACCCUGGCGCCCUGGUGGUGCUGCCUGACUCUACUCCUGCCACCAGCACCGCCGC |
| | CCCUAGCGCCCCUGCCCUGAGCACGCCUGGCAUCCGCGACUCGGCCUUCAGCAUGGAGA |
| | GCAUCGACGACGCCGUGAACGUGCCUGAGAGCGGCGAGAGCGCCGAGGCCUCUUUAGA |
| | CGGCAGCAGAGAGGCCGUAAACGUCAGCCAGGAGCUGCACCCAGGUGCAGCCAAGACC |
| | GAACCGGCUGCCCUCUCCAGUCAAGAAGCCGAGGAGGUAGAAGAGGAGGGUGCGCCAG |
| | ACUACGAGAAUCUGCAGGAACUCAACGGAGGAGGUGGCAGCGGUGGCGGCGGAAGCUU |
| | CUGGGAGGAGUUCGAGAGCCUGCAGAAGCAGGAGGUGAAGAACCUUCACCAGAGACUG |
| | GAGGGCCAACGCCCUGAGAACAAGGGCAAGAACAGAUACAAGAACAUCCUGCCUUUCG |
| | ACCACAGCAGAGUGAUCCUGCAGGGCAGAGAUUCCAACAUCCCUGGCUCAGACUACAU |
| | CAACGCCAAUUACAUCAAGAACCAGUUGCUCGGACCUGACGAGAACGCUAAGACUUAC |
| | AUCGCCAGCCAGGGCUGCCUGGAGGCCACUGUGAACGACUUCUGGCAGAUGGCUUGGC |
| | AAGAGAAUUCUCGGGUUAUUGUGAUGACCACACGUGAAGUUGAGAAGGGCAGAAACAA |
| | GUGCGUGCCUUACUGGCCUGAGGUGGGCAUGCAGAGAGCCUACGGCCCUUACAGCGUG |
| | ACCAACUGCGGCGAGCACGACACCACCGAGUACAAGCUGAGAACCCUGCAGGUGAGCC |
| | CUCUGGACAACGGCGACCUGAUCAGAGAGAUCUGGCACUACCAGUACCUGAGUUGGCC |
| | AGACCACGGCGUGCCUAGCGAGCCUGGCGGCGUGCUGAGCUUCCUGGACCAAAUCAAC |
| | CAGCGCCAAGAGUCUCUCCCACACGCCGGCCCUAUCAUCGUGCAUUGCAGCGCCGGCAU |
| | CGGCAGAACCGGCACCAUUAUCGUGAUCGAUAUGUUGAUGGAGAACAUCAGCACCAAG |
| | GGCCUGGACUGCGACAUCGACAUCCAGAAGACCAUCCAGAUGGUGAGAGCCCAGAGAA |
| | GCGGCAUGGUGCAGACCGAGGCCCAGUACAAGUUCAUCUACGUGGCCAUCGCCCAGUU |
| | C |
| | [TCD11 nt. seq.] |
| 46 | AUGGAGGAGGCCAUCCUGGUGCCUUGCGUGCUGGGCCUGCUGCUGCUGCCUAUCCUGG |
| | CCAUGCUGAUGGCCCUGUGCGUGCACUGCCACAGACUGCCUGGCAGCUACGACAGUGG |
| | CUUCGGAGGCGGAGGCUCUGGUGGCGGUGGCUCUGGAGGUGGAGGCAGUUUCUGGGAG |
| | GAGUUCGAGAGCCUGCAGAAGCAGGAGGUGAAGAACCUGCACCAGAGACUGGAGGGCC |
| | AACGGCCUGAGAACAAGGGCAAGAACAGAUACAAGAACAUCCUGCCUUUCGACCACAG |
| | CAGAGUGAUCCUGCAGGGCAGAGACAGCAACAUCCCUGGCAGCGACUACAUCAACGCC |
| | AACUACAUCAAGAACCAGCUGCUGGGCCCUGACGAGAACGCCAAGACCUACAUCGCCA |
| | GCCAGGGCUGCCUGGAGGCCACCGUGAACGACUUCUGGCAGAUGGCCUGGCAGGAGAA |
| | CUCAAGGGUGAUAGUGAUGACCACCAGAGAGGUGGAGAAGGGCAGAAACAAGUGCGUG |
| | CCUUACUGGCCUGAGGUGGGCAUGCAGAGAGCCUACGGCCCUUACAGCGUGACCAACU |
| | GCGGCGAGCACGACACCACCGAGUACAAGCUGAGAACCCUGCAGGUGAGCCCUCUGGA |
| | CAACGGCGACCUGAUCAGAGAGAUCUGGCACUACCAGUACCUGAGCUGGCCUGACCAC |
| | GGCGUGCCUAGCGAGCCUGGCGGCGUGCUGAGCUUCCUGGACCAGAUCAACCAGAGAC |
| | AGGAGAGCCUGCCUCACGCCGGCCCUAUCAUCGUGCACUGCAGCGCCGGCAUCGGCAGA |
| | ACCGGCACCAUCAUCGUGAUCGACAUGCUGAUGGAGAACAUCAGCACCAAGGGCCUGG |
| | ACUGCGACAUCGACAUCCAGAAGACCAUCCAGAUGGUGAGAGCCCAGAGAAGCGGCAU |
| | GGUGCAGACCGAGGCCCAGUACAAGUUCAUCUACGUGGCCAUCGCCCAGUUC |
| | [TCD12 nt. seq.] |
| 47 | AUGGAGGAGGCCAUCCUGGUGCCUUGCGUGCUGGGCCUGCUGCUGCUGCCUAUCCUGG |
| | CCAUGCUGAUGGCCCUGUGCGUGCACUGCCACAGACUGCCUGGCAGCUACGACAGCCAC |
| | AGACAGAACCAGAUCAAGCAGGGCCCUCCUAGAAGCAAGGACGAGGAGCAGAAGCCUC |
| | AGCAGAGACCUGACCUGGCCGUGGACGUGCUGGAGAGAACCGCCGACAAGGCCACCGU |
| | GAACGGCCUGCCUGAGAAGGACAGAGAGACCGACACCAGCGCCCUGGCCGCCGGCAGC |
| | AGCCAGGAGGUGACCUACGCCCAGCUGGACCACUGGGCCCUGACCCAGAGAACCGCCAG |
| | AGCCGUGAGCCCUCAGAGCACCAAGCCUAUGGCCGAGAGCAUCACCUACGCCGCCGUGG |
| | CCAGACAC |
| | [TCD13 nt. seq.] |
| 48 | AUGGAGGAGGCCAUCCUGGUGCCUUGCGUGCUGGGCCUGCUCCUGCUCCCUAUCCUGG |
| | CCAUGCUGAUGGCCCUGUGCGUGCACUGCCACAGACUGCCUAUGAGCGCCAUCCAGGCC |
| | GCCUGGCCUAGCGGCACCGAGUGCAUCGCCAAGUACAACUUCCACGGCACCGCCGAGCA |
| | GGACCUGCCUUUCUGCAAGGGCGACGUGCUGACCAUCGUGGCCGUGACCAAGGACCCU |
| | AACGCCUACAAGGCCAAGAACAAGGUGGGCAGAGAGGGCAUCAUCCCUGCCAACUACG |
| | UGCAGAAGAGAGAGGGCGUGAAGGCCGGCACCAAGCUGAGCCUGAUGCCUUGGUUCCA |
| | CGGCAAGAUCACCAGAGAGCAGGCCGAGAGACUGCUGUACCCUCCUGAGACGGGCCUG |
| | UUCCUGGUGAAGGAGAGCACCAACUACCCUGGCGACUACACUCUUUGCGUGAGCUGCG |
| | ACGGCAAGGUGGAGCACUACAGAAUCAUGUACCACGCCAGUAAGCUGAGCAUCGACGA |
| | GGAGGUGUACUUCGAGAACCUGAUGCAGCUGGUGGCCCACUACACCAGCGACGCCGAC |
| | GGCCUGUGCACCAGACUGAUCAAGCCUAAGGUGAUGGAGGGCACCGUGGCCGCCCAGG |
| | ACGAGUUCUACAGAAGCGGCUGGGCCCUGAACAUGAAGGAGCUGAAGCUGCUGCAGAC |
| | CAUCGGAAAGGGCGAGUUCGGCGACGUGAUGCUGGGCGAUUACAGAGGCAAUAAGGUC |
| | GCUGUUAAGUGCAUCAAGAACGACGCCACCGCCCAGGCCUUCCUGGCCGAGGCCAGCG |
| | UGAUGACCCAGCUGAGACACAGCAACCUGGUGCAGCUGCUGGGCGUGAUCGUGGAGGA |
| | GAAGGGCGGCCUGUACAUCGUGACCGAGUACAUGGCCAAGGGCAGCCUGGUGGACUAC |
| | CUGAGAAGCAGAGGCAGAUCUGUUCUGGGCGGCGACUGCCUGCUGAAGUUCAGCCUGG |
| | ACGUGUGCGAGGCCAUGGAGUACCUGGAGGGCAACAACUUCGUGCACCGGGAUCUGGC |
| | CGCCAGAAACGUGCUGGUGAGCGAGGACAACGUGGCCAAGGUGAGCGACUUCGGCCUA |
| | ACGAAGGAGGCCAGCAGCACCCAGGACACCGGCAAGCUGCCUGUGAAGUGGACCGCCC |
| | CUGAGGCCCUGAGAGAGAAGAAGUUCAGCACCAAGAGCGACGUGUGGAGCUUCGGCAU |
| | CCUGCUGUGGGAGAUCUACUCCUUCGGCAGAGUGCCUUACCCUAGAAUCCCUCUGAAG |
| | GACGUGGUGCCUAGAGUUGAGAAGGGCUACAAGAUGGACGCCCCUGACGGCUGCCCUC |
| | CUGCCGUGUACGAGGUGAUGAAGAACUGCUGGCACCUGGACGCCGCCAUGCGACCUAG |
| | CUUCCUGCAGCUGAGAGAGCAGCUGGAGCACAUCAAGACCCACGAGCUGCACCUG |
| | [TCD14 nt. seq.] |
| 49 | AUGGGCCCUGCCGGCAGCCUGCUGGGCAGCGGCCAGAUGCAGAUCACCCUGUGGGGCA |
| | GCCUGGCCGCCGUGGCCAUCUUCUUCGUGAUCACCUUCCUGAUCUUCCUGUGCAGCAGC |
| | UGCGACAGAGAGAAGAAGCCUAGAAUGAGCGCCAUCCAGGCCGCCUGGCCUAGCGGCA |
| | CCGAGUGCAUCGCCAAGUACAACUUCCACGGCACCGCCGAGCAGGACCUGCCUUUCUGC |
| | AAGGGCGACGUGCUGACCAUCGUGGCCGUGACCAAGGACCCUAACGCCUACAAGGCCA |
| | AGAACAAGGUGGGCAGAGAGGGCAUCAUCCCUGCCAACUACGUGCAGAAGAGAGAGGG |
| | CGUGAAGGCCGGCACCAAGCUGAGCCUGAUGCCUUGGUUCCACGGAAAGAUCACCAGA |
| | GAGCAGGCCGAGAGACUGCUGUACCCUCCUGAAACUGGCCUGUUCCUGGUGAAGGAGA |
| | GCACCAACUACCCUGGCGACUACACCCUGUGCGUGAGCUGCGACGGCAAGGUGGAGCA |
| | CUACAGAAUCAUGUACCACGCCAGCAAGUUAAGCAUCGACGAGGAGGUGUACUUCGAG |
| | AACCUGAUGCAGCUGGUGGCCCACUACACCAGCGACGCCGACGGCCUGUGCACCAGACU |
| | GAUCAAGCCUAAGGUGAUGGAGGGCACCGUGGCCGCCCAGGACGAGUUCUACAGAAGC |
| | GGCUGGGCCCUGAACAUGAAGGAGCUGAAGCUGCUGCAGACCAUCGGCAAGGGUGAGU |
| | UCGGCGACGUGAUGCUGGGAGACUACAGAGGCAAUAAGGUAGCAGUAAAGUGCAUCAA |
| | GAACGACGCCACCGCCCAGGCCUUCCUGGCCGAGGCCAGCGUGAUGACCCAGCUGAGAC |
| | ACAGCAACCUGG |
| | [TCD15 nt. seq.] |
| 50 | AUGGGCUGCGGCUGCAGCAGCCACCCUGAGGACGACUGGAUGGAGAACAUCGACGUGU |
| | GCGAGAACUGCCACUACCCUAUCGUGCCUCUGGACGGCAAGGGCACCCUGCUGAUCAG |
| | AAACGGCAGCGAGGUGCGAGAUCCUCUGGUGACCAUGAGCGCCAUCCAGGCCGCCUGG |
| | CCUAGCGGCACCGAGUGCAUCGCCAAGUACAACUUCCACGGCACCGCCGAGCAGGACCU |
| | GCCUUUCUGCAAGGGCGACGUGCUGACCAUCGUGGCCGUGACCAAGGACCCUAACGCC |
| | UACAAGGCCAAGAACAAGGUGGGCAGAGAGGGCAUCAUCCCUGCCAACUACGUGCAGA |
| | AGAGAGAGGGCGUGAAGGCCGGCACCAAGCUGAGCCUGAUGCCUUGGUUCCACGGCAA |
| | GAUCACCAGAGAGCAGGCCGAGAGACUGCUGUACCCUCCUGAGACUGGCCUGUUCCUG |
| | GUGAAGGAGAGCACCAACUACCCUGGCGACUACACCCUGUGCGUGAGCUGCGACGGAA |
| | AGGUGGAGCACUACAGAAUCAUGUACCACGCCUCUAAGCUCAGCAUCGACGAGGAGGU |
| | GUACUUCGAGAACCUGAUGCAGCUGGUGGCCCACUACACCAGCGACGCCGACGGCCUG |
| | UGCACCAGACUGAUCAAGCCUAAGGUGAUGGAGGGCACCGUGGCCGCCCAGGACGAGU |
| | UCUACAGAAGCGGCUGGGCCCUGAACAUGAAGGAGCUGAAGCUGCUGCAGACCAUCGG |
| | AAAGGGCGAGUUCGGCGACGUGAUGCUGGGAGACUAUAGAGGCAAUAAGGUAGCCGUC |
| | AAGUGCAUCAAGAACGACGCCACCGCCCAGGCCUUCCUGGCCGAGGCCAGCGUGAUGA |
| | CCCAGCUGAGACACAGCAACCUGGUGCAGCUGCUGGGCGUGAUCGUGGAGGAGAAGGG |
| | CGGCCUGUACAUCGUGACCGAGUACAUGGCCAAGGGCAGCCUGGUGGACUACCUGAGA |
| | AGCAGAGGCAGAAGCGUGCUGGGCGGCGACUGCCUGCUGAAGUUCAGCCUGGACGUGU |
| | GCGAGGCCAUGGAGUACCUGGAGGGCAACAACUUCGUGCACCGGGAUCUGGCCGCCAG |
| | AAACGUGCUGGUGAGCGAGGACAACGUGGCCAAGGUGAGCGACUUCGGCCUAACAAAG |
| | GAGGCCAGCAGCACCCAGGACACCGGCAAGCUGCCUGUGAAGUGGACCGCCCCUGAGG |
| | CCCUGAGAGAGAAGAAGUUCAGCACCAAGAGCGACGUGUGGAGCUUCGGCAUCCUGCU |
| | GUGGGAGAUCUACUCAUUCGGCAGAGUGCCUUACCCUAGAAUCCCUCUGAAGGACGUG |
| | GUGCCUAGAGUCGAGAAGGGCUACAAGAUGGACGCCCCUGACGGCUGCCCUCCUGCCG |
| | UGUACGAGGUGAUGAAGAACUGCUGGCACCUGGACGCCGCCAUGCGACCUAGCUUCCU |
| | GCAGCUGAGAGAGCAGCUGGAGCACAUCAAGACCCACGAGCUGCACCUG |
| | [TCD16 nt. seq.] |
| 51 | AUGGGCUGCGUGCAGUGCAAGGACAAGGAGGCCACCAAGCUGACCGAGGAGAGAGACG |
| | GCAGCCUGAACCAGAGCAGCGGCUACAGAUACGGCACCGACCCUACCCCUCAGCACUAC |
| | CCUAGCUUCGGCGUGACCAGCAUCCCUAACUACAUGAGCGCCAUCCAGGCCGCCUGGCC |
| | UAGCGGCACCGAGUGCAUCGCCAAGUACAACUUCCACGGCACCGCCGAGCAGGACCUGC |
| | CUUUCUGCAAGGGCGACGUGCUGACCAUCGUGGCCGUGACCAAGGACCCUAACGCCUA |
| | CAAGGCCAAGAACAAGGUGGGCAGAGAGGGCAUCAUCCCUGCCAACUACGUGCAGAAG |
| | AGAGAGGGCGUGAAGGCCGGAACAAAGUUAAGCCUGAUGCCUUGGUUCCACGGCAAGA |
| | UCACCAGAGAGCAGGCCGAGAGACUGCUGUACCCUCCUGAGACUGGCCUGUUCCUGGU |
| | GAAGGAGAGCACCAACUACCCUGGCGACUACACCCUGUGCGUGAGCUGCGACGGCAAG |
| | GUGGAGCACUACAGAAUCAUGUACCACGCCUCUAAGCUCAGCAUCGACGAGGAGGUGU |
| | ACUUCGAGAACCUGAUGCAGCUGGUGGCCCACUACACCAGCGACGCCGACGGCCUGUG |
| | CACCAGACUGAUCAAGCCUAAGGUGAUGGAGGGCACCGUGGCCGCCCAGGACGAGUUC |
| | UACAGAAGCGGCUGGGCCCUGAACAUGAAGGAGCUGAAGCUGCUGCAGACCAUCGGUA |
| | AGGGUGAGUUCGGCGACGUGAUGCUGGGCGACUAUAGAGGCAAUAAGGUGGCUGUGAA |
| | GUGCAUCAAGAACGACGCCACCGCCCAGGCCUUCCUGGCCGAGGCCAGCGUGAUGACCC |
| | AGCUGAGACACAGCAACCUGGUGCAGCUGCUGGGCGUGAUCGUGGAGGAGAAGGGCGG |
| | CCUGUACAUCGUGACCGAGUACAUGGCCAAGGGCAGCCUGGUGGACUACCUGAGAAGC |
| | AGAGGCAGAAGCGUGCUGGGCGGCGACUGCCUGCUGAAGUUCAGCCUGGACGUGUGCG |
| | AGGCCAUGGAGUACCUGGAGGGCAACAACUUCGUGCACCGAGAUCUGGCCGCCAGAAA |
| | CGUGCUGGUGAGCGAGGACAACGUGGCCAAGGUGAGCGACUUCGGCCUUACUAAGGAA |
| | GCAAGCAGCACCCAGGACACCGGCAAGCUGCCAGUAAAGUGGACCGCCCCUGAGGCCCU |
| | GAGAGAGAAGAAGUUCAGCACCAAGAGCGACGUGUGGAGCUUCGGCAUCCUGCUGUGG |
| | GAGAUCUACUCUUUCGGAAGAGUGCCUUACCCUAGAAUCCCUCUGAAGGACGUGGUGC |
| | CUAGAGUAGAGAAGGGCUACAAGAUGGACGCCCCUGACGGCUGCCCUCCUGCCGUGUA |
| | CGAGGUGAUGAAGAACUGCUGGCACCUGGACGCCGCCAUGCGGCCUAGCUUCCUGCAG |
| | CUGAGAGAGCAGCUGGAGCACAUCAAGACCCACGAGCUGCACCUG |
| | [TCD17 nt. seq.] |
| 52 | AUGGGCAGCAACAAGAGCAAGCCUAAGGACAUGAGCGCCAUCCAGGCCGCCUGGCCUA |
| | GCGGCACCGAGUGCAUCGCCAAGUACAACUUCCACGGCACCGCCGAGCAGGACCUGCCU |
| | UUCUGCAAGGGCGACGUGCUGACCAUCGUGGCCGUGACCAAGGACCCUAACGCCUACA |
| | AGGCCAAGAACAAGGUGGGCAGAGAGGGCAUCAUCCCUGCCAACUACGUGCAGAAGAG |
| | AGAGGGCGUGAAGGCCGGCACCAAGCUGAGCCUGAUGCCUUGGUUCCACGGAAAGAUC |
| | ACCAGAGAGCAGGCCGAGAGACUGCUGUACCCUCCUGAGACAGGCCUGUUCCUGGUGA |
| | AGGAGAGCACCAACUACCCUGGCGACUACACCCUGUGCGUGAGCUGCGACGGCAAGGU |
| | GGAGCACUACAGAAUCAUGUACCACGCCAGUAAGCUCAGCAUCGACGAGGAGGUGUAC |
| | UUCGAGAACCUGAUGCAGCUGGUGGCCCACUACACCAGCGACGCCGACGGCCUGUGCA |
| | CCAGACUGAUCAAGCCGAAGGUGAUGGAGGGCACCGUGGCCGCCCAGGACGAGUUCUA |
| | CAGAAGCGGCUGGGCCCUGAACAUGAAGGAGCUGAAGCUGCUGCAGACCAUCGGAAAG |
| | GGAGAGUUCGGCGACGUGAUGCUGGGUGACUACAGAGGCAACAAGGUAGCUGUCAAGU |
| | GCAUCAAGAACGACGCCACCGCCCAGGCCUUCCUGGCCGAGGCCAGCGUGAUGACCCAG |
| | CUGAGACACAGCAACCUGGUGCAGCUGCUGGGCGUGAUCGUGGAGGAGAAGGGCGGCC |
| | UGUACAUCGUGACCGAGUACAUGGCCAAGGGCAGCCUGGUGGACUACCUGAGAAGCAG |
| | AGGCAGAAGCGUGCUGGGCGGCGACUGCCUGCUGAAGUUCAGCCUGGACGUGUGCGAG |
| | GCCAUGGAGUACCUGGAGGGCAACAACUUCGUGCACCGAGAUCUGGCCGCCAGAAACG |
| | UGCUGGUGAGCGAGGACAACGUGGCCAAGGUGAGCGACUUCGGCUUGACUAAGGAGGC |
| | CAGCAGCACCCAGGACACCGGCAAGCUGCCUGUGAAGUGGACCGCCCCUGAGGCCCUGA |
| | GAGAGAAGAAGUUCAGCACCAAGAGCGACGUGUGGAGCUUCGGCAUCCUGCUGUGGGA |
| | GAUCUACUCUUUCGGUAGAGUGCCUUACCCUAGAAUCCCUCUGAAGGACGUGGUGCCU |
| | AGAGUUGAGAAGGGCUACAAGAUGGACGCCCCUGACGGCUGCCCUCCUGCCGUGUACG |
| | AGGUGAUGAAGAACUGCUGGCACCUGGACGCCGCCAUGAGGCCUAGCUUCCUGCAGCU |
| | GAGAGAGCAGCUGGAGCACAUCAAGACCCACGAGCUGCACCUG |
| | [TCD18 nt. seq.] |
| 53 | AUGGAGGAGGCCAUCCUGGUGCCUUGCGUGCUGGGCCUGCUGCUGCUGCCUAUCCUGG |
| | CCAUGCUGAUGGCCCUGUGCGUGCACUGCCACAGACUGCCUCUGAAGCUGCUGCAGAC |
| | CAUCGGCAAGGGCGAGUUCGGCGACGUGAUGCUGGGCGACUACAGAGGCAACAAGGUG |
| | GCCGUGAAGUGCAUCAAGAACGACGCCACCGCCCAGGCCUUCCUGGCCGAGGCCAGCGU |
| | GAUGACCCAGCUGAGACACAGCAACCUGGUGCAGCUGCUGGGCGUGAUCGUGGAGGAG |
| | AAGGGCGGCCUGUACAUCGUGACCGAGUACAUGGCCAAGGGCAGCCUGGUGGACUACC |
| | UGAGAAGCAGAGGCAGAAGUGUGCUAGGCGGCGACUGCCUGCUGAAGUUCAGCCUGGA |
| | CGUGUGCGAGGCCAUGGAGUACCUGGAGGGCAACAACUUCGUGCACCGCGACCUGGCC |
| | GCCAGAAACGUGCUGGUGAGCGAGGACAACGUGGCCAAGGUGAGCGACUUCGGCCUGA |
| | CCAAGGAGGCCAGCAGCACCCAGGACACCGGCAAGCUGCCUGUGAAGUGGACCGCCCCU |
| | GAGGCCCUGAGAGAGAAGAAGUUCAGCACCAAGAGCGACGUGUGGAGCUUCGGCAUCC |
| | UGCUGUGGGAGAUCUACAGCUUCGGCAGAGUGCCUUACCCUAGAAUCCCUCUGAAGGA |
| | CGUGGUGCCUAGAGUGGAGAAGGGCUACAAGAUGGACGCCCCUGACGGCUGCCCUCCU |
| | GCCGUGUACGAGGUGAUGAAGAACUGCUGGCACCUGGACGCCGCCAUGCGACCUAGCU |
| | UCCUGCAGCUGAGAGAGCAGCUGGAGCACAUCAAGACCCACGAGCUGCAC |
| | [TCD19 nt. seq.] |
| 54 | AUGGGCCCUGCCGGCAGCCUGCUGGGCAGCGGCCAGAUGCAGAUCACCCUGUGGGGCA |
| | GCCUGGCCGCCGUGGCCAUCUUCUUCGUGAUCACCUUCCUGAUCUUCCUGUGCAGCAGC |
| | UGCGACAGAGAGAAGAAGCCUAGACUGAAGCUGCUGCAGACCAUCGGCAAGGGCGAGU |
| | UCGGCGACGUGAUGCUGGGCGACUACAGAGGCAACAAGGUGGCCGUGAAGUGCAUCAA |
| | GAACGACGCCACCGCCCAGGCCUUCCUGGCCGAGGCCAGCGUGAUGACCCAGCUGAGAC |
| | ACAGCAACCUGGUGCAGCUGCUGGGCGUGAUCGUGGAGGAGAAGGGCGGCCUGUACAU |
| | CGUGACCGAGUACAUGGCCAAGGGCAGCCUGGUGGACUACCUGAGAAGCAGAGGCAGA |
| | AGCGUGCUGGGCGGCGACUGCCUGCUGAAGUUCAGCCUGGACGUGUGCGAGGCCAUGG |
| | AGUACCUGGAGGGCAACAACUUCGUGCACAGAGACCUGGCCGCCAGAAACGUGCUGGU |
| | GAGCGAGGACAACGUGGCCAAGGUGAGCGACUUCGGCCUGACCAAGGAGGCCAGCAGC |
| | ACCCAGGACACCGGCAAGCUGCCUGUGAAGUGGACCGCCCCUGAGGCCCUGAGAGAGA |
| | AGAAGUUCAGCACCAAGAGCGACGUGUGGAGCUUCGGCAUCCUGCUGUGGGAGAUCUA |
| | CAGCUUCGGCAGAGUGCCUUACCCUAGAAUCCCUCUGAAGGACGUGGUGCCUAGAGUG |
| | GAGAAGGGCUACAAGAUGGACGCCCCUGACGGCUGCCCUCCUGCCGUGUACGAGGUGA |
| | UGAAGAACUGCUGGCACCUGGACGCCGCCAUGAGACCUAGCUUCCUGCAGCUGAGAGA |
| | GCAGCUGGAGCACAUCAAGACCCACGAGCUGCAC |
| | [TCD20 nt. seq.] |
| 55 | AUGGGCUGCGGCUGCAGCAGCCACCCUGAGGACGACUGGAUGGAGAACAUCGACGUGU |
| | GCGAGAACUGCCACUACCCUAUCGUGCCUCUGGACGGCAAGGGCACCCUGCUGAUCAG |
| | AAACGGCAGCGAGGUGAGAGACCCUCUGGUGACCCUGAAGCUGCUGCAGACCAUCGGC |
| | AAGGGCGAGUUCGGCGACGUGAUGCUGGGCGACUACAGAGGCAACAAGGUGGCCGUGA |
| | AGUGCAUCAAGAACGACGCCACCGCCCAGGCCUUCCUGGCCGAGGCCAGCGUGAUGACC |
| | CAGCUGAGACACAGCAACCUGGUGCAGCUGCUGGGCGUGAUCGUGGAGGAGAAGGGCG |
| | GCCUGUACAUCGUGACCGAGUACAUGGCCAAGGGCAGCCUGGUGGACUACCUGAGAAG |
| | CAGAGGCAGAAGCGUGCUGGGCGGCGACUGCCUGCUGAAGUUCAGCCUGGACGUGUGC |
| | GAGGCCAUGGAGUACCUGGAGGGCAACAACUUCGUGCACAGAGACCUGGCCGCCAGAA |
| | ACGUGCUGGUGAGCGAGGACAACGUGGCCAAGGUGAGCGACUUCGGCCUGACCAAGGA |
| | GGCCAGCAGCACCCAGGACACCGGCAAGCUGCCUGUGAAGUGGACCGCCCCUGAGGCCC |
| | UGAGAGAGAAGAAGUUCAGCACCAAGAGCGACGUGUGGAGCUUCGGCAUCCUGCUGUG |
| | GGAGAUCUACAGCUUCGGCAGAGUGCCUUACCCUAGAAUCCCUCUGAAGGACGUGGUG |
| | CCUAGAGUGGAGAAGGGCUACAAGAUGGACGCCCCUGACGGCUGCCCUCCUGCCGUGU |
| | ACGAGGUGAUGAAGAACUGCUGGCACCUGGACGCCGCCAUGAGACCUAGCUUCCUGCA |
| | GCUGAGAGAGCAGCUGGAGCACAUCAAGACCCACGAGCUGCAC |
| | [TCD21 nt. seq.] |
| 56 | AUGGGCUGCGUGCAGUGCAAGGACAAGGAGGCCACCAAGCUGACCGAGGAGAGAGACG |
| | GCAGCCUGAACCAGAGCAGCGGCUACAGAUACGGCACCGACCCUACCCCUCAGCACUAC |
| | CCUAGCUUCGGCGUGACCAGCAUCCCUAACUACCUGAAGCUGCUGCAGACCAUCGGCA |
| | AGGGCGAGUUCGGCGACGUGAUGCUGGGCGACUACAGAGGCAACAAGGUGGCCGUGAA |
| | GUGCAUCAAGAACGACGCCACCGCCCAGGCCUUCCUGGCCGAGGCCAGCGUGAUGACCC |
| | AGCUGAGACACAGCAACCUGGUGCAGCUGCUGGGCGUGAUCGUGGAGGAGAAGGGCGG |
| | CCUGUACAUCGUGACCGAGUACAUGGCCAAGGGCAGCCUGGUGGACUACCUGAGAAGC |
| | AGAGGCAGAAGCGUGCUGGGCGGCGACUGCCUGCUGAAGUUCAGCCUGGACGUGUGCG |
| | AGGCCAUGGAGUACCUGGAGGGCAACAACUUCGUGCACAGAGACCUGGCCGCCAGAAA |
| | CGUGCUGGUGAGCGAGGACAACGUGGCCAAGGUGAGCGACUUCGGCCUGACCAAGGAG |
| | GCCAGCAGCACCCAGGACACCGGCAAGCUGCCUGUGAAGUGGACCGCCCCUGAGGCCCU |
| | GAGAGAGAAGAAGUUCAGCACCAAGAGCGACGUGUGGAGCUUCGGCAUCCUGCUGUGG |
| | GAGAUCUACAGCUUCGGCAGAGUGCCUUACCCUAGAAUCCCUCUGAAGGACGUGGUGC |
| | CUAGAGUGGAGAAGGGCUACAAGAUGGACGCCCCUGACGGCUGCCCUCCUGCCGUGUA |
| | CGAGGUGAUGAAGAACUGCUGGCACCUGGACGCCGCCAUGAGACCUAGCUUCCUGCAG |
| | CUGAGAGAGCAGCUGGAGCACAUCAAGACCCACGAGCUGCAC |
| | [TCD22 nt. seq.] |
| 57 | AUGGGCAGCAACAAGAGCAAGCCUAAGGACCUGAAGCUGCUGCAGACCAUCGGCAAGG |
| | GCGAGUUCGGCGACGUGAUGCUGGGCGACUACAGAGGCAACAAGGUGGCCGUGAAGUG |
| | CAUCAAGAACGACGCCACCGCCCAGGCCUUCCUGGCCGAGGCCAGCGUGAUGACCCAGC |
| | UGAGACACAGCAACCUGGUGCAGCUGCUGGGCGUGAUCGUGGAGGAGAAGGGCGGCCU |
| | GUACAUCGUGACCGAGUACAUGGCCAAGGGCAGCCUGGUGGACUACCUGAGAAGCAGA |
| | GGCAGAAGCGUGCUGGGCGGCGACUGCCUGCUGAAGUUCAGCCUGGACGUGUGCGAGG |
| | CCAUGGAGUACCUGGAGGGCAACAACUUCGUGCACAGAGACCUGGCCGCCAGAAACGU |
| | GCUGGUGAGCGAGGACAACGUGGCCAAGGUGAGCGACUUCGGCCUGACCAAGGAGGCC |
| | AGCAGCACCCAGGACACCGGCAAGCUGCCUGUGAAGUGGACCGCCCCUGAGGCCCUGA |
| | GAGAGAAGAAGUUCAGCACCAAGAGCGACGUGUGGAGCUUCGGCAUCCUGCUGUGGGA |
| | GAUCUACAGCUUCGGCAGAGUGCCUUACCCUAGAAUCCCUCUGAAGGACGUGGUGCCU |
| | AGAGUGGAGAAGGGCUACAAGAUGGACGCCCCUGACGGCUGCCCUCCUGCCGUGUACG |
| | AGGUGAUGAAGAACUGCUGGCACCUGGACGCCGCCAUGAGACCUAGCUUCCUGCAGCU |
| | GAGAGAGCAGCUGGAGCACAUCAAGACCCACGAGCUGCAC |
| | [TCD23 nt. seq.] |
| 58 | AUGGAGGAGGCCAUCCUGGUGCCUUGCGUGCUGGGCCUGCUGCUGCUGCCUAUCCUGG |
| | CCAUGCUGAUGGCCCUGUGCGUGCACUGCCACAGACUGCCUUUCUGGGAGGAGUUCGA |
| | GAGCCUGCAGAAGCAGGAGGUGAAGAACCUGCACCAGAGACUGGAGGGCCAGCGCCCU |
| | GAGAACAAGGGCAAGAACAGAUACAAGAACAUCCUGCCUUUCGACCACAGCAGAGUGA |
| | UCCUGCAGGGCAGAGACAGCAACAUCCCUGGCAGCGACUACAUCAACGCCAACUACAU |
| | CAAGAACCAGCUGCUGGGCCCUGACGAGAACGCCAAGACCUACAUCGCCAGCCAGGGC |
| | UGCCUGGAGGCCACCGUGAACGACUUCUGGCAGAUGGCCUGGCAGGAGAAUUCCCGCG |
| | UAAUCGUGAUGACCACCAGAGAGGUGGAGAAGGGCAGAAACAAGUGCGUGCCUUACUG |
| | GCCUGAGGUGGGCAUGCAGAGAGCCUACGGCCCUUACAGCGUGACCAACUGCGGCGAG |
| | CACGACACCACCGAGUACAAGCUGAGAACCCUGCAGGUGAGCCCUCUGGACAACGGCG |
| | ACCUGAUCAGAGAGAUCUGGCACUACCAGUACCUGAGCUGGCCUGACCACGGCGUGCC |
| | UAGCGAGCCUGGCGGCGUGCUGAGCUUCCUGGACCAGAUCAACCAGAGACAGGAGAGC |
| | CUGCCUCACGCCGGCCCUAUCAUCGUGCACUGCAGCGCCGGCAUCGGCAGAACCGGCAC |
| | CAUCAUCGUGAUCGACAUGCUGAUGGAGAACAUCAGCACCAAGGGCCUGGACUGCGAC |
| | AUCGACAUCCAGAAGACCAUCCAGAUGGUGAGAGCCCAGAGAAGCGGCAUGGUGCAGA |
| | CCGAGGCCCAGUACAAGUUCAUCUACGUGGCCAUCGCCCAGUUC |
| | [TCD24 nt. seq.] |
| 59 | AUGGGCCCUGCCGGCAGCCUGCUGGGCAGCGGCCAGAUGCAGAUCACCCUGUGGGGCA |
| | GCCUGGCCGCCGUGGCCAUCUUCUUCGUGAUCACCUUCCUGAUCUUCCUGUGCAGCAGC |
| | UGCGACAGAGAGAAGAAGCCUAGAUUCUGGGAGGAGUUCGAGAGCCUGCAGAAGCAGG |
| | AGGUGAAGAACCUGCACCAGAGACUGGAGGGCCAGCGACCUGAGAACAAGGGCAAGAA |
| | CAGAUACAAGAACAUCCUGCCUUUCGACCACAGCAGAGUGAUCCUGCAGGGCAGAGAC |
| | AGCAACAUCCCUGGCAGCGACUACAUCAACGCCAAUUACAUCAAGAACCAGCUGCUGG |
| | GCCCUGACGAGAACGCCAAGACCUACAUCGCCAGCCAGGGCUGCCUGGAGGCCACCGUG |
| | AACGACUUCUGGCAGAUGGCCUGGCAGGAGAACAGUCGCGUGAUCGUGAUGACCACCA |
| | GAGAGGUGGAGAAGGGCAGAAACAAGUGCGUGCCUUACUGGCCUGAGGUGGGCAUGCA |
| | GAGAGCCUACGGCCCUUACAGCGUGACCAACUGCGGCGAGCACGACACCACCGAGUAC |
| | AAGCUGAGAACCCUGCAGGUGAGCCCUCUGGACAACGGCGACCUGAUCAGAGAGAUCU |
| | GGCACUACCAGUACCUGAGCUGGCCUGACCACGGCGUGCCUAGCGAGCCUGGCGGCGU |
| | GCUGAGCUUCCUGGACCAGAUCAACCAGAGACAGGAAAGUCUGCCUCACGCCGGCCCU |
| | AUCAUCGUGCACUGCAGCGCCGGCAUCGGCAGAACCGGCACCAUCAUCGUGAUCGACA |
| | UGCUGAUGGAGAACAUCAGCACCAAGGGCCUGGACUGCGACAUCGACAUCCAGAAGAC |
| | CAUCCAGAUGGUGAGAGCCCAGAGAAGCGGCAUGGUGCAGACCGAGGCCCAGUACAAG |
| | UUCAUCUACGUAGCAAUCGCCCAGUUC |
| | [TCD25 nt. seq.] |
| 60 | AUGGGCUGCGGCUGCAGCAGCCACCCUGAGGACGACUGGAUGGAGAACAUCGACGUGU |
| | GCGAGAACUGCCACUACCCUAUCGUGCCUCUGGACGGCAAGGGCACCCUGCUGAUCAG |
| | AAACGGCAGCGAGGUGAGGGACCCUCUGGUGACCUUCUGGGAGGAGUUCGAGAGCCUG |
| | CAGAAGCAGGAGGUGAAGAACCUGCACCAGAGACUGGAGGGCCAAAGGCCUGAGAACA |
| | AGGGCAAGAACAGAUACAAGAACAUCCUGCCUUUCGACCACAGCAGAGUGAUCCUGCA |
| | GGGCAGAGACAGCAACAUCCCUGGCAGCGACUACAUCAACGCCAACUACAUCAAGAAC |
| | CAGCUGCUGGGCCCUGACGAGAACGCCAAGACCUACAUCGCCAGCCAGGGCUGCCUGG |
| | AGGCCACCGUGAACGACUUCUGGCAGAUGGCCUGGCAGGAGAACUCCAGGGUCAUUGU |
| | GAUGACCACCAGAGAGGUGGAGAAGGGCAGAAACAAGUGCGUGCCUUACUGGCCUGAG |
| | GUGGGCAUGCAGAGAGCCUACGGCCCUUACAGCGUGACCAACUGCGGCGAGCACGACA |
| | CCACCGAGUACAAGCUGAGAACCCUGCAGGUGAGCCCUCUGGACAACGGCGACCUGAU |
| | CAGAGAGAUCUGGCACUACCAGUACCUGAGCUGGCCUGACCACGGCGUGCCUAGCGAG |
| | CCUGGCGGCGUGCUGAGCUUCCUGGACCAGAUCAACCAGAGACAGGAGAGCCUGCCUC |
| | ACGCCGGCCCUAUCAUCGUGCACUGCAGCGCCGGCAUCGGCAGAACCGGCACCAUCAUC |
| | GUGAUCGACAUGCUGAUGGAGAAUAUCAGCACCAAGGGCCUGGACUGCGACAUCGACA |
| | UCCAGAAGACCAUCCAGAUGGUGAGAGCCCAGAGAAGCGGCAUGGUGCAGACCGAGGC |
| | CCAGUACAAGUUCAUCUACGUGGCCAUCGCCCAGUUC |
| | [TCD26 nt. seq.] |
| 61 | AUGGGCUGCGUGCAGUGCAAGGACAAGGAGGCCACCAAGCUGACCGAGGAGAGAGACG |
| | GCAGCCUGAACCAGAGCAGCGGCUACAGAUACGGCACCGACCCUACCCCUCAGCACUAC |
| | CCUAGCUUCGGCGUGACCAGCAUCCCUAACUACUUCUGGGAGGAGUUCGAGAGCCUGC |
| | AGAAGCAGGAGGUGAAGAACCUGCACCAGAGACUGGAGGGCCAGCGGCCUGAGAACAA |
| | GGGCAAGAACAGAUACAAGAACAUCCUGCCUUUCGACCACAGCAGAGUGAUCCUGCAG |
| | GGCAGAGACAGCAACAUCCCUGGCAGCGACUACAUCAACGCCAACUACAUCAAGAACC |
| | AGCUGCUGGGCCCUGACGAGAACGCCAAGACCUACAUCGCCAGCCAGGGCUGCCUGGA |
| | GGCCACCGUGAACGACUUCUGGCAGAUGGCCUGGCAGGAGAACAGUCGUGUGAUCGUG |
| | AUGACCACCAGAGAGGUGGAGAAGGGCAGAAACAAGUGCGUGCCUUACUGGCCUGAGG |
| | UGGGCAUGCAGAGAGCCUACGGCCCUUACAGCGUGACCAACUGCGGCGAGCACGACAC |
| | CACCGAGUACAAGCUGAGAACCCUGCAGGUGAGCCCUCUGGACAACGGCGACCUGAUC |
| | AGAGAGAUCUGGCACUACCAGUACCUGAGCUGGCCUGACCACGGCGUGCCUAGCGAGC |
| | CUGGCGGCGUGCUGAGCUUCCUGGACCAGAUCAACCAGAGACAGGAGAGCCUGCCUCA |
| | CGCCGGCCCUAUCAUCGUGCACUGCAGCGCCGGCAUCGGCAGAACCGGCACCAUCAUCG |
| | UGAUCGACAUGCUGAUGGAGAACAUCAGCACCAAGGGCCUGGACUGCGACAUCGACAU |
| | CCAGAAGACCAUCCAGAUGGUGAGAGCCCAGAGAAGCGGCAUGGUGCAGACCGAGGCC |
| | CAGUACAAGUUCAUCUACGUGGCCAUCGCCCAGUUC |
| | [TCD27 nt. seq.] |
| 62 | AUGGGCAGCAACAAGAGCAAGCCUAAGGACUUCUGGGAGGAGUUCGAGAGCCUGCAGA |
| | AGCAGGAGGUGAAGAACCUGCACCAGAGACUGGAGGGCCAACGCCCUGAGAACAAGGG |
| | CAAGAACAGAUACAAGAACAUCCUGCCUUUCGACCACAGCAGAGUGAUCCUGCAGGGC |
| | AGAGACAGCAACAUCCCUGGCAGCGACUACAUCAACGCCAACUACAUCAAGAACCAGC |
| | UGCUGGGCCCUGACGAGAACGCCAAGACCUACAUCGCCAGCCAGGGCUGCCUGGAGGC |
| | CACCGUGAACGACUUCUGGCAGAUGGCCUGGCAGGAGAAUAGCAGGGUUAUUGUGAUG |
| | ACCACCAGAGAGGUGGAGAAGGGCAGAAACAAGUGCGUGCCUUACUGGCCUGAGGUGG |
| | GCAUGCAGAGAGCCUACGGCCCUUACAGCGUGACCAACUGCGGCGAGCACGACACCACC |
| | GAGUACAAGCUGAGAACCCUGCAGGUGAGCCCUCUGGACAACGGCGACCUGAUCAGAG |
| | AGAUCUGGCACUACCAGUACCUGAGCUGGCCUGACCACGGCGUGCCUAGCGAGCCUGG |
| | CGGCGUGCUGAGCUUCCUGGACCAGAUCAACCAGAGACAGGAGAGCCUGCCUCACGCC |
| | GGCCCUAUCAUCGUGCACUGCAGCGCCGGCAUCGGCAGAACCGGCACCAUCAUCGUGA |
| | UCGACAUGCUGAUGGAGAACAUCAGCACCAAGGGCCUGGACUGCGACAUCGACAUCCA |
| | GAAGACCAUCCAGAUGGUGAGAGCCCAGAGAAGCGGCAUGGUGCAGACCGAGGCCCAG |
| | UACAAGUUCAUCUACGUGGCCAUCGCCCAGUUC |
| | [TCD28 nt. seq.] |
| 63 | AUGGAGGAGGCCAUCCUGGUGCCUUGCGUGCUGGGCCUGCUGCUCCUUCCUAUCCUGG |
| | CCAUGCUGAUGGCCCUGUGCGUGCACUGCCACAGACUGCCUGUGCGGUGGUUCCACAG |
| | AGAUCUGAGCGGCCUGGACGCCGAGACACUGCUGAAGGGCAGAGGCGUGCACGGCAGC |
| | UUCCUUGCAAGACCUAGCAGAAAGAACCAGGGCGACUUCAGCCUGAGCGUGAGAGUGG |
| | GCGACCAGGUGACCCACAUCAGAAUCCAGAACAGCGGAGAUUUCUACGACCUGUACGG |
| | CGGCGAGAAGUUCGCCACCCUGACCGAGCUGGUGGAGUACUACACCCAGCAGCAGGGC |
| | GUGCUGCAGGACAGAGACGGCACCAUCAUCCACCUGAAGUACCCUCUGAACUGCAGCG |
| | ACCCUACCAGCGAGCGCUGGUACCACGGCCACAUGAGCGGCGGCCAGGCAGAGACACUC |
| | CUCCAGGCCAAGGGCGAGCCUUGGACCUUCCUGGUGAGAGAGAGUCUAUCCCAGCCUG |
| | GUGACUUCGUGUUGAGCGUACUCUCGGACCAGCCUAAGGCCGGCCCUGGCAGCCCUCU |
| | GAGAGUCACACACAUUAAGGUGAUGUGCGAGGGCGGCAGAUACACCGUGGGAGGCCUU |
| | GAGACUUUCGACUCACUGACAGACCUCGUGGAGCACUUCAAGAAGACCGGCAUCGAAG |
| | AGGCAAGCGGCGCCUUCGUGUACCUGAGACAGCCUUACUACGCCACCAGAGUGAACGC |
| | CGCCGACAUCGAGAACAGAGUGCUGGAGCUGAACAAGAAGCAGGAGAGCGAGGACACC |
| | GCCAAGGCGGGAUUCUGGGAGGAGUUCGAAUCUCUGCAGAAGCAAGAAGUGAAGAACC |
| | UGCACCAGAGACUGGAGGGCCAGCGUCCAGAGAACAAGGGCAAGAACAGAUACAAGAA |
| | CAUCCUGCCUUUCGACCACAGCAGAGUGAUCCUGCAGGGCAGGGACAGCAACAUCCCU |
| | GGAAGUGACUACAUCAACGCCAAUUACAUUAAGAAUCAGCUGCUGGGACCUGACGAGA |
| | ACGCUAAGACGUACAUCGCCAGCCAGGGCUGCCUGGAGGCCACCGUGAACGACUUCUG |
| | GCAGAUGGCCUGGCAGGAGAAUUCACGUGUGAUUGUGAUGACAACCCGAGAGGUGGAG |
| | AAGGGCCGUAACAAGUGCGUGCCUUACUGGCCUGAGGUGGGCAUGCAGAGAGCCUACG |
| | GCCCUUACAGCGUGACCAACUGCGGCGAGCACGACACCACCGAGUACAAGCUGAGAAC |
| | CCUGCAGGUGUCACCUCUUGACAACGGCGACCUGAUCAGAGAGAUCUGGCACUACCAA |
| | UAUUUAUCUUGGCCAGACCACGGCGUGCCUAGCGAGCCAGGCGGUGUCCUUAGCUUCC |
| | UGGACCAGAUCAAUCAGCGCCAAGAGUCCCUCCCUCACGCUGGCCCAAUCAUCGUUCAC |
| | UGCUCUGCAGGCAUCGGCAGAACUGGUACAAUUAUCGUCAUCGAUAUGUUAAUGGAGA |
| | ACAUCAGCACCAAGGGUCUGGACUGUGACAUUGACAUCCAGAAGACCAUCCAGAUGGU |
| | UAGGGCCCAGAGAAGCGGCAUGGUGCAGACCGAGGCCCAGUACAAGUUCAUCUACGUG |
| | GCCAUCGCCCAGUUC |
| | [TCD29 nt. seq.] |
| 64 | AUGGGCCCUGCCGGCAGCCUGCUGGGCAGCGGCCAGAUGCAGAUCACCCUGUGGGGUU |
| | CGUUGGCCGCCGUGGCCAUCUUCUUCGUGAUCACCUUCCUGAUCUUCCUGUGCAGCAG |
| | CUGCGACAGAGAGAAGAAGCCUAGAGUGCGCUGGUUCCACCGCGACCUGAGCGGCCUG |
| | GACGCCGAGACACUGCUGAAGGGCAGAGGCGUGCACGGCAGCUUCCUGGCCAGACCUA |
| | GCAGAAAGAACCAGGGCGACUUCAGCCUGAGCGUGAGAGUGGGCGACCAGGUGACCCA |
| | CAUCAGAAUCCAGAACAGCGGCGAUUUCUACGACCUGUACGGCGGCGAGAAGUUCGCC |
| | ACCCUGACCGAGCUGGUGGAGUACUACACCCAGCAGCAGGGCGUGCUGCAGGACAGAG |
| | ACGGCACCAUCAUCCACCUGAAGUACCCUCUGAACUGCAGCGACCCUACCAGCGAGCGG |
| | UGGUACCACGGCCACAUGAGCGGCGGCCAGGCCGAGACUCUCCUUCAGGCCAAGGGCG |
| | AGCCUUGGACAUUCCUCGUGAGAGAGUCGCUGUCACAGCCUGGUGAUUUCGUGCUGAG |
| | UGUACUGAGCGACCAGCCUAAGGCCGGCCCUGGCAGCCCUCUGAGAGUCACUCACAUC |
| | AAGGUGAUGUGCGAGGGCGGCAGAUACACCGUGGGAGGCUUAGAGACUUUCGACUCCU |
| | UAACCGACCUAGUGGAACACUUCAAGAAGACCGGCAUCGAGGAGGCCAGCGGCGCCUU |
| | CGUGUACCUGAGACAGCCUUACUACGCCACCAGAGUGAACGCCGCCGACAUCGAGAAC |
| | AGAGUGCUGGAGCUGAACAAGAAGCAGGAGAGCGAGGACACCGCCAAGGCAGGCUUCU |
| | GGGAGGAGUUCGAGAGUCUGCAGAAGCAGGAAGUGAAGAACCUGCACCAGAGACUGGA |
| | GGGCCAACGUCCGGAGAACAAGGGCAAGAACAGAUACAAGAACAUCCUGCCUUUCGAC |
| | CACAGCAGAGUGAUCCUGCAAGGAAGGGACAGCAACAUCCCAGGAUCAGACUACAUCA |
| | ACGCCAAUUAUAUCAAGAACCAACUGUUAGGACCUGACGAGAACGCCAAGACCUACAU |
| | CGCCAGCCAGGGCUGCCUGGAGGCCACCGUGAACGACUUCUGGCAGAUGGCCUGGCAG |
| | GAGAACUCGCGUGUUAUUGUGAUGACUACUCGAGAGGUGGAGAAGGGACGGAACAAGU |
| | GCGUGCCUUACUGGCCUGAGGUGGGCAUGCAGCGGGCUUACGGCCCUUACAGCGUGAC |
| | CAACUGCGGCGAGCACGACACCACCGAGUACAAGCUGAGAACCCUGCAGGUGAGUCCU |
| | CUGGACAACGGCGACCUGAUCAGAGAGAUCUGGCACUACCAGUACCUCAGUUGGCCUG |
| | ACCACGGCGUGCCUAGCGAACCAGGCGGCGUACUGUCCUUCCUGGACCAGAUCAAUCA |
| | GCGCCAAGAAUCUCUUCCUCACGCCGGACCGAUCAUCGUGCACUGCAGUGCCGGUAUU |
| | GGCAGAACAGGCACCAUUAUUGUCAUCGACAUGCUGAUGGAGAACAUCAGCACCAAGG |
| | GACUUGAUUGUGAUAUAGACAUCCAGAAGACCAUCCAGAUGGUUAGAGCCCAGAGAAG |
| | CGGCAUGGUGCAGACCGAGGCCCAGUACAAGUUCAUCUACGUAGCCAUAGCCCAGUUC |
| | [TCD30 nt. seq.] |
| 65 | AUGGGCUGCGGCUGCAGCAGCCACCCUGAGGACGACUGGAUGGAGAACAUCGACGUGU |
| | GCGAGAACUGCCACUACCCUAUCGUGCCUCUGGACGGCAAGGGCACCCUGCUGAUCAG |
| | AAACGGCAGCGAGGUGCGUGACCCUCUGGUGACCGUGCGCUGGUUCCACAGGGACCUG |
| | AGCGGCCUGGACGCCGAAACGUUGCUCAAGGGCAGAGGCGUGCACGGCAGCUUCCUGG |
| | CCAGACCUAGCAGAAAGAACCAGGGCGACUUCAGCCUGAGCGUGAGAGUGGGCGACCA |
| | GGUGACCCACAUCAGAAUCCAGAACAGCGGCGACUUCUACGACCUGUACGGCGGCGAG |
| | AAGUUCGCUACUCUCACCGAGCUGGUGGAGUACUACACCCAGCAGCAGGGCGUGCUGC |
| | AGGACAGAGACGGCACCAUCAUCCACCUGAAGUACCCUCUGAACUGCAGCGACCCUACC |
| | AGCGAGCGAUGGUACCACGGCCACAUGAGCGGCGGCCAGGCCGAGACACUACUGCAGG |
| | CCAAGGGCGAGCCUUGGACCUUCCUGGUGAGAGAGAGCCUGAGCCAGCCUGGCGACUU |
| | CGUGCUGAGUGUGCUUAGCGACCAGCCUAAGGCCGGCCCUGGCAGCCCUCUGAGAGUC |
| | ACACAUAUCAAGGUGAUGUGCGAGGGCGGCAGAUACACCGUGGGAGGUUUGGAGACUU |
| | UCGACAGCCUGACCGACCUGGUGGAGCACUUCAAGAAGACCGGCAUCGAGGAGGCCAG |
| | CGGCGCCUUCGUGUACCUGAGACAGCCUUACUACGCCACCAGAGUGAACGCCGCCGACA |
| | UCGAGAACAGAGUGCUGGAGCUGAACAAGAAGCAGGAGAGCGAGGACACCGCCAAGGC |
| | CGGCUUCUGGGAGGAGUUCGAGAGCCUGCAGAAGCAAGAGGUGAAGAACCUGCACCAG |
| | AGACUGGAGGGACAGCGACCGGAGAACAAGGGCAAGAACAGAUACAAGAACAUCCUGC |
| | CUUUCGACCACAGCAGAGUGAUCCUGCAGGGCAGAGACAGCAACAUCCCAGGAAGCGA |
| | CUACAUCAACGCCAAUUAUAUCAAGAACCAGCUGCUGGGCCCUGACGAGAACGCCAAG |
| | ACCUACAUCGCCAGCCAGGGCUGCCUGGAGGCCACCGUGAACGACUUCUGGCAGAUGG |
| | CCUGGCAGGAGAACUCUCGGGUUAUCGUGAUGACCACCAGAGAGGUGGAGAAGGGUAG |
| | AAACAAGUGCGUGCCUUACUGGCCUGAGGUGGGCAUGCAGAGAGCCUACGGCCCUUAC |
| | AGCGUGACCAACUGCGGCGAGCACGACACCACCGAGUACAAGCUGAGAACCCUGCAGG |
| | UGAGCCCUCUGGACAACGGCGACCUGAUCAGAGAGAUCUGGCACUACCAGUACCUGAG |
| | CUGGCCUGACCACGGCGUGCCUAGCGAGCCUGGCGGAGUGUUGUCGUUCCUGGACCAG |
| | AUCAACCAGAGACAGGAAAGUUUACCUCACGCCGGCCCUAUCAUCGUGCACUGCAGCG |
| | CCGGCAUCGGCAGAACUGGCACUAUAAUCGUGAUCGACAUGUUAAUGGAGAAUAUCAG |
| | CACCAAGGGCCUGGACUGCGACAUCGACAUCCAGAAGACCAUCCAGAUGGUGAGAGCC |
| | CAGAGAAGCGGCAUGGUGCAGACCGAGGCCCAGUACAAGUUCAUCUACGUGGCCAUCG |
| | CCCAGUUC |
| | [TCD31 nt. seq.] |
| 66 | AUGGGCUGCGUGCAGUGCAAGGACAAGGAGGCCACCAAGCUGACCGAGGAGAGAGACG |
| | GCAGCCUGAACCAGAGCAGCGGCUACAGAUACGGCACCGACCCUACCCCUCAGCACUAC |
| | CCUAGCUUCGGCGUGACCAGCAUCCCUAACUACGUGCGGUGGUUCCACAGGGAUCUGA |
| | GCGGCCUGGACGCCGAAACCCUGCUGAAGGGCAGAGGCGUGCACGGCAGCUUCCUGGC |
| | CAGACCUAGCAGAAAGAACCAGGGCGACUUCAGCCUGAGCGUGAGAGUGGGCGACCAG |
| | GUGACCCACAUCAGAAUCCAGAACAGCGGAGACUUCUACGACCUGUACGGCGGCGAGA |
| | AGUUCGCCACCCUCACAGAACUGGUGGAGUACUACACCCAGCAGCAGGGCGUGCUGCA |
| | GGACAGGGACGGAACCAUCAUCCACCUGAAGUACCCUCUGAACUGCAGCGAUCCAACA |
| | AGCGAGCGGUGGUACCACGGCCACAUGAGCGGCGGCCAGGCUGAGACAUUACUCCAGG |
| | CCAAGGGCGAGCCUUGGACCUUCCUGGUGAGAGAGUCCUUGAGCCAGCCUGGUGAUUU |
| | CGUGCUGAGUGUGCUCUCUGACCAGCCUAAGGCCGGCCCUGGCAGCCCUCUGAGAGUU |
| | ACUCAUAUCAAGGUGAUGUGCGAGGGCGGCAGAUACACCGUGGGUGGCCUCGAGACAU |
| | UCGACAGCCUGACCGACCUGGUGGAACACUUCAAGAAGACCGGCAUCGAGGAAGCAAG |
| | CGGCGCCUUCGUGUACCUGAGACAGCCUUACUACGCCACCAGAGUGAACGCCGCCGACA |
| | UCGAGAACAGAGUGCUGGAGCUGAACAAGAAGCAGGAGAGCGAGGACACCGCCAAGGC |
| | AGGUUUCUGGGAGGAGUUCGAAAGCCUGCAGAAGCAAGAAGUGAAGAACCUGCACCAG |
| | AGACUGGAGGGCCAACGGCCAGAGAACAAGGGCAAGAACAGAUACAAGAACAUCCUGC |
| | CUUUCGACCACAGCAGAGUGAUCCUUCAGGGCCGAGACAGCAACAUCCCUGGCUCAGA |
| | CUACAUCAACGCCAAUUACAUUAAGAAUCAGCUGCUGGGCCCUGACGAGAACGCCAAG |
| | ACCUACAUCGCCAGCCAGGGCUGCCUCGAAGCCACGGUGAACGACUUCUGGCAGAUGG |
| | CCUGGCAGGAGAAUAGCCGGGUGAUCGUGAUGACAACCAGAGAGGUGGAGAAGGGUAG |
| | AAACAAGUGCGUGCCUUACUGGCCUGAGGUGGGCAUGCAGCGGGCCUACGGCCCUUAC |
| | AGUGUGACGAACUGCGGCGAGCACGACACCACCGAGUACAAGCUGAGAACCCUGCAGG |
| | UGUCACCACUGGACAACGGCGACCUGAUCAGAGAGAUCUGGCACUACCAGUAUCUUAG |
| | UUGGCCGGACCACGGCGUGCCUAGCGAGCCUGGCGGCGUCCUGAGCUUCUUGGACCAG |
| | AUCAAUCAGAGACAGGAGUCCCUGCCUCACGCGGGCCCGAUCAUCGUGCAUUGUUCUG |
| | CAGGCAUCGGCAGAACCGGCACUAUCAUCGUCAUCGACAUGCUGAUGGAGAACAUCAG |
| | CACCAAGGGUCUGGACUGCGACAUAGACAUCCAGAAGACCAUCCAGAUGGUGCGGGCC |
| | CAGAGAAGCGGCAUGGUGCAGACCGAGGCCCAGUACAAGUUCAUCUACGUGGCCAUCG |
| | CCCAGUUC |
| | [TCD32 nt. seq.] |
| 67 | AUGGGCAGCAACAAGAGCAAGCCUAAGGACGUGCGCUGGUUCCACCGCGACCUGAGCG |
| | GCCUGGACGCCGAGACACUGCUGAAGGGCAGAGGCGUGCACGGCAGCUUCCUGGCCAG |
| | ACCUAGCAGAAAGAACCAGGGCGACUUCAGCCUGAGCGUGAGAGUGGGCGACCAGGUG |
| | ACCCACAUCAGAAUCCAGAACAGCGGAGAUUUCUACGACCUGUACGGCGGCGAGAAGU |
| | UCGCCACCCUGACCGAGCUGGUGGAGUACUACACCCAGCAGCAGGGCGUGCUGCAGGA |
| | CAGAGACGGCACCAUCAUCCACCUGAAGUACCCUCUGAACUGCAGCGACCCUACCAGCG |
| | AGCGGUGGUACCACGGCCACAUGAGCGGCGGCCAGGCUGAAACACUCCUCCAAGCCAA |
| | GGGCGAGCCUUGGACCUUCCUGGUGAGAGAGAGCCUGUCUCAGCCUGGUGACUUCGUG |
| | CUGUCAGUUCUGUCCGACCAGCCAAAGGCCGGCCCUGGCAGCCCUCUGAGAGUCACCCA |
| | UAUAAAGGUGAUGUGCGAGGGCGGCAGAUACACCGUGGGAGGCUUGGAAACAUUCGAC |
| | AGUCUAACAGACCUUGUCGAACACUUCAAGAAGACCGGCAUCGAGGAGGCCAGCGGCG |
| | CCUUCGUGUACCUGAGACAGCCUUACUACGCCACCAGAGUGAACGCCGCCGACAUCGA |
| | GAACAGAGUGCUGGAGCUGAACAAGAAGCAGGAGAGCGAGGACACCGCCAAGGCGGGA |
| | UUCUGGGAGGAGUUCGAAUCCUUACAGAAGCAGGAAGUGAAGAACCUGCACCAGAGAC |
| | UGGAGGGCCAGAGGCCAGAGAACAAGGGCAAGAACAGAUACAAGAACAUCCUGCCUUU |
| | CGACCACAGCAGAGUGAUCCUGCAAGGCAGGGACAGCAACAUUCCAGGCUCAGACUAC |
| | AUCAACGCCAACUAUAUCAAGAAUCAGCUGCUGGGCCCUGACGAGAACGCCAAGACCU |
| | ACAUCGCCAGCCAGGGCUGCCUGGAGGCCACCGUGAACGACUUCUGGCAGAUGGCCUG |
| | GCAGGAGAACUCAAGAGUCAUCGUGAUGACAACUCGGGAGGUGGAGAAGGGACGAAAC |
| | AAGUGCGUGCCUUACUGGCCUGAGGUGGGCAUGCAGCGAGCUUACGGCCCUUACAGCG |
| | UGACCAACUGCGGCGAGCACGACACCACCGAGUACAAGCUGAGAACCCUGCAGGUGUC |
| | GCCACUGGACAACGGCGACCUGAUCAGAGAGAUCUGGCACUACCAAUAUUUAUCUUGG |
| | CCGGAUCACGGCGUGCCUAGCGAGCCUGGCGGUGUGUUGAGUUUCCUGGACCAGAUCA |
| | AUCAGCGGCAGGAGUCAUUACCUCACGCCGGACCAAUCAUCGUGCACUGCUCAGCCGG |
| | AAUUGGCAGAACAGGAACCAUUAUCGUGAUCGACAUGCUGAUGGAGAACAUCAGCACC |
| | AAGGGACUGGACUGUGAUAUAGACAUCCAGAAGACCAUCCAGAUGGUGCGCGCCCAGA |
| | GAAGCGGCAUGGUGCAGACCGAGGCCCAGUACAAGUUCAUCUACGUGGCCAUCGCCCA |
| | GUUC |
| | [TCD33 nt. seq.] |
| 68 | AUGGAGGCCGACGCCCUGAGCCCUGUGGGCCUGGGCCUGCUGCUGCUGCCUUUCCUGG |
| | UGACCCUGCUGGCCGCCCUGUGCGUGAGAUGCAGAGAGCUGCCUGUGAGCUACGACGC |
| | CGUGAGCCUGAGCAAGAUGCUGAAGAAGAGAAGCCCUCUGACCACCGGCGUGUACGUG |
| | AAGAUGCCUCCUACCGAGCCUGAGUGCGAGAAGCAGUUCCAGCCUUACUUCAUCCCUA |
| | UCAAC |
| | [TCD34 nt. seq.] |
| 69 | AUGGAGGAGGCCAUCCUGGUGCCUUGCGUGCUGGGCCUGCUGCUGCUGCCUAUCCUGG |
| | CCAUGCUGAUGGCCCUGUGCGUGCACUGCCACAGACUGCCUGGCAGCUACGACAGCGG |
| | CUUCGGCGGCGGCGGCAGCACCGCCGUGAGCCUGAGCAAGAUGCUGAAGAAGAGAAGC |
| | CCUCUGACCACCGGCGUGUACGUGAAGAUGCCUCCUACCGAGCCUGAGUGCGAGAAGC |
| | AGUUCCAGCCUUACUUCAUCCCUAUCAAC |
| | [TCD35 nt. seq.] |
| 70 | AUGGAGGAGGCCAUCCUGGUGCCUUGCGUGCUGGGCCUGCUGCUGCUGCCUAUCCUGG |
| | CCAUGCUGAUGGCCCUGUGCGUGCACUGCCACAGACUGCCUGGCAGCUACGACAGCGG |
| | CUUCGGCGGCGGCGGCAGCAUGGAGAAGGAGUUCGAGCAGAUCGACAAGAGCGGCAGC |
| | UGGGCCGCCAUCUACCAGGACAUCAGACACGAGGCCAGCGACUUCCCUUGCAGAGUGG |
| | CCAAGCUGCCUAAGAACAAGAACAGAAACAGAUACAGAGACGUGAGCCCUUUCGACCA |
| | CAGCAGAAUCAAGCUGCACCAGGAGGACAACGACUACAUCAACGCCAGCCUGAUCAAG |
| | AUGGAGGAGGCCCAGAGAAGCUACAUCCUGACCCAGGGCCCUCUGCCUAACACCUGCG |
| | GCCACUUCUGGGAGAUGGUGUGGGAGCAGAAGAGCAGAGGCGUGGUGAUGCUGAACAG |
| | AGUGAUGGAGAAGGGCAGCCUGAAGUGCGCCCAGUACUGGCCUCAGAAGGAGGAGAAG |
| | GAGAUGAUCUUCGAGGACACCAACCUGAAGCUGACCCUGAUCAGCGAGGACAUCAAGA |
| | GCUACUACACCGUGAGACAGCUGGAGCUGGAGAACCUGACCACCCAGGAGACCAGAGA |
| | GAUCCUGCACUUCCACUACACCACCUGGCCUGACUUCGGCGUGCCUGAGAGCCCUGCCA |
| | GCUUCCUGAACUUCCUGUUCAAGGUGAGAGAGAGCGGCAGCCUGAGCCCUGAGCACGG |
| | CCCUGUGGUGGUGCACUGCAGCGCCGGCAUCGGCAGAAGCGGCACCUUCUGCCUGGCC |
| | GACACCUGCCUGCUGCUGAUGGACAAGAGAAAGGACCCUAGCAGCGUGGACAUCAAGA |
| | AGGUGCUGCUGGAGAUGAGAAAGUUCAGAAUGGGCCUGAUCCAGACCGCCGACCAGCU |
| | GAGAUUCAGCUACCUGGCCGUGAUCGAGGGCGGCAAGCCUAGCACC |
| | [TCD36 nt. seq.] |
| 71 | AUGAGCGCCGAGGGCUACCAGUACAGAGCCCUGUACGACUACAAGAAGGAGAGAGAGG |
| | AGGACAUCGACCUGCACCUGGGCGACAUCCUGACCGUGAACAAGGGCAGCCUGGUGGC |
| | CCUGGGCUUCAGCGACGGCCAGGAGGCCAGACCUGAGGAGAUCGGCUGGCUGAACGGC |
| | UACAACGAGACAACCGGCGAGAGAGGCGACUUCCCUGGCACCUACGUGGAGUACAUCG |
| | GCAGAAAGAAGAUCAGCCCUCCUACCCCUAAGCCUAGACCUCCGCGGCCACUGCCUGUG |
| | GCCCCUGGCAGCAGCAAGACCGAGGCCGACGUGGAGCAGCAGCCUGCCCCUGCCCUGCC |
| | UCCAAAGCCUCCUAAGCCGACCACCGUGGCCAACAACGGCAUGAACAACAACAUGAGCC |
| | UGCAGGACGCCGAGUGGUACUGGGGUGACAUUAGCCGCGAGGAAGUGAACGAGAAGCU |
| | GAGAGACACCGCCGACGGCACCUUCCUGGUGAGAGACGCCAGCACCAAGAUGCACGGC |
| | GACUACACCCUGACCCUGAGAAAGGGCGGCAACAACAAGCUGAUCAAGAUCUUCCACA |
| | GAGACGGCAAGUACGGCUUCUCUGAUCCUCUGACCUUCAGCAGCGUGGUGGAGUUGAU |
| | AAACCACUACAGAAACGAGAGCCUGGCCCAGUACAACCCUAAGCUGGACGUGAAGCUG |
| | CUGUACCCUGUGAGCAAGUACCAGCAGGACCAGGUGGUGAAGGAAGACAACAUCGAGG |
| | CCGUGGGCAAGAAGCUGCACGAGUACAACACCCAGUUCCAGGAGAAGAGUCGCGAAUA |
| | CGACAGACUGUACGAGGAGUACACCAGAACCAGCCAGGAGAUCCAGAUGAAGAGAACC |
| | GCUAUAGAGGCGUUCAACGAGACUAUCAAGAUUUUCGAGGAGCAGUGCCAGACCCAGG |
| | AGAGAUACAGCAAGGAAUACAUAGAGAAGUUCAAGAGAGAAGGCAACGAGAAGGAAA |
| | UCCAGAGAAUCAUGCACAACUACGACAAGCUUAAGAGCAGAAUCAGCGAGAUCAUCGA |
| | CAGCAGAAGAAGACUGGAGGAAGACCUGAAGAAGCAGGCCGCCGAAUAUCGGGAGAUC |
| | GACAAGAGAAUGAACAGCAUCAAGCCUGACCUGAUCCAGCUGCGAAAGACCCGAGAUC |
| | AGUACCUGAUGUGGCUGACCCAGAAGGGCGUGAGACAGAAGAAGCUUAACGAGUGGUU |
| | GGGUAACGAGAACACCGAGGAUCAGUAUAGCUUAGUGGAGGACGACGAGGAUCUCCCU |
| | CACCACGACGAGAAGACUUGGAACGUGGGCAGUUCUAACAGAAACAAGGCCGAGAACC |
| | UGCUGAGAGGCAAGCGGGACGGUACAUUCCUCGUACGGGAAAGCAGUAAGCAGGGCUG |
| | CUACGCCUGUUCCGUUGUCGUGGACGGCGAGGUGAAGCACUGCGUGAUCAACAAGACC |
| | GCCACCGGCUACGGUUUCGCCGAGCCUUACAACCUGUACAGCAGUCUCAAGGAGCUGG |
| | UGCUGCACUACCAGCACACCAGCCUCGUCCAGCACAACGACUCCUUGAACGUGACCCUG |
| | GCCUAUCCAGUGUACGCCCAACAGAGGAGAGGCGGCGGUGGCUCUGGUGGAGGAGGUA |
| | GUACAGCGAUCAUCAAGGAGAUUGUGUCACGGAACGAGAGAAGAUACCAGGAGGACGG |
| | CUUCGACCUGGACCUGACCUACAUCUACCCUAACAUCAUCGCCAUGGGCUUCCCUGCCG |
| | AGCGUCUCGAGGGCGUGUAUAGAAACAAUAUUGACGACGUGGUGAGAUUCCUGGACUC |
| | UAAGCACAAGAACCACUAUAAGAUCUAUAAUCUCUGCGCCGAGAGGCAUUACGAUACC |
| | GCUAAGUUCAACUGCAGAGUAGCUCAGUAUCCAUUCGAGGACCACAACCCUCCUCAGC |
| | UGGAACUCAUUAAGCCAUUCUGCGAAGAUUUGGAUCAGUGGCUGAGCGAGGACGAUAA |
| | CCACGUGGCCGCCAUCCACUGCAAGGCCGGCAAGGGAAGAACCGGCGUGAUGAUCUGC |
| | GCCUACCUGCUGCACCGAGGCAAGUUCCUGAAGGCGCAGGAAGCUCUGGACUUCUACG |
| | GAGAAGUCAGAACUCGGGAUAAGAAGGGAGUAACCAUCCCUAGCCAGCGUAGGUACGU |
| | GUACUACUACUCAUACCUGUUGAAGAACCAUCUGGACUACAGACCUGUGGCACUGCUG |
| | UUCCACAAGAUGAUGUUCGAAACGAUUCCGAUGUUCAGUGGCGGCACCUGCAACCCUC |
| | AGUUCGUGGUGUGCCAGCUGAAGGUGAAGAUUUACUCAAGCAACAGCGGCCCUACCAG |
| | AAGAGAGGACAAGUUCAUGUACUUCGAGUUCCCUCAGCCUCUCCCAGUUUGCGGAGAC |
| | AUAAAGGUCGAGUUCUUCCAUAAGCAGAACAAGAUGUUAAAGAAGGAUAAGAUGUUCC |
| | ACUUCUGGGUGAACACCUUCUUCAUCCCUGGUCCGGAGGAGACAUCAGAGGAGGUUGA |
| | GAACGGAAGUCUCUGCGACCAGGAAAUUGAUUCAAUCUGCAGCAUCGAGAGAGCCGAC |
| | AACGAUAAGGAGUAUCUAGUGCUUACACUUACAAAGAACGAUCUGGAUAAGGCCAAUA |
| | AGGACAAGGCAAAUAGAUACUUCAGCCCUAACUUCAAGGUUAAGCUUUACUUCACCAA |
| | GACA |
| | [TCD37 nt. seq.] |
| 72 | AUGAGCGCCGAGGGCUACCAGUACCGGGCCCUGUACGACUACAAGAAGGAGCGGGAGG |
| | AGGACAUCGACCUGCACCUGGGCGACAUCCUGACCGUGAACAAGGGCAGCCUGGUGGC |
| | CCUGGGCUUCUCCGACGGCCAGGAAGCCAGGCCCGAGGAGAUCGGCUGGCUGAACGGC |
| | UACAACGAGACUACCGGCGAGCGGGGCGACUUCCCCGGCACCUACGUGGAGUACAUCG |
| | GCCGGAAGAAGAUCAGCCCUCCCACUCCCAAGCCCCGGCCUCCAAGACCCCUGCCCGUG |
| | GCACCUGGCAGCAGCAAGACCGAGGCCGACGUGGAACAGCAGCCCGCACCCGCCUUGCC |
| | UCCUAAGCCGCCCAAGCCCACCACCGUGGCCAACAACGGCAUGAACAACAACAUGAGCC |
| | UGCAGGACGCCGAGUGGUACUGGGGCGACAUCAGCCGGGAGGAGGUGAACGAGAAGCU |
| | GCGGGACACCGCCGACGGCACCUUCCUGGUGCGCGACGCCAGCACCAAGAUGCACGGCG |
| | ACUACACCCUGACCCUGCGGAAGGGCGGCAACAACAAGCUGAUAAAGAUCUUCCACCG |
| | GGACGGCAAGUACGGCUUCAGCGAUCCCCUGACCUUCAGCAGCGUGGUGGAGCUGAUC |
| | AACCACUACCGGAACGAGAGCCUGGCCCAGUACAACCCCAAGCUGGACGUGAAGCUGC |
| | UGUACCCCGUGAGCAAGUACCAGCAGGACCAGGUGGUGAAGGAGGACAACAUCGAGGC |
| | CGUGGGCAAGAAGCUGCACGAGUACAACACCCAGUUCCAGGAGAAGUCUCGGGAGUAC |
| | GACCGGCUGUACGAGGAGUACACCCGGACCAGCCAGGAGAUCCAGAUGAAGCGGACCG |
| | CCAUCGAGGCCUUCAACGAAACCAUCAAGAUCUUCGAGGAGCAGUGCCAGACCCAGGA |
| | GCGGUACAGCAAGGAGUACAUCGAGAAGUUCAAGCGGGAAGGCAACGAGAAGGAGAUC |
| | CAGCGGAUCAUGCACAACUACGACAAGCUGAAGUCUCGGAUCAGCGAGAUCAUCGACA |
| | GCCGGAGACGGCUGGAGGAGGAUCUGAAGAAGCAGGCCGCCGAGUACCGGGAGAUCGA |
| | CAAGCGGAUGAACAGCAUCAAGCCCGACCUGAUCCAGCUGCGGAAGACCCGGGACCAG |
| | UACCUGAUGUGGCUGACCCAGAAGGGCGUGCGGCAGAAGAAGCUGAACGAGUGGCUGG |
| | GCAACGAGAACACCGAGGACCAGUACAGCCUGGUGGAGGACGACGAGGACCUGCCCCA |
| | CCACGACGAGAAGACCUGGAACGUGGGCAGCAGCAACCGGAACAAGGCCGAGAACCUG |
| | CUGCGGGGCAAGCGGGACGGCACUUUCCUGGUGCGGGAGAGCAGCAAGCAGGGCUGCU |
| | ACGCCUGCAGCGUUGUGGUGGACGGAGAGGUGAAGCACUGCGUGAUCAACAAGACCGC |
| | CACCGGCUACGGCUUCGCCGAGCCCUACAACCUGUACAGCAGCCUGAAGGAGCUGGUG |
| | CUGCACUACCAGCACACCAGCCUGGUGCAGCACAACGACAGCCUGAACGUGACCCUGGC |
| | CUACCCCGUGUACGCCCAGCAACGGAGGGGUGGUGGAGGAUCUGGCGGCGGCGGCAGU |
| | GAUCCCGAGGAGGACACCGUGGAGAGCGUGGUGAGCCCUCCCGAGCUGCCACCCCGGA |
| | ACAUACCCCUGACCGCCAGCAGCUGCGAGGCUAAGGAGGUGCCCUUCAGCAACGAGAA |
| | UCCCCGGGCCACCGAGACGAGCCGGCCCAGCCUGAGCGAAACCCUGUUCCAGCGGCUGC |
| | AGAGCAUGGACACCAGCGGCCUGCCCGAGGAGCACCUGAAGGCCAUCCAGGACUACCU |
| | GAGCACCCAGCUGGCCCAGGACAGCGAGUUCGUGAAGACAGGCAGCAGCAGCCUGCCC |
| | CACCUGAAGAAGCUGACCACCCUGCUGUGCAAGGAGCUGUACGGCGAGGUGAUCCGGA |
| | CCCUGCCCAGCCUGGAGAGCCUGCAGCGGCUGUUCGACCAGCAGCUGUCUCCUGGACUG |
| | CGGCCUCGCCCACAGGUGCCCGGCGAGGCCAACCCCAUCAACAUGGUGAGCAAGCUGAG |
| | CCAGCUGACCAGCCUGCUGAGCAGCAUCGAGGACAAGGUGAAGGCCCUGCUGCACGAG |
| | GGCCCCGAGAGCCCACACCGGCCCUCCCUGAUACCACCCGUGACCUUCGAGGUGAAGGC |
| | CGAGAGCCUGGGCAUCCCUCAGAAGAUGCAGCUGAAGGUGGACGUGGAGAGCGGCAAG |
| | CUGAUCAUCAAGAAGUCAAAGGACGGCAGCGAGGACAAGUUCUACAGCCACAAGAAGA |
| | UCCUGCAGCUGAUCAAGAGCCAGAAGUUCCUGAACAAGCUGGUGAUCCUGGUGGAAAC |
| | CGAGAAGGAGAAGAUACUGCGGAAGGAGUACGUGUUCGCCGACAGCAAGAAGCGGGAG |
| | GGCUUCUGCCAGCUGCUGCAGCAGAUGAAGAACAAGCACAGCGAGCAGCCCGAGCCCG |
| | ACAUGAUCACCAUCUUCAUCGGCACCUGGAACAUGGGCAACGCCCCUCCACCCAAGAAG |
| | AUCACCAGCUGGUUCCUGAGCAAGGGCCAGGGCAAGACCAGGGACGACAGCGCCGACU |
| | ACAUCCCUCACGACAUCUACGUGAUCGGCACCCAGGAGGACCCGCUGAGCGAGAAGGA |
| | GUGGCUGGAGAUCCUGAAGCACAGCCUGCAGGAGAUCACCAGCGUGACCUUCAAGACC |
| | GUGGCCAUCCACACCCUGUGGAACAUCCGGAUCGUGGUGCUGGCCAAGCCCGAGCACG |
| | AGAACCGGAUCAGCCACAUCUGCACCGACAACGUGAAGACCGGCAUCGCCAACACCCUG |
| | GGCAACAAGGGCGCCGUGGGCGUGAGCUUCAUGUUCAACGGCACCAGCCUGGGCUUCG |
| | UGAACAGCCACCUGACCAGCGGCAGCGAGAAGAAGCUGCGGCGGAACCAGAACUACAU |
| | GAACAUCCUGCGGUUCCUGGCCCUGGGCGACAAGAAGCUGAGCCCCUUCAACAUCACCC |
| | ACCGGUUCACCCACCUGUUCUGGUUCGGCGACCUGAACUACCGGGUGGACCUGCCCACC |
| | UGGGAGGCCGAAACCAUCAUCCAGAAGAUCAAGCAGCAGCAGUACGCCGACCUGCUGA |
| | GCCACGACCAGCUGCUGACCGAGCGGCGGGAGCAGAAGGUGUUCCUGCACUUCGAGGA |
| | GGAGGAGAUCACCUUCGCCCCAACCUACCGGUUCGAGCGGCUGACCCGGGACAAGUAC |
| | GCCUACACCAAGCAGAAGGCCACCGGCAUGAAGUACAACCUGCCCAGCUGGUGCGACC |
| | GGGUGCUGUGGAAGUCUUACCCGCUGGUGCACGUGGUGUGCCAGAGCUACGGCAGCAC |
| | CAGCGACAUCAUGACCAGCGACCACAGCCCCGUGUUCGCCACCUUCGAGGCCGGCGUGA |
| | CCAGCCAGUUCGUGAGCAAGAACGGCCCCGGCACCGUGGACAGCCAGGGCCAGAUCGA |
| | GUUCCUGCGGUGCUACGCCACCCUGAAGACCAAGAGCCAGACCAAGUUCUACCUGGAG |
| | UUCCACAGCAGCUGCCUGGAGAGCUUCGUUAAGAGCCAGGAGGGCGAGAACGAGGAGG |
| | GCAGCGAGGGCGAGCUGGUGGUGAAGUUCGGCGAGACUCUGCCCAAGCUGAAGCCCAU |
| | CAUCAGCGACCCCGAGUACCUGCUGGACCAGCACAUCCUGAUCAGCAUCAAGAGCAGC |
| | GACAGCGACGAGAGCUACGGCGAGGGCUGCAUCGCCCUGCGGCUGGAGGCCACCGAAA |
| | CCCAGCUGCCCAUCUACACACCCCUGACCCACCACGGCGAGCUGACCGGCCACUUCCAG |
| | GGCGAGAUCAAGCUGCAGACCAGCCAGGGAAAGACCCGGGAGAAGCUGUACGACUUCG |
| | UGAAGACCGAGCGGGACGAGAGCAGCGGCCCCAAGACCCUCAAGAGCCUGACCAGCCA |
| | CGACCCCAUGAAGCAGUGGGAGGUGACCAGCCGUGCACCUCCUUGCAGCGGCAGCAGC |
| | AUCACCGAG |
| | [TCD38 nt. seq.] |
| 73 | AUGGACCCGGAAGAGGACACCGUGGAGAGCGUGGUGAGCCCUCCUGAACUCCCUCCUA |
| | GAAAUAUACCUCUGACGGCCUCCAGCUGCGAGGCAAAGGAGGUGCCCUUCUCUAACGA |
| | GAAUCCGAGAGCCACCGAGACUAGCAGACCUAGCCUGUCUGAAACCCUCUUCCAGAGG |
| | CUGCAGAGCAUGGACACCAGCGGCCUGCCUGAGGAGCAUCUUAAGGCAAUCCAGGACU |
| | ACCUGUCAACACAGCUGGCACAGGACAGCGAGUUCGUCAAGACGGGCUCAAGCUCUCU |
| | GCCUCACCUGAAGAAGCUGACAACCUUACUGUGCAAGGAACUCUACGGAGAAGUGAUC |
| | CGCACAUUGCCCAGUCUGGAGAGUCUGCAGAGACUGUUUGAUCAACAGCUGAGCCCGG |
| | GCCUGAGGCCUCGGCCUCAGGUCCCUGGCGAGGCCAACCCUAUCAACAUGGUGUCGAA |
| | GUUAUCCCAAUUAACCAGCCUAUUAUCCAGCAUAGAGGACAAGGUGAAGGCCCUGCUG |
| | CACGAGGGCCCAGAGUCCCCUCACCGCCCAAGCCUUAUCCCUCCUGUGACAUUCGAGGU |
| | UAAAGCCGAGUCCCUCGGUAUCCCUCAGAAGAUGCAGCUGAAGGUCGACGUUGAGUCA |
| | GGCAAGCUGAUUAUCAAGAAGUCUAAGGACGGCAGCGAAGACAAGUUCUACAGCCACA |
| | AGAAGAUCCUACAGCUCAUCAAGAGCCAGAAGUUUCUCAAUAAGUUAGUGAUCCUGGU |
| | CGAGACGGAGAAAGAGAAGAUCUUAAGAAAGGAGUACGUGUUCGCCGACAGCAAGAAG |
| | CGGGAGGGCUUCUGCCAGUUGCUUCAGCAAAUGAAGAACAAGCACAGCGAGCAGCCUG |
| | AACCUGACAUGAUCACAAUCUUCAUUGGCACUUGGAAUAUGGGGAACGCCCCUCCUCC |
| | UAAGAAGAUUACCAGCUGGUUUCUGAGCAAGGGCCAAGGGAAGACCAGGGACGAUAGU |
| | GCGGACUACAUCCCUCACGAUAUUUACGUGAUCGGCACCCAGGAAGACCCUCUGAGCG |
| | AGAAGGAGUGGCUGGAAAUACUGAAGCAUAGCCUGCAGGAGAUCACCUCGGUGACCUU |
| | CAAGACCGUGGCAAUACAUACCCUCUGGAACAUCCGGAUAGUUGUGCUAGCUAAGCCG |
| | GAACACGAGAACAGAAUCUCUCAUAUCUGCACCGACAACGUGAAGACCGGGAUUGCUA |
| | ACACACUGGGCAACAAGGGUGCAGUGGGAGUGAGCUUCAUGUUCAACGGCACCUCACU |
| | GGGCUUCGUGAACAGUCACCUGACAAGCGGCUCCGAGAAGAAGUUAAGACGUAACCAG |
| | AAUUAUAUGAACAUCCUGAGAUUUCUGGCUCUGGGAGACAAGAAGCUGUCUCCCUUCA |
| | ACAUAACCCAUAGAUUCACCCACCUCUUCUGGUUUGGUGACCUGAAUUACCGCGUGGA |
| | UCUACCUACCUGGGAGGCUGAGACUAUUAUACAGAAGAUUAAGCAGCAGCAGUACGCC |
| | GACCUGCUGAGCCACGACCAGCUGCUGACAGAGCGGAGAGAACAGAAGGUGUUUCUCC |
| | AUUUCGAGGAGGAAGAGAUCACAUUCGCGCCUACCUACAGGUUCGAGAGAUUGACCAG |
| | AGACAAGUACGCCUACACCAAACAGAAGGCCACCGGCAUGAAGUAUAAUUUGCCAAGC |
| | UGGUGCGACAGGGUGUUGUGGAAAUCAUACCCAUUGGUUCACGUGGUUUGCCAGUCUU |
| | ACGGCAGCACGAGCGACAUCAUGACCAGCGACCACAGCCCUGUGUUCGCCACCUUCGAG |
| | GCCGGCGUGACCAGUCAGUUCGUUUCUAAGAACGGCCCCGGCACUGUGGACAGCCAGG |
| | GGCAGAUUGAGUUCCUCAGGUGCUACGCAACCUUAAAGACCAAGAGCCAGACCAAAUU |
| | CUACCUGGAGUUCCAUAGCAGCUGCCUAGAAUCGUUCGUGAAGUCCCAAGAGGGCGAG |
| | AACGAGGAGGGCAGCGAAGGCGAGCUGGUCGUGAAAUUUGGCGAGACACUGCCUAAGC |
| | UAAAGCCUAUCAUCAGCGACCCUGAGUAUCUCCUGGACCAGCACAUACUGAUUUCAAU |
| | CAAGAGCAGCGAUUCUGACGAAAGUUACGGCGAGGGCUGCAUCGCUCUCAGACUUGAA |
| | GCUACAGAAACUCAGUUACCCAUCUACACCCCUCUGACCCACCACGGCGAGCUGACCGG |
| | CCACUUCCAGGGCGAAAUCAAACUGCAGACCUCCCAGGGCAAGACCCGGGAGAAGCUU |
| | UACGACUUCGUUAAGACAGAGAGAGACGAGUCAUCAGGCCCUAAGACCCUCAAGUCGC |
| | UUACUUCCCACGAUCCUAUGAAGCAGUGGGAAGUCACAUCCCGCGCUCCUCCCUGCAGC |
| | GGAAGCAGCAUCACAGAAAUCGGCGGAGGUGGAAGCGGCGGUGGCGGCUCUUGGUUCC |
| | ACGGCAAACUGGGAGCCGGCAGGGACGGUAGACACAUAGCCGAAAGACUGCUGACUGA |
| | AUACUGUAUCGAGACAGGCGCCCCUGACGGCUCAUUCCUGGUAAGAGAGAGUGAGACA |
| | UUUGUGGGCGACUAUACUCUGAGCUUCUGGCGCAACGGCAAAGUGCAGCACUGCAGGA |
| | UUCACUCCCGCCAGGACGCCGGGACGCCUAAGUUCUUCCUGACGGAUAACCUGGUGUU |
| | UGACUCGCUGUACGAUCUGAUCACCCACUACCAGCAGGUCCCGCUGCGGUGUAACGAG |
| | UUUGAAAUGAGACUGAGUGAGCCAGUGCCACAGACCAACGCGCACGAGUCCAAGGAGU |
| | GGUAUCACGCCAGUCUGACGAGAGCCCAGGCAGAGCACAUGCUGAUGCGUGUGCCCAG |
| | AGACGGUGCCUUUCUGGUCCGAAAGAGAAACGAGCCAAACAGCUACGCCAUCAGUUUC |
| | CGCGCCGAGGGCAAGAUCAAGCAUUGCCGCGUGCAACAGGAGGGACAGACUGUCAUGC |
| | UGGGGAAUUCCGAAUUCGACUCCCUGGUGGACCUCAUCAGCUACUACGAGAAGCACCC |
| | UCUGUACCGGAAGAUGAAACUCCGGUAUCCUAUAAACGAGGAAGCCCUCGAGAAGAUU |
| | GGUACCGCGGAACCAGAUUACGGAGCUCUGUACGAGGGCAGGAAUCCGGGCUUCUACG |
| | UGGAAGCCAAUCCAAUGCCCACAUUCAAGUGUGCCGUGAAGGCUCUAUUCGACUACAA |
| | GGCCCAGCGCGAGGACGAGUUGACGUUCAUUAAGAGCGCAAUCAUCCAGAACGUGGAG |
| | AAGCAGGAGGGCGGUUGGUGGAGGGGUGACUACGGCGGUAAGAAGCAGCUGUGGUUCC |
| | CUAGUAAUUACGUCGAGGAAAUGGUCAAC |
| | [TCD39 nt. seq.] |
| 74 | AUGUGGUUCCACGGAAAGCUAGGAGCCGGUAGGGACGGAAGACAUAUAGCCGAGAGGU |
| | UGCUGACAGAGUACUGUAUUGAAACCGGAGCCCCUGACGGCUCGUUCUUAGUCAGAGA |
| | AUCUGAGACAUUCGUGGGUGACUAUACCCUGUCGUUCUGGCGAAACGGCAAGGUGCAG |
| | CACUGUCGCAUCCACUCCAGACAGGACGCUGGAACACCUAAGUUCUUCCUUACCGACA |
| | AUCUGGUCUUCGACUCUCUUUACGAUUUGAUAACCCAUUACCAGCAGGUCCCUCUGCG |
| | CUGCAACGAGUUCGAAAUGAGACUCAGUGAACCUGUGCCUCAGACUAACGCUCACGAG |
| | AGUAAGGAGUGGUAUCACGCUUCCCUCACCCGCGCACAGGCUGAACACAUGCUCAUGA |
| | GGGUCCCACGCGACGGAGCAUUCCUGGUGAGGAAGCGUAACGAACCAAAUUCCUACGC |
| | CAUUAGCUUCCGGGCAGAGGGCAAGAUAAAGCACUGCCGAGUUCAGCAGGAGGGCCAG |
| | ACAGUGAUGCUAGGAAAUUCAGAGUUCGACUCACUUGUUGAUCUCAUUAGCUACUACG |
| | AGAAGCACCCUUUGUACAGAAAGAUGAAGCUGCGGUAUCCAAUCAACGAGGAGGCCCU |
| | GGAGAAGAUUGGCACUGCUGAACCUGACUACGGAGCCCUGUACGAGGGCCGCAAUCCG |
| | GGAUUCUACGUCGAGGCGAAUCCGAUGCCAACAUUCAAGUGCGCAGUUAAGGCCCUUU |
| | UCGAUUACAAGGCCCAGCGGGAGGACGAGCUCACUUUCAUUAAGUCUGCGAUCAUCCA |
| | GAACGUCGAGAAGCAAGAGGGAGGCUGGUGGCGCGGAGACUACGGCGGAAAGAAGCAG |
| | CUCUGGUUCCCUUCUAAUUACGUCGAGGAAAUGGUCAACGGCGGAGGAGGCUCGGGCG |
| | GCGGAGGCUCCGAUCCAGAAGAGGACACUGUAGAGUCAGUGGUCAGCCCACCGGAAUU |
| | GCCGCCUCGGAACAUUCCUUUAACAGCAUCAUCCUGUGAGGCAAAGGAGGUGCCUUUC |
| | AGCAACGAGAACCCACGCGCUACUGAGACAUCCAGACCAUCACUAUCCGAGACUCUGU |
| | UCCAAAGGCUACAGAGCAUGGAUACUUCUGGUCUGCCUGAAGAACAUCUCAAGGCAAU |
| | ACAGGAUUACCUAAGUACCCAGCUGGCUCAGGACUCCGAAUUCGUGAAGACCGGCUCU |
| | AGCUCUCUUCCGCACCUCAAGAAGCUCACGACGCUGCUGUGCAAGGAGCUCUACGGUG |
| | AAGUGAUCCGGACGCUCCCUUCCCUAGAGAGUCUACAGAGAUUGUUCGACCAGCAGCU |
| | GUCCCCUGGAUUGCGUCCACGUCCGCAAGUGCCAGGCGAGGCCAACCCUAUCAAUAUG |
| | GUGAGUAAGCUGUCACAGCUGACAAGCUUGCUAAGCAGCAUCGAGGACAAGGUGAAGG |
| | CCCUGCUCCACGAGGGUCCGGAAAGUCCACAUAGGCCUAGCCUGAUCCCACCAGUGACC |
| | UUCGAAGUCAAGGCUGAGAGUCUGGGCAUCCCACAGAAGAUGCAGCUCAAGGUCGACG |
| | UGGAAUCUGGCAAGCUGAUCAUCAAGAAGUCCAAGGACGGCUCUGAGGAUAAGUUCUA |
| | CUCUCACAAGAAGAUCUUGCAGUUAAUAAAGAGUCAGAAGUUCCUCAACAAGCUGGUG |
| | AUCUUGGUGGAAACAGAGAAGGAGAAGAUCCUGCGCAAGGAGUACGUGUUCGCCGAUU |
| | CAAAGAAGAGAGAAGGCUUCUGCCAACUGCUCCAACAGAUGAAGAAUAAGCACUCCGA |
| | ACAGCCUGAGCCUGACAUGAUUACAAUCUUCAUCGGCACCUGGAAUAUGGGUAACGCA |
| | CCACCUCCUAAGAAGAUUACCAGUUGGUUCCUCAGCAAGGGCCAGGGAAAGACACGGG |
| | ACGACUCUGCAGACUAUAUUCCACACGACAUCUACGUCAUUGGAACUCAGGAGGACCC |
| | ACUGUCGGAGAAGGAGUGGCUAGAGAUCCUCAAGCAUAGCCUUCAGGAGAUUACAUCC |
| | GUUACUUUCAAGACAGUGGCCAUCCACACAUUGUGGAAUAUUAGGAUUGUCGUCCUCG |
| | CUAAGCCAGAACACGAGAACCGAAUCAGUCACAUUUGCACCGAUAACGUGAAGACGGG |
| | CAUAGCGAACACCUUGGGAAAUAAGGGAGCCGUGGGCGUGUCCUUCAUGUUCAACGGU |
| | ACAAGUCUGGGUUUCGUGAAUUCGCACCUGACGUCCGGCAGCGAGAAGAAGCUUCGGC |
| | GGAACCAGAACUACAUGAAUAUUCUGCGAUUCCUAGCGCUGGGAGAUAAGAAGUUGAG |
| | UCCUUUCAAUAUUACACACAGAUUCACGCAUCUGUUCUGGUUCGGAGAUCUUAACUAU |
| | CGCGUCGACCUGCCUACGUGGGAAGCCGAAACUAUUAUUCAGAAGAUCAAGCAACAGC |
| | AAUACGCCGACUUACUGUCUCACGAUCAGUUGCUAACCGAAAGGCGCGAGCAGAAGGU |
| | CUUCCUCCACUUCGAAGAGGAAGAGAUUACUUUCGCCCCUACCUAUAGGUUCGAACGC |
| | CUGACACGCGACAAGUACGCAUACACGAAGCAGAAGGCUACCGGCAUGAAGUACAAUU |
| | UGCCUAGCUGGUGCGAUAGAGUGCUGUGGAAGUCUUACCCGCUCGUACACGUGGUGUG |
| | UCAGUCCUACGGAUCCACUAGUGACAUCAUGACCUCCGAUCAUUCACCGGUUUUCGCU |
| | ACUUUCGAAGCUGGCGUGACGUCCCAAUUCGUGUCCAAGAACGGACCGGGCACGGUGG |
| | AUAGCCAAGGCCAAAUCGAGUUCUUGCGCUGCUACGCCACAUUGAAGACUAAGUCGCA |
| | GACCAAGUUCUAUCUCGAAUUCCACAGUAGUUGUCUGGAAAGCUUCGUAAAGAGCCAG |
| | GAAGGAGAGAACGAAGAAGGUUCCGAGGGAGAACUGGUGGUCAAGUUCGGCGAAACAC |
| | UGCCUAAGCUCAAGCCAAUAAUCUCAGAUCCGGAAUACCUGCUGGACCAACAUAUCCU |
| | GAUCUCUAUCAAGUCCUCAGACAGCGACGAAUCAUACGGCGAGGGCUGUAUUGCCCUU |
| | AGAUUGGAAGCCACUGAAACCCAACUGCCUAUCUACACACCACUGACACAUCACGGCG |
| | AGUUAACGGGACACUUCCAGGGUGAAAUUAAGCUUCAGACCUCGCAAGGAAAGACCAG |
| | AGAGAAGCUCUACGACUUCGUCAAGACUGAGCGCGACGAAAGCAGCGGCCCAAAGACA |
| | CUGAAGUCUUUGACAAGUCACGACCCAAUGAAGCAGUGGGAAGUGACUAGCCGCGCUC |
| | CUCCUUGCUCGGGAAGUUCGAUCACUGAGAUU |
| | [TCD40 nt. seq.] |
| 75 | AUGACCGCCAUCAUCAAGGAGAUCGUGAGCAGAAACGAGAGAAGAUACCAGGAGGACG |
| | GCUUCGACCUGGACCUGACCUACAUCUACCCUAACAUCAUCGCCAUGGGCUUCCCUGCC |
| | GAGAGACUGGAGGGCGUGUACAGAAACAACAUCGACGACGUGGUGAGAUUCCUGGACA |
| | GCAAGCACAAGAACCACUACAAGAUCUACAACCUGUGCGCUGAACGCCACUACGACAC |
| | CGCCAAGUUCAACUGCAGAGUGGCCCAGUACCCUUUCGAGGACCACAACCCUCCUCAGC |
| | UGGAGCUGAUCAAGCCUUUCUGCGAGGAUCUUGACCAGUGGCUGAGCGAGGACGACAA |
| | CCACGUGGCCGCCAUCCACUGCAAGGCCGGCAAGGGCAGAACCGGCGUGAUGAUCUGC |
| | GCCUACCUGCUGCACAGAGGCAAGUUCCUGAAGGCCCAGGAGGCCCUGGACUUCUACG |
| | GCGAGGUGAGAACCAGAGACAAGAAGGGCGUGACCAUCCCUAGCCAAAGGAGAUACGU |
| | GUACUACUACUCUUAUCUGCUGAAGAACCACCUGGACUACAGACCUGUGGCCCUGCUG |
| | UUCCACAAGAUGAUGUUCGAAACCAUACCGAUGUUCAGCGGCGGCACCUGCAACCCUC |
| | AGUUCGUGGUGUGCCAGCUGAAGGUGAAGAUUUACAGCAGCAACAGCGGCCCUACCAG |
| | AAGAGAGGACAAGUUCAUGUACUUCGAGUUCCCUCAGCCUCUGCCUGUGUGCGGCGAC |
| | AUCAAGGUGGAGUUCUUCCACAAGCAGAACAAGAUGCUUAAGAAGGACAAGAUGUUCC |
| | ACUUCUGGGUGAACACCUUCUUCAUCCCUGGCCCUGAGGAAACCAGCGAGGAGGUGGA |
| | GAACGGCAGCCUGUGCGACCAGGAGAUCGACAGCAUCUGCAGCAUCGAGAGAGCCGAC |
| | AACGACAAGGAGUACCUGGUGCUGACCCUGACCAAGAACGACUUGGACAAGGCCAACA |
| | AGGACAAGGCAAAUAGAUACUUCAGCCCUAACUUCAAGGUGAAGCUGUACUUCACCAA |
| | GACCGGUGGAGGAGGAUCCGGCGGUGGCGGCAGCUGGUUCCACGGCAAGCUGGGCGCC |
| | GGCAGAGACGGCAGACACAUUGCUGAGAGACUGCUGACCGAGUACUGCAUCGAAACCG |
| | GCGCCCCUGACGGCAGCUUCCUGGUGAGAGAGAGCGAAACUUUCGUGGGCGACUACAC |
| | CCUGAGCUUCUGGAGAAACGGCAAGGUGCAGCACUGCAGAAUCCACAGCAGACAGGAC |
| | GCCGGCACCCCUAAGUUCUUCCUGACCGACAACCUGGUGUUCGACAGCCUGUACGACCU |
| | GAUCACCCACUACCAGCAGGUGCCUCUGCGGUGCAACGAGUUCGAGAUGAGACUGAGC |
| | GAGCCUGUGCCUCAGACCAACGCCCACGAGAGCAAGGAGUGGUACCACGCCAGCCUGA |
| | CCAGAGCCCAGGCCGAGCACAUGCUGAUGAGAGUGCCUAGAGACGGCGCAUUCCUCGU |
| | AAGAAAGAGAAACGAGCCUAACAGCUACGCCAUCAGCUUCAGAGCCGAGGGCAAGAUC |
| | AAGCAUUGCAGAGUGCAGCAGGAGGGCCAGACCGUGAUGCUGGGCAACAGCGAAUUCG |
| | ACUCUCUGGUGGAUCUGAUCAGCUACUACGAGAAGCACCCUCUUUACCGCAAGAUGAA |
| | GCUGAGAUACCCUAUCAACGAAGAGGCCCUUGAGAAGAUCGGCACCGCCGAGCCUGAC |
| | UACGGCGCCCUGUACGAGGGCAGAAACCCUGGCUUCUACGUGGAGGCCAACCCUAUGC |
| | CUACCUUCAAGUGCGCCGUGAAGGCCCUGUUCGACUACAAGGCCCAGAGAGAGGACGA |
| | GCUGACCUUCAUCAAGUCUGCGAUUAUCCAGAACGUGGAGAAGCAAGAGGGCGGCUGG |
| | UGGAGAGGCGACUACGGCGGCAAGAAGCAGCUGUGGUUCCCUAGCAACUACGUCGAAG |
| | AGAUGGUGAAC |
| | [TCD41 nt. seq.] |
| 76 | AUGUGGUUCCACGGCAAGCUGGGCGCCGGCAGAGACGGCAGACACAUCGCCGAGAGAC |
| | UGCUGACCGAGUACUGCAUCGAAACCGGCGCCCCUGACGGCAGCUUCCUGGUGAGAGA |
| | GAGCGAAACUUUCGUGGGCGACUACACCCUGAGCUUCUGGAGAAACGGCAAGGUGCAG |
| | CACUGCAGAAUCCACAGCAGACAGGACGCCGGCACCCCUAAGUUCUUCCUGACCGACAA |
| | CCUGGUGUUCGACAGCCUGUACGACCUGAUCACCCACUACCAGCAGGUGCCUCUGCGCU |
| | GCAACGAGUUCGAGAUGAGACUGAGCGAGCCUGUGCCUCAGACCAACGCCCACGAGAG |
| | CAAGGAGUGGUACCACGCCAGCCUGACCAGAGCCCAGGCCGAGCACAUGCUGAUGAGA |
| | GUGCCUAGAGACGGCGCUUUCCUGGUUAGAAAGAGAAACGAGCCUAACAGCUACGCCA |
| | UCAGCUUCAGAGCCGAGGGCAAGAUCAAGCACUGUAGAGUGCAGCAGGAGGGCCAGAC |
| | CGUGAUGCUGGGCAACAGCGAAUUCGAUUCCCUGGUGGACCUUAUCAGCUACUACGAG |
| | AAGCACCCUCUGUACAGAAAGAUGAAGCUGAGAUACCCUAUCAACGAGGAGGCCCUGG |
| | AGAAGAUCGGCACCGCCGAGCCUGACUACGGCGCCCUGUACGAGGGCAGAAACCCUGG |
| | CUUCUACGUGGAGGCCAACCCUAUGCCUACCUUCAAGUGCGCCGUGAAGGCCCUGUUC |
| | GACUACAAGGCCCAGAGAGAGGACGAGCUGACCUUCAUCAAGAGCGCCAUCAUCCAGA |
| | ACGUGGAGAAGCAGGAAGGCGGCUGGUGGAGAGGCGAUUACGGAGGCAAGAAGCAGCU |
| | GUGGUUCCCUAGCAAUUACGUCGAGGAGAUGGUGAACGGAGGAGGUGGCUCUGGUGGA |
| | GGCGGUAGCACAGCCAUCAUCAAGGAGAUCGUGAGCAGAAACGAGAGAAGAUACCAGG |
| | AGGACGGCUUCGACCUGGACCUGACCUACAUCUACCCUAACAUCAUCGCCAUGGGCUU |
| | CCCUGCGGAACGGCUGGAGGGCGUUUAUCGCAACAACAUCGACGACGUGGUGAGAUUC |
| | CUGGACAGCAAGCACAAGAACCACUACAAGAUCUACAACCUGUGUGCGGAGCGCCACU |
| | ACGACACCGCCAAGUUCAAUUGCCGGGUGGCCCAGUACCCUUUCGAGGACCACAACCCU |
| | CCUCAGCUGGAGCUGAUCAAGCCUUUCUGCGAGGAUCUGGACCAGUGGCUGAGCGAGG |
| | ACGACAACCACGUGGCCGCCAUCCACUGCAAGGCCGGCAAGGGCAGAACCGGCGUGAU |
| | GAUCUGCGCCUACCUGCUGCACAGAGGCAAGUUCCUGAAGGCCCAGGAGGCCUUGGAC |
| | UUCUACGGCGAGGUGAGAACCAGAGACAAGAAGGGCGUGACCAUCCCUAGCCAGCGGA |
| | GAUACGUGUACUACUACUCUUACCUUCUGAAGAACCACCUGGACUACAGACCUGUGGC |
| | CCUGCUGUUCCACAAGAUGAUGUUCGAAACCAUACCAAUGUUCUCUGGAGGCACCUGC |
| | AACCCUCAGUUCGUGGUGUGCCAGCUGAAGGUGAAGAUUUAUAGCAGCAACAGCGGCC |
| | CUACCAGAAGAGAGGACAAGUUCAUGUACUUCGAGUUCCCUCAGCCUCUGCCAGUUUG |
| | UGGCGACAUCAAGGUGGAGUUCUUCCACAAGCAGAACAAGAUGUUGAAGAAGGAUAAG |
| | AUGUUCCACUUCUGGGUGAACACCUUCUUCAUCCCUGGCCCUGAGGAAACCAGCGAGG |
| | AGGUGGAGAACGGCAGCCUGUGCGACCAGGAGAUCGACAGCAUCUGCAGCAUCGAGAG |
| | AGCCGACAACGACAAGGAGUACCUGGUGCUGACCCUGACCAAGAACGACUUGGACAAG |
| | GCCAACAAGGAUAAGGCCAAUAGAUACUUCAGCCCUAACUUCAAGGUGAAGCUGUACU |
| | UCACCAAGACG |
| | [TCD42 nt. seq.] |
| 77 | AUGACCGCCAUCAUCAAGGAGAUCGUGAGCAGAAACGAGAGAAGAUACCAGGAGGACG |
| | GCUUCGACCUGGACCUGACCUACAUCUACCCUAACAUCAUCGCCAUGGGCUUCCCUGCC |
| | GAGAGACUGGAGGGCGUGUACAGAAACAACAUCGACGACGUGGUGAGAUUCCUGGACA |
| | GCAAGCACAAGAACCACUACAAGAUCUACAACCUGUGCGCCGAGAGACACUACGACAC |
| | CGCCAAGUUCAACUGCAGAGUGGCCCAGUACCCUUUCGAGGACCACAACCCUCCUCAGC |
| | UGGAGCUGAUCAAGCCUUUCUGCGAGGACCUGGACCAGUGGCUGAGCGAGGACGACAA |
| | CCACGUGGCCGCCAUCCACUGCAAGGCCGGCAAGGGCAGAACCGGCGUGAUGAUCUGC |
| | GCCUACCUGCUGCACAGAGGCAAGUUCCUGAAGGCCCAGGAGGCCCUGGACUUCUACG |
| | GCGAGGUGAGAACCAGAGACAAGAAGGGCGUGACCAUCCCUAGCCAGAGAAGAUACGU |
| | GUACUACUACAGCUACCUGCUGAAGAACCACCUGGACUACAGACCUGUGGCCCUGCUG |
| | UUCCACAAGAUGAUGUUCGAGACAAUCCCUAUGUUCAGCGGCGGCACCUGCAACCCUC |
| | AGUUCGUGGUGUGCCAGCUGAAGGUGAAGAUCUACAGCAGCAACAGCGGCCCUACCAG |
| | AAGAGAGGACAAGUUCAUGUACUUCGAGUUCCCUCAGCCUCUGCCUGUGUGCGGCGAC |
| | AUCAAGGUGGAGUUCUUCCACAAGCAGAACAAGAUGCUGAAGAAGGACAAGAUGUUCC |
| | ACUUCUGGGUGAACACCUUCUUCAUCCCUGGCCCUGAGGAGACAAGCGAGGAGGUGGA |
| | GAACGGCAGCCUGUGCGACCAGGAGAUCGACAGCAUCUGCAGCAUCGAGAGAGCCGAC |
| | AACGACAAGGAGUACCUGGUGCUGACCCUGACCAAGAACGACCUGGACAAGGCCAACA |
| | AGGAUAAGGCCAACAGAUACUUCAGCCCUAACUUCAAGGUGAAGCUGUACUUCACCAA |
| | GACCGGCGGCGGCAGCUACAGACUGAAGAAGAUCAGCAAGGAGGAGAAGACCCCUGGC |
| | UGCGUGAAGAUCAAGAAGUGC |
| | [TCD43 nt. seq.] |
| 78 | AUGGACCAGAGAGAGAUCCUGCAGAAGUUCCUGGACGAGGCCCAGAGCAAGAAGAUCA |
| | CCAAGGAGGAGUUCGCCAACGAGUUCCUGAAGCUGAAGAGACAGAGCACCAAGUACAA |
| | GGCCGACAAGACCUACCCUACCACCGUGGCCGAGAAGCCUAAGAACAUCAAGAAGAAC |
| | AGAUACAAGGACAUCCUGCCUUACGACUACAGCAGAGUGGAGCUGAGCCUGAUCACCA |
| | GCGACGAGGACAGCAGCUACAUCAACGCCAACUUCAUCAAGGGCGUGUACGGCCCUAA |
| | GGCCUACAUCGCCACCCAGGGCCCUCUGAGCACCACCCUGCUGGACUUCUGGAGAAUGA |
| | UCUGGGAGUACAGCGUGCUGAUCAUCGUGAUGGCCUGCAUGGAGUACGAGAUGGGCAA |
| | GAAGAAGUGCGAGAGAUACUGGGCCGAGCCUGGCGAGAUGCAGCUGGAGUUCGGCCCU |
| | UUCAGCGUGAGCUGCGAGGCCGAGAAGAGAAAGAGCGACUACAUCAUCAGAACCCUGA |
| | AGGUGAAGUUCAACAGCGAGACAAGAACCAUCUACCAGUUCCACUACAAGAACUGGCC |
| | UGACCACGACGUGCCUAGCAGCAUCGACCCUAUCCUGGAGCUGAUCUGGGACGUGCGC |
| | UGCUACCAGGAGGACGACAGCGUGCCUAUCUGCAUCCACUGCAGCGCCGGCUGCGGCA |
| | GAACCGGCGUGAUCUGCGCCAUCGACUACACCUGGAUGCUGCUGAAGGACGGCAUCAU |
| | CCCUGAGAACUUCAGCGUGUUCAGCCUGAUCAGAGAGAUGAGAACCCAGCGACCUAGC |
| | CUGGUGCAGACCCAGGAGCAGUACGAGCUGGUGUACAACGCCGUGCUGGAGCUGUUCG |
| | GUGGCGGCGGCUCUGGCGGUGGAGGCAGCUACAGACUGAAGAAGAUCAGCAAGGAGGA |
| | GAAGACCCCUGGCUGCGUGAAGAUCAAGAAGUGC |
| | [TCD44 nt. seq.] |
| 79 | AUGGACCAGAGAGAGAUCCUGCAGAAGUUCCUGGACGAGGCCCAGAGCAAGAAGAUCA |
| | CCAAGGAGGAGUUCGCCAACGAGUUCCUGAAGCUGAAGAGACAGGCCACCAAGUACAA |
| | GGCCGACAAGACCUACCCUACCACCGUGGCCGAGAAGCCUAAGAACAUCAAGAAGAAC |
| | AGAUACAAGGACAUCCUGCCUUACGACUACAGCAGAGUGGAGCUGAGCCUGAUCACCA |
| | GCGACGAGGACAGCAGCUACAUCAACGCCAACUUCAUCAAGGGCGUGUACGGCCCUAA |
| | GGCCUACAUCGCCACCCAGGGCCCUCUGAGCACCACCCUGCUGGACUUCUGGAGAAUGA |
| | UCUGGGAGUACAGCGUGCUGAUCAUCGUGAUGGCCUGCAUGGAGUACGAGAUGGGCAA |
| | GAAGAAGUGCGAGAGAUACUGGGCCGAGCCUGGCGAGAUGCAGCUGGAGUUCGGCCCU |
| | UUCAGCGUGAGCUGCGAGGCCGAGAAGAGAAAGAGCGACUACAUCAUCAGAACCCUGA |
| | AGGUGAAGUUCAACAGCGAAACAAGAACCAUCUACCAGUUCCACUACAAGAACUGGCC |
| | UGACCACGACGUGCCUAGCAGCAUCGACCCUAUCCUGGAGCUGAUCUGGGACGUGCGU |
| | UGCUACCAGGAGGACGACAGCGUGCCUAUCUGCAUCCACUGCAGCGCCGGCUGCGGCA |
| | GAACCGGCGUGAUCUGCGCCAUCGACUACACCUGGAUGCUGCUGAAGGACGGCAUCAU |
| | CCCUGAGAACUUCAGCGUGUUCAGCCUGAUCAGAGAGAUGAGAACCCAACGGCCUAGC |
| | CUGGUGCAGACCCAGGAGCAGUACGAGCUGGUGUACAACGCCGUGCUGGAGCUGUUCG |
| | GCGGUGGUGGCUCUGGCGGCGGAGGCUCCUACAGACUGAAGAAGAUCAGCAAGGAGGA |
| | GAAGACCCCUGGCUGCGUGAAGAUCAAGAAGUGC |
| | [TCD45 nt. seq.] |
| 80 | AUGGGCUGCGGCUGCAGCAGCCACCCUGAGGACGACUGGAUGGAGAACAUCGACGUGU |
| | GCGAGAACUGCCACUACCCUAUCGUGCCUCUGGACGGCAAGGGCACCCUGCUGAUCAG |
| | AAACGGCAGCGAGGUGCGGGAUCCUCUGGUGACCUACGAGGGCAGCAACCCUCCUGCC |
| | AGCCCUCUGCAGGACAACCUGGUGAUCGCCCUGCACAGCUACGGCGGUGGAGGCUCUG |
| | GUGGAGGUGGUUCUUUCGCCAACGAGUUCCUGAAGCUGAAGAGACAGGCCACCAAGUA |
| | CAAGGCCGACAAGACCUACCCUACCACCGUGGCCGAGAAGCCUAAGAACAUCAAGAAG |
| | AACAGAUACAAGGACAUCCUGCCUUACGACUACAGCAGAGUGGAGCUGAGCCUGAUCA |
| | CCAGCGACGAGGACAGCAGCUACAUCAACGCCAACUUCAUCAAGGGCGUGUACGGCCC |
| | UAAGGCCUACAUCGCCACCCAGGGCCCUCUGAGCACCACCCUGCUGGACUUCUGGAGAA |
| | UGAUCUGGGAGUACAGCGUGCUGAUCAUCGUGAUGGCCUGCAUGGAGUACGAGAUGGG |
| | CAAGAAGAAGUGCGAGAGAUACUGGGCCGAGCCUGGCGAGAUGCAGCUGGAGUUCGGC |
| | CCUUUCAGCGUGAGCUGCGAGGCCGAGAAGAGAAAGAGCGACUACAUCAUCAGAACCC |
| | UGAAGGUGAAGUUCAACAGCGAAACGAGAACCAUCUACCAGUUCCACUACAAGAACUG |
| | GCCUGACCACGACGUGCCUAGCAGCAUCGACCCUAUCCUGGAGCUGAUCUGGGACGUG |
| | AGGUGCUACCAGGAGGACGACAGCGUGCCUAUCUGCAUCCACUGCAGCGCCGGCUGCG |
| | GCAGAACCGGCGUGAUCUGCGCCAUCGACUACACCUGGAUGCUGCUGAAGGACGGCAU |
| | CAUCCCUGAGAACUUCAGCGUGUUCAGCCUGAUCAGAGAGAUGAGAACCCAGCGCCCU |
| | AGCCUGGUGCAGACCCAGGAGCAGUACGAGCUGGUGUACAACGCCGUGCUGGAGCUGG |
| | GAGGCGGUGGCUCUGGUAAGCCUAGCACC |
| | [TCD46 nt. seq.] |
| 81 | MPDPAAHLPFFYGSISRAEALEHLKLAGMADGLFLLRQCLRSLGGYVLSLVHDVRFHHFPIER |
| | QLNGTYAIAGGKAHCGPAELCEFYSRDPDGLPCNLRKPCNRPSGLEPQPGVFDCLRDAMVRD |
| | YVRQTWKLEGEALEQAIISQAPQVEKLIATTAHERMPWYHSSLTREEAERKLYSGAQTDGKFL |
| | LRPRKEQGTYALSLIYGKTVYHYLISQDKAGKYCIPEGTKFDTLWQLVEYLKLKADGLIYCLK |
| | EACPNSSASNASGAAAPTLPAHPSTLTHPQRRIDTLNSDGYTPEPARITSPDKPRPMPMDTSVY |
| | ESPYSDPEELKDKKLFLKRDNLFWEEFESLQKQEVKNLHQRLEGQRPENKGKNRYKNILPFDH |
| | SRVILQGRDSNIPGSDYINANYIKNQLLGPDENAKTYIASQGCLEATVNDFWQMAWQENSRVI |
| | VMTTREVEKGRNKCVPYWPEVGMQRAYGPYSVTNCGEHDTTEYKLRTLQVSPLDNGDLIREI |
| | WHYQYLSWPDHGVPSEPGGVLSFLDQINQRQESLPHAGPIIVHCSAGIGRTGTIIVIDMLMENIS |
| | TKGLDCDIDIQKTIQMVRAQRSGMVQTEAQYKFIYVAIAQF |
| | [TCD1 a.a. seq.] |
| 82 | MPDPAAHLPFFYGSISRAEALEHLKLAGMADGLFLLRQCLRSLGGYVLSLVHDVRFHHFPIER |
| | QLNGTYAIAGGKAHCGPAELCEFYSRDPDGLPCNLRKPCNRPSGLEPQPGVFDCLRDAMVRD |
| | YVRQTWKLEGEALEQAIISQAPQVEKLIATTAHERMPWYHSSLTREEAERKLYSGAQTDGKFL |
| | LRPRKEQGTYALSLIYGKTVYHYLISQDKAGKYCIPEGTKFDTLWQLVEYLKLKADGLIYCLK |
| | EACPNSSASNASGAAAPTLPAHPSTLTHPQRRIDTLNSDGATPEPARITSPDKPRPMPMDTSVA |
| | ESPASDPEELKDKKLFLKRDNLFWEEFESLQKQEVKNLHQRLEGQRPENKGKNRYKNILPFDH |
| | SRVILQGRDSNIPGSDYINANYIKNQLLGPDENAKTYIASQGCLEATVNDFWQMAWQENSRVI |
| | VMTTREVEKGRNKCVPYWPEVGMQRAYGPYSVTNCGEHDTTEYKLRTLQVSPLDNGDLIREI |
| | WHYQYLSWPDHGVPSEPGGVLSFLDQINQRQESLPHAGPIIVHCSAGIGRTGTIIVIDMLMENIS |
| | TKGLDCDIDIQKTIQMVRAQRSGMVQTEAQYKFIYVAIAQF |
| | [TCD2 a.a. seq.] |
| 83 | MPDPAAHLPFFYGSISRAEALEHLKLAGMADGLFLLRQCLRSLGGYVLSLVHDVRFHHFPIER |
| | QLNGTYAIAGGKAHCGPAELCEFYSRDPDGLPCNLRKPCNRPSGLEPQPGVFDCLRDAMVRD |
| | YVRQTWKLEGEALEQAIISQAPQVEKLIATTAHERMPWYHSSLTREEAERKLYSGAQTDGKFL |
| | LRPRKEQGTYALSLIYGKTVYHYLISQDKAGKYCIPEGTKFDTLWQLVEYLKLKADGLIYCLK |
| | EACGGGGSGGGGSFWEEFESLQKQEVKNLHQRLEGQRPENKGKNRYKNILPFDHSRVILQGR |
| | DSNIPGSDYINANYIKNQLLGPDENAKTYIASQGCLEATVNDFWQMAWQENSRVIVMTTREV |
| | EKGRNKCVPYWPEVGMQRAYGPYSVTNCGEHDTTEYKLRTLQVSPLDNGDLIREIWHYQYL |
| | SWPDHGVPSEPGGVLSFLDQINQRQESLPHAGPIIVHCSAGIGRTGTIIVIDMLMENISTKGLDC |
| | DIDIQKTIQMVRAQRSGMVQTEAQYKFIYVAIAQF |
| | [TCD3 a.a. seq.] |
| 84 | MPDPAAHLPFFYGSISRAEALEHLKLAGMADGLFLLRQCLRSLGGYVLSLVHDVRFHHFPIER |
| | QLNGTYAIAGGKAHCGPAELCEFYSRDPDGLPCNLRKPCGGGGSGGGGSGGGGSGGGGSWY |
| | HSSLTREEAERKLYSGAQTDGKFLLRPRKEQGTYALSLIYGKTVYHYLISQDKAGKYCIPEGT |
| | KFDTLWQLVEYLKLKADGLIYCLKEACGGGGSGGGGSFWEEFESLQKQEVKNLHQRLEGQR |
| | PENKGKNRYKNILPFDHSRVILQGRDSNIPGSDYINANYIKNQLLGPDENAKTYIASQGCLEAT |
| | VNDFWQMAWQENSRVIVMTTREVEKGRNKCVPYWPEVGMQRAYGPYSVTNCGEHDTTEY |
| | KLRTLQVSPLDNGDLIREIWHYQYLSWPDHGVPSEPGGVLSFLDQINQRQESLPHAGPIIVHCS |
| | AGIGRTGTIIVIDMLMENISTKGLDCDIDIQKTIQMVRAQRSGMVQTEAQYKFIYVAIAQF |
| | [TCD4 a.a. seq.] |
| 85 | WFFGKIPRAKAEEMLSKQRHDGAFLIRESESAPGDFSLSVKFGNDVQHFKVLRDGAGKYFLW |
| | VVKFNSLNELVDYHRSTSVSRNQQIFLRDIEGGGGSGGGGSGGGGSFWEEFESLQKQEVKNL |
| | HQRLEGQRPENKGKNRYKNILPFDHSRVILQGRDSNIPGSDYINANYIKNQLLGPDENAKTYIA |
| | SQGCLEATVNDFWQMAWQENSRVIVMTTREVEKGRNKCVPYWPEVGMQRAYGPYSVTNC |
| | GEHDTTEYKLRTLQVSPLDNGDLIREIWHYQYLSWPDHGVPSEPGGVLSFLDQINQRQESLPH |
| | AGPIIVHCSAGIGRTGTIIVIDMLMENISTKGLDCDIDIQKTIQMVRAQRSGMVQTEAQYKFIYV |
| | AIAQF |
| | [TCD5 a.a. seq.] |
| 86 | MWYSGRISRQLAEEILMKRNHLGAFLIRESESSPGEFSVSVNYGDQVQHFKVLREASGKYFLW |
| | EEKFNSLNELVDFYRTTTIAKKRQIFLRDEEPLGGGGSGGGGSGGGGSFWEEFESLQKQEVKN |
| | LHQRLEGQRPENKGKNRYKNILPFDHSRVILQGRDSNIPGSDYINANYIKNQLLGPDENAKTYI |
| | ASQGCLEATVNDFWQMAWQENSRVIVMTTREVEKGRNKCVPYWPEVGMQRAYGPYSVTN |
| | CGEHDTTEYKLRTLQVSPLDNGDLIREIWHYQYLSWPDHGVPSEPGGVLSFLDQINQRQESLP |
| | HAGPIIVHCSAGIGRTGTIIVIDMLMENISTKGLDCDIDIQKTIQMVRAQRSGMVQTEAQYKFIY |
| | VAIAQF |
| | [TCD6 a.a. seq.] |
| 87 | MGCGCSSHPEDDWMENIDVCENCHYPIVPLDGKGTLLIRNGSEVRDPLVTYEGSNPPASPLQD |
| | NLVIALHSYEPSHDGDLGFEKGEQLRILEQSGEWWKAQSLTTGQEGFIPFNFVAKANSLEPEP |
| | WFFKNLSRKDAERQLLAPGNTHGSFLIRESESTAGSFSLSVRDFDQNQGEVVKHYKIRNLDNG |
| | GFYISPRITFPGLHELVRHYTNASDGLCTRLSRPCQTQKPQKPWWEDEWEVPRETGGGGSGG |
| | GGSGGGGSFWEEFESLQKQEVKNLHQRLEGQRPENKGKNRYKNILPFDHSRVILQGRDSNIPG |
| | SDYINANYIKNQLLGPDENAKTYIASQGCLEATVNDFWQMAWQENSRVIVMTTREVEKGRN |
| | KCVPYWPEVGMQRAYGPYSVTNCGEHDTTEYKLRTLQVSPLDNGDLIREIWHYQYLSWPDH |
| | GVPSEPGGVLSFLDQINQRQESLPHAGPIIVHCSAGIGRTGTIIVIDMLMENISTKGLDCDIDIQK |
| | TIQMVRAQRSGMVQTEAQYKFIYVAIAQF |
| | [TCD7 a.a. seq.] |
| 88 | MEEAILVPCVLGLLLLPILAMLMALCVHCHRLPGSYDSTSSDSLYPRGIQFKRPHTVAPWPPA |
| | YPPVTSYPPLSQPDLLPIPRSPQPLGGSHRTPSSRRDSDGANSVASYENEGASGIRGAQAGWGV |
| | WGPSWTRLTPVSLPPEPACEDADEDEDDYHNPGVTYAQLLPDSTPATSTAAPSAPALSTPGIR |
| | DSAFSMESIDDVTYAQLPESGESAEASLDGSREVTYAQLSQELHPGAAKTEPAALSSQEALEV |
| | EEEGAPDYENLQELN |
| | [TCD8 a.a. seq.] |
| 89 | MEEAILVPCVLGLLLLPILAMLMALCVHCHRLPGSYDSTSSDSLYPRGIQFKRPHTVAPWPPA |
| | YPPVTSYPPLSQPDLLPIPRSPQPLGGSHRTPSSRRDSDGANSVASYENEGASGIRGAQAGWGV |
| | WGPSWTRLTPVSLPPEPACEDADEDEDDYHNPGITYAAVLPDSTPATSTAAPSAPALSTPGIRD |
| | SAFSMESIDDITYAAVPESGESALASLDGSREITYAAVSQELHPGAAKTEPAALSSQEALEVEE |
| | EGAPDYENLQELN |
| | [TCD9 a.a. seq.] |
| 90 | MEEAILVPCVLGLLLLPILAMLMALCVHCHRLPGSYDSTSSDSLYPRGIQFKRPHTVAPWPPA |
| | YPPVTSYPPLSQPDLLPIPRSPQPLGGSHRTPSSRRDSDGANSVASYENEGASGIRGAQAGWGV |
| | WGPSWTRLTPVSLPPEPACEDADEDEDDYHNPGALVVLPDSTPATSTAAPSAPALSTPGIRDS |
| | AFSMESIDDAVNVPESGESAEASLDGSREAVNVSQELHPGAAKTEPAALSSQEALEVELEGAP |
| | DYENLQELNHRQNQIKQGPPRSKDEEQKPQQRPDLAVDVLERTADKATVNGLPEKDRETDTS |
| | ALAAGSSQEVTYAQLDHWALTQRTARAVSPQSTKPMAESITYAAVARH |
| | [TCD10 a.a. seq.] |
| 91 | MEEAILVPCVLGLLLLPILAMLMALCVHCHRLPGSYDSTSSDSLYPRGIQFKRPHTVAPWPPA |
| | YPPVTSYPPLSQPDLLPIPRSPQPLGGSHRTPSSRRDSDGANSVASYENEGASGIRGAQAGWGV |
| | WGPSWTRLTPVSLPPEPACEDADEDEDDYHNPGALVVLPDSTPATSTAAPSAPALSTPGIRDS |
| | AFSMESIDDAVNVPESGESAEASLDGSREAVNVSQELHPGAAKTEPAALSSQEALEVELEGAP |
| | DYENLQELNGGGGSGGGGSFWEEFESLQKQEVKNLHQRLEGQRPENKGKNRYKNILPFDHSR |
| | VILQGRDSNIPGSDYINANYIKNQLLGPDENAKTYIASQGCLEATVNDFWQMAWQENSRVIV |
| | MTTREVEKGRNKCVPYWPEVGMQRAYGPYSVTNCGEHDTTEYKLRTLQVSPLDNGDLIREI |
| | WHYQYLSWPDHGVPSEPGGVLSFLDQINQRQESLPHAGPIIVHCSAGIGRTGTIIVIDMLMENIS |
| | TKGLDCDIDIQKTIQMVRAQRSGMVQTEAQYKFIYVAIAQF |
| | [TCD11 a.a. seq.] |
| 92 | MEEAILVPCVLGLLLLPILAMLMALCVHCHRLPGSYDSGFGGGGSGGGGSGGGGSFWEEFES |
| | LQKQEVKNLHQRLEGQRPENKGKNRYKNILPFDHSRVILQGRDSNIPGSDYINANYIKNQLLG |
| | PDENAKTYIASQGCLEATVNDFWQMAWQENSRVIVMTTREVEKGRNKCVPYWPEVGMQRA |
| | YGPYSVTNCGEHDTTEYKLRTLQVSPLDNGDLIREIWHYQYLSWPDHGVPSEPGGVLSFLDQI |
| | NQRQESLPHAGPIIVHCSAGIGRTGTIIVIDMLMENISTKGLDCDIDIQKTIQMVRAQRSGMVQT |
| | EAQYKFIYVAIAQF |
| | [TCD12 a.a. seq.] |
| 93 | MEEAILVPCVLGLLLLPILAMLMALCVHCHRLPGSYDSHRQNQIKQGPPRSKDEEQKPQQRPD |
| | LAVDVLERTADKATVNGLPEKDRETDTSALAAGSSQEVTYAQLDHWALTQRTARAVSPQST |
| | KPMAESITYAAVARH |
| | [TCD13 a.a. seq.] |
| 94 | MEEAILVPCVLGLLLLPILAMLMALCVHCHRLPMSAIQAAWPSGTECIAKYNFHGTAEQDLPF |
| | CKGDVLTIVAVTKDPNAYKAKNKVGREGIIPANYVQKREGVKAGTKLSLMPWFHGKITREQA |
| | ERLLYPPETGLFLVKESTNYPGDYTLCVSCDGKVEHYRIMYHASKLSIDEEVYFENLMQLVAH |
| | YTSDADGLCTRLIKPKVMEGTVAAQDEFYRSGWALNMKELKLLQTIGKGEFGDVMLGDYRG |
| | NKVAVKCIKNDATAQAFLAEASVMTQLRHSNLVQLLGVIVEEKGGLYIVTEYMAKGSLVDY |
| | LRSRGRSVLGGDCLLKFSLDVCEAMEYLEGNNFVHRDLAARNVLVSEDNVAKVSDFGLTKE |
| | ASSTQDTGKLPVKWTAPEALREKKFSTKSDVWSFGILLWEIYSFGRVPYPRIPLKDVVPRVEK |
| | GYKMDAPDGCPPAVYEVMKNCWHLDAAMRPSFLQLREQLEHIKTHELHL |
| | [TCD14 a.a. seq.] |
| 95 | MGPAGSLLGSGQMQITLWGSLAAVAIFFVITFLIFLCSSCDREKKPRMSAIQAAWPSGTECIAK |
| | YNFHGTAEQDLPFCKGDVLTIVAVTKDPNAYKAKNKVGREGIIPANYVQKREGVKAGTKLSL |
| | MPWFHGKITREQAERLLYPPETGLFLVKESTNYPGDYTLCVSCDGKVEHYRIMYHASKLSIDE |
| | EVYFENLMQLVAHYTSDADGLCTRLIKPKVMEGTVAAQDEFYRSGWALNMKELKLLQTIGK |
| | GEFGDVMLGDYRGNKVAVKCIKNDATAQAFLAEASVMTQLRHSNLVQLLGVIVEEKGGLYI |
| | VTEYMAKGSLVDYLRSRGRSVLGGDCLLKFSLDVCEAMEYLEGNNFVHRDLAARNVLVSED |
| | NVAKVSDFGLTKEASSTQDTGKLPVKWTAPEALREKKFSTKSDVWSFGILLWEIYSFGRVPYP |
| | RIPLKDVVPRVEKGYKMDAPDGCPPAVYEVMKNCWHLDAAMRPSFLQLREQLEHIKTHELH |
| | L |
| | [TCD15 a.a. seq.] |
| 96 | MGCGCSSHPEDDWMENIDVCENCHYPIVPLDGKGTLLIRNGSEVRDPLVTMSAIQAAWPSGT |
| | ECIAKYNFHGTAEQDLPFCKGDVLTIVAVTKDPNAYKAKNKVGREGIIPANYVQKREGVKAG |
| | TKLSLMPWFHGKITREQAERLLYPPETGLFLVKESTNYPGDYTLCVSCDGKVEHYRIMYHAS |
| | KLSIDEEVYFENLMQLVAHYTSDADGLCTRLIKPKVMEGTVAAQDEFYRSGWALNMKELKL |
| | LQTIGKGEFGDVMLGDYRGNKVAVKCIKNDATAQAFLAEASVMTQLRHSNLVQLLGVIVEE |
| | KGGLYIVTEYMAKGSLVDYLRSRGRSVLGGDCLLKFSLDVCEAMEYLEGNNFVHRDLAARN |
| | VLVSEDNVAKVSDFGLTKEASSTQDTGKLPVKWTAPEALREKKFSTKSDVWSFGILLWEIYSF |
| | GRVPYPRIPLKDVVPRVEKGYKMDAPDGCPPAVYEVMKNCWHLDAAMRPSFLQLREQLEHI |
| | KTHELHL |
| | [TCD16 a.a. seq.] |
| 97 | MGCVQCKDKEATKLTEERDGSLNQSSGYRYGTDPTPQHYPSFGVTSIPNYMSAIQAAWPSGT |
| | ECIAKYNFHGTAEQDLPFCKGDVLTIVAVTKDPNAYKAKNKVGREGIIPANYVQKREGVKAG |
| | TKLSLMPWFHGKITREQAERLLYPPETGLFLVKESTNYPGDYTLCVSCDGKVEHYRIMYHAS |
| | KLSIDEEVYFENLMQLVAHYTSDADGLCTRLIKPKVMEGTVAAQDEFYRSGWALNMKELKL |
| | LQTIGKGEFGDVMLGDYRGNKVAVKCIKNDATAQAFLAEASVMTQLRHSNLVQLLGVIVEE |
| | KGGLYIVTEYMAKGSLVDYLRSRGRSVLGGDCLLKFSLDVCEAMEYLEGNNFVHRDLAARN |
| | VLVSEDNVAKVSDFGLTKEASSTQDTGKLPVKWTAPEALREKKFSTKSDVWSFGILLWEIYSF |
| | GRVPYPRIPLKDVVPRVEKGYKMDAPDGCPPAVYEVMKNCWHLDAAMRPSFLQLREQLEHI |
| | KTHELHL |
| | [TCD17 a.a. seq.] |
| 98 | MGSNKSKPKDMSAIQAAWPSGTECIAKYNFHGTAEQDLPFCKGDVLTIVAVTKDPNAYKAK |
| | NKVGREGIIPANYVQKREGVKAGTKLSLMPWFHGKITREQAERLLYPPETGLFLVKESTNYPG |
| | DYTLCVSCDGKVEHYRIMYHASKLSIDEEVYFENLMQLVAHYTSDADGLCTRLIKPKVMEGT |
| | VAAQDEFYRSGWALNMKELKLLQTIGKGEFGDVMLGDYRGNKVAVKCIKNDATAQAFLAE |
| | ASVMTQLRHSNLVQLLGVIVEEKGGLYIVTEYMAKGSLVDYLRSRGRSVLGGDCLLKFSLDV |
| | CEAMEYLEGNNFVHRDLAARNVLVSEDNVAKVSDFGLTKEASSTQDTGKLPVKWTAPEALR |
| | EKKFSTKSDVWSFGILLWEIYSFGRVPYPRIPLKDVVPRVEKGYKMDAPDGCPPAVYLVMKN |
| | CWHLDAAMRPSFLQLREQLEHIKTHELHL |
| | [TCD18 a.a. seq.] |
| 99 | MELAILVPCVLGLLLLPILAMLMALCVHCHRLPLKLLQTIGKGEFGDVMLGDYRGNKVAVKC |
| | IKNDATAQAFLAEASVMTQLRHSNLVQLLGVIVEEKGGLYIVTEYMAKGSLVDYLRSRGRSV |
| | LGGDCLLKFSLDVCEAMEYLEGNNFVHRDLAARNVLVSEDNVAKVSDFGLTKEASSTQDTG |
| | KLPVKWTAPEALREKKFSTKSDVWSFGILLWEIYSFGRVPYPRIPLKDVVPRVEKGYKMDAPD |
| | GCPPAVYEVMKNCWHLDAAMRPSFLQLREQLEHIKTHELH |
| | [TCD19 a.a. seq.] |
| 100 | MGPAGSLLGSGQMQITLWGSLAAVAIFFVITFLIFLCSSCDREKKPRLKLLQTIGKGEFGDVML |
| | GDYRGNKVAVKCIKNDATAQAFLAEASVMTQLRHSNLVQLLGVIVEEKGGLYIVTEYMAKG |
| | SLVDYLRSRGRSVLGGDCLLKFSLDVCEAMEYLEGNNFVHRDLAARNVLVSEDNVAKVSDF |
| | GLTKEASSTQDTGKLPVKWTAPEALREKKFSTKSDVWSFGILLWEIYSFGRVPYPRIPLKDVVP |
| | RVEKGYKMDAPDGCPPAVYLVMKNCWHLDAAMRPSFLQLREQLEHIKTHELH |
| | [TCD20 a.a. seq.] |
| 101 | MGCGCSSHPEDDWMENIDVCENCHYPIVPLDGKGTLLIRNGSEVRDPLVTLKLLQTIGKGEFG |
| | DVMLGDYRGNKVAVKCIKNDATAQAFLAEASVMTQLRHSNLVQLLGVIVEEKGGLYIVTEY |
| | MAKGSLVDYLRSRGRSVLGGDCLLKFSLDVCEAMEYLEGNNFVHRDLAARNVLVSEDNVAK |
| | VSDFGLTKEASSTQDTGKLPVKWTAPEALREKKFSTKSDVWSFGILLWEIYSFGRVPYPRIPLK |
| | DVVPRVEKGYKMDAPDGCPPAVYEVMKNCWHLDAAMRPSFLQLREQLEHIKTHELH |
| | [TCD21 a.a. seq.] |
| 102 | MGCVQCKDKEATKLTEERDGSLNQSSGYRYGTDPTPQHYPSFGVTSIPNYLKLLQTIGKGEFG |
| | DVMLGDYRGNKVAVKCIKNDATAQAFLAEASVMTQLRHSNLVQLLGVIVEEKGGLYIVTEY |
| | MAKGSLVDYLRSRGRSVLGGDCLLKFSLDVCEAMEYLEGNNFVHRDLAARNVLVSEDNVAK |
| | VSDFGLTKEASSTQDTGKLPVKWTAPEALREKKFSTKSDVWSFGILLWEIYSFGRVPYPRIPLK |
| | DVVPRVEKGYKMDAPDGCPPAVYEVMKNCWHLDAAMRPSFLQLREQLEHIKTHELH |
| | [TCD22 a.a. seq.] |
| 103 | MGSNKSKPKDLKLLQTIGKGEFGDVMLGDYRGNKVAVKCIKNDATAQAFLAEASVMTQLRH |
| | SNLVQLLGVIVEEKGGLYIVTEYMAKGSLVDYLRSRGRSVLGGDCLLKFSLDVCEAMEYLEG |
| | NNFVHRDLAARNVLVSEDNVAKVSDFGLTKEASSTQDTGKLPVKWTAPEALREKKFSTKSD |
| | VWSPGILLWEIYSFGRVPYPRIPLKDVVPRVEKGYKMDAPDGCPPAVYLVMKNICWHLDAAM |
| | RPSFLQLREQLEHIKTHELH |
| | [TCD23 a.a. seq.] |
| 104 | MELAILVPCVLGLLLLPILAMLMALCVHCHRLPFWEEFESLQKQEVKNLHQRLEGQRPENKG |
| | KNRYKNILPFDHSRVILQGRDSNIPGSDYINANYIKNQLLGPDENAKTYIASQGCLEATVNDFW |
| | QMAWQENSRVIVMTTREVEKGRNKCVPYWPEVGMQRAYGPYSVTNCGEHDTTEYKLRTLQ |
| | VSPLDNGDLIREIWHYQYLSWPDHGVPSEPGGVLSFLDQINQRQESLPHAGPIIVHCSAGIGRT |
| | GTIIVIDMLMENISTKGLDCDIDIQKTIQMVRAQRSGMVQTEAQYKFIYVAIAQF |
| | [TCD24 a.a. seq.] |
| 105 | MGPAGSLLGSGQMQITLWGSLAAVAIFFVITFLIFLCSSCDREKKPRFWEEPESLQKQEVKNLH |
| | QRLEGQRPENKGKNRYKNILPFDHSRVILQGRDSNIPGSDYINANYIKNQLLGPDENAKTYIAS |
| | QGCLEATVNDFWQMAWQENSRVIVMTTREVEKGRNKCVPYWPEVGMQRAYGPYSVTNCG |
| | EHDTTEYKLRTLQVSPLDNGDLIREIWHYQYLSWPDHGVPSEPGGVLSFLDQINQRQESLPHA |
| | GPIIVHCSAGIGRTGTIIVIDMLMENISTKGLDCDIDIQKTIQMVRAQRSGMVQTEAQYKFIYVA |
| | IAQF |
| | [TCD25 a.a. seq.] |
| 106 | MGCGCSSHPEDDWMENIDVCENCHYPIVPLDGKGTLLIRNGSEVRDPLVTFWEEFESLQKQE |
| | VKNLHQRLEGQRPENKGKNRYKNILPFDHSRVILQGRDSNIPGSDYINANYIKNQLLGPDENA |
| | KTYIASQGCLEATVNDFWQMAWQENSRVIVMTTREVEKGRNKCVPYWPEVGMQRAYGPYS |
| | VTNCGEHDTTEYKLRTLQVSPLDNGDLIREIWHYQYLSWPDHGVPSEPGGVLSFLDQINQRQE |
| | SLPHAGPIIVHCSAGIGRTGTIIVIDMLMENISTKGLDCDIDIQKTIQMVRAQRSGMVQTEAQYK |
| | FIYVAIAQF |
| | [TCD26 a.a. seq.] |
| 107 | MGCVQCKDKEATKLTEERDGSLNQSSGYRYGTDPTPQHYPSFGVTSIPNYFWEEFESLQKQE |
| | VKNLHQRLEGQRPENKGKNRYKNILPFDHSRVILQGRDSNIPGSDYINANYIKNQLLGPDENA |
| | KTYIASQGCLEATVNDFWQMAWQENSRVIVMTTREVEKGRNKCVPYWPEVGMQRAYGPYS |
| | VTNCGEHDTTEYKLRTLQVSPLDNGDLIREIWHYQYLSWPDHGVPSEPGGVLSFLDQINQRQE |
| | SLPHAGPIIVHCSAGIGRTGTIIVIDMLMENISTKGLDCDIDIQKTIQMVRAQRSGMVQTEAQYK |
| | FIYVAIAQF |
| | [TCD27 a.a. seq.] |
| 108 | MGSNKSKPKDFWEEFESLQKQEVKNLHQRLEGQRPENKGKNRYKNILPFDHSRVILQGRDSNI |
| | PGSDYINANYIKNQLLGPDENAKTYIASQGCLEATVNDFWQMAWQENSRVIVMTTREVEKGR |
| | NKCVPYWPEVGMQRAYGPYSVTNCGEHDTTEYKLRTLQVSPLDNGDLIREIWHYQYLSWPD |
| | HGVPSEPGGVLSFLDQINQRQESLPHAGPIIVHCSAGIGRTGTIIVIDMLMENISTKGLDCDIDIQ |
| | KTIQMVRAQRSGMVQTEAQYKFIYVAIAQF |
| | [TCD28 a.a. seq.] |
| 109 | MEEAILVPCVLGLLLLPILAMLMALCVHCHRLPVRWFHRDLSGLDAETLLKGRGVHGSFLAR |
| | PSRKNQGDFSLSVRVGDQVTHIRIQNSGDFYDLYGGEKFATLTELVEYYTQQQGVLQDRDGTI |
| | IHLKYPLNCSDPTSERWYHGHMSGGQAETLLQAKGEPWTFLVRESLSQPGDFVLSVLSDQPK |
| | AGPGSPLRVTHIKVMCEGGRYTVGGLETFDSLTDLVEHFKKTGIEEASGAFVYLRQPYYATRV |
| | NAADIENRVLELNKKQESEDTAKAGFWEEFESLQKQEVKNLHQRLEGQRPENKGKNRYKNIL |
| | PFDHSRVILQGRDSNIPGSDYINANYIKNQLLGPDENAKTYIASQGCLEATVNDFWQMAWQE |
| | NSRVIVMTTREVEKGRNKCVPYWPEVGMQRAYGPYSVTNCGEHDTTEYKLRTLQVSPLDNG |
| | DLIREIWHYQYLSWPDHGVPSEPGGVLSFLDQINQRQESLPHAGPIIVHCSAGIGRTGTIIVIDM |
| | LMENISTKGLDCDIDIQKTIQMVRAQRSGMVQTEAQYKFIYVAIAQF |
| | [TCD29 a.a. seq.] |
| 110 | MGPAGSLLGSGQMQITLWGSLAAVAIFFVITFLIFLCSSCDREKKPRVRWFHRDLSGLDAETLL |
| | KGRGVHGSFLARPSRKNQGDFSLSVRVGDQVTHIRIQNSGDFYDLYGGEKFATLTELVEYYT |
| | QQQGVLQDRDGTIIHLKYPLNCSDPTSERWYHGHMSGGQAETLLQAKGEPWTFLVRESLSQP |
| | GDFVLSVLSDQPKAGPGSPLRVTHIKVMCEGGRYTVGGLETFDSLTDLVEHFKKTGILEASGA |
| | FVYLRQPYYATRVNAADIENRVLELNKKQESEDTAKAGFWEEFESLQKQEVKNILHQRLEGQR |
| | PENKGKNRYKNILPFDHSRVILQGRDSNIPGSDYINANYIKNQLLGPDENAKTYIASQGCLEAT |
| | VNDFWQMAWQENSRVIVMTTREVEKGRNKCVPYWPEVGMQRAYGPYSVTNCGEHDTTEY |
| | KLRTLQVSPLDNGDLIREIWHYQYLSWPDHGVPSEPGGVLSFLDQINQRQESLPHAGPIIVHCS |
| | AGIGRTGTIIVIDMLMENISTKGLDCDIDIQKTIQMVRAQRSGMVQTEAQYKFIYVAIAQF |
| | [TCD30 a.a. seq.] |
| 111 | MGCGCSSHPEDDWMENIDVCENCHYPIVPLDGKGTLLIRNGSEVRDPLVTVRWFHRDLSGLD |
| | AETLLKGRGVHGSFLARPSRKNQGDFSLSVRVGDQVTHIRIQNSGDFYDLYGGEKFATLTELV |
| | EYYTQQQGVLQDRDGTIIHLKYPLNCSDPTSERWYHGHMSGGQAETLLQAKGEPWTFLVRES |
| | LSQPGDFVLSVLSDQPKAGPGSPLRVTHIKVMCEGGRYTVGGLETFDSLTDLVEHFKKTGIEE |
| | ASGAFVYLRQPYYATRVNAADIENRVLELNKKQESEDTAKAGFWEEFESLQKQEVKNLHQRL |
| | EGQRPENKGKNRYKNILPFDHSRVILQGRDSNIPGSDYINANYIKNQLLGPDENAKTYIASQGC |
| | LEATVNDFWQMAWQENSRVIVMTTREVEKGRNKCVPYWPEVGMQRAYGPYSVTNCGEHDT |
| | TEYKLRTLQVSPLDNGDLIREIWHYQYLSWPDHGVPSEPGGVLSFLDQINQRQESLPHAGPIIV |
| | HCSAGIGRTGTIIVIDMLMENISTKGLDCDIDIQKTIQMVRAQRSGMVQTEAQYKFIYVAIAQF |
| | [TCD31 a.a. seq.] |
| 112 | MGCVQCKDKEATKLTEERDGSLNQSSGYRYGTDPTPQHYPSFGVTSIPNYVRWFHRDLSGLD |
| | AETLLKGRGVHGSFLARPSRKNQGDFSLSVRVGDQVTHIRIQNSGDFYDLYGGEKFATLTELV |
| | EYYTQQQGVLQDRDGTIIHLKYPLNCSDPTSERWYHGHMSGGQAETLLQAKGEPWTFLVRES |
| | LSQPGDFVLSVLSDQPKAGPGSPLRVTHIKVMCEGGRYTVGGLETFDSLTDLVEHFKKTGIEE |
| | ASGAFVYLRQPYYATRVNAADIENRVLELNKKQESEDTAKAGFWEEFESLQKQEVKNLHQRL |
| | EGQRPENKGKNRYKNILPFDHSRVILQGRDSNIPGSDYINANYIKNQLLGPDENAKTYIASQGC |
| | LEATVNDFWQMAWQENSRVIVMTTREVEKGRNKCVPYWPEVGMQRAYGPYSVTNCGEHDT |
| | TEYKLRTLQVSPLDNGDLIREIWHYQYLSWPDHGVPSEPGGVLSFLDQINQRQESLPHAGPIIV |
| | HCSAGIGRTGTIIVIDMLMENISTKGLDCDIDIQKTIQMVRAQRSGMVQTEAQYKFIYVAIAQF |
| | [TCD32 a.a. seq.] |
| 113 | MGSNKSKPKDVRWFHRDLSGLDAETLLKGRGVHGSFLARPSRKNQGDFSLSVRVGDQVTHIR |
| | IQNSGDFYDLYGGEKFATLTELVEYYTQQQGVLQDRDGTIIHLKYPLNCSDPTSERWYHGHM |
| | SGGQAETLLQAKGEPWTFLVRESLSQPGDFVLSVLSDQPKAGPGSPLRVTHIKVMCEGGRYT |
| | VGGLETFDSLTDLVEHFKKTGIEEASGAFVYLRQPYYATRVNAADIENRVLELNKKQESEDTA |
| | KAGFWEEFESLQKQEVKNLHQRLEGQRPENKGKNRYKNILPFDHSRVILQGRDSNIPGSDYIN |
| | ANYIKNQLLGPDENAKTYIASQGCLEATVNDFWQMAWQENSRVIVMTTREVEKGRNKCVPY |
| | WPEVGMQRAYGPYSVTNCGEHDTTEYKLRTLQVSPLDNGDLIREIWHYQYLSWPDHGVPSEP |
| | GGVLSFLDQINQRQESLPHAGPIIVHCSAGIGRTGTIIVIDMLMENISTKGLDCDIDIQKTIQMVR |
| | AQRSGMVQTEAQYKFIYVAIAQF |
| | [TCD33 a.a. seq.] |
| 114 | MEADALSPVGLGLLLLPFLVTLLAALCVRCRELPVSYDAVSLSKMLKKRSPLTTGVYVKMPP |
| | TEPECEKQFQPYFIPIN |
| | [TCD34 a.a. seq.] |
| 115 | MEEAILVPCVLGLLLLPILAMLMALCVHCHRLPGSYDSGFGGGGSTAVSLSKMLKKRSPLTTG |
| | VYVKMPPTEPECEKQFQPYFIPIN |
| | [TCD35 a.a. seq.] |
| 116 | MEEAILVPCVLGLLLLPILAMLMALCVHCHRLPGSYDSGFGGGGSMEKEFEQIDKSGSWAAIY |
| | QDIRHEASDFPCRVAKLPKNKNRNRYRDVSPFDHSRIKLHQEDNDYINASLIKMEEAQRSYILT |
| | QGPLPNTCGHFWEMVWEQKSRGVVMLNRVMEKGSLKCAQYWPQKEEKEMIFEDTNLKLTLI |
| | SEDIKSYYTVRQLELENLTTQETREILHFHYTTWPDFGVPESPASFLNFLFKVRESGSLSPEHGP |
| | VVVHCSAGIGRSGTFCLADTCLLLMDKRKDPSSVDIKKVLLEMRKFRMGLIQTADQLRFSYL |
| | AVIEGGKPST |
| | [TCD36 a.a. seq.] |
| 117 | MSAEGYQYRALYDYKKEREEDIDLHLGDILTVNKGSLVALGFSDGQEARPEEIGWLNGYNET |
| | TGERGDFPGTYVEYIGRKKISPPTPKPRPPRPLPVAPGSSKTEADVEQQPAPALPPKPPKPTTVA |
| | NNGMNNNMSLQDAEWYWGDISREEVNEKLRDTADGTFLVRDASTKMHGDYTLTLRKGGNN |
| | KLIKIFHRDGKYGFSDPLTFSSVVELINHYRNESLAQYNPKLDVKLLYPVSKYQQDQVVKEDN |
| | IEAVGKKLHEYNTQFQEKSREYDRLYEEYTRTSQEIQMKRTAIEAFNETIKIFEEQCQTQERYS |
| | KEYIEKFKREGNEKEIQRIMHNYDKLKSRISEIIDSRRRLEEDLKKQAAEYREIDKRMNSIKPDL |
| | IQLRKTRDQYLMWLTQKGVRQKKLNEWLGNENTEDQYSLVEDDEDLPHHDEKTWNVGSSN |
| | RNKAENLLRGKRDGTFLVRESSKQGCYACSVVVDGEVKHCVINKTATGYGFAEPYNLYSSLK |
| | ELVLHYQHTSLVQHNDSLNVTLAYPVYAQQRRGGGGSGGGGSTAIIKEIVSRNERRYQEDGF |
| | DLDLTYIYPNIIAMGFPAERLEGVYRNNIDDVVRFLDSKHKNHYKIYNLCAERHYDTAKFNCR |
| | VAQYPFEDHNPPQLELIKPFCEDLDQWLSEDDNHVAAIHCKAGKGRTGVMICAYLLHRGKFL |
| | KAQEALDFYGEVRTRDKKGVTIPSQRRYVYYYSYLLKNHLDYRPVALLFHKMMFETIPMFSG |
| | GTCNPQFVVCQLKVKIYSSNSGPTRREDKFMYFEFPQPLPVCGDIKVEFFHKQNKMLKKDKM |
| | FHFWVNTFFIPGPEETSEEVENGSLCDQEIDSICSIERADNDKEYLVLTLTKNIDLDKANKDKAN |
| | RYFSPNFKVKLYFTKT |
| | [TCD37 a.a. seq.] |
| 118 | MSAEGYQYRALYDYKKEREEDIDLHLGDILTVNKGSLVALGFSDGQEARPEEIGWLNGYNET |
| | TGERGDFPGTYVEYIGRKKISPPTPKPRPPRPLPVAPGSSKTEADVEQQPAPALPPKPPKPTTVA |
| | NNGMNNNMSLQDAEWYWGDISREEVNEKLRDTADGTFLVRDASTKMHGDYTLTLRKGGNN |
| | KLIKIFHRDGKYGFSDPLTFSSVVELINHYRNESLAQYNPKLDVKLLYPVSKYQQDQVVKEDN |
| | IEAVGKKLHEYNTQFQEKSREYDRLYEEYTRTSQEIQMKRTAIEAFNETIKIFEEQCQTQERYS |
| | KEYIEKFKREGNEKEIQRIMHNYDKLKSRISEIIDSRRRLEEDLKKQAAEYREIDKRMNSIKPDL |
| | IQLRKTRDQYLMWLTQKGVRQKKLNEWLGNENTEDQYSLVEDDEDLPHHDEKTWNVGSSN |
| | RNKAENLLRGKRDGTFLVRESSKQGCYACSVVVDGEVKHCVINKTATGYGFAEPYNLYSSLK |
| | ELVLHYQHTSLVQHNDSLNVTLAYPVYAQQRRGGGGSGGGGSDPEEDTVESVVSPPELPPRNI |
| | PLTASSCEAKEVPFSNENPRATETSRPSLSETLFQRLQSMDTSGLPEEHLKAIQDYLSTQLAQDS |
| | EFVKTGSSSLPHLKKLTTLLCKELYGEVIRTLPSLESLQRLFDQQLSPGLRPRPQVPGEANPINM |
| | VSKLSQLTSLLSSIEDKVKALLHEGPESPHRPSLIPPVTFEVKAESLGIPQKMQLKVDVESGKLII |
| | KKSKDGSEDKFYSHKKILQLIKSQKFLNKLVILVETEKEKILRKEYVFADSKKREGFCQLLQQ |
| | MKNKHSEQPEPDMITIFIGTWNMGNAPPPKKITSWFLSKGQGKTRDDSADYIPHDIYVIGTQED |
| | PLSEKEWLEILKHSLQEITSVTFKTVAIHTLWNIRIVVLAKPEHENRISHICTDNVKTGIANTLGN |
| | KGAVGVSFMFNGTSLGFVNSHLTSGSEKKLRRNQNYMNILRFLALGDKKLSPFNITHRFTHLF |
| | WFGDLNYRVDLPTWEALTIIQKIKQQQYADLLSHDQLLTERREQKVFLHFEEEEITFAPTYRFE |
| | RLTRDKYAYTKQKATGMKYNLPSWCDRVLWKSYPLVHVVCQSYGSTSDIMTSDHSPVFATF |
| | EAGVTSQFVSKNGPGTVDSQGQIEFLRCYATLKTKSQTKFYLEFHSSCLESFVKSQEGENEEGS |
| | EGELVVKFGETLPKLKPIISDPEYLLDQHILISIKSSDSDESYGEGCIALRLEATETQLPIYTPLTH |
| | HGELTGHFQGEIKLQTSQGKTREKLYDFVKTERDESSGPKTLKSLTSHDPMKQWEVTSRAPPC |
| | SGSSITE |
| | [TCD38 a.a. seq.] |
| 119 | MDPEEDTVESVVSPPELPPRNIPLTASSCEAKEVPFSNENPRATETSRPSLSETLFQRLQSMDTS |
| | GLPEEHLKAIQDYLSTQLAQDSEFVKTGSSSLPHLKKLTTLLCKELYGEVIRTLPSLESLQRLFD |
| | QQLSPGLRPRPQVPGEANPINMVSKLSQLTSLLSSIEDKVKALLHEGPESPHRPSLIPPVTFEVK |
| | AESLGIPQKMQLKVDVESGKLIIKKSKDGSEDKFYSHKKILQLIKSQKFLNKLVILVETEKEKIL |
| | RKEYVFADSKKREGFCQLLQQMKNKHSEQPEPDMITIFIGTWNMGNAPPPKKITSWFLSKGQ |
| | GKTRDDSADYIPHDIYVIGTQEDPLSEKEWLEILKHSLQEITSVTFKTVAIHTLWNIRIVVLAKP |
| | EHENRISHICTDNVKTGIANTLGNKGAVGVSFMFNGTSLGFVNSHLTSGSEKKLRRNQNYMNI |
| | LRFLALGDKKLSPFNITHRFTHLFWFGDLNYRVDLPTWEALTIIQKIKQQQYADLLSHDQLLTE |
| | RREQKVFLHFEEEEITFAPTYRFERLTRDKYAYTKQKATGMKYNLPSWCDRVLWKSYPLVHV |
| | VCQSYGSTSDIMTSDHSPVFATFEAGVTSQFVSKNGPGTVDSQGQIEFLRCYATLKTKSQTKF |
| | YLEFHSSCLESFVKSQEGENEEGSEGELVVKFGETLPKLKPIISDPEYLLDQHILISIKSSDSDESY |
| | GEGCIALRLEATETQLPIYTPLTHHGELTGHFQGLIKLQTSQGKTREKLYDFVKTERDESSGPK |
| | TLKSLTSHDPMKQWEVTSRAPPCSGSSITEIGGGGSGGGGSWFHGKLGAGRDGRHIAERLLTE |
| | YCIETGAPDGSFLVRESETFVGDYTLSFWRNGKVQHCRIHSRQDAGTPKFFLTDNLVFDSLYD |
| | LITHYQQVPLRCNEFEMRLSEPVPQTNAHESKEWYHASLTRAQAEHMLMRVPRDGAFLVRK |
| | RNEPNSYAISFRAEGKIKHCRVQQEGQTVMLGNSEFDSLVDLISYYEKHPLYRKMKLRYPINE |
| | EALEKIGTAEPDYGALYEGRNPGFYVEANPMPTFKCAVKALFDYKAQREDELTFIKSAIIQNV |
| | EKQEGGWWRGDYGGKKQLWFPSNYVEEMVN |
| | [TCD39 a.a. seq.] |
| 120 | MWFHGKLGAGRDGRHIAERLLTEYCIETGAPDGSFLVRESETFVGDYTLSFWRNGKVQHCRI |
| | HSRQDAGTPKFFLTDNLVFDSLYDLITHYQQVPLRCNEFEMRLSEPVPQTNAHESKEWYHASL |
| | TRAQAEHMLMRVPRDGAFLVRKRNEPNSYAISFRAEGKIKHCRVQQEGQTVMLGNSEFDSLV |
| | DLISYYEKHPLYRKMKLRYPINEEALEKIGTAEPDYGALYEGRNPGFYVEANPMPTFKCAVKA |
| | LFDYKAQREDELTFIKSAIIQNVEKQEGGWWRGDYGGKKQLWFPSNYVEEMVNGGGGSGGG |
| | GSDPEEDTVESVVSPPELPPRNIPLTASSCEAKEVPFSNENPRATETSRPSLSETLFQRLQSMDTS |
| | GLPEEHLKAIQDYLSTQLAQDSEFVKTGSSSLPHLKKLTTLLCKELYGEVIRTLPSLESLQRLFD |
| | QQLSPGLRPRPQVPGEANPINMVSKLSQLTSLLSSIEDKVKALLHEGPESPHRPSLIPPVTFEVK |
| | AESLGIPQKMQLKVDVESGKLIIKKSKDGSEDKFYSHKKILQLIKSQKFLNKLVILVETEKEKIL |
| | RKEYVFADSKKREGFCQLLQQMKNKHSEQPEPDMITIFIGTWNMGNAPPPKKITSWFLSKGQ |
| | GKTRDDSADYIPHDIYVIGTQEDPLSEKEWLEILKHSLQEITSVTFKTVAIHTLWNIRIVVLAKP |
| | EHENRISHICTDNVKTGIANTLGNKGAVGVSFMFNGTSLGFVNSHLTSGSEKKLRRNQNYMNI |
| | LRFLALGDKKLSPFNITHRFTHLFWFGDLNYRVDLPTWEALTIIQKIKQQQYADLLSHDQLLTE |
| | RREQKVFLHFEEEEITFAPTYRFERLTRDKYAYTKQKATGMKYNLPSWCDRVLWKSYPLVHV |
| | VCQSYGSTSDIMTSDHSPVFATFEAGVTSQFVSKNGPGTVDSQGQIEFLRCYATLKTKSQTKF |
| | YLEFHSSCLESFVKSQEGENEEGSEGELVVKFGETLPKLKPIISDPEYLLDQHILISIKSSDSDESY |
| | GEGCIALRLEATETQLPIYTPLTHHGELTGHFQGLIKLQTSQGKTREKLYDFVKTERDESSGPK |
| | TLKSLTSHDPMKQWEVTSRAPPCSGSSITEI |
| | [TCD40 a.a. seq.] |
| 121 | MTAIIKEIVSRNERRYQEDGFDLDLTYIYPNIIAMGFPAERLEGVYRNNIDDVVRFLDSKHKNH |
| | YKIYNLCAERHYDTAKFNCRVAQYPFEDHNPPQLELIKPFCEDLDQWLSEDDNHVAAIHCKA |
| | GKGRTGVMICAYLLHRGKFLKAQEALDFYGEVRTRDKKGVTIPSQRRYVYYYSYLLKNHLD |
| | YRPVALLFHKMMFETIPMFSGGTCNPQFVVCQLKVKIYSSNSGPTRREDKFMYFEFPQPLPVC |
| | GDIKVEFFHKQNKMLKKDKMFHFWVNTFFIPGPEETSEEVENGSLCDQEIDSICSIERADNDKE |
| | YLVLTLTKNDLDKANKDKANRYFSPNFKVKLYFTKTGGGGSGGGGSWFHGKLGAGRDGRHI |
| | AERLLTEYCIETGAPDGSFLVRESETFVGDYTLSPWRNGKVQHCRIHSRQDAGTPKFFLTDNL |
| | VFDSLYDLITHYQQVPLRCNEFEMRLSEPVPQTNAHESKEWYHASLTRAQAEHMLMRVPRD |
| | GAFLVRKRNEPNSYAISFRAEGKIKHCRVQQEGQTVMLGNSEFDSLVDLISYYEKHPLYRKM |
| | KLRYPINEEALEKIGTAEPDYGALYEGRNPGFYVEANPMPTFKCAVKALFDYKAQREDELTFI |
| | KSAIIQNVEKQEGGWWRGDYGGKKQLWFPSNYVEEMVN |
| | [TCD41 a.a. seq.] |
| 122 | MWFHGKLGAGRDGRHIAERLLTEYCIETGAPDGSFLVRESETFVGDYTLSFWRNGKVQHCRI |
| | HSRQDAGTPKFFLTDNLVFDSLYDLITHYQQVPLRCNEFEMRLSEPVPQTNAHESKEWYHASL |
| | TRAQAEHMLMRVPRDGAFLVRKRNEPNSYAISFRAEGKIKHCRVQQEGQTVMLGNSEFDSLV |
| | DLISYYEKHPLYRKMKLRYPINEEALEKIGTAEPDYGALYEGRNPGFYVEANPMPTFKCAVKA |
| | LFDYKAQREDELTFIKSAIIQNVEKQEGGWWRGDYGGKKQLWFPSNYVEEMVNGGGGSGGG |
| | GSTAIIKEIVSRNERRYQEDGFDLDLTYIYPNIIAMGFPAERLEGVYRNNIDDVVRFLDSKHKN |
| | HYKIYNLCAERHYDTAKFNCRVAQYPFEDHNPPQLELIKPFCEDLDQWLSEDDNHVAAIHCK |
| | AGKGRTGVMICAYLLHRGKFLKAQEALDFYGEVRTRDKKGVTIPSQRRYVYYYSYLLKNHL |
| | DYRPVALLFHKMMFETIPMFSGGTCNPQFVVCQLKVKIYSSNSGPTRREDKFMYFEFPQPLPV |
| | CGDIKVEFFHKQNKMLKKDKMFHFWVNTFFIPGPEETSEEVENGSLCDQEIDSICSIERADNDK |
| | EYLVLTLTKNDLDKANKDKANRYFSPNFKVKLYFTKT |
| | [TCD42 a.a. seq.] |
| 123 | MTAIIKEIVSRNERRYQEDGFDLDLTYIYPNIIAMGFPAERLEGVYRNNIDDVVRFLDSKHKNH |
| | YKIYNLCAERHYDTAKFNCRVAQYPFEDHNPPQLELIKPFCEDLDQWLSEDDNHVAAIHCKA |
| | GKGRTGVMICAYLLHRGKFLKAQEALDFYGEVRTRDKKGVTIPSQRRYVYYYSYLLKNHLD |
| | YRPVALLFHKMMFETIPMFSGGTCNPQFVVCQLKVKIYSSNSGPTRREDKFMYFEFPQPLPVC |
| | GDIKVEFFHKQNKMLKKDKMFHFWVNTFFIPGPEETSEEVENGSLCDQEIDSICSIERADNDKE |
| | YLVLTLTKNDLDKANKDKANRYFSPNFKVKLYFTKTGGGSYRLKKISKEEKTPGCVKIKKC |
| | [TCD43 a.a. seq.] |
| 124 | MDQREILQKFLDEAQSKKITKEEFANEFLKLKRQSTKYKADKTYPTTVAEKPKNIKKNRYKDI |
| | LPYDYSRVELSLITSDEDSSYINANFIKGVYGPKAYIATQGPLSTTLLDFWRMIWEYSVLIIVMA |
| | CMEYEMGKKKCERYWAEPGEMQLEFGPFSVSCEAEKRKSDYIIRTLKVKFNSETRTIYQFHY |
| | KNWPDHDVPSSIDPILELIWDVRCYQEDDSVPICIHCSAGCGRTGVICAIDYTWMLLKDGIIPEN |
| | FSVFSLIREMRTQRPSLVQTQEQYELVYNAVLELFGGGGSGGGGSYRLKKISKEEKTPGCVKI |
| | KKC |
| | [TCD44 a.a. seq.] |
| 125 | MDQREILQKFLDEAQSKKITKEEFANEFLKLKRQATKYKADKTYPTTVALKPKNIKKNRYKDI |
| | LPYDYSRVELSLITSDEDSSYINANFIKGVYGPKAYIATQGPLSTTLLDFWRMIWEYSVLIIVMA |
| | CMEYEMGKKKCERYWAEPGEMQLEFGPFSVSCEAEKRKSDYIIRTLKVKFNSETRTIYQFHY |
| | KNWPDHDVPSSIDPILELIWDVRCYQEDDSVPICIHCSAGCGRTGVICAIDYTWMLLKDGIIPEN |
| | FSVFSLIREMRTQRPSLVQTQEQYELVYNAVLELFGGGGSGGGGSYRLKKISKEEKTPGCVKI |
| | KKC |
| | [TCD45 a.a. seq.] |
| 126 | MGCGCSSHPEDDWMENIDVCENCHYPIVPLDGKGTLLIRNGSEVRDPLVTYEGSNPPASPLQD |
| | NLVIALHSYGGGGSGGGGSFANEFLKLKRQATKYKADKTYPTTVALKPKNIKKNRYKDILPY |
| | DYSRVELSLITSDEDSSYINANFIKGVYGPKAYIATQGPLSTTLLDFWRMIWEYSVLIIVMACM |
| | EYEMGKKKCERYWAEPGEMQLEFGPFSVSCEAEKRKSDYIIRTLKVKFNSETRTIYQFHYKN |
| | WPDHDVPSSIDPILELIWDVRCYQEDDSVPICIHCSAGCGRTGVICAIDYTWMLLKDGIIPENFS |
| | VFSLIREMRTQRPSLVQTQEQYELVYNAVLELGGGGSGKPST |
| | [TCD46 a.a. seq.] |
| 127 | MPGGPGVLQALPATIFLLFLLSAVYLGPGCQALWMHKVPASLMVSLGEDAHFQCPHNSSNNA |
| | NVTWWRVLHGNYTWPPEFLGPGEDPNGTLIIQNVNKSHGGIYVCRVQEGNESYQQSCGTYLR |
| | VRQPPPRPFLDMGEGTKNRIITAEGIILLFCAVVPGTLLLFRKRWQNEKLGLDAGDEYEDENLA |
| | EGLNLDDCSMAEDISRGLQGTYQDVGSLNIGDVQLEKP |
| | [hCD79a ITAM(Y/A)] |
| 128 | MPGGPGVLQALPATIFLLFLLSAVYLGPGCQALWMHKVPASLMVSLGEDAHFQCPHNSSNNA |
| | NVTWWRVLHGNYTWPPEFLGPGEDPNGTLIIQNVNKSHGGIYVCRVQEGNESYQQSCGTYLR |
| | VRQPPPRPFLDMGEGTKNRIITAEGIILLFCAVVPGTLLLFRKRWQNEKLGL |
| | [hCD79a(1-176)] |
| 129 | MARLALSPVPSHWMVALLLLLSAEPVPAARSEDRYRNPKGSACSRIWQSPRFIARKRGFTVK |
| | MHCYMNSASGNVSWLWKQEMDENPQQLKLEKGRMEESQNESLATLTIQGIRFEDNGIYFCQ |
| | QKCNNTSEVYQGCGTELRVMGFSTLAQLKQRNTLKDGIIMIQTLLIILFIIVPIFLLLDKDDSKA |
| | GMEEDHTAEGLDIDQTATAEDIVTLRTGEVKWSVGEHPGQE |
| | [hCD79b ITAM(Y/A)] |
| 130 | MARLALSPVPSHWMVALLLLLSAEPVPAARSEDRYRNPKGSACSRIWQSPRFIARKRGFTVK |
| | MHCYMNSASGNVSWLWKQEMDENPQQLKLEKGRMEESQNESLATLTIQGIRFEDNGIYFCQ |
| | QKCNNTSEVYQGCGTELRVMGFSTLAQLKQRNTLKDGIIMIQTLLIILFIIVPIFLLLDKD |
| | [hCD79b(1-184)] |
| 131 | MPPPRLLFFLLFLTPMEVRPEEPLVVKVEEGDNAVLQCLKGTSDGPTQQLTWSRESPLKPFLK |
| | LSLGLPGLGIHMRPLAIWLFIFNVSQQMGGFYLCQPGPPSEKAWQPGWTVNVEGSGELFRWN |
| | VSDLGGLGCGLKNRSSEGPSSPSGKLMSPKLYVWAKDRPEIWEGEPPCLPPRDSLNQSLSQDL |
| | TMAPGSTLWLSCGVPPDSVSRGPLSWTHVHPKGPKSLLSLELKDDRPARDMWVMETGLLLPR |
| | ATAQDAGKYYCHRGNLTMSFHLEITARPVLWHWLLRTGGWKVSAVTLAYLIFCLCSLVGILH |
| | L |
| | [hCD19(ecto-TM)] |
| 132 | MPPPRLLFFLLFLTPMEVRPEEPLVVKVEEGDNAVLQCLKGTSDGPTQQLTWSRESPLKPFLK |
| | LSLGLPGLGIHMRPLAIWLFIFNVSQQMGGFYLCQPGPPSEKAWQPGWTVNVEGSGELFRWN |
| | VSDLGGLGCGLKNRSSEGPSSPSGKLMSPKLYVWAKDRPEIWEGEPPCLPPRDSLNQSLSQDL |
| | TMAPGSTLWLSCGVPPDSVSRGPLSWTHVHPKGPKSLLSLELKDDRPARDMWVMETGLLLPR |
| | ATAQDAGKYYCHRGNLTMSFHLEITARPVLWHWLLRTGGWKVSAVTLAYLIFCLCSLVGILH |
| | LQRALVLRRKRKRMTDPTRRFFKVTPPPGSGPQNQAGNVLSLPTPTSGLGRAQRWAAGLGGT |
| | APSAGNPSSDVQADGALGSRSPPGVGPEEEEGEGAEEPDSEEDSEFYENDSNLGQDQLSQDGS |
| | GAENPEDEPLGPEDEDSFSNAESYENEDEELTQPVARTMDFLSPHGSAWDPSREATSLGSQSA |
| | EDMRGILYAAPQLRSIRGQPGPNHEEDADSYENMDNPDGPDPAWGGGGRMGTWSTR |
| | [hCD19(Y/A)] |
| 133 | MWFLTTLLLWVPVDGQVDTTKAVITLQPPWVSVFQEETVTLHCEVLHLPGSSSTQWFLNGTA |
| | TQTSTPSYRITSASVNDSGEYRCQRGLSGRSDPIQLEIHRGWLLLQVSSRVFTEGEPLALRCHA |
| | WKDKLVYNVLYYRNGKAFKFFHWNSNLTILKTNISHNGTYHCSGMGKHRYTSAGISVTVKE |
| | LFPAPVLNASVTSPLLEGNLVTLSCETKLLLQRPGLQLYFSFYMGSKTLRGRNTSSEYQILTAR |
| | REDSGLYWCEAATEDGNVLKRSPELELQVLGLQLPTPVWFHVLFYLAVGIMFLVNTVLWVTI |
| | [hCD64(ecto-TM)] |
| 134 | MILTSFGDDMWLLTTLLLWVPVGGEVVNATKAVITLQPPWVSIFQKENVTLWCEGPHLPGDS |
| | STQWFINGTAVQISTPSYSIPEASFQDSGEYRCQIGSSMPSDPVQLQIHNDWLLLQASRRVLTEG |
| | EPLALRCHGWKNKLVYNVVFYRNGKSFQFSSDSEVAILKTNLSHSGIYHCSGTGRHRYTSAG |
| | VSITVKELFTTPVLRASVSSPFPEGSLVTLNCETNLLLQRPGLQLHFSFYVGSKILEYRNTSSEY |
| | HIARAEREDAGFYWCEVATEDSSVLKRSPELELQVLGPQSSAPVWFHILFYLSVGIMFSLNTVL |
| | YV |
| | [mCD64(ecto-TM)] |
| 135 | MPGGLEALRALPLLLFLSYACLGPGCQALRVEGGPPSLTVNLGEEARLTCENNGRNPNITWWF |
| | SLQSNITWPPVPLGPGQGTTGQLFFPEVNKNHRGLYWCQVIENNILKRSCGTYLRVRNPVPRP |
| | FLDMGEGTKNRIITAEGIILLFCAVVPGTLLLFRKRWQNEKFGVDMPDDYEDENLAEGLNLDD |
| | CSMAEDISRGLQGTYQDVGNLHIGDAQLEKP |
| | [mCD79a ITAM(Y/A)] |
| 136 | MATLVLSSMPCHWLLFLLLLFSGEPVPAMTSSDLPLNFQGSPCSQIWQHPRFAAKKRSSMVKF |
| | HCYTNHSGALTWFRKRGSQQPQELVSEEGRIVQTQNGSVYTLTIQNIQYEDNGIYFCKQKCDS |
| | ANHNVTDSCGTELLVLGFSTLDQLKRRNTLKDGIILIQTLLIILFIIVPIFLLLDKDDGKAGMEED |
| | HTAEGLNIDQTATAEDIVTLRTGEVKWSVGEHPGQE |
| | [mCD79b ITAM(Y/A)] |
| 137 | MPPPRLLFFLLFLTPMEVRPQKSLLVEVEEGGNVVLPCLPDSSPVSSEKLAWYRGNQSTPFLEL |
| | SPGSPGLGLHVGSLGILLVIVNVSDHMGGFYLCQKRPPFKDIWQPAWTVNVEDSGEMFRWNA |
| | SDVRDLDCDLRNRSSGSHRSTSGSQLYVWAKDHPKVWGTKPVCAPRGSSLNQSLINQDLTVA |
| | PGSTLWLSCGVPPVPVAKASISWTHVHPRRPNVSLLSLSLGGEHPVREMWVWGSLLLLPQAT |
| | ALDEGTYYCLRGNLTIERHVKVIARSAVWLWLLRTGGWIVPVVTLVYVIFCMVSLVAFLYC |
| | [mCD19(ecto-TM)] |
| 138 | MPSPLPVSFLLFLTLVGGRPQKSLLVEVEEGGNVVLPCLPDSSPVSSEKLAWYRGNQSTPFLEL |
| | SPGSPGLGLHVGSLGILLVIVNVSDHMGGFYLCQKRPPFKDIWQPAWTVNVEDSGEMFRWNA |
| | SDVRDLDCDLRNRSSGSHRSTSGSQLYVWAKDHPKVWGTKPVCAPRGSSLNQSLINQDLTVA |
| | PGSTLWLSCGVPPVPVAKASISWTHVHPRRPNVSLLSLSLGGEHPVREMWVWGSLLLLPQAT |
| | ALDEGTYYCLRGNLTIERHVKVIARSAVWLWLLRTGGWIVPVVTLVYVIFCMVSLVAFLYCQ |
| | RAFILRRKRKRMTDPARRFFKVTPPSGNGTQNQYGNVLSLPTSTSGQAHAQRWAAGLGSVPG |
| | SAGNPRIQVQDTGAQSHETGLELEGEAALEPDSEEGSEFYENDSNLGQDQVSQDGSGAENPED |
| | EPMGPEEEDSFSNAESYENADEELAQPVGRMMDFLSPHGSAWDPSREASSLGSQSAEDMRGI |
| | LYAAPQLHSIQSGPSHEEDADSYENMDKSDDLEPAWEGEGHMGTWGTT |
| | [mCD19(Y/A)] |
| 139 | MPGGLEALRALPLLLFLSYACLGPGCQALRVEGGPPSLTVNLGEEARLTCENNGRNPNITWWF |
| | SLQSNITWPPVPLGPGQGTTGQLFFPEVNKNHRGLYWCQVIENNILKRSCGTYLRVRNPVPRP |
| | FLDMGEGTKNRIITAEGIILLFCAVVPGTLLLFRKRWQNEKFGV |
| | [mCD79a(ecto-TM)] |
| 140 | MATLVLSSMPCHWLLFLLLLFSGEPVPAMTSSDLPLNFQGSPCSQIWQHPRFAAKKRSSMVKF |
| | HCYTNHSGALTWFRKRGSQQPQELVSEEGRIVQTQNGSVYTLTIQNIQYEDNGIYFCKQKCDS |
| | ANHNVTDSCGTELLVLGFSTLDQLKRRNTLKDGIILIQTLLIILFIIVPIFLLLDKD |
| | [mCD79(ecto-TM)] |
| 141 | mPSPLPVSLLLFLTLVGGRPQNSLLVEVEEGDNVVLSCLRDSSPVSSEKLAWYRGNQSTPFLEL |
| | SLRSPDLGLHIGPLGILLVIVNVSDHRGGFYLCQKRPSFKDTWQPAWTVNVEDSGELFRWNAS |
| | DLGDLDCDLGNRSSGSHRSTSGSQLYVWATDHPEVWKTKPVCAPREISLNQSLINQDLTVAP |
| | GSTLWLSCGVPPVPVTKGSISWTHVHPKTLNVSLLSLSLGGEHPVREMWVWGSLLLLPQAKA |
| | SDEGTYYCLQGGLTIKMHVKVIARSAVWLWLLRTGGWIVPVVTLVYVIFCMVSMAAFLYF |
| | [rCD19(ecto-TM)] |
| 142 | MLGGLGVLRTLPLLLLFLSEACLGPGCQALMLERDPPSLTVNLGEEAVLTCKNIDGKNPNITW |
| | WFSLQSNSTWPPMPLGPGLGPMGKLIFPEVNKSHRGLYWCQVIESKEVKRSCGTYLRVRKQV |
| | PRPFLDMGEGTKNRIITAEGIILLFCAVVPGTLLLFRKRWQNEKFGV |
| | [rCD79a(ecto-TM)] |
| 143 | MATLVLSPVPCHWLMFLLLLLSGEPVPAMTKSDQPPIFQGSPCSKIWQHPRFAAKKRSSMVKF |
| | HCHTDYSGVMTWFRQKGNQRPQELFPEDGHISQTRNGSVYTLTLQNIQYEDNGIYFCQQKCN |
| | STEPDVTDGCGTELLVLGFSTLDQLKRRNTLKDGIIMIQTLLIILFIIVPIFLLLDKD |
| | [rCD79b(ecto-TM)] |
| 144 | YNPMMEDGISYTTLRFPEMNIPRTGDAESSEMQRPPPDCDDTVTYSALHKRQVGDYENVIPDF |
| | PEDEGIHYSELIQFGVGERPQAQENVDYVILKH |
| | [hCD22 ITIM] |
| 145 | FWEEFESLQKQEVKNLHQRLEGQRPENKGKNRYKNILPFDHSRVILQGRDSNIPGSDYINANYI |
| | KNIQLLGPDENAKTYIASQGCLEATVNDFWQMAWQENSRVIVMTTREVEKGRNKCVPYWPE |
| | VGMQRAYGPYSVTNCGEHDTTEYKLRTLQVSPLDNGDLIREIWHYQYLSWPDHGVPSEPGGV |
| | LSFLDQINQRQESLPHAGPIIVHCSAGIGRTGTIIVIDMLMENISTKGLDCDIDIQKTIQMVRAQR |
| | SGMVQTEAQYKFIYVAIAQF |
| | [hSHP1(PTP)] |
| 146 | VVALIYCRKKRISALPGYPECREMGETLPEKPANPTNPDEADKVGAENTITYSLLMHPDALEE |
| | PDDQNRI |
| | [hCD32b ITIM] |
| 147 | VSLVYLKKKQVPALPGNPDHREMGETLPEEVGEYRQPSGGSVPVSPGPPSGLEPTSSSPYNPPD |
| | LEEAAKTEAENTITYSLLKHPEALDEETEHDYQNHI |
| | [mCD32b ITIM] |
| 148 | YNPAMDDTVSYAILRFPESDTHNTGDAGTPATQAPPPNNSDSVTYSVIQKRPMGDYENVNPSC |
| | PEDESIHYSELVQFGAGKRPQAKEDVDYVTLKH |
| | [mCD22 ITIM] |
| 149 | YNLAMDDTVSYAVLRFPESDTHGAGGARSPATQGPPPNDDDTVTYSVLQKRNMGDYENVSP |
| | NCPEDESIHYSELVQFGAGKRPQAKEDVDYVTLKH |
| | [rCD22 ITIM] |
| 150 | AUGCCUGGCGGCCCUGGCGUGCUGCAGGCCCUGCCUGCCACCAUCUUCCUGCUGUUCUU |
| | ACUCAGCGCCGUGUACCUGGGACCAGGCUGCCAGGCCCUGUGGAUGCACAAGGUCCCA |
| | GCUAGCCUGAUGGUGAGCCUGGGCGAGGACGCCCACUUCCAGUGCCCUCACAACAGCA |
| | GCAACAACGCCAACGUGACCUGGUGGAGAGUGCUGCACGGCAACUACACCUGGCCUCC |
| | UGAGUUCCUGGGCCCAGGUGAAGAUCCUAACGGCACCCUGAUCAUCCAGAACGUGAAC |
| | AAGAGCCACGGCGGCAUCUACGUGUGCAGAGUGCAGGAGGGCAACGAGAGCUACCAGC |
| | AGAGCUGCGGCACCUACCUGAGAGUGAGACAGCCUCCUCCUAGACCUUUCCUGGACAU |
| | GGGAGAAGGCACCAAGAACAGAAUCAUCACCGCCGAGGGCAUCAUUCUCCUCUUCUGC |
| | GCCGUGGUGCCUGGUACCUUGCUACUUUUCAGAAAGCGGUGGCAGAACGAGAAGCUGG |
| | GCCUGGACGCCGGCGACGAGUACGAGGACGAGAACCUGGCUGAAGGCCUGAACCUGGA |
| | CGACUGCAGCAUGGCCGAGGACAUCAGCAGAGGCCUGCAGGGAACUUACCAGGACGUG |
| | GGCAGCCUGAACAUCGGCGACGUGCAGCUGGAGAAGCCUUACAACCCUAUGAUGGAGG |
| | ACGGCAUCAGCUACACCACCCUGAGAUUCCCGGAGAUGAACAUCCCUAGAACAGGCGA |
| | CGCCGAGAGCAGCGAGAUGCAGCGACCACCUCCUGACUGCGACGACACCGUGACCUACA |
| | GCGCCCUGCACAAGAGACAGGUGGGCGACUACGAGAACGUCAUCCCUGACUUCCCAGA |
| | GGACGAGGGAAUCCACUACAGCGAGCUGAUCCAGUUCGGCGUGGGCGAACGGCCACAG |
| | GCCCAGGAGAACGUGGACUACGUGAUUCUGAAGCAC |
| | [BCD1 n.t. seq.] |
| 151 | AUGCCUGGCGGCCCUGGCGUGCUGCAGGCCCUGCCUGCCACCAUCUUCCUGCUGUUCCU |
| | GCUGAGCGCCGUGUACCUGGGCCCUGGCUGCCAGGCCCUGUGGAUGCACAAGGUGCCU |
| | GCCAGCCUGAUGGUGAGCCUGGGCGAGGACGCCCACUUCCAGUGCCCUCACAACAGCA |
| | GCAACAACGCCAACGUGACCUGGUGGAGAGUGCUGCACGGCAACUACACCUGGCCUCC |
| | UGAGUUCUUAGGACCUGGCGAGGACCCUAACGGCACCCUGAUCAUCCAGAACGUGAAC |
| | AAGAGCCACGGCGGCAUCUACGUGUGCAGAGUGCAGGAGGGCAACGAGAGCUACCAGC |
| | AGAGCUGCGGCACCUACCUGAGAGUGAGACAGCCUCCUCCUAGACCUUUCCUGGACAU |
| | GGGCGAGGGCACCAAGAACAGAAUCAUCACCGCCGAGGGCAUCAUCCUGCUGUUCUGC |
| | GCCGUGGUGCCUGGCACCCUGCUGCUGUUCAGAAAGAGGUGGCAGAACGAGAAGCUGG |
| | GCCUGUACAACCCUAUGAUGGAGGACGGCAUCAGCUACACCACCCUGAGAUUCCCUGA |
| | GAUGAACAUCCCUAGAACCGGCGACGCCGAGAGCAGCGAGAUGCAGCGCCCUCCUCCU |
| | GACUGCGACGACACCGUGACCUACAGCGCCCUGCACAAGAGACAGGUGGGCGACUACG |
| | AGAACGUGAUCCCUGACUUCCCUGAGGACGAGGGCAUCCACUACAGCGAGCUGAUCCA |
| | GUUCGGCGUGGGCGAGCGUCCUCAGGCCCAGGAGAACGUGGACUACGUGAUCCUGAAG |
| | CAC |
| | [BCD2 n.t. seq.] |
| 152 | AUGGCCAGACUGGCCCUGAGCCCUGUGCCUAGCCACUGGAUGGUGGCCCUGCUGUUAC |
| | UCUUGAGCGCCGAGCCUGUGCCUGCCGCCAGAAGCGAGGACAGAUACAGAAACCCUAA |
| | GGGCAGCGCCUGCAGCAGAAUCUGGCAGAGCCCUAGAUUCAUCGCCAGAAAGAGAGGC |
| | UUCACCGUGAAGAUGCACUGCUACAUGAACAGCGCCAGCGGCAACGUGAGCUGGCUGU |
| | GGAAGCAGGAGAUGGACGAGAACCCUCAGCAGCUGAAGCUGGAGAAGGGCAGAAUGGA |
| | GGAGAGCCAGAACGAGAGCCUGGCCACCCUGACCAUCCAGGGCAUCAGAUUCGAGGAC |
| | AACGGCAUCUACUUCUGCCAGCAGAAGUGCAACAACACCAGCGAGGUGUACCAGGGCU |
| | GCGGCACCGAGCUGAGAGUGAUGGGCUUCAGCACCCUGGCCCAGCUGAAGCAGAGAAA |
| | CACCCUGAAGGACGGCAUCAUCAUGAUCCAGACCCUGCUGAUCAUCCUGUUCAUCAUC |
| | GUGCCUAUCUUCCUGCUGCUGGACAAGGACGACAGCAAGGCCGGCAUGGAGGAGGACC |
| | ACACCGCCGAGGGCCUGGACAUCGACCAGACCGCCACCGCCGAGGACAUCGUGACCCUG |
| | AGAACCGGCGAGGUGAAGUGGAGCGUGGGCGAGCAUCCAGGACAGGAGUACAACCCUA |
| | UGAUGGAAGACGGUAUCAGCUACACCACCCUGAGAUUCCCUGAGAUGAACAUCCCUAG |
| | AACCGGCGACGCCGAGAGCAGCGAGAUGCAAAGACCUCCUCCUGACUGCGACGACACC |
| | GUGACCUACAGCGCCCUGCACAAGAGACAGGUGGGCGACUACGAGAACGUGAUCCCUG |
| | ACUUCCCUGAGGACGAGGGCAUCCACUACAGCGAGCUGAUCCAGUUCGGCGUGGGCGA |
| | ACGUCCUCAGGCCCAGGAGAACGUGGACUACGUGAUCCUGAAGCAC |
| | [BCD3 nt. seq.] |
| 153 | AUGGCCAGACUGGCCCUGAGCCCUGUGCCUAGCCACUGGAUGGUGGCCCUGCUGCUGC |
| | UGCUGAGCGCCGAGCCUGUGCCUGCCGCCAGAAGCGAGGACAGAUACAGAAACCCUAA |
| | GGGCAGCGCCUGCAGCAGAAUCUGGCAGAGCCCUAGAUUCAUCGCCAGAAAGAGAGGC |
| | UUCACCGUGAAGAUGCACUGCUACAUGAACAGCGCCAGCGGCAACGUGAGCUGGCUGU |
| | GGAAGCAGGAGAUGGACGAGAACCCUCAGCAGCUGAAGCUGGAGAAGGGCAGAAUGGA |
| | GGAGAGCCAGAACGAGAGCCUGGCCACCCUGACCAUCCAGGGCAUCAGAUUCGAGGAC |
| | AACGGCAUCUACUUCUGCCAGCAGAAGUGCAACAACACCAGCGAGGUGUACCAGGGCU |
| | GCGGCACCGAGCUGAGAGUGAUGGGCUUCAGCACCCUGGCCCAGCUGAAGCAGAGAAA |
| | CACCCUGAAGGACGGCAUCAUCAUGAUCCAGACCCUGCUGAUCAUCCUGUUCAUCAUC |
| | GUGCCUAUCUUCCUGCUGCUGGACAAGGACUACAACCCUAUGAUGGAGGACGGCAUCA |
| | GCUACACCACCCUGAGAUUCCCUGAGAUGAACAUCCCUAGAACCGGCGACGCCGAGAG |
| | CAGCGAGAUGCAGAGACCUCCUCCUGACUGCGACGACACCGUGACCUACAGCGCCCUGC |
| | ACAAGAGACAGGUGGGCGACUACGAGAACGUGAUCCCUGACUUCCCUGAGGACGAGGG |
| | CAUCCACUACAGCGAGCUGAUCCAGUUCGGCGUGGGCGAGAGACCUCAGGCCCAGGAG |
| | AACGUGGACUACGUGAUCCUGAAGCAC |
| | [BCD4 nt. seq.] |
| 154 | AUGCCUCCUCCUAGACUGCUGUUCUUCCUGCUGUUCCUGACCCCUAUGGAGGUGAGAC |
| | CUGAGGAGCCUCUGGUGGUGAAGGUGGAGGAGGGCGACAACGCCGUGCUGCAGUGCCU |
| | GAAGGGCACCAGCGACGGCCCUACCCAGCAGCUGACCUGGAGCAGAGAGAGCCCUCUG |
| | AAGCCUUUCCUGAAGCUGAGCCUGGGCCUGCCUGGCCUGGGCAUCCACAUGCGACCUC |
| | UGGCCAUCUGGCUGUUCAUCUUCAACGUGAGCCAGCAGAUGGGCGGCUUCUACCUGUG |
| | CCAGCCUGGCCCUCCUAGCGAGAAGGCCUGGCAGCCUGGCUGGACCGUGAACGUGGAG |
| | GGCAGCGGCGAGCUGUUCCGCUGGAACGUGAGCGACCUGGGAGGACUGGGCUGUGGCC |
| | UGAAGAACAGAAGCAGCGAGGGCCCUAGCAGCCCUAGCGGCAAGCUGAUGAGCCCUAA |
| | GCUGUACGUGUGGGCCAAGGACAGACCUGAGAUCUGGGAGGGCGAGCCUCCUUGCCUG |
| | CCUCCUAGAGACAGCCUGAACCAGAGCCUGAGCCAGGACCUGACCAUGGCCCCUGGCAG |
| | CACCCUGUGGCUGAGCUGCGGCGUGCCUCCUGACAGCGUGAGCAGAGGCCCUCUGAGC |
| | UGGACCCACGUGCACCCUAAGGGCCCUAAGAGCCUGCUGAGCCUGGAGCUGAAGGACG |
| | ACAGACCUGCCAGAGACAUGUGGGUGAUGGAGACAGGCCUGCUGCUGCCUAGAGCCAC |
| | CGCCCAGGACGCCGGCAAGUACUACUGCCACAGAGGCAACCUGACCAUGAGCUUCCACC |
| | UGGAGAUCACCGCCAGACCUGUGCUGUGGCACUGGCUGCUGAGAACCGGCGGCUGGAA |
| | GGUGAGCGCCGUGACCCUGGCCUACCUGAUCUUCUGCCUGUGCAGCCUGGUGGGCAUC |
| | CUGCACCUCGGCGGAGGAGGAUCGGGCGGAGGUGGCUCUUACAACCCUAUGAUGGAGG |
| | ACGGCAUCAGCUACACCACCCUGAGAUUCCCUGAGAUGAACAUCCCUAGAACCGGCGA |
| | CGCCGAGAGCAGCGAGAUGCAACGACCUCCUCCUGACUGCGACGACACCGUGACCUACA |
| | GCGCCCUGCACAAGAGACAGGUGGGCGACUACGAGAACGUGAUCCCUGACUUCCCUGA |
| | GGACGAGGGCAUCCACUACAGCGAGCUGAUCCAGUUCGGCGUGGGCGAAAGGCCUCAG |
| | GCCCAGGAGAACGUGGACUACGUGAUCCUGAAGCAC |
| | [BCD5 nt. seq.] |
| 155 | AUGCCUCCUCCUAGACUGCUGUUCUUCCUGUUGUUCCUGACCCCUAUGGAGGUGAGAC |
| | CUGAGGAGCCUCUGGUGGUGAAGGUGGAGGAGGGCGACAACGCCGUGCUGCAGUGCCU |
| | GAAGGGCACCAGCGACGGCCCUACCCAGCAGCUGACCUGGAGCAGAGAGAGCCCUCUG |
| | AAGCCUUUCCUGAAGCUGAGCCUGGGCCUGCCUGGCCUGGGCAUCCACAUGCGACCUC |
| | UGGCCAUCUGGCUGUUCAUCUUCAACGUGAGCCAGCAGAUGGGCGGCUUCUACCUGUG |
| | CCAGCCUGGCCCUCCUAGCGAGAAGGCCUGGCAGCCAGGUUGGACCGUGAACGUGGAG |
| | GGCAGCGGCGAGCUGUUCCGGUGGAACGUAAGCGACCUGGGCGGACUCGGUUGCGGCC |
| | UGAAGAACAGAAGCAGCGAGGGCCCUAGCAGCCCUAGCGGCAAGCUGAUGAGCCCUAA |
| | GCUGUACGUGUGGGCCAAGGACAGACCUGAGAUCUGGGAAGGAGAGCCUCCUUGCCUG |
| | CCUCCUAGGGACAGCCUGAACCAGAGCCUGAGCCAGGACCUGACCAUGGCCCCUGGAUC |
| | UACCCUGUGGCUGAGCUGCGGCGUGCCUCCUGACAGCGUGAGCAGAGGACCACUUAGC |
| | UGGACCCACGUGCACCCUAAGGGACCAAAGAGCUUACUGUCUUUGGAGCUGAAGGACG |
| | ACCGCCCAGCCAGAGACAUGUGGGUGAUGGAAACAGGCCUGCUGCUGCCUAGAGCCAC |
| | CGCUCAGGACGCCGGCAAGUACUACUGCCACAGAGGCAACCUGACGAUGAGCUUCCAC |
| | CUGGAGAUCACCGCCAGACCUGUGCUGUGGCACUGGCUGCUGAGAACCGGCGGCUGGA |
| | AGGUGAGCGCCGUGACCCUGGCCUACCUGAUCUUCUGCCUGUGUAGCCUCGUCGGCAU |
| | ACUGCACCUGGGUGGCGGAGGUUCGGGCGGCGGCGGAUCAGGAGGAGGCGGCUCCUUC |
| | UGGGAGGAGUUCGAAAGUUUGCAGAAGCAAGAGGUGAAGAACCUGCACCAGAGACUGG |
| | AGGGUCAGAGGCCAGAGAACAAGGGCAAGAACAGGUACAAGAACAUCCUGCCUUUCGA |
| | CCACAGCAGAGUGAUCCUGCAGGGCCGGGACUCCAACAUCCCUGGCUCAGACUACAUC |
| | AACGCCAACUACAUAAAGAACCAGCUGCUGGGCCCUGACGAGAACGCCAAGACCUACA |
| | UCGCCAGCCAGGGCUGCCUGGAGGCCACUGUCAACGACUUCUGGCAGAUGGCCUGGCA |
| | GGAGAAUUCCCGAGUGAUCGUGAUGACAACCAGAGAGGUGGAGAAGGGCAGAAACAAG |
| | UGCGUGCCUUACUGGCCUGAGGUUGGCAUGCAGAGAGCCUACGGCCCUUACUCUGUGA |
| | CCAACUGUGGUGAGCACGACACCACCGAGUACAAGUUACGCACCCUGCAGGUCAGUCC |
| | ACUGGACAACGGCGACCUGAUCAGAGAGAUUUGGCACUACCAGUACCUGUCUUGGCCU |
| | GACCACGGAGUGCCAUCCGAGCCUGGUGGAGUUUUGUCCUUCCUGGACCAGAUCAAUC |
| | AGCGGCAGGAAUCCCUGCCACACGCCGGCCCUAUCAUCGUGCACUGCAGCGCCGGCAUC |
| | GGCAGAACCGGUACCAUCAUAGUCAUCGACAUGCUUAUGGAGAACAUCAGCACCAAGG |
| | GCCUGGACUGCGAUAUUGAUAUCCAGAAGACCAUCCAGAUGGUGAGAGCCCAGAGAAG |
| | CGGCAUGGUGCAGACCGAGGCCCAGUAUAAGUUCAUCUACGUGGCCAUCGCCCAGUUC |
| | [BCD6 nt. seq.] |
| 156 | AUGCCUCCUCCUAGACUGCUGUUCUUCUUGCUUUUCCUGACCCCUAUGGAGGUGAGAC |
| | CUGAGGAGCCUCUGGUGGUGAAGGUGGAGGAGGGCGACAACGCCGUGCUGCAGUGCCU |
| | GAAGGGCACCAGCGACGGCCCUACCCAGCAGCUGACCUGGAGCAGAGAGAGCCCUCUG |
| | AAGCCUUUCCUGAAGCUGAGCCUGGGCCUGCCUGGCCUGGGCAUCCACAUGCGGCCUC |
| | UGGCCAUCUGGCUGUUCAUCUUCAACGUGAGCCAGCAGAUGGGCGGCUUCUACCUGUG |
| | CCAGCCUGGCCCUCCUAGCGAGAAGGCCUGGCAGCCUGGCUGGACCGUGAACGUGGAG |
| | GGCAGCGGCGAGCUGUUCCGUUGGAACGUUAGUGACCUGGGCGGACUAGGCUGCGGCC |
| | UGAAGAACAGAAGCAGCGAGGGCCCUAGCAGCCCUAGCGGCAAGCUGAUGAGCCCUAA |
| | GCUGUACGUGUGGGCCAAGGACAGACCUGAGAUCUGGGAAGGCGAGCCUCCUUGCCUG |
| | CCACCUAGGGACAGCCUGAACCAGAGCCUGAGCCAGGACCUGACCAUGGCCCCGGGAUC |
| | CACCCUGUGGCUGAGCUGCGGCGUGCCUCCUGACAGCGUGAGCAGAGGCCCACUUAGC |
| | UGGACCCACGUGCACCCUAAGGGACCAAAGAGCUUAUUAUCCCUUGAGCUGAAGGACG |
| | ACAGGCCAGCCAGAGACAUGUGGGUGAUGGAAACCGGCCUGCUGCUGCCUAGAGCCAC |
| | CGCCCAGGACGCCGGCAAGUACUACUGCCACAGAGGCAAUCUGACAAUGAGCUUCCAC |
| | CUGGAGAUCACCGCCAGACCUGUGCUGUGGCACUGGCUGCUGAGAACCGGCGGCUGGA |
| | AGGUGAGCGCCGUGACCCUGGCCUACCUGAUCUUCUGCCUGUGCUCACUGGUCGGUAU |
| | CCUGCACCUGCAGCGCGCUCUGGUCCUCAGAAGAAAGCGAAAGCGGAUGACCGACCCU |
| | ACCAGAAGAUUCUUCAAGGUGACCCCACCACCUGGCUCUGGACCUCAGAACCAGGCCG |
| | GCAACGUGCUCUCACUGCCUACCCCAACCUCCGGUCUGGGUAGAGCCCAGAGGUGGGCC |
| | GCUGGUUUGGGCGGCACCGCCCCUAGUGCAGGUAAUCCGUCAAGCGACGUGCAGGCCG |
| | ACGGCGCCCUGGGCAGCAGAAGCCCUCCUGGAGUGGGCCCAGAGGAGGAAGAGGGUGA |
| | AGGCGCCGAGGAGCCAGAUUCCGAGGAGGACUCUGAGUUCUACGAGAACGACAGCAAC |
| | CUGGGUCAGGACCAGUUGAGUCAGGACGGUUCUGGCGCGGAGAAUCCGGAGGACGAGC |
| | CAUUGGGACCUGAGGACGAAGACUCGUUCAGCAACGCCGAGAGUUACGAGAACGAAGA |
| | CGAGGAGCUGACCCAGCCUGUGGCCAGAACCAUGGACUUCCUGAGCCCUCACGGCAGC |
| | GCCUGGGAUCCAAGUCGGGAGGCCACAAGCUUGGGUUCCCAGUCCGCUGAGGACAUGA |
| | GAGGAAUCCUCUACGCCGCCCCUCAGUUGCGGAGCAUCCGCGGCCAGCCGGGUCCAAAC |
| | CACGAGGAGGACGCAGACAGUUACGAGAACAUGGACAACCCUGACGGCCCGGACCCUG |
| | CUUGGGGCGGUGGUGGUAGAAUGGGUACUUGGAGUACCAGAUACAACCCUAUGAUGGA |
| | GGACGGUAUCAGCUACACCACCCUGAGAUUCCCAGAAAUGAACAUCCCUCGAACCGGA |
| | GACGCAGAAAGCUCCGAAAUGCAGCGCCCUCCUCCAGAUUGCGACGACACCGUGACCU |
| | ACAGCGCCCUGCACAAGAGACAGGUGGGCGACUACGAGAACGUGAUCCCUGACUUCCC |
| | UGAAGACGAGGGAAUCCACUACAGCGAGCUGAUCCAGUUCGGCGUGGGAGAGAGGCCU |
| | CAGGCCCAGGAGAACGUUGACUACGUGAUUCUGAAGCAC |
| | [BCD7 nt. seq.] |
| 157 | AUGUGGUUCCUGACCACCCUGCUGCUGUGGGUGCCUGUGGACGGCCAGGUGGACACCA |
| | CCAAGGCCGUGAUCACCCUGCAGCCUCCUUGGGUGAGCGUGUUCCAGGAGGAGACUGU |
| | GACCCUGCACUGCGAGGUGCUGCACCUGCCUGGCAGCAGCAGCACCCAGUGGUUCCUCA |
| | ACGGCACCGCCACCCAGACCAGCACCCCUAGCUACAGAAUCACCAGCGCCAGCGUGAAC |
| | GACAGCGGCGAGUACCGGUGCCAGAGAGGCCUGAGCGGCAGAAGCGACCCUAUCCAGC |
| | UGGAGAUCCACAGAGGCUGGCUGCUGCUGCAGGUGAGCAGCAGAGUGUUCACCGAGGG |
| | CGAGCCUCUGGCCCUGAGGUGCCACGCCUGGAAGGACAAGCUGGUGUACAACGUGCUG |
| | UACUACAGAAACGGCAAGGCCUUCAAGUUCUUCCACUGGAACAGCAACCUGACCAUCC |
| | UGAAGACCAACAUCAGCCACAACGGUACCUACCACUGCAGCGGCAUGGGCAAGCACAG |
| | AUAUACUUCUGCCGGCAUCAGCGUGACCGUGAAGGAGCUGUUCCCUGCCCCUGUGCUG |
| | AACGCAAGUGUGACCAGCCCUCUGCUGGAGGGCAACCUGGUGACCCUGAGCUGCGAGA |
| | CAAAGCUGCUCCUGCAAAGGCCUGGCCUGCAGCUGUACUUCAGCUUCUACAUGGGCAG |
| | CAAGACCCUGAGAGGCAGAAACACCAGCAGCGAGUACCAGAUCCUGACCGCCAGAAGA |
| | GAGGACAGCGGCCUGUACUGGUGCGAGGCCGCCACCGAGGACGGCAACGUCCUGAAGA |
| | GAAGUCCUGAGCUGGAGCUUCAGGUGCUGGGUCUGCAGCUGCCUACCCCUGUGUGGUU |
| | CCACGUGCUGUUCUACCUGGCCGUGGGCAUCAUGUUCCUGGUGAACACCGUCUUGUGG |
| | GUGACCAUCGUGGUGGCCCUGAUCUACUGCAGAAAGAAGAGAAUCAGCGCCCUGCCGG |
| | GCUACCCUGAGUGCAGAGAGAUGGGCGAGACUCUGCCUGAGAAGCCUGCCAACCCUAC |
| | CAACCCUGACGAGGCCGACAAGGUGGGCGCCGAGAACACCAUCACCUACAGCCUGCUG |
| | AUGCACCCUGACGCCCUGGAGGAGCCUGACGACCAGAACAGAAUC |
| | [BCD8 nt. seq.] |
| 158 | AUGAUCCUGACCAGCUUCGGCGACGACAUGUGGCUGCUGACCACCCUGCUGCUGUGGG |
| | UGCCUGUGGGAGGAGAGGUGGUGAACGCCACCAAGGCCGUGAUCACCCUGCAGCCUCC |
| | UUGGGUGAGCAUCUUCCAGAAGGAGAACGUGACCCUGUGGUGCGAGGGCCCUCACCUG |
| | CCUGGCGACAGCAGCACCCAGUGGUUCAUCAACGGCACCGCCGUGCAGAUCAGCACCCC |
| | UAGCUACAGCAUCCCUGAGGCCAGCUUCCAGGACAGCGGCGAGUACCGCUGCCAGAUC |
| | GGCAGCAGCAUGCCUAGCGACCCGGUCCAGCUGCAGAUCCACAACGACUGGCUACUGC |
| | UGCAGGCCAGCAGAAGAGUGCUGACCGAGGGCGAGCCUCUGGCCCUGCGAUGCCACGG |
| | CUGGAAGAACAAGCUGGUGUACAACGUGGUGUUCUACAGAAACGGCAAGUCCUUCCAA |
| | UUCAGCAGCGACAGCGAGGUGGCCAUCCUGAAGACCAACCUGAGCCACAGCGGCAUCU |
| | ACCACUGCAGCGGCACCGGCAGACACAGAUACACCAGCGCCGGCGUGAGCAUUACCGU |
| | GAAGGAGCUGUUCACCACCCCUGUGCUGAGAGCCUCAGUCUCUAGCCCUUUCCCGGAA |
| | GGCAGCCUGGUCACUCUGAACUGCGAGACAAACCUGCUACUGCAGCGGCCUGGCCUGC |
| | AGUUGCACUUCAGCUUCUACGUGGGCAGCAAGAUCCUGGAGUACCGAAAUACUAGCAG |
| | UGAGUACCACAUCGCCAGAGCCGAGAGAGAGGACGCCGGCUUCUACUGGUGUGAGGUU |
| | GCUACCGAGGAUUCCAGCGUGCUGAAGAGAAGCCCUGAGCUGGAGCUGCAGGUGCUGG |
| | GCCCGCAGAGCAGCGCCCCUGUGUGGUUCCACAUCCUGUUCUACCUGAGCGUGGGCAU |
| | CAUGUUCAGCCUGAACACCGUGCUGUACGUCGUAUCCUUGGUAUACCUGAAGAAGAAG |
| | CAGGUGCCUGCGCUCCCAGGCAACCCUGACCACAGAGAGAUGGGCGAAACCCUCCCUGA |
| | AGAGGUUGGUGAAUACCGACAGCCUAGCGGCGGCAGCGUCCCUGUAAGCCCUGGCCCU |
| | CCGUCUGGUCUGGAGCCUACAUCUAGUAGUCCAUACAACCCUCCUGACCUGGAGGAGG |
| | CCGCCAAGACCGAGGCCGAGAACACCAUCACCUACAGCCUGCUGAAGCAUCCAGAGGCU |
| | CUGGACGAGGAAACAGAGCACGACUACCAGAACCACAUC |
| | [BCD9 nt. seq.] |
| 159 | AUGCCUGGCGGCCUGGAGGCCCUGAGAGCCCUGCCUCUGCUGCUGUUCCUGAGCUACG |
| | CCUGCCUGGGCCCUGGCUGCCAGGCCCUGAGAGUGGAGGGCGGCCCUCCUAGCCUGACC |
| | GUGAACCUGGGCGAGGAGGCCAGACUGACCUGCGAGAACAACGGCAGAAACCCUAACA |
| | UCACCUGGUGGUUCAGCCUGCAGAGCAAUAUCACUUGGCCUCCUGUGCCUCUGGGUCC |
| | AGGCCAGGGCACCACCGGCCAGCUGUUCUUCCCUGAGGUGAACAAGAACCACAGAGGC |
| | CUGUACUGGUGCCAGGUGAUUGAGAAUAACAUCCUGAAGAGAAGCUGCGGCACCUACC |
| | UGAGAGUGAGAAACCCUGUGCCUAGACCUUUCCUGGACAUGGGCGAGGGCACCAAGAA |
| | CAGAAUCAUCACCGCCGAGGGCAUCAUCCUGCUGUUCUGCGCCGUGGUGCCUGGCACCC |
| | UACUGUUAUUCAGAAAGAGGUGGCAGAACGAGAAGUUCGGCGUGGACAUGCCUGACGA |
| | CUACGAGGACGAGAACCUGGCCGAGGGCCUGAACCUGGACGACUGCAGCAUGGCCGAG |
| | GACAUCAGCCGGGGUCUGCAGGGCACCUAUCAGGACGUGGGCAACCUGCACAUCGGCG |
| | ACGCCCAGCUGGAGAAGCCUUACAACCCUGCCAUGGACGACACCGUCAGUUACGCCAUC |
| | CUGAGAUUCCCUGAAAGCGACACCCACAACACUGGUGACGCCGGCACCCCUGCCACCCA |
| | GGCCCCUCCUCCUAACAACAGCGACAGCGUGACCUACAGCGUGAUCCAGAAGCGUCCUA |
| | UGGGCGAUUACGAGAACGUGAACCCUAGCUGCCCUGAAGACGAAAGCAUCCACUACAG |
| | CGAGCUGGUGCAGUUCGGCGCCGGCAAGCGACCUCAGGCCAAGGAGGACGUGGACUAC |
| | GUGACCCUGAAGCAC |
| | [BCD10 nt. seq.] |
| 160 | AUGGCCACCCUGGUGCUGAGCAGCAUGCCUUGCCACUGGCUGCUGUUCCUGCUACUGC |
| | UGUUCAGCGGCGAGCCUGUGCCUGCCAUGACCAGCAGCGACCUGCCUCUGAACUUCCA |
| | GGGCAGCCCUUGCAGCCAGAUCUGGCAGCACCCUAGAUUCGCCGCCAAGAAGAGAAGC |
| | AGCAUGGUGAAGUUCCACUGCUACACCAACCACAGCGGCGCCCUGACCUGGUUCAGAA |
| | AGAGAGGCAGCCAGCAGCCUCAGGAGCUGGUGAGCGAGGAGGGCAGAAUCGUGCAGAC |
| | CCAGAACGGCAGCGUGUACACCCUGACCAUCCAGAACAUCCAGUACGAGGACAACGGC |
| | AUCUACUUCUGCAAGCAGAAGUGCGACAGCGCCAACCACAACGUGACCGACAGCUGCG |
| | GCACCGAGCUGCUGGUGCUGGGCUUCAGCACCCUGGACCAGCUGAAGAGAAGAAACAC |
| | CCUGAAGGACGGCAUCAUCCUGAUCCAGACCCUGCUGAUCAUCCUGUUCAUCAUCGUG |
| | CCUAUCUUCCUACUCCUGGAUAAGGACGACGGCAAGGCCGGCAUGGAGGAGGACCACA |
| | CCGCCGAGGGCCUGAACAUCGACCAGACCGCCACGGCCGAGGACAUCGUGACCCUGAGA |
| | ACCGGCGAGGUGAAGUGGAGCGUGGGCGAACAUCCUGGCCAGGAGUACAACCCUGCCA |
| | UGGACGACACCGUGAGCUACGCCAUCCUGAGAUUCCCUGAGAGCGACACCCACAACACC |
| | GGCGACGCCGGCACCCCUGCCACCCAGGCCCCUCCUCCUAACAACAGCGACAGCGUGAC |
| | CUACAGCGUGAUCCAGAAGAGGCCUAUGGGCGACUACGAGAACGUGAACCCUAGCUGC |
| | CCUGAGGACGAGAGCAUCCACUACAGCGAGCUGGUGCAGUUCGGCGCCGGCAAGCGUC |
| | CUCAGGCCAAGGAGGACGUGGACUACGUUACACUGAAGCAC |
| | [BCD11 nt. seq.] |
| 161 | AUGCCUCCUCCUAGACUGCUGUUCUUCCUGCUGUUCCUGACCCCUAUGGAGGUGAGAC |
| | CUCAGAAGUCUCUGCUGGUGGAGGUGGAGGAGGGCGGCAACGUGGUGCUGCCUUGCCU |
| | GCCUGACAGCAGCCCUGUGAGCAGCGAGAAGCUGGCCUGGUACAGAGGCAACCAGAGC |
| | ACCCCUUUCCUGGAGCUGAGCCCUGGCAGCCCUGGCCUGGGCCUGCACGUGGGCAGCCU |
| | GGGCAUAUUACUGGUGAUCGUGAACGUGAGCGACCACAUGGGCGGCUUCUACCUGUGC |
| | CAGAAGCGUCCUCCUUUCAAGGACAUCUGGCAGCCUGCCUGGACCGUAAACGUGGAGG |
| | ACAGCGGCGAGAUGUUCCGAUGGAACGCCAGCGACGUGAGGGACCUGGACUGCGACCU |
| | GAGAAACAGAAGCAGCGGCAGCCACAGAAGCACGAGUGGAUCUCAGCUGUACGUGUGG |
| | GCCAAGGACCACCCUAAGGUGUGGGGCACCAAGCCUGUGUGCGCCCCUAGAGGCAGCA |
| | GCCUGAACCAGAGCCUGAUCAACCAGGACCUGACCGUGGCUCCGGGUAGCACCCUGUG |
| | GCUGAGCUGCGGCGUGCCUCCUGUGCCUGUGGCCAAGGCCAGCAUCAGCUGGACCCAC |
| | GUGCACCCUAGAAGACCUAACGUAAGCCUUCUGAGCCUGAGCCUGGGCGGCGAACACC |
| | CAGUGAGAGAGAUGUGGGUCUGGGGCUCCUUACUCCUGCUGCCUCAGGCCACCGCCCU |
| | GGACGAGGGCACCUACUACUGCCUGAGGGGAAACCUGACCAUCGAGAGACACGUGAAG |
| | GUGAUCGCCAGAAGCGCCGUGUGGCUGUGGCUGCUGAGAACCGGCGGCUGGAUCGUCC |
| | CGGUGGUGACCCUGGUGUACGUGAUCUUCUGCAUGGUGAGCCUGGUGGCCUUCCUGUA |
| | CUGCGGAGGAGGCGGCUCCGGAGGCGGAGGCAGCUACAACCCUGCCAUGGACGACACC |
| | GUGAGCUACGCCAUCCUGAGAUUCCCUGAGAGCGACACCCACAACACCGGCGACGCCGG |
| | CACCCCUGCCACCCAGGCCCCACCUCCAAACAACAGCGACAGCGUGACCUACAGCGUGA |
| | UUCAGAAGAGGCCUAUGGGCGACUACGAGAACGUGAACCCUAGCUGCCCUGAGGACGA |
| | GAGCAUCCACUACAGCGAGCUGGUGCAGUUCGGCGCCGGCAAGCGUCCACAGGCCAAG |
| | GAGGACGUGGACUACGUGACCCUGAAGCAC |
| | [BCD12 nt. seq.] |
| 162 | AUGCCUAGCCCUCUGCCUGUGAGCUUCCUGCUGUUCCUGACCCUGGUGGGCGGCAGAC |
| | CUCAGAAGUCGCUGCUGGUGGAGGUGGAGGAGGGCGGCAACGUGGUGCUGCCUUGCCU |
| | GCCUGACAGCAGCCCAGUCUCGAGCGAGAAGCUGGCCUGGUACAGAGGCAACCAGAGC |
| | ACCCCUUUCCUGGAGCUGAGCCCUGGAUCGCCUGGUCUAGGCCUGCACGUGGGCAGCC |
| | UGGGCAUACUCCUUGUAAUCGUGAACGUGAGCGACCACAUGGGCGGCUUCUACCUGUG |
| | CCAGAAGCGCCCUCCUUUCAAGGACAUUUGGCAACCUGCUUGGACUGUCAACGUGGAG |
| | GACAGCGGCGAGAUGUUCCGGUGGAACGCCAGCGACGUGAGGGACCUGGACUGCGACC |
| | UGAGAAACAGAAGCAGCGGCAGCCACAGAUCCACCAGUGGCUCCCAGCUGUACGUGUG |
| | GGCCAAGGACCACCCUAAGGUGUGGGGCACCAAGCCUGUGUGCGCCCCUAGAGGCAGC |
| | AGCCUGAACCAGUCCCUGAUCAACCAGGACCUGACCGUGGCUCCGGGCUCUACCCUGUG |
| | GCUGAGCUGCGGCGUGCCUCCUGUGCCUGUGGCCAAGGCCAGCAUCAGCUGGACCCAC |
| | GUGCACCCUAGAAGACCUAACGUGUCGCUUUUGAGCCUGUCACUUGGCGGCGAGCACC |
| | CUGUGAGAGAGAUGUGGGUCUGGGGAUCCCUUCUGUUGCUGCCUCAGGCCACCGCCCU |
| | GGACGAGGGCACCUACUACUGCCUGCGCGGUAACCUGACCAUCGAGAGACACGUGAAG |
| | GUGAUCGCCAGAAGCGCCGUGUGGCUUUGGCUUCUGAGAACCGGCGGCUGGAUCGUUC |
| | CAGUCGUGACCCUAGUGUACGUGAUCUUCUGCAUGGUUUCACUUGUGGCCUUCCUGUA |
| | CUGCCAGAGAGCCUUCAUCCUGAGAAGAAAGCGCAAGAGAAUGACCGACCCAGCUCGU |
| | CGAUUCUUCAAGGUGACCCCUCCUUCCGGAAACGGCACCCAGAACCAGUACGGAAACG |
| | UGCUAUCCCUGCCAACCUCCACUAGCGGACAGGCCCACGCCCAGCGAUGGGCCGCCGGC |
| | CUGGGAAGCGUGCCGGGAAGUGCCGGCAAUCCUCGCAUCCAGGUGCAGGACACCGGAG |
| | CACAGAGCCACGAGACGGGCUUAGAAGAGGAAGGCGAGGCCGCCGAGGAACCUGAUAG |
| | CGAAGAGGGUAGCGAGUUCUACGAGAACGACAGCAACCUGGGCCAGGACCAGGUGAGC |
| | CAGGACGGCAGCGGCGCCGAGAACCCUGAGGACGAGCCUAUGGGUCCUGAAGAGGAGG |
| | ACUCUUUCAGCAACGCCGAGUCUUACGAGAACGCCGACGAGGAGCUGGCCCAGCCUGU |
| | GGGCAGAAUGAUGGACUUCCUGUCUCCUCACGGCUCAGCCUGGGACCCUAGCAGAGAA |
| | GCUAGUAGUUUAGGCAGCCAAAGUGCAGAAGACAUGCGUGGUAUUCUAUACGCCGCCC |
| | CUCAGCUGCACAGCAUCCAGAGCGGCCCUAGUCACGAGGAAGACGCCGACAGUUACGA |
| | GAACAUGGACAAGAGCGACGACCUGGAACCUGCCUGGGAGGGAGAAGGCCAUAUGGGA |
| | ACCUGGGGCACUACCUACAACCCUGCCAUGGACGACACCGUGAGCUACGCCAUUCUCAG |
| | AUUCCCUGAGAGCGACACCCACAAUACAGGUGACGCCGGCACCCCUGCCACCCAGGCCC |
| | CUCCUCCUAACAACAGCGACAGCGUGACCUAUUCCGUCAUUCAGAAGCGUCCAAUGGG |
| | CGACUACGAGAACGUGAACCCUAGCUGUCCUGAGGACGAGAGCAUCCACUACAGCGAG |
| | CUGGUGCAGUUCGGCGCUGGCAAGCGUCCUCAGGCUAAGGAGGACGUGGACUACGUAA |
| | CACUGAAGCAC |
| | [BCD13 nt. seq.] |
| 163 | AUGCCUGGCGGCCUGGAGGCCCUGAGAGCCCUGCCUCUGCUGCUGUUCCUGAGCUACG |
| | CCUGCCUGGGCCCUGGCUGCCAGGCCCUGAGAGUGGAGGGCGGCCCUCCUAGCCUGACC |
| | GUGAACCUGGGCGAGGAGGCCAGACUGACCUGCGAGAACAACGGCAGAAACCCUAACA |
| | UCACCUGGUGGUUCAGCCUGCAGAGCAAUAUAACUUGGCCUCCUGUGCCUCUUGGUCC |
| | AGGACAGGGCACCACCGGCCAGCUGUUCUUCCCUGAGGUGAACAAGAACCACAGAGGC |
| | CUGUACUGGUGCCAGGUGAUCGAGAACAACAUCCUGAAGAGAAGCUGCGGCACCUACC |
| | UGAGAGUGAGAAACCCUGUGCCUAGACCUUUCCUGGACAUGGGCGAGGGCACCAAGAA |
| | CAGAAUCAUCACCGCCGAGGGCAUCAUCCUGCUGUUCUGCGCCGUGGUGCCUGGCACC |
| | UUGCUUCUGUUCAGAAAGCGAUGGCAGAACGAGAAGUUCGGCGUGUACAACCCUGCCA |
| | UGGACGACACCGUGAGCUACGCCAUCCUGAGAUUCCCUGAGAGCGACACCCACAACACC |
| | GGCGACGCCGGCACCCCUGCCACCCAGGCCCCUCCUCCUAACAACAGCGACAGCGUGAC |
| | CUACAGCGUGAUCCAGAAGAGGCCUAUGGGCGACUACGAGAACGUGAACCCUAGCUGC |
| | CCUGAGGACGAGAGCAUCCACUACAGCGAGCUGGUGCAGUUCGGCGCCGGCAAGCGCC |
| | CUCAGGCCAAGGAGGACGUGGACUACGUGACCCUGAAGCAC |
| | [BCD14 nt. seq.] |
| 164 | AUGGCCACCCUGGUGCUGAGCAGCAUGCCUUGCCACUGGCUGCUGUUCCUGCUGCUGC |
| | UGUUCAGCGGCGAGCCUGUGCCUGCCAUGACCAGCAGCGACCUGCCUCUGAACUUCCA |
| | GGGCAGCCCUUGCAGCCAGAUCUGGCAGCACCCUAGAUUCGCCGCCAAGAAGAGAAGC |
| | AGCAUGGUGAAGUUCCACUGCUACACCAACCACAGCGGCGCCCUGACCUGGUUCAGAA |
| | AGAGAGGCAGCCAGCAGCCUCAGGAGCUGGUGAGCGAGGAGGGCAGAAUCGUGCAGAC |
| | CCAGAACGGCAGCGUGUACACCCUGACCAUCCAGAACAUCCAGUACGAGGACAACGGC |
| | AUCUACUUCUGCAAGCAGAAGUGCGACAGCGCCAACCACAACGUGACCGACAGCUGCG |
| | GCACCGAGCUGCUGGUGCUGGGCUUCAGCACCCUGGACCAGCUGAAGAGAAGAAACAC |
| | CCUGAAGGACGGCAUCAUCCUGAUCCAGACCCUGCUGAUCAUCCUGUUCAUCAUCGUG |
| | CCUAUCUUCCUGCUGCUGGACAAGGACUACAACCCUGCCAUGGACGACACCGUGAGCU |
| | ACGCCAUCCUGAGAUUCCCUGAGAGCGACACCCACAACACCGGCGACGCCGGCACCCCU |
| | GCCACCCAGGCCCCUCCUCCUAACAACAGCGACAGCGUGACCUACAGCGUGAUCCAGAA |
| | GAGACCUAUGGGCGACUACGAGAACGUGAACCCUAGCUGCCCUGAGGACGAGAGCAUC |
| | CACUACAGCGAGCUGGUGCAGUUCGGCGCCGGCAAGAGACCUCAGGCCAAGGAGGACG |
| | UGGACUACGUGACCCUGAAGCAC |
| | [BCD15 nt. seq.] |
| 165 | AUGCCUAGCCCUCUGCCUGUGAGCCUGCUGCUGUUCCUGACCCUGGUGGGCGGCAGAC |
| | CUCAGAACAGCCUGCUGGUGGAGGUGGAGGAGGGCGACAACGUGGUGCUGAGCUGCCU |
| | GAGAGACAGCAGCCCUGUGAGCAGCGAGAAGCUGGCCUGGUACAGAGGCAACCAGAGC |
| | ACCCCUUUCCUGGAGCUGAGCCUGAGAAGCCCUGACCUGGGCCUGCACAUCGGCCCUCU |
| | GGGCAUCCUGCUGGUGAUCGUGAACGUGAGCGACCACAGAGGCGGCUUCUACCUGUGC |
| | CAGAAGCGGCCUAGCUUCAAGGACACCUGGCAGCCUGCCUGGACCGUGAACGUGGAGG |
| | ACAGCGGCGAGCUGUUCCGGUGGAACGCCAGCGACCUGGGCGACCUGGACUGCGACCU |
| | GGGCAACAGAAGCAGCGGCAGCCACAGAAGCACGAGUGGAUCUCAGCUGUACGUGUGG |
| | GCCACCGACCACCCUGAGGUGUGGAAGACCAAGCCUGUGUGCGCCCCUAGAGAGAUCA |
| | GCCUGAACCAGAGCCUGAUCAACCAGGACCUGACCGUGGCCCCUGGCAGCACCCUGUGG |
| | CUGAGCUGCGGCGUGCCUCCUGUGCCUGUGACCAAGGGCAGCAUCAGCUGGACCCACG |
| | UGCACCCUAAGACCCUGAACGUGAGCUUACUGUCCCUGAGCCUGGGCGGCGAGCACCC |
| | UGUGAGAGAGAUGUGGGUGUGGGGCUCACUCCUGUUGCUGCCUCAGGCCAAGGCCAGC |
| | GACGAGGGCACCUACUACUGCCUGCAGGGCGGCCUGACCAUCAAGAUGCACGUGAAGG |
| | UGAUCGCCAGAAGCGCCGUGUGGCUGUGGCUGCUGAGAACCGGCGGCUGGAUCGUGCC |
| | UGUGGUGACCCUGGUGUACGUGAUCUUCUGCAUGGUGAGCAUGGCCGCCUUCCUGUAC |
| | UUCGGCGGCGGCGGCUCUGGUGGCGGAGGAAGCUACAACCUGGCCAUGGACGACACCG |
| | UGAGCUACGCCGUGCUGAGAUUCCCUGAGAGCGACACUCACGGCGCCGGAGGAGCAAG |
| | AAGCCCUGCCACCCAGGGCCCUCCUCCUAACGACGACGACACCGUUACCUACAGCGUGC |
| | UGCAGAAGAGAAACAUGGGCGACUACGAGAACGUGAGCCCUAACUGCCCUGAGGACGA |
| | GAGCAUCCACUACAGCGAGCUGGUGCAGUUCGGCGCCGGCAAGCGACCUCAAGCGAAG |
| | GAGGACGUGGACUACGUGACCCUGAAGCAC |
| | [BCD16 nt. seq.] |
| 166 | AUGCUGGGCGGCCUGGGCGUGCUGAGAACCCUGCCUCUGCUACUUCUCUUCCUGAGCG |
| | AGGCCUGCCUGGGCCCUGGCUGCCAGGCCCUGAUGCUGGAGAGGGACCCUCCUAGCCU |
| | GACCGUGAACCUGGGCGAGGAGGCCGUGCUGACCUGCAAGAACGACGGCAAGAACCCU |
| | AACAUCACCUGGUGGUUCAGCCUGCAGAGCAACAGCACCUGGCCUCCUAUGCCUCUAG |
| | GUCCUGGAUUGGGCCCAAUGGGCAAGCUUAUCUUCCCUGAGGUGAACAAGAGCCACAG |
| | AGGCCUGUACUGGUGCCAGGUGAUCGAGAGCAAGGAGGUGAAGAGAAGCUGCGGCACC |
| | UACCUGAGAGUGAGAAAGCAGGUGCCUAGACCUUUCCUGGACAUGGGAGAAGGCACCA |
| | AGAACAGAAUCAUCACCGCCGAGGGCAUCAUCCUGUUAUUCUGCGCCGUGGUGCCUGG |
| | CACCCUGCUUCUGUUCAGAAAGCGGUGGCAGAACGAGAAGUUCGGCGUGUACAACCUG |
| | GCCAUGGACGACACCGUGAGCUACGCCGUCCUGCGAUUCCCUGAAAGCGACACACACG |
| | GCGCUGGCGGUGCCAGAAGCCCUGCCACCCAGGGCCCUCCUCCUAACGACGACGACACU |
| | GUUACCUACAGCGUGCUGCAGAAGAGAAAUAUGGGUGACUACGAGAACGUGAGCCCUA |
| | ACUGCCCUGAGGACGAGAGCAUCCACUACAGCGAGCUGGUGCAGUUCGGUGCUGGAAA |
| | GCGGCCUCAGGCCAAGGAGGACGUGGACUACGUGACCCUGAAGCAC |
| | [BCD17 nt. seq.] |
| 167 | AUGGCCACCCUGGUGCUGAGCCCUGUGCCUUGCCACUGGCUGAUGUUCCUGCUGCUCC |
| | UUUUGAGCGGCGAGCCUGUGCCUGCCAUGACCAAGAGCGACCAGCCUCCUAUCUUCCA |
| | GGGCAGCCCUUGCAGCAAGAUCUGGCAGCACCCUAGAUUCGCCGCCAAGAAGAGAAGC |
| | AGCAUGGUGAAGUUCCACUGCCACACCGACUACAGCGGCGUGAUGACCUGGUUCAGAC |
| | AGAAGGGCAACCAACGACCUCAGGAGCUGUUCCCUGAGGACGGCCACAUCAGCCAGAC |
| | CAGAAACGGCAGCGUGUACACCCUGACCCUGCAGAACAUCCAGUACGAGGACAACGGC |
| | AUCUACUUCUGCCAGCAGAAGUGCAACAGCACCGAGCCUGACGUGACCGACGGCUGCG |
| | GCACCGAGCUGCUGGUGCUGGGCUUCAGCACCCUGGACCAGCUGAAGAGAAGAAACAC |
| | CCUGAAGGACGGCAUCAUCAUGAUCCAGACCCUGCUGAUCAUCCUGUUCAUCAUCGUG |
| | CCUAUCUUCCUGCUGCUGGACAAGGACUACAACCUGGCCAUGGACGACACCGUGAGCU |
| | ACGCCGUGCUGAGAUUCCCUGAGAGCGACACGCACGGCGCCGGAGGCGCCAGAAGCCC |
| | UGCCACCCAGGGCCCUCCUCCUAACGACGACGAUACAGUUACCUACAGCGUGCUGCAGA |
| | AGAGAAACAUGGGCGACUACGAGAACGUGAGCCCUAACUGCCCUGAGGACGAGAGCAU |
| | CCACUACAGCGAGCUGGUGCAGUUCGGCGCCGGCAAGCGACCGCAGGCCAAGGAGGAC |
| | GUGGACUACGUGACCCUGAAGCAC |
| | [BCD18 nt. seq.] |
| 168 | MPGGPGVLQALPATIFLLFLLSAVYLGPGCQALWMHKVPASLMVSLGEDAHFQCPHNSSNNA |
| | NVTWWRVLHGNYTWPPEFLGPGEDPNGTLIIQNVNKSHGGIYVCRVQEGNESYQQSCGTYLR |
| | VRQPPPRPFLDMGEGTKNRIITAEGIILLFCAVVPGTLLLFRKRWQNEKLGLDAGDEYEDENLA |
| | EGLNLDDCSMAEDISRGLQGTYQDVGSLNIGDVQLEKPYNPMMEDGISYTTLRFPEMNIPRTG |
| | DAESSEMQRPPPDCDDTVTYSALHKRQVGDYENVIPDFPEDEGIHYSELIQFGVGERPQAQEN |
| | VDYVILKH |
| | [BCD1 a.a. seq.] |
| 169 | MPGGPGVLQALPATIFLLFLLSAVYLGPGCQALWMHKVPASLMVSLGEDAHFQCPHNSSNNA |
| | NVTWWRVLHGNYTWPPEFLGPGEDPNGTLIIQNVNKSHGGIYVCRVQEGNESYQQSCGTYLR |
| | VRQPPPRPFLDMGEGTKNRIITAEGIILLFCAVVPGTLLLFRKRWQNEKLGLYNPMMEDGISYT |
| | TLRFPEMNIPRTGDAESSEMQRPPPDCDDTVTYSALHKRQVGDYENVIPDFPEDEGIHYSELIQ |
| | FGVGERPQAQENVDYVILKH |
| | [BCD2 a.a. seq.] |
| 170 | MARLALSPVPSHWMVALLLLLSAEPVPAARSEDRYRNPKGSACSRIWQSPRFIARKRGFTVK |
| | MHCYMNSASGNVSWLWKQEMDENPQQLKLEKGRMEESQNESLATLTIQGIRFEDNGIYFCQ |
| | QKCNNTSEVYQGCGTELRVMGFSTLAQLKQRNTLKDGIIMIQTLLIILFIIVPIFLLLDKDDSKA |
| | GMEEDHTAEGLDIDQTATAEDIVTLRTGEVKWSVGEHPGQEYNPMMEDGISYTTLRFPEMNIP |
| | RTGDAESSEMQRPPPDCDDTVTYSALHKRQVGDYENVIPDFPEDEGIHYSELIQFGVGERPQA |
| | QENVDYVILKH |
| | [BCD3 a.a. seq.] |
| 171 | MARLALSPVPSHWMVALLLLLSAEPVPAARSEDRYRNPKGSACSRIWQSPRFIARKRGFTVK |
| | MHCYMNSASGNVSWLWKQEMDENPQQLKLEKGRMEESQNESLATLTIQGIRFEDNGIYFCQ |
| | QKCNNTSEVYQGCGTELRVMGFSTLAQLKQRNTLKDGIIMIQTLLIILFIIVPIFLLLDKDYNPM |
| | MEDGISYTTLRFPEMNIPRTGDAESSEMQRPPPDCDDTVTYSALHKRQVGDYENVIPDFPEDE |
| | GIHYSELIQFGVGERPQAQENVDYVILKH |
| | [BCD4 a.a. seq.] |
| 172 | MPPPRLLFFLLFLTPMEVRPEEPLVVKVEEGDNAVLQCLKGTSDGPTQQLTWSRESPLKPFLK |
| | LSLGLPGLGIHMRPLAIWLFIFNVSQQMGGFYLCQPGPPSEKAWQPGWTVNVEGSGELFRWN |
| | VSDLGGLGCGLKNRSSEGPSSPSGKLMSPKLYVWAKDRPEIWEGEPPCLPPRDSLNQSLSQDL |
| | TMAPGSTLWLSCGVPPDSVSRGPLSWTHVHPKGPKSLLSLELKDDRPARDMWVMETGLLLPR |
| | ATAQDAGKYYCHRGNLTMSFHLEITARPVLWHWLLRTGGWKVSAVTLAYLIFCLCSLVGILH |
| | LGGGGSGGGGSYNPMMEDGISYTTLRFPEMNIPRTGDAESSEMQRPPPDCDDTVTYSALHKR |
| | QVGDYENVIPDFPEDEGIHYSELIQFGVGERPQAQENVDYVILKH |
| | [BCD5 a.a. seq.] |
| 173 | MPPPRLLFFLLFLTPMEVRPEEPLVVKVEEGDNAVLQCLKGTSDGPTQQLTWSRESPLKPFLK |
| | LSLGLPGLGIHMRPLAIWLFIFNVSQQMGGFYLCQPGPPSEKAWQPGWTVNVEGSGELFRWN |
| | VSDLGGLGCGLKNRSSEGPSSPSGKLMSPKLYVWAKDRPEIWEGEPPCLPPRDSLNQSLSQDL |
| | TMAPGSTLWLSCGVPPDSVSRGPLSWTHVHPKGPKSLLSLELKDDRPARDMWVMETGLLLPR |
| | ATAQDAGKYYCHRGNLTMSFHLEITARPVLWHWLLRTGGWKVSAVTLAYLIFCLCSLVGILH |
| | LGGGGSGGGGSGGGGSFWEEFESLQKQEVKNLHQRLEGQRPENKGKNRYKNILPFDHSRVIL |
| | QGRDSNIPGSDYINANYIKNQLLGPDENAKTYIASQGCLEATVNDFWQMAWQENSRVIVMTT |
| | REVEKGRNKCVPYWPEVGMQRAYGPYSVTNCGEHDTTEYKLRTLQVSPLDNGDLIREIWHY |
| | QYLSWPDHGVPSEPGGVLSFLDQINQRQESLPHAGPIIVHCSAGIGRTGTIIVIDMLMENISTKG |
| | LDCDIDIQKTIQMVRAQRSGMVQTEAQYKFIYVAIAQF |
| | [BCD6 a.a. seq.] |
| 174 | MPPPRLLFFLLFLTPMEVRPEEPLVVKVEEGDNAVLQCLKGTSDGPTQQLTWSRESPLKPFLK |
| | LSLGLPGLGIHMRPLAIWLFIFNVSQQMGGFYLCQPGPPSEKAWQPGWTVNVEGSGELFRWN |
| | VSDLGGLGCGLKNRSSEGPSSPSGKLMSPKLYVWAKDRPEIWEGEPPCLPPRDSLNQSLSQDL |
| | TMAPGSTLWLSCGVPPDSVSRGPLSWTHVHPKGPKSLLSLELKDDRPARDMWVMETGLLLPR |
| | ATAQDAGKYYCHRGNLTMSFHLEITARPVLWHWLLRTGGWKVSAVTLAYLIFCLCSLVGILH |
| | LQRALVLRRKRKRMTDPTRRFFKVTPPPGSGPQNQAGNVLSLPTPTSGLGRAQRWAAGLGGT |
| | APSAGNPSSDVQADGALGSRSPPGVGPEEEEGEGAEEPDSEEDSEFYENDSNLGQDQLSQDGS |
| | GAENPEDEPLGPEDEDSFSNAESYENEDEELTQPVARTMDFLSPHGSAWDPSREATSLGSQSA |
| | EDMRGILYAAPQLRSIRGQPGPNHEEDADSYENMDNPDGPDPAWGGGGRMGTWSTRYNPM |
| | MEDGISYTTLRFPEMNIPRTGDAESSEMQRPPPDCDDTVTYSALHKRQVGDYENVIPDFPEDE |
| | GIHYSELIQFGVGERPQAQENVDYVILKH |
| | [BCD7 a.a. seq.] |
| 175 | MWFLTTLLLWVPVDGQVDTTKAVITLQPPWVSVFQEETVTLHCEVLHLPGSSSTQWFLNGTA |
| | TQTSTPSYRITSASVNDSGEYRCQRGLSGRSDPIQLEIHRGWLLLQVSSRVFTEGEPLALRCHA |
| | WKDKLVYNVLYYRNGKAFKFFHWNSNLTILKTNISHNGTYHCSGMGKHRYTSAGISVTVKE |
| | LFPAPVLNASVTSPLLEGNLVTLSCETKLLLQRPGLQLYFSFYMGSKTLRGRNTSSEYQILTAR |
| | REDSGLYWCEAATEDGNVLKRSPELELQVLGLQLPTPVWFHVLFYLAVGIMFLVNTVLWVTI |
| | VVALIYCRKKRISALPGYPECREMGETLPEKPANPTNPDEADKVGAENTITYSLLMHPDALEE |
| | PDDQNRI |
| | [BCD8 a.a. seq.] |
| 176 | MILTSFGDDMWLLTTLLLWVPVGGEVVNATKAVITLQPPWVSIFQKENVTLWCEGPHLPGDS |
| | STQWFINGTAVQISTPSYSIPEASFQDSGEYRCQIGSSMPSDPVQLQIHNDWLLLQASRRVLTEG |
| | EPLALRCHGWKNKLVYNVVFYRNGKSFQFSSDSEVAILKTNLSHSGIYHCSGTGRHRYTSAG |
| | VSITVKELFTTPVLRASVSSPFPEGSLVTLNCETNLLLQRPGLQLHFSFYVGSKILEYRNTSSEY |
| | HIARAEREDAGFYWCEVATEDSSVLKRSPELELQVLGPQSSAPVWFHILFYLSVGIMFSLNTVL |
| | YVVSLVYLKKKQVPALPGNPDHREMGETLPEEVGEYRQPSGGSVPVSPGPPSGLEPTSSSPYN |
| | PPDLEEAAKTEAENTITYSLLKHPEALDEETEHDYQNHI |
| | [BCD9 a.a. seq.] |
| 177 | MPGGLEALRALPLLLFLSYACLGPGCQALRVEGGPPSLTVNLGEEARLTCENNGRNPNITWWF |
| | SLQSNITWPPVPLGPGQGTTGQLFFPEVNKNHRGLYWCQVIENNILKRSCGTYLRVRNPVPRP |
| | FLDMGEGTKNRIITAEGIILLFCAVVPGTLLLFRKRWQNEKFGVDMPDDYEDENLAEGLNLDD |
| | CSMAEDISRGLQGTYQDVGNLHIGDAQLEKPYNPAMDDTVSYAILRFPESDTHNTGDAGTPA |
| | TQAPPPNNSDSVTYSVIQKRPMGDYENVNPSCPEDESIHYSELVQFGAGKRPQAKEDVDYVTL |
| | KH |
| | [BCD10 a.a. seq.] |
| 178 | MATLVLSSMPCHWLLFLLLLFSGEPVPAMTSSDLPLNFQGSPCSQIWQHPRFAAKKRSSMVKF |
| | HCYTNHSGALTWFRKRGSQQPQELVSEEGRIVQTQNGSVYTLTIQNIQYEDNGIYFCKQKCDS |
| | ANHNVTDSCGTELLVLGFSTLDQLKRRNTLKDGIILIQTLLIILFIIVPIFLLLDKDDGKAGMEED |
| | HTAEGLNIDQTATAEDIVTLRTGEVKWSVGEHPGQEYNPAMDDTVSYAILRFPESDTHNTGD |
| | AGTPATQAPPPNNSDSVTYSVIQKRPMGDYENVNPSCPEDESIHYSELVQFGAGKRPQAKEDV |
| | DYVTLKH |
| | [BCD11 a.a. seq.] |
| 179 | MPPPRLLFFLLFLTPMEVRPQKSLLVEVEEGGNVVLPCLPDSSPVSSEKLAWYRGNQSTPFLEL |
| | SPGSPGLGLHVGSLGILLVIVNVSDHMGGFYLCQKRPPFKDIWQPAWTVNVEDSGEMFRWNA |
| | SDVRDLDCDLRNRSSGSHRSTSGSQLYVWAKDHPKVWGTKPVCAPRGSSLNQSLINQDLTVA |
| | PGSTLWLSCGVPPVPVAKASISWTHVHPRRPNVSLLSLSLGGEHPVREMWVWGSLLLLPQAT |
| | ALDEGTYYCLRGNLTIERHVKVIARSAVWLWLLRTGGWIVPVVTLVYVIFCMVSLVAFLYCG |
| | GGGSGGGGSYNPAMDDTVSYAILRFPESDTHNTGDAGTPATQAPPPNNSDSVTYSVIQKRPM |
| | GDYENVNPSCPEDESIHYSELVQFGAGKRPQAKEDVDYVTLKH |
| | [BCD12 a.a. seq.] |
| 180 | MPSPLPVSFLLFLTLVGGRPQKSLLVEVEEGGNVVLPCLPDSSPVSSEKLAWYRGNQSTPFLEL |
| | SPGSPGLGLHVGSLGILLVIVNVSDHMGGFYLCQKRPPFKDIWQPAWTVNVEDSGEMFRWNA |
| | SDVRDLDCDLRNRSSGSHRSTSGSQLYVWAKDHPKVWGTKPVCAPRGSSLNQSLINQDLTVA |
| | PGSTLWLSCGVPPVPVAKASISWTHVHPRRPNVSLLSLSLGGEHPVREMWVWGSLLLLPQAT |
| | ALDEGTYYCLRGNLTIERHVKVIARSAVWLWLLRTGGWIVPVVTLVYVIFCMVSLVAFLYCQ |
| | RAFILRRKRKRMTDPARRFFKVTPPSGNGTQNQYGNVLSLPTSTSGQAHAQRWAAGLGSVPG |
| | SAGNPRIQVQDTGAQSHETGLEEEGEAAEEPDSEEGSEFYENDSNLGQDQVSQDGSGAENPED |
| | EPMGPEEEDSFSNAESYENADEELAQPVGRMMDFLSPHGSAWDPSREASSLGSQSAEDMRGI |
| | LYAAPQLHSIQSGPSHEEDADSYENMDKSDDLEPAWEGEGHMGTWGTTYNPAMDDTVSYAI |
| | LRFPESDTHNTGDAGTPATQAPPPNNSDSVTYSVIQKRPMGDYENVNPSCPEDESIHYSELVQF |
| | GAGKRPQAKEDVDYVTLKH |
| | [BCD13 a.a. seq.] |
| 181 | MPGGLEALRALPLLLFLSYACLGPGCQALRVEGGPPSLTVNLGEEARLTCENNGRNPNITWWF |
| | SLQSNITWPPVPLGPGQGTTGQLFFPEVNKNHRGLYWCQVIENNILKRSCGTYLRVRNPVPRP |
| | FLDMGEGTKNRIITAEGIILLFCAVVPGTLLLFRKRWQNEKFGVYNPAMDDTVSYAILRFPESD |
| | THNTGDAGTPATQAPPPNNSDSVTYSVIQKRPMGDYENVNPSCPEDESIHYSELVQFGAGKRP |
| | QAKEDVDYVTLKH |
| | [BCD14 a.a. seq.] |
| 182 | MATLVLSSMPCHWLLFLLLLFSGEPVPAMTSSDLPLNFQGSPCSQIWQHPRFAAKKRSSMVKF |
| | HCYTNHSGALTWFRKRGSQQPQELVSEEGRIVQTQNGSVYTLTIQNIQYEDNGIYFCKQKCDS |
| | ANHNVTDSCGTELLVLGFSTLDQLKRRNTLKDGIILIQTLLIILFIIVPIFLLLDKDYNPAMDDTV |
| | SYAILRFPESDTHNTGDAGTPATQAPPPNNSDSVTYSVIQKRPMGDYENVNPSCPEDESIHYSE |
| | LVQFGAGKRPQAKEDVDYVTLKH |
| | [BCD15 a.a. seq.] |
| 183 | MPSPLPVSLLLFLTLVGGRPQNSLLVEVEEGDNVVLSCLRDSSPVSSEKLAWYRGNQSTPFLEL |
| | SLRSPDLGLHIGPLGILLVIVNVSDHRGGFYLCQKRPSFKDTWQPAWTVNVEDSGELFRWNAS |
| | DLGDLDCDLGNRSSGSHRSTSGSQLYVWATDHPEVWKTKPVCAPREISLNQSLINQDLTVAP |
| | GSTLWLSCGVPPVPVTKGSISWTHVHPKTLNVSLLSLSLGGEHPVREMWVWGSLLLLPQAKA |
| | SDEGTYYCLQGGLTIKMHVKVIARSAVWLWLLRTGGWIVPVVTLVYVIFCMVSMAAFLYFG |
| | GGGSGGGGSYNLAMDDTVSYAVLRFPESDTHGAGGARSPATQGPPPNDDDTVTYSVLQKRN |
| | MGDYENVSPNCPEDESIHYSELVQFGAGKRPQAKEDVDYVTLKH |
| | [BCD16 a.a. seq.] |
| 184 | MLGGLGVLRTLPLLLLFLSEACLGPGCQALMLERDPPSLTVNLGEEAVLTCKNDGKNPNITW |
| | WFSLQSNSTWPPMPLGPGLGPMGKLIFPEVNKSHRGLYWCQVIESKEVKRSCGTYLRVRKQV |
| | PRPFLDMGEGTKNRIITAEGIILLFCAVVPGTLLLFRKRWQNEKFGVYNLAMDDTVSYAVLRF |
| | PESDTHGAGGARSPATQGPPPNDDDTVTYSVLQKRNMGDYENVSPNCPEDESIHYSELVQFG |
| | AGKRPQAKEDVDYVTLKH |
| | [BCD17 a.a. seq.] |
| 185 | MATLVLSPVPCHWLMFLLLLLSGEPVPAMTKSDQPPIFQGSPCSKIWQHPRFAAKKRSSMVKF |
| | HCHTDYSGVMTWFRQKGNQRPQELFPEDGHISQTRNGSVYTLTLQNIQYEDNGIYFCQQKCN |
| | STEPDVTDGCGTELLVLGFSTLDQLKRRNTLKDGIIMIQTLLIILFIIVPIFLLLDKDYNLAMDDT |
| | VSYAVLRFPESDTHGAGGARSPATQGPPPNDDDTVTYSVLQKRNMGDYENVSPNCPEDESIH |
| | YSELVQFGAGKRPQAKEDVDYVTLKH |
| | [BCD18 a.a. seq.] |
| 186 | GGGAAAUAAGAGAGAAAAGAAGAGUAAGAAGAAAUAUAAGACCCCGGCGCCGCCACC |
| | [5′ UTR] |
| 187 | UGAUAAUAGGCUGGAGCCUCGGUGGCCUAGCUUCUUGCCCCUUGGGCCUCCCCCCAGCC |
| | CCUCCUCCCCUUCCUGCACCCGUACCCCCGUGGUCUUUGAAUAAAGUCUGAGUGGGCGG |
| | C |
| | [3′ UTR] |
| 188 | (GGGS)n, wherein n = 1-4 |
| 189 | GGGAAAUAAGAGAGAAAAGAAGAGUAAGAAGAAAUAUAAGA |
| 190 | GGGAAAUAAGAGAGAAAAGAAGAGUAAGAAGAAAUAUAAGAGCCACC |
| | [5′ UTR] |
| 191 | GGGAAAUAAGAGAGAAAAGAAGAGUAAGAAGAAAUAUAAGACCCCGGCGCCACC |
| | [5′ UTR] |
| 192 | GCCA/GCC |
| | [K0 Traditional Kozak sequence] |
| 193 | GCCGCC |
| | [EK] |
| 194 | CCCCGGCGCC |
| | [V1] |
| 195 | CCCCGGC |
| | [V2] |
| 196 | CCR(A/G)CCAUGG (SEQ ID NO: 196), where R is a purine |
| | (adenine or guanine)[Kozak consensus sequence] |
| 197 | GGGAGAUCAGAGAGAAAAGAAGAGUAAGAAGAAAUAUAAGAGCCACC |
| | [5′ UTR] |
| 198 | GGGAGACAAGCUUGGCAUUCCGGUACUGUUGGUAAAGCCACC |
| | [5′ UTR] |
| 199 | GGGAGAUCAGAGAGAAAAGAAGAGUAAGAAGAAAUAUAAGAGCCACC |
| | [5′ UTR] |
| 200 | GGGAGACAAGCUUGGCAUUCCGGUACUGUUGGUAAAGCCACC |
| | [5′ UTR] |
| 201 | GGGAAUUAACAGAGAAAAGAAGAGUAAGAAGAAAUAUAAGAGCCACC |
| | [5′ UTR] |
| 202 | GGGAAAUUAGACAGAAAAGAAGAGUAAGAAGAAAUAUAAGAGCCACC |
| | [5′ UTR] |
| 203 | GGGAAAUAAGAGAGUAAAGAACAGUAAGAAGAAAUAUAAGAGCCACC |
| | [5′ UTR] |
| 204 | GGGAAAAAAGAGAGAAAAGAAGACUAAGAAGAAAUAUAAGAGCCACC |
| | [5′ UTR] |
| 205 | GGGAAAUAAGAGAGAAAAGAAGAGUAAGAAGAUAUAUAAGAGCCACC |
| | [5′ UTR] |
| 206 | GGGAAAUAAGAGACAAAACAAGAGUAAGAAGAAAUAUAAGAGCCACC |
| | [5′ UTR] |
| 207 | GGGAAAUUAGAGAGUAAAGAACAGUAAGUAGAAUUAAAAGAGCCACC |
| | [5′ UTR] |
| 208 | GGGAAAUAAGAGAGAAUAGAAGAGUAAGAAGAAAUAUAAGAGCCACC |
| | [5′ UTR] |
| 209 | GGGAAAUAAGAGAGAAAAGAAGAGUAAGAAGAAAAUUAAGAGCCACC |
| | [5′ UTR] |
| 210 | GGGAAAUAAGAGAGAAAAGAAGAGUAAGAAGAAAUUUAAGAGCCACC |
| | [5′ UTR] |
| 211 | UCAAGCUUUUGGACCCUCGUACAGAAGCUAAUACGACUCACUAUAGGGAAAUAAGAGA |
| | GAAAAGAAGAGUAAGAAGAAAUAUAAGAGCCACC |
| | [5′ UTR] |
| 212 | UGAUAAUAGUCCAUAAAGUAGGAAACACUACAGCUGGAGCCUCGGUGGCCAUGCUUCU |
| | UGCCCCUUGGGCCUCCCCCCAGCCCCUCCUCCCCUUCCUGCACCCGUACCCCCGUGGUC |
| | UUUGAAUAAAGUCUGAGUGGGCGGC |
| | [3′ UTR] |
| 213 | UGAUAAUAGGCUGGAGCCUCGGUGGCUCCAUAAAGUAGGAAACACUACACAUGCUUCU |
| | UGCCCCUUGGGCCUCCCCCCAGCCCCUCCUCCCCUUCCUGCACCCGUACCCCCGUGGUC |
| | UUUGAAUAAAGUCUGAGUGGGCGGC |
| | [3′ UTR] |
| 214 | UGAUAAUAGGCUGGAGCCUCGGUGGCCAUGCUUCUUGCCCCUUCCAUAAAGUAGGAAA |
| | CACUACAUGGGCCUCCCCCCAGCCCCUCCUCCCCUUCCUGCACCCGUACCCCCGUGGUC |
| | UUUGAAUAAAGUCUGAGUGGGCGGC |
| | [3′ UTR] |
| 215 | UGAUAAUAGGCUGGAGCCUCGGUGGCCAUGCUUCUUGCCCCUUGGGCCUCCCCCCAGU |
| | CCAUAAAGUAGGAAACACUACACCCCUCCUCCCCUUCCUGCACCCGUACCCCCGUGGUC |
| | UUUGAAUAAAGUCUGAGUGGGCGGC |
| | [3′ UTR] |
| 216 | UGAUAAUAGGCUGGAGCCUCGGUGGCCAUGCUUCUUGCCCCUUGGGCCUCCCCCCAGCC |
| | CCUCCUCCCCUUCUCCAUAAAGUAGGAAACACUACACUGCACCCGUACCCCCGUGGUCU |
| | UUGAAUAAAGUCUGAGUGGGCGGC |
| | [3′ UTR] |
| 217 | UGAUAAUAGGCUGGAGCCUCGGUGGCCAUGCUUCUUGCCCCUUGGGCCUCCCCCCAGCC |
| | CCUCCUCCCCUUCCUGCACCCGUACCCCCUCCAUAAAGUAGGAAACACUACAGUGGUCU |
| | UUGAAUAAAGUCUGAGUGGGCGGC |
| | [3′ UTR] |
| 218 | UGAUAAUAGGCUGGAGCCUCGGUGGCCAUGCUUCUUGCCCCUUGGGCCUCCCCCCAGCC |
| | CCUCCUCCCCUUCCUGCACCCGUACCCCCGUGGUCUUUGAAUAAAGUUCCAUAAAGUAG |
| | GAAACACUACACUGAGUGGGCGGC |
| | [3′ UTR] |
| 219 | UGAUAAUAGUCCAUAAAGUAGGAAACACUACAGCUGGAGCCUCGGUGGCCAUGCUUCU |
| | UGCCCCUUGGGCCUCCCCCCAGCCCCUCCUCCCCUUCCUGCACCCGUACCCCCCGCAUUA |
| | UUACUCACGGUACGAGUGGUCUUUGAAUAAAGUCUGAGUGGGCGGC |
| | [3′ UTR] |
| 220 | UGAUAAUAGUCCAUAAAGUAGGAAACACUACAGCUGGAGCCUCGGUGGCCUAGCUUCU |
| | UGCCCCUUGGGCCUCCCCCCAGCCCCUCCUCCCCUUCCUGCACCCGUACCCCCCGCAUUA |
| | UUACUCACGGUACGAGUGGUCUUUGAAUAAAGUCUGAGUGGGCGGC |
| | [3′ UTR] |
| 221 | UGAUAAUAGGCUGGAGCCUCGGUGGCCUAGCUUCUUGCCCCUUGGGCCUCCCCCCAGCC |
| | CCUCCUCCCCUUCCUGCACCCGUACCCCCUCCAUAAAGUAGGAAACACUACAGUGGUCU |
| | UUGAAUAAAGUCUGAGUGGGCGGC |
| | [3′ UTR] |
| 222 | TAATACGACTCACTATAGGGNNNNNNNNN |
| | [T7 promoter] |
| 223 | TAATACGACTCACTATAG |
| | [T7 promoter] |
| 224 | TAATACGACTCACTATAAGNNNNNNNNNN |
| | [T7 promoter] |
| 225 | ATTATGCTGAGTGATATTCNNNNNNNNNN |
| | [T7 promoter] |
| 226 | GSGATNFSLLKQAGDVEENPGP |
| | [2A peptide] |
| 227 | GGAAGCGGAGCTACTAACTTCAGCCTGCTGAAGCAGGCTGGAGACGTGGAGGAGAACCC |
| | TGGACCT |
| | [nucleic acid encoding 2A peptide] |
| 228 | TCCGGACTCAGATCCGGGGATCTCAAAATTGTCGCTCCTGTCAAACAAACTCTTAACTTTG |
| | ATTTACTCAAACTGGCTGGGGATGTAGAAAGCAATCCAGGTCCACTC |
| | [nucleic acid encoding 2A peptide] |
| 229 | MASSGMADSANHLPFFFGNITREEAEDYLVQGGMSDGLYLLRQSRNYLGGFALSVAHGRKA |
| | HHYTIERELNGTYAIAGGRTHASPADLCHYHSQESDGLVCLLKKPFNRPQGVQPKTGPFEDLK |
| | ENLIREYVKQTWNLQGQALEQAIISQKPQLEKLIATTAHEKMPWFHGKISREESEQIVLIGSKT |
| | NGKFLIRARDNNGSYALCLLHEGKVLHYRIDKDKTGKLSIPEGKKFDTLWQLVEHYSYKADG |
| | LLRVLTVPCQKIGTQGNVNFGGRPQLPGSHPATWSAGGIISRIKSYSFPKPGHRKSSPAQGNRQ |
| | ESTVSFNPYEPELAPWAADKGPQREALPMDTEVYESPYADPEEIRPKEVYLDRKLL |
| | [hSyk(nSH2-IA-cSH2-IB)] |
| 230 | MASSGMADSANHLPFFFGNITREEAEDYLVQGGMSDGLYLLRQSRNYLGGFALSVAHGRKA |
| | HHYTIERELNGTYAIAGGRTHASPADLCHYHSQESDGLVCLLKKPFNRPQGVQPKTGPFEDLK |
| | ENLIREYVKQTWNLQGQALEQAIISQKPQLEKLIATTAHEKMPWFHGKISREESEQIVLIGSKT |
| | NGKFLIRARDNNGSYALCLLHEGKVLHYRIDKDKTGKLSIPEGKKFDTLWQLVEHYSYKADG |
| | LLRVLTVPCGGGGSGGGGSGGGGS |
| | [hSyk(nSH2-IA-cSH2-GS)] |
| 231 | MASSGMADSANHLPFFFGNITREEAEDYLVQGGMSDGLYLLRQSRNYLGGFALSVAHGRKA |
| | HHYTIERELNGTYAIAGGRTHASPADLCHYHSQESDGLVCLLKKPFGGGGSGGGGSGGGGSG |
| | GGGSWFHGKISREESEQIVLIGSKTNGKFLIRARDNNGSYALCLLHEGKVLHYRIDKDKTGKLS |
| | IPEGKKFDTLWQLVEHYSYKADGLLRVLTVPCGGGGSGGGGSGGGGS |
| | [hSyk(nSH2-GS-cSH2-GS)] |
| 232 | ATGGCCAGCAGCGGCATGGCCGACAGCGCCAACCACCTGCCTTTCTTCTTCGGCAACATC |
| | ACCAGAGAGGAGGCCGAGGACTACCTGGTGCAGGGCGGCATGAGCGACGGCCTGTACCT |
| | GCTGAGACAGAGCAGAAACTACCTGGGCGGCTTCGCCCTGAGCGTGGCCCACGGCAGAA |
| | AGGCCCACCACTACACCATCGAGAGAGAGCTGAACGGCACCTACGCCATCGCCGGCGGC |
| | AGAACCCACGCCAGCCCTGCCGACCTGTGCCACTACCACAGCCAGGAGTCAGACGGCCTG |
| | GTGTGCCTGCTGAAGAAGCCTTTCAACAGACCTCAGGGCGTGCAGCCTAAGACCGGCCCT |
| | TTCGAGGACCTGAAGGAGAACCTGATCAGAGAGTACGTGAAGCAGACCTGGAACCTGCA |
| | GGGCCAGGCCCTGGAGCAGGCCATCATCAGCCAGAAGCCTCAGCTGGAGAAGCTGATCG |
| | CCACCACCGCCCACGAGAAGATGCCTTGGTTCCACGGCAAGATCTCAAGGGAGGAGTCGG |
| | AGCAGATCGTGCTGATCGGCAGCAAGACCAACGGCAAGTTCCTCATTAGAGCCAGAGAC |
| | AACAACGGCAGCTACGCCCTGTGTCTCCTGCACGAGGGCAAGGTGCTGCACTACAGAATC |
| | GACAAGGACAAGACCGGCAAGCTGAGCATCCCTGAGGGCAAGAAGTTCGACACCCTGTG |
| | GCAGCTGGTGGAGCACTACAGCTACAAGGCAGACGGACTGCTGAGAGTGCTGACCGTGC |
| | CTTGCCAGAAGATCGGCACCCAGGGCAACGTGAACTTCGGCGGTAGACCACAGCTGCCTG |
| | GCAGCCACCCTGCCACCTGGTCTGCCGGTGGCATTATCAGCAGAATCAAGAGCTACAGCT |
| | TCCCTAAGCCTGGCCACAGAAAGAGTTCCCCAGCACAAGGTAACAGACAGGAGAGCACC |
| | GTGAGCTTCAACCCTTACGAGCCTGAGCTGGCCCCTTGGGCCGCCGACAAGGGCCCTCAG |
| | AGAGAGGCCCTGCCTATGGACACCGAGGTGTACGAGAGCCCTTACGCCGACCCTGAGGA |
| | GATCAGACCTAAGGAGGTGTACCTGGACAGAAAGCTGCTGTTCTGGGAGGAGTTCGAGAG |
| | CCTGCAGAAGCAGGAGGTGAAGAACCTGCACCAGAGACTGGAGGGCCAAAGGCCTGAGA |
| | ACAAGGGAAAGAACAGATACAAGAACATATTACCTTTCGACCACAGCAGAGTGATTCTGC |
| | AGGGCAGAGACAGCAACATCCCTGGCAGCGACTACATCAACGCCAACTACATTAAGAAC |
| | CAGCTGCTGGGCCCTGACGAGAACGCCAAGACCTACATCGCCAGCCAGGGCTGCCTGGAG |
| | GCCACCGTGAACGACTTCTGGCAGATGGCGTGGCAGGAGAACTCAAGAGTGATCGTGATG |
| | ACCACCCGGGAGGTGGAGAAGGGCAGAAACAAGTGCGTGCCTTACTGGCCTGAGGTGGG |
| | CATGCAGAGAGCCTACGGCCCTTACAGCGTGACCAACTGCGGCGAGCACGACACCACCG |
| | AGTACAAGCTGAGAACCCTGCAGGTGAGCCCTCTGGACAACGGCGATCTGATCAGGGAG |
| | ATCTGGCACTACCAGTATTTGTCCTGGCCTGACCACGGCGTGCCTAGCGAGCCTGGCGGC |
| | GTGCTGAGCTTCCTGGACCAGATCAACCAGCGTCAAGAAAGTCTCCCTCACGCCGGCCCT |
| | ATCATCGTGCACTGCAGCGCCGGCATCGGAAGGACCGGCACCATCATCGTGATCGACATG |
| | CTGATGGAGAACATCAGCACCAAGGGCCTGGACTGCGACATCGACATCCAGAAGACCAT |
| | CCAGATGGTGAGAGCCCAGAGATCCGGCATGGTGCAGACCGAGGCCCAGTACAAGTTCA |
| | TCTACGTGGCCATCGCCCAGTTC |
| | [BCD19 n.t. seq.] |
| 233 | ATGGCCAGCAGCGGCATGGCCGACAGCGCCAACCACCTGCCTTTCTTCTTCGGCAACATC |
| | ACCAGAGAGGAGGCCGAGGACTACCTGGTGCAGGGCGGCATGAGCGACGGCCTGTACCT |
| | GCTGAGACAGAGCAGAAACTACCTGGGCGGCTTCGCCCTGAGCGTGGCCCACGGCAGAA |
| | AGGCCCACCACTACACCATCGAGAGAGAGCTGAACGGCACCTACGCCATCGCCGGCGGC |
| | AGAACCCACGCCAGCCCTGCCGACCTGTGCCACTACCACAGCCAGGAGTCAGACGGCCTG |
| | GTGTGCCTGCTGAAGAAGCCTTTCAACAGACCTCAGGGCGTGCAGCCTAAGACCGGCCCT |
| | TTCGAGGACCTGAAGGAGAACCTGATCAGAGAGTACGTGAAGCAGACCTGGAACCTGCA |
| | GGGCCAGGCCCTGGAGCAGGCCATCATCAGCCAGAAGCCTCAGCTGGAGAAGCTGATCG |
| | CCACCACCGCCCACGAGAAGATGCCTTGGTTCCACGGCAAGATCAGCAGAGAGGAGAGC |
| | GAGCAGATCGTGCTGATCGGCAGCAAGACCAACGGCAAGTTCCTCATCAGAGCCAGAGA |
| | CAACAACGGCAGCTACGCCCTGTGTTTGCTGCACGAGGGCAAGGTGCTGCACTACAGAAT |
| | CGACAAGGACAAGACCGGCAAGCTGAGCATCCCTGAGGGCAAGAAGTTCGACACCCTGT |
| | GGCAGCTGGTGGAGCACTACAGCTACAAGGCCGACGGCCTGCTGAGAGTGCTGACCGTGC |
| | CTTGCGGAGGCGGAGGCAGCGGAGGTGGAGGTTCAGGAGGCGGCGGAAGCTTCTGGGAG |
| | GAGTTCGAGAGCCTGCAGAAGCAGGAGGTGAAGAACCTGCACCAGAGACTGGAGGGCCA |
| | GCGGCCTGAGAACAAGGGCAAGAACAGATACAAGAACATCCTGCCTTTCGACCACAGCA |
| | GAGTGATCCTGCAGGGCAGAGACAGCAACATCCCTGGCAGCGACTACATCAACGCCAACT |
| | ACATCAAGAACCAGCTGCTGGGCCCTGACGAGAACGCCAAGACCTACATCGCCAGCCAG |
| | GGCTGCCTGGAGGCCACCGTGAACGACTTCTGGCAAATGGCTTGGCAGGAGAATTCTCGC |
| | GTGATCGTGATGACGACCCGAGAGGTGGAGAAGGGCAGAAACAAGTGCGTGCCTTACTG |
| | GCCTGAGGTGGGCATGCAGAGAGCCTACGGCCCTTACAGCGTGACCAACTGCGGCGAGC |
| | ACGACACCACCGAGTACAAGCTGAGAACCCTGCAGGTGAGCCCTCTGGACAACGGCGAT |
| | CTTATTCGGGAGATCTGGCACTACCAGTACCTGAGCTGGCCTGACCACGGCGTGCCTAGC |
| | GAGCCTGGCGGCGTGCTGAGCTTCCTGGACCAGATCAACCAGAGACAGGAGAGCCTGCCT |
| | CACGCCGGCCCTATCATCGTGCACTGCAGCGCCGGCATCGGCAGAACCGGCACCATCATC |
| | GTGATCGACATGCTGATGGAGAACATCAGCACCAAGGGCCTGGACTGCGACATCGACATC |
| | CAGAAGACCATCCAGATGGTGAGAGCCCAGAGAAGCGGCATGGTGCAGACCGAGGCCCA |
| | GTACAAGTTCATCTACGTGGCCATCGCCCAGTTC |
| | [BCD20 n.t. seq.] |
| 234 | ATGGCCAGCAGCGGCATGGCCGACAGCGCCAACCACCTGCCTTTCTTCTTCGGCAACATC |
| | ACCAGAGAGGAGGCCGAGGACTACCTGGTGCAGGGCGGCATGAGCGACGGCCTGTACCT |
| | GCTGAGACAGAGCAGAAACTACCTGGGCGGCTTCGCCCTGAGCGTGGCCCACGGCAGAA |
| | AGGCCCACCACTACACCATCGAGAGAGAGCTGAACGGCACCTACGCCATCGCCGGCGGC |
| | AGAACCCACGCCAGCCCTGCCGACCTGTGCCACTACCACAGCCAGGAGAGCGACGGTCTG |
| | GTGTGCCTGCTGAAGAAGCCTTTCGGAGGCGGAGGCAGCGGAGGAGGAGGCTCCGGTGG |
| | CGGTGGCAGTGGCGGCGGCGGAAGCTGGTTCCACGGCAAGATCTCTAGGGAAGAATCTG |
| | AGCAGATCGTGCTGATCGGCAGCAAGACCAACGGCAAGTTCCTGATCAGAGCCAGAGAC |
| | AACAACGGCAGCTACGCCCTGTGCTTACTGCACGAGGGCAAGGTGCTGCACTACAGAATC |
| | GACAAGGACAAGACCGGCAAGCTGAGCATCCCTGAGGGCAAGAAGTTCGACACCCTGTG |
| | GCAGCTGGTGGAGCACTACAGCTACAAGGCCGACGGACTGTTGAGAGTGCTGACCGTGCC |
| | TTGCGGAGGCGGCGGATCTGGAGGTGGTGGCTCAGGTGGCGGAGGCTCTTTCTGGGAGGA |
| | GTTCGAGAGCCTGCAGAAGCAGGAGGTGAAGAACCTGCACCAGAGACTGGAGGGCCAGC |
| | GGCCTGAGAACAAGGGAAAGAACAGATACAAGAACATCCTGCCATTCGACCACAGCAGA |
| | GTGATCCTGCAGGGCAGAGACAGCAACATCCCTGGCAGCGACTACATCAACGCCAATTAC |
| | ATAAAGAACCAGCTGCTGGGCCCTGACGAGAACGCCAAGACCTACATCGCCAGCCAGGG |
| | CTGCCTGGAGGCCACCGTGAACGACTTCTGGCAGATGGCCTGGCAGGAGAATAGCCGTGT |
| | GATCGTGATGACGACACGGGAGGTGGAGAAGGGCAGAAACAAGTGCGTGCCTTACTGGC |
| | CTGAGGTGGGCATGCAGAGAGCCTACGGCCCTTACAGCGTGACCAACTGCGGCGAGCAC |
| | GACACCACCGAGTACAAGCTGAGAACCCTGCAGGTGAGCCCTCTGGACAACGGCGACCTC |
| | ATCCGCGAGATCTGGCACTACCAGTACCTGAGCTGGCCTGACCACGGCGTGCCTAGCGAG |
| | CCTGGCGGCGTGCTGAGCTTCCTGGACCAGATCAACCAGAGACAGGAATCCCTCCCTCAC |
| | GCCGGCCCTATCATCGTGCACTGCAGCGCCGGCATCGGAAGGACCGGCACCATCATCGTG |
| | ATCGACATGCTGATGGAGAACATCAGCACCAAGGGCCTGGACTGCGACATCGACATCCAG |
| | AAGACCATCCAGATGGTGAGAGCCCAGAGATCTGGAATGGTGCAGACCGAGGCCCAGTA |
| | CAAGTTCATCTACGTGGCCATCGCCCAGTTC |
| | [BCD21 n.t. seq.] |
| 235 | ATGCCTCCTCCTAGACTGCTGTTCTTCCTTCTGTTCCTGACCCCTATGGAGGTGAGACCTGA |
| | GGAGCCTCTGGTGGTGAAGGTGGAGGAGGGCGACAACGCCGTGCTGCAGTGCCTGAAGG |
| | GCACCAGCGACGGCCCTACCCAGCAGCTGACCTGGAGCAGAGAGAGCCCTCTGAAGCCTT |
| | TCCTGAAGCTGAGCCTGGGCCTGCCTGGCCTGGGCATCCACATGCGTCCTCTGGCCATCTG |
| | GCTGTTCATCTTCAACGTGAGCCAGCAGATGGGCGGCTTCTACCTGTGCCAGCCTGGCCCT |
| | CCTAGCGAGAAGGCCTGGCAGCCAGGTTGGACCGTGAACGTGGAGGGCAGCGGCGAGCT |
| | GTTCCGGTGGAACGTGAGCGACCTGGGCGGCCTGGGTTGCGGCCTGAAGAACAGAAGCA |
| | GCGAGGGCCCTAGCAGCCCTAGCGGCAAGCTGATGAGCCCTAAGCTGTACGTGTGGGCCA |
| | AGGACAGACCTGAGATCTGGGAGGGAGAGCCTCCTTGCCTGCCTCCACGCGACAGCCTGA |
| | ACCAGAGCCTGAGCCAGGACCTGACCATGGCCCCTGGCTCTACCCTGTGGCTGAGCTGCG |
| | GCGTGCCTCCTGACAGCGTGAGCAGAGGCCCTTTGAGCTGGACCCACGTGCACCCTAAGG |
| | GACCAAAGAGCCTTCTGTCGCTGGAGCTGAAGGACGATCGTCCAGCCAGAGACATGTGGG |
| | TGATGGAGACAGGCCTGCTGCTGCCTAGAGCCACCGCCCAGGACGCCGGCAAGTACTACT |
| | GCCACAGAGGCAACCTCACCATGAGCTTCCACCTGGAGATCACCGCCAGACCTGTGCTGT |
| | GGCACTGGCTGCTGAGAACCGGCGGCTGGAAGGTGAGCGCCGTGACCCTGGCCTACCTGA |
| | TCTTCTGCCTGTGTAGCCTCGTGGGAATACTGCACCTTGGCGGAGGTGGTAGTGGTGGCGG |
| | CGGCTCTATGAGCGCCATCCAGGCCGCTTGGCCAAGTGGTACCGAGTGCATCGCCAAGTA |
| | CAACTTCCACGGCACCGCCGAGCAGGATCTACCTTTCTGCAAGGGCGACGTGCTGACCAT |
| | CGTGGCCGTAACCAAGGACCCTAACGCCTACAAGGCCAAGAACAAGGTGGGCAGAGAGG |
| | GCATCATCCCTGCCAACTACGTGCAGAAGCGGGAGGGTGTGAAGGCCGGCACCAAGCTGT |
| | CACTGATGCCTTGGTTCCACGGAAAGATCACCAGAGAGCAGGCCGAGAGGCTATTGTATC |
| | CGCCTGAAACTGGCCTTTTCCTTGTCAAGGAGAGCACCAACTACCCTGGCGACTACACGC |
| | TCTGCGTTTCCTGTGACGGCAAGGTGGAACACTACAGAATCATGTACCACGCCTCCAAGC |
| | TATCTATCGACGAGGAGGTGTACTTCGAGAACCTGATGCAGCTGGTGGCCCACTACACGA |
| | GCGACGCCGACGGCCTGTGCACCAGACTGATCAAGCCTAAGGTCATGGAAGGCACCGTTG |
| | CCGCTCAGGACGAGTTCTACCGGTCCGGCTGGGCCCTGAACATGAAGGAGTTAAAGCTCC |
| | TGCAGACCATCGGAAAGGGAGAGTTCGGCGACGTCATGCTGGGTGATTACAGAGGAAAT |
| | AAGGTTGCTGTGAAGTGCATCAAGAACGACGCAACCGCCCAAGCCTTCCTGGCCGAGGCC |
| | AGCGTGATGACCCAGCTGAGACACAGCAACCTGGTGCAGCTCTTGGGAGTGATCGTCGAG |
| | GAGAAGGGAGGCCTGTACATCGTGACCGAGTACATGGCCAAGGGAAGCTTAGTGGACTA |
| | CCTGCGTTCCAGGGGCCGTTCCGTTCTCGGAGGTGACTGCCTCTTAAAGTTCAGCCTGGAC |
| | GTGTGCGAGGCCATGGAGTACCTGGAAGGAAACAACTTCGTGCACCGTGACCTGGCCGCC |
| | AGAAACGTGCTGGTGAGCGAGGACAACGTAGCTAAGGTGTCTGATTTCGGCCTGACTAAG |
| | GAGGCCAGTTCTACTCAGGACACCGGTAAGCTCCCGGTAAAGTGGACCGCCCCTGAGGCC |
| | CTGAGAGAGAAGAAGTTCAGTACCAAGAGCGACGTGTGGAGCTTCGGCATCCTGCTGTGG |
| | GAGATCTACAGTTTCGGCAGAGTGCCTTACCCTAGAATTCCATTAAAGGACGTGGTACCT |
| | AGGGTTGAGAAGGGCTACAAGATGGACGCCCCGGACGGCTGCCCTCCTGCCGTGTACGAG |
| | GTGATGAAGAACTGCTGGCACCTGGACGCCGCCATGCGGCCGAGCTTCCTGCAGCTGAGG |
| | GAACAACTGGAGCACATCAAGACCCACGAGCTCCATCTG |
| | [BCD22 n.t. seq.] |
| 236 | ATGCCTGGCGGCCCTGGCGTGCTGCAGGCCCTGCCTGCCACCATCTTCCTGCTGTTCCTCC |
| | TGAGCGCCGTGTACCTGGGACCTGGCTGCCAGGCCCTGTGGATGCACAAGGTCCCAGCAA |
| | GCCTGATGGTGAGCCTGGGCGAGGACGCCCACTTCCAGTGCCCTCACAACAGCAGCAACA |
| | ACGCCAACGTGACCTGGTGGAGAGTGCTGCACGGCAACTACACCTGGCCTCCAGAATTCC |
| | TCGGCCCAGGCGAAGATCCTAACGGCACCCTGATCATCCAGAACGTGAACAAGAGCCAC |
| | GGCGGCATCTACGTGTGCAGAGTGCAGGAGGGCAACGAGAGCTACCAGCAGAGCTGCGG |
| | CACCTACCTGAGAGTGAGACAGCCTCCTCCTAGACCTTTCCTGGACATGGGTGAGGGCAC |
| | CAAGAACAGAATCATCACCGCCGAGGGCATCATTTTACTCTTCTGCGCCGTGGTGCCTGGT |
| | ACCCTATTATTGTTCAGAAAGAGGTGGCAGAACGAGAAGCTGGGTCTGGGCGGTGGAGGC |
| | AGCGGCATGAGCGCCATCCAGGCCGCCTGGCCTAGCGGCACCGAGTGCATCGCCAAGTAC |
| | AACTTCCACGGAACTGCCGAGCAGGACCTGCCTTTCTGCAAGGGCGACGTGCTGACCATC |
| | GTGGCCGTGACCAAGGACCCAAACGCCTACAAGGCTAAGAATAAGGTGGGCAGAGAGGG |
| | TATTATTCCTGCCAACTACGTGCAGAAGAGGGAAGGCGTGAAGGCCGGCACTAAGCTGAG |
| | TCTTATGCCTTGGTTCCACGGTAAGATCACCAGAGAGCAGGCCGAGAGACTGCTGTACCC |
| | ACCTGAAACCGGCTTGTTCCTGGTGAAGGAGAGCACCAACTATCCAGGCGACTACACCCT |
| | GTGCGTGAGCTGCGACGGCAAGGTGGAGCACTATCGCATCATGTACCACGCCTCAAAGTT |
| | GTCCATCGACGAGGAGGTGTACTTCGAGAACCTGATGCAGCTGGTGGCCCACTACACCAG |
| | CGACGCCGACGGCCTGTGCACCAGACTGATCAAGCCTAAGGTGATGGAAGGCACCGTGG |
| | CCGCCCAGGACGAGTTCTACAGAAGCGGTTGGGCGCTGAACATGAAGGAGCTGAAGCTG |
| | CTGCAGACCATCGGAAAGGGTGAGTTCGGTGACGTGATGCTGGGAGATTATAGAGGCAAC |
| | AAGGTGGCCGTAAAGTGCATCAAGAACGACGCCACAGCCCAGGCCTTCCTGGCCGAGGC |
| | CAGCGTGATGACCCAGCTGAGACACAGCAACCTGGTGCAGTTATTGGGCGTGATCGTGGA |
| | GGAGAAGGGCGGCCTGTACATCGTGACCGAGTACATGGCCAAGGGCTCTCTTGTGGACTA |
| | CCTCCGTAGCAGAGGCAGAAGCGTTCTTGGAGGTGACTGCCTGCTGAAGTTCAGCCTGGA |
| | CGTGTGCGAGGCCATGGAGTACCTAGAAGGTAATAACTTCGTGCACAGGGACCTGGCCGC |
| | CAGAAACGTGCTGGTGAGCGAGGACAACGTAGCCAAGGTTAGCGACTTCGGCCTGACGA |
| | AGGAAGCAAGTAGCACCCAGGACACCGGCAAGCTGCCTGTGAAGTGGACCGCCCCTGAG |
| | GCCCTGAGAGAGAAGAAGTTCTCCACAAAGAGCGACGTGTGGAGCTTCGGCATACTGCTG |
| | TGGGAGATCTACTCATTCGGCCGAGTTCCTTACCCTAGAATCCCTCTGAAGGACGTGGTCC |
| | CTAGAGTCGAGAAGGGATACAAGATGGACGCTCCTGACGGCTGCCCTCCTGCAGTCTACG |
| | AGGTGATGAAGAACTGCTGGCACCTGGACGCCGCCATGCGCCCTAGCTTCCTGCAACTTA |
| | GGGAGCAGCTGGAGCACATCAAGACCCACGAGCTGCACCTG |
| | [BCD23 n.t. seq.] |
| 237 | ATGGCCAGACTGGCCCTGTCACCTGTGCCTAGCCACTGGATGGTGGCCCTCCTGCTGCTGC |
| | TGTCTGCTGAACCAGTGCCTGCCGCCAGAAGCGAGGACAGATACAGAAACCCTAAAGGG |
| | AGCGCCTGCAGCAGAATCTGGCAGAGCCCGAGATTCATCGCCAGAAAGAGAGGCTTCAC |
| | CGTGAAGATGCACTGCTACATGAATAGCGCAAGCGGCAACGTGAGCTGGCTGTGGAAGC |
| | AGGAGATGGACGAGAACCCGCAGCAGCTGAAGCTGGAGAAGGGCAGAATGGAGGAGAG |
| | CCAGAACGAGAGCCTGGCTACCCTGACCATTCAAGGAATCAGATTCGAGGACAACGGCAT |
| | CTACTTCTGCCAGCAGAAGTGCAACAACACCAGCGAGGTGTACCAGGGCTGCGGCACCGA |
| | ACTGAGAGTGATGGGCTTCAGCACCCTGGCCCAACTTAAGCAGAGAAACACCCTGAAGG |
| | ACGGCATCATCATGATCCAGACCCTGCTGATCATCCTGTTCATCATCGTGCCTATCTTCCT |
| | GCTGCTGGACAAGGACGGCGGCGGCGGTAGCATGAGCGCCATCCAGGCCGCCTGGCCTA |
| | GCGGCACCGAGTGCATCGCCAAGTACAACTTCCACGGCACCGCCGAGCAGGACCTGCCTT |
| | TCTGCAAGGGCGACGTGCTCACCATCGTCGCCGTTACCAAGGACCCTAACGCCTACAAGG |
| | CCAAGAACAAGGTGGGCAGAGAGGGAATCATTCCTGCCAACTACGTGCAGAAGAGAGAG |
| | GGCGTGAAGGCCGGCACCAAGCTAAGCCTGATGCCTTGGTTCCACGGCAAGATCACAAGA |
| | GAGCAGGCCGAGAGACTGCTGTACCCTCCTGAGACTGGCCTGTTCCTGGTGAAGGAAAGC |
| | ACCAACTACCCTGGCGACTACACCCTGTGCGTGAGCTGCGACGGCAAGGTGGAGCACTAC |
| | CGAATCATGTACCACGCCAGCAAGCTGAGCATCGACGAGGAGGTGTACTTCGAGAACCTG |
| | ATGCAGCTGGTGGCCCACTACACCTCTGACGCCGACGGCCTGTGCACCAGACTGATCAAG |
| | CCAAAGGTGATGGAGGGCACCGTGGCCGCCCAGGACGAGTTCTACAGATCAGGCTGGGC |
| | CCTTAACATGAAGGAGTTGAAGCTGCTGCAGACCATCGGCAAGGGCGAGTTCGGCGACGT |
| | GATGCTGGGCGACTACAGAGGCAACAAGGTGGCCGTGAAGTGCATCAAGAACGACGCCA |
| | CCGCCCAAGCCTTCCTGGCCGAGGCCAGCGTGATGACCCAGCTGCGACACAGCAATCTGG |
| | TGCAGCTGCTGGGCGTGATCGTGGAGGAGAAGGGCGGCCTGTACATCGTGACTGAGTACA |
| | TGGCCAAGGGCAGCCTGGTGGATTATCTGAGATCAAGGGGCAGAAGCGTGCTGGGCGGC |
| | GACTGCCTGCTGAAATTCAGCCTGGACGTCTGCGAAGCCATGGAGTACCTGGAGGGGAAC |
| | AACTTCGTGCACCGCGACCTGGCCGCCAGAAACGTGCTGGTGTCCGAGGACAACGTGGCT |
| | AAAGTGAGCGACTTCGGCCTGACCAAGGAGGCCAGCAGCACCCAGGACACCGGCAAGCT |
| | GCCTGTGAAGTGGACCGCCCCTGAGGCCCTGAGAGAGAAGAAGTTCAGCACCAAGAGCG |
| | ACGTGTGGAGCTTCGGCATTCTGCTGTGGGAGATTTACAGCTTTGGCAGAGTGCCTTACCC |
| | TAGAATCCCTCTCAAAGACGTGGTGCCTAGAGTGGAGAAGGGCTACAAGATGGACGCCCC |
| | TGACGGCTGCCCTCCTGCCGTGTACGAGGTGATGAAGAACTGCTGGCACCTGGACGCCGC |
| | CATGCGGCCTAGCTTCTTACAGCTGAGAGAGCAGCTGGAGCACATCAAGACCCACGAACT |
| | GCACCTG |
| | [BCD24 n.t. seq.] |
| 238 | MASSGMADSANHLPFFFGNITREEAEDYLVQGGMSDGLYLLRQSRNYLGGFALSVAHGRKA |
| | HHYTIERELNGTYAIAGGRTHASPADLCHYHSQESDGLVCLLKKPFNRPQGVQPKTGPFEDLK |
| | ENLIREYVKQTWNLQGQALEQAIISQKPQLEKLIATTAHEKMPWFHGKISREESEQIVLIGSKT |
| | NGKFLIRARDNNGSYALCLLHEGKVLHYRIDKDKTGKLSIPEGKKFDTLWQLVEHYSYKADG |
| | LLRVLTVPCQKIGTQGNVNFGGRPQLPGSHPATWSAGGIISRIKSYSFPKPGHRKSSPAQGNRQ |
| | ESTVSFNPYEPELAPWAADKGPQREALPMDTEVYESPYADPEEIRPKEVYLDRKLLFWEEFES |
| | LQKQEVKNLHQRLEGQRPENKGKNRYKNILPFDHSRVILQGRDSNIPGSDYINANYIKNQLLG |
| | PDENAKTYIASQGCLEATVNDFWQMAWQENSRVIVMTTREVEKGRNKCVPYWPEVGMQRA |
| | YGPYSVTNCGEHDTTEYKLRTLQVSPLDNGDLIREIWHYQYLSWPDHGVPSEPGGVLSFLDQI |
| | NQRQESLPHAGPIIVHCSAGIGRTGTIIVIDMLMENISTKGLDCDIDIQKTIQMVRAQRSGMVQT |
| | EAQYKFIYVAIAQF |
| 239 | MASSGMADSANHLPFFFGNITREEAEDYLVQGGMSDGLYLLRQSRNYLGGFALSVAHGRKA |
| | HHYTIERELNGTYAIAGGRTHASPADLCHYHSQESDGLVCLLKKPFNRPQGVQPKTGPFEDLK |
| | ENLIREYVKQTWNLQGQALEQAIISQKPQLEKLIATTAHEKMPWFHGKISREESEQIVLIGSKT |
| | NGKFLIRARDNNGSYALCLLHEGKVLHYRIDKDKTGKLSIPEGKKFDTLWQLVEHYSYKADG |
| | LLRVLTVPCGGGGSGGGGSGGGGSFWEEFESLQKQEVKNLHQRLEGQRPENKGKNRYKNILP |
| | FDHSRVILQGRDSNIPGSDYINANYIKNQLLGPDENAKTYIASQGCLEATVNDFWQMAWQEN |
| | SRVIVMTTREVEKGRNKCVPYWPEVGMQRAYGPYSVTNCGEHDTTEYKLRTLQVSPLDNGD |
| | LIREIWHYQYLSWPDHGVPSEPGGVLSFLDQINQRQESLPHAGPIIVHCSAGIGRTGTIIVIDML |
| | MENISTKGLDCDIDIQKTIQMVRAQRSGMVQTEAQYKFIYVAIAQF |
| | [BCD20 a.a. seq.] |
| 240 | MASSGMADSANHLPFFFGNITREEAEDYLVQGGMSDGLYLLRQSRNYLGGFALSVAHGRKA |
| | HHYTIERELNGTYAIAGGRTHASPADLCHYHSQESDGLVCLLKKPFGGGGSGGGGSGGGGSG |
| | GGGSWFHGKISREESEQIVLIGSKTNGKFLIRARDNNGSYALCLLHEGKVLHYRIDKDKTGKLS |
| | IPEGKKFDTLWQLVEHYSYKADGLLRVLTVPCGGGGSGGGGSGGGGSFWEEFESLQKQEVK |
| | NLHQRLEGQRPENKGKNRYKNILPFDHSRVILQGRDSNIPGSDYINANYIKNQLLGPDENAKT |
| | YIASQGCLEATVNDFWQMAWQENSRVIVMTTREVEKGRNKCVPYWPEVGMQRAYGPYSVT |
| | NCGEHDTTEYKLRTLQVSPLDNGDLIREIWHYQYLSWPDHGVPSEPGGVLSFLDQINQRQESL |
| | PHAGPIIVHCSAGIGRTGTIIVIDMLMENISTKGLDCDIDIQKTIQMVRAQRSGMVQTEAQYKFI |
| | YVAIAQF |
| | [BCD21 a.a. seq.] |
| 241 | MPPPRLLFFLLFLTPMEVRPEEPLVVKVEEGDNAVLQCLKGTSDGPTQQLTWSRESPLKPFLK |
| | LSLGLPGLGIHMRPLAIWLFIFNVSQQMGGFYLCQPGPPSEKAWQPGWTVNVEGSGELFRWN |
| | VSDLGGLGCGLKNRSSEGPSSPSGKLMSPKLYVWAKDRPEIWEGEPPCLPPRDSLNQSLSQDL |
| | TMAPGSTLWLSCGVPPDSVSRGPLSWTHVHPKGPKSLLSLELKDDRPARDMWVMETGLLLPR |
| | ATAQDAGKYYCHRGNLTMSFHLEITARPVLWHWLLRTGGWKVSAVTLAYLIFCLCSLVGILH |
| | LGGGGSGGGGSMSAIQAAWPSGTECIAKYNFHGTAEQDLPFCKGDVLTIVAVTKDPNAYKAK |
| | NKVGREGIIPANYVQKREGVKAGTKLSLMPWFHGKITREQAERLLYPPETGLFLVKESTNYPG |
| | DYTLCVSCDGKVEHYRIMYHASKLSIDEEVYFENLMQLVAHYTSDADGLCTRLIKPKVMEGT |
| | VAAQDEFYRSGWALNMKELKLLQTIGKGEFGDVMLGDYRGNKVAVKCIKNDATAQAFLAE |
| | ASVMTQLRHSNLVQLLGVIVEEKGGLYIVTEYMAKGSLVDYLRSRGRSVLGGDCLLKFSLDV |
| | CEAMEYLEGNNFVHRDLAARNVLVSEDNVAKVSDFGLTKEASSTQDTGKLPVKWTAPEALR |
| | EKKFSTKSDVWSFGILLWEIYSFGRVPYPRIPLKDVVPRVEKGYKMDAPDGCPPAVYLVMKN |
| | CWHLDAAMRPSFLQLREQLEHIKTHELH |
| | [BCD22 a.a. seq.] |
| 242 | MPGGPGVLQALPATIFLLFLLSAVYLGPGCQALWMHKVPASLMVSLGEDAHFQCPHNSSNNA |
| | NVTWWRVLHGNYTWPPEFLGPGEDPNGTLIIQNVNKSHGGIYVCRVQEGNESYQQSCGTYLR |
| | VRQPPPRPFLDMGEGTKNRIITAEGIILLFCAVVPGTLLLFRKRWQNEKLGLGGGGSGMSAIQA |
| | AWPSGTECIAKYNFHGTAEQDLPFCKGDVLTIVAVTKDPNAYKAKNKVGREGIIPANYVQKR |
| | EGVKAGTKLSLMPWFHGKITREQAERLLYPPETGLFLVKESTNYPGDYTLCVSCDGKVEHYRI |
| | MYHASKLSIDEEVYFENLMQLVAHYTSDADGLCTRLIKPKVMEGTVAAQDEFYRSGWALNM |
| | KELKLLQTIGKGEFGDVMLGDYRGNKVAVKCIKNDATAQAFLAEASVMTQLRHSNLVQLLG |
| | VIVEEKGGLYIVTEYMAKGSLVDYLRSRGRSVLGGDCLLKFSLDVCEAMEYLEGNNFVHRDL |
| | AARNVLVSEDNVAKVSDFGLTKEASSTQDTGKLPVKWTAPEALREKKFSTKSDVWSFGILLW |
| | EIYSFGRVPYPRIPLKDVVPRVEKGYKMDAPDGCPPAVYLVMKNCWHLDAAMRPSFLQLRE |
| | QLEHIKTHELH |
| | [BCD23 a.a. seq.] |
| 243 | MARLALSPVPSHWMVALLLLLSAEPVPAARSEDRYRNPKGSACSRIWQSPRFIARKRGFTVK |
| | MHCYMNSASGNVSWLWKQEMDENPQQLKLEKGRMEESQNESLATLTIQGIRFEDNGIYFCQ |
| | QKCNNTSEVYQGCGTELRVMGFSTLAQLKQRNTLKDGIIMIQTLLIILFIIVPIFLLLDKDGGGG |
| | SMSAIQAAWPSGTECIAKYNFHGTAEQDLPFCKGDVLTIVAVTKDPNAYKAKNKVGREGIIPA |
| | NYVQKREGVKAGTKLSLMPWFHGKITREQAERLLYPPETGLFLVKESTNYPGDYTLCVSCDG |
| | KVEHYRIMYHASKLSIDEEVYFENLMQLVAHYTSDADGLCTRLIKPKVMEGTVAAQDEFYRS |
| | GWALNMKELKLLQTIGKGEFGDVMLGDYRGNKVAVKCIKNDATAQAFLAEASVMTQLRHS |
| | NLVQLLGVIVEEKGGLYIVTEYMAKGSLVDYLRSRGRSVLGGDCLLKFSLDVCEAMEYLEGN |
| | NFVHRDLAARNVLVSEDNVAKVSDFGLTKEASSTQDTGKLPVKWTAPEALREKKFSTKSDV |
| | WSFGILLWEIYSFGRVPYPRIPLKDVVPRVEKGYKMDAPDGCPPAVYEVMKNCWHLDAAMR |
| | PSFLQLREQLEHIKTHELH |
| | [BCD24 a.a. seq.] |

What is claimed is:

1. A polynucleotide encoding a chimeric polypeptide that inhibits immune cell activity, wherein the polypeptide comprises a first domain that mediates association of the polypeptide with an immune cell component and a second domain that mediates inhibition of immune cell activity when the polypeptide is expressed in the immune cell.

2. The polynucleotide of claim 1, which is a messenger RNA (mRNA).

3. The polynucleotide of claim 2, which is a modified messenger RNA (mmRNA).

4. The polynucleotide of any one of claim 1-3, wherein immune cell activity is inhibited without depletion of the immune cell.

5. The polynucleotide of any one of claims 1-4, wherein the immune cell is a T cell.

6. The polynucleotide of claim 5, wherein the first domain is from a membrane-associated protein expressed in T cells.

7. The polynucleotide of claim 6, wherein the first domain is from Fyn, Src or KRAS.

8. The polynucleotide of claim 7, wherein first domain is an N-terminal membrane-binding portion of human Fyn.

9. The polynucleotide of claim 7, wherein the first domain is an N-terminal membrane-binding portion of human Src.

10. The polynucleotide of claim 7, wherein the first domain is an C-terminal membrane-binding portion of human KRAS.

11. The polynucleotide of claim 5, wherein the first domain is from a transmembrane-associated protein expressed in T cells.

12. The polynucleotide of claim 11, wherein the first domain is an N-terminal membrane-binding portion of human PAG.

13. The polynucleotide of claim 5, wherein the first domain is from a protein expressed in T cells that associates with a membrane receptor.

14. The polynucleotide of claim 13, wherein the first domain is from Lck or ZAP-70.

15. The polynucleotide of claim 14, wherein the first domain is a human Lck polypeptide comprising SH2 and SH3 domains.

16. The polynucleotide of claim 14, wherein the first domain is a human ZAP-70 polypeptide comprising at least one SH2 domain.

17. The polynucleotide of claim 5, wherein the first domain is from an intracellular protein expressed in T cells.

18. The polynucleotide of claim 17, wherein the first domain is from a protein selected from the group consisting of LAT, Grb2, Grap, PI3K.p85α, PLCγ1, GADS, ADAP, NCK, VAV, SOS, ITK and SLP76.

19. The polynucleotide of claim 18, wherein the first domain is a human LAT polypeptide selected from the group consisting of a full-length human LAT protein, an N-terminal portion of human LAT and a ZAP-70-binding portion of human LAT.

20. The polynucleotide of claim 18, wherein the first domain is a Grb2 polypeptide comprising an SH2 domain.

21. The polynucleotide of claim 18, wherein the first domain is a Grap polypeptide comprising an SH2 domain.

22. The polynucleotide of claim 18, wherein the first domain is a PI3K.p85α polypeptide in which an internal region containing an iSH2 domain has been deleted.

23. The polynucleotide of claim 18, wherein the first domain is a PLCγ1 polypeptide comprising SH2 and SH3 domains.

24. The polynucleotide of claim 5, wherein the second domain comprises an ITIM motif.

25. The polynucleotide of claim 24, wherein the second domain comprises a human LAIR1 ITIM1 motif.

26. The polynucleotide of claim 24, wherein the second domain comprises a human LAIR1 ITIM2 motif.

27. The polynucleotide of claim 24, wherein the second domain comprises a human CTLA4 ITIM-like motif.

28. The polynucleotide of claim 5, wherein the second domain comprises an inhibitory kinase domain.

29. The polynucleotide of claim 28, wherein the second domain comprises a constitutively active Csk polypeptide.

30. The polynucleotide of claim 29, wherein the second domain comprises a constitutively active human Csk polypeptide comprising W47A, R107K and E14A mutations.

31. The polynucleotide of claim 5, wherein the second domain comprises a phosphatase domain.

32. The polynucleotide of claim 31, wherein the second domain comprises a SHP1 polypeptide having phosphatase activity.

33. The polynucleotide of claim 31, wherein the second domain comprises a SHIP1 polypeptide having phosphatase activity.

34. The polynucleotide of claim 31, wherein the second domain comprises a PTPN22 polypeptide having phosphatase activity.

35. The polynucleotide of claim 31, wherein the second domain comprises a PTPN1 polypeptide having phosphatase activity.

36. The polynucleotide of claim 5, wherein the second domain inhibits PI3K activity in the T cell.

37. The polynucleotide of claim 36, wherein the second domain is from a human PTEN protein.

38. The polynucleotide of claim 5, wherein the first domain has an amino acid sequence selected from the group consisting of SEQ ID NOs: 1-20.

39. The polynucleotide of claim 5, wherein the second domain has an amino acid sequence selected from the group consisting of SEQ ID NOs: 21-34.

40. The polynucleotide of claim 5, which encodes a chimeric polypeptide comprising a first domain from a human LAT protein and a second domain comprising a LAIR1 or CTLA4 ITIM motif.

41. The polynucleotide of claim 5, which encodes a chimeric polypeptide comprising a first domain from a human protein selected the group consisting of LAT, PAG, Lck, Fyn and Src and a second domain comprising a constitutively active human CSK protein.

42. The polynucleotide of claim 5, which encodes a chimeric polypeptide comprising a first domain from a human protein selected the group consisting of LAT, Src, PI3K.p85 and PLCγ1 and a second domain from a human protein selected from the group consisting of SHP1, SHIP1 and PTPN22.

43. The polynucleotide of claim 5, which encodes a chimeric polypeptide comprising a first domain from a human PLCγ1 protein and a second domain from a human PTEN protein.

44. The polynucleotide of claim 5, which comprises a nucleotide sequence shown in any one of SEQ ID NOs: 35-80.

45. The polynucleotide of claim 5, which encodes a chimeric polypeptide comprising an amino acid sequence shown in any one of SEQ ID NOs: 81-126.

46. The polynucleotide of any one of claims 5-45, which inhibits T cell proliferation when expressed in the T cell.

47. The polynucleotide of any one of claims 5-45, which inhibits T cell cytokine production when expressed in the T cell.

48. The polynucleotide of any one of claims 1-4, wherein the immune cell is a B cell.

49. The polynucleotide of claim 48, wherein the first domain is from a membrane associated protein expressed in B cells.

50. The polynucleotide of claim 49, wherein the first domain is from CD79a, CD79b or Syk.

51. The polynucleotide of claim 50, wherein the first domain is a human CD79a polypeptide that lacks ITAMs or has inactivated ITAMs.

52. The polynucleotide of claim 50, wherein the first domain is a human CD79b polypeptide that lacks ITAMs or has inactivated ITAMs.

53. The polynucleotide of claim 48, wherein the first domain is from a membrane receptor expressed in B cells.

54. The polynucleotide of claim 53, wherein the first domain is from CD19 or CD64.

55. The polynucleotide of claim 54, wherein the first domain is a human CD19 polypeptide that lacks ITAMs or has inactivated ITAMs.

56. The polynucleotide of claim 54, wherein the first domain is an N-terminal portion of human CD64.

57. The polynucleotide of claim 48, wherein the second domain alters CD19/CD22 balance in the B cell.

58. The polynucleotide of claim 48, wherein the second domain is from CD22 or SHP1.

59. The polynucleotide of claim 58, wherein the second domain comprises a human CD22 ITIM motif.

60. The polynucleotide of claim 58, wherein the second domain comprises a human SHP1phosphatase domain.

61. The polynucleotide of claim 48, wherein the second domain inhibits B Cell Receptor (BCR) activity in the B cell.

62. The polynucleotide of claim 61, wherein the second domain comprises a CD22 ITIM motif.

63. The polynucleotide of claim 48, wherein the second domain alters FcR activity in the B cell.

64. The polynucleotide of claim 63, wherein the second domain is from CD32b.

65. The polynucleotide of claim 64, wherein the second domain comprises a human CD32b ITIM motif.

66. The polynucleotide of claim 48, wherein the first domain has an amino acid sequence selected from the group consisting of SEQ ID NOs: 127-143 and 229-231.

67. The polynucleotide of claim 48, wherein the second domain has an amino acid sequence selected from the group consisting of SEQ ID NOs: 25, 26 and 144-149.

68. The polynucleotide of claim 48, wherein the first domain is from a human protein selected from the group consisting of CD79a, CD79b, CD19 and Syk and the second domain is from human CD22, human SHP1 or human Csk.

69. The polynucleotide of claim 48, wherein the first domain is from human CD64 and the second domain is from human CD32b.

70. The polynucleotide of claim 48, which comprises a nucleotide sequence shown in any one of SEQ ID NOs: 150-167 or 232-237.

71. The polynucleotide of claim 48, which encodes a chimeric polypeptide comprising an amino acid sequence shown in any one of SEQ ID NOs: 168-185 or 238-243.

72. The polynucleotide of any one of claims 48-71, which inhibits B cell immunoglobulin production when expressed in the B cell.

73. The polynucleotide of any one of claims 48-71, which inhibits B cell cytokine production when expressed in the B cell.

74. The polynucleotide of any one of claims 1-4, wherein the immune cell is an NK cell.

75. The polynucleotide of any one of claims 1-4, wherein the immune cell is a dendritic cell.

76. The polynucleotide of any one of claims 1-4, wherein the immune cell is a macrophage.

77. A lipid nanoparticle comprising the polynucleotide of any one of claims 1-76.

78. The lipid nanoparticle of claim 77, which comprises an immune cell delivery potentiating lipid.

79. A pharmaceutical composition comprising the lipid nanoparticle of claim 77 or claim 78 and a pharmaceutically acceptable carrier.

80. Use of a lipid nanoparticle of claim 77 or claim 78, and an optional pharmaceutically acceptable carrier, in the manufacture of a medicament for inhibiting an immune response in an individual, wherein the medicament comprises the lipid nanoparticle and an optional pharmaceutically acceptable carrier and wherein the treatment comprises administration of the medicament, and an optional pharmaceutically acceptable carrier.

81. A kit comprising a container comprising the lipid nanoparticle of claim 77 or claim 78, and an optional pharmaceutically acceptable carrier, and a package insert comprising instructions for administration of the lipid nanoparticle for inhibiting an immune response in an individual.

82. A method of inhibiting an immune response in a subject, the method comprising administering the lipid nanoparticle of claim 77 or claim 78, and an optional pharmaceutically acceptable carrier, to the subject such that an immune response is inhibited in the subject.

83. A method of inhibiting a T cell response in a subject, the method comprising administering to the subject the polynucleotide of any one of claims 5-47, wherein the polynucleotide is encapsulated in a lipid nanoparticle comprising an immune cell delivery potentiating lipid, such that a T cell response is inhibited in the subject.

84. A method of inhibiting a B cell response in a subject, the method comprising administering to the subject the polynucleotide of any one of claims 48-73, wherein the polynucleotide is encapsulated in a lipid nanoparticle comprising an immune cell delivery potentiating lipid, such that a B cell response is inhibited in the subject.

85. The method of any one of claims 82-84, wherein the subject has an autoimmune disease.

86. The method of claim 85, wherein the autoimmune disease is selected from the group consisting of rheumatoid arthritis, systemic lupus erythematosus, inflammatory bowel disease (including ulcerative colitis and Crohn's disease), Type 1 diabetes, multiple sclerosis, psoriasis, Graves' disease, Hashimoto's thyroiditis, chronic inflammatory demyelinating polyneuropathy, Guillain-Barre syndrome, myasthenia gravis, glomerulonephritis and vasculitis.

87. The method of any one of claims 82-84, wherein the subject has an allergic disorder.

88. The method of any one of claims 82-84, wherein the subject has an inflammatory reaction.

89. The method of any one of claims 82-84, wherein the subject is a transplant recipient.

90. The method of any one of claims 82-84, wherein the subject is undergoing immunotherapy.

91. An immune cell delivery LNP comprising:
(i) an ionizable lipid;
(ii) a sterol or other structural lipid;
(iii) a polynucleotide of any one of claims 1-76;
(iv) optionally, a non-cationic helper lipid or phospholipid; and
(v) optionally, a PEG-lipid;
wherein one or more of (i) the ionizable lipid or (ii) the sterol or other structural lipid comprises an immune cell delivery potentiating lipid in an amount effective to enhance delivery of the LNP to a target immune cell, wherein the target immune cell is a T cell or a B cell.

92. The immune cell delivery LNP of claim 91, which comprises a phytosterol or a combination of a phytosterol and cholesterol.

93. The immune cell delivery LNP of claim 92, wherein the phytosterol is selected from the group consisting of β-sitosterol, stigmasterol, β-sitostanol, campesterol, brassicasterol, and combinations thereof.

94. The immune cell delivery LNP of claim 92, wherein the phytosterol comprises a sitosterol or a salt or an ester thereof.

95. The immune cell delivery LNP of claim 92, wherein the phytosterol comprises a stigmasterol or a salt or an ester thereof.

96. The immune cell delivery LNP of claim 92, wherein the phytosterol is beta-sitosterol 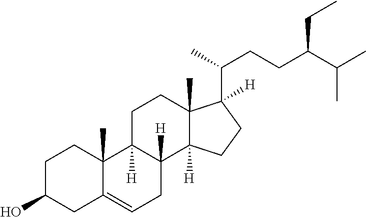
or a salt or an ester thereof.

97. The immune cell delivery lipid LNP of claim 91, wherein the phytosterol or a salt or ester thereof is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

98. The immune cell delivery LNP of claim 97, wherein the phytosterol is β-sitosterol.

99. The immune cell delivery LNP of claim 97, wherein the phytosterol is β-sitostanol.

100. The immune cell delivery LNP of claim 97, wherein the phytosterol is campesterol.

101. The immune cell delivery LNP of claim 97, wherein the phytosterol is brassicasterol.

102. The immune cell delivery LNP of any one of claims 91-101, wherein the ionizable lipid comprises a compound of any of Formulae (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8).

103. The immune cell delivery LNP of any one of claims 91-101, wherein the ionizable lipid comprises a compound selected from the group consisting of Compound X, Compound Y, Compound I-48, Compound I-50, Compound I-109, Compound I-111, Compound I-113, Compound I-181, Compound I-182, Compound I-244, Compound I-292, Compound I-301, Compound I-309, Compound I-317, Compound I-321, Compound I-322, Compound I-326, Compound I-328, Compound I-330, Compound I-331, Compound I-332, Compound I-347, Compound I-348, Compound I-349, Compound I-350, Compound I-352 and Compound I-M.

104. The immune cell delivery LNP of any one of claims 91-101, wherein the ionizable lipid comprises a compound selected from the group consisting of Compound X, Compound Y, Compound I-321, Compound I-292, Compound I-326, Compound I-182, Compound I-301, Compound I-48, Compound I-50, Compound I-328, Compound I-330, Compound I-109, Compound I-111 and Compound I-181.

105. The immune cell delivery LNP of any one of claims 91-104, wherein the LNP comprises a phospholipid, and wherein the phospholipid comprises a compound selected from the group consisting of DSPC, DMPE, and Compound H-409.

106. The immune cell delivery LNP of any one of claims 91-105, wherein the LNP comprises a PEG-lipid.

107. The immune cell delivery LNP of claim 106, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

108. The immune cell delivery LNP of claim 107, wherein the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25.

109. The immune cell delivery LNP of claim 108, wherein the PEG lipid comprises a compound selected from the group consisting of Compound P-428, Compound PL-16, Compound PL-17, Compound PL-18, Compound PL-19, Compound PL-1, and Compound PL-2.

110. The immune cell delivery LNP of any one of claims 91-109, which comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid.

111. The immune cell delivery LNP of any one of claims 91-109, which comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid.

112. The immune cell delivery LNP of any one of claims 91-109, which comprises about 50 mol % ionizable lipid, about 10 mol % non-cationic helper lipid or phospholipid, about 38.5 mol % sterol or other structural lipid, and about 1.5 mol % PEG lipid.

113. The immune cell delivery LNP of any one of claims 109-112, wherein the mol % sterol or other structural lipid is 18.5% phytosterol and the total mol % structural lipid is 38.5%.

114. The immune cell delivery LNP of any one of claims 109-112, wherein the mol % sterol or other structural lipid is 28.5% phytosterol and the total mol % structural lipid is 38.5%.

115. The immune cell delivery LNP of any one of claims 91-109, which comprises:
(i) about 50 mol % ionizable lipid, wherein the ionizable lipid is a compound selected from the group consisting of Compound I-301, Compound I-321, and Compound I-326;
(ii) about 10 mol % phospholipid, wherein the phospholipid is DSPC;
(iii) about 38.5 mol % structural lipid, wherein the structural lipid is selected from β-sitosterol and cholesterol; and
(iv) about 1.5 mol % PEG lipid, wherein the PEG lipid is Compound P-428.


SEQUENCE LISTING (245 sequences)

SEQ ID NO: 1 - protein, 337 aa, Artificial Sequence
  Synthetic hs.ZAP70(nSH2-IA-cSH2-IB)
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNL

SEQ ID NO: 2 - protein, 337 aa, Artificial Sequence
  Synthetic hs.ZAP70(nSH2-IA-cSH2-IB.Y/A)
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGATPEPARITSPDKPRPMPMDTSVAESPASDPEELKDKKLFLKRDNL

SEQ ID NO: 3 - protein, 254 aa, Artificial Sequence
  Synthetic hs.ZAP70(nSH2-IA-cSH2-GS4)
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEAC

SEQ ID NO: 4 - protein, 214 aa, Artificial Sequence
  Synthetic hs.ZAP70(nSH2-GS-cSH2-GS4)
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCGGGGSGGGGSGGGGSGGGGSWYHSSLTREEAERKLYSGAQTDGKFLLRPRKEQGTYALSLIYGKTVYHYLISQDKAGKYCIPEGTKFDTLWQLVEYLKLKADGLIYCLKEAC

SEQ ID NO: 5 - protein, 93 aa, Artificial Sequence
  Synthetic hs.Grb2(SH2)
WFFGKIPRAKAEEMLSKQRHDGAFLIRESESAPGDFSLSVKFGNDVQHFKVLRDGAGKYFLWVVKFNSLNELVDYHRSTSVSRNQQIFLRDIE

SEQ ID NO: 6 - protein, 96 aa, Artificial Sequence
  Synthetic hs.Grap(SH2)
MWYSGRISRQLAEEILMKRNHLGAFLIRESESSPGEFSVSVNYGDQVQHFKVLREASGKYFLWEEKFNSLNELVDFYRTTTIAKKRQIFLRDEEPL

SEQ ID NO: 7 - protein, 244 aa, Artificial Sequence
  Synthetic hs.Lck(SH2-SH3)
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRET

SEQ ID NO: 8 - protein, 262 aa, Artificial Sequence
  Synthetic hs.LAT
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGALVVLPDSTPATSTAAPSAPALSTPGIRDSAFSMESIDDAVNVPESGESAEASLDGSREAVNVSQELHPGAAKTEPAALSSQEAEEVEEEGAPDYENLQELN

SEQ ID NO: 9 - protein, 160 aa, Artificial Sequence
  Synthetic hs.LAT(1-160)
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPG

SEQ ID NO: 10 - protein, 38 aa, Artificial Sequence
  Synthetic hs.LAT(1-38)
MEEAILVPCVLGLLLLPILAMLMALCVHCHRLPGSYDS

SEQ ID NO: 11 - protein, 33 aa, Artificial Sequence
  Synthetic hs.LAT(1-33)
MEEAILVPCVLGLLLLPILAMLMALCVHCHRLP

SEQ ID NO: 12 - protein, 47 aa, Artificial Sequence
  Synthetic hs.PAG(1-47)
MGPAGSLLGSGQMQITLWGSLAAVAIFFVITFLIFLCSSCDREKKPR

SEQ ID NO: 13 - protein, 50 aa, Artificial Sequence
  Synthetic hs.Lck(1-50)
MGCGCSSHPEDDWMENIDVCENCHYPIVPLDGKGTLLIRNGSEVRDPLVT

SEQ ID NO: 14 - protein, 50 aa, Artificial Sequence
  Synthetic hs.Fyn(1-50)
MGCVQCKDKEATKLTEERDGSLNQSSGYRYGTDPTPQHYPSFGVTSIPNY

SEQ ID NO: 15 - protein, 10 aa, Artificial Sequence
  Synthetic hs.Src(1-10)
MGSNKSKPKD

SEQ ID NO: 16 - protein, 38 aa, Artificial Sequence
  Synthetic mm.LAT(1-38)
MEADALSPVGLGLLLLPFLVTLLAALCVRCRELPVSYD

SEQ ID NO: 17 - protein, 533 aa, Artificial Sequence
  Synthetic hs.PI3K.p85 (del.iSH2)
MSAEGYQYRALYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQPAPALPPKPPKPTTVANNGMNNNMSLQDAEWYWGDISREEVNEKLRDTADGTFLVRDASTKMHGDYTLTLRKGGNNKLIKIFHRDGKYGFSDPLTFSSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 18 - protein, 302 aa, Artificial Sequence
  Synthetic hs.PLCg1(SH2/3)
WFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVN

SEQ ID NO: 19 - protein, 21 aa, Artificial Sequence
  Synthetic KRAS
YRLKKISKEEKTPGCVKIKKC

SEQ ID NO: 20 - protein, 72 aa, Artificial Sequence
  Synthetic Lck(1-72)
MGCGCSSHPEDDWMENIDVCENCHYPIVPLDGKGTLLIRNGSEVRDPLVTYEGSNPPASPLQDNLVIALHSY

SEQ ID NO: 21 - protein, 272 aa, Artificial Sequence
  Synthetic SHP1(PTP)
FWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 22 - protein, 108 aa, Artificial Sequence
  Synthetic (Y/A/LAIR1.ITIM1)
VTYAQLLPDSTPATSTAAPSAPALSTPGIRDSAFSMESIDDVTYAQLPESGESAEASLDGSREVTYAQLSQELHPGAAKTEPAALSSQEAEEVEEEGAPDYENLQELN

SEQ ID NO: 23 - protein, 108 aa, Artificial Sequence
  Synthetic (Y/A/LAIR1.ITIM2)
ITYAAVLPDSTPATSTAAPSAPALSTPGIRDSAFSMESIDDITYAAVPESGESAEASLDGSREITYAAVSQELHPGAAKTEPAALSSQEAEEVEEEGAPDYENLQELN

SEQ ID NO: 24 - protein, 102 aa, Artificial Sequence
  Synthetic LAIR1(187-287/ITIM)
NHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 25 - protein, 450 aa, Artificial Sequence
  Synthetic CA.Csk (W47A/R107K/E154A)
MSAIQAAWPSGTECIAKYNFHGTAEQDLPFCKGDVLTIVAVTKDPNAYKAKNKVGREGIIPANYVQKREGVKAGTKLSLMPWFHGKITREQAERLLYPPETGLFLVKESTNYPGDYTLCVSCDGKVEHYRIMYHASKLSIDEEVYFENLMQLVAHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 26 - protein, 255 aa, Artificial Sequence
  Synthetic Csk(195-449)
LKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELH

SEQ ID NO: 27 - protein, 514 aa, Artificial Sequence
  Synthetic SHP1(2-515)
VRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 28 - protein, 41 aa, Artificial Sequence
  Synthetic CTLA4(ITIM)
AVSLSKMLKKRSPLTTGVYVKMPPTEPECEKQFQPYFIPIN

SEQ ID NO: 29 - protein, 287 aa, Artificial Sequence
  Synthetic PTPN1(3-277)
GFGGGGSMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGGKPST

SEQ ID NO: 30 - protein, 349 aa, Artificial Sequence
  Synthetic PTEN(1-350) (K13E/K289E)
TAIIKEIVSRNERRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEEVENGSLCDQEIDSICSIERADNDKEYLVLTLTKNDLDKANKDKANRYFSPNFKVKLYFTKT

SEQ ID NO: 31 - protein, 800 aa, Artificial Sequence
  Synthetic SHIP1(111-910)
DPEEDTVESVVSPPELPPRNIPLTASSCEAKEVPFSNENPRATETSRPSLSETLFQRLQSMDTSGLPEEHLKAIQDYLSTQLAQDSEFVKTGSSSLPHLKKLTTLLCKELYGEVIRTLPSLESLQRLFDQQLSPGLRPRPQVPGEANPINMVSKLSQLTSLLSSIEDKVKALLHEGPESPHRPSLIPPVTFEVKAESLGIPQKMQLKVDVESGKLIIKKSKDGSEDKFYSHKKILQLIKSQKFLNKLVILVETEKEKILRKEYVFADSKKREGFCQLLQQMKNKHSEQPEPDMITIFIGTWNMGNAPPPKKITSWFLSKGQGKTRDDSADYIPHDIYVIGTQEDPLSEKEWLEILKHSLQEITSVTFKTVAIHTLWNIRIVVLAKPEHENRISHICTDNVKTGIANTLGNKGAVGVSFMFNGTSLGFVNSHLTSGSEKKLRRNQNYMNILRFLALGDKKLSPFNITHRFTHLFWFGDLNYRVDLPTWEAETIIQKIKQQQYADLLSHDQLLTERREQKVFLHFEEEEITFAPTYRFERLTRDKYAYTKQKATGMKYNLPSWCDRVLWKSYPLVHVVCQSYGSTSDIMTSDHSPVFATFEAGVTSQFVSKNGPGTVDSQGQIEFLRCYATLKTKSQTKFYLEFHSSCLESFVKSQEGENEEGSEGELVVKFGETLPKLKPIISDPEYLLDQHILISIKSSDSDESYGEGCIALRLEATETQLPIYTPLTHHGELTGHFQGEIKLQTSQGKTREKLYDFVKTERDESSGPKTLKSLTSHDPMKQWEVTSRAPPCSGSSITEI

SEQ ID NO: 32 - protein, 290 aa, Artificial Sequence
  Synthetic h.s. PTPN22(N290)
MDQREILQKFLDEAQSKKITKEEFANEFLKLKRQSTKYKADKTYPTTVAEKPKNIKKNRYKDILPYDYSRVELSLITSDEDSSYINANFIKGVYGPKAYIATQGPLSTTLLDFWRMIWEYSVLIIVMACMEYEMGKKKCERYWAEPGEMQLEFGPFSVSCEAEKRKSDYIIRTLKVKFNSETRTIYQFHYKNWPDHDVPSSIDPILELIWDVRCYQEDDSVPICIHCSAGCGRTGVICAIDYTWMLLKDGIIPENFSVFSLIREMRTQRPSLVQTQEQYELVYNAVLELF

SEQ ID NO: 33 - protein, 290 aa, Artificial Sequence
  Synthetic hs.PTPN22(1-290; S35A)
MDQREILQKFLDEAQSKKITKEEFANEFLKLKRQATKYKADKTYPTTVAEKPKNIKKNRYKDILPYDYSRVELSLITSDEDSSYINANFIKGVYGPKAYIATQGPLSTTLLDFWRMIWEYSVLIIVMACMEYEMGKKKCERYWAEPGEMQLEFGPFSVSCEAEKRKSDYIIRTLKVKFNSETRTIYQFHYKNWPDHDVPSSIDPILELIWDVRCYQEDDSVPICIHCSAGCGRTGVICAIDYTWMLLKDGIIPENFSVFSLIREMRTQRPSLVQTQEQYELVYNAVLELF

SEQ ID NO: 34 - protein, 266 aa, Artificial Sequence
  Synthetic hs. PTPN22 (24-289; S35A)
FANEFLKLKRQATKYKADKTYPTTVAEKPKNIKKNRYKDILPYDYSRVELSLITSDEDSSYINANFIKGVYGPKAYIATQGPLSTTLLDFWRMIWEYSVLIIVMACMEYEMGKKKCERYWAEPGEMQLEFGPFSVSCEAEKRKSDYIIRTLKVKFNSETRTIYQFHYKNWPDHDVPSSIDPILELIWDVRCYQEDDSVPICIHCSAGCGRTGVICAIDYTWMLLKDGIIPENFSVFSLIREMRTQRPSLVQTQEQYELVYNAVLEL

SEQ ID NO: 35 - RNA, 1827 nt, Artificial Sequence
  Synthetic TCD1 nt. seq.
augccugacccugccgcccaccugccuuucuucuacggcagcaucagcagagccgaggccgaggagcaccugaagcuggccggcauggccgacggccuguuccugcugagacagugccugagaagccugggcggcuacgugcugagccuggugcacgacgugagauuccaccacuucccuaucgagagacagcugaacggcaccuacgccaucgccggcggcaaggcccacugcggcccugccgagcugugcgaguucuacagcagagauccugacggcuugccuugcaaccugagaaagccguguaauagaccuagcggccuggagccucagccuggcguguucgacugucugcgcgacgccauggugagagacuacgugagacagaccuggaagcuggagggcgaggcccuggagcaggccaucaucagccaggccccucagguggagaagcugaucgccaccaccgcccacgagagaaugccuugguaccacagcagccugaccagagaggaagccgagagaaagcuguacagcggcgcccagaccgacggcaaguucuugcuucggccgagaaaggagcagggcacuuacgcgcucaguuugaucuacggaaagaccguguaccacuaccugauuucucaggacaaggccggcaaguacugcaucccugagggcaccaaguucgacacccuguggcagcugguggaguaucugaagcuuaaggcugacggacugaucuacugccugaaggaggccugcccuaacagcagcgccagcaacgccaguggugcagccgccccuacccugccugcccacccuagcacccugacccacccucagagaagaauugacacacugaacagcgacggcuacacaccagagccugccagaaucaccagcccugacaagccuagaccuaugccuauggacaccagcguguacgagagcccuuacagcgacccugaggaguuaaaggacaagaaguuauucuugaagagagacaaccuguucugggaggaguucgagucgcugcagaagcaggaggugaagaaccugcaccagagacuggagggacaacggccagagaacaagggcaagaacagauacaagaacaucuugccauucgaucacagccgggugauccugcagggcagggauuccaacaucccuggcagcgacuacaucaacgccaauuacauuaagaaccagcugcugggcccugacgagaacgccaagaccuacaucgccagccagggcugccuggaggccaccgugaacgacuucuggcagauggccuggcaggagaauucuagggugaucgugaugaccacccgcgaaguggagaagggcagaaacaagugcgugccuuacuggccugaggugggcaugcagagagccuacggcccauacagcgugaccaacugcggcgagcacgacaccaccgaguacaagcugagaacccugcaggugagcccuuuggauaacggcgaccugaucagagagaucuggcacuaccaguaccugucuuggccugaucacggcgugccuagcgaaccaggcggcgugcuauccuuccuggaccagaucaaccaacgacaggagucccuaccucacgccggcccuaucaucgugcacugcagcgccggcaucggcagaaccggcaccauuauugugaucgacaugcucauggagaacaucagcaccaagggccuggacugcgacaucgacauccagaagaccauccagauggugcgggcucagcgcagcggcauggugcagaccgaggcccaguacaaguucaucuacguggccaucgcacaguuc

SEQ ID NO: 36 - RNA, 1827 nt, Artificial Sequence
  Synthetic TCD2 nt. seq.
augccugacccugccgcccaccugccuuucuucuacggcagcaucagcagagccgaggccgaggagcaccugaagcuggccggcauggccgacggccuguuccugcugagacagugccugagaagccugggcggcuacgugcugagccuggugcacgacgugagauuccaccacuucccuaucgagagacagcugaacggcaccuacgccaucgccggcggcaaggcccacugcggcccugccgagcugugcgaguucuacagccgggacccugacggccucccuugcaaccugagaaagccuuguaacagaccuagcggccuggagccucagccuggcguguucgacugucugcgcgacgccauggugagagacuacgugagacagaccuggaagcuggagggcgaggcccuggagcaggccaucaucagccaggccccucagguggagaagcugaucgccaccaccgcccacgagagaaugccuugguaccacagcagccugaccagagaggaagccgaaagaaagcuguacagcggcgcccagaccgacggcaaguucuugcugaggccuagaaaggagcaggguacauacgcacuuucccugauuuacggcaagaccguguaccacuaccugauuucccaagacaaggccggcaaguacugcaucccugagggcaccaaguucgacacccuguggcagcugguggaguauuuaaagcucaaggccgacggccuaaucuauugucucaaggaggccugcccuaacagcagcgccagcaacgcuagcggagccgccgcaccuacccugccugcccacccuagcacccugacccacccucagagaagaaucgauacuuugaacagcgacggcgccacgccagaaccagccagaaucaccagcccugacaagccuagaccuaugccuauggacaccagcgucgcggaaagcccugccagcgacccugaggaacucaaggacaagaaguuauuccuaaagagagacaaccuguucugggaggaguucgagagccugcagaagcaggaggugaagaaccugcaccagagacuugagggucagaggccugagaacaagggcaagaacagauacaagaacauucugccauucgaucacucuagggugauccugcagggcagagacucuaacaucccuggcagcgacuacaucaacgccaacuauauaaagaaccaacugcugggcccugacgagaacgccaagaccuacaucgccagccagggcugccuggaggccaccgugaacgacuucuggcagauggccuggcaggagaauagccgagucauugugaugacuaccagagaagucgagaagggcagaaacaagugcgugccuuacuggccugaggugggcaugcagagagccuacggcccuuacagcgugacgaauugcggcgagcacgacaccaccgaguacaagcugagaacccugcaggugagcccucuggacaacggcgaucuuaucagagagaucuggcacuaccaguaucuguccuggccagaucacggcgugccuagcgagccaggcggcguuuugucauuccuggaccagaucaaucagcgacaggagagcuugccucacgccggcccuaucaucgugcacugcagcgccggcaucggcagaaccggcaccauuauugugaucgacaugcugauggagaacaucagcaccaagggccuggacugcgacaucgacauccagaagaccauccagauggugcgagcucagcgcagcggcauggugcagaccgaggcccaguacaaguucaucuacguggcuauugcgcaguuc

SEQ ID NO: 37 - RNA, 1608 nt, Artificial Sequence
  Synthetic TCD3 nt. seq.
augccugacccugccgcccaccugccuuucuucuacggcagcaucagcagagccgaggccgaggagcaccugaagcuggccggcauggccgacggccuguuccugcugagacagugccugagaagccugggcggcuacgugcugagccuggugcacgacgugagauuccaccacuucccuaucgagagacagcugaacggcaccuacgccaucgccggcggcaaggcccacugcggcccugccgagcugugcgaguucuacagccgggacccugacggacuuccuugcaaccugagaaagccuuguaauagaccuagcggccuggagccucagccuggcguguucgacuguuuacgagacgccauggugagagacuacgugagacagaccuggaagcuggagggcgaggcccuggagcaggccaucaucagccaggccccucagguggagaagcugaucgccaccaccgcccacgagagaaugccuugguaccacagcagccugaccagagaggaagccgagagaaagcuguacagcggcgcccagaccgacggcaaguuccuccugcggccuagaaaggagcagggaacauacgcguugucacuaaucuacggcaagaccguguaccacuaccugauuucccaggacaaggccggcaaguacugcaucccugagggcaccaaguucgacacccuguggcagcugguggaguauuugaagcugaaggcagacggauugaucuacugccucaaggaggccugcggcggaggaggaucaggaggcgguggcaguuucugggaggaguucgagagccugcagaagcaggaggugaagaaccugcaccagagauuggagggacagaggccagagaacaagggcaagaacagauacaagaacauucuuccuuucgaucacagucgggugauccugcagggcagagacucuaacaucccuggcagcgacuacaucaacgccaauuacauuaagaaccagcugcugggcccugacgagaacgccaagaccuacaucgccagccagggcugccuggaggccaccgugaacgacuucuggcagauggccuggcaggagaacucaagaguaauugugaugaccacaagggaagucgagaagggcagaaacaagugcgugccuuacuggccugaggugggcaugcagagagccuacggcccuuacagcgugaccaacugcggcgagcacgacaccaccgaguacaagcugagaacccugcaggugagcccucuggacaacggcgacuugauuagagagaucuggcacuaccaguaucugaguuggccagaccacggcgugccuagcgaaccugguggcguccuuaguuuccuggaccagaucaaccagaggcaggagucccugccucacgccggcccuaucaucgugcacugcagcgccggcaucggcagaaccggcaccauuauugugaucgacaugcugauggagaacaucagcaccaagggccuggacugcgacaucgacauccagaagaccauccagaugguuagggcccagagaagcggcauggugcagaccgaggcccaguacaaguucaucuacguggcaaucgcccaguuc

SEQ ID NO: 38 - RNA, 1488 nt, Artificial Sequence
  Synthetic TCD4 nt. seq.
augccugacccugccgcccaccugccuuucuucuacggcagcaucagcagagccgaggccgaggagcaccugaagcuggccggcauggccgacggccuguuccugcugagacagugccugagaagccugggcggcuacgugcugagccuggugcacgacgugagauuccaccacuucccuaucgagagacagcugaacggcaccuacgccaucgccggcggcaaggcccacugcggcccugccgagcugugcgaguucuacagccgugauccugacggacugccuugcaaccugagaaagccuugcggcggcggagguagcggcggugguggcaguggaggcggaggaucgggaggaggcggcuccugguaccacagcagccugaccagagaggaggccgaaagaaagcuguacagcggcgcccagaccgacggcaaguuccuguugcgcccuagaaaggagcagggcacuuacgcucugucguuaaucuacggcaagaccguguaccacuaccugaucagccaggacaaggccggcaaguacugcaucccugagggcaccaaguucgacacccuguggcagcucguggaguaucuaaagcucaaggccgacggccucaucuacugccugaaggaggccuguggagguggcggaagcggagguggugguuccuucugggaggaguucgagagccugcagaagcaggaggugaagaaccugcaccagagacuggagggccagcgaccggagaacaagggcaagaacagauacaagaacauucugccguucgaccacucacgcgugauccugcagggccgagauagcaacaucccuggcagcgacuacaucaacgccaauuauaucaagaaccagcugcugggcccugacgagaacgccaagaccuacaucgccagccagggcugccuggaggccaccgugaacgacuucuggcagauggccuggcaggagaauucucgagucauagugaugacuacccgggagguggagaagggcagaaacaagugcgugccuuacuggccugaggugggcaugcagagagccuacggcccuuacagcgugaccaacugcggcgagcacgacaccaccgaguacaagcugagaacccugcaggugagcccucuggacaacggcgaucugaucagagagaucuggcacuaccaauacuugucuuggccugaucacggcgugccuagcgagccuggcggcguauugucuuuccuggaccagaucaaucagagacaggagucccucccucacgccggcccuaucaucgugcacugcagcgccggcaucggcagaaccggcaccauaauugugaucgacaugcugauggagaacaucagcaccaagggccuggacugcgacaucgacauccagaagaccauccagauggugagagcccagagaagcggcauggugcagaccgaggcccaguacaaguucaucuacguggccauagcccaguuc

SEQ ID NO: 39 - RNA, 1140 nt, Artificial Sequence
  Synthetic TCD5 nt. seq.
ugguucuucggcaagaucccuagagccaaggccgaggagaugcugagcaagcagagacacgacggcgccuuccugaucagagagagcgagagcgccccuggcgacuucagccugagcgugaaguucggcaacgacgugcagcacuucaaggugcugagagacggcgccggcaaguacuuccuguggguggugaaguucaacagccugaacgagcugguggacuaccacagaagcaccagcgugagcagaaaccagcagaucuuccugagagacauugagggcggcggaggcagcggaggaggcggauccggaggaggaggcuccuucugggaggaguucgagagccugcagaagcaggaggugaagaaccugcaccagagacuggagggccaaagaccugagaacaagggcaagaacagauacaagaacauccugccuuucgaccacagcagagugauccugcagggcagagacagcaacaucccuggcagcgacuacaucaacgccaacuacaucaagaaccagcugcugggcccugacgagaacgccaagaccuacaucgccagccagggcugccuggaggccaccgugaacgacuucuggcagauggccuggcaggagaauucacgggucauugugaugaccaccagagagguggagaagggcagaaacaagugcgugccuuacuggccugaggugggcaugcagagagccuacggcccuuacagcgugaccaacugcggcgagcacgacaccaccgaguacaagcugagaacccugcaggugagcccucuggacaacggcgaccucauacgcgagaucuggcacuaccaguaccugagcuggccugaccacggcgugccuagcgagccuggcggcgugcugagcuuccuggaccagaucaaccagagacaggagagccugccucacgccggcccuaucaucgugcacugcagcgccggcaucggcagaaccggcaccaucaucgugaucgacaugcugauggagaacaucagcaccaagggccuggacugcgacaucgacauccagaagaccauccagauggugagagcccagagaagcggcauggugcagaccgaggcccaguacaaguucaucuacguggccaucgcccaguuc

SEQ ID NO: 40 - RNA, 1149 nt, Artificial Sequence
  Synthetic TCD6 nt. seq.
augugguacagcggcagaaucagcagacagcuggccgaggagauccugaugaagagaaaccaccugggcgccuuccugaucagagagagcgagagcagcccuggcgaguucagcgugagcgugaacuacggcgaccaggugcagcacuucaaggugcugagagaggccagcggcaaguacuuccugugggaggagaaguucaacagccugaacgagcugguggacuucuacagaaccaccaccaucgccaagaagagacagaucuuccugagagacgaggagccuuuaggaggaggcggaucuggugguggaggcaguggcggaggcgguucguucugggaggaguucgagagccugcagaagcaggaggugaagaaccugcaccagagacuggagggccaaaggccugagaacaagggcaagaacagauacaagaacauccugccuuucgaccacagcagagugauccugcagggcagagacagcaacaucccuggcagcgacuacaucaacgccaacuacaucaagaaccagcugcugggcccugacgagaacgccaagaccuacaucgccagccagggcugccuggaggccaccgugaacgacuucuggcagauggccuggcaggagaacucccgggugauugugaugaccaccagagagguggagaagggcagaaacaagugcgugccuuacuggccugaggugggcaugcagagagccuacggcccuuacagcgugaccaacugcggcgagcacgacaccaccgaguacaagcugagaacccugcaggugagcccucuggacaacggcgaucuuauucgggagaucuggcacuaccaguaccugagcuggccugaccacggcgugccuagcgagccuggcggcgugcugagcuuccuggaccagaucaaccagagacaggagagccugccucacgccggcccuaucaucgugcacugcagcgccggcaucggcagaaccggcaccaucaucgugaucgacaugcugauggagaacaucagcaccaagggccuggacugcgacaucgacauccagaagaccauccagauggugagagcccagagaagcggcauggugcagaccgaggcccaguacaaguucaucuacguggccaucgcccaguuc

SEQ ID NO: 41 - RNA, 1593 nt, Artificial Sequence
  Synthetic TCD7 nt. seq.
augggcugcggcugcagcagccacccugaggacgacuggauggagaacaucgacgugugcgagaacugccacuacccuaucgugccucuggacggcaagggcacccugcugaucagaaacggcagcgaggugcgcgacccucuggugaccuacgagggcagcaacccuccugccagcccucugcaggacaaccuggugaucgcccugcacagcuacgagccuagccacgacggcgaccugggcuucgagaagggcgagcagcugagaauccuggagcagagcggcgagugguggaaggcccagagccugaccaccggccaggagggcuucaucccuuucaacuucguggccaaggccaacagccuggagccugagccuugguucuucaagaaccugagcagaaaggacgccgagagacagcugcuggccccuggcaacacccacggcagcuuccugaucagagagagcgagagcaccgccggaagcuucagccugagcgugagagacuucgaccagaaccagggcgagguggugaagcacuacaagauuaggaaccuggacaacggcggcuucuacaucagcccuagaaucaccuucccuggccugcacgagcuggugagacacuacaccaacgccagcgacggccugugcaccagacugagcagaccuugccagacccagaagccucagaagccuuggugggaggacgagugggaggugccuagagagacgggcggaggaggcuccggcggugguggcuccggagguggcggauccuucugggaggaguucgagagccugcagaagcaggaggugaagaaccugcaccagagacuggagggccaacgcccugagaacaagggcaagaacagauacaagaacauccugccuuucgaccacagcagagugauccugcagggcagagacagcaacaucccuggcagcgacuacaucaacgccaacuauaucaagaaccaacuguugggcccugacgagaacgccaagaccuacaucgccagccagggcugccuggaggccaccgugaacgacuucuggcagauggccuggcaggagaacucucgugugaucgugaugaccaccagagagguggagaagggcagaaacaagugcgugccuuacuggccugaggugggcaugcagagagccuacggcccuuacagcgugaccaacugcggcgagcacgacaccaccgaguacaagcugagaacccugcaggugagcccucuggauaacggagaucugauuagggagaucuggcacuaccaguaccugagcuggccggaccacggcgugccuagcgagccuggcggcgugcugagcuuccuggaccagaucaaccagagacaggagagucugccucacgccggcccuaucaucgugcacugcagcgccggcaucggcagaaccggcaccaucaucgugaucgacauguugauggagaauaucagcaccaagggccuggacugcgacaucgacauccagaagaccauccagauggugagagcccagagaagcggcauggugcagaccgaggcccaguacaaguucaucuacguggccaucgcccaguuc

SEQ ID NO: 42 - RNA, 923 nt, Artificial Sequence
  Synthetic TCD8 nt. seq.
auggaggaggccauccuggugccuugcgugcugggccugcuguuauuaccuauccuggccaugcugauggcccugugcgugcacugccacagacugccuggcagcuacgacagcaccagcagcgacagccuguacccuagaggcauccaguucaagcggccucacaccguggccccuuggccuccugccuacccuccugugaccaguuauccuccucugagccagccugaccuacuaccuaucccuagaagcccucagccucugggcggcagccacagaaccccuagcagcagaagagacagcgacggcgccaacagcguggccagcuacgagaacgagggcgccagcggcaucagaggagcacaggccggcuggggcguguggggcccuagcuggaccagacugaccccugugagccugccuccagaaccugcuugcgaggacgccgacgaggacgaggacgacuaccacaacccuggcgugaccuacgcccaguugcugccugacagcaccccugccaccagcaccgccgccccuagcgccccugcccugucaaccccaggaauucgugauucggccuucagcauggagagcaucgacgacguuaccuacgcacaacugccugagagcggcgagagcgccgaggccagccuggacggcagcagagaggucaccuacgcacagcugagccaggagcugcacccuggcgccgccaagaccgagccugccgcucuuagcagccaggaggccgaggagguggaggaggagggcgccccugacuacgagaaccugcaggagcugaacaucccuaacccuuuguugggucuggacugauaauaggcuggagccucgguggccuagcuucuugccccuugggccuccccccagccccuccuccccuuccugcacccguacccccgugg

SEQ ID NO: 43 - RNA, 804 nt, Artificial Sequence
  Synthetic TCD9 nt. seq.
auggaggaggccauccuggugccuugcgugcugggccugcugcugcugccuauccuggccaugcugauggcccugugcgugcacugccacagacugccuggcagcuacgacagcaccagcagcgacagccuguacccuagaggcauccaguucaagcggccucacaccguggccccuuggccuccugccuacccuccugugaccagcuacccuccucugagccagccugaccugcucccuaucccuagaagcccucagccucugggcggcagccacagaaccccuagcagcagaagagacagcgacggcgccaacagcguggccagcuacgagaacgagggcgccagcggcaucagaggcgcucaggcaggcuggggcguguggggcccuagcuggaccagacugaccccugugagccugccuccagagccugccugcgaggacgccgacgaggacgaggacgacuaccacaacccuggcaucaccuacgccgccgugcugccugacagcaccccugccaccagcaccgccgccccuagcgccccugcccugagcacgccuggaaucagagacagcgccuucagcauggagagcaucgacgauauuacauacgcugccgugccugagagcggcgagagcgccgaggccagccuggacggcagcagagagauuacauacgcagccgugagccaggagcugcacccuggcgccgccaagaccgagccugccgcccugagcagccaggaggccgaggagguggaggaggagggcgccccugacuacgagaaccugcaggagcugaac

SEQ ID NO: 44 - RNA, 1089 nt, Artificial Sequence
  Synthetic TCD10 nt. seq.
auggaggaggccauccuggugccuugcgugcugggccugcuauuacuuccuauccuggccaugcugauggcccugugcgugcacugccacagacugccuggcagcuacgacagcaccagcagcgacagccuguacccuagaggcauccaguucaagaggccucacaccguggccccuuggccuccugccuacccuccugugaccucuuacccgccacugagccagccugaccuauugccaauaccuagaagcccucagccucugggcggcagccacagaaccccuagcagcagaagagacagcgacggcgccaacagcguggcgagcuacgagaacgagggcgccagcggcaucagaggugcccaggcaggcuggggcguguggggcccuagcuggaccagacugaccccugugagccugccuccagagccugcuugcgaggacgccgacgaggacgaagacgacuaccacaacccuggcgcccugguggugcugccugacuccacaccugccaccagcaccgccgccccuagcgccccugcccugagcacuccugguauucgugauagcgccuucagcauggagagcaucgacgacgccgugaacgugccugagagcggcgagagcgccgaggcuucccucgacggcagcagagaggcugugaacgugagccaggagcugcauccaggugcagccaagaccgaaccugccgcucugagcagucaggaggccgaggagguggaagaggagggugcaccagacuacgagaaucugcaggaguuaaaccacagacagaaccagaucaagcagggcccuccuaggucuaaggacgaggagcagaagccucagcagcggccggauuuggccguggacgugcuggagagaaccgccgacaaggccaccguuaacggauugccagagaaggacagagagacugacaccagcgcacuggccgcggguuccucucaggaggugaccuacgcccagcucgaucacugggcccugacccagaggacugccagagccgugaguccacagagcaccaagccuauggccgaaagcauuacuuacgccgccguggccagacac

SEQ ID NO: 45 - RNA, 1632 nt, Artificial Sequence
  Synthetic TCD11 nt. seq.
auggaggaggccauccuggugccuugcgugcugggccugcuccucuugccuauccuggccaugcugauggcccugugcgugcacugccacagacugccuggcagcuacgacagcaccagcagcgacagccuguacccuagaggcauccaguucaagcgcccucacaccguggccccuuggccuccugccuacccuccugugaccaguuacccgccacugagccagccugaccucuuaccuaucccuagaagcccucagccucugggcggcagccacagaaccccuagcagcagaagagacagcgacggcgccaacagcguggcuucguacgagaacgagggcgccagcggcaucagaggagcucaagcugguuggggcguguggggcccuagcuggaccagacugaccccugugagccugccuccggagccagcgugcgaggacgccgacgaggacgaggacgacuaccacaacccuggcgcccugguggugcugccugacucuacuccugccaccagcaccgccgccccuagcgccccugcccugagcacgccuggcauccgcgacucggccuucagcauggagagcaucgacgacgccgugaacgugccugagagcggcgagagcgccgaggccucuuuagacggcagcagagaggccguaaacgucagccaggagcugcacccaggugcagccaagaccgaaccggcugcccucuccagucaagaagccgaggagguagaagaggagggugcgccagacuacgagaaucugcaggaacucaacggaggagguggcagcgguggcggcggaagcuucugggaggaguucgagagccugcagaagcaggaggugaagaaccuucaccagagacuggagggccaacgcccugagaacaagggcaagaacagauacaagaacauccugccuuucgaccacagcagagugauccugcagggcagagauuccaacaucccuggcucagacuacaucaacgccaauuacaucaagaaccaguugcucggaccugacgagaacgcuaagacuuacaucgccagccagggcugccuggaggccacugugaacgacuucuggcagauggcuuggcaagagaauucucggguuauugugaugaccacacgugaaguugagaagggcagaaacaagugcgugccuuacuggccugaggugggcaugcagagagccuacggcccuuacagcgugaccaacugcggcgagcacgacaccaccgaguacaagcugagaacccugcaggugagcccucuggacaacggcgaccugaucagagagaucuggcacuaccaguaccugaguuggccagaccacggcgugccuagcgagccuggcggcgugcugagcuuccuggaccaaaucaaccagcgccaagagucucucccacacgccggcccuaucaucgugcauugcagcgccggcaucggcagaaccggcaccauuaucgugaucgauauguugauggagaacaucagcaccaagggccuggacugcgacaucgacauccagaagaccauccagauggugagagcccagagaagcggcauggugcagaccgaggcccaguacaaguucaucuacguggccaucgcccaguuc

SEQ ID NO: 46 - RNA, 981 nt, Artificial Sequence
  Synthetic TCD12 nt. seq.
auggaggaggccauccuggugccuugcgugcugggccugcugcugcugccuauccuggccaugcugauggcccugugcgugcacugccacagacugccuggcagcuacgacaguggcuucggaggcggaggcucugguggcgguggcucuggagguggaggcaguuucugggaggaguucgagagccugcagaagcaggaggugaagaaccugcaccagagacuggagggccaacggccugagaacaagggcaagaacagauacaagaacauccugccuuucgaccacagcagagugauccugcagggcagagacagcaacaucccuggcagcgacuacaucaacgccaacuacaucaagaaccagcugcugggcccugacgagaacgccaagaccuacaucgccagccagggcugccuggaggccaccgugaacgacuucuggcagauggccuggcaggagaacucaagggugauagugaugaccaccagagagguggagaagggcagaaacaagugcgugccuuacuggccugaggugggcaugcagagagccuacggcccuuacagcgugaccaacugcggcgagcacgacaccaccgaguacaagcugagaacccugcaggugagcccucuggacaacggcgaccugaucagagagaucuggcacuaccaguaccugagcuggccugaccacggcgugccuagcgagccuggcggcgugcugagcuuccuggaccagaucaaccagagacaggagagccugccucacgccggcccuaucaucgugcacugcagcgccggcaucggcagaaccggcaccaucaucgugaucgacaugcugauggagaacaucagcaccaagggccuggacugcgacaucgacauccagaagaccauccagauggugagagcccagagaagcggcauggugcagaccgaggcccaguacaaguucaucuacguggccaucgcccaguuc

SEQ ID NO: 47 - RNA, 417 nt, Artificial Sequence
  Synthetic TCD13 nt. seq.
auggaggaggccauccuggugccuugcgugcugggccugcugcugcugccuauccuggccaugcugauggcccugugcgugcacugccacagacugccuggcagcuacgacagccacagacagaaccagaucaagcagggcccuccuagaagcaaggacgaggagcagaagccucagcagagaccugaccuggccguggacgugcuggagagaaccgccgacaaggccaccgugaacggccugccugagaaggacagagagaccgacaccagcgcccuggccgccggcagcagccaggaggugaccuacgcccagcuggaccacugggcccugacccagagaaccgccagagccgugagcccucagagcaccaagccuauggccgagagcaucaccuacgccgccguggccagacac

SEQ ID NO: 48 - RNA, 1449 nt, Artificial Sequence
  Synthetic TCD14 nt. seq.
auggaggaggccauccuggugccuugcgugcugggccugcuccugcucccuauccuggccaugcugauggcccugugcgugcacugccacagacugccuaugagcgccauccaggccgccuggccuagcggcaccgagugcaucgccaaguacaacuuccacggcaccgccgagcaggaccugccuuucugcaagggcgacgugcugaccaucguggccgugaccaaggacccuaacgccuacaaggccaagaacaaggugggcagagagggcaucaucccugccaacuacgugcagaagagagagggcgugaaggccggcaccaagcugagccugaugccuugguuccacggcaagaucaccagagagcaggccgagagacugcuguacccuccugagacgggccuguuccuggugaaggagagcaccaacuacccuggcgacuacacucuuugcgugagcugcgacggcaagguggagcacuacagaaucauguaccacgccaguaagcugagcaucgacgaggagguguacuucgagaaccugaugcagcugguggcccacuacaccagcgacgccgacggccugugcaccagacugaucaagccuaaggugauggagggcaccguggccgcccaggacgaguucuacagaagcggcugggcccugaacaugaaggagcugaagcugcugcagaccaucggaaagggcgaguucggcgacgugaugcugggcgauuacagaggcaauaaggucgcuguuaagugcaucaagaacgacgccaccgcccaggccuuccuggccgaggccagcgugaugacccagcugagacacagcaaccuggugcagcugcugggcgugaucguggaggagaagggcggccuguacaucgugaccgaguacauggccaagggcagccugguggacuaccugagaagcagaggcagaucuguucugggcggcgacugccugcugaaguucagccuggacgugugcgaggccauggaguaccuggagggcaacaacuucgugcaccgggaucuggccgccagaaacgugcuggugagcgaggacaacguggccaaggugagcgacuucggccuaacgaaggaggccagcagcacccaggacaccggcaagcugccugugaaguggaccgccccugaggcccugagagagaagaaguucagcaccaagagcgacguguggagcuucggcauccugcugugggagaucuacuccuucggcagagugccuuacccuagaaucccucugaaggacguggugccuagaguugagaagggcuacaagauggacgccccugacggcugcccuccugccguguacgaggugaugaagaacugcuggcaccuggacgccgccaugcgaccuagcuuccugcagcugagagagcagcuggagcacaucaagacccacgagcugcaccug

SEQ ID NO: 49 - RNA, 886 nt, Artificial Sequence
  Synthetic TCD15 nt. seq.
augggcccugccggcagccugcugggcagcggccagaugcagaucacccuguggggcagccuggccgccguggccaucuucuucgugaucaccuuccugaucuuccugugcagcagcugcgacagagagaagaagccuagaaugagcgccauccaggccgccuggccuagcggcaccgagugcaucgccaaguacaacuuccacggcaccgccgagcaggaccugccuuucugcaagggcgacgugcugaccaucguggccgugaccaaggacccuaacgccuacaaggccaagaacaaggugggcagagagggcaucaucccugccaacuacgugcagaagagagagggcgugaaggccggcaccaagcugagccugaugccuugguuccacggaaagaucaccagagagcaggccgagagacugcuguacccuccugaaacuggccuguuccuggugaaggagagcaccaacuacccuggcgacuacacccugugcgugagcugcgacggcaagguggagcacuacagaaucauguaccacgccagcaaguuaagcaucgacgaggagguguacuucgagaaccugaugcagcugguggcccacuacaccagcgacgccgacggccugugcaccagacugaucaagccuaaggugauggagggcaccguggccgcccaggacgaguucuacagaagcggcugggcccugaacaugaaggagcugaagcugcugcagaccaucggcaagggugaguucggcgacgugaugcugggagacuacagaggcaauaagguagcaguaaagugcaucaagaacgacgccaccgcccaggccuuccuggccgaggccagcgugaugacccagcugagacacagcaaccugg

SEQ ID NO: 50 - RNA, 1500 nt, Artificial Sequence
  Synthetic TCD16 nt. seq.
augggcugcggcugcagcagccacccugaggacgacuggauggagaacaucgacgugugcgagaacugccacuacccuaucgugccucuggacggcaagggcacccugcugaucagaaacggcagcgaggugcgagauccucuggugaccaugagcgccauccaggccgccuggccuagcggcaccgagugcaucgccaaguacaacuuccacggcaccgccgagcaggaccugccuuucugcaagggcgacgugcugaccaucguggccgugaccaaggacccuaacgccuacaaggccaagaacaaggugggcagagagggcaucaucccugccaacuacgugcagaagagagagggcgugaaggccggcaccaagcugagccugaugccuugguuccacggcaagaucaccagagagcaggccgagagacugcuguacccuccugagacuggccuguuccuggugaaggagagcaccaacuacccuggcgacuacacccugugcgugagcugcgacggaaagguggagcacuacagaaucauguaccacgccucuaagcucagcaucgacgaggagguguacuucgagaaccugaugcagcugguggcccacuacaccagcgacgccgacggccugugcaccagacugaucaagccuaaggugauggagggcaccguggccgcccaggacgaguucuacagaagcggcugggcccugaacaugaaggagcugaagcugcugcagaccaucggaaagggcgaguucggcgacgugaugcugggagacuauagaggcaauaagguagccgucaagugcaucaagaacgacgccaccgcccaggccuuccuggccgaggccagcgugaugacccagcugagacacagcaaccuggugcagcugcugggcgugaucguggaggagaagggcggccuguacaucgugaccgaguacauggccaagggcagccugguggacuaccugagaagcagaggcagaagcgugcugggcggcgacugccugcugaaguucagccuggacgugugcgaggccauggaguaccuggagggcaacaacuucgugcaccgggaucuggccgccagaaacgugcuggugagcgaggacaacguggccaaggugagcgacuucggccuaacaaaggaggccagcagcacccaggacaccggcaagcugccugugaaguggaccgccccugaggcccugagagagaagaaguucagcaccaagagcgacguguggagcuucggcauccugcugugggagaucuacucauucggcagagugccuuacccuagaaucccucugaaggacguggugccuagagucgagaagggcuacaagauggacgccccugacggcugcccuccugccguguacgaggugaugaagaacugcuggcaccuggacgccgccaugcgaccuagcuuccugcagcugagagagcagcuggagcacaucaagacccacgagcugcaccug

SEQ ID NO: 51 - RNA, 1500 nt, Artificial Sequence
  Synthetic TCD17 nt. seq.
augggcugcgugcagugcaaggacaaggaggccaccaagcugaccgaggagagagacggcagccugaaccagagcagcggcuacagauacggcaccgacccuaccccucagcacuacccuagcuucggcgugaccagcaucccuaacuacaugagcgccauccaggccgccuggccuagcggcaccgagugcaucgccaaguacaacuuccacggcaccgccgagcaggaccugccuuucugcaagggcgacgugcugaccaucguggccgugaccaaggacccuaacgccuacaaggccaagaacaaggugggcagagagggcaucaucccugccaacuacgugcagaagagagagggcgugaaggccggaacaaaguuaagccugaugccuugguuccacggcaagaucaccagagagcaggccgagagacugcuguacccuccugagacuggccuguuccuggugaaggagagcaccaacuacccuggcgacuacacccugugcgugagcugcgacggcaagguggagcacuacagaaucauguaccacgccucuaagcucagcaucgacgaggagguguacuucgagaaccugaugcagcugguggcccacuacaccagcgacgccgacggccugugcaccagacugaucaagccuaaggugauggagggcaccguggccgcccaggacgaguucuacagaagcggcugggcccugaacaugaaggagcugaagcugcugcagaccaucgguaagggugaguucggcgacgugaugcugggcgacuauagaggcaauaagguggcugugaagugcaucaagaacgacgccaccgcccaggccuuccuggccgaggccagcgugaugacccagcugagacacagcaaccuggugcagcugcugggcgugaucguggaggagaagggcggccuguacaucgugaccgaguacauggccaagggcagccugguggacuaccugagaagcagaggcagaagcgugcugggcggcgacugccugcugaaguucagccuggacgugugcgaggccauggaguaccuggagggcaacaacuucgugcaccgagaucuggccgccagaaacgugcuggugagcgaggacaacguggccaaggugagcgacuucggccuuacuaaggaagcaagcagcacccaggacaccggcaagcugccaguaaaguggaccgccccugaggcccugagagagaagaaguucagcaccaagagcgacguguggagcuucggcauccugcugugggagaucuacucuuucggaagagugccuuacccuagaaucccucugaaggacguggugccuagaguagagaagggcuacaagauggacgccccugacggcugcccuccugccguguacgaggugaugaagaacugcuggcaccuggacgccgccaugcggccuagcuuccugcagcugagagagcagcuggagcacaucaagacccacgagcugcaccug

SEQ ID NO: 52 - RNA, 1380 nt, Artificial Sequence
  Synthetic TCD18 nt. seq.
augggcagcaacaagagcaagccuaaggacaugagcgccauccaggccgccuggccuagcggcaccgagugcaucgccaaguacaacuuccacggcaccgccgagcaggaccugccuuucugcaagggcgacgugcugaccaucguggccgugaccaaggacccuaacgccuacaaggccaagaacaaggugggcagagagggcaucaucccugccaacuacgugcagaagagagagggcgugaaggccggcaccaagcugagccugaugccuugguuccacggaaagaucaccagagagcaggccgagagacugcuguacccuccugagacaggccuguuccuggugaaggagagcaccaacuacccuggcgacuacacccugugcgugagcugcgacggcaagguggagcacuacagaaucauguaccacgccaguaagcucagcaucgacgaggagguguacuucgagaaccugaugcagcugguggcccacuacaccagcgacgccgacggccugugcaccagacugaucaagccgaaggugauggagggcaccguggccgcccaggacgaguucuacagaagcggcugggcccugaacaugaaggagcugaagcugcugcagaccaucggaaagggagaguucggcgacgugaugcugggugacuacagaggcaacaagguagcugucaagugcaucaagaacgacgccaccgcccaggccuuccuggccgaggccagcgugaugacccagcugagacacagcaaccuggugcagcugcugggcgugaucguggaggagaagggcggccuguacaucgugaccgaguacauggccaagggcagccugguggacuaccugagaagcagaggcagaagcgugcugggcggcgacugccugcugaaguucagccuggacgugugcgaggccauggaguaccuggagggcaacaacuucgugcaccgagaucuggccgccagaaacgugcuggugagcgaggacaacguggccaaggugagcgacuucggcuugacuaaggaggccagcagcacccaggacaccggcaagcugccugugaaguggaccgccccugaggcccugagagagaagaaguucagcaccaagagcgacguguggagcuucggcauccugcugugggagaucuacucuuucgguagagugccuuacccuagaaucccucugaaggacguggugccuagaguugagaagggcuacaagauggacgccccugacggcugcccuccugccguguacgaggugaugaagaacugcuggcaccuggacgccgccaugaggccuagcuuccugcagcugagagagcagcuggagcacaucaagacccacgagcugcaccug

SEQ ID NO: 53 - RNA, 864 nt, Artificial Sequence
  Synthetic TCD19 nt. seq.
auggaggaggccauccuggugccuugcgugcugggccugcugcugcugccuauccuggccaugcugauggcccugugcgugcacugccacagacugccucugaagcugcugcagaccaucggcaagggcgaguucggcgacgugaugcugggcgacuacagaggcaacaagguggccgugaagugcaucaagaacgacgccaccgcccaggccuuccuggccgaggccagcgugaugacccagcugagacacagcaaccuggugcagcugcugggcgugaucguggaggagaagggcggccuguacaucgugaccgaguacauggccaagggcagccugguggacuaccugagaagcagaggcagaagugugcuaggcggcgacugccugcugaaguucagccuggacgugugcgaggccauggaguaccuggagggcaacaacuucgugcaccgcgaccuggccgccagaaacgugcuggugagcgaggacaacguggccaaggugagcgacuucggccugaccaaggaggccagcagcacccaggacaccggcaagcugccugugaaguggaccgccccugaggcccugagagagaagaaguucagcaccaagagcgacguguggagcuucggcauccugcugugggagaucuacagcuucggcagagugccuuacccuagaaucccucugaaggacguggugccuagaguggagaagggcuacaagauggacgccccugacggcugcccuccugccguguacgaggugaugaagaacugcuggcaccuggacgccgccaugcgaccuagcuuccugcagcugagagagcagcuggagcacaucaagacccacgagcugcac

SEQ ID NO: 54 - RNA, 906 nt, Artificial Sequence
  Synthetic TCD20 nt. seq.
augggcccugccggcagccugcugggcagcggccagaugcagaucacccuguggggcagccuggccgccguggccaucuucuucgugaucaccuuccugaucuuccugugcagcagcugcgacagagagaagaagccuagacugaagcugcugcagaccaucggcaagggcgaguucggcgacgugaugcugggcgacuacagaggcaacaagguggccgugaagugcaucaagaacgacgccaccgcccaggccuuccuggccgaggccagcgugaugacccagcugagacacagcaaccuggugcagcugcugggcgugaucguggaggagaagggcggccuguacaucgugaccgaguacauggccaagggcagccugguggacuaccugagaagcagaggcagaagcgugcugggcggcgacugccugcugaaguucagccuggacgugugcgaggccauggaguaccuggagggcaacaacuucgugcacagagaccuggccgccagaaacgugcuggugagcgaggacaacguggccaaggugagcgacuucggccugaccaaggaggccagcagcacccaggacaccggcaagcugccugugaaguggaccgccccugaggcccugagagagaagaaguucagcaccaagagcgacguguggagcuucggcauccugcugugggagaucuacagcuucggcagagugccuuacccuagaaucccucugaaggacguggugccuagaguggagaagggcuacaagauggacgccccugacggcugcccuccugccguguacgaggugaugaagaacugcuggcaccuggacgccgccaugagaccuagcuuccugcagcugagagagcagcuggagcacaucaagacccacgagcugcac

SEQ ID NO: 55 - RNA, 915 nt, Artificial Sequence
  Synthetic TCD21 nt. seq.
augggcugcggcugcagcagccacccugaggacgacuggauggagaacaucgacgugugcgagaacugccacuacccuaucgugccucuggacggcaagggcacccugcugaucagaaacggcagcgaggugagagacccucuggugacccugaagcugcugcagaccaucggcaagggcgaguucggcgacgugaugcugggcgacuacagaggcaacaagguggccgugaagugcaucaagaacgacgccaccgcccaggccuuccuggccgaggccagcgugaugacccagcugagacacagcaaccuggugcagcugcugggcgugaucguggaggagaagggcggccuguacaucgugaccgaguacauggccaagggcagccugguggacuaccugagaagcagaggcagaagcgugcugggcggcgacugccugcugaaguucagccuggacgugugcgaggccauggaguaccuggagggcaacaacuucgugcacagagaccuggccgccagaaacgugcuggugagcgaggacaacguggccaaggugagcgacuucggccugaccaaggaggccagcagcacccaggacaccggcaagcugccugugaaguggaccgccccugaggcccugagagagaagaaguucagcaccaagagcgacguguggagcuucggcauccugcugugggagaucuacagcuucggcagagugccuuacccuagaaucccucugaaggacguggugccuagaguggagaagggcuacaagauggacgccccugacggcugcccuccugccguguacgaggugaugaagaacugcuggcaccuggacgccgccaugagaccuagcuuccugcagcugagagagcagcuggagcacaucaagacccacgagcugcac

SEQ ID NO: 56 - RNA, 915 nt, Artificial Sequence
  Synthetic TCD22 nt. seq.
augggcugcgugcagugcaaggacaaggaggccaccaagcugaccgaggagagagacggcagccugaaccagagcagcggcuacagauacggcaccgacccuaccccucagcacuacccuagcuucggcgugaccagcaucccuaacuaccugaagcugcugcagaccaucggcaagggcgaguucggcgacgugaugcugggcgacuacagaggcaacaagguggccgugaagugcaucaagaacgacgccaccgcccaggccuuccuggccgaggccagcgugaugacccagcugagacacagcaaccuggugcagcugcugggcgugaucguggaggagaagggcggccuguacaucgugaccgaguacauggccaagggcagccugguggacuaccugagaagcagaggcagaagcgugcugggcggcgacugccugcugaaguucagccuggacgugugcgaggccauggaguaccuggagggcaacaacuucgugcacagagaccuggccgccagaaacgugcuggugagcgaggacaacguggccaaggugagcgacuucggccugaccaaggaggccagcagcacccaggacaccggcaagcugccugugaaguggaccgccccugaggcccugagagagaagaaguucagcaccaagagcgacguguggagcuucggcauccugcugugggagaucuacagcuucggcagagugccuuacccuagaaucccucugaaggacguggugccuagaguggagaagggcuacaagauggacgccccugacggcugcccuccugccguguacgaggugaugaagaacugcuggcaccuggacgccgccaugagaccuagcuuccugcagcugagagagcagcuggagcacaucaagacccacgagcugcac

SEQ ID NO: 57 - RNA, 795 nt, Artificial Sequence
  Synthetic TCD23 nt. seq.
augggcagcaacaagagcaagccuaaggaccugaagcugcugcagaccaucggcaagggcgaguucggcgacgugaugcugggcgacuacagaggcaacaagguggccgugaagugcaucaagaacgacgccaccgcccaggccuuccuggccgaggccagcgugaugacccagcugagacacagcaaccuggugcagcugcugggcgugaucguggaggagaagggcggccuguacaucgugaccgaguacauggccaagggcagccugguggacuaccugagaagcagaggcagaagcgugcugggcggcgacugccugcugaaguucagccuggacgugugcgaggccauggaguaccuggagggcaacaacuucgugcacagagaccuggccgccagaaacgugcuggugagcgaggacaacguggccaaggugagcgacuucggccugaccaaggaggccagcagcacccaggacaccggcaagcugccugugaaguggaccgccccugaggcccugagagagaagaaguucagcaccaagagcgacguguggagcuucggcauccugcugugggagaucuacagcuucggcagagugccuuacccuagaaucccucugaaggacguggugccuagaguggagaagggcuacaagauggacgccccugacggcugcccuccugccguguacgaggugaugaagaacugcuggcaccuggacgccgccaugagaccuagcuuccugcagcugagagagcagcuggagcacaucaagacccacgagcugcac

SEQ ID NO: 58 - RNA, 915 nt, Artificial Sequence
  Synthetic TCD24 nt. seq.
auggaggaggccauccuggugccuugcgugcugggccugcugcugcugccuauccuggccaugcugauggcccugugcgugcacugccacagacugccuuucugggaggaguucgagagccugcagaagcaggaggugaagaaccugcaccagagacuggagggccagcgcccugagaacaagggcaagaacagauacaagaacauccugccuuucgaccacagcagagugauccugcagggcagagacagcaacaucccuggcagcgacuacaucaacgccaacuacaucaagaaccagcugcugggcccugacgagaacgccaagaccuacaucgccagccagggcugccuggaggccaccgugaacgacuucuggcagauggccuggcaggagaauucccgcguaaucgugaugaccaccagagagguggagaagggcagaaacaagugcgugccuuacuggccugaggugggcaugcagagagccuacggcccuuacagcgugaccaacugcggcgagcacgacaccaccgaguacaagcugagaacccugcaggugagcccucuggacaacggcgaccugaucagagagaucuggcacuaccaguaccugagcuggccugaccacggcgugccuagcgagccuggcggcgugcugagcuuccuggaccagaucaaccagagacaggagagccugccucacgccggcccuaucaucgugcacugcagcgccggcaucggcagaaccggcaccaucaucgugaucgacaugcugauggagaacaucagcaccaagggccuggacugcgacaucgacauccagaagaccauccagauggugagagcccagagaagcggcauggugcagaccgaggcccaguacaaguucaucuacguggccaucgcccaguuc

SEQ ID NO: 59 - RNA, 957 nt, Artificial Sequence
  Synthetic TCD25 nt. seq.
augggcccugccggcagccugcugggcagcggccagaugcagaucacccuguggggcagccuggccgccguggccaucuucuucgugaucaccuuccugaucuuccugugcagcagcugcgacagagagaagaagccuagauucugggaggaguucgagagccugcagaagcaggaggugaagaaccugcaccagagacuggagggccagcgaccugagaacaagggcaagaacagauacaagaacauccugccuuucgaccacagcagagugauccugcagggcagagacagcaacaucccuggcagcgacuacaucaacgccaauuacaucaagaaccagcugcugggcccugacgagaacgccaagaccuacaucgccagccagggcugccuggaggccaccgugaacgacuucuggcagauggccuggcaggagaacagucgcgugaucgugaugaccaccagagagguggagaagggcagaaacaagugcgugccuuacuggccugaggugggcaugcagagagccuacggcccuuacagcgugaccaacugcggcgagcacgacaccaccgaguacaagcugagaacccugcaggugagcccucuggacaacggcgaccugaucagagagaucuggcacuaccaguaccugagcuggccugaccacggcgugccuagcgagccuggcggcgugcugagcuuccuggaccagaucaaccagagacaggaaagucugccucacgccggcccuaucaucgugcacugcagcgccggcaucggcagaaccggcaccaucaucgugaucgacaugcugauggagaacaucagcaccaagggccuggacugcgacaucgacauccagaagaccauccagauggugagagcccagagaagcggcauggugcagaccgaggcccaguacaaguucaucuacguagcaaucgcccaguuc

SEQ ID NO: 60 - RNA, 966 nt, Artificial Sequence
  Synthetic TCD26 nt. seq.
augggcugcggcugcagcagccacccugaggacgacuggauggagaacaucgacgugugcgagaacugccacuacccuaucgugccucuggacggcaagggcacccugcugaucagaaacggcagcgaggugagggacccucuggugaccuucugggaggaguucgagagccugcagaagcaggaggugaagaaccugcaccagagacuggagggccaaaggccugagaacaagggcaagaacagauacaagaacauccugccuuucgaccacagcagagugauccugcagggcagagacagcaacaucccuggcagcgacuacaucaacgccaacuacaucaagaaccagcugcugggcccugacgagaacgccaagaccuacaucgccagccagggcugccuggaggccaccgugaacgacuucuggcagauggccuggcaggagaacuccagggucauugugaugaccaccagagagguggagaagggcagaaacaagugcgugccuuacuggccugaggugggcaugcagagagccuacggcccuuacagcgugaccaacugcggcgagcacgacaccaccgaguacaagcugagaacccugcaggugagcccucuggacaacggcgaccugaucagagagaucuggcacuaccaguaccugagcuggccugaccacggcgugccuagcgagccuggcggcgugcugagcuuccuggaccagaucaaccagagacaggagagccugccucacgccggcccuaucaucgugcacugcagcgccggcaucggcagaaccggcaccaucaucgugaucgacaugcugauggagaauaucagcaccaagggccuggacugcgacaucgacauccagaagaccauccagauggugagagcccagagaagcggcauggugcagaccgaggcccaguacaaguucaucuacguggccaucgcccaguuc

SEQ ID NO: 61 - RNA, 966 nt, Artificial Sequence
  Synthetic TCD27 nt. seq.
augggcugcgugcagugcaaggacaaggaggccaccaagcugaccgaggagagagacggcagccugaaccagagcagcggcuacagauacggcaccgacccuaccccucagcacuacccuagcuucggcgugaccagcaucccuaacuacuucugggaggaguucgagagccugcagaagcaggaggugaagaaccugcaccagagacuggagggccagcggccugagaacaagggcaagaacagauacaagaacauccugccuuucgaccacagcagagugauccugcagggcagagacagcaacaucccuggcagcgacuacaucaacgccaacuacaucaagaaccagcugcugggcccugacgagaacgccaagaccuacaucgccagccagggcugccuggaggccaccgugaacgacuucuggcagauggccuggcaggagaacagucgugugaucgugaugaccaccagagagguggagaagggcagaaacaagugcgugccuuacuggccugaggugggcaugcagagagccuacggcccuuacagcgugaccaacugcggcgagcacgacaccaccgaguacaagcugagaacccugcaggugagcccucuggacaacggcgaccugaucagagagaucuggcacuaccaguaccugagcuggccugaccacggcgugccuagcgagccuggcggcgugcugagcuuccuggaccagaucaaccagagacaggagagccugccucacgccggcccuaucaucgugcacugcagcgccggcaucggcagaaccggcaccaucaucgugaucgacaugcugauggagaacaucagcaccaagggccuggacugcgacaucgacauccagaagaccauccagauggugagagcccagagaagcggcauggugcagaccgaggcccaguacaaguucaucuacguggccaucgcccaguuc

SEQ ID NO: 62 - RNA, 846 nt, Artificial Sequence
  Synthetic TCD28 nt. seq.
augggcagcaacaagagcaagccuaaggacuucugggaggaguucgagagccugcagaagcaggaggugaagaaccugcaccagagacuggagggccaacgcccugagaacaagggcaagaacagauacaagaacauccugccuuucgaccacagcagagugauccugcagggcagagacagcaacaucccuggcagcgacuacaucaacgccaacuacaucaagaaccagcugcugggcccugacgagaacgccaagaccuacaucgccagccagggcugccuggaggccaccgugaacgacuucuggcagauggccuggcaggagaauagcaggguuauugugaugaccaccagagagguggagaagggcagaaacaagugcgugccuuacuggccugaggugggcaugcagagagccuacggcccuuacagcgugaccaacugcggcgagcacgacaccaccgaguacaagcugagaacccugcaggugagcccucuggacaacggcgaccugaucagagagaucuggcacuaccaguaccugagcuggccugaccacggcgugccuagcgagccuggcggcgugcugagcuuccuggaccagaucaaccagagacaggagagccugccucacgccggcccuaucaucgugcacugcagcgccggcaucggcagaaccggcaccaucaucgugaucgacaugcugauggagaacaucagcaccaagggccuggacugcgacaucgacauccagaagaccauccagauggugagagcccagagaagcggcauggugcagaccgaggcccaguacaaguucaucuacguggccaucgcccaguuc

SEQ ID NO: 63 - RNA, 1641 nt, Artificial Sequence
  Synthetic TCD29 nt. seq.
auggaggaggccauccuggugccuugcgugcugggccugcugcuccuuccuauccuggccaugcugauggcccugugcgugcacugccacagacugccugugcggugguuccacagagaucugagcggccuggacgccgagacacugcugaagggcagaggcgugcacggcagcuuccuugcaagaccuagcagaaagaaccagggcgacuucagccugagcgugagagugggcgaccaggugacccacaucagaauccagaacagcggagauuucuacgaccuguacggcggcgagaaguucgccacccugaccgagcugguggaguacuacacccagcagcagggcgugcugcaggacagagacggcaccaucauccaccugaaguacccucugaacugcagcgacccuaccagcgagcgcugguaccacggccacaugagcggcggccaggcagagacacuccuccaggccaagggcgagccuuggaccuuccuggugagagagagucuaucccagccuggugacuucguguugagcguacucucggaccagccuaaggccggcccuggcagcccucugagagucacacacauuaaggugaugugcgagggcggcagauacaccgugggaggccuugagacuuucgacucacugacagaccucguggagcacuucaagaagaccggcaucgaagaggcaagcggcgccuucguguaccugagacagccuuacuacgccaccagagugaacgccgccgacaucgagaacagagugcuggagcugaacaagaagcaggagagcgaggacaccgccaaggcgggauucugggaggaguucgaaucucugcagaagcaagaagugaagaaccugcaccagagacuggagggccagcguccagagaacaagggcaagaacagauacaagaacauccugccuuucgaccacagcagagugauccugcagggcagggacagcaacaucccuggaagugacuacaucaacgccaauuacauuaagaaucagcugcugggaccugacgagaacgcuaagacguacaucgccagccagggcugccuggaggccaccgugaacgacuucuggcagauggccuggcaggagaauucacgugugauugugaugacaacccgagagguggagaagggccguaacaagugcgugccuuacuggccugaggugggcaugcagagagccuacggcccuuacagcgugaccaacugcggcgagcacgacaccaccgaguacaagcugagaacccugcaggugucaccucuugacaacggcgaccugaucagagagaucuggcacuaccaauauuuaucuuggccagaccacggcgugccuagcgagccaggcgguguccuuagcuuccuggaccagaucaaucagcgccaagagucccucccucacgcuggcccaaucaucguucacugcucugcaggcaucggcagaacugguacaauuaucgucaucgauauguuaauggagaacaucagcaccaagggucuggacugugacauugacauccagaagaccauccagaugguuagggcccagagaagcggcauggugcagaccgaggcccaguacaaguucaucuacguggccaucgcccaguuc

SEQ ID NO: 64 - RNA, 1683 nt, Artificial Sequence
  Synthetic TCD30 nt. seq.
augggcccugccggcagccugcugggcagcggccagaugcagaucacccugugggguucguuggccgccguggccaucuucuucgugaucaccuuccugaucuuccugugcagcagcugcgacagagagaagaagccuagagugcgcugguuccaccgcgaccugagcggccuggacgccgagacacugcugaagggcagaggcgugcacggcagcuuccuggccagaccuagcagaaagaaccagggcgacuucagccugagcgugagagugggcgaccaggugacccacaucagaauccagaacagcggcgauuucuacgaccuguacggcggcgagaaguucgccacccugaccgagcugguggaguacuacacccagcagcagggcgugcugcaggacagagacggcaccaucauccaccugaaguacccucugaacugcagcgacccuaccagcgagcggugguaccacggccacaugagcggcggccaggccgagacucuccuucaggccaagggcgagccuuggacauuccucgugagagagucgcugucacagccuggugauuucgugcugaguguacugagcgaccagccuaaggccggcccuggcagcccucugagagucacucacaucaaggugaugugcgagggcggcagauacaccgugggaggcuuagagacuuucgacuccuuaaccgaccuaguggaacacuucaagaagaccggcaucgaggaggccagcggcgccuucguguaccugagacagccuuacuacgccaccagagugaacgccgccgacaucgagaacagagugcuggagcugaacaagaagcaggagagcgaggacaccgccaaggcaggcuucugggaggaguucgagagucugcagaagcaggaagugaagaaccugcaccagagacuggagggccaacguccggagaacaagggcaagaacagauacaagaacauccugccuuucgaccacagcagagugauccugcaaggaagggacagcaacaucccaggaucagacuacaucaacgccaauuauaucaagaaccaacuguuaggaccugacgagaacgccaagaccuacaucgccagccagggcugccuggaggccaccgugaacgacuucuggcagauggccuggcaggagaacucgcguguuauugugaugacuacucgagagguggagaagggacggaacaagugcgugccuuacuggccugaggugggcaugcagcgggcuuacggcccuuacagcgugaccaacugcggcgagcacgacaccaccgaguacaagcugagaacccugcaggugaguccucuggacaacggcgaccugaucagagagaucuggcacuaccaguaccucaguuggccugaccacggcgugccuagcgaaccaggcggcguacuguccuuccuggaccagaucaaucagcgccaagaaucucuuccucacgccggaccgaucaucgugcacugcagugccgguauuggcagaacaggcaccauuauugucaucgacaugcugauggagaacaucagcaccaagggacuugauugugauauagacauccagaagaccauccagaugguuagagcccagagaagcggcauggugcagaccgaggcccaguacaaguucaucuacguagccauagcccaguuc

SEQ ID NO: 65 - RNA, 1692 nt, Artificial Sequence
  Synthetic TCD31 nt. seq.
augggcugcggcugcagcagccacccugaggacgacuggauggagaacaucgacgugugcgagaacugccacuacccuaucgugccucuggacggcaagggcacccugcugaucagaaacggcagcgaggugcgugacccucuggugaccgugcgcugguuccacagggaccugagcggccuggacgccgaaacguugcucaagggcagaggcgugcacggcagcuuccuggccagaccuagcagaaagaaccagggcgacuucagccugagcgugagagugggcgaccaggugacccacaucagaauccagaacagcggcgacuucuacgaccuguacggcggcgagaaguucgcuacucucaccgagcugguggaguacuacacccagcagcagggcgugcugcaggacagagacggcaccaucauccaccugaaguacccucugaacugcagcgacccuaccagcgagcgaugguaccacggccacaugagcggcggccaggccgagacacuacugcaggccaagggcgagccuuggaccuuccuggugagagagagccugagccagccuggcgacuucgugcugagugugcuuagcgaccagccuaaggccggcccuggcagcccucugagagucacacauaucaaggugaugugcgagggcggcagauacaccgugggagguuuggagacuuucgacagccugaccgaccugguggagcacuucaagaagaccggcaucgaggaggccagcggcgccuucguguaccugagacagccuuacuacgccaccagagugaacgccgccgacaucgagaacagagugcuggagcugaacaagaagcaggagagcgaggacaccgccaaggccggcuucugggaggaguucgagagccugcagaagcaagaggugaagaaccugcaccagagacuggagggacagcgaccggagaacaagggcaagaacagauacaagaacauccugccuuucgaccacagcagagugauccugcagggcagagacagcaacaucccaggaagcgacuacaucaacgccaauuauaucaagaaccagcugcugggcccugacgagaacgccaagaccuacaucgccagccagggcugccuggaggccaccgugaacgacuucuggcagauggccuggcaggagaacucucggguuaucgugaugaccaccagagagguggagaaggguagaaacaagugcgugccuuacuggccugaggugggcaugcagagagccuacggcccuuacagcgugaccaacugcggcgagcacgacaccaccgaguacaagcugagaacccugcaggugagcccucuggacaacggcgaccugaucagagagaucuggcacuaccaguaccugagcuggccugaccacggcgugccuagcgagccuggcggaguguugucguuccuggaccagaucaaccagagacaggaaaguuuaccucacgccggcccuaucaucgugcacugcagcgccggcaucggcagaacuggcacuauaaucgugaucgacauguuaauggagaauaucagcaccaagggccuggacugcgacaucgacauccagaagaccauccagauggugagagcccagagaagcggcauggugcagaccgaggcccaguacaaguucaucuacguggccaucgcccaguuc

SEQ ID NO: 66 - RNA, 1692 nt, Artificial Sequence
  Synthetic TCD32 nt. seq.
augggcugcgugcagugcaaggacaaggaggccaccaagcugaccgaggagagagacggcagccugaaccagagcagcggcuacagauacggcaccgacccuaccccucagcacuacccuagcuucggcgugaccagcaucccuaacuacgugcggugguuccacagggaucugagcggccuggacgccgaaacccugcugaagggcagaggcgugcacggcagcuuccuggccagaccuagcagaaagaaccagggcgacuucagccugagcgugagagugggcgaccaggugacccacaucagaauccagaacagcggagacuucuacgaccuguacggcggcgagaaguucgccacccucacagaacugguggaguacuacacccagcagcagggcgugcugcaggacagggacggaaccaucauccaccugaaguacccucugaacugcagcgauccaacaagcgagcggugguaccacggccacaugagcggcggccaggcugagacauuacuccaggccaagggcgagccuuggaccuuccuggugagagaguccuugagccagccuggugauuucgugcugagugugcucucugaccagccuaaggccggcccuggcagcccucugagaguuacucauaucaaggugaugugcgagggcggcagauacaccguggguggccucgagacauucgacagccugaccgaccugguggaacacuucaagaagaccggcaucgaggaagcaagcggcgccuucguguaccugagacagccuuacuacgccaccagagugaacgccgccgacaucgagaacagagugcuggagcugaacaagaagcaggagagcgaggacaccgccaaggcagguuucugggaggaguucgaaagccugcagaagcaagaagugaagaaccugcaccagagacuggagggccaacggccagagaacaagggcaagaacagauacaagaacauccugccuuucgaccacagcagagugauccuucagggccgagacagcaacaucccuggcucagacuacaucaacgccaauuacauuaagaaucagcugcugggcccugacgagaacgccaagaccuacaucgccagccagggcugccucgaagccacggugaacgacuucuggcagauggccuggcaggagaauagccgggugaucgugaugacaaccagagagguggagaaggguagaaacaagugcgugccuuacuggccugaggugggcaugcagcgggccuacggcccuuacagugugacgaacugcggcgagcacgacaccaccgaguacaagcugagaacccugcaggugucaccacuggacaacggcgaccugaucagagagaucuggcacuaccaguaucuuaguuggccggaccacggcgugccuagcgagccuggcggcguccugagcuucuuggaccagaucaaucagagacaggagucccugccucacgcgggcccgaucaucgugcauuguucugcaggcaucggcagaaccggcacuaucaucgucaucgacaugcugauggagaacaucagcaccaagggucuggacugcgacauagacauccagaagaccauccagauggugcgggcccagagaagcggcauggugcagaccgaggcccaguacaaguucaucuacguggccaucgcccaguuc

SEQ ID NO: 67 - RNA, 1572 nt, Artificial Sequence
  Synthetic TCD33 nt. seq.
augggcagcaacaagagcaagccuaaggacgugcgcugguuccaccgcgaccugagcggccuggacgccgagacacugcugaagggcagaggcgugcacggcagcuuccuggccagaccuagcagaaagaaccagggcgacuucagccugagcgugagagugggcgaccaggugacccacaucagaauccagaacagcggagauuucuacgaccuguacggcggcgagaaguucgccacccugaccgagcugguggaguacuacacccagcagcagggcgugcugcaggacagagacggcaccaucauccaccugaaguacccucugaacugcagcgacccuaccagcgagcggugguaccacggccacaugagcggcggccaggcugaaacacuccuccaagccaagggcgagccuuggaccuuccuggugagagagagccugucucagccuggugacuucgugcugucaguucuguccgaccagccaaaggccggcccuggcagcccucugagagucacccauauaaaggugaugugcgagggcggcagauacaccgugggaggcuuggaaacauucgacagucuaacagaccuugucgaacacuucaagaagaccggcaucgaggaggccagcggcgccuucguguaccugagacagccuuacuacgccaccagagugaacgccgccgacaucgagaacagagugcuggagcugaacaagaagcaggagagcgaggacaccgccaaggcgggauucugggaggaguucgaauccuuacagaagcaggaagugaagaaccugcaccagagacuggagggccagaggccagagaacaagggcaagaacagauacaagaacauccugccuuucgaccacagcagagugauccugcaaggcagggacagcaacauuccaggcucagacuacaucaacgccaacuauaucaagaaucagcugcugggcccugacgagaacgccaagaccuacaucgccagccagggcugccuggaggccaccgugaacgacuucuggcagauggccuggcaggagaacucaagagucaucgugaugacaacucgggagguggagaagggacgaaacaagugcgugccuuacuggccugaggugggcaugcagcgagcuuacggcccuuacagcgugaccaacugcggcgagcacgacaccaccgaguacaagcugagaacccugcaggugucgccacuggacaacggcgaccugaucagagagaucuggcacuaccaauauuuaucuuggccggaucacggcgugccuagcgagccuggcgguguguugaguuuccuggaccagaucaaucagcggcaggagucauuaccucacgccggaccaaucaucgugcacugcucagccggaauuggcagaacaggaaccauuaucgugaucgacaugcugauggagaacaucagcaccaagggacuggacugugauauagacauccagaagaccauccagauggugcgcgcccagagaagcggcauggugcagaccgaggcccaguacaaguucaucuacguggccaucgcccaguuc

SEQ ID NO: 68 - RNA, 237 nt, Artificial Sequence
  Synthetic TCD34 nt. seq.
auggaggccgacgcccugagcccugugggccugggccugcugcugcugccuuuccuggugacccugcuggccgcccugugcgugagaugcagagagcugccugugagcuacgacgccgugagccugagcaagaugcugaagaagagaagcccucugaccaccggcguguacgugaagaugccuccuaccgagccugagugcgagaagcaguuccagccuuacuucaucccuaucaac

SEQ ID NO: 69 - RNA, 261 nt, Artificial Sequence
  Synthetic TCD35 nt. seq.
auggaggaggccauccuggugccuugcgugcugggccugcugcugcugccuauccuggccaugcugauggcccugugcgugcacugccacagacugccuggcagcuacgacagcggcuucggcggcggcggcagcaccgccgugagccugagcaagaugcugaagaagagaagcccucugaccaccggcguguacgugaagaugccuccuaccgagccugagugcgagaagcaguuccagccuuacuucaucccuaucaac

SEQ ID NO: 70 - RNA, 975 nt, Artificial Sequence
  Synthetic TCD36 nt. seq.
auggaggaggccauccuggugccuugcgugcugggccugcugcugcugccuauccuggccaugcugauggcccugugcgugcacugccacagacugccuggcagcuacgacagcggcuucggcggcggcggcagcauggagaaggaguucgagcagaucgacaagagcggcagcugggccgccaucuaccaggacaucagacacgaggccagcgacuucccuugcagaguggccaagcugccuaagaacaagaacagaaacagauacagagacgugagcccuuucgaccacagcagaaucaagcugcaccaggaggacaacgacuacaucaacgccagccugaucaagauggaggaggcccagagaagcuacauccugacccagggcccucugccuaacaccugcggccacuucugggagauggugugggagcagaagagcagaggcguggugaugcugaacagagugauggagaagggcagccugaagugcgcccaguacuggccucagaaggaggagaaggagaugaucuucgaggacaccaaccugaagcugacccugaucagcgaggacaucaagagcuacuacaccgugagacagcuggagcuggagaaccugaccacccaggagaccagagagauccugcacuuccacuacaccaccuggccugacuucggcgugccugagagcccugccagcuuccugaacuuccuguucaaggugagagagagcggcagccugagcccugagcacggcccugugguggugcacugcagcgccggcaucggcagaagcggcaccuucugccuggccgacaccugccugcugcugauggacaagagaaaggacccuagcagcguggacaucaagaaggugcugcuggagaugagaaaguucagaaugggccugauccagaccgccgaccagcugagauucagcuaccuggccgugaucgagggcggcaagccuagcacc

SEQ ID NO: 71 - RNA, 2676 nt, Artificial Sequence
  Synthetic TCD37 nt. seq.
augagcgccgagggcuaccaguacagagcccuguacgacuacaagaaggagagagaggaggacaucgaccugcaccugggcgacauccugaccgugaacaagggcagccugguggcccugggcuucagcgacggccaggaggccagaccugaggagaucggcuggcugaacggcuacaacgagacaaccggcgagagaggcgacuucccuggcaccuacguggaguacaucggcagaaagaagaucagcccuccuaccccuaagccuagaccuccgcggccacugccuguggccccuggcagcagcaagaccgaggccgacguggagcagcagccugccccugcccugccuccaaagccuccuaagccgaccaccguggccaacaacggcaugaacaacaacaugagccugcaggacgccgagugguacuggggugacauuagccgcgaggaagugaacgagaagcugagagacaccgccgacggcaccuuccuggugagagacgccagcaccaagaugcacggcgacuacacccugacccugagaaagggcggcaacaacaagcugaucaagaucuuccacagagacggcaaguacggcuucucugauccucugaccuucagcagcgugguggaguugauaaaccacuacagaaacgagagccuggcccaguacaacccuaagcuggacgugaagcugcuguacccugugagcaaguaccagcaggaccagguggugaaggaagacaacaucgaggccgugggcaagaagcugcacgaguacaacacccaguuccaggagaagagucgcgaauacgacagacuguacgaggaguacaccagaaccagccaggagauccagaugaagagaaccgcuauagaggcguucaacgagacuaucaagauuuucgaggagcagugccagacccaggagagauacagcaaggaauacauagagaaguucaagagagaaggcaacgagaaggaaauccagagaaucaugcacaacuacgacaagcuuaagagcagaaucagcgagaucaucgacagcagaagaagacuggaggaagaccugaagaagcaggccgccgaauaucgggagaucgacaagagaaugaacagcaucaagccugaccugauccagcugcgaaagacccgagaucaguaccugauguggcugacccagaagggcgugagacagaagaagcuuaacgagugguuggguaacgagaacaccgaggaucaguauagcuuaguggaggacgacgaggaucucccucaccacgacgagaagacuuggaacgugggcaguucuaacagaaacaaggccgagaaccugcugagaggcaagcgggacgguacauuccucguacgggaaagcaguaagcagggcugcuacgccuguuccguugucguggacggcgaggugaagcacugcgugaucaacaagaccgccaccggcuacgguuucgccgagccuuacaaccuguacagcagucucaaggagcuggugcugcacuaccagcacaccagccucguccagcacaacgacuccuugaacgugacccuggccuauccaguguacgcccaacagaggagaggcggcgguggcucugguggaggagguaguacagcgaucaucaaggagauugugucacggaacgagagaagauaccaggaggacggcuucgaccuggaccugaccuacaucuacccuaacaucaucgccaugggcuucccugccgagcgucucgagggcguguauagaaacaauauugacgacguggugagauuccuggacucuaagcacaagaaccacuauaagaucuauaaucucugcgccgagaggcauuacgauaccgcuaaguucaacugcagaguagcucaguauccauucgaggaccacaacccuccucagcuggaacucauuaagccauucugcgaagauuuggaucaguggcugagcgaggacgauaaccacguggccgccauccacugcaaggccggcaagggaagaaccggcgugaugaucugcgccuaccugcugcaccgaggcaaguuccugaaggcgcaggaagcucuggacuucuacggagaagucagaacucgggauaagaagggaguaaccaucccuagccagcguagguacguguacuacuacucauaccuguugaagaaccaucuggacuacagaccuguggcacugcuguuccacaagaugauguucgaaacgauuccgauguucaguggcggcaccugcaacccucaguucguggugugccagcugaaggugaagauuuacucaagcaacagcggcccuaccagaagagaggacaaguucauguacuucgaguucccucagccucucccaguuugcggagacauaaaggucgaguucuuccauaagcagaacaagauguuaaagaaggauaagauguuccacuucugggugaacaccuucuucaucccugguccggaggagacaucagaggagguugagaacggaagucucugcgaccaggaaauugauucaaucugcagcaucgagagagccgacaacgauaaggaguaucuagugcuuacacuuacaaagaacgaucuggauaaggccaauaaggacaaggcaaauagauacuucagcccuaacuucaagguuaagcuuuacuucaccaagaca

SEQ ID NO: 72 - RNA, 4026 nt, Artificial Sequence
  Synthetic TCD38 nt. seq.
augagcgccgagggcuaccaguaccgggcccuguacgacuacaagaaggagcgggaggaggacaucgaccugcaccugggcgacauccugaccgugaacaagggcagccugguggcccugggcuucuccgacggccaggaagccaggcccgaggagaucggcuggcugaacggcuacaacgagacuaccggcgagcggggcgacuuccccggcaccuacguggaguacaucggccggaagaagaucagcccucccacucccaagccccggccuccaagaccccugcccguggcaccuggcagcagcaagaccgaggccgacguggaacagcagcccgcacccgccuugccuccuaagccgcccaagcccaccaccguggccaacaacggcaugaacaacaacaugagccugcaggacgccgagugguacuggggcgacaucagccgggaggaggugaacgagaagcugcgggacaccgccgacggcaccuuccuggugcgcgacgccagcaccaagaugcacggcgacuacacccugacccugcggaagggcggcaacaacaagcugauaaagaucuuccaccgggacggcaaguacggcuucagcgauccccugaccuucagcagcgugguggagcugaucaaccacuaccggaacgagagccuggcccaguacaaccccaagcuggacgugaagcugcuguaccccgugagcaaguaccagcaggaccagguggugaaggaggacaacaucgaggccgugggcaagaagcugcacgaguacaacacccaguuccaggagaagucucgggaguacgaccggcuguacgaggaguacacccggaccagccaggagauccagaugaagcggaccgccaucgaggccuucaacgaaaccaucaagaucuucgaggagcagugccagacccaggagcgguacagcaaggaguacaucgagaaguucaagcgggaaggcaacgagaaggagauccagcggaucaugcacaacuacgacaagcugaagucucggaucagcgagaucaucgacagccggagacggcuggaggaggaucugaagaagcaggccgccgaguaccgggagaucgacaagcggaugaacagcaucaagcccgaccugauccagcugcggaagacccgggaccaguaccugauguggcugacccagaagggcgugcggcagaagaagcugaacgaguggcugggcaacgagaacaccgaggaccaguacagccugguggaggacgacgaggaccugccccaccacgacgagaagaccuggaacgugggcagcagcaaccggaacaaggccgagaaccugcugcggggcaagcgggacggcacuuuccuggugcgggagagcagcaagcagggcugcuacgccugcagcguugugguggacggagaggugaagcacugcgugaucaacaagaccgccaccggcuacggcuucgccgagcccuacaaccuguacagcagccugaaggagcuggugcugcacuaccagcacaccagccuggugcagcacaacgacagccugaacgugacccuggccuaccccguguacgcccagcaacggaggggugguggaggaucuggcggcggcggcagugaucccgaggaggacaccguggagagcguggugagcccucccgagcugccaccccggaacauaccccugaccgccagcagcugcgaggcuaaggaggugcccuucagcaacgagaauccccgggccaccgagacgagccggcccagccugagcgaaacccuguuccagcggcugcagagcauggacaccagcggccugcccgaggagcaccugaaggccauccaggacuaccugagcacccagcuggcccaggacagcgaguucgugaagacaggcagcagcagccugccccaccugaagaagcugaccacccugcugugcaaggagcuguacggcgaggugauccggacccugcccagccuggagagccugcagcggcuguucgaccagcagcugucuccuggacugcggccucgcccacaggugcccggcgaggccaaccccaucaacauggugagcaagcugagccagcugaccagccugcugagcagcaucgaggacaaggugaaggcccugcugcacgagggccccgagagcccacaccggcccucccugauaccacccgugaccuucgaggugaaggccgagagccugggcaucccucagaagaugcagcugaagguggacguggagagcggcaagcugaucaucaagaagucaaaggacggcagcgaggacaaguucuacagccacaagaagauccugcagcugaucaagagccagaaguuccugaacaagcuggugauccugguggaaaccgagaaggagaagauacugcggaaggaguacguguucgccgacagcaagaagcgggagggcuucugccagcugcugcagcagaugaagaacaagcacagcgagcagcccgagcccgacaugaucaccaucuucaucggcaccuggaacaugggcaacgccccuccacccaagaagaucaccagcugguuccugagcaagggccagggcaagaccagggacgacagcgccgacuacaucccucacgacaucuacgugaucggcacccaggaggacccgcugagcgagaaggaguggcuggagauccugaagcacagccugcaggagaucaccagcgugaccuucaagaccguggccauccacacccuguggaacauccggaucguggugcuggccaagcccgagcacgagaaccggaucagccacaucugcaccgacaacgugaagaccggcaucgccaacacccugggcaacaagggcgccgugggcgugagcuucauguucaacggcaccagccugggcuucgugaacagccaccugaccagcggcagcgagaagaagcugcggcggaaccagaacuacaugaacauccugcgguuccuggcccugggcgacaagaagcugagccccuucaacaucacccaccgguucacccaccuguucugguucggcgaccugaacuaccggguggaccugcccaccugggaggccgaaaccaucauccagaagaucaagcagcagcaguacgccgaccugcugagccacgaccagcugcugaccgagcggcgggagcagaagguguuccugcacuucgaggaggaggagaucaccuucgccccaaccuaccgguucgagcggcugacccgggacaaguacgccuacaccaagcagaaggccaccggcaugaaguacaaccugcccagcuggugcgaccgggugcuguggaagucuuacccgcuggugcacguggugugccagagcuacggcagcaccagcgacaucaugaccagcgaccacagccccguguucgccaccuucgaggccggcgugaccagccaguucgugagcaagaacggccccggcaccguggacagccagggccagaucgaguuccugcggugcuacgccacccugaagaccaagagccagaccaaguucuaccuggaguuccacagcagcugccuggagagcuucguuaagagccaggagggcgagaacgaggagggcagcgagggcgagcugguggugaaguucggcgagacucugcccaagcugaagcccaucaucagcgaccccgaguaccugcuggaccagcacauccugaucagcaucaagagcagcgacagcgacgagagcuacggcgagggcugcaucgcccugcggcuggaggccaccgaaacccagcugcccaucuacacaccccugacccaccacggcgagcugaccggccacuuccagggcgagaucaagcugcagaccagccagggaaagacccgggagaagcuguacgacuucgugaagaccgagcgggacgagagcagcggccccaagacccucaagagccugaccagccacgaccccaugaagcagugggaggugaccagccgugcaccuccuugcagcggcagcagcaucaccgag

SEQ ID NO: 73 - RNA, 3339 nt, Artificial Sequence
  Synthetic TCD39 nt. seq.
auggacccggaagaggacaccguggagagcguggugagcccuccugaacucccuccuagaaauauaccucugacggccuccagcugcgaggcaaaggaggugcccuucucuaacgagaauccgagagccaccgagacuagcagaccuagccugucugaaacccucuuccagaggcugcagagcauggacaccagcggccugccugaggagcaucuuaaggcaauccaggacuaccugucaacacagcuggcacaggacagcgaguucgucaagacgggcucaagcucucugccucaccugaagaagcugacaaccuuacugugcaaggaacucuacggagaagugauccgcacauugcccagucuggagagucugcagagacuguuugaucaacagcugagcccgggccugaggccucggccucaggucccuggcgaggccaacccuaucaacauggugucgaaguuaucccaauuaaccagccuauuauccagcauagaggacaaggugaaggcccugcugcacgagggcccagaguccccucaccgcccaagccuuaucccuccugugacauucgagguuaaagccgagucccucgguaucccucagaagaugcagcugaaggucgacguugagucaggcaagcugauuaucaagaagucuaaggacggcagcgaagacaaguucuacagccacaagaagauccuacagcucaucaagagccagaaguuucucaauaaguuagugauccuggucgagacggagaaagagaagaucuuaagaaaggaguacguguucgccgacagcaagaagcgggagggcuucugccaguugcuucagcaaaugaagaacaagcacagcgagcagccugaaccugacaugaucacaaucuucauuggcacuuggaauauggggaacgccccuccuccuaagaagauuaccagcugguuucugagcaagggccaagggaagaccagggacgauagugcggacuacaucccucacgauauuuacgugaucggcacccaggaagacccucugagcgagaaggaguggcuggaaauacugaagcauagccugcaggagaucaccucggugaccuucaagaccguggcaauacauacccucuggaacauccggauaguugugcuagcuaagccggaacacgagaacagaaucucucauaucugcaccgacaacgugaagaccgggauugcuaacacacugggcaacaagggugcagugggagugagcuucauguucaacggcaccucacugggcuucgugaacagucaccugacaagcggcuccgagaagaaguuaagacguaaccagaauuauaugaacauccugagauuucuggcucugggagacaagaagcugucucccuucaacauaacccauagauucacccaccucuucugguuuggugaccugaauuaccgcguggaucuaccuaccugggaggcugagacuauuauacagaagauuaagcagcagcaguacgccgaccugcugagccacgaccagcugcugacagagcggagagaacagaagguguuucuccauuucgaggaggaagagaucacauucgcgccuaccuacagguucgagagauugaccagagacaaguacgccuacaccaaacagaaggccaccggcaugaaguauaauuugccaagcuggugcgacaggguguuguggaaaucauacccauugguucacgugguuugccagucuuacggcagcacgagcgacaucaugaccagcgaccacagcccuguguucgccaccuucgaggccggcgugaccagucaguucguuucuaagaacggccccggcacuguggacagccaggggcagauugaguuccucaggugcuacgcaaccuuaaagaccaagagccagaccaaauucuaccuggaguuccauagcagcugccuagaaucguucgugaagucccaagagggcgagaacgaggagggcagcgaaggcgagcuggucgugaaauuuggcgagacacugccuaagcuaaagccuaucaucagcgacccugaguaucuccuggaccagcacauacugauuucaaucaagagcagcgauucugacgaaaguuacggcgagggcugcaucgcucucagacuugaagcuacagaaacucaguuacccaucuacaccccucugacccaccacggcgagcugaccggccacuuccagggcgaaaucaaacugcagaccucccagggcaagacccgggagaagcuuuacgacuucguuaagacagagagagacgagucaucaggcccuaagacccucaagucgcuuacuucccacgauccuaugaagcagugggaagucacaucccgcgcuccucccugcagcggaagcagcaucacagaaaucggcggagguggaagcggcgguggcggcucuugguuccacggcaaacugggagccggcagggacgguagacacauagccgaaagacugcugacugaauacuguaucgagacaggcgccccugacggcucauuccugguaagagagagugagacauuugugggcgacuauacucugagcuucuggcgcaacggcaaagugcagcacugcaggauucacucccgccaggacgccgggacgccuaaguucuuccugacggauaaccugguguuugacucgcuguacgaucugaucacccacuaccagcaggucccgcugcgguguaacgaguuugaaaugagacugagugagccagugccacagaccaacgcgcacgaguccaaggagugguaucacgccagucugacgagagcccaggcagagcacaugcugaugcgugugcccagagacggugccuuucugguccgaaagagaaacgagccaaacagcuacgccaucaguuuccgcgccgagggcaagaucaagcauugccgcgugcaacaggagggacagacugucaugcuggggaauuccgaauucgacucccugguggaccucaucagcuacuacgagaagcacccucuguaccggaagaugaaacuccgguauccuauaaacgaggaagcccucgagaagauugguaccgcggaaccagauuacggagcucuguacgagggcaggaauccgggcuucuacguggaagccaauccaaugcccacauucaagugugccgugaaggcucuauucgacuacaaggcccagcgcgaggacgaguugacguucauuaagagcgcaaucauccagaacguggagaagcaggagggcgguugguggaggggugacuacggcgguaagaagcagcugugguucccuaguaauuacgucgaggaaauggucaac

SEQ ID NO: 74 - RNA, 3339 nt, Artificial Sequence
  Synthetic TCD40 nt. seq.
augugguuccacggaaagcuaggagccgguagggacggaagacauauagccgagagguugcugacagaguacuguauugaaaccggagccccugacggcucguucuuagucagagaaucugagacauucgugggugacuauacccugucguucuggcgaaacggcaaggugcagcacugucgcauccacuccagacaggacgcuggaacaccuaaguucuuccuuaccgacaaucuggucuucgacucucuuuacgauuugauaacccauuaccagcaggucccucugcgcugcaacgaguucgaaaugagacucagugaaccugugccucagacuaacgcucacgagaguaaggagugguaucacgcuucccucacccgcgcacaggcugaacacaugcucaugagggucccacgcgacggagcauuccuggugaggaagcguaacgaaccaaauuccuacgccauuagcuuccgggcagagggcaagauaaagcacugccgaguucagcaggagggccagacagugaugcuaggaaauucagaguucgacucacuuguugaucucauuagcuacuacgagaagcacccuuuguacagaaagaugaagcugcgguauccaaucaacgaggaggcccuggagaagauuggcacugcugaaccugacuacggagcccuguacgagggccgcaauccgggauucuacgucgaggcgaauccgaugccaacauucaagugcgcaguuaaggcccuuuucgauuacaaggcccagcgggaggacgagcucacuuucauuaagucugcgaucauccagaacgucgagaagcaagagggaggcugguggcgcggagacuacggcggaaagaagcagcucugguucccuucuaauuacgucgaggaaauggucaacggcggaggaggcucgggcggcggaggcuccgauccagaagaggacacuguagagucaguggucagcccaccggaauugccgccucggaacauuccuuuaacagcaucauccugugaggcaaaggaggugccuuucagcaacgagaacccacgcgcuacugagacauccagaccaucacuauccgagacucuguuccaaaggcuacagagcauggauacuucuggucugccugaagaacaucucaaggcaauacaggauuaccuaaguacccagcuggcucaggacuccgaauucgugaagaccggcucuagcucucuuccgcaccucaagaagcucacgacgcugcugugcaaggagcucuacggugaagugauccggacgcucccuucccuagagagucuacagagauuguucgaccagcagcuguccccuggauugcguccacguccgcaagugccaggcgaggccaacccuaucaauauggugaguaagcugucacagcugacaagcuugcuaagcagcaucgaggacaaggugaaggcccugcuccacgaggguccggaaaguccacauaggccuagccugaucccaccagugaccuucgaagucaaggcugagagucugggcaucccacagaagaugcagcucaaggucgacguggaaucuggcaagcugaucaucaagaaguccaaggacggcucugaggauaaguucuacucucacaagaagaucuugcaguuaauaaagagucagaaguuccucaacaagcuggugaucuugguggaaacagagaaggagaagauccugcgcaaggaguacguguucgccgauucaaagaagagagaaggcuucugccaacugcuccaacagaugaagaauaagcacuccgaacagccugagccugacaugauuacaaucuucaucggcaccuggaauauggguaacgcaccaccuccuaagaagauuaccaguugguuccucagcaagggccagggaaagacacgggacgacucugcagacuauauuccacacgacaucuacgucauuggaacucaggaggacccacugucggagaaggaguggcuagagauccucaagcauagccuucaggagauuacauccguuacuuucaagacaguggccauccacacauuguggaauauuaggauugucguccucgcuaagccagaacacgagaaccgaaucagucacauuugcaccgauaacgugaagacgggcauagcgaacaccuugggaaauaagggagccgugggcguguccuucauguucaacgguacaagucuggguuucgugaauucgcaccugacguccggcagcgagaagaagcuucggcggaaccagaacuacaugaauauucugcgauuccuagcgcugggagauaagaaguugaguccuuucaauauuacacacagauucacgcaucuguucugguucggagaucuuaacuaucgcgucgaccugccuacgugggaagccgaaacuauuauucagaagaucaagcaacagcaauacgccgacuuacugucucacgaucaguugcuaaccgaaaggcgcgagcagaaggucuuccuccacuucgaagaggaagagauuacuuucgccccuaccuauagguucgaacgccugacacgcgacaaguacgcauacacgaagcagaaggcuaccggcaugaaguacaauuugccuagcuggugcgauagagugcuguggaagucuuacccgcucguacacguggugugucaguccuacggauccacuagugacaucaugaccuccgaucauucaccgguuuucgcuacuuucgaagcuggcgugacgucccaauucguguccaagaacggaccgggcacgguggauagccaaggccaaaucgaguucuugcgcugcuacgccacauugaagacuaagucgcagaccaaguucuaucucgaauuccacaguaguugucuggaaagcuucguaaagagccaggaaggagagaacgaagaagguuccgagggagaacugguggucaaguucggcgaaacacugccuaagcucaagccaauaaucucagauccggaauaccugcuggaccaacauauccugaucucuaucaaguccucagacagcgacgaaucauacggcgagggcuguauugcccuuagauuggaagccacugaaacccaacugccuaucuacacaccacugacacaucacggcgaguuaacgggacacuuccagggugaaauuaagcuucagaccucgcaaggaaagaccagagagaagcucuacgacuucgucaagacugagcgcgacgaaagcagcggcccaaagacacugaagucuuugacaagucacgacccaaugaagcagugggaagugacuagccgcgcuccuccuugcucgggaaguucgaucacugagauu

SEQ ID NO: 75 - RNA, 1986 nt, Artificial Sequence
  Synthetic TCD41 nt. seq.
augaccgccaucaucaaggagaucgugagcagaaacgagagaagauaccaggaggacggcuucgaccuggaccugaccuacaucuacccuaacaucaucgccaugggcuucccugccgagagacuggagggcguguacagaaacaacaucgacgacguggugagauuccuggacagcaagcacaagaaccacuacaagaucuacaaccugugcgcugaacgccacuacgacaccgccaaguucaacugcagaguggcccaguacccuuucgaggaccacaacccuccucagcuggagcugaucaagccuuucugcgaggaucuugaccaguggcugagcgaggacgacaaccacguggccgccauccacugcaaggccggcaagggcagaaccggcgugaugaucugcgccuaccugcugcacagaggcaaguuccugaaggcccaggaggcccuggacuucuacggcgaggugagaaccagagacaagaagggcgugaccaucccuagccaaaggagauacguguacuacuacucuuaucugcugaagaaccaccuggacuacagaccuguggcccugcuguuccacaagaugauguucgaaaccauaccgauguucagcggcggcaccugcaacccucaguucguggugugccagcugaaggugaagauuuacagcagcaacagcggcccuaccagaagagaggacaaguucauguacuucgaguucccucagccucugccugugugcggcgacaucaagguggaguucuuccacaagcagaacaagaugcuuaagaaggacaagauguuccacuucugggugaacaccuucuucaucccuggcccugaggaaaccagcgaggagguggagaacggcagccugugcgaccaggagaucgacagcaucugcagcaucgagagagccgacaacgacaaggaguaccuggugcugacccugaccaagaacgacuuggacaaggccaacaaggacaaggcaaauagauacuucagcccuaacuucaaggugaagcuguacuucaccaagaccgguggaggaggauccggcgguggcggcagcugguuccacggcaagcugggcgccggcagagacggcagacacauugcugagagacugcugaccgaguacugcaucgaaaccggcgccccugacggcagcuuccuggugagagagagcgaaacuuucgugggcgacuacacccugagcuucuggagaaacggcaaggugcagcacugcagaauccacagcagacaggacgccggcaccccuaaguucuuccugaccgacaaccugguguucgacagccuguacgaccugaucacccacuaccagcaggugccucugcggugcaacgaguucgagaugagacugagcgagccugugccucagaccaacgcccacgagagcaaggagugguaccacgccagccugaccagagcccaggccgagcacaugcugaugagagugccuagagacggcgcauuccucguaagaaagagaaacgagccuaacagcuacgccaucagcuucagagccgagggcaagaucaagcauugcagagugcagcaggagggccagaccgugaugcugggcaacagcgaauucgacucucugguggaucugaucagcuacuacgagaagcacccucuuuaccgcaagaugaagcugagauacccuaucaacgaagaggcccuugagaagaucggcaccgccgagccugacuacggcgcccuguacgagggcagaaacccuggcuucuacguggaggccaacccuaugccuaccuucaagugcgccgugaaggcccuguucgacuacaaggcccagagagaggacgagcugaccuucaucaagucugcgauuauccagaacguggagaagcaagagggcggcugguggagaggcgacuacggcggcaagaagcagcugugguucccuagcaacuacgucgaagagauggugaac

SEQ ID NO: 76 - RNA, 1986 nt, Artificial Sequence
  Synthetic TCD42 nt. seq.
augugguuccacggcaagcugggcgccggcagagacggcagacacaucgccgagagacugcugaccgaguacugcaucgaaaccggcgccccugacggcagcuuccuggugagagagagcgaaacuuucgugggcgacuacacccugagcuucuggagaaacggcaaggugcagcacugcagaauccacagcagacaggacgccggcaccccuaaguucuuccugaccgacaaccugguguucgacagccuguacgaccugaucacccacuaccagcaggugccucugcgcugcaacgaguucgagaugagacugagcgagccugugccucagaccaacgcccacgagagcaaggagugguaccacgccagccugaccagagcccaggccgagcacaugcugaugagagugccuagagacggcgcuuuccugguuagaaagagaaacgagccuaacagcuacgccaucagcuucagagccgagggcaagaucaagcacuguagagugcagcaggagggccagaccgugaugcugggcaacagcgaauucgauucccugguggaccuuaucagcuacuacgagaagcacccucuguacagaaagaugaagcugagauacccuaucaacgaggaggcccuggagaagaucggcaccgccgagccugacuacggcgcccuguacgagggcagaaacccuggcuucuacguggaggccaacccuaugccuaccuucaagugcgccgugaaggcccuguucgacuacaaggcccagagagaggacgagcugaccuucaucaagagcgccaucauccagaacguggagaagcaggaaggcggcugguggagaggcgauuacggaggcaagaagcagcugugguucccuagcaauuacgucgaggagauggugaacggaggagguggcucugguggaggcgguagcacagccaucaucaaggagaucgugagcagaaacgagagaagauaccaggaggacggcuucgaccuggaccugaccuacaucuacccuaacaucaucgccaugggcuucccugcggaacggcuggagggcguuuaucgcaacaacaucgacgacguggugagauuccuggacagcaagcacaagaaccacuacaagaucuacaaccugugugcggagcgccacuacgacaccgccaaguucaauugccggguggcccaguacccuuucgaggaccacaacccuccucagcuggagcugaucaagccuuucugcgaggaucuggaccaguggcugagcgaggacgacaaccacguggccgccauccacugcaaggccggcaagggcagaaccggcgugaugaucugcgccuaccugcugcacagaggcaaguuccugaaggcccaggaggccuuggacuucuacggcgaggugagaaccagagacaagaagggcgugaccaucccuagccagcggagauacguguacuacuacucuuaccuucugaagaaccaccuggacuacagaccuguggcccugcuguuccacaagaugauguucgaaaccauaccaauguucucuggaggcaccugcaacccucaguucguggugugccagcugaaggugaagauuuauagcagcaacagcggcccuaccagaagagaggacaaguucauguacuucgaguucccucagccucugccaguuuguggcgacaucaagguggaguucuuccacaagcagaacaagauguugaagaaggauaagauguuccacuucugggugaacaccuucuucaucccuggcccugaggaaaccagcgaggagguggagaacggcagccugugcgaccaggagaucgacagcaucugcagcaucgagagagccgacaacgacaaggaguaccuggugcugacccugaccaagaacgacuuggacaaggccaacaaggauaaggccaauagauacuucagcccuaacuucaaggugaagcuguacuucaccaagacg

SEQ ID NO: 77 - RNA, 1125 nt, Artificial Sequence
  Synthetic TCD43 nt. seq.
augaccgccaucaucaaggagaucgugagcagaaacgagagaagauaccaggaggacggcuucgaccuggaccugaccuacaucuacccuaacaucaucgccaugggcuucccugccgagagacuggagggcguguacagaaacaacaucgacgacguggugagauuccuggacagcaagcacaagaaccacuacaagaucuacaaccugugcgccgagagacacuacgacaccgccaaguucaacugcagaguggcccaguacccuuucgaggaccacaacccuccucagcuggagcugaucaagccuuucugcgaggaccuggaccaguggcugagcgaggacgacaaccacguggccgccauccacugcaaggccggcaagggcagaaccggcgugaugaucugcgccuaccugcugcacagaggcaaguuccugaaggcccaggaggcccuggacuucuacggcgaggugagaaccagagacaagaagggcgugaccaucccuagccagagaagauacguguacuacuacagcuaccugcugaagaaccaccuggacuacagaccuguggcccugcuguuccacaagaugauguucgagacaaucccuauguucagcggcggcaccugcaacccucaguucguggugugccagcugaaggugaagaucuacagcagcaacagcggcccuaccagaagagaggacaaguucauguacuucgaguucccucagccucugccugugugcggcgacaucaagguggaguucuuccacaagcagaacaagaugcugaagaaggacaagauguuccacuucugggugaacaccuucuucaucccuggcccugaggagacaagcgaggagguggagaacggcagccugugcgaccaggagaucgacagcaucugcagcaucgagagagccgacaacgacaaggaguaccuggugcugacccugaccaagaacgaccuggacaaggccaacaaggauaaggccaacagauacuucagcccuaacuucaaggugaagcuguacuucaccaagaccggcggcggcagcuacagacugaagaagaucagcaaggaggagaagaccccuggcugcgugaagaucaagaagugc

SEQ ID NO: 78 - RNA, 963 nt, Artificial Sequence
  Synthetic TCD44 nt. seq.
auggaccagagagagauccugcagaaguuccuggacgaggcccagagcaagaagaucaccaaggaggaguucgccaacgaguuccugaagcugaagagacagagcaccaaguacaaggccgacaagaccuacccuaccaccguggccgagaagccuaagaacaucaagaagaacagauacaaggacauccugccuuacgacuacagcagaguggagcugagccugaucaccagcgacgaggacagcagcuacaucaacgccaacuucaucaagggcguguacggcccuaaggccuacaucgccacccagggcccucugagcaccacccugcuggacuucuggagaaugaucugggaguacagcgugcugaucaucgugauggccugcauggaguacgagaugggcaagaagaagugcgagagauacugggccgagccuggcgagaugcagcuggaguucggcccuuucagcgugagcugcgaggccgagaagagaaagagcgacuacaucaucagaacccugaaggugaaguucaacagcgagacaagaaccaucuaccaguuccacuacaagaacuggccugaccacgacgugccuagcagcaucgacccuauccuggagcugaucugggacgugcgcugcuaccaggaggacgacagcgugccuaucugcauccacugcagcgccggcugcggcagaaccggcgugaucugcgccaucgacuacaccuggaugcugcugaaggacggcaucaucccugagaacuucagcguguucagccugaucagagagaugagaacccagcgaccuagccuggugcagacccaggagcaguacgagcugguguacaacgccgugcuggagcuguucgguggcggcggcucuggcgguggaggcagcuacagacugaagaagaucagcaaggaggagaagaccccuggcugcgugaagaucaagaagugc

SEQ ID NO: 79 - RNA, 963 nt, Artificial Sequence
  Synthetic TCD45 nt. seq.
auggaccagagagagauccugcagaaguuccuggacgaggcccagagcaagaagaucaccaaggaggaguucgccaacgaguuccugaagcugaagagacaggccaccaaguacaaggccgacaagaccuacccuaccaccguggccgagaagccuaagaacaucaagaagaacagauacaaggacauccugccuuacgacuacagcagaguggagcugagccugaucaccagcgacgaggacagcagcuacaucaacgccaacuucaucaagggcguguacggcccuaaggccuacaucgccacccagggcccucugagcaccacccugcuggacuucuggagaaugaucugggaguacagcgugcugaucaucgugauggccugcauggaguacgagaugggcaagaagaagugcgagagauacugggccgagccuggcgagaugcagcuggaguucggcccuuucagcgugagcugcgaggccgagaagagaaagagcgacuacaucaucagaacccugaaggugaaguucaacagcgaaacaagaaccaucuaccaguuccacuacaagaacuggccugaccacgacgugccuagcagcaucgacccuauccuggagcugaucugggacgugcguugcuaccaggaggacgacagcgugccuaucugcauccacugcagcgccggcugcggcagaaccggcgugaucugcgccaucgacuacaccuggaugcugcugaaggacggcaucaucccugagaacuucagcguguucagccugaucagagagaugagaacccaacggccuagccuggugcagacccaggagcaguacgagcugguguacaacgccgugcuggagcuguucggcggugguggcucuggcggcggaggcuccuacagacugaagaagaucagcaaggaggagaagaccccuggcugcgugaagaucaagaagugc

SEQ ID NO: 80 - RNA, 1074 nt, Artificial Sequence
  Synthetic TCD46 nt. seq.
augggcugcggcugcagcagccacccugaggacgacuggauggagaacaucgacgugugcgagaacugccacuacccuaucgugccucuggacggcaagggcacccugcugaucagaaacggcagcgaggugcgggauccucuggugaccuacgagggcagcaacccuccugccagcccucugcaggacaaccuggugaucgcccugcacagcuacggcgguggaggcucugguggaggugguucuuucgccaacgaguuccugaagcugaagagacaggccaccaaguacaaggccgacaagaccuacccuaccaccguggccgagaagccuaagaacaucaagaagaacagauacaaggacauccugccuuacgacuacagcagaguggagcugagccugaucaccagcgacgaggacagcagcuacaucaacgccaacuucaucaagggcguguacggcccuaaggccuacaucgccacccagggcccucugagcaccacccugcuggacuucuggagaaugaucugggaguacagcgugcugaucaucgugauggccugcauggaguacgagaugggcaagaagaagugcgagagauacugggccgagccuggcgagaugcagcuggaguucggcccuuucagcgugagcugcgaggccgagaagagaaagagcgacuacaucaucagaacccugaaggugaaguucaacagcgaaacgagaaccaucuaccaguuccacuacaagaacuggccugaccacgacgugccuagcagcaucgacccuauccuggagcugaucugggacgugaggugcuaccaggaggacgacagcgugccuaucugcauccacugcagcgccggcugcggcagaaccggcgugaucugcgccaucgacuacaccuggaugcugcugaaggacggcaucaucccugagaacuucagcguguucagccugaucagagagaugagaacccagcgcccuagccuggugcagacccaggagcaguacgagcugguguacaacgccgugcuggagcugggaggcgguggcucugguaagccuagcacc

SEQ ID NO: 81 - protein, 609 aa, Artificial Sequence
  Synthetic TCD1 a.a. seq.
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 82 - protein, 609 aa, Artificial Sequence
  Synthetic TCD2 a.a. seq.
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGATPEPARITSPDKPRPMPMDTSVAESPASDPEELKDKKLFLKRDNLFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 83 - protein, 536 aa, Artificial Sequence
  Synthetic TCD3 a.a. seq.
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACGGGGSGGGGSFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 84 - protein, 496 aa, Artificial Sequence
  Synthetic TCD4 a.a. seq.
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCGGGGSGGGGSGGGGSGGGGSWYHSSLTREEAERKLYSGAQTDGKFLLRPRKEQGTYALSLIYGKTVYHYLISQDKAGKYCIPEGTKFDTLWQLVEYLKLKADGLIYCLKEACGGGGSGGGGSFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 85 - protein, 380 aa, Artificial Sequence
  Synthetic TCD5 a.a. seq.
WFFGKIPRAKAEEMLSKQRHDGAFLIRESESAPGDFSLSVKFGNDVQHFKVLRDGAGKYFLWVVKFNSLNELVDYHRSTSVSRNQQIFLRDIEGGGGSGGGGSGGGGSFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 86 - protein, 383 aa, Artificial Sequence
  Synthetic TCD6 a.a. seq.
MWYSGRISRQLAEEILMKRNHLGAFLIRESESSPGEFSVSVNYGDQVQHFKVLREASGKYFLWEEKFNSLNELVDFYRTTTIAKKRQIFLRDEEPLGGGGSGGGGSGGGGSFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 87 - protein, 531 aa, Artificial Sequence
  Synthetic TCD7 a.a. seq.
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETGGGGSGGGGSGGGGSFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 88 - protein, 268 aa, Artificial Sequence
  Synthetic TCD8 a.a. seq.
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGVTYAQLLPDSTPATSTAAPSAPALSTPGIRDSAFSMESIDDVTYAQLPESGESAEASLDGSREVTYAQLSQELHPGAAKTEPAALSSQEAEEVEEEGAPDYENLQELN

SEQ ID NO: 89 - protein, 268 aa, Artificial Sequence
  Synthetic TCD9 a.a. seq.
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGITYAAVLPDSTPATSTAAPSAPALSTPGIRDSAFSMESIDDITYAAVPESGESAEASLDGSREITYAAVSQELHPGAAKTEPAALSSQEAEEVEEEGAPDYENLQELN

SEQ ID NO: 90 - protein, 363 aa, Artificial Sequence
  Synthetic TCD10 a.a. seq.
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGALVVLPDSTPATSTAAPSAPALSTPGIRDSAFSMESIDDAVNVPESGESAEASLDGSREAVNVSQELHPGAAKTEPAALSSQEAEEVEEEGAPDYENLQELNHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 91 - protein, 544 aa, Artificial Sequence
  Synthetic TCD11 a.a. seq.
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGALVVLPDSTPATSTAAPSAPALSTPGIRDSAFSMESIDDAVNVPESGESAEASLDGSREAVNVSQELHPGAAKTEPAALSSQEAEEVEEEGAPDYENLQELNGGGGSGGGGSFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 92 - protein, 327 aa, Artificial Sequence
  Synthetic TCD12 a.a. seq.
MEEAILVPCVLGLLLLPILAMLMALCVHCHRLPGSYDSGFGGGGSGGGGSGGGGSFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 93 - protein, 139 aa, Artificial Sequence
  Synthetic TCD13 a.a. seq.
MEEAILVPCVLGLLLLPILAMLMALCVHCHRLPGSYDSHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 94 - protein, 483 aa, Artificial Sequence
  Synthetic TCD14 a.a. seq.
MEEAILVPCVLGLLLLPILAMLMALCVHCHRLPMSAIQAAWPSGTECIAKYNFHGTAEQDLPFCKGDVLTIVAVTKDPNAYKAKNKVGREGIIPANYVQKREGVKAGTKLSLMPWFHGKITREQAERLLYPPETGLFLVKESTNYPGDYTLCVSCDGKVEHYRIMYHASKLSIDEEVYFENLMQLVAHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 95 - protein, 497 aa, Artificial Sequence
  Synthetic TCD15 a.a. seq.
MGPAGSLLGSGQMQITLWGSLAAVAIFFVITFLIFLCSSCDREKKPRMSAIQAAWPSGTECIAKYNFHGTAEQDLPFCKGDVLTIVAVTKDPNAYKAKNKVGREGIIPANYVQKREGVKAGTKLSLMPWFHGKITREQAERLLYPPETGLFLVKESTNYPGDYTLCVSCDGKVEHYRIMYHASKLSIDEEVYFENLMQLVAHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 96 - protein, 500 aa, Artificial Sequence
  Synthetic TCD16 a.a. seq.
MGCGCSSHPEDDWMENIDVCENCHYPIVPLDGKGTLLIRNGSEVRDPLVTMSAIQAAWPSGTECIAKYNFHGTAEQDLPFCKGDVLTIVAVTKDPNAYKAKNKVGREGIIPANYVQKREGVKAGTKLSLMPWFHGKITREQAERLLYPPETGLFLVKESTNYPGDYTLCVSCDGKVEHYRIMYHASKLSIDEEVYFENLMQLVAHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 97 - protein, 500 aa, Artificial Sequence
  Synthetic TCD17 a.a. seq.
MGCVQCKDKEATKLTEERDGSLNQSSGYRYGTDPTPQHYPSFGVTSIPNYMSAIQAAWPSGTECIAKYNFHGTAEQDLPFCKGDVLTIVAVTKDPNAYKAKNKVGREGIIPANYVQKREGVKAGTKLSLMPWFHGKITREQAERLLYPPETGLFLVKESTNYPGDYTLCVSCDGKVEHYRIMYHASKLSIDEEVYFENLMQLVAHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 98 - protein, 460 aa, Artificial Sequence
  Synthetic TCD18 a.a. seq.
MGSNKSKPKDMSAIQAAWPSGTECIAKYNFHGTAEQDLPFCKGDVLTIVAVTKDPNAYKAKNKVGREGIIPANYVQKREGVKAGTKLSLMPWFHGKITREQAERLLYPPETGLFLVKESTNYPGDYTLCVSCDGKVEHYRIMYHASKLSIDEEVYFENLMQLVAHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 99 - protein, 288 aa, Artificial Sequence
  Synthetic TCD19 a.a. seq.
MEEAILVPCVLGLLLLPILAMLMALCVHCHRLPLKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELH

SEQ ID NO: 100 - protein, 302 aa, Artificial Sequence
  Synthetic TCD20 a.a. seq.
MGPAGSLLGSGQMQITLWGSLAAVAIFFVITFLIFLCSSCDREKKPRLKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELH

SEQ ID NO: 101 - protein, 305 aa, Artificial Sequence
  Synthetic TCD21 a.a. seq.
MGCGCSSHPEDDWMENIDVCENCHYPIVPLDGKGTLLIRNGSEVRDPLVTLKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELH

SEQ ID NO: 102 - protein, 305 aa, Artificial Sequence
  Synthetic TCD22 a.a. seq.
MGCVQCKDKEATKLTEERDGSLNQSSGYRYGTDPTPQHYPSFGVTSIPNYLKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELH

SEQ ID NO: 103 - protein, 265 aa, Artificial Sequence
  Synthetic TCD23 a.a. seq.
MGSNKSKPKDLKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELH

SEQ ID NO: 104 - protein, 305 aa, Artificial Sequence
  Synthetic TCD24 a.a. seq.
MEEAILVPCVLGLLLLPILAMLMALCVHCHRLPFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 105 - protein, 319 aa, Artificial Sequence
  Synthetic TCD25 a.a. seq.
MGPAGSLLGSGQMQITLWGSLAAVAIFFVITFLIFLCSSCDREKKPRFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 106 - protein, 322 aa, Artificial Sequence
  Synthetic TCD26 a.a. seq.
MGCGCSSHPEDDWMENIDVCENCHYPIVPLDGKGTLLIRNGSEVRDPLVTFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 107 - protein, 322 aa, Artificial Sequence
  Synthetic TCD27 a.a. seq.
MGCVQCKDKEATKLTEERDGSLNQSSGYRYGTDPTPQHYPSFGVTSIPNYFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 108 - protein, 282 aa, Artificial Sequence
  Synthetic TCD28 a.a. seq.
MGSNKSKPKDFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 109 - protein, 547 aa, Artificial Sequence
  Synthetic TCD29 a.a. seq.
MEEAILVPCVLGLLLLPILAMLMALCVHCHRLPVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 110 - protein, 561 aa, Artificial Sequence
  Synthetic TCD30 a.a. seq.
MGPAGSLLGSGQMQITLWGSLAAVAIFFVITFLIFLCSSCDREKKPRVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 111 - protein, 564 aa, Artificial Sequence
  Synthetic TCD31 a.a. seq.
MGCGCSSHPEDDWMENIDVCENCHYPIVPLDGKGTLLIRNGSEVRDPLVTVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 112 - protein, 564 aa, Artificial Sequence
  Synthetic TCD32 a.a. seq.
MGCVQCKDKEATKLTEERDGSLNQSSGYRYGTDPTPQHYPSFGVTSIPNYVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 113 - protein, 524 aa, Artificial Sequence
  Synthetic TCD33 a.a. seq.
MGSNKSKPKDVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 114 - protein, 79 aa, Artificial Sequence
  Synthetic TCD34 a.a. seq.
MEADALSPVGLGLLLLPFLVTLLAALCVRCRELPVSYDAVSLSKMLKKRSPLTTGVYVKMPPTEPECEKQFQPYFIPIN

SEQ ID NO: 115 - protein, 87 aa, Artificial Sequence
  Synthetic TCD35 a.a. seq.
MEEAILVPCVLGLLLLPILAMLMALCVHCHRLPGSYDSGFGGGGSTAVSLSKMLKKRSPLTTGVYVKMPPTEPECEKQFQPYFIPIN

SEQ ID NO: 116 - protein, 325 aa, Artificial Sequence
  Synthetic TCD36 a.a. seq.
MEEAILVPCVLGLLLLPILAMLMALCVHCHRLPGSYDSGFGGGGSMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGGKPST

SEQ ID NO: 117 - protein, 892 aa, Artificial Sequence
  Synthetic TCD37 a.a. seq.
MSAEGYQYRALYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQPAPALPPKPPKPTTVANNGMNNNMSLQDAEWYWGDISREEVNEKLRDTADGTFLVRDASTKMHGDYTLTLRKGGNNKLIKIFHRDGKYGFSDPLTFSSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRRGGGGSGGGGSTAIIKEIVSRNERRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEEVENGSLCDQEIDSICSIERADNDKEYLVLTLTKNDLDKANKDKANRYFSPNFKVKLYFTKT

SEQ ID NO: 118 - protein, 1342 aa, Artificial Sequence
  Synthetic TCD38 a.a. seq.
MSAEGYQYRALYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQPAPALPPKPPKPTTVANNGMNNNMSLQDAEWYWGDISREEVNEKLRDTADGTFLVRDASTKMHGDYTLTLRKGGNNKLIKIFHRDGKYGFSDPLTFSSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRRGGGGSGGGGSDPEEDTVESVVSPPELPPRNIPLTASSCEAKEVPFSNENPRATETSRPSLSETLFQRLQSMDTSGLPEEHLKAIQDYLSTQLAQDSEFVKTGSSSLPHLKKLTTLLCKELYGEVIRTLPSLESLQRLFDQQLSPGLRPRPQVPGEANPINMVSKLSQLTSLLSSIEDKVKALLHEGPESPHRPSLIPPVTFEVKAESLGIPQKMQLKVDVESGKLIIKKSKDGSEDKFYSHKKILQLIKSQKFLNKLVILVETEKEKILRKEYVFADSKKREGFCQLLQQMKNKHSEQPEPDMITIFIGTWNMGNAPPPKKITSWFLSKGQGKTRDDSADYIPHDIYVIGTQEDPLSEKEWLEILKHSLQEITSVTFKTVAIHTLWNIRIVVLAKPEHENRISHICTDNVKTGIANTLGNKGAVGVSFMFNGTSLGFVNSHLTSGSEKKLRRNQNYMNILRFLALGDKKLSPFNITHRFTHLFWFGDLNYRVDLPTWEAETIIQKIKQQQYADLLSHDQLLTERREQKVFLHFEEEEITFAPTYRFERLTRDKYAYTKQKATGMKYNLPSWCDRVLWKSYPLVHVVCQSYGSTSDIMTSDHSPVFATFEAGVTSQFVSKNGPGTVDSQGQIEFLRCYATLKTKSQTKFYLEFHSSCLESFVKSQEGENEEGSEGELVVKFGETLPKLKPIISDPEYLLDQHILISIKSSDSDESYGEGCIALRLEATETQLPIYTPLTHHGELTGHFQGEIKLQTSQGKTREKLYDFVKTERDESSGPKTLKSLTSHDPMKQWEVTSRAPPCSGSSITE

SEQ ID NO: 119 - protein, 1113 aa, Artificial Sequence
  Synthetic TCD39 a.a. seq.
MDPEEDTVESVVSPPELPPRNIPLTASSCEAKEVPFSNENPRATETSRPSLSETLFQRLQSMDTSGLPEEHLKAIQDYLSTQLAQDSEFVKTGSSSLPHLKKLTTLLCKELYGEVIRTLPSLESLQRLFDQQLSPGLRPRPQVPGEANPINMVSKLSQLTSLLSSIEDKVKALLHEGPESPHRPSLIPPVTFEVKAESLGIPQKMQLKVDVESGKLIIKKSKDGSEDKFYSHKKILQLIKSQKFLNKLVILVETEKEKILRKEYVFADSKKREGFCQLLQQMKNKHSEQPEPDMITIFIGTWNMGNAPPPKKITSWFLSKGQGKTRDDSADYIPHDIYVIGTQEDPLSEKEWLEILKHSLQEITSVTFKTVAIHTLWNIRIVVLAKPEHENRISHICTDNVKTGIANTLGNKGAVGVSFMFNGTSLGFVNSHLTSGSEKKLRRNQNYMNILRFLALGDKKLSPFNITHRFTHLFWFGDLNYRVDLPTWEAETIIQKIKQQQYADLLSHDQLLTERREQKVFLHFEEEEITFAPTYRFERLTRDKYAYTKQKATGMKYNLPSWCDRVLWKSYPLVHVVCQSYGSTSDIMTSDHSPVFATFEAGVTSQFVSKNGPGTVDSQGQIEFLRCYATLKTKSQTKFYLEFHSSCLESFVKSQEGENEEGSEGELVVKFGETLPKLKPIISDPEYLLDQHILISIKSSDSDESYGEGCIALRLEATETQLPIYTPLTHHGELTGHFQGEIKLQTSQGKTREKLYDFVKTERDESSGPKTLKSLTSHDPMKQWEVTSRAPPCSGSSITEIGGGGSGGGGSWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVN

SEQ ID NO: 120 - protein, 1113 aa, Artificial Sequence
  Synthetic TCD40 a.a. seq.
MWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVNGGGGSGGGGSDPEEDTVESVVSPPELPPRNIPLTASSCEAKEVPFSNENPRATETSRPSLSETLFQRLQSMDTSGLPEEHLKAIQDYLSTQLAQDSEFVKTGSSSLPHLKKLTTLLCKELYGEVIRTLPSLESLQRLFDQQLSPGLRPRPQVPGEANPINMVSKLSQLTSLLSSIEDKVKALLHEGPESPHRPSLIPPVTFEVKAESLGIPQKMQLKVDVESGKLIIKKSKDGSEDKFYSHKKILQLIKSQKFLNKLVILVETEKEKILRKEYVFADSKKREGFCQLLQQMKNKHSEQPEPDMITIFIGTWNMGNAPPPKKITSWFLSKGQGKTRDDSADYIPHDIYVIGTQEDPLSEKEWLEILKHSLQEITSVTFKTVAIHTLWNIRIVVLAKPEHENRISHICTDNVKTGIANTLGNKGAVGVSFMFNGTSLGFVNSHLTSGSEKKLRRNQNYMNILRFLALGDKKLSPFNITHRFTHLFWFGDLNYRVDLPTWEAETIIQKIKQQQYADLLSHDQLLTERREQKVFLHFEEEEITFAPTYRFERLTRDKYAYTKQKATGMKYNLPSWCDRVLWKSYPLVHVVCQSYGSTSDIMTSDHSPVFATFEAGVTSQFVSKNGPGTVDSQGQIEFLRCYATLKTKSQTKFYLEFHSSCLESFVKSQEGENEEGSEGELVVKFGETLPKLKPIISDPEYLLDQHILISIKSSDSDESYGEGCIALRLEATETQLPIYTPLTHHGELTGHFQGEIKLQTSQGKTREKLYDFVKTERDESSGPKTLKSLTSHDPMKQWEVTSRAPPCSGSSITEI

SEQ ID NO: 121 - protein, 662 aa, Artificial Sequence
  Synthetic TCD41 a.a. seq.
MTAIIKEIVSRNERRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEEVENGSLCDQEIDSICSIERADNDKEYLVLTLTKNDLDKANKDKANRYFSPNFKVKLYFTKTGGGGSGGGGSWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVN

SEQ ID NO: 122 - protein, 662 aa, Artificial Sequence
  Synthetic TCD42 a.a. seq.
MWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVNGGGGSGGGGSTAIIKEIVSRNERRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEEVENGSLCDQEIDSICSIERADNDKEYLVLTLTKNDLDKANKDKANRYFSPNFKVKLYFTKT

SEQ ID NO: 123 - protein, 375 aa, Artificial Sequence
  Synthetic TCD43 a.a. seq.
MTAIIKEIVSRNERRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEEVENGSLCDQEIDSICSIERADNDKEYLVLTLTKNDLDKANKDKANRYFSPNFKVKLYFTKTGGGSYRLKKISKEEKTPGCVKIKKC

SEQ ID NO: 124 - protein, 321 aa, Artificial Sequence
  Synthetic TCD44 a.a. seq.
MDQREILQKFLDEAQSKKITKEEFANEFLKLKRQSTKYKADKTYPTTVAEKPKNIKKNRYKDILPYDYSRVELSLITSDEDSSYINANFIKGVYGPKAYIATQGPLSTTLLDFWRMIWEYSVLIIVMACMEYEMGKKKCERYWAEPGEMQLEFGPFSVSCEAEKRKSDYIIRTLKVKFNSETRTIYQFHYKNWPDHDVPSSIDPILELIWDVRCYQEDDSVPICIHCSAGCGRTGVICAIDYTWMLLKDGIIPENFSVFSLIREMRTQRPSLVQTQEQYELVYNAVLELFGGGGSGGGGSYRLKKISKEEKTPGCVKIKKC

SEQ ID NO: 125 - protein, 321 aa, Artificial Sequence
  Synthetic TCD45 a.a. seq.
MDQREILQKFLDEAQSKKITKEEFANEFLKLKRQATKYKADKTYPTTVAEKPKNIKKNRYKDILPYDYSRVELSLITSDEDSSYINANFIKGVYGPKAYIATQGPLSTTLLDFWRMIWEYSVLIIVMACMEYEMGKKKCERYWAEPGEMQLEFGPFSVSCEAEKRKSDYIIRTLKVKFNSETRTIYQFHYKNWPDHDVPSSIDPILELIWDVRCYQEDDSVPICIHCSAGCGRTGVICAIDYTWMLLKDGIIPENFSVFSLIREMRTQRPSLVQTQEQYELVYNAVLELFGGGGSGGGGSYRLKKISKEEKTPGCVKIKKC

SEQ ID NO: 126 - protein, 358 aa, Artificial Sequence
  Synthetic TCD46 a.a. seq.
MGCGCSSHPEDDWMENIDVCENCHYPIVPLDGKGTLLIRNGSEVRDPLVTYEGSNPPASPLQDNLVIALHSYGGGGSGGGGSFANEFLKLKRQATKYKADKTYPTTVAEKPKNIKKNRYKDILPYDYSRVELSLITSDEDSSYINANFIKGVYGPKAYIATQGPLSTTLLDFWRMIWEYSVLIIVMACMEYEMGKKKCERYWAEPGEMQLEFGPFSVSCEAEKRKSDYIIRTLKVKFNSETRTIYQFHYKNWPDHDVPSSIDPILELIWDVRCYQEDDSVPICIHCSAGCGRTGVICAIDYTWMLLKDGIIPENFSVFSLIREMRTQRPSLVQTQEQYELVYNAVLELGGGGSGKPST

SEQ ID NO: 127 - protein, 226 aa, Artificial Sequence
  Synthetic hCD79a ITAM (Y/A)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLAEGLNLDDCSMAEDISRGLQGTYQDVGSLNIGDVQLEKP

SEQ ID NO: 128 - protein, 176 aa, Artificial Sequence
  Synthetic hCD79a (1-176)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGL

SEQ ID NO: 129 - protein, 229 aa, Artificial Sequence
  Synthetic hCD79b ITAM (Y/A)
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTAEGLDIDQTATAEDIVTLRTGEVKWSVGEHPGQE

SEQ ID NO: 130 - protein, 184 aa, Artificial Sequence
  Synthetic hCD79b (1-184)
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKD

SEQ ID NO: 131 - protein, 313 aa, Artificial Sequence
  Synthetic hCD19 (ecto-TM)
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 132 - protein, 556 aa, Artificial Sequence
  Synthetic hCD19 (Y/A)
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQAGNVLSLPTPTSGLGRAQRWAAGLGGTAPSAGNPSSDVQADGALGSRSPPGVGPEEEEGEGAEEPDSEEDSEFYENDSNLGQDQLSQDGSGAENPEDEPLGPEDEDSFSNAESYENEDEELTQPVARTMDFLSPHGSAWDPSREATSLGSQSAEDMRGILYAAPQLRSIRGQPGPNHEEDADSYENMDNPDGPDPAWGGGGRMGTWSTR

SEQ ID NO: 133 - protein, 313 aa, Artificial Sequence
  Synthetic hCD64 (ecto-TM)
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTI

SEQ ID NO: 134 - protein, 320 aa, Artificial Sequence
  Synthetic mCD64 (ecto-TM)
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYV

SEQ ID NO: 135 - protein, 220 aa, Artificial Sequence
  Synthetic mCD79a ITAM (Y/A)
MPGGLEALRALPLLLFLSYACLGPGCQALRVEGGPPSLTVNLGEEARLTCENNGRNPNITWWFSLQSNITWPPVPLGPGQGTTGQLFFPEVNKNHRGLYWCQVIENNILKRSCGTYLRVRNPVPRPFLDMGEGTKNRIITAEGIILLFCAVVPGTLLLFRKRWQNEKFGVDMPDDYEDENLAEGLNLDDCSMAEDISRGLQGTYQDVGNLHIGDAQLEKP

SEQ ID NO: 136 - protein, 228 aa, Artificial Sequence
  Synthetic mCD79b ITAM (Y/A)
MATLVLSSMPCHWLLFLLLLFSGEPVPAMTSSDLPLNFQGSPCSQIWQHPRFAAKKRSSMVKFHCYTNHSGALTWFRKRGSQQPQELVSEEGRIVQTQNGSVYTLTIQNIQYEDNGIYFCKQKCDSANHNVTDSCGTELLVLGFSTLDQLKRRNTLKDGIILIQTLLIILFIIVPIFLLLDKDDGKAGMEEDHTAEGLNIDQTATAEDIVTLRTGEVKWSVGEHPGQE

SEQ ID NO: 137 - protein, 311 aa, Artificial Sequence
  Synthetic mCD19 (ecto-TM)
MPPPRLLFFLLFLTPMEVRPQKSLLVEVEEGGNVVLPCLPDSSPVSSEKLAWYRGNQSTPFLELSPGSPGLGLHVGSLGILLVIVNVSDHMGGFYLCQKRPPFKDIWQPAWTVNVEDSGEMFRWNASDVRDLDCDLRNRSSGSHRSTSGSQLYVWAKDHPKVWGTKPVCAPRGSSLNQSLINQDLTVAPGSTLWLSCGVPPVPVAKASISWTHVHPRRPNVSLLSLSLGGEHPVREMWVWGSLLLLPQATALDEGTYYCLRGNLTIERHVKVIARSAVWLWLLRTGGWIVPVVTLVYVIFCMVSLVAFLYC

SEQ ID NO: 138 - protein, 547 aa, Artificial Sequence
  Synthetic mCD19 (Y/A)
MPSPLPVSFLLFLTLVGGRPQKSLLVEVEEGGNVVLPCLPDSSPVSSEKLAWYRGNQSTPFLELSPGSPGLGLHVGSLGILLVIVNVSDHMGGFYLCQKRPPFKDIWQPAWTVNVEDSGEMFRWNASDVRDLDCDLRNRSSGSHRSTSGSQLYVWAKDHPKVWGTKPVCAPRGSSLNQSLINQDLTVAPGSTLWLSCGVPPVPVAKASISWTHVHPRRPNVSLLSLSLGGEHPVREMWVWGSLLLLPQATALDEGTYYCLRGNLTIERHVKVIARSAVWLWLLRTGGWIVPVVTLVYVIFCMVSLVAFLYCQRAFILRRKRKRMTDPARRFFKVTPPSGNGTQNQYGNVLSLPTSTSGQAHAQRWAAGLGSVPGSAGNPRIQVQDTGAQSHETGLEEEGEAAEEPDSEEGSEFYENDSNLGQDQVSQDGSGAENPEDEPMGPEEEDSFSNAESYENADEELAQPVGRMMDFLSPHGSAWDPSREASSLGSQSAEDMRGILYAAPQLHSIQSGPSHEEDADSYENMDKSDDLEPAWEGEGHMGTWGTT

SEQ ID NO: 139 - protein, 170 aa, Artificial Sequence
  Synthetic mCD79a (ecto-TM)
MPGGLEALRALPLLLFLSYACLGPGCQALRVEGGPPSLTVNLGEEARLTCENNGRNPNITWWFSLQSNITWPPVPLGPGQGTTGQLFFPEVNKNHRGLYWCQVIENNILKRSCGTYLRVRNPVPRPFLDMGEGTKNRIITAEGIILLFCAVVPGTLLLFRKRWQNEKFGV

SEQ ID NO: 140 - protein, 183 aa, Artificial Sequence
  Synthetic mCD79b (ecto-TM)
MATLVLSSMPCHWLLFLLLLFSGEPVPAMTSSDLPLNFQGSPCSQIWQHPRFAAKKRSSMVKFHCYTNHSGALTWFRKRGSQQPQELVSEEGRIVQTQNGSVYTLTIQNIQYEDNGIYFCKQKCDSANHNVTDSCGTELLVLGFSTLDQLKRRNTLKDGIILIQTLLIILFIIVPIFLLLDKD

SEQ ID NO: 141 - protein, 311 aa, Artificial Sequence
  Synthetic rCD19 (ecto-TM)
MPSPLPVSLLLFLTLVGGRPQNSLLVEVEEGDNVVLSCLRDSSPVSSEKLAWYRGNQSTPFLELSLRSPDLGLHIGPLGILLVIVNVSDHRGGFYLCQKRPSFKDTWQPAWTVNVEDSGELFRWNASDLGDLDCDLGNRSSGSHRSTSGSQLYVWATDHPEVWKTKPVCAPREISLNQSLINQDLTVAPGSTLWLSCGVPPVPVTKGSISWTHVHPKTLNVSLLSLSLGGEHPVREMWVWGSLLLLPQAKASDEGTYYCLQGGLTIKMHVKVIARSAVWLWLLRTGGWIVPVVTLVYVIFCMVSMAAFLYF

SEQ ID NO: 142 - protein, 171 aa, Artificial Sequence
  Synthetic rCD79a (ecto-TM)
MLGGLGVLRTLPLLLLFLSEACLGPGCQALMLERDPPSLTVNLGEEAVLTCKNDGKNPNITWWFSLQSNSTWPPMPLGPGLGPMGKLIFPEVNKSHRGLYWCQVIESKEVKRSCGTYLRVRKQVPRPFLDMGEGTKNRIITAEGIILLFCAVVPGTLLLFRKRWQNEKFGV

SEQ ID NO: 143 - protein, 183 aa, Artificial Sequence
  Synthetic rCD79b (ecto-TM)
MATLVLSPVPCHWLMFLLLLLSGEPVPAMTKSDQPPIFQGSPCSKIWQHPRFAAKKRSSMVKFHCHTDYSGVMTWFRQKGNQRPQELFPEDGHISQTRNGSVYTLTLQNIQYEDNGIYFCQQKCNSTEPDVTDGCGTELLVLGFSTLDQLKRRNTLKDGIIMIQTLLIILFIIVPIFLLLDKD

SEQ ID NO: 144 - protein, 96 aa, Artificial Sequence
  Synthetic hCD22 ITIM
YNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 145 - protein, 272 aa, Artificial Sequence; identical to SEQ ID NO: 21
  Synthetic hSHP1(PTP)

SEQ ID NO: 146 - protein, 70 aa, Artificial Sequence
  Synthetic hCD32b ITIM
VVALIYCRKKRISALPGYPECREMGETLPEKPANPTNPDEADKVGAENTITYSLLMHPDALEEPDDQNRI

SEQ ID NO: 147 - protein, 100 aa, Artificial Sequence
  Synthetic mCD32b ITIM
VSLVYLKKKQVPALPGNPDHREMGETLPEEVGEYRQPSGGSVPVSPGPPSGLEPTSSSPYNPPDLEEAAKTEAENTITYSLLKHPEALDEETEHDYQNHI

SEQ ID NO: 148 - protein, 96 aa, Artificial Sequence
  Synthetic mCD22 ITIM
YNPAMDDTVSYAILRFPESDTHNTGDAGTPATQAPPPNNSDSVTYSVIQKRPMGDYENVNPSCPEDESIHYSELVQFGAGKRPQAKEDVDYVTLKH

SEQ ID NO: 149 - protein, 96 aa, Artificial Sequence
  Synthetic rCD22 ITIM
YNLAMDDTVSYAVLRFPESDTHGAGGARSPATQGPPPNDDDTVTYSVLQKRNMGDYENVSPNCPEDESIHYSELVQFGAGKRPQAKEDVDYVTLKH

SEQ ID NO: 150 - RNA, 966 nt, Artificial Sequence
  Synthetic BCD1 n.t. seq.
augccuggcggcccuggcgugcugcaggcccugccugccaccaucuuccugcuguucuuacucagcgccguguaccugggaccaggcugccaggcccuguggaugcacaaggucccagcuagccugauggugagccugggcgaggacgcccacuuccagugcccucacaacagcagcaacaacgccaacgugaccugguggagagugcugcacggcaacuacaccuggccuccugaguuccugggcccaggugaagauccuaacggcacccugaucauccagaacgugaacaagagccacggcggcaucuacgugugcagagugcaggagggcaacgagagcuaccagcagagcugcggcaccuaccugagagugagacagccuccuccuagaccuuuccuggacaugggagaaggcaccaagaacagaaucaucaccgccgagggcaucauucuccucuucugcgccguggugccugguaccuugcuacuuuucagaaagcgguggcagaacgagaagcugggccuggacgccggcgacgaguacgaggacgagaaccuggcugaaggccugaaccuggacgacugcagcauggccgaggacaucagcagaggccugcagggaacuuaccaggacgugggcagccugaacaucggcgacgugcagcuggagaagccuuacaacccuaugauggaggacggcaucagcuacaccacccugagauucccggagaugaacaucccuagaacaggcgacgccgagagcagcgagaugcagcgaccaccuccugacugcgacgacaccgugaccuacagcgcccugcacaagagacaggugggcgacuacgagaacgucaucccugacuucccagaggacgagggaauccacuacagcgagcugauccaguucggcgugggcgaacggccacaggcccaggagaacguggacuacgugauucugaagcac

SEQ ID NO: 151 - RNA, 816 nt, Artificial Sequence
  Synthetic BCD2 n.t. seq.
augccuggcggcccuggcgugcugcaggcccugccugccaccaucuuccugcuguuccugcugagcgccguguaccugggcccuggcugccaggcccuguggaugcacaaggugccugccagccugauggugagccugggcgaggacgcccacuuccagugcccucacaacagcagcaacaacgccaacgugaccugguggagagugcugcacggcaacuacaccuggccuccugaguucuuaggaccuggcgaggacccuaacggcacccugaucauccagaacgugaacaagagccacggcggcaucuacgugugcagagugcaggagggcaacgagagcuaccagcagagcugcggcaccuaccugagagugagacagccuccuccuagaccuuuccuggacaugggcgagggcaccaagaacagaaucaucaccgccgagggcaucauccugcuguucugcgccguggugccuggcacccugcugcuguucagaaagagguggcagaacgagaagcugggccuguacaacccuaugauggaggacggcaucagcuacaccacccugagauucccugagaugaacaucccuagaaccggcgacgccgagagcagcgagaugcagcgcccuccuccugacugcgacgacaccgugaccuacagcgcccugcacaagagacaggugggcgacuacgagaacgugaucccugacuucccugaggacgagggcauccacuacagcgagcugauccaguucggcgugggcgagcguccucaggcccaggagaacguggacuacgugauccugaagcac

SEQ ID NO: 152 - RNA, 975 nt, Artificial Sequence
  Synthetic BCD3 nt. seq.
auggccagacuggcccugagcccugugccuagccacuggaugguggcccugcuguuacucuugagcgccgagccugugccugccgccagaagcgaggacagauacagaaacccuaagggcagcgccugcagcagaaucuggcagagcccuagauucaucgccagaaagagaggcuucaccgugaagaugcacugcuacaugaacagcgccagcggcaacgugagcuggcuguggaagcaggagauggacgagaacccucagcagcugaagcuggagaagggcagaauggaggagagccagaacgagagccuggccacccugaccauccagggcaucagauucgaggacaacggcaucuacuucugccagcagaagugcaacaacaccagcgagguguaccagggcugcggcaccgagcugagagugaugggcuucagcacccuggcccagcugaagcagagaaacacccugaaggacggcaucaucaugauccagacccugcugaucauccuguucaucaucgugccuaucuuccugcugcuggacaaggacgacagcaaggccggcauggaggaggaccacaccgccgagggccuggacaucgaccagaccgccaccgccgaggacaucgugacccugagaaccggcgaggugaaguggagcgugggcgagcauccaggacaggaguacaacccuaugauggaagacgguaucagcuacaccacccugagauucccugagaugaacaucccuagaaccggcgacgccgagagcagcgagaugcaaagaccuccuccugacugcgacgacaccgugaccuacagcgcccugcacaagagacaggugggcgacuacgagaacgugaucccugacuucccugaggacgagggcauccacuacagcgagcugauccaguucggcgugggcgaacguccucaggcccaggagaacguggacuacgugauccugaagcac

SEQ ID NO: 153 - RNA, 840 nt, Artificial Sequence
  Synthetic BCD4 nt. seq.
auggccagacuggcccugagcccugugccuagccacuggaugguggcccugcugcugcugcugagcgccgagccugugccugccgccagaagcgaggacagauacagaaacccuaagggcagcgccugcagcagaaucuggcagagcccuagauucaucgccagaaagagaggcuucaccgugaagaugcacugcuacaugaacagcgccagcggcaacgugagcuggcuguggaagcaggagauggacgagaacccucagcagcugaagcuggagaagggcagaauggaggagagccagaacgagagccuggccacccugaccauccagggcaucagauucgaggacaacggcaucuacuucugccagcagaagugcaacaacaccagcgagguguaccagggcugcggcaccgagcugagagugaugggcuucagcacccuggcccagcugaagcagagaaacacccugaaggacggcaucaucaugauccagacccugcugaucauccuguucaucaucgugccuaucuuccugcugcuggacaaggacuacaacccuaugauggaggacggcaucagcuacaccacccugagauucccugagaugaacaucccuagaaccggcgacgccgagagcagcgagaugcagagaccuccuccugacugcgacgacaccgugaccuacagcgcccugcacaagagacaggugggcgacuacgagaacgugaucccugacuucccugaggacgagggcauccacuacagcgagcugauccaguucggcgugggcgagagaccucaggcccaggagaacguggacuacgugauccugaagcac

SEQ ID NO: 154 - RNA, 1257 nt, Artificial Sequence
  Synthetic BCD5 nt. seq.
augccuccuccuagacugcuguucuuccugcuguuccugaccccuauggaggugagaccugaggagccucugguggugaagguggaggagggcgacaacgccgugcugcagugccugaagggcaccagcgacggcccuacccagcagcugaccuggagcagagagagcccucugaagccuuuccugaagcugagccugggccugccuggccugggcauccacaugcgaccucuggccaucuggcuguucaucuucaacgugagccagcagaugggcggcuucuaccugugccagccuggcccuccuagcgagaaggccuggcagccuggcuggaccgugaacguggagggcagcggcgagcuguuccgcuggaacgugagcgaccugggaggacugggcuguggccugaagaacagaagcagcgagggcccuagcagcccuagcggcaagcugaugagcccuaagcuguacgugugggccaaggacagaccugagaucugggagggcgagccuccuugccugccuccuagagacagccugaaccagagccugagccaggaccugaccauggccccuggcagcacccuguggcugagcugcggcgugccuccugacagcgugagcagaggcccucugagcuggacccacgugcacccuaagggcccuaagagccugcugagccuggagcugaaggacgacagaccugccagagacaugugggugauggagacaggccugcugcugccuagagccaccgcccaggacgccggcaaguacuacugccacagaggcaaccugaccaugagcuuccaccuggagaucaccgccagaccugugcuguggcacuggcugcugagaaccggcggcuggaaggugagcgccgugacccuggccuaccugaucuucugccugugcagccuggugggcauccugcaccucggcggaggaggaucgggcggagguggcucuuacaacccuaugauggaggacggcaucagcuacaccacccugagauucccugagaugaacaucccuagaaccggcgacgccgagagcagcgagaugcaacgaccuccuccugacugcgacgacaccgugaccuacagcgcccugcacaagagacaggugggcgacuacgagaacgugaucccugacuucccugaggacgagggcauccacuacagcgagcugauccaguucggcgugggcgaaaggccucaggcccaggagaacguggacuacgugauccugaagcac

SEQ ID NO: 155 - RNA, 1800 nt, Artificial Sequence
  Synthetic BCD6 nt. seq.
augccuccuccuagacugcuguucuuccuguuguuccugaccccuauggaggugagaccugaggagccucugguggugaagguggaggagggcgacaacgccgugcugcagugccugaagggcaccagcgacggcccuacccagcagcugaccuggagcagagagagcccucugaagccuuuccugaagcugagccugggccugccuggccugggcauccacaugcgaccucuggccaucuggcuguucaucuucaacgugagccagcagaugggcggcuucuaccugugccagccuggcccuccuagcgagaaggccuggcagccagguuggaccgugaacguggagggcagcggcgagcuguuccgguggaacguaagcgaccugggcggacucgguugcggccugaagaacagaagcagcgagggcccuagcagcccuagcggcaagcugaugagcccuaagcuguacgugugggccaaggacagaccugagaucugggaaggagagccuccuugccugccuccuagggacagccugaaccagagccugagccaggaccugaccauggccccuggaucuacccuguggcugagcugcggcgugccuccugacagcgugagcagaggaccacuuagcuggacccacgugcacccuaagggaccaaagagcuuacugucuuuggagcugaaggacgaccgcccagccagagacaugugggugauggaaacaggccugcugcugccuagagccaccgcucaggacgccggcaaguacuacugccacagaggcaaccugacgaugagcuuccaccuggagaucaccgccagaccugugcuguggcacuggcugcugagaaccggcggcuggaaggugagcgccgugacccuggccuaccugaucuucugccuguguagccucgucggcauacugcaccuggguggcggagguucgggcggcggcggaucaggaggaggcggcuccuucugggaggaguucgaaaguuugcagaagcaagaggugaagaaccugcaccagagacuggagggucagaggccagagaacaagggcaagaacagguacaagaacauccugccuuucgaccacagcagagugauccugcagggccgggacuccaacaucccuggcucagacuacaucaacgccaacuacauaaagaaccagcugcugggcccugacgagaacgccaagaccuacaucgccagccagggcugccuggaggccacugucaacgacuucuggcagauggccuggcaggagaauucccgagugaucgugaugacaaccagagagguggagaagggcagaaacaagugcgugccuuacuggccugagguuggcaugcagagagccuacggcccuuacucugugaccaacuguggugagcacgacaccaccgaguacaaguuacgcacccugcaggucaguccacuggacaacggcgaccugaucagagagauuuggcacuaccaguaccugucuuggccugaccacggagugccauccgagccugguggaguuuuguccuuccuggaccagaucaaucagcggcaggaaucccugccacacgccggcccuaucaucgugcacugcagcgccggcaucggcagaaccgguaccaucauagucaucgacaugcuuauggagaacaucagcaccaagggccuggacugcgauauugauauccagaagaccauccagauggugagagcccagagaagcggcauggugcagaccgaggcccaguauaaguucaucuacguggccaucgcccaguuc

SEQ ID NO: 156 - RNA, 1956 nt, Artificial Sequence
  Synthetic BCD7 nt. seq.
augccuccuccuagacugcuguucuucuugcuuuuccugaccccuauggaggugagaccugaggagccucugguggugaagguggaggagggcgacaacgccgugcugcagugccugaagggcaccagcgacggcccuacccagcagcugaccuggagcagagagagcccucugaagccuuuccugaagcugagccugggccugccuggccugggcauccacaugcggccucuggccaucuggcuguucaucuucaacgugagccagcagaugggcggcuucuaccugugccagccuggcccuccuagcgagaaggccuggcagccuggcuggaccgugaacguggagggcagcggcgagcuguuccguuggaacguuagugaccugggcggacuaggcugcggccugaagaacagaagcagcgagggcccuagcagcccuagcggcaagcugaugagcccuaagcuguacgugugggccaaggacagaccugagaucugggaaggcgagccuccuugccugccaccuagggacagccugaaccagagccugagccaggaccugaccauggccccgggauccacccuguggcugagcugcggcgugccuccugacagcgugagcagaggcccacuuagcuggacccacgugcacccuaagggaccaaagagcuuauuaucccuugagcugaaggacgacaggccagccagagacaugugggugauggaaaccggccugcugcugccuagagccaccgcccaggacgccggcaaguacuacugccacagaggcaaucugacaaugagcuuccaccuggagaucaccgccagaccugugcuguggcacuggcugcugagaaccggcggcuggaaggugagcgccgugacccuggccuaccugaucuucugccugugcucacuggucgguauccugcaccugcagcgcgcucugguccucagaagaaagcgaaagcggaugaccgacccuaccagaagauucuucaaggugaccccaccaccuggcucuggaccucagaaccaggccggcaacgugcucucacugccuaccccaaccuccggucuggguagagcccagaggugggccgcugguuugggcggcaccgccccuagugcagguaauccgucaagcgacgugcaggccgacggcgcccugggcagcagaagcccuccuggagugggcccagaggaggaagagggugaaggcgccgaggagccagauuccgaggaggacucugaguucuacgagaacgacagcaaccugggucaggaccaguugagucaggacgguucuggcgcggagaauccggaggacgagccauugggaccugaggacgaagacucguucagcaacgccgagaguuacgagaacgaagacgaggagcugacccagccuguggccagaaccauggacuuccugagcccucacggcagcgccugggauccaagucgggaggccacaagcuuggguucccaguccgcugaggacaugagaggaauccucuacgccgccccucaguugcggagcauccgcggccagccggguccaaaccacgaggaggacgcagacaguuacgagaacauggacaacccugacggcccggacccugcuuggggcgguggugguagaauggguacuuggaguaccagauacaacccuaugauggaggacgguaucagcuacaccacccugagauucccagaaaugaacaucccucgaaccggagacgcagaaagcuccgaaaugcagcgcccuccuccagauugcgacgacaccgugaccuacagcgcccugcacaagagacaggugggcgacuacgagaacgugaucccugacuucccugaagacgagggaauccacuacagcgagcugauccaguucggcgugggagagaggccucaggcccaggagaacguugacuacgugauucugaagcac

SEQ ID NO: 157 - RNA, 1149 nt, Artificial Sequence
  Synthetic BCD8 nt. seq.
augugguuccugaccacccugcugcugugggugccuguggacggccagguggacaccaccaaggccgugaucacccugcagccuccuugggugagcguguuccaggaggagacugugacccugcacugcgaggugcugcaccugccuggcagcagcagcacccagugguuccucaacggcaccgccacccagaccagcaccccuagcuacagaaucaccagcgccagcgugaacgacagcggcgaguaccggugccagagaggccugagcggcagaagcgacccuauccagcuggagauccacagaggcuggcugcugcugcaggugagcagcagaguguucaccgagggcgagccucuggcccugaggugccacgccuggaaggacaagcugguguacaacgugcuguacuacagaaacggcaaggccuucaaguucuuccacuggaacagcaaccugaccauccugaagaccaacaucagccacaacgguaccuaccacugcagcggcaugggcaagcacagauauacuucugccggcaucagcgugaccgugaaggagcuguucccugccccugugcugaacgcaagugugaccagcccucugcuggagggcaaccuggugacccugagcugcgagacaaagcugcuccugcaaaggccuggccugcagcuguacuucagcuucuacaugggcagcaagacccugagaggcagaaacaccagcagcgaguaccagauccugaccgccagaagagaggacagcggccuguacuggugcgaggccgccaccgaggacggcaacguccugaagagaaguccugagcuggagcuucaggugcugggucugcagcugccuaccccugugugguuccacgugcuguucuaccuggccgugggcaucauguuccuggugaacaccgucuugugggugaccaucgugguggcccugaucuacugcagaaagaagagaaucagcgcccugccgggcuacccugagugcagagagaugggcgagacucugccugagaagccugccaacccuaccaacccugacgaggccgacaaggugggcgccgagaacaccaucaccuacagccugcugaugcacccugacgcccuggaggagccugacgaccagaacagaauc

SEQ ID NO: 158 - RNA, 1260 nt, Artificial Sequence
  Synthetic BCD9 nt. seq.
augauccugaccagcuucggcgacgacauguggcugcugaccacccugcugcugugggugccugugggaggagagguggugaacgccaccaaggccgugaucacccugcagccuccuugggugagcaucuuccagaaggagaacgugacccuguggugcgagggcccucaccugccuggcgacagcagcacccagugguucaucaacggcaccgccgugcagaucagcaccccuagcuacagcaucccugaggccagcuuccaggacagcggcgaguaccgcugccagaucggcagcagcaugccuagcgacccgguccagcugcagauccacaacgacuggcuacugcugcaggccagcagaagagugcugaccgagggcgagccucuggcccugcgaugccacggcuggaagaacaagcugguguacaacgugguguucuacagaaacggcaaguccuuccaauucagcagcgacagcgagguggccauccugaagaccaaccugagccacagcggcaucuaccacugcagcggcaccggcagacacagauacaccagcgccggcgugagcauuaccgugaaggagcuguucaccaccccugugcugagagccucagucucuagcccuuucccggaaggcagccuggucacucugaacugcgagacaaaccugcuacugcagcggccuggccugcaguugcacuucagcuucuacgugggcagcaagauccuggaguaccgaaauacuagcagugaguaccacaucgccagagccgagagagaggacgccggcuucuacuggugugagguugcuaccgaggauuccagcgugcugaagagaagcccugagcuggagcugcaggugcugggcccgcagagcagcgccccugugugguuccacauccuguucuaccugagcgugggcaucauguucagccugaacaccgugcuguacgucguauccuugguauaccugaagaagaagcaggugccugcgcucccaggcaacccugaccacagagagaugggcgaaacccucccugaagagguuggugaauaccgacagccuagcggcggcagcgucccuguaagcccuggcccuccgucuggucuggagccuacaucuaguaguccauacaacccuccugaccuggaggaggccgccaagaccgaggccgagaacaccaucaccuacagccugcugaagcauccagaggcucuggacgaggaaacagagcacgacuaccagaaccacauc

SEQ ID NO: 159 - RNA, 948 nt, Artificial Sequence
  Synthetic BCD10 nt. seq.
augccuggcggccuggaggcccugagagcccugccucugcugcuguuccugagcuacgccugccugggcccuggcugccaggcccugagaguggagggcggcccuccuagccugaccgugaaccugggcgaggaggccagacugaccugcgagaacaacggcagaaacccuaacaucaccuggugguucagccugcagagcaauaucacuuggccuccugugccucuggguccaggccagggcaccaccggccagcuguucuucccugaggugaacaagaaccacagaggccuguacuggugccaggugauugagaauaacauccugaagagaagcugcggcaccuaccugagagugagaaacccugugccuagaccuuuccuggacaugggcgagggcaccaagaacagaaucaucaccgccgagggcaucauccugcuguucugcgccguggugccuggcacccuacuguuauucagaaagagguggcagaacgagaaguucggcguggacaugccugacgacuacgaggacgagaaccuggccgagggccugaaccuggacgacugcagcauggccgaggacaucagccggggucugcagggcaccuaucaggacgugggcaaccugcacaucggcgacgcccagcuggagaagccuuacaacccugccauggacgacaccgucaguuacgccauccugagauucccugaaagcgacacccacaacacuggugacgccggcaccccugccacccaggccccuccuccuaacaacagcgacagcgugaccuacagcgugauccagaagcguccuaugggcgauuacgagaacgugaacccuagcugcccugaagacgaaagcauccacuacagcgagcuggugcaguucggcgccggcaagcgaccucaggccaaggaggacguggacuacgugacccugaagcac

SEQ ID NO: 160 - RNA, 972 nt, Artificial Sequence
  Synthetic BCD11 nt. seq.
auggccacccuggugcugagcagcaugccuugccacuggcugcuguuccugcuacugcuguucagcggcgagccugugccugccaugaccagcagcgaccugccucugaacuuccagggcagcccuugcagccagaucuggcagcacccuagauucgccgccaagaagagaagcagcauggugaaguuccacugcuacaccaaccacagcggcgcccugaccugguucagaaagagaggcagccagcagccucaggagcuggugagcgaggagggcagaaucgugcagacccagaacggcagcguguacacccugaccauccagaacauccaguacgaggacaacggcaucuacuucugcaagcagaagugcgacagcgccaaccacaacgugaccgacagcugcggcaccgagcugcuggugcugggcuucagcacccuggaccagcugaagagaagaaacacccugaaggacggcaucauccugauccagacccugcugaucauccuguucaucaucgugccuaucuuccuacuccuggauaaggacgacggcaaggccggcauggaggaggaccacaccgccgagggccugaacaucgaccagaccgccacggccgaggacaucgugacccugagaaccggcgaggugaaguggagcgugggcgaacauccuggccaggaguacaacccugccauggacgacaccgugagcuacgccauccugagauucccugagagcgacacccacaacaccggcgacgccggcaccccugccacccaggccccuccuccuaacaacagcgacagcgugaccuacagcgugauccagaagaggccuaugggcgacuacgagaacgugaacccuagcugcccugaggacgagagcauccacuacagcgagcuggugcaguucggcgccggcaagcguccucaggccaaggaggacguggacuacguuacacugaagcac

SEQ ID NO: 161 - RNA, 1251 nt, Artificial Sequence
  Synthetic BCD12 nt. seq.
augccuccuccuagacugcuguucuuccugcuguuccugaccccuauggaggugagaccucagaagucucugcugguggagguggaggagggcggcaacguggugcugccuugccugccugacagcagcccugugagcagcgagaagcuggccugguacagaggcaaccagagcaccccuuuccuggagcugagcccuggcagcccuggccugggccugcacgugggcagccugggcauauuacuggugaucgugaacgugagcgaccacaugggcggcuucuaccugugccagaagcguccuccuuucaaggacaucuggcagccugccuggaccguaaacguggaggacagcggcgagauguuccgauggaacgccagcgacgugagggaccuggacugcgaccugagaaacagaagcagcggcagccacagaagcacgaguggaucucagcuguacgugugggccaaggaccacccuaagguguggggcaccaagccugugugcgccccuagaggcagcagccugaaccagagccugaucaaccaggaccugaccguggcuccggguagcacccuguggcugagcugcggcgugccuccugugccuguggccaaggccagcaucagcuggacccacgugcacccuagaagaccuaacguaagccuucugagccugagccugggcggcgaacacccagugagagagaugugggucuggggcuccuuacuccugcugccucaggccaccgcccuggacgagggcaccuacuacugccugaggggaaaccugaccaucgagagacacgugaaggugaucgccagaagcgccguguggcuguggcugcugagaaccggcggcuggaucgucccgguggugacccugguguacgugaucuucugcauggugagccugguggccuuccuguacugcggaggaggcggcuccggaggcggaggcagcuacaacccugccauggacgacaccgugagcuacgccauccugagauucccugagagcgacacccacaacaccggcgacgccggcaccccugccacccaggccccaccuccaaacaacagcgacagcgugaccuacagcgugauucagaagaggccuaugggcgacuacgagaacgugaacccuagcugcccugaggacgagagcauccacuacagcgagcuggugcaguucggcgccggcaagcguccacaggccaaggaggacguggacuacgugacccugaagcac

SEQ ID NO: 162 - RNA, 1929 nt, Artificial Sequence
  Synthetic BCD13 nt. seq.
augccuagcccucugccugugagcuuccugcuguuccugacccuggugggcggcagaccucagaagucgcugcugguggagguggaggagggcggcaacguggugcugccuugccugccugacagcagcccagucucgagcgagaagcuggccugguacagaggcaaccagagcaccccuuuccuggagcugagcccuggaucgccuggucuaggccugcacgugggcagccugggcauacuccuuguaaucgugaacgugagcgaccacaugggcggcuucuaccugugccagaagcgcccuccuuucaaggacauuuggcaaccugcuuggacugucaacguggaggacagcggcgagauguuccgguggaacgccagcgacgugagggaccuggacugcgaccugagaaacagaagcagcggcagccacagauccaccaguggcucccagcuguacgugugggccaaggaccacccuaagguguggggcaccaagccugugugcgccccuagaggcagcagccugaaccagucccugaucaaccaggaccugaccguggcuccgggcucuacccuguggcugagcugcggcgugccuccugugccuguggccaaggccagcaucagcuggacccacgugcacccuagaagaccuaacgugucgcuuuugagccugucacuuggcggcgagcacccugugagagagaugugggucuggggaucccuucuguugcugccucaggccaccgcccuggacgagggcaccuacuacugccugcgcgguaaccugaccaucgagagacacgugaaggugaucgccagaagcgccguguggcuuuggcuucugagaaccggcggcuggaucguuccagucgugacccuaguguacgugaucuucugcaugguuucacuuguggccuuccuguacugccagagagccuucauccugagaagaaagcgcaagagaaugaccgacccagcucgucgauucuucaaggugaccccuccuuccggaaacggcacccagaaccaguacggaaacgugcuaucccugccaaccuccacuagcggacaggcccacgcccagcgaugggccgccggccugggaagcgugccgggaagugccggcaauccucgcauccaggugcaggacaccggagcacagagccacgagacgggcuuagaagaggaaggcgaggccgccgaggaaccugauagcgaagaggguagcgaguucuacgagaacgacagcaaccugggccaggaccaggugagccaggacggcagcggcgccgagaacccugaggacgagccuauggguccugaagaggaggacucuuucagcaacgccgagucuuacgagaacgccgacgaggagcuggcccagccugugggcagaaugauggacuuccugucuccucacggcucagccugggacccuagcagagaagcuaguaguuuaggcagccaaagugcagaagacaugcgugguauucuauacgccgccccucagcugcacagcauccagagcggcccuagucacgaggaagacgccgacaguuacgagaacauggacaagagcgacgaccuggaaccugccugggagggagaaggccauaugggaaccuggggcacuaccuacaacccugccauggacgacaccgugagcuacgccauucucagauucccugagagcgacacccacaauacaggugacgccggcaccccugccacccaggccccuccuccuaacaacagcgacagcgugaccuauuccgucauucagaagcguccaaugggcgacuacgagaacgugaacccuagcuguccugaggacgagagcauccacuacagcgagcuggugcaguucggcgcuggcaagcguccucaggcuaaggaggacguggacuacguaacacugaagcac

SEQ ID NO: 163 - RNA, 798 nt, Artificial Sequence
  Synthetic BCD14 nt. seq.
augccuggcggccuggaggcccugagagcccugccucugcugcuguuccugagcuacgccugccugggcccuggcugccaggcccugagaguggagggcggcccuccuagccugaccgugaaccugggcgaggaggccagacugaccugcgagaacaacggcagaaacccuaacaucaccuggugguucagccugcagagcaauauaacuuggccuccugugccucuugguccaggacagggcaccaccggccagcuguucuucccugaggugaacaagaaccacagaggccuguacuggugccaggugaucgagaacaacauccugaagagaagcugcggcaccuaccugagagugagaaacccugugccuagaccuuuccuggacaugggcgagggcaccaagaacagaaucaucaccgccgagggcaucauccugcuguucugcgccguggugccuggcaccuugcuucuguucagaaagcgauggcagaacgagaaguucggcguguacaacccugccauggacgacaccgugagcuacgccauccugagauucccugagagcgacacccacaacaccggcgacgccggcaccccugccacccaggccccuccuccuaacaacagcgacagcgugaccuacagcgugauccagaagaggccuaugggcgacuacgagaacgugaacccuagcugcccugaggacgagagcauccacuacagcgagcuggugcaguucggcgccggcaagcgcccucaggccaaggaggacguggacuacgugacccugaagcac

SEQ ID NO: 164 - RNA, 837 nt, Artificial Sequence
  Synthetic BCD15 nt. seq.
auggccacccuggugcugagcagcaugccuugccacuggcugcuguuccugcugcugcuguucagcggcgagccugugccugccaugaccagcagcgaccugccucugaacuuccagggcagcccuugcagccagaucuggcagcacccuagauucgccgccaagaagagaagcagcauggugaaguuccacugcuacaccaaccacagcggcgcccugaccugguucagaaagagaggcagccagcagccucaggagcuggugagcgaggagggcagaaucgugcagacccagaacggcagcguguacacccugaccauccagaacauccaguacgaggacaacggcaucuacuucugcaagcagaagugcgacagcgccaaccacaacgugaccgacagcugcggcaccgagcugcuggugcugggcuucagcacccuggaccagcugaagagaagaaacacccugaaggacggcaucauccugauccagacccugcugaucauccuguucaucaucgugccuaucuuccugcugcuggacaaggacuacaacccugccauggacgacaccgugagcuacgccauccugagauucccugagagcgacacccacaacaccggcgacgccggcaccccugccacccaggccccuccuccuaacaacagcgacagcgugaccuacagcgugauccagaagagaccuaugggcgacuacgagaacgugaacccuagcugcccugaggacgagagcauccacuacagcgagcuggugcaguucggcgccggcaagagaccucaggccaaggaggacguggacuacgugacccugaagcac

SEQ ID NO: 165 - RNA, 1251 nt, Artificial Sequence
  Synthetic BCD16 nt. seq.
augccuagcccucugccugugagccugcugcuguuccugacccuggugggcggcagaccucagaacagccugcugguggagguggaggagggcgacaacguggugcugagcugccugagagacagcagcccugugagcagcgagaagcuggccugguacagaggcaaccagagcaccccuuuccuggagcugagccugagaagcccugaccugggccugcacaucggcccucugggcauccugcuggugaucgugaacgugagcgaccacagaggcggcuucuaccugugccagaagcggccuagcuucaaggacaccuggcagccugccuggaccgugaacguggaggacagcggcgagcuguuccgguggaacgccagcgaccugggcgaccuggacugcgaccugggcaacagaagcagcggcagccacagaagcacgaguggaucucagcuguacgugugggccaccgaccacccugagguguggaagaccaagccugugugcgccccuagagagaucagccugaaccagagccugaucaaccaggaccugaccguggccccuggcagcacccuguggcugagcugcggcgugccuccugugccugugaccaagggcagcaucagcuggacccacgugcacccuaagacccugaacgugagcuuacugucccugagccugggcggcgagcacccugugagagagauguggguguggggcucacuccuguugcugccucaggccaaggccagcgacgagggcaccuacuacugccugcagggcggccugaccaucaagaugcacgugaaggugaucgccagaagcgccguguggcuguggcugcugagaaccggcggcuggaucgugccuguggugacccugguguacgugaucuucugcauggugagcauggccgccuuccuguacuucggcggcggcggcucugguggcggaggaagcuacaaccuggccauggacgacaccgugagcuacgccgugcugagauucccugagagcgacacucacggcgccggaggagcaagaagcccugccacccagggcccuccuccuaacgacgacgacaccguuaccuacagcgugcugcagaagagaaacaugggcgacuacgagaacgugagcccuaacugcccugaggacgagagcauccacuacagcgagcuggugcaguucggcgccggcaagcgaccucaagcgaaggaggacguggacuacgugacccugaagcac

SEQ ID NO: 166 - RNA, 801 nt, Artificial Sequence
  Synthetic BCD17 nt. seq.
augcugggcggccugggcgugcugagaacccugccucugcuacuucucuuccugagcgaggccugccugggcccuggcugccaggcccugaugcuggagagggacccuccuagccugaccgugaaccugggcgaggaggccgugcugaccugcaagaacgacggcaagaacccuaacaucaccuggugguucagccugcagagcaacagcaccuggccuccuaugccucuagguccuggauugggcccaaugggcaagcuuaucuucccugaggugaacaagagccacagaggccuguacuggugccaggugaucgagagcaaggaggugaagagaagcugcggcaccuaccugagagugagaaagcaggugccuagaccuuuccuggacaugggagaaggcaccaagaacagaaucaucaccgccgagggcaucauccuguuauucugcgccguggugccuggcacccugcuucuguucagaaagcgguggcagaacgagaaguucggcguguacaaccuggccauggacgacaccgugagcuacgccguccugcgauucccugaaagcgacacacacggcgcuggcggugccagaagcccugccacccagggcccuccuccuaacgacgacgacacuguuaccuacagcgugcugcagaagagaaauaugggugacuacgagaacgugagcccuaacugcccugaggacgagagcauccacuacagcgagcuggugcaguucggugcuggaaagcggccucaggccaaggaggacguggacuacgugacccugaagcac

SEQ ID NO: 167 - RNA, 837 nt, Artificial Sequence
  Synthetic BCD18 nt. seq.
auggccacccuggugcugagcccugugccuugccacuggcugauguuccugcugcuccuuuugagcggcgagccugugccugccaugaccaagagcgaccagccuccuaucuuccagggcagcccuugcagcaagaucuggcagcacccuagauucgccgccaagaagagaagcagcauggugaaguuccacugccacaccgacuacagcggcgugaugaccugguucagacagaagggcaaccaacgaccucaggagcuguucccugaggacggccacaucagccagaccagaaacggcagcguguacacccugacccugcagaacauccaguacgaggacaacggcaucuacuucugccagcagaagugcaacagcaccgagccugacgugaccgacggcugcggcaccgagcugcuggugcugggcuucagcacccuggaccagcugaagagaagaaacacccugaaggacggcaucaucaugauccagacccugcugaucauccuguucaucaucgugccuaucuuccugcugcuggacaaggacuacaaccuggccauggacgacaccgugagcuacgccgugcugagauucccugagagcgacacgcacggcgccggaggcgccagaagcccugccacccagggcccuccuccuaacgacgacgauacaguuaccuacagcgugcugcagaagagaaacaugggcgacuacgagaacgugagcccuaacugcccugaggacgagagcauccacuacagcgagcuggugcaguucggcgccggcaagcgaccgcaggccaaggaggacguggacuacgugacccugaagcac

SEQ ID NO: 168 - protein, 322 aa, Artificial Sequence
  Synthetic BCD1 a.a. seq.
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLAEGLNLDDCSMAEDISRGLQGTYQDVGSLNIGDVQLEKPYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 169 - protein, 272 aa, Artificial Sequence
  Synthetic BCD2 a.a. seq.
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 170 - protein, 325 aa, Artificial Sequence
  Synthetic BCD3 a.a. seq.
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTAEGLDIDQTATAEDIVTLRTGEVKWSVGEHPGQEYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 171 - protein, 280 aa, Artificial Sequence
  Synthetic BCD4 a.a. seq.
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 172 - protein, 419 aa, Artificial Sequence
  Synthetic BCD5 a.a. seq.
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLGGGGSGGGGSYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 173 - protein, 600 aa, Artificial Sequence
  Synthetic BCD6 a.a. seq.
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLGGGGSGGGGSGGGGSFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 174 - protein, 652 aa, Artificial Sequence
  Synthetic BCD7 a.a. seq.
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQAGNVLSLPTPTSGLGRAQRWAAGLGGTAPSAGNPSSDVQADGALGSRSPPGVGPEEEEGEGAEEPDSEEDSEFYENDSNLGQDQLSQDGSGAENPEDEPLGPEDEDSFSNAESYENEDEELTQPVARTMDFLSPHGSAWDPSREATSLGSQSAEDMRGILYAAPQLRSIRGQPGPNHEEDADSYENMDNPDGPDPAWGGGGRMGTWSTRYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 175 - protein, 383 aa, Artificial Sequence
  Synthetic BCD8 a.a. seq.
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIVVALIYCRKKRISALPGYPECREMGETLPEKPANPTNPDEADKVGAENTITYSLLMHPDALEEPDDQNRI

SEQ ID NO: 176 - protein, 420 aa, Artificial Sequence
  Synthetic BCD9 a.a. seq.
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVVSLVYLKKKQVPALPGNPDHREMGETLPEEVGEYRQPSGGSVPVSPGPPSGLEPTSSSPYNPPDLEEAAKTEAENTITYSLLKHPEALDEETEHDYQNHI

SEQ ID NO: 177 - protein, 316 aa, Artificial Sequence
  Synthetic BCD10 a.a. seq.
MPGGLEALRALPLLLFLSYACLGPGCQALRVEGGPPSLTVNLGEEARLTCENNGRNPNITWWFSLQSNITWPPVPLGPGQGTTGQLFFPEVNKNHRGLYWCQVIENNILKRSCGTYLRVRNPVPRPFLDMGEGTKNRIITAEGIILLFCAVVPGTLLLFRKRWQNEKFGVDMPDDYEDENLAEGLNLDDCSMAEDISRGLQGTYQDVGNLHIGDAQLEKPYNPAMDDTVSYAILRFPESDTHNTGDAGTPATQAPPPNNSDSVTYSVIQKRPMGDYENVNPSCPEDESIHYSELVQFGAGKRPQAKEDVDYVTLKH

SEQ ID NO: 178 - protein, 324 aa, Artificial Sequence
  Synthetic BCD11 a.a. seq.
MATLVLSSMPCHWLLFLLLLFSGEPVPAMTSSDLPLNFQGSPCSQIWQHPRFAAKKRSSMVKFHCYTNHSGALTWFRKRGSQQPQELVSEEGRIVQTQNGSVYTLTIQNIQYEDNGIYFCKQKCDSANHNVTDSCGTELLVLGFSTLDQLKRRNTLKDGIILIQTLLIILFIIVPIFLLLDKDDGKAGMEEDHTAEGLNIDQTATAEDIVTLRTGEVKWSVGEHPGQEYNPAMDDTVSYAILRFPESDTHNTGDAGTPATQAPPPNNSDSVTYSVIQKRPMGDYENVNPSCPEDESIHYSELVQFGAGKRPQAKEDVDYVTLKH

SEQ ID NO: 179 - protein, 417 aa, Artificial Sequence
  Synthetic BCD12 a.a. seq.
MPPPRLLFFLLFLTPMEVRPQKSLLVEVEEGGNVVLPCLPDSSPVSSEKLAWYRGNQSTPFLELSPGSPGLGLHVGSLGILLVIVNVSDHMGGFYLCQKRPPFKDIWQPAWTVNVEDSGEMFRWNASDVRDLDCDLRNRSSGSHRSTSGSQLYVWAKDHPKVWGTKPVCAPRGSSLNQSLINQDLTVAPGSTLWLSCGVPPVPVAKASISWTHVHPRRPNVSLLSLSLGGEHPVREMWVWGSLLLLPQATALDEGTYYCLRGNLTIERHVKVIARSAVWLWLLRTGGWIVPVVTLVYVIFCMVSLVAFLYCGGGGSGGGGSYNPAMDDTVSYAILRFPESDTHNTGDAGTPATQAPPPNNSDSVTYSVIQKRPMGDYENVNPSCPEDESIHYSELVQFGAGKRPQAKEDVDYVTLKH

SEQ ID NO: 180 - protein, 643 aa, Artificial Sequence
  Synthetic BCD13 a.a. seq.
MPSPLPVSFLLFLTLVGGRPQKSLLVEVEEGGNVVLPCLPDSSPVSSEKLAWYRGNQSTPFLELSPGSPGLGLHVGSLGILLVIVNVSDHMGGFYLCQKRPPFKDIWQPAWTVNVEDSGEMFRWNASDVRDLDCDLRNRSSGSHRSTSGSQLYVWAKDHPKVWGTKPVCAPRGSSLNQSLINQDLTVAPGSTLWLSCGVPPVPVAKASISWTHVHPRRPNVSLLSLSLGGEHPVREMWVWGSLLLLPQATALDEGTYYCLRGNLTIERHVKVIARSAVWLWLLRTGGWIVPVVTLVYVIFCMVSLVAFLYCQRAFILRRKRKRMTDPARRFFKVTPPSGNGTQNQYGNVLSLPTSTSGQAHAQRWAAGLGSVPGSAGNPRIQVQDTGAQSHETGLEEEGEAAEEPDSEEGSEFYENDSNLGQDQVSQDGSGAENPEDEPMGPEEEDSFSNAESYENADEELAQPVGRMMDFLSPHGSAWDPSREASSLGSQSAEDMRGILYAAPQLHSIQSGPSHEEDADSYENMDKSDDLEPAWEGEGHMGTWGTTYNPAMDDTVSYAILRFPESDTHNTGDAGTPATQAPPPNNSDSVTYSVIQKRPMGDYENVNPSCPEDESIHYSELVQFGAGKRPQAKEDVDYVTLKH

SEQ ID NO: 181 - protein, 266 aa, Artificial Sequence
  Synthetic BCD14 a.a. seq.
MPGGLEALRALPLLLFLSYACLGPGCQALRVEGGPPSLTVNLGEEARLTCENNGRNPNITWWFSLQSNITWPPVPLGPGQGTTGQLFFPEVNKNHRGLYWCQVIENNILKRSCGTYLRVRNPVPRPFLDMGEGTKNRIITAEGIILLFCAVVPGTLLLFRKRWQNEKFGVYNPAMDDTVSYAILRFPESDTHNTGDAGTPATQAPPPNNSDSVTYSVIQKRPMGDYENVNPSCPEDESIHYSELVQFGAGKRPQAKEDVDYVTLKH

SEQ ID NO: 182 - protein, 279 aa, Artificial Sequence
  Synthetic BCD15 a.a. seq.
MATLVLSSMPCHWLLFLLLLFSGEPVPAMTSSDLPLNFQGSPCSQIWQHPRFAAKKRSSMVKFHCYTNHSGALTWFRKRGSQQPQELVSEEGRIVQTQNGSVYTLTIQNIQYEDNGIYFCKQKCDSANHNVTDSCGTELLVLGFSTLDQLKRRNTLKDGIILIQTLLIILFIIVPIFLLLDKDYNPAMDDTVSYAILRFPESDTHNTGDAGTPATQAPPPNNSDSVTYSVIQKRPMGDYENVNPSCPEDESIHYSELVQFGAGKRPQAKEDVDYVTLKH

SEQ ID NO: 183 - protein, 417 aa, Artificial Sequence
  Synthetic BCD16 a.a. seq.
MPSPLPVSLLLFLTLVGGRPQNSLLVEVEEGDNVVLSCLRDSSPVSSEKLAWYRGNQSTPFLELSLRSPDLGLHIGPLGILLVIVNVSDHRGGFYLCQKRPSFKDTWQPAWTVNVEDSGELFRWNASDLGDLDCDLGNRSSGSHRSTSGSQLYVWATDHPEVWKTKPVCAPREISLNQSLINQDLTVAPGSTLWLSCGVPPVPVTKGSISWTHVHPKTLNVSLLSLSLGGEHPVREMWVWGSLLLLPQAKASDEGTYYCLQGGLTIKMHVKVIARSAVWLWLLRTGGWIVPVVTLVYVIFCMVSMAAFLYFGGGGSGGGGSYNLAMDDTVSYAVLRFPESDTHGAGGARSPATQGPPPNDDDTVTYSVLQKRNMGDYENVSPNCPEDESIHYSELVQFGAGKRPQAKEDVDYVTLKH

SEQ ID NO: 184 - protein, 267 aa, Artificial Sequence
  Synthetic BCD17 a.a. seq.
MLGGLGVLRTLPLLLLFLSEACLGPGCQALMLERDPPSLTVNLGEEAVLTCKNDGKNPNITWWFSLQSNSTWPPMPLGPGLGPMGKLIFPEVNKSHRGLYWCQVIESKEVKRSCGTYLRVRKQVPRPFLDMGEGTKNRIITAEGIILLFCAVVPGTLLLFRKRWQNEKFGVYNLAMDDTVSYAVLRFPESDTHGAGGARSPATQGPPPNDDDTVTYSVLQKRNMGDYENVSPNCPEDESIHYSELVQFGAGKRPQAKEDVDYVTLKH

SEQ ID NO: 185 - protein, 279 aa, Artificial Sequence
  Synthetic BCD18 a.a. seq.
MATLVLSPVPCHWLMFLLLLLSGEPVPAMTKSDQPPIFQGSPCSKIWQHPRFAAKKRSSMVKFHCHTDYSGVMTWFRQKGNQRPQELFPEDGHISQTRNGSVYTLTLQNIQYEDNGIYFCQQKCNSTEPDVTDGCGTELLVLGFSTLDQLKRRNTLKDGIIMIQTLLIILFIIVPIFLLLDKDYNLAMDDTVSYAVLRFPESDTHGAGGARSPATQGPPPNDDDTVTYSVLQKRNMGDYENVSPNCPEDESIHYSELVQFGAGKRPQAKEDVDYVTLKH

SEQ ID NO: 186 - RNA, 57 nt, Artificial Sequence
  Synthetic 5′ UTR
gggaaauaagagagaaaagaagaguaagaagaaauauaagaccccggcgccgccacc

SEQ ID NO: 187 - RNA, 119 nt, Artificial Sequence
  Synthetic 3′ UTR
ugauaauaggcuggagccucgguggccuagcuucuugccccuugggccuccccccagccccuccuccccuuccugcacccguacccccguggucuuugaauaaagucugagugggcggc

SEQ ID NO: 188 - protein, 16 aa, Artificial Sequence
  Synthetic linker
  misc_feature (1)..(16): This region contains n repeats of “GGGS” wherein n = 1 - 4; some positions may or may not be present
GGGSGGGSGGGSGGGS

SEQ ID NO: 189 - RNA, 41 nt, Artificial Sequence
  Synthetic 5′ UTR
gggaaauaagagagaaaagaagaguaagaagaaauauaaga

SEQ ID NO: 190 - RNA, 47 nt, Artificial Sequence
  Synthetic 5′ UTR
gggaaauaagagagaaaagaagaguaagaagaaauauaagagccacc

SEQ ID NO: 191 - RNA, 54 nt, Artificial Sequence
  Synthetic 5′ UTR
gggaaauaagagagaaaagaagaguaagaagaaauauaagaccccggcgccacc

SEQ ID NO: 192 - DNA, 6 bp, Artificial Sequence
  Synthetic K0 Traditional Kozak sequence
gccrcc

SEQ ID NO: 193 - DNA, 6 bp, Artificial Sequence
  Synthetic EK
gccgcc

SEQ ID NO: 194 - DNA, 10 bp, Artificial Sequence
  Synthetic V1
ccccggcgcc

SEQ ID NO: 195 - DNA, 7 bp, Artificial Sequence
  Synthetic V2
ccccggc

SEQ ID NO: 196 - RNA, 9 nt, Artificial Sequence
  Synthetic Kozak consensus sequence R is a purine (adenine or guanine)
ccrccaugg

SEQ ID NO: 197 - RNA, 47 nt, Artificial Sequence
  Synthetic 5′ UTR
gggagaucagagagaaaagaagaguaagaagaaauauaagagccacc

SEQ ID NO: 198 - RNA, 42 nt, Artificial Sequence
  Synthetic 5′ UTR
gggagacaagcuuggcauuccgguacuguugguaaagccacc

SEQ ID NO: 199 - RNA, 47 nt, Artificial Sequence; identical to SEQ ID NO: 197
  Synthetic 5′ UTR

SEQ ID NO: 200 - RNA, 42 nt, Artificial Sequence; identical to SEQ ID NO: 198
  Synthetic 5′ UTR

SEQ ID NO: 201 - RNA, 47 nt, Artificial Sequence
  Synthetic 5′ UTR
gggaauuaacagagaaaagaagaguaagaagaaauauaagagccacc

SEQ ID NO: 202 - RNA, 47 nt, Artificial Sequence
  Synthetic 5′ UTR
gggaaauuagacagaaaagaagaguaagaagaaauauaagagccacc

SEQ ID NO: 203 - RNA, 47 nt, Artificial Sequence
  Synthetic 5′ UTR
gggaaauaagagaguaaagaacaguaagaagaaauauaagagccacc

SEQ ID NO: 204 - RNA, 47 nt, Artificial Sequence
  Synthetic 5′ UTR
gggaaaaaagagagaaaagaagacuaagaagaaauauaagagccacc

SEQ ID NO: 205 - RNA, 47 nt, Artificial Sequence
  Synthetic 5′ UTR
gggaaauaagagagaaaagaagaguaagaagauauauaagagccacc

SEQ ID NO: 206 - RNA, 47 nt, Artificial Sequence
  Synthetic 5′ UTR
gggaaauaagagacaaaacaagaguaagaagaaauauaagagccacc

SEQ ID NO: 207 - RNA, 47 nt, Artificial Sequence
  Synthetic 5′ UTR
gggaaauuagagaguaaagaacaguaaguagaauuaaaagagccacc

SEQ ID NO: 208 - RNA, 47 nt, Artificial Sequence
  Synthetic 5′ UTR
gggaaauaagagagaauagaagaguaagaagaaauauaagagccacc

SEQ ID NO: 209 - RNA, 47 nt, Artificial Sequence
  Synthetic 5′ UTR
gggaaauaagagagaaaagaagaguaagaagaaaauuaagagccacc

SEQ ID NO: 210 - RNA, 47 nt, Artificial Sequence
  Synthetic 5′ UTR
gggaaauaagagagaaaagaagaguaagaagaaauuuaagagccacc

SEQ ID NO: 211 - RNA, 92 nt, Artificial Sequence
  Synthetic 5′ UTR
ucaagcuuuuggacccucguacagaagcuaauacgacucacuauagggaaauaagagagaaaagaagaguaagaagaaauauaagagccacc

SEQ ID NO: 212 - RNA, 142 nt, Artificial Sequence
  Synthetic 3′ UTR
ugauaauaguccauaaaguaggaaacacuacagcuggagccucgguggccaugcuucuugccccuugggccuccccccagccccuccuccccuuccugcacccguacccccguggucuuugaauaaagucugagugggcggc

SEQ ID NO: 213 - RNA, 142 nt, Artificial Sequence
  Synthetic 3′ UTR
ugauaauaggcuggagccucgguggcuccauaaaguaggaaacacuacacaugcuucuugccccuugggccuccccccagccccuccuccccuuccugcacccguacccccguggucuuugaauaaagucugagugggcggc

SEQ ID NO: 214 - RNA, 142 nt, Artificial Sequence
  Synthetic 3′ UTR
ugauaauaggcuggagccucgguggccaugcuucuugccccuuccauaaaguaggaaacacuacaugggccuccccccagccccuccuccccuuccugcacccguacccccguggucuuugaauaaagucugagugggcggc

SEQ ID NO: 215 - RNA, 142 nt, Artificial Sequence
  Synthetic 3′ UTR
ugauaauaggcuggagccucgguggccaugcuucuugccccuugggccuccccccaguccauaaaguaggaaacacuacaccccuccuccccuuccugcacccguacccccguggucuuugaauaaagucugagugggcggc

SEQ ID NO: 216 - RNA, 142 nt, Artificial Sequence
  Synthetic 3′ UTR
ugauaauaggcuggagccucgguggccaugcuucuugccccuugggccuccccccagccccuccuccccuucuccauaaaguaggaaacacuacacugcacccguacccccguggucuuugaauaaagucugagugggcggc

SEQ ID NO: 217 - RNA, 142 nt, Artificial Sequence
  Synthetic 3′ UTR
ugauaauaggcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 218 - RNA, 142 nt, Artificial Sequence
  Synthetic 3′ UTR
ugauaauaggcuggagccucgguggccaugcuucuugccccuugggccuccccccagccccuccuccccuuccugcacccguacccccguggucuuugaauaaaguuccauaaaguaggaaacacuacacugagugggcggc

SEQ ID NO: 219 - RNA, 164 nt, Artificial Sequence
  Synthetic 3′ UTR
ugauaauaguccauaaaguaggaaacacuacagcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 220 - RNA, 164 nt, Artificial Sequence
  Synthetic 3′ UTR
ugauaauaguccauaaaguaggaaacacuacagcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 221 - RNA, 142 nt, Artificial Sequence
  Synthetic 3′ UTR
ugauaauaggcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 222 - DNA, 29 bp, Artificial Sequence
  Synthetic T7 promoter
  misc_feature (21)..(29): n is a, c, g, or t
taatacgactcactatagggnnnnnnnnn

SEQ ID NO: 223 - DNA, 18 bp, Artificial Sequence
  Synthetic T7 promoter
taatacgactcactatag

SEQ ID NO: 224 - DNA, 29 bp, Artificial Sequence
  Synthetic T7 promoter
  misc_feature (20)..(29): n is a, c, g, or t
taatacgactcactataagnnnnnnnnnn

SEQ ID NO: 225 - DNA, 29 bp, Artificial Sequence
  Synthetic T7 promoter
  misc_feature (20)..(29): n is a, c, g, or t
attatgctgagtgatattcnnnnnnnnnn

SEQ ID NO: 226 - protein, 22 aa, Artificial Sequence
  Synthetic 2A peptide
GSGATNFSLLKQAGDVEENPGP

SEQ ID NO: 227 - DNA, 66 bp, Artificial Sequence
  Synthetic nucleic acid encoding 2A peptide
ggaagcggagctactaacttcagcctgctgaagcaggctggagacgtggaggagaaccctggacct

SEQ ID NO: 228 - DNA, 108 bp, Artificial Sequence
  Synthetic nucleic acid encoding 2A peptide
tccggactcagatccggggatctcaaaattgtcgctcctgtcaaacaaactcttaactttgatttactcaaactggctggggatgtagaaagcaatccaggtccactc

SEQ ID NO: 229 - protein, 370 aa, Artificial Sequence
  Synthetic hSyk(nSH2-IA-cSH2-IB)
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLL

SEQ ID NO: 230 - protein, 274 aa, Artificial Sequence
  Synthetic hSyk(nSH2-IA-cSH2-GS)
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCGGGGSGGGGSGGGGS

SEQ ID NO: 231 - protein, 234 aa, Artificial Sequence
  Synthetic hSyk(nSH2-GS-cSH2-GS)
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFGGGGSGGGGSGGGGSGGGGSWFHGKISREESEQIVLIGSKTNGKFLIRARDNNGSYALCLLHEGKVLHYRIDKDKTGKLSIPEGKKFDTLWQLVEHYSYKADGLLRVLTVPCGGGGSGGGGSGGGGS

SEQ ID NO: 232 - DNA, 1926 bp, Artificial Sequence
  Synthetic BCD19 n.t. seq.
atggccagcagcggcatggccgacagcgccaaccacctgcctttcttcttcggcaacatcaccagagaggaggccgaggactacctggtgcagggcggcatgagcgacggcctgtacctgctgagacagagcagaaactacctgggcggcttcgccctgagcgtggcccacggcagaaaggcccaccactacaccatcgagagagagctgaacggcacctacgccatcgccggcggcagaacccacgccagccctgccgacctgtgccactaccacagccaggagtcagacggcctggtgtgcctgctgaagaagcctttcaacagacctcagggcgtgcagcctaagaccggccctttcgaggacctgaaggagaacctgatcagagagtacgtgaagcagacctggaacctgcagggccaggccctggagcaggccatcatcagccagaagcctcagctggagaagctgatcgccaccaccgcccacgagaagatgccttggttccacggcaagatctcaagggaggagtcggagcagatcgtgctgatcggcagcaagaccaacggcaagttcctcattagagccagagacaacaacggcagctacgccctgtgtctcctgcacgagggcaaggtgctgcactacagaatcgacaaggacaagaccggcaagctgagcatccctgagggcaagaagttcgacaccctgtggcagctggtggagcactacagctacaaggcagacggactgctgagagtgctgaccgtgccttgccagaagatcggcacccagggcaacgtgaacttcggcggtagaccacagctgcctggcagccaccctgccacctggtctgccggtggcattatcagcagaatcaagagctacagcttccctaagcctggccacagaaagagttccccagcacaaggtaacagacaggagagcaccgtgagcttcaacccttacgagcctgagctggccccttgggccgccgacaagggccctcagagagaggccctgcctatggacaccgaggtgtacgagagcccttacgccgaccctgaggagatcagacctaaggaggtgtacctggacagaaagctgctgttctgggaggagttcgagagcctgcagaagcaggaggtgaagaacctgcaccagagactggagggccaaaggcctgagaacaagggaaagaacagatacaagaacatattacctttcgaccacagcagagtgattctgcagggcagagacagcaacatccctggcagcgactacatcaacgccaactacattaagaaccagctgctgggccctgacgagaacgccaagacctacatcgccagccagggctgcctggaggccaccgtgaacgacttctggcagatggcgtggcaggagaactcaagagtgatcgtgatgaccacccgggaggtggagaagggcagaaacaagtgcgtgccttactggcctgaggtgggcatgcagagagcctacggcccttacagcgtgaccaactgcggcgagcacgacaccaccgagtacaagctgagaaccctgcaggtgagccctctggacaacggcgatctgatcagggagatctggcactaccagtatttgtcctggcctgaccacggcgtgcctagcgagcctggcggcgtgctgagcttcctggaccagatcaaccagcgtcaagaaagtctccctcacgccggccctatcatcgtgcactgcagcgccggcatcggaaggaccggcaccatcatcgtgatcgacatgctgatggagaacatcagcaccaagggcctggactgcgacatcgacatccagaagaccatccagatggtgagagcccagagatccggcatggtgcagaccgaggcccagtacaagttcatctacgtggccatcgcccagttc

SEQ ID NO: 233 - DNA, 1638 bp, Artificial Sequence
  Synthetic BCD20 n.t. seq.
atggccagcagcggcatggccgacagcgccaaccacctgcctttcttcttcggcaacatcaccagagaggaggccgaggactacctggtgcagggcggcatgagcgacggcctgtacctgctgagacagagcagaaactacctgggcggcttcgccctgagcgtggcccacggcagaaaggcccaccactacaccatcgagagagagctgaacggcacctacgccatcgccggcggcagaacccacgccagccctgccgacctgtgccactaccacagccaggagtcagacggcctggtgtgcctgctgaagaagcctttcaacagacctcagggcgtgcagcctaagaccggccctttcgaggacctgaaggagaacctgatcagagagtacgtgaagcagacctggaacctgcagggccaggccctggagcaggccatcatcagccagaagcctcagctggagaagctgatcgccaccaccgcccacgagaagatgccttggttccacggcaagatcagcagagaggagagcgagcagatcgtgctgatcggcagcaagaccaacggcaagttcctcatcagagccagagacaacaacggcagctacgccctgtgtttgctgcacgagggcaaggtgctgcactacagaatcgacaaggacaagaccggcaagctgagcatccctgagggcaagaagttcgacaccctgtggcagctggtggagcactacagctacaaggccgacggcctgctgagagtgctgaccgtgccttgcggaggcggaggcagcggaggtggaggttcaggaggcggcggaagcttctgggaggagttcgagagcctgcagaagcaggaggtgaagaacctgcaccagagactggagggccagcggcctgagaacaagggcaagaacagatacaagaacatcctgcctttcgaccacagcagagtgatcctgcagggcagagacagcaacatccctggcagcgactacatcaacgccaactacatcaagaaccagctgctgggccctgacgagaacgccaagacctacatcgccagccagggctgcctggaggccaccgtgaacgacttctggcaaatggcttggcaggagaattctcgcgtgatcgtgatgacgacccgagaggtggagaagggcagaaacaagtgcgtgccttactggcctgaggtgggcatgcagagagcctacggcccttacagcgtgaccaactgcggcgagcacgacaccaccgagtacaagctgagaaccctgcaggtgagccctctggacaacggcgatcttattcgggagatctggcactaccagtacctgagctggcctgaccacggcgtgcctagcgagcctggcggcgtgctgagcttcctggaccagatcaaccagagacaggagagcctgcctcacgccggccctatcatcgtgcactgcagcgccggcatcggcagaaccggcaccatcatcgtgatcgacatgctgatggagaacatcagcaccaagggcctggactgcgacatcgacatccagaagaccatccagatggtgagagcccagagaagcggcatggtgcagaccgaggcccagtacaagttcatctacgtggccatcgcccagttc

SEQ ID NO: 234 - DNA, 1518 bp, Artificial Sequence
  Synthetic BCD21 n.t. seq.
atggccagcagcggcatggccgacagcgccaaccacctgcctttcttcttcggcaacatcaccagagaggaggccgaggactacctggtgcagggcggcatgagcgacggcctgtacctgctgagacagagcagaaactacctgggcggcttcgccctgagcgtggcccacggcagaaaggcccaccactacaccatcgagagagagctgaacggcacctacgccatcgccggcggcagaacccacgccagccctgccgacctgtgccactaccacagccaggagagcgacggtctggtgtgcctgctgaagaagcctttcggaggcggaggcagcggaggaggaggctccggtggcggtggcagtggcggcggcggaagctggttccacggcaagatctctagggaagaatctgagcagatcgtgctgatcggcagcaagaccaacggcaagttcctgatcagagccagagacaacaacggcagctacgccctgtgcttactgcacgagggcaaggtgctgcactacagaatcgacaaggacaagaccggcaagctgagcatccctgagggcaagaagttcgacaccctgtggcagctggtggagcactacagctacaaggccgacggactgttgagagtgctgaccgtgccttgcggaggcggcggatctggaggtggtggctcaggtggcggaggctctttctgggaggagttcgagagcctgcagaagcaggaggtgaagaacctgcaccagagactggagggccagcggcctgagaacaagggaaagaacagatacaagaacatcctgccattcgaccacagcagagtgatcctgcagggcagagacagcaacatccctggcagcgactacatcaacgccaattacataaagaaccagctgctgggccctgacgagaacgccaagacctacatcgccagccagggctgcctggaggccaccgtgaacgacttctggcagatggcctggcaggagaatagccgtgtgatcgtgatgacgacacgggaggtggagaagggcagaaacaagtgcgtgccttactggcctgaggtgggcatgcagagagcctacggcccttacagcgtgaccaactgcggcgagcacgacaccaccgagtacaagctgagaaccctgcaggtgagccctctggacaacggcgacctcatccgcgagatctggcactaccagtacctgagctggcctgaccacggcgtgcctagcgagcctggcggcgtgctgagcttcctggaccagatcaaccagagacaggaatccctccctcacgccggccctatcatcgtgcactgcagcgccggcatcggaaggaccggcaccatcatcgtgatcgacatgctgatggagaacatcagcaccaagggcctggactgcgacatcgacatccagaagaccatccagatggtgagagcccagagatctggaatggtgcagaccgaggcccagtacaagttcatctacgtggccatcgcccagttc

SEQ ID NO: 235 - DNA, 2319 bp, Artificial Sequence
  Synthetic BCD22 n.t. seq.
atgcctcctcctagactgctgttcttccttctgttcctgacccctatggaggtgagacctgaggagcctctggtggtgaaggtggaggagggcgacaacgccgtgctgcagtgcctgaagggcaccagcgacggccctacccagcagctgacctggagcagagagagccctctgaagcctttcctgaagctgagcctgggcctgcctggcctgggcatccacatgcgtcctctggccatctggctgttcatcttcaacgtgagccagcagatgggcggcttctacctgtgccagcctggccctcctagcgagaaggcctggcagccaggttggaccgtgaacgtggagggcagcggcgagctgttccggtggaacgtgagcgacctgggcggcctgggttgcggcctgaagaacagaagcagcgagggccctagcagccctagcggcaagctgatgagccctaagctgtacgtgtgggccaaggacagacctgagatctgggagggagagcctccttgcctgcctccacgcgacagcctgaaccagagcctgagccaggacctgaccatggcccctggctctaccctgtggctgagctgcggcgtgcctcctgacagcgtgagcagaggccctttgagctggacccacgtgcaccctaagggaccaaagagccttctgtcgctggagctgaaggacgatcgtccagccagagacatgtgggtgatggagacaggcctgctgctgcctagagccaccgcccaggacgccggcaagtactactgccacagaggcaacctcaccatgagcttccacctggagatcaccgccagacctgtgctgtggcactggctgctgagaaccggcggctggaaggtgagcgccgtgaccctggcctacctgatcttctgcctgtgtagcctcgtgggaatactgcaccttggcggaggtggtagtggtggcggcggctctatgagcgccatccaggccgcttggccaagtggtaccgagtgcatcgccaagtacaacttccacggcaccgccgagcaggatctacctttctgcaagggcgacgtgctgaccatcgtggccgtaaccaaggaccctaacgcctacaaggccaagaacaaggtgggcagagagggcatcatccctgccaactacgtgcagaagcgggagggtgtgaaggccggcaccaagctgtcactgatgccttggttccacggaaagatcaccagagagcaggccgagaggctattgtatccgcctgaaactggccttttccttgtcaaggagagcaccaactaccctggcgactacacgctctgcgtttcctgtgacggcaaggtggaacactacagaatcatgtaccacgcctccaagctatctatcgacgaggaggtgtacttcgagaacctgatgcagctggtggcccactacacgagcgacgccgacggcctgtgcaccagactgatcaagcctaaggtcatggaaggcaccgttgccgctcaggacgagttctaccggtccggctgggccctgaacatgaaggagttaaagctcctgcagaccatcggaaagggagagttcggcgacgtcatgctgggtgattacagaggaaataaggttgctgtgaagtgcatcaagaacgacgcaaccgcccaagccttcctggccgaggccagcgtgatgacccagctgagacacagcaacctggtgcagctcttgggagtgatcgtcgaggagaagggaggcctgtacatcgtgaccgagtacatggccaagggaagcttagtggactacctgcgttccaggggccgttccgttctcggaggtgactgcctcttaaagttcagcctggacgtgtgcgaggccatggagtacctggaaggaaacaacttcgtgcaccgtgacctggccgccagaaacgtgctggtgagcgaggacaacgtagctaaggtgtctgatttcggcctgactaaggaggccagttctactcaggacaccggtaagctcccggtaaagtggaccgcccctgaggccctgagagagaagaagttcagtaccaagagcgacgtgtggagcttcggcatcctgctgtgggagatctacagtttcggcagagtgccttaccctagaattccattaaaggacgtggtacctagggttgagaagggctacaagatggacgccccggacggctgccctcctgccgtgtacgaggtgatgaagaactgctggcacctggacgccgccatgcggccgagcttcctgcagctgagggaacaactggagcacatcaagacccacgagctccatctg

SEQ ID NO: 236 - DNA, 1896 bp, Artificial Sequence
  Synthetic BCD23 n.t. seq.
atgcctggcggccctggcgtgctgcaggccctgcctgccaccatcttcctgctgttcctcctgagcgccgtgtacctgggacctggctgccaggccctgtggatgcacaaggtcccagcaagcctgatggtgagcctgggcgaggacgcccacttccagtgccctcacaacagcagcaacaacgccaacgtgacctggtggagagtgctgcacggcaactacacctggcctccagaattcctcggcccaggcgaagatcctaacggcaccctgatcatccagaacgtgaacaagagccacggcggcatctacgtgtgcagagtgcaggagggcaacgagagctaccagcagagctgcggcacctacctgagagtgagacagcctcctcctagacctttcctggacatgggtgagggcaccaagaacagaatcatcaccgccgagggcatcattttactcttctgcgccgtggtgcctggtaccctattattgttcagaaagaggtggcagaacgagaagctgggtctgggcggtggaggcagcggcatgagcgccatccaggccgcctggcctagcggcaccgagtgcatcgccaagtacaacttccacggaactgccgagcaggacctgcctttctgcaagggcgacgtgctgaccatcgtggccgtgaccaaggacccaaacgcctacaaggctaagaataaggtgggcagagagggtattattcctgccaactacgtgcagaagagggaaggcgtgaaggccggcactaagctgagtcttatgccttggttccacggtaagatcaccagagagcaggccgagagactgctgtacccacctgaaaccggcttgttcctggtgaaggagagcaccaactatccaggcgactacaccctgtgcgtgagctgcgacggcaaggtggagcactatcgcatcatgtaccacgcctcaaagttgtccatcgacgaggaggtgtacttcgagaacctgatgcagctggtggcccactacaccagcgacgccgacggcctgtgcaccagactgatcaagcctaaggtgatggaaggcaccgtggccgcccaggacgagttctacagaagcggttgggcgctgaacatgaaggagctgaagctgctgcagaccatcggaaagggtgagttcggtgacgtgatgctgggagattatagaggcaacaaggtggccgtaaagtgcatcaagaacgacgccacagcccaggccttcctggccgaggccagcgtgatgacccagctgagacacagcaacctggtgcagttattgggcgtgatcgtggaggagaagggcggcctgtacatcgtgaccgagtacatggccaagggctctcttgtggactacctccgtagcagaggcagaagcgttcttggaggtgactgcctgctgaagttcagcctggacgtgtgcgaggccatggagtacctagaaggtaataacttcgtgcacagggacctggccgccagaaacgtgctggtgagcgaggacaacgtagccaaggttagcgacttcggcctgacgaaggaagcaagtagcacccaggacaccggcaagctgcctgtgaagtggaccgcccctgaggccctgagagagaagaagttctccacaaagagcgacgtgtggagcttcggcatactgctgtgggagatctactcattcggccgagttccttaccctagaatccctctgaaggacgtggtccctagagtcgagaagggatacaagatggacgctcctgacggctgccctcctgcagtctacgaggtgatgaagaactgctggcacctggacgccgccatgcgccctagcttcctgcaacttagggagcagctggagcacatcaagacccacgagctgcacctg

SEQ ID NO: 237 - DNA, 1917 bp, Artificial Sequence
  Synthetic BCD24 n.t. seq.
atggccagactggccctgtcacctgtgcctagccactggatggtggccctcctgctgctgctgtctgctgaaccagtgcctgccgccagaagcgaggacagatacagaaaccctaaagggagcgcctgcagcagaatctggcagagcccgagattcatcgccagaaagagaggcttcaccgtgaagatgcactgctacatgaatagcgcaagcggcaacgtgagctggctgtggaagcaggagatggacgagaacccgcagcagctgaagctggagaagggcagaatggaggagagccagaacgagagcctggctaccctgaccattcaaggaatcagattcgaggacaacggcatctacttctgccagcagaagtgcaacaacaccagcgaggtgtaccagggctgcggcaccgaactgagagtgatgggcttcagcaccctggcccaacttaagcagagaaacaccctgaaggacggcatcatcatgatccagaccctgctgatcatcctgttcatcatcgtgcctatcttcctgctgctggacaaggacggcggcggcggtagcatgagcgccatccaggccgcctggcctagcggcaccgagtgcatcgccaagtacaacttccacggcaccgccgagcaggacctgcctttctgcaagggcgacgtgctcaccatcgtcgccgttaccaaggaccctaacgcctacaaggccaagaacaaggtgggcagagagggaatcattcctgccaactacgtgcagaagagagagggcgtgaaggccggcaccaagctaagcctgatgccttggttccacggcaagatcacaagagagcaggccgagagactgctgtaccctcctgagactggcctgttcctggtgaaggaaagcaccaactaccctggcgactacaccctgtgcgtgagctgcgacggcaaggtggagcactaccgaatcatgtaccacgccagcaagctgagcatcgacgaggaggtgtacttcgagaacctgatgcagctggtggcccactacacctctgacgccgacggcctgtgcaccagactgatcaagccaaaggtgatggagggcaccgtggccgcccaggacgagttctacagatcaggctgggcccttaacatgaaggagttgaagctgctgcagaccatcggcaagggcgagttcggcgacgtgatgctgggcgactacagaggcaacaaggtggccgtgaagtgcatcaagaacgacgccaccgcccaagccttcctggccgaggccagcgtgatgacccagctgcgacacagcaatctggtgcagctgctgggcgtgatcgtggaggagaagggcggcctgtacatcgtgactgagtacatggccaagggcagcctggtggattatctgagatcaaggggcagaagcgtgctgggcggcgactgcctgctgaaattcagcctggacgtctgcgaagccatggagtacctggaggggaacaacttcgtgcaccgcgacctggccgccagaaacgtgctggtgtccgaggacaacgtggctaaagtgagcgacttcggcctgaccaaggaggccagcagcacccaggacaccggcaagctgcctgtgaagtggaccgcccctgaggccctgagagagaagaagttcagcaccaagagcgacgtgtggagcttcggcattctgctgtgggagatttacagctttggcagagtgccttaccctagaatccctctcaaagacgtggtgcctagagtggagaagggctacaagatggacgcccctgacggctgccctcctgccgtgtacgaggtgatgaagaactgctggcacctggacgccgccatgcggcctagcttcttacagctgagagagcagctggagcacatcaagacccacgaactgcacctg

SEQ ID NO: 238 - protein, 642 aa, Artificial Sequence
  Synthetic amino acid sequence
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 239 - protein, 546 aa, Artificial Sequence
  Synthetic BCD20 a.a. seq.
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCGGGGSGGGGSGGGGSFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 240 - protein, 506 aa, Artificial Sequence
  Synthetic BCD21 a.a. seq.
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFGGGGSGGGGSGGGGSGGGGSWFHGKISREESEQIVLIGSKTNGKFLIRARDNNGSYALCLLHEGKVLHYRIDKDKTGKLSIPEGKKFDTLWQLVEHYSYKADGLLRVLTVPCGGGGSGGGGSGGGGSFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 241 - protein, 772 aa, Artificial Sequence
  Synthetic BCD22 a.a. seq.
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLGGGGSGGGGSMSAIQAAWPSGTECIAKYNFHGTAEQDLPFCKGDVLTIVAVTKDPNAYKAKNKVGREGIIPANYVQKREGVKAGTKLSLMPWFHGKITREQAERLLYPPETGLFLVKESTNYPGDYTLCVSCDGKVEHYRIMYHASKLSIDEEVYFENLMQLVAHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELH

SEQ ID NO: 242 - protein, 631 aa, Artificial Sequence
  Synthetic BCD23 a.a. seq.
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLGGGGSGMSAIQAAWPSGTECIAKYNFHGTAEQDLPFCKGDVLTIVAVTKDPNAYKAKNKVGREGIIPANYVQKREGVKAGTKLSLMPWFHGKITREQAERLLYPPETGLFLVKESTNYPGDYTLCVSCDGKVEHYRIMYHASKLSIDEEVYFENLMQLVAHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELH

SEQ ID NO: 243 - protein, 638 aa, Artificial Sequence
  Synthetic BCD24 a.a. seq.
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDGGGGSMSAIQAAWPSGTECIAKYNFHGTAEQDLPFCKGDVLTIVAVTKDPNAYKAKNKVGREGIIPANYVQKREGVKAGTKLSLMPWFHGKITREQAERLLYPPETGLFLVKESTNYPGDYTLCVSCDGKVEHYRIMYHASKLSIDEEVYFENLMQLVAHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELH

SEQ ID NO: 244 - DNA, 30 bp, Artificial Sequence
  Synthetic nucleotide
  misc_feature (1)..(30): This region contains n repeats of “ccg” wherein n = 1 - 10; some positions may or may not be present
ccgccgccgccgccgccgccgccgccgccg

SEQ ID NO: 245 - DNA, 30 bp, Artificial Sequence
  Synthetic nucleotide
  misc_feature (1)..(30): This region contains n repeats of “gcc” wherein n = 1 - 10; some positions may or may not be present
gccgccgccgccgccgccgccgccgccgcc